(19) United States
(12) Patent Application Publication
(10) Pub. No.: US 2025/0346653 A1
(43) Pub. Date: Nov. 13, 2025

(54) BISPECIFIC ANTIBODIES AND METHOD OF TREATING OCULAR DISEASE

(71) Applicant: Broadwing Bio Inc., South San Francisco, CA (US)

(72) Inventor: Andrew Peterson, South San Francisco, CA (US)

(21) Appl. No.: 18/860,132

(22) PCT Filed: Apr. 28, 2023
(86) PCT No.: PCT/US23/20365

§ 371 Date: Oct. 25, 2024

Related U.S. Application Data
(60) Provisional application No. 63/336,766, filed on Apr. 29, 2022.

(51) Int. Cl. C07K 16/18

(52) U.S. Cl. CPC C07K 16/18; C07K 2317/31; C07K 2317/33; C07K 2317/565; C07K 2317/76; C07K 2317/92; C07K 2317/94

(57) ABSTRACT

The present disclosure relates to the treatment and/or prevention of age-related macular degeneration (AMD), including both neovascular AMD (“wet” AMD) and non-neovascular AMD (“dry” AMD), as well as advanced forms of non-neovascular AMD (geographic atrophy or GA). In particular, the present disclosure provides novel therapeutic bispecific antibodies that target components of the alternative pathway of the complement activation system.

### CROSS REFERENCE TO RELATED APPLICATIONS

This application claims priority to and the benefit of U.S. Provisional Patent Application No. 63/336,766 filed Apr. 29, 2022, which is incorporated herein by reference in its entirety for all purposes.

### SEQUENCE LISTING

The text of the computer readable sequence listing filed herewith, titled “40820_601_SequenceListing,” created on Apr. 27, 2023, having a file size of 1,823,343 bytes bytes, is hereby incorporated by reference in its entirety.

### FIELD

Embodiments of the present disclosure relate to the treatment and/or prevention of age-related macular degeneration (AMD), including both neovascular AMD (“wet” AMD) and non-neovascular AMD (“dry” AMD), as well as advanced forms of non-neovascular AMD (geographic atrophy or GA). In particular, the present disclosure provides novel therapeutic bispecific antibodies that target components of the alternative pathway of the complement activation system.

### BACKGROUND

Age-Related Macular Degeneration (AMD) is the leading cause of severe vision loss in adults over age 50. The Centers for Disease Control and Prevention estimate that 1.8 million people have AMD and another 7.3 million are at substantial risk for vision loss from AMD. This eye disease occurs when there are changes to the macula, a small portion of the retina that is located on the inside back layer of the eye. AMD is a loss of central vision that can occur in two forms: “dry” (atrophic or non-neovascular) and “wet” (exudative or neovascular). Most people with macular degeneration have the dry form. Geographic atrophy (GA) is considered the late stage of the dry form of AMD, characterized by focal and progressive loss of photoreceptors. GA is less common than neovascular AMD, and it is responsible for 10-20% of cases of legal blindness in this condition, affecting more than 5 million people worldwide. Currently there is no approved or effective treatment to prevent either the onset or progression of GA; however, in recent years, significant progress has been made in understanding the pathogenesis of GA, which has led to a number of new potential therapies. Treatments of wet AMD are rapidly evolving and some now target specific biochemical events such as angiogenesis. The use of pharmacotherapy targeting vascular endothelial growth factor (VEGF) has been shown to slow vision loss and even lead to vision improvement in some patients with wet macular degeneration. However, the ability to treat both wet and dry AMD with a single biologic has not been established.

### SUMMARY

Embodiments of the present disclosure include a bispecific antibody, or an antigen binding fragment thereof, which specifically binds human Complement Factor H Related 4 (CFHR) and human Complement Component 3 (C3). In some embodiments, the human CFHR4 is the CFHR4 b variant (CFHR4b), which is optionally a polypeptide comprising or consisting of the amino acid sequence of SEQ ID NO: 580. In some embodiments, the human C3 is the b variant of C3 (C3b), which is optionally a polypeptide comprising or consisting of the amino acid sequence of SEQ ID NO: 1645.

In some embodiments, the bispecific antibody or fragment thereof is monoclonal, optionally recombinant. In some embodiments, the bispecific antibody or fragment thereof is human, humanized, or chimeric. In some embodiments, the bispecific antibody or fragment thereof is a full length antibody, a single chain antibody, a single chain variable fragment (scFv), a variable fragment (Fv), a fragment antigen-binding region (Fab), a Fab-C, a Fab′-SH, a (Fab′) 2, a single-domain antibody (sdAb), a VHH antibody, a nanobody, a camelid-derived single-domain antibody, a shark IgNAR-derived single-domain antibody fragment (VNAR), a diabody, a triabody, an anticalin or an aptamer, optionally wherein the antibody is a full length antibody comprising an Fc region such as a human IgG1, IgG2, IgG3 or IgG4 region.

In some embodiments, the bispecific antibody or fragment thereof is conjugated to at least one additional moiety, optionally selected from: an antigen binding moiety, such as an antibody or antigen-binding fragment thereof, which is capable of specific binding to a target which is not human CFHR4 or human C3, preferably wherein said target is expressed in the human eye; a therapeutic or cytotoxic moiety; a detection moiety; a purification moiety; a half-life extension moiety, optionally a polypeptide that is at least 20 amino acids in length and comprises any combination of G, A, S T, E, and P residue, which polypeptide is conjugated to the C- or N-terminus of the antibody.

In some embodiments, the bispecific antibody or fragment thereof is a polypeptide comprising: one, two or all three HCDRs of any one of the exemplary CFHR4 antibodies the sequences of which are provided in Table 3; and one, two or all three of the corresponding LCDRs of said exemplary antibody; and/or a VH sequence having at least 90% identity to the VH sequence of any one of the exemplary CFHR4 antibodies the sequences of which are provided in Table 4; and optionally a VL sequence having at least 90% identity to the corresponding VL sequence of said exemplary antibody, preferably wherein variation is not permitted in the HCDRs or LCDRs; and/or all six CDRs of any one of the exemplary CFHR4 antibodies the sequences of which are provided in Table 3; the VHI and VL sequences of any one of the exemplary CFHR4 antibodies the sequences of which are provided in Table 4; and/or the full length heavy chain (VH+constant) sequence of any one of the exemplary CFHR4 antibodies the sequences of which are provided in Table 5; and optionally the corresponding full length light chain (VL+constant) sequence of said exemplary antibody.

In some embodiments, the bispecific antibody or fragment thereof is a polypeptide comprising: one, two or all three HCDRs of any one of the exemplary C3 antibodies the sequences of which are provided in Table 6; and one, two or all three of the corresponding LCDRs of said exemplary antibody; and/or a VH sequence having at least 90% identity to the VH sequence of any one of the exemplary C3 antibodies the sequences of which are provided in Table 7; and optionally a VL sequence having at least 90% identity to the corresponding VL sequence of said exemplary antibody provided in Table 8, preferably wherein variation is not permitted in the HCDRs or LCDRs; and/or all six CDRs of any one of the exemplary C3 antibodies the sequences of which are provided in Table 6; the VH and VL sequences of any one of the exemplary C3 antibodies the sequences of which are provided in Tables 7 and 8; and/or the full length heavy chain (VH+constant) sequence of any one of the exemplary C3 antibodies the sequences of which are provided in Table 9; and optionally the corresponding full length light chain (VL+constant) sequence of said exemplary antibody provided in Table 10.

Embodiments of the present disclosure also include a polynucleotide encoding the bispecific antibody or fragment thereof. In some embodiments, the polynucleotide comprises or consists of a nucleic acid sequence having at least 70%, 80%, 90%, or 100% identity to a nucleic acid sequence of any one of the exemplary antibodies the sequences of which are provided in Tables 5, 9, and 10. Embodiments of the present disclosure also include an expression vector comprising the polynucleotide, which is optionally an adeno-associated virus (AAV) vector, a lentiviral (LV) vector, a herpes simplex virus (HSV) vector, or a retrovirus vector.

Embodiments of the present disclosure also include a pharmaceutical composition comprising the bispecific antibody or fragment thereof, the corresponding polynucleotide, or the vector comprising the polynucleotide. In some embodiments, the composition comprises at least one pharmaceutically acceptable carrier, diluent or preservative; and/or at least one additional active ingredient.

In some embodiments, the pharmaceutical composition is suitable for ocular administration to a subject, optionally by delivery using a conjunctival insert, a contact lens, a gel, a nanoparticle, a mucoadhesive polymer, an ointment, a solution, a suspension, eye drops, and/or an implant, preferably by injection into the vitreous fluid.

Embodiments of the present disclosure also include the bispecific antibody or fragment thereof, the corresponding polynucleotide, the vector comprising the polynucleotide, or the pharmaceutical composition, for use as a medicament, optionally for use in a method of treating a disease of the eye in a subject. In some embodiments, the disease is characterized by increased activation of the complement system, in particular the alternative pathway, and in particular within the eye of the subject, for example in drusen or retinal pigment epithelium (RPE) cells of the subject. In some embodiments, the method comprises ocular administration of the antibody, preferably by injection into the vitreous fluid, and said administration preferably relieves at least one symptom in the subject selected from visual distortion, reduced central vision, blurred vision, and/or difficulty adapting to low light. In some embodiments, the disease is age-related macular degeneration (AMD), including dry AMD, which may be at an early, intermediate or advanced stage (the latter otherwise known as geographic atrophy, GA).

In accordance with the above, embodiments of the present disclosure include a bispecific antibody comprising a first antigen-binding site directed against Complement Factor H Related 4 (CFHR4) and a second antigen-binding site directed against Complement Component 3 (C3). In some embodiments, the first anti-CFHR4 antigen-binding site comprises a heavy chain variable region (VH) comprising complementarity determining regions (CDRs) HCDR1, HCDR2, and HCDR3 from at least one of SEQ ID NOs: 260-265, 284-293, 324-334, and 368-380; and a light chain variable region (VL) comprising complementarity determining regions (CDRs) LCDR1, LCDR2, and LCDR3 from at least one of SEQ ID NOs: 272-277, 304-313, 346-356, and 394-406; and the second anti-C3 antigen-binding site comprises a heavy chain variable region (VH) comprising complementarity determining regions (CDRs) HCDR1, HCDR2, and HCDR3; and a light chain variable region (VL) comprising complementarity determining regions (CDRs) LCDR1, LCDR2, and LCDR3.

Embodiments of the present disclosure include a bispecific antibody comprising a first antigen-binding site directed against Complement Factor H Related 4 (CFHR4) and a second antigen-binding site directed against Complement Component 3 (C3). In some embodiments, the first anti-CFHR4 antigen-binding site comprises a heavy chain variable region (VH) comprising complementarity determining regions (CDRs) HCDR1, HCDR2, and HCDR3; and a light chain variable region (VL) comprising complementarity determining regions (CDRs) LCDR1, LCDR2, and LCDR3; and the second anti-C3 antigen-binding site comprises a heavy chain variable region (VH) comprising complementarity determining regions (CDRs) HCDR1, HCDR2, and HCDR3 from at least one of SEQ ID NOs: 1127-1200; and a light chain variable region (VL) comprising complementarity determining regions (CDRs) LCDR1, LCDR2, and LCDR3 from at least one of SEQ ID NOs: 1275-1348.

Embodiments of the present disclosure include a bispecific antibody comprising a first antigen-binding site directed against Complement Factor H Related 4 (CFHR4) and a second antigen-binding site directed against Complement Component 3 (C3). In some embodiments, the first anti-CFHR4 antigen-binding site comprises a heavy chain variable region (VH) comprising complementarity determining regions (CDRs) HCDR1, HCDR2, and HCDR3 from at least one of SEQ ID NOs: 260-265, 284-293, 324-334, and 368-380; and a light chain variable region (VL) comprising complementarity determining regions (CDRs) LCDR1, LCDR2, and LCDR3 from at least one of SEQ ID NOs: 272-277, 304-313, 346-356, and 394-406; and the second anti-C3 antigen-binding site comprises a heavy chain variable region (VH) comprising complementarity determining regions (CDRs) HCDR1, HCDR2, and HCDR3 from at least one of SEQ ID NOs: 1127-1200; and a light chain variable region (VL) comprising complementarity determining regions (CDRs) LCDR1, LCDR2, and LCDR3 from at least one of SEQ ID NOs: 1275-1348.

In some embodiments, the anti-CFHR4 HCDR1 comprises the amino acid sequence of SEQ ID NO: 2; the anti-CFHR4 HCDR2 comprises the amino acid sequence of SEQ ID NO: 9; and the anti-CFHR4 HCDR3 comprises the amino acid sequence of SEQ ID NO: 15. In some embodiments, the anti-CFHR4 HCDR1 comprises the amino acid sequence of SEQ ID NO: 3; the anti-CFHR4 HCDR2 comprises the amino acid sequence of SEQ ID NO: 10; and the anti-CFHR4 HCDR3 comprises the amino acid sequence of SEQ ID NO: 16. In some embodiments, the anti-CFHR4 HCDR1 comprises the amino acid sequence of SEQ ID NO: 4; the anti-CFHR4 HCDR2 comprises the amino acid sequence of SEQ ID NO: 11; and the anti-CFHR4 HCDR3 comprises the amino acid sequence of SEQ ID NO: 17. In some embodiments, the anti-CFHR4 HCDR1 comprises the amino acid sequence of SEQ ID NO: 5; the anti-CFHR4 HCDR2 comprises the amino acid sequence of SEQ ID NO: 12; and the anti-CFHR4 HCDR3 comprises the amino acid sequence of SEQ ID NO: 18. In some embodiments, the anti-CFHR4 HCDR1 comprises the amino acid sequence of SEQ ID NO: 6; the anti-CFHR4 HCDR2 comprises the amino acid sequence of SEQ ID NO: 13; and the anti-CFHR4 HCDR3 comprises the amino acid sequence of SEQ ID NO: 19. In some embodiments, the anti-CFHR4 HCDR1 comprises the amino acid sequence of SEQ ID NO: 7; the anti-CFHR4 HCDR2 comprises the amino acid sequence of SEQ ID NO: 14; and the anti-CFHR4 HCDR3 comprises the amino acid sequence of SEQ ID NO: 20. In some embodiments, the anti-CFHR4 HCDR1 comprises the amino acid sequence of SEQ ID NO: 22; the anti-CFHR4 HCDR2 comprises the amino acid sequence of SEQ ID NO: 33; and the anti-CFHR4 HCDR3 comprises the amino acid sequence of SEQ ID NO: 43. In some embodiments, the anti-CFHR4 HCDR1 comprises the amino acid sequence of SEQ ID NO: 23; the anti-CFHR4 HCDR2 comprises the amino acid sequence of SEQ ID NO: 34; and the anti-CFHR4 HCDR3 comprises the amino acid sequence of SEQ ID NO: 44. In some embodiments, the anti-CFHR4 HCDR1 comprises the amino acid sequence of SEQ ID NO: 24; the anti-CFHR4 HCDR2 comprises the amino acid sequence of SEQ ID NO: 35; and the anti-CFHR4 HCDR3 comprises the amino acid sequence of SEQ ID NO: 45. In some embodiments, the anti-CFHR4 HCDR1 comprises the amino acid sequence of SEQ ID NO: 25; the anti-CFHR4 HCDR2 comprises the amino acid sequence of SEQ ID NO: 36; and the anti-CFHR4 HCDR3 comprises the amino acid sequence of SEQ ID NO: 46. In some embodiments, the anti-CFHR4 HCDR1 comprises the amino acid sequence of SEQ ID NO: 26; the anti-CFHR4 HCDR2 comprises the amino acid sequence of SEQ ID NO: 37; and the anti-CFHR4 HCDR3 comprises the amino acid sequence of SEQ ID NO: 47. In some embodiments, the anti-CFHR4 HCDR1 comprises the amino acid sequence of SEQ ID NO: 27; the anti-CFHR4 HCDR2 comprises the amino acid sequence of SEQ ID NO: 38; and the anti-CFHR4 HCDR3 comprises the amino acid sequence of SEQ ID NO: 48. In some embodiments, the anti-CFHR4 HCDR1 comprises the amino acid sequence of SEQ ID NO: 28; the anti-CFHR4 HCDR2 comprises the amino acid sequence of SEQ ID NO: 39; and the anti-CFHR4 HCDR3 comprises the amino acid sequence of SEQ ID NO: 49. In some embodiments, the anti-CFHR4 HCDR1 comprises the amino acid sequence of SEQ ID NO: 29; the anti-CFHR4 HCDR2 comprises the amino acid sequence of SEQ ID NO: 40; and the anti-CFHR4 HCDR3 comprises the amino acid sequence of SEQ ID NO: 50. In some embodiments, the anti-CFHR4 HCDR1 comprises the amino acid sequence of SEQ ID NO: 30; the anti-CFHR4 HCDR2 comprises the amino acid sequence of SEQ ID NO: 41; and the anti-CFHR4 HCDR3 comprises the amino acid sequence of SEQ ID NO: 51. In some embodiments, the anti-CFHR4 HCDR1 comprises the amino acid sequence of SEQ ID NO: 31; the anti-CFHR4 HCDR2 comprises the amino acid sequence of SEQ ID NO: 42; and the anti-CFHR4 HCDR3 comprises the amino acid sequence of SEQ ID NO: 52. In some embodiments, the anti-CFHR4 HCDR1 comprises the amino acid sequence of SEQ ID NO: 54; the anti-CFHR4 HCDR2 comprises the amino acid sequence of SEQ ID NO: 66; and the anti-CFHR4 HCDR3 comprises the amino acid sequence of SEQ ID NO: 77. In some embodiments, the anti-CFHR4 HCDR1 comprises the amino acid sequence of SEQ ID NO: 55; the anti-CFHR4 HCDR2 comprises the amino acid sequence of SEQ ID NO: 67; and the anti-CFHR4 HCDR3 comprises the amino acid sequence of SEQ ID NO: 78. In some embodiments, the anti-CFHR4 HCDR1 comprises the amino acid sequence of SEQ ID NO: 56; the anti-CFHR4 HCDR2 comprises the amino acid sequence of SEQ ID NO: 68; and the anti-CFHR4 HCDR3 comprises the amino acid sequence of SEQ ID NO: 79. In some embodiments, the anti-CFHR4 HCDR1 comprises the amino acid sequence of SEQ ID NO: 57; the anti-CFHR4 HCDR2 comprises the amino acid sequence of SEQ ID NO: 69; and the anti-CFHR4 HCDR3 comprises the amino acid sequence of SEQ ID NO: 80. In some embodiments, the anti-CFHR4 HCDR1 comprises the amino acid sequence of SEQ ID NO: 58; the anti-CFHR4 HCDR2 comprises the amino acid sequence of SEQ ID NO: 70; and the anti-CFHR4 HCDR3 comprises the amino acid sequence of SEQ ID NO: 81. In some embodiments, the anti-CFHR4 HCDR1 comprises the amino acid sequence of SEQ ID NO: 59; the anti-CFHR4 HCDR2 comprises the amino acid sequence of SEQ ID NO: 71; and the anti-CFHR4 HCDR3 comprises the amino acid sequence of SEQ ID NO: 82. In some embodiments, the anti-CFHR4 HCDR1 comprises the amino acid sequence of SEQ ID NO: 60; the anti-CFHR4 HCDR2 comprises the amino acid sequence of SEQ ID NO: 72; and the anti-CFHR4 HCDR3 comprises the amino acid sequence of SEQ ID NO: 83. In some embodiments, the anti-CFHR4 HCDR1 comprises the amino acid sequence of SEQ ID NO: 61; the anti-CFHR4 HCDR2 comprises the amino acid sequence of SEQ ID NO: 73; and the anti-CFHR4 HCDR3 comprises the amino acid sequence of SEQ ID NO: 84. In some embodiments, the anti-CFHR4 HCDR1 comprises the amino acid sequence of SEQ ID NO: 62; the anti-CFHR4 HCDR2 comprises the amino acid sequence of SEQ ID NO: 74; and the anti-CFHR4 HCDR3 comprises the amino acid sequence of SEQ ID NO: 85. In some embodiments, the anti-CFHR4 HCDR1 comprises the amino acid sequence of SEQ ID NO: 63; the anti-CFHR4 HCDR2 comprises the amino acid sequence of SEQ ID NO: 75; and the anti-CFHR4 HCDR3 comprises the amino acid sequence of SEQ ID NO: 86. In some embodiments, the anti-CFHR4 HCDR1 comprises the amino acid sequence of SEQ ID NO: 64; the anti-CFHR4 HCDR2 comprises the amino acid sequence of SEQ ID NO: 76; and the anti-CFHR4 HCDR3 comprises the amino acid sequence of SEQ ID NO: 87. In some embodiments, the anti-CFHR4 HCDR1 comprises the amino acid sequence of SEQ ID NO: 89; the anti-CFHR4 HCDR2 comprises the amino acid sequence of SEQ ID NO: 103; and the anti-CFHR4 HCDR3 comprises the amino acid sequence of SEQ ID NO: 116. In some embodiments, the anti-CFHR4 HCDR1 comprises the amino acid sequence of SEQ ID NO: 90; the anti-CFHR4 HCDR2 comprises the amino acid sequence of SEQ ID NO: 104; and the anti-CFHR4 HCDR3 comprises the amino acid sequence of SEQ ID NO: 117. In some embodiments, the anti-CFHR4 HCDR1 comprises the amino acid sequence of SEQ ID NO: 91; the anti-CFHR4 HCDR2 comprises the amino acid sequence of SEQ ID NO: 105; and the anti-CFHR4 HCDR3 comprises the amino acid sequence of SEQ ID NO: 118. In some embodiments, the anti-CFHR4 HCDR1 comprises the amino acid sequence of SEQ ID NO: 92; the anti-CFHR4 HCDR2 comprises the amino acid sequence of SEQ ID NO: 106; and the anti-CFHR4 HCDR3 comprises the amino acid sequence of SEQ ID NO: 119. In some embodiments, the anti-CFHR4 HCDR1 comprises the amino acid sequence of SEQ ID NO: 93; the anti-CFHR4 HCDR2 comprises the amino acid sequence of SEQ ID NO: 107; and the anti-CFHR4 HCDR3 comprises the amino acid sequence of SEQ ID NO: 120. In some embodiments, the anti-CFHR4 HCDR1 comprises the amino acid sequence of SEQ ID NO: 94; the anti-CFHR4 HCDR2 comprises the amino acid sequence of SEQ ID NO: 108; and the anti-CFHR4 HCDR3 comprises the amino acid sequence of SEQ ID NO: 121. In some embodiments, the anti-CFHR4 HCDR1 comprises the amino acid sequence of SEQ ID NO: 95; the anti-CFHR4 HCDR2 comprises the amino acid sequence of SEQ ID NO: 109; and the anti-CFHR4 HCDR3 comprises the amino acid sequence of SEQ ID NO: 122. In some embodiments, the anti-CFHR4 HCDR1 comprises the amino acid sequence of SEQ ID NO: 96; the anti-CFHR4 HCDR2 comprises the amino acid sequence of SEQ ID NO: 110; and the anti-CFHR4 HCDR3 comprises the amino acid sequence of SEQ ID NO: 123. In some embodiments, the anti-CFHR4 HCDR1 comprises the amino acid sequence of SEQ ID NO: 97; the anti-CFHR4 HCDR2 comprises the amino acid sequence of SEQ ID NO: 111; and the anti-CFHR4 HCDR3 comprises the amino acid sequence of SEQ ID NO: 124. In some embodiments, the anti-CFHR4 HCDR1 comprises the amino acid sequence of SEQ ID NO: 98; the anti-CFHR4 HCDR2 comprises the amino acid sequence of SEQ ID NO: 112; and the anti-CFHR4 HCDR3 comprises the amino acid sequence of SEQ ID NO: 125. In some embodiments, the anti-CFHR4 HCDR1 comprises the amino acid sequence of SEQ ID NO: 99; the anti-CFHR4 HCDR2 comprises the amino acid sequence of SEQ ID NO: 113; and the HCDR3 comprises the amino acid sequence of SEQ ID NO: 126. In some embodiments, the anti-CFHR4 HCDR1 comprises the amino acid sequence of SEQ ID NO: 100; the HCDR2 comprises the amino acid sequence of SEQ ID NO: 114; and the HCDR3 comprises the amino acid sequence of SEQ ID NO: 127. In some embodiments, the anti-CFHR4 HCDR1 comprises the amino acid sequence of SEQ ID NO: 101; the anti-CFHR4 HCDR2 comprises the amino acid sequence of SEQ ID NO: 115; and the anti-CFHR4 HCDR3 comprises the amino acid sequence of SEQ ID NO: 128.

In some embodiments, the anti-CFHR4 LCDR1 comprises the amino acid sequence of SEQ ID NO: 130; the anti-CFHR4 LCDR2 comprises the amino acid sequence of SEQ ID NO: 153; and the anti-CFHR4 LCDR3 comprises the amino acid sequence of SEQ ID NO: 175. In some embodiments, the anti-CFHR4 LCDR1 comprises the amino acid sequence of SEQ ID NO: 131; the anti-CFHR4 LCDR2 comprises the amino acid sequence of SEQ ID NO: 154; and the anti-CFHR4 LCDR3 comprises the amino acid sequence of SEQ ID NO: 176. In some embodiments, the anti-CFHR4 LCDR1 comprises the amino acid sequence of SEQ ID NO: 132; the anti-CFHR4 LCDR2 comprises the amino acid sequence of SEQ ID NO: 155; and the anti-CFHR4 LCDR3 comprises the amino acid sequence of SEQ ID NO: 177. In some embodiments, the anti-CFHR4 LCDR1 comprises the amino acid sequence of SEQ ID NO: 133; the anti-CFHR4 LCDR2 comprises the amino acid sequence of SEQ ID NO: 156; and the anti-CFHR4 LCDR3 comprises the amino acid sequence of SEQ ID NO: 178. In some embodiments, the anti-CFHR4 LCDR1 comprises the amino acid sequence of SEQ ID NO: 134; the anti-CFHR4 LCDR2 comprises the amino acid sequence of SEQ ID NO: 157; and the anti-CFHR4 LCDR3 comprises the amino acid sequence of SEQ ID NO: 179. In some embodiments, the anti-CFHR4 LCDR1 comprises the amino acid sequence of SEQ ID NO: 135; the anti-CFHR4 LCDR2 comprises the amino acid sequence of SEQ ID NO: 158; and the anti-CFHR4 LCDR3 comprises the amino acid sequence of SEQ ID NO: 180. In some embodiments, the anti-CFHR4 LCDR1 comprises the amino acid sequence of SEQ ID NO: 136; the anti-CFHR4 LCDR2 comprises the amino acid sequence of SEQ ID NO: 159; and the anti-CFHR4 LCDR3 comprises the amino acid sequence of SEQ ID NO: 181. In some embodiments, the anti-CFHR4 LCDR1 comprises the amino acid sequence of SEQ ID NO: 137; the anti-CFHR4 LCDR2 comprises the amino acid sequence of SEQ ID NO: 160; and the anti-CFHR4 LCDR3 comprises the amino acid sequence of SEQ ID NO: 182. In some embodiments, the anti-CFHR4 LCDR1 comprises the amino acid sequence of SEQ ID NO: 138; the anti-CFHR4 LCDR2 comprises the amino acid sequence of SEQ ID NO: 161; and the anti-CFHR4 LCDR3 comprises the amino acid sequence of SEQ ID NO: 183. In some embodiments, the anti-CFHR4 LCDR1 comprises the amino acid sequence of SEQ ID NO: 139; the anti-CFHR4 LCDR2 comprises the amino acid sequence of SEQ ID NO: 162; and the anti-CFHR4 LCDR3 comprises the amino acid sequence of SEQ ID NO: 184. In some embodiments, the anti-CFHR4 LCDR1 comprises the amino acid sequence of SEQ ID NO: 140; the anti-CFHR4 LCDR2 comprises the amino acid sequence of SEQ ID NO: 163; and the anti-CFHR4 LCDR3 comprises the amino acid sequence of SEQ ID NO: 185. In some embodiments, the anti-CFHR4 LCDR1 comprises the amino acid sequence of SEQ ID NO: 141; the anti-CFHR4 LCDR2 comprises the amino acid sequence of SEQ ID NO: 164; and the anti-CFHR4 LCDR3 comprises the amino acid sequence of SEQ ID NO: 186. In some embodiments, the anti-CFHR4 LCDR1 comprises the amino acid sequence of SEQ ID NO: 142; the anti-CFHR4 LCDR2 comprises the amino acid sequence of SEQ ID NO: 165; and the anti-CFHR4 LCDR3 comprises the amino acid sequence of SEQ ID NO: 187. In some embodiments, the anti-CFHR4 LCDR1 comprises the amino acid sequence of SEQ ID NO: 143; the anti-CFHR4 LCDR2 comprises the amino acid sequence of SEQ ID NO: 166; and the anti-CFHR4 LCDR3 comprises the amino acid sequence of SEQ ID NO: 188. In some embodiments, the anti-CFHR4 LCDR1 comprises the amino acid sequence of SEQ ID NO: 144; the anti-CFHR4 LCDR2 comprises the amino acid sequence of SEQ ID NO: 167; and the anti-CFHR4 LCDR3 comprises the amino acid sequence of SEQ ID NO: 189. In some embodiments, the anti-CFHR4 LCDR1 comprises the amino acid sequence of SEQ ID NO: 145; the anti-CFHR4 LCDR2 comprises the amino acid sequence of SEQ ID NO: 168; and the anti-CFHR4 LCDR3 comprises the amino acid sequence of SEQ ID NO: 190. In some embodiments, the anti-CFHR4 LCDR1 comprises the amino acid sequence of SEQ ID NO: 146; the anti-CFHR4 LCDR2 comprises the amino acid sequence of SEQ ID NO: 169; and the anti-CFHR4 LCDR3 comprises the amino acid sequence of SEQ ID NO: 191. In some embodiments, the anti-CFHR4 LCDR1 comprises the amino acid sequence of SEQ ID NO: 147; the anti-CFHR4 LCDR2 comprises the amino acid sequence of SEQ ID NO: 170; and the anti-CFHR4 LCDR3 comprises the amino acid sequence of SEQ ID NO: 192. In some embodiments, the anti-CFHR4 LCDR1 comprises the amino acid sequence of SEQ ID NO: 148; the anti-CFHR4 LCDR2 comprises the amino acid sequence of SEQ ID NO: 171; and the anti-CFHR4 LCDR3 comprises the amino acid sequence of SEQ ID NO: 193. In some embodiments, the anti-CFHR4 LCDR1 comprises the amino acid sequence of SEQ ID NO: 149; the anti-CFHR4 LCDR2 comprises the amino acid sequence of SEQ ID NO: 172; and the anti-CFHR4 LCDR3 comprises the amino acid sequence of SEQ ID NO: 194. In some embodiments, the anti-CFHR4 LCDR1 comprises the amino acid sequence of SEQ ID NO: 150; the anti-CFHR4 LCDR2 comprises the amino acid sequence of SEQ ID NO: 173; and the anti-CFHR4 LCDR3 comprises the amino acid sequence of SEQ ID NO: 195. In some embodiments, the anti-CFHR4 LCDR1 comprises the amino acid sequence of SEQ ID NO: 151; the anti-CFHR4 LCDR2 comprises the amino acid sequence of SEQ ID NO: 174; and the anti-CFHR4 LCDR3 comprises the amino acid sequence of SEQ ID NO: 196. In some embodiments, the anti-CFHR4 LCDR1 comprises the amino acid sequence of SEQ ID NO: 198; the anti-CFHR4 LCDR2 comprises the amino acid sequence of SEQ ID NO: 201; and the anti-CFHR4 LCDR3 comprises the amino acid sequence of SEQ ID NO: 204. In some embodiments, the anti-CFHR4 LCDR1 comprises the amino acid sequence of SEQ ID NO: 199; the anti-CFHR4 LCDR2 comprises the amino acid sequence of SEQ ID NO: 202; and the anti-CFHR4 LCDR3 comprises the amino acid sequence of SEQ ID NO: 205. In some embodiments, the anti-CFHR4 LCDR1 comprises the amino acid sequence of SEQ ID NO: 207; the anti-CFHR4 LCDR2 comprises the amino acid sequence of SEQ ID NO: 217; and the anti-CFHR4 LCDR3 comprises the amino acid sequence of SEQ ID NO: 227. In some embodiments, the anti-CFHR4 LCDR1 comprises the amino acid sequence of SEQ ID NO: 208; the anti-CFHR4 LCDR2 comprises the amino acid sequence of SEQ ID NO: 218; and the anti-CFHR4 LCDR3 comprises the amino acid sequence of SEQ ID NO: 228. In some embodiments, the anti-CFHR4 LCDR1 comprises the amino acid sequence of SEQ ID NO: 209; the anti-CFHR4 LCDR2 comprises the amino acid sequence of SEQ ID NO: 219; and the anti-CFHR4 LCDR3 comprises the amino acid sequence of SEQ ID NO: 229. In some embodiments, the anti-CFHR4 LCDR1 comprises the amino acid sequence of SEQ ID NO: 210; the anti-CFHR4 LCDR2 comprises the amino acid sequence of SEQ ID NO: 220; and the anti-CFHR4 LCDR3 comprises the amino acid sequence of SEQ ID NO: 230. In some embodiments, the anti-CFHR4 LCDR1 comprises the amino acid sequence of SEQ ID NO: 211; the anti-CFHR4 LCDR2 comprises the amino acid sequence of SEQ ID NO: 221; and the anti-CFHR4 LCDR3 comprises the amino acid sequence of SEQ ID NO: 231. In some embodiments, the anti-CFHR4 LCDR1 comprises the amino acid sequence of SEQ ID NO: 212; the anti-CFHR4 LCDR2 comprises the amino acid sequence of SEQ ID NO: 222; and the anti-CFHR4 LCDR3 comprises the amino acid sequence of SEQ ID NO: 232. In some embodiments, the anti-CFHR4 LCDR1 comprises the amino acid sequence of SEQ ID NO: 213; the anti-CFHR4 LCDR2 comprises the amino acid sequence of SEQ ID NO: 223; and the anti-CFHR4 LCDR3 comprises the amino acid sequence of SEQ ID NO: 233. In some embodiments, the anti-CFHR4 LCDR1 comprises the amino acid sequence of SEQ ID NO: 214; the anti-CFHR4 LCDR2 comprises the amino acid sequence of SEQ ID NO: 224; and the anti-CFHR4 LCDR3 comprises the amino acid sequence of SEQ ID NO: 234. In some embodiments, the anti-CFHR4 LCDR1 comprises the amino acid sequence of SEQ ID NO: 215; the anti-CFHR4 LCDR2 comprises the amino acid sequence of SEQ ID NO: 225; and the anti-CFHR4 LCDR3 comprises the amino acid sequence of SEQ ID NO: 235. In some embodiments, the anti-CFHR4 LCDR1 comprises the amino acid sequence of SEQ ID NO: 237; the anti-CFHR4 LCDR2 comprises the amino acid sequence of SEQ ID NO: 245; and the anti-CFHR4 LCDR3 comprises the amino acid sequence of SEQ ID NO: 253. In some embodiments, the anti-CFHR4 LCDR1 comprises the amino acid sequence of SEQ ID NO: 238; the anti-CFHR4 LCDR2 comprises the amino acid sequence of SEQ ID NO: 246; and the anti-CFHR4 LCDR3 comprises the amino acid sequence of SEQ ID NO: 254. In some embodiments, the anti-CFHR4 LCDR1 comprises the amino acid sequence of SEQ ID NO: 239; the anti-CFHR4 LCDR2 comprises the amino acid sequence of SEQ ID NO: 247; and the anti-CFHR4 LCDR3 comprises the amino acid sequence of SEQ ID NO: 255. In some embodiments, the anti-CFHR4 LCDR1 comprises the amino acid sequence of SEQ ID NO: 240; the anti-CFHR4 LCDR2 comprises the amino acid sequence of SEQ ID NO: 248; and the anti-CFHR4 LCDR3 comprises the amino acid sequence of SEQ ID NO: 256. In some embodiments, the anti-CFHR4 LCDR1 comprises the amino acid sequence of SEQ ID NO: 241; the anti-CFHR4 LCDR2 comprises the amino acid sequence of SEQ ID NO: 249; and the anti-CFHR4 LCDR3 comprises the amino acid sequence of SEQ ID NO: 257. In some embodiments, the anti-CFHR4 LCDR1 comprises the amino acid sequence of SEQ ID NO: 242; the anti-CFHR4 LCDR2 comprises the amino acid sequence of SEQ ID NO: 250; and the anti-CFHR4 LCDR3 comprises the amino acid sequence of SEQ ID NO: 258. In some embodiments, the anti-CFHR4 LCDR1 comprises the amino acid sequence of SEQ ID NO: 243; the anti-CFHR4 LCDR2 comprises the amino acid sequence of SEQ ID NO: 251; and the anti-CFHR4 LCDR3 comprises the amino acid sequence of SEQ ID NO: 259.

In some embodiments, the anti-C3 HCDR1 comprises the amino acid sequence of SEQ ID NO: 583; the anti-C3 HCDR2 comprises the amino acid sequence of SEQ ID NO: 602; and the anti-C3 HCDR3 comprises the amino acid sequence of SEQ ID NO: 620. In some embodiments, the anti-C3 HCDR1 comprises the amino acid sequence of SEQ ID NO: 584; the anti-C3 HCDR2 comprises the amino acid sequence of SEQ ID NO: 603; and the anti-C3 HCDR3 comprises the amino acid sequence of SEQ ID NO: 621. In some embodiments, the anti-C3 HCDR1 comprises the amino acid sequence of SEQ ID NO: 585; the anti-C3 HCDR2 comprises the amino acid sequence of SEQ ID NO: 604; and the anti-C3 HCDR3 comprises the amino acid sequence of SEQ ID NO: 622. In some embodiments, the anti-C3 HCDR1 comprises the amino acid sequence of SEQ ID NO: 586; the anti-C3 HCDR2 comprises the amino acid sequence of SEQ ID NO: 605; and the anti-C3 HCDR3 comprises the amino acid sequence of SEQ ID NO: 623. In some embodiments, the anti-C3 HCDR1 comprises the amino acid sequence of SEQ ID NO: 587; the anti-C3 HCDR2 comprises the amino acid sequence of SEQ ID NO: 606; and the anti-C3 HCDR3 comprises the amino acid sequence of SEQ ID NO: 624. In some embodiments, the anti-C3 HCDR1 comprises the amino acid sequence of SEQ ID NO: 588; the anti-C3 HCDR2 comprises the amino acid sequence of SEQ ID NO: 607; and the anti-C3 HCDR3 comprises the amino acid sequence of SEQ ID NO: 625. In some embodiments, the anti-C3 HCDR1 comprises the amino acid sequence of SEQ ID NO: 589; the anti-C3 HCDR2 comprises the amino acid sequence of SEQ ID NO: 608; and the anti-C3 HCDR3 comprises the amino acid sequence of SEQ ID NO: 626. In some embodiments, the anti-C3 HCDR1 comprises the amino acid sequence of SEQ ID NO: 590; the anti-C3 HCDR2 comprises the amino acid sequence of SEQ ID NO: 609; and the anti-C3 HCDR3 comprises the amino acid sequence of SEQ ID NO: 627. In some embodiments, the anti-C3 HCDR1 comprises the amino acid sequence of SEQ ID NO: 591; the anti-C3 HCDR2 comprises the amino acid sequence of SEQ ID NO: 610; and the anti-C3 HCDR3 comprises the amino acid sequence of SEQ ID NO: 628. In some embodiments, the anti-C3 HCDR1 comprises the amino acid sequence of SEQ ID NO: 592; the anti-C3 HCDR2 comprises the amino acid sequence of SEQ ID NO: 611; and the anti-C3 HCDR3 comprises the amino acid sequence of SEQ ID NO: 629. In some embodiments, the anti-C3 HCDR1 comprises the amino acid sequence of SEQ ID NO: 593; the anti-C3 HCDR2 comprises the amino acid sequence of SEQ ID NO: 612; and the anti-C3 HCDR3 comprises the amino acid sequence of SEQ ID NO: 630. In some embodiments, the anti-C3 HCDR1 comprises the amino acid sequence of SEQ ID NO: 594; the anti-C3 HCDR2 comprises the amino acid sequence of SEQ ID NO: 613; and the anti-C3 HCDR3 comprises the amino acid sequence of SEQ ID NO: 631. In some embodiments, the anti-C3 HCDR1 comprises the amino acid sequence of SEQ ID NO: 595; the anti-C3 HCDR2 comprises the amino acid sequence of SEQ ID NO: 614; and the anti-C3 HCDR3 comprises the amino acid sequence of SEQ ID NO: 632. In some embodiments, the anti-C3 HCDR1 comprises the amino acid sequence of SEQ ID NO: 596; the anti-C3 HCDR2 comprises the amino acid sequence of SEQ ID NO: 615; and the anti-C3 HCDR3 comprises the amino acid sequence of SEQ ID NO: 633. In some embodiments, the anti-C3 HCDR1 comprises the amino acid sequence of SEQ ID NO: 597; the anti-C3 HCDR2 comprises the amino acid sequence of SEQ ID NO: 616; and the anti-C3 HCDR3 comprises the amino acid sequence of SEQ ID NO: 634. In some embodiments, the anti-C3 HCDR1 comprises the amino acid sequence of SEQ ID NO: 598; the anti-C3 HCDR2 comprises the amino acid sequence of SEQ ID NO: 617; and the anti-C3 HCDR3 comprises the amino acid sequence of SEQ ID NO: 635. In some embodiments, the anti-C3 HCDR1 comprises the amino acid sequence of SEQ ID NO: 599; the anti-C3 HCDR2 comprises the amino acid sequence of SEQ ID NO: 618; and the anti-C3 HCDR3 comprises the amino acid sequence of SEQ ID NO: 636. In some embodiments, the anti-C3 HCDR1 comprises the amino acid sequence of SEQ ID NO: 600; the anti-C3 HCDR2 comprises the amino acid sequence of SEQ ID NO: 619; and the anti-C3 HCDR3 comprises the amino acid sequence of SEQ ID NO: 637. In some embodiments, the anti-C3 HCDR1 comprises the amino acid sequence of SEQ ID NO: 639; the anti-C3 HCDR2 comprises the amino acid sequence of SEQ ID NO: 652; and the anti-C3 HCDR3 comprises the amino acid sequence of SEQ ID NO: 664. In some embodiments, the anti-C3 HCDR1 comprises the amino acid sequence of SEQ ID NO: 640; the anti-C3 HCDR2 comprises the amino acid sequence of SEQ ID NO: 653; and the anti-C3 HCDR3 comprises the amino acid sequence of SEQ ID NO: 665. In some embodiments, the anti-C3 HCDR1 comprises the amino acid sequence of SEQ ID NO: 641; the anti-C3 HCDR2 comprises the amino acid sequence of SEQ ID NO: 654; and the anti-C3 HCDR3 comprises the amino acid sequence of SEQ ID NO: 666. In some embodiments, the anti-C3 HCDR1 comprises the amino acid sequence of SEQ ID NO: 642; the anti-C3 HCDR2 comprises the amino acid sequence of SEQ ID NO: 655; and the anti-C3 HCDR3 comprises the amino acid sequence of SEQ ID NO: 667. In some embodiments, the anti-C3 HCDR1 comprises the amino acid sequence of SEQ ID NO: 643; the anti-C3 HCDR2 comprises the amino acid sequence of SEQ ID NO: 656; and the anti-C3 HCDR3 comprises the amino acid sequence of SEQ ID NO: 668. In some embodiments, the anti-C3 HCDR1 comprises the amino acid sequence of SEQ ID NO: 644; the anti-C3 HCDR2 comprises the amino acid sequence of SEQ ID NO: 657; and the anti-C3 HCDR3 comprises the amino acid sequence of SEQ ID NO: 669. In some embodiments, the anti-C3 HCDR1 comprises the amino acid sequence of SEQ ID NO: 645; the anti-C3 HCDR2 comprises the amino acid sequence of SEQ ID NO: 658; and the anti-C3 HCDR3 comprises the amino acid sequence of SEQ ID NO: 670. In some embodiments, the anti-C3 HCDR1 comprises the amino acid sequence of SEQ ID NO: 646; the anti-C3 HCDR2 comprises the amino acid sequence of SEQ ID NO: 659; and the anti-C3 HCDR3 comprises the amino acid sequence of SEQ ID NO: 671. In some embodiments, the anti-C3 HCDR1 comprises the amino acid sequence of SEQ ID NO: 647; the anti-C3 HCDR2 comprises the amino acid sequence of SEQ ID NO: 660; and the anti-C3 HCDR3 comprises the amino acid sequence of SEQ ID NO: 672. In some embodiments, the anti-C3 HCDR1 comprises the amino acid sequence of SEQ ID NO: 648; the anti-C3 HCDR2 comprises the amino acid sequence of SEQ ID NO: 661; and the anti-C3 HCDR3 comprises the amino acid sequence of SEQ ID NO: 673. In some embodiments, the anti-C3 HCDR1 comprises the amino acid sequence of SEQ ID NO: 649; the anti-C3 HCDR2 comprises the amino acid sequence of SEQ ID NO: 662; and the anti-C3 HCDR3 comprises the amino acid sequence of SEQ ID NO: 674. In some embodiments, the anti-C3 HCDR1 comprises the amino acid sequence of SEQ ID NO: 650; the anti-C3 HCDR2 comprises the amino acid sequence of SEQ ID NO: 663; and the anti-C3 HCDR3 comprises the amino acid sequence of SEQ ID NO: 675. In some embodiments, the anti-C3 HCDR1 comprises the amino acid sequence of SEQ ID NO: 677; the anti-C3 HCDR2 comprises the amino acid sequence of SEQ ID NO: 706; and the anti-C3 HCDR3 comprises the amino acid sequence of SEQ ID NO: 734. In some embodiments, the anti-C3 HCDR1 comprises the amino acid sequence of SEQ ID NO: 678; the anti-C3 HCDR2 comprises the amino acid sequence of SEQ ID NO: 707; and the anti-C3 HCDR3 comprises the amino acid sequence of SEQ ID NO: 735. In some embodiments, the anti-C3 HCDR1 comprises the amino acid sequence of SEQ ID NO: 679; the anti-C3 HCDR2 comprises the amino acid sequence of SEQ ID NO: 708; and the anti-C3 HCDR3 comprises the amino acid sequence of SEQ ID NO: 736. In some embodiments, the anti-C3 HCDR1 comprises the amino acid sequence of SEQ ID NO: 680; the anti-C3 HCDR2 comprises the amino acid sequence of SEQ ID NO: 709; and the anti-C3 HCDR3 comprises the amino acid sequence of SEQ ID NO: 737. In some embodiments, the anti-C3 HCDR1 comprises the amino acid sequence of SEQ ID NO: 681; the anti-C3 HCDR2 comprises the amino acid sequence of SEQ ID NO: 710; and the anti-C3 HCDR3 comprises the amino acid sequence of SEQ ID NO: 738. In some embodiments, the anti-C3 HCDR1 comprises the amino acid sequence of SEQ ID NO: 682; the anti-C3 HCDR2 comprises the amino acid sequence of SEQ ID NO: 711; and the anti-C3 HCDR3 comprises the amino acid sequence of SEQ ID NO: 739. In some embodiments, the anti-C3 HCDR1 comprises the amino acid sequence of SEQ ID NO: 683; the anti-C3 HCDR2 comprises the amino acid sequence of SEQ ID NO: 712; and the anti-C3 HCDR3 comprises the amino acid sequence of SEQ ID NO: 740. In some embodiments, the anti-C3 HCDR1 comprises the amino acid sequence of SEQ ID NO: 684; the anti-C3 HCDR2 comprises the amino acid sequence of SEQ ID NO: 713; and the anti-C3 HCDR3 comprises the amino acid sequence of SEQ ID NO: 741. In some embodiments, the anti-C3 HCDR1 comprises the amino acid sequence of SEQ ID NO: 685; the anti-C3 HCDR2 comprises the amino acid sequence of SEQ ID NO: 714; and the anti-C3 HCDR3 comprises the amino acid sequence of SEQ ID NO: 742. In some embodiments, the anti-C3 HCDR1 comprises the amino acid sequence of SEQ ID NO: 686; the anti-C3 HCDR2 comprises the amino acid sequence of SEQ ID NO: 715; and the anti-C3 HCDR3 comprises the amino acid sequence of SEQ ID NO: 743. In some embodiments, the anti-C3 HCDR1 comprises the amino acid sequence of SEQ ID NO: 687; the anti-C3 HCDR2 comprises the amino acid sequence of SEQ ID NO: 716; and the anti-C3 HCDR3 comprises the amino acid sequence of SEQ ID NO: 744. In some embodiments, the anti-C3 HCDR1 comprises the amino acid sequence of SEQ ID NO: 688; the anti-C3 HCDR2 comprises the amino acid sequence of SEQ ID NO: 717; and the anti-C3 HCDR3 comprises the amino acid sequence of SEQ ID NO: 745. In some embodiments, the anti-C3 HCDR1 comprises the amino acid sequence of SEQ ID NO: 689; the anti-C3 HCDR2 comprises the amino acid sequence of SEQ ID NO: 718; and the anti-C3 HCDR3 comprises the amino acid sequence of SEQ ID NO: 746. In some embodiments, the anti-C3 HCDR1 comprises the amino acid sequence of SEQ ID NO: 690; the anti-C3 HCDR2 comprises the amino acid sequence of SEQ ID NO: 719; and the anti-C3 HCDR3 comprises the amino acid sequence of SEQ ID NO: 747. In some embodiments, the anti-C3 HCDR1 comprises the amino acid sequence of SEQ ID NO: 691; the anti-C3 HCDR2 comprises the amino acid sequence of SEQ ID NO: 720; and the anti-C3 HCDR3 comprises the amino acid sequence of SEQ ID NO: 748. In some embodiments, the anti-C3 HCDR1 comprises the amino acid sequence of SEQ ID NO: 692; the anti-C3 HCDR2 comprises the amino acid sequence of SEQ ID NO: 721; and the anti-C3 HCDR3 comprises the amino acid sequence of SEQ ID NO: 749. In some embodiments, the anti-C3 HCDR1 comprises the amino acid sequence of SEQ ID NO: 693; the anti-C3 HCDR2 comprises the amino acid sequence of SEQ ID NO: 722; and the anti-C3 HCDR3 comprises the amino acid sequence of SEQ ID NO: 750. In some embodiments, the anti-C3 HCDR1 comprises the amino acid sequence of SEQ ID NO: 694; the anti-C3 HCDR2 comprises the amino acid sequence of SEQ ID NO: 723; and the anti-C3 HCDR3 comprises the amino acid sequence of SEQ ID NO: 751. In some embodiments, the anti-C3 HCDR1 comprises the amino acid sequence of SEQ ID NO: 695; the anti-C3 HCDR2 comprises the amino acid sequence of SEQ ID NO: 724; and the anti-C3 HCDR3 comprises the amino acid sequence of SEQ ID NO: 752. In some embodiments, the anti-C3 HCDR1 comprises the amino acid sequence of SEQ ID NO: 696; the anti-C3 HCDR2 comprises the amino acid sequence of SEQ ID NO: 725; and the anti-C3 HCDR3 comprises the amino acid sequence of SEQ ID NO: 753. In some embodiments, the anti-C3 HCDR1 comprises the amino acid sequence of SEQ ID NO: 697; the anti-C3 HCDR2 comprises the amino acid sequence of SEQ ID NO: 726; and the anti-C3 HCDR3 comprises the amino acid sequence of SEQ ID NO: 754. In some embodiments, the anti-C3 HCDR1 comprises the amino acid sequence of SEQ ID NO: 698; the anti-C3 HCDR2 comprises the amino acid sequence of SEQ ID NO: 727; and the anti-C3 HCDR3 comprises the amino acid sequence of SEQ ID NO: 755. In some embodiments, the anti-C3 HCDR1 comprises the amino acid sequence of SEQ ID NO: 699; the anti-C3 HCDR2 comprises the amino acid sequence of SEQ ID NO: 728; and the anti-C3 HCDR3 comprises the amino acid sequence of SEQ ID NO: 756. In some embodiments, the anti-C3 HCDR1 comprises the amino acid sequence of SEQ ID NO: 700; the anti-C3 HCDR2 comprises the amino acid sequence of SEQ ID NO: 729; and the anti-C3 HCDR3 comprises the amino acid sequence of SEQ ID NO: 757. In some embodiments, the anti-C3 HCDR1 comprises the amino acid sequence of SEQ ID NO: 701; the anti-C3 HCDR2 comprises the amino acid sequence of SEQ ID NO: 730; and the anti-C3 HCDR3 comprises the amino acid sequence of SEQ ID NO: 758. In some embodiments, the anti-C3 HCDR1 comprises the amino acid sequence of SEQ ID NO: 702; the anti-C3 HCDR2 comprises the amino acid sequence of SEQ ID NO: 731; and the anti-C3 HCDR3 comprises the amino acid sequence of SEQ ID NO: 759. In some embodiments, the anti-C3 HCDR1 comprises the amino acid sequence of SEQ ID NO: 703; the anti-C3 HCDR2 comprises the amino acid sequence of SEQ ID NO: 732; and the anti-C3 HCDR3 comprises the amino acid sequence of SEQ ID NO: 760. In some embodiments, the anti-C3 HCDR1 comprises the amino acid sequence of SEQ ID NO: 704; the anti-C3 HCDR2 comprises the amino acid sequence of SEQ ID NO: 733; and the anti-C3 HCDR3 comprises the amino acid sequence of SEQ ID NO: 761. In some embodiments, the anti-C3 HCDR1 comprises the amino acid sequence of SEQ ID NO: 763; the anti-C3 HCDR2 comprises the amino acid sequence of SEQ ID NO: 771; and the anti-C3 HCDR3 comprises the amino acid sequence of SEQ ID NO: 778. In some embodiments, the anti-C3 HCDR1 comprises the amino acid sequence of SEQ ID NO: 764; the anti-C3 HCDR2 comprises the amino acid sequence of SEQ ID NO: 772; and the anti-C3 HCDR3 comprises the amino acid sequence of SEQ ID NO: 779. In some embodiments, the anti-C3 HCDR1 comprises the amino acid sequence of SEQ ID NO: 765; the anti-C3 HCDR2 comprises the amino acid sequence of SEQ ID NO: 773; and the anti-C3 HCDR3 comprises the amino acid sequence of SEQ ID NO: 780. In some embodiments, the anti-C3 HCDR1 comprises the amino acid sequence of SEQ ID NO: 766; the anti-C3 HCDR2 comprises the amino acid sequence of SEQ ID NO: 774; and the anti-C3 HCDR3 comprises the amino acid sequence of SEQ ID NO: 781. In some embodiments, the anti-C3 HCDR1 comprises the amino acid sequence of SEQ ID NO: 767; the anti-C3 HCDR2 comprises the amino acid sequence of SEQ ID NO: 775; and the anti-C3 HCDR3 comprises the amino acid sequence of SEQ ID NO: 782. In some embodiments, the anti-C3 HCDR1 comprises the amino acid sequence of SEQ ID NO: 768; the anti-C3 HCDR2 comprises the amino acid sequence of SEQ ID NO: 776; and the anti-C3 HCDR3 comprises the amino acid sequence of SEQ ID NO: 783. In some embodiments, the anti-C3 HCDR1 comprises the amino acid sequence of SEQ ID NO: 769; the anti-C3 HCDR2 comprises the amino acid sequence of SEQ ID NO: 777; and the anti-C3 HCDR3 comprises the amino acid sequence of SEQ ID NO: 784. In some embodiments, the anti-C3 HCDR1 comprises the amino acid sequence of SEQ ID NO: 786; the anti-C3 HCDR2 comprises the amino acid sequence of SEQ ID NO: 794; and the anti-C3 HCDR3 comprises the amino acid sequence of SEQ ID NO: 801. In some embodiments, the anti-C3 HCDR1 comprises the amino acid sequence of SEQ ID NO: 787; the anti-C3 HCDR2 comprises the amino acid sequence of SEQ ID NO: 795; and the anti-C3 HCDR3 comprises the amino acid sequence of SEQ ID NO: 802. In some embodiments, the anti-C3 HCDR1 comprises the amino acid sequence of SEQ ID NO: 788; the anti-C3 HCDR2 comprises the amino acid sequence of SEQ ID NO: 796; and the anti-C3 HCDR3 comprises the amino acid sequence of SEQ ID NO: 803. In some embodiments, the anti-C3 HCDR1 comprises the amino acid sequence of SEQ ID NO: 789; the anti-C3 HCDR2 comprises the amino acid sequence of SEQ ID NO: 797; and the anti-C3 HCDR3 comprises the amino acid sequence of SEQ ID NO: 804. In some embodiments, the anti-C3 HCDR1 comprises the amino acid sequence of SEQ ID NO: 790; the anti-C3 HCDR2 comprises the amino acid sequence of SEQ ID NO: 798; and the anti-C3 HCDR3 comprises the amino acid sequence of SEQ ID NO: 805. In some embodiments, the anti-C3 HCDR1 comprises the amino acid sequence of SEQ ID NO: 791; the anti-C3 HCDR2 comprises the amino acid sequence of SEQ ID NO: 799; and the anti-C3 HCDR3 comprises the amino acid sequence of SEQ ID NO: 806. In some embodiments, the anti-C3 HCDR1 comprises the amino acid sequence of SEQ ID NO: 792; the anti-C3 HCDR2 comprises the amino acid sequence of SEQ ID NO: 800; and the anti-C3 HCDR3 comprises the amino acid sequence of SEQ ID NO: 807. In some embodiments, the anti-C3 HCDR1 comprises the amino acid sequence of SEQ ID NO: 809; the anti-C3 HCDR2 comprises the amino acid sequence of SEQ ID NO: 812; and the anti-C3 HCDR3 comprises the amino acid sequence of SEQ ID NO: 815. In some embodiments, the anti-C3 HCDR1 comprises the amino acid sequence of SEQ ID NO: 810; the anti-C3 HCDR2 comprises the amino acid sequence of SEQ ID NO: 813; and the anti-C3 HCDR3 comprises the amino acid sequence of SEQ ID NO: 816.

In some embodiments, the anti-C3 LCDR1 comprises the amino acid sequence of SEQ ID NO: 818; the anti-C3 LCDR2 comprises the amino acid sequence of SEQ ID NO: 850; and the anti-C3 LCDR3 comprises the amino acid sequence of SEQ ID NO: 881. In some embodiments, the anti-C3 LCDR1 comprises the amino acid sequence of SEQ ID NO: 819; the anti-C3 LCDR2 comprises the amino acid sequence of SEQ ID NO: 851; and the anti-C3 LCDR3 comprises the amino acid sequence of SEQ ID NO: 882. In some embodiments, the anti-C3 LCDR1 comprises the amino acid sequence of SEQ ID NO: 820; the anti-C3 LCDR2 comprises the amino acid sequence of SEQ ID NO: 852; and the anti-C3 LCDR3 comprises the amino acid sequence of SEQ ID NO: 883. In some embodiments, the anti-C3 LCDR1 comprises the amino acid sequence of SEQ ID NO: 821; the anti-C3 LCDR2 comprises the amino acid sequence of SEQ ID NO: 853; and the anti-C3 LCDR3 comprises the amino acid sequence of SEQ ID NO: 884. In some embodiments, the anti-C3 LCDR1 comprises the amino acid sequence of SEQ ID NO: 822; the anti-C3 LCDR2 comprises the amino acid sequence of SEQ ID NO: 854; and the anti-C3 LCDR3 comprises the amino acid sequence of SEQ ID NO: 885. In some embodiments, the anti-C3 LCDR1 comprises the amino acid sequence of SEQ ID NO: 823; the anti-C3 LCDR2 comprises the amino acid sequence of SEQ ID NO: 855; and the anti-C3 LCDR3 comprises the amino acid sequence of SEQ ID NO: 886. In some embodiments, the anti-C3 LCDR1 comprises the amino acid sequence of SEQ ID NO: 824; the anti-C3 LCDR2 comprises the amino acid sequence of SEQ ID NO: 856; and the anti-C3 LCDR3 comprises the amino acid sequence of SEQ ID NO: 887. In some embodiments, the anti-C3 LCDR1 comprises the amino acid sequence of SEQ ID NO: 825; the anti-C3 LCDR2 comprises the amino acid sequence of SEQ ID NO: 857; and the anti-C3 LCDR3 comprises the amino acid sequence of SEQ ID NO: 888. In some embodiments, the anti-C3 LCDR1 comprises the amino acid sequence of SEQ ID NO: 826; the anti-C3 LCDR2 comprises the amino acid sequence of SEQ ID NO: 858; and the anti-C3 LCDR3 comprises the amino acid sequence of SEQ ID NO: 889. In some embodiments, the anti-C3 LCDR1 comprises the amino acid sequence of SEQ ID NO: 827; the anti-C3 LCDR2 comprises the amino acid sequence of SEQ ID NO: 859; and the anti-C3 LCDR3 comprises the amino acid sequence of SEQ ID NO: 890. In some embodiments, the anti-C3 LCDR1 comprises the amino acid sequence of SEQ ID NO: 828; the anti-C3 LCDR2 comprises the amino acid sequence of SEQ ID NO: 860; and the anti-C3 LCDR3 comprises the amino acid sequence of SEQ ID NO: 891. In some embodiments, the anti-C3 LCDR1 comprises the amino acid sequence of SEQ ID NO: 829; the anti-C3 LCDR2 comprises the amino acid sequence of SEQ ID NO: 861; and the anti-C3 LCDR3 comprises the amino acid sequence of SEQ ID NO: 892. In some embodiments, the anti-C3 LCDR1 comprises the amino acid sequence of SEQ ID NO: 830; the anti-C3 LCDR2 comprises the amino acid sequence of SEQ ID NO: 862; and the anti-C3 LCDR3 comprises the amino acid sequence of SEQ ID NO: 893. In some embodiments, the anti-C3 LCDR1 comprises the amino acid sequence of SEQ ID NO: 831; the anti-C3 LCDR2 comprises the amino acid sequence of SEQ ID NO: 863; and the anti-C3 LCDR3 comprises the amino acid sequence of SEQ ID NO: 894. In some embodiments, the anti-C3 LCDR1 comprises the amino acid sequence of SEQ ID NO: 832; the anti-C3 LCDR2 comprises the amino acid sequence of SEQ ID NO: 864; and the anti-C3 LCDR3 comprises the amino acid sequence of SEQ ID NO: 895. In some embodiments, the anti-C3 LCDR1 comprises the amino acid sequence of SEQ ID NO: 833; the anti-C3 LCDR2 comprises the amino acid sequence of SEQ ID NO: 865; and the anti-C3 LCDR3 comprises the amino acid sequence of SEQ ID NO: 896. In some embodiments, the anti-C3 LCDR1 comprises the amino acid sequence of SEQ ID NO: 834; the anti-C3 LCDR2 comprises the amino acid sequence of SEQ ID NO: 866; and the anti-C3 LCDR3 comprises the amino acid sequence of SEQ ID NO: 897. In some embodiments, the anti-C3 LCDR1 comprises the amino acid sequence of SEQ ID NO: 835; the anti-C3 LCDR2 comprises the amino acid sequence of SEQ ID NO: 867; and the anti-C3 LCDR3 comprises the amino acid sequence of SEQ ID NO: 898. In some embodiments, the anti-C3 LCDR1 comprises the amino acid sequence of SEQ ID NO: 836; the anti-C3 LCDR2 comprises the amino acid sequence of SEQ ID NO: 868; and the anti-C3 LCDR3 comprises the amino acid sequence of SEQ ID NO: 899. In some embodiments, the anti-C3 LCDR1 comprises the amino acid sequence of SEQ ID NO: 837; the anti-C3 LCDR2 comprises the amino acid sequence of SEQ ID NO: 869; and the anti-C3 LCDR3 comprises the amino acid sequence of SEQ ID NO: 900. In some embodiments, the anti-C3 LCDR1 comprises the amino acid sequence of SEQ ID NO: 838; the anti-C3 LCDR2 comprises the amino acid sequence of SEQ ID NO: 870; and the anti-C3 LCDR3 comprises the amino acid sequence of SEQ ID NO: 901. In some embodiments, the anti-C3 LCDR1 comprises the amino acid sequence of SEQ ID NO: 839; the anti-C3 LCDR2 comprises the amino acid sequence of SEQ ID NO: 871; and the anti-C3 LCDR3 comprises the amino acid sequence of SEQ ID NO: 902. In some embodiments, the anti-C3 LCDR1 comprises the amino acid sequence of SEQ ID NO: 840; the anti-C3 LCDR2 comprises the amino acid sequence of SEQ ID NO: 872; and the anti-C3 LCDR3 comprises the amino acid sequence of SEQ ID NO: 903. In some embodiments, the anti-C3 LCDR1 comprises the amino acid sequence of SEQ ID NO: 841; the anti-C3 LCDR2 comprises the amino acid sequence of SEQ ID NO: 873; and the anti-C3 LCDR3 comprises the amino acid sequence of SEQ ID NO: 904. In some embodiments, the anti-C3 LCDR1 comprises the amino acid sequence of SEQ ID NO: 842; the anti-C3 LCDR2 comprises the amino acid sequence of SEQ ID NO: 874; and the anti-C3 LCDR3 comprises the amino acid sequence of SEQ ID NO: 905. In some embodiments, the anti-C3 LCDR1 comprises the amino acid sequence of SEQ ID NO: 843; the anti-C3 LCDR2 comprises the amino acid sequence of SEQ ID NO: 875; and the anti-C3 LCDR3 comprises the amino acid sequence of SEQ ID NO: 906. In some embodiments, the anti-C3 LCDR1 comprises the amino acid sequence of SEQ ID NO: 844; the anti-C3 LCDR2 comprises the amino acid sequence of SEQ ID NO: 876; and the anti-C3 LCDR3 comprises the amino acid sequence of SEQ ID NO: 907. In some embodiments, the anti-C3 LCDR1 comprises the amino acid sequence of SEQ ID NO: 845; the anti-C3 LCDR2 comprises the amino acid sequence of SEQ ID NO: 877; and the anti-C3 LCDR3 comprises the amino acid sequence of SEQ ID NO: 908. In some embodiments, the anti-C3 LCDR1 comprises the amino acid sequence of SEQ ID NO: 846; the anti-C3 LCDR2 comprises the amino acid sequence of SEQ ID NO: 878; and the anti-C3 LCDR3 comprises the amino acid sequence of SEQ ID NO: 909. In some embodiments, the anti-C3 LCDR1 comprises the amino acid sequence of SEQ ID NO: 847; the anti-C3 LCDR2 comprises the amino acid sequence of SEQ ID NO: 879; and the anti-C3 LCDR3 comprises the amino acid sequence of SEQ ID NO: 910. In some embodiments, the anti-C3 LCDR1 comprises the amino acid sequence of SEQ ID NO: 848; the anti-C3 LCDR2 comprises the amino acid sequence of SEQ ID NO: 880; and the anti-C3 LCDR3 comprises the amino acid sequence of SEQ ID NO: 911. In some embodiments, the anti-C3 LCDR1 comprises the amino acid sequence of SEQ ID NO: 913; the anti-C3 LCDR2 comprises the amino acid sequence of SEQ ID NO: 916; and the anti-C3 LCDR3 comprises the amino acid sequence of SEQ ID NO: 919. In some embodiments, the anti-C3 LCDR1 comprises the amino acid sequence of SEQ ID NO: 914; the anti-C3 LCDR2 comprises the amino acid sequence of SEQ ID NO: 917; and the anti-C3 LCDR3 comprises the amino acid sequence of SEQ ID NO: 920. In some embodiments, the anti-C3 LCDR1 comprises the amino acid sequence of SEQ ID NO: 914; the anti-C3 LCDR2 comprises the amino acid sequence of SEQ ID NO: 917; and the anti-C3 LCDR3 comprises the amino acid sequence of SEQ ID NO: 920. In some embodiments, the anti-C3 LCDR1 comprises the amino acid sequence of SEQ ID NO: 922; the anti-C3 LCDR2 comprises the amino acid sequence of SEQ ID NO: 931; and the anti-C3 LCDR3 comprises the amino acid sequence of SEQ ID NO: 940. In some embodiments, the anti-C3 LCDR1 comprises the amino acid sequence of SEQ ID NO: 923; the anti-C3 LCDR2 comprises the amino acid sequence of SEQ ID NO: 932; and the anti-C3 LCDR3 comprises the amino acid sequence of SEQ ID NO: 941. In some embodiments, the anti-C3 LCDR1 comprises the amino acid sequence of SEQ ID NO: 924; the anti-C3 LCDR2 comprises the amino acid sequence of SEQ ID NO: 933; and the anti-C3 LCDR3 comprises the amino acid sequence of SEQ ID NO: 942. In some embodiments, the anti-C3 LCDR1 comprises the amino acid sequence of SEQ ID NO: 925; the anti-C3 LCDR2 comprises the amino acid sequence of SEQ ID NO: 934; and the anti-C3 LCDR3 comprises the amino acid sequence of SEQ ID NO: 943. In some embodiments, the anti-C3 LCDR1 comprises the amino acid sequence of SEQ ID NO: 926; the anti-C3 LCDR2 comprises the amino acid sequence of SEQ ID NO: 935; and the anti-C3 LCDR3 comprises the amino acid sequence of SEQ ID NO: 944. In some embodiments, the anti-C3 LCDR1 comprises the amino acid sequence of SEQ ID NO: 927; the anti-C3 LCDR2 comprises the amino acid sequence of SEQ ID NO: 936; and the anti-C3 LCDR3 comprises the amino acid sequence of SEQ ID NO: 945. In some embodiments, the anti-C3 LCDR1 comprises the amino acid sequence of SEQ ID NO: 928; the anti-C3 LCDR2 comprises the amino acid sequence of SEQ ID NO: 937; and the anti-C3 LCDR3 comprises the amino acid sequence of SEQ ID NO: 946. In some embodiments, the anti-C3 LCDR1 comprises the amino acid sequence of SEQ ID NO: 929; the anti-C3 LCDR2 comprises the amino acid sequence of SEQ ID NO: 938; and the anti-C3 LCDR3 comprises the amino acid sequence of SEQ ID NO: 947. In some embodiments, the anti-C3 LCDR1 comprises the amino acid sequence of SEQ ID NO: 949; the anti-C3 LCDR2 comprises the amino acid sequence of SEQ ID NO: 984; and the anti-C3 LCDR3 comprises the amino acid sequence of SEQ ID NO: 1019. In some embodiments, the anti-C3 LCDR1 comprises the amino acid sequence of SEQ ID NO: 950; the anti-C3 LCDR2 comprises the amino acid sequence of SEQ ID NO: 985; and the anti-C3 LCDR3 comprises the amino acid sequence of SEQ ID NO: 1020. In some embodiments, the anti-C3 LCDR1 comprises the amino acid sequence of SEQ ID NO: 951; the anti-C3 LCDR2 comprises the amino acid sequence of SEQ ID NO: 986; and the anti-C3 LCDR3 comprises the amino acid sequence of SEQ ID NO: 1021. In some embodiments, the anti-C3 LCDR1 comprises the amino acid sequence of SEQ ID NO: 952; the anti-C3 LCDR2 comprises the amino acid sequence of SEQ ID NO: 987; and the anti-C3 LCDR3 comprises the amino acid sequence of SEQ ID NO: 1022. In some embodiments, the anti-C3 LCDR1 comprises the amino acid sequence of SEQ ID NO: 953; the anti-C3 LCDR2 comprises the amino acid sequence of SEQ ID NO: 988; and the anti-C3 LCDR3 comprises the amino acid sequence of SEQ ID NO: 1023. In some embodiments, the anti-C3 LCDR1 comprises the amino acid sequence of SEQ ID NO: 954; the anti-C3 LCDR2 comprises the amino acid sequence of SEQ ID NO: 989; and the anti-C3 LCDR3 comprises the amino acid sequence of SEQ ID NO: 1024. In some embodiments, the anti-C3 LCDR1 comprises the amino acid sequence of SEQ ID NO: 955; the anti-C3 LCDR2 comprises the amino acid sequence of SEQ ID NO: 990; and the anti-C3 LCDR3 comprises the amino acid sequence of SEQ ID NO: 1025. In some embodiments, the anti-C3 LCDR1 comprises the amino acid sequence of SEQ ID NO: 956; the anti-C3 LCDR2 comprises the amino acid sequence of SEQ ID NO: 991; and the anti-C3 LCDR3 comprises the amino acid sequence of SEQ ID NO: 1026. In some embodiments, the anti-C3 LCDR1 comprises the amino acid sequence of SEQ ID NO: 957; the anti-C3 LCDR2 comprises the amino acid sequence of SEQ ID NO: 992; and the anti-C3 LCDR3 comprises the amino acid sequence of SEQ ID NO: 1027. In some embodiments, the anti-C3 LCDR1 comprises the amino acid sequence of SEQ ID NO: 958; the anti-C3 LCDR2 comprises the amino acid sequence of SEQ ID NO: 993; and the anti-C3 LCDR3 comprises the amino acid sequence of SEQ ID NO: 1028. In some embodiments, the anti-C3 LCDR1 comprises the amino acid sequence of SEQ ID NO: 959; the anti-C3 LCDR2 comprises the amino acid sequence of SEQ ID NO: 994; and the anti-C3 LCDR3 comprises the amino acid sequence of SEQ ID NO: 1029. In some embodiments, the anti-C3 LCDR1 comprises the amino acid sequence of SEQ ID NO: 960; the anti-C3 LCDR2 comprises the amino acid sequence of SEQ ID NO: 995; and the anti-C3 LCDR3 comprises the amino acid sequence of SEQ ID NO: 1030. In some embodiments, the anti-C3 LCDR1 comprises the amino acid sequence of SEQ ID NO: 961; the anti-C3 LCDR2 comprises the amino acid sequence of SEQ ID NO: 996; and the anti-C3 LCDR3 comprises the amino acid sequence of SEQ ID NO: 1031. In some embodiments, the anti-C3 LCDR1 comprises the amino acid sequence of SEQ ID NO: 962; the anti-C3 LCDR2 comprises the amino acid sequence of SEQ ID NO: 997; and the anti-C3 LCDR3 comprises the amino acid sequence of SEQ ID NO: 1032. In some embodiments, the anti-C3 LCDR1 comprises the amino acid sequence of SEQ ID NO: 963; the anti-C3 LCDR2 comprises the amino acid sequence of SEQ ID NO: 998; and the anti-C3 LCDR3 comprises the amino acid sequence of SEQ ID NO: 1033. In some embodiments, the anti-C3 LCDR1 comprises the amino acid sequence of SEQ ID NO: 964; the anti-C3 LCDR2 comprises the amino acid sequence of SEQ ID NO: 999; and the anti-C3 LCDR3 comprises the amino acid sequence of SEQ ID NO: 1034. In some embodiments, the anti-C3 LCDR1 comprises the amino acid sequence of SEQ ID NO: 965; the anti-C3 LCDR2 comprises the amino acid sequence of SEQ ID NO: 1000; and the anti-C3 LCDR3 comprises the amino acid sequence of SEQ ID NO: 1035. In some embodiments, the anti-C3 LCDR1 comprises the amino acid sequence of SEQ ID NO: 966; the anti-C3 LCDR2 comprises the amino acid sequence of SEQ ID NO: 1001; and the anti-C3 LCDR3 comprises the amino acid sequence of SEQ ID NO: 1036. In some embodiments, the anti-C3 LCDR1 comprises the amino acid sequence of SEQ ID NO: 967; the anti-C3 LCDR2 comprises the amino acid sequence of SEQ ID NO: 1002; and the anti-C3 LCDR3 comprises the amino acid sequence of SEQ ID NO: 1037. In some embodiments, the anti-C3 LCDR1 comprises the amino acid sequence of SEQ ID NO: 968; the anti-C3 LCDR2 comprises the amino acid sequence of SEQ ID NO: 1003; and the anti-C3 LCDR3 comprises the amino acid sequence of SEQ ID NO: 1038. In some embodiments, the anti-C3 LCDR1 comprises the amino acid sequence of SEQ ID NO: 969; the anti-C3 LCDR2 comprises the amino acid sequence of SEQ ID NO: 1004; and the anti-C3 LCDR3 comprises the amino acid sequence of SEQ ID NO: 1039. In some embodiments, the anti-C3 LCDR1 comprises the amino acid sequence of SEQ ID NO: 970; the anti-C3 LCDR2 comprises the amino acid sequence of SEQ ID NO: 1005; and the anti-C3 LCDR3 comprises the amino acid sequence of SEQ ID NO: 1040. In some embodiments, the anti-C3 LCDR1 comprises the amino acid sequence of SEQ ID NO: 971; the anti-C3 LCDR2 comprises the amino acid sequence of SEQ ID NO: 1006; and the anti-C3 LCDR3 comprises the amino acid sequence of SEQ ID NO: 1041. In some embodiments, the anti-C3 LCDR1 comprises the amino acid sequence of SEQ ID NO: 972; the anti-C3 LCDR2 comprises the amino acid sequence of SEQ ID NO: 1007; and the anti-C3 LCDR3 comprises the amino acid sequence of SEQ ID NO: 1042. In some embodiments, the anti-C3 LCDR1 comprises the amino acid sequence of SEQ ID NO: 973; the anti-C3 LCDR2 comprises the amino acid sequence of SEQ ID NO: 1008; and the anti-C3 LCDR3 comprises the amino acid sequence of SEQ ID NO: 1043. In some embodiments, the anti-C3 LCDR1 comprises the amino acid sequence of SEQ ID NO: 974; the anti-C3 LCDR2 comprises the amino acid sequence of SEQ ID NO: 1009; and the anti-C3 LCDR3 comprises the amino acid sequence of SEQ ID NO: 1044. In some embodiments, the anti-C3 LCDR1 comprises the amino acid sequence of SEQ ID NO: 975; the anti-C3 LCDR2 comprises the amino acid sequence of SEQ ID NO: 1010; and the anti-C3 LCDR3 comprises the amino acid sequence of SEQ ID NO: 1045. In some embodiments, the anti-C3 LCDR1 comprises the amino acid sequence of SEQ ID NO: 976; the anti-C3 LCDR2 comprises the amino acid sequence of SEQ ID NO: 1011; and the anti-C3 LCDR3 comprises the amino acid sequence of SEQ ID NO: 1046. In some embodiments, the anti-C3 LCDR1 comprises the amino acid sequence of SEQ ID NO: 977; the anti-C3 LCDR2 comprises the amino acid sequence of SEQ ID NO: 1012; and the anti-C3 LCDR3 comprises the amino acid sequence of SEQ ID NO: 1047. In some embodiments, the anti-C3 LCDR1 comprises the amino acid sequence of SEQ ID NO: 978; the anti-C3 LCDR2 comprises the amino acid sequence of SEQ ID NO: 1013; and the anti-C3 LCDR3 comprises the amino acid sequence of SEQ ID NO: 1048. In some embodiments, the anti-C3 LCDR1 comprises the amino acid sequence of SEQ ID NO: 979; the anti-C3 LCDR2 comprises the amino acid sequence of SEQ ID NO: 1014; and the anti-C3 LCDR3 comprises the amino acid sequence of SEQ ID NO: 1049. In some embodiments, the anti-C3 LCDR1 comprises the amino acid sequence of SEQ ID NO: 980; the anti-C3 LCDR2 comprises the amino acid sequence of SEQ ID NO: 1015; and the anti-C3 LCDR3 comprises the amino acid sequence of SEQ ID NO: 1050. In some embodiments, the anti-C3 LCDR1 comprises the amino acid sequence of SEQ ID NO: 981; the anti-C3 LCDR2 comprises the amino acid sequence of SEQ ID NO: 1016; and the anti-C3 LCDR3 comprises the amino acid sequence of SEQ ID NO: 1051. In some embodiments, the anti-C3 LCDR1 comprises the amino acid sequence of SEQ ID NO: 982; the anti-C3 LCDR2 comprises the amino acid sequence of SEQ ID NO: 1017; and the anti-C3 LCDR3 comprises the amino acid sequence of SEQ ID NO: 1052.

In some embodiments, the anti-CFHR4 VH comprises an amino acid sequence having at least 90% sequence identity to at least one of SEQ ID NOs: 260-265, 284-293, 324-334, and 368-380. In some embodiments, the anti-CFHR4 VL comprises an amino acid sequence having at least 90% sequence identity to at least one of SEQ ID NOs: 272-277, 304-313, 346-356, and 394-406. In some embodiments, the anti-C3 VH comprises an amino acid sequence having at least 90% (e.g., at least 90%, 91%, 92%, 93%, 94%, 95%, 96%, 97%, 98%, 99%, or 100%) sequence identity to at least one of SEQ ID NOs: 1127-1200. In some embodiments, the anti-C3 VL comprises an amino acid sequence having at least 90% (e.g., at least 90%, 91%, 92%, 93%, 94%, 95%, 96%, 97%, 98%, 99%, or 100%) sequence identity to at least one of SEQ ID NOs: 1275-1348.

In some embodiments, the bispecific antibody binds CFHR4 and/or C3 and reduces complement activation. In some embodiments, the bispecific antibody binds CFHR4b and/or C3b and reduces complement activation. In some embodiments, the bispecific antibody binds human CFHR4 and/or C3 with a K_{D} of about 100 nM or lower.

In some embodiments, the bispecific antibody comprises a half-life extension moiety. In some embodiments, the half-life extension moiety comprises a polypeptide that is at least 20 amino acids in length and comprises any combination of G, A, S T, E, and P residues. In some embodiments, the half-life extension polypeptide is attached to the C-terminus or N-terminus of the antibody.

Embodiments of the present disclosure also include a pharmaceutical composition comprising any of the bispecific antibodies described herein. In some embodiments, the composition is suitable for ocular administration. In some embodiments, ocular administration comprises injection into vitreous fluid. In some embodiments, ocular administration comprises delivering the antibody using a conjunctival insert, a contact lens, a gel, a nanoparticle, a mucoadhesive polymer, an ointment, a solution, a suspension, eye drops, and/or an implant.

Embodiments of the present disclosure also include a method of treating age-related macular degeneration (AMD) comprising administering a pharmaceutical composition comprising an effective amount of any of the bispecific antibodies described herein to a subject in need thereof. In some embodiments, the pharmaceutical composition is administered ocularly and treats at least one AMD symptom. In some embodiments, AMD comprises wet AMD. In some embodiments, AMD comprises dry AMD. In some embodiments, the AMD symptom comprises visual distortion, reduced central vision, blurred vision, and/or difficulty adapting to low light. In some embodiments, administering the pharmaceutical composition reduces complement activation in the subject's eye. In some embodiments, the pharmaceutical composition is administered at a dose ranging from about 0.0001 mg/dose to about 100 mg/dose. In some embodiments, the pharmaceutical composition is administered at a dose ranging from about 0.0001 mg/ml to about 100 mg/ml.

### BRIEF DESCRIPTION OF THE DRAWING(S)

FIG. 1: Representative illustration of the molecular mechanism underlying treatment of advanced Age-related Macular Degeneration (AMD), including Geographic Atrophy (GA) with the anti-CFHR4 antibodies of the present disclosure.

FIG. 2: Representing results (see Table 1) plotting Odds Ratio and confidence intervals of haplotype effect when there is a deletion (tagged by one of two variants) or a pQTL variant on Y402 haplotype. The associations were done in relation to the reference haplotype at OR=1.

FIGS. 3A-3C: Representative ELISA results used to determine antigen positive serum titers of mice immunized with CFHR4, according to three different immunization protocols/cohorts (FIG. 3A—Cohort 1; FIG. 3B—Cohort 2; FIG. 3C—Cohort 3).

FIG. 4: Representative results of the thermal stabilities of the human CFHR4 monoclonal antibodies of the present disclosure (see Table 2, “ATX” antibodies). Thermal stability was assessed via differential scanning fluorimetry (DSF) utilizing the Protein Thermal Shift (PTS) assay.

FIGS. 5A-5B: Representative results of CFHR4 antibody cross-blocking experiments, including data from a representative heatmap analyzing the ability of the antibodies to block one another for binding to the antigen (FIG. 5A), and a representative network plot, which progressively groups antibodies with similar competition profiles.

FIG. 6: Representative results of CFHR4 antibody binding kinetics.

FIG. 7: Representative results of CFHR4 antibody cross-reactivity with Cynomolgus monkey CFHR4b protein (cCFHR4b).

FIG. 8: Representative results of CFHR4 antibody cross-reactivity with human CFHR4a protein.

FIG. 9: Representative results of CFHR4 antibody cross-reactivity with human CFHR3 protein.

FIG. 10: Representative results of CFHR4 antibody cross-reactivity with human CFHL-1 (Y402H) protein.

FIGS. 11A-11D: Representative schematic diagram of a C₃ convertase assembly assay used to evaluate the ability of the CFHR4 antibodies of the present disclosure to affect C3 convertase formation (FIG. 11A). FIG. 11B includes representative dose response curves for the top inhibitory CFHR4 antibodies, and FIG. 11C includes the IC50 values calculated for each antibody tested in FIG. 11B. FIG. 11D provides a representative summary of data demonstrating at least three functional classes of anti-CFHR4 antibodies.

FIGS. 12A-12D: Representative ELISA results used to determine antigen positive serum titers of mice immunized with human C3 and cyno C3, according to different immunization protocols/cohorts (Cohort 1: FIG. 12A (human C3) and FIG. 12B (cyno C3); Cohort 2: FIG. 12C (human C3); FIG. 12D (cyno C3)).

FIG. 13: Representing ELISA results of polyclonal phage pools to confirm enrichment and examine cross reactivity.

FIG. 14: Representative table containing binding kinetics of the C3 antibodies of the present disclosure.

FIG. 15: Representative table containing results of C3 antibody cross-reactivity with human C3b protein, Cynomolgus monkey C3 protein, and human C3a protein.

FIGS. 16A-16B: Representative tables containing binding kinetics of C3 antibodies with respect to human C3 protein (FIG. 16A) and human C3b protein (FIG. 16B).

FIGS. 17A-17D: Representative results of C3 antibody epitope experiments, including a table containing binning allocations (FIG. 17A), data from a representative heatmap analyzing the ability of the antibodies to block one another for binding to the antigen (FIG. 17B), and representative network plots (FIG. 17C and FIG. 17D), which progressively groups antibodies with similar competition profiles.

FIGS. 18A-18E: Representative binning network plots based on recombinant human affinities and cyno binding performed with C3b (FIG. 18A); C3b affinity and ligand blocking (FIG. 18B); bins vs. communities for CFHR4b blocking (FIG. 18C); bins vs. communities for CFH blocking (FIG. 18D); and material availability and common vs. diverse light chains (FIG. 18E).

FIG. 19: Representative results of C3 antibody polyreactivity with baculovirus particles (BVPs).

FIG. 20: Representative results of C3 antibody polyreactivity with the various proteins indicated.

FIGS. 21A-21B: Representative results of C3 antibody polyreactivity with CFHR4 (FIG. 21A) and CFH (FIG. 21B).

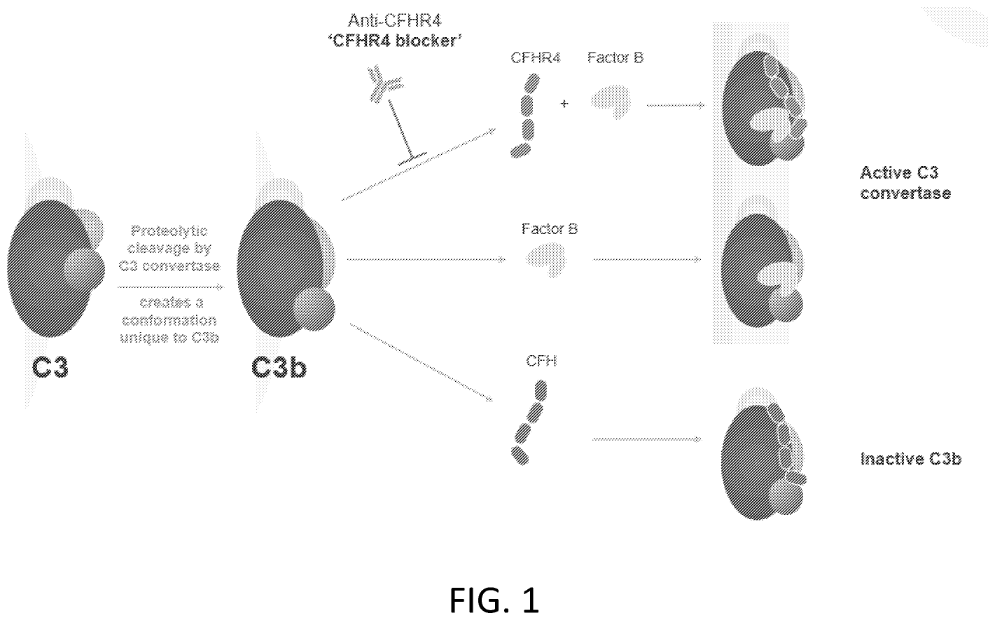

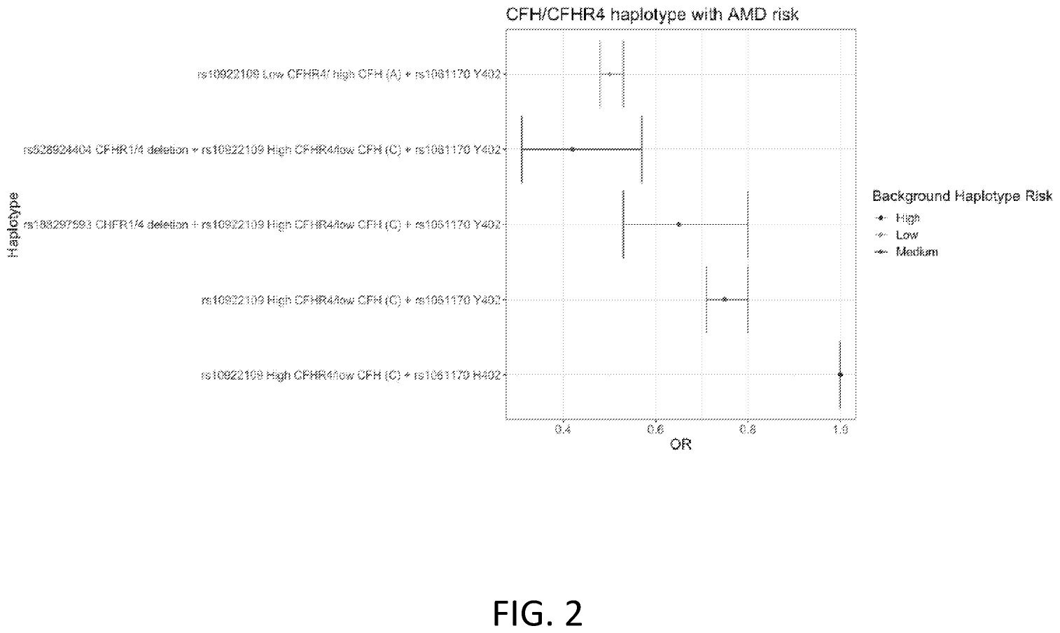

### DETAILED DESCRIPTION

Embodiments of the present disclosure relate to the treatment and/or prevention of age-related macular degeneration (AMD), including both neovascular AMD (“wet” AMD) and non-neovascular AMD (“dry” AMD), as well as advanced forms of non-neovascular AMD (geographic atrophy or GA). In particular, the present disclosure provides novel therapeutic bispecific antibodies that target components of the alternative pathway of the complement activation system.

The human complement system comprises a family of proteins that are essential to the human immune response against infections. Complement recognizes microbes or damaged host cells and subsequently triggers an enzymatic cascade that mainly serves to label target cells for phagocytosis by immune cells, produce chemoattractants, and directly kill target cells via pore formation. Unwanted complement activation on the body's own cells is a key pathological driver in a wide spectrum of immune diseases including autoimmune, inflammatory, and degenerative diseases. For current and future development of therapeutic complement inhibitors, knowledge of complement activation and how it can be regulated is of great importance.

Human complement factor H-related protein (CFHR) 4 belongs to the factor H family of plasma glycoproteins that are composed of short consensus repeat (SCR) domains. Although factor H is a well-known inhibitor of the alternative complement pathway, the functions of the CFHR proteins are not as clear. For example, CFHR4 lacks SCRs homologous to the complement inhibitory domains of factor H; however, its complement regulatory activities are not fully understood. Previous studies have shown that CFHR4 binds C-reactive protein via its most N-terminal SCR, which leads to classical complement pathway activation. CFHR4 binds C3b via its C terminus, but the significance of this interaction is not as well understood. Recent reports indicate that CFHR4 may serve as a platform for the assembly of an alternative pathway C3 convertase by binding C3b. This is at least partially based on the sustained ability of CFHR4-bound C3b to bind factor B and properdin, leading to an active convertase that generates C3a and C3b from C3. The CFHR4-C3bBb convertase is less sensitive to the factor H-mediated decay compared with the C3bBb convertase. CFHR4 mutants containing exchanges of conserved residues within the C-terminal C3b-binding site showed significantly reduced C3b binding and alternative pathway complement activation. These initial data suggest that, in contrast to the complement inhibitor factor H, CFHR4 acts as an enhancer of opsonization by promoting complement activation. CFHR4 is detected in human plasma as two different glycoproteins. The 86-kDa long isoform termed CFHR4A consists of nine SCRs. The ˜45-kDa shorter isoform termed CFHR4B is composed of five SCRs that represent SCR1 and SCR6-9 of CFHR4A. In CFHR4A, SCR1-4 and SCR5-8 are highly related based on nucleotide and amino acid sequence identity, likely as a result of an intramolecular duplication. Like all CFHRs, both CFHR4 isoforms lack SCRs homologous to the N-terminal complement inhibitory domains SCR1-4 of FH and CFHL1. In agreement with this, no significant cofactor and convertase decay accelerating activities have been reported for CFHR4B. The two most C-terminal domains of CFHR4A and CFHR4B are homologous to the C-terminal FH domains SCR19-20 that contain C3b/C3d-binding sites. CFHR4B has been shown to bind the C3d region of C3b via its C-terminal SCR4-5. However, except for a slight enhancement of the cofactor activity of FH in the presence of high CFHR4B concentrations, no significant functions have been associated with this C3b binding capacity. The CFHR4A isoform has not yet been characterized for its interaction with C3b and complement regulatory activity.

Convertase enzymes fulfill a central role in the complement cascade as they cleave C3 and C5, which mediate nearly all complement effector functions. C3 convertases cleave C3 into C3a, a chemoattractant molecule, and C3b, which covalently binds to target surfaces and triggers phagocytosis. C5 convertases cleave C5 into C5a, a potent mediator of leukocyte recruitment and inflammation, and C5b, the initiator of the membrane attack complex and cell lysis. The complement cascade begins via specific recognition of target cells in the classical (CP) and lectin (LP) pathways. In the CP, antibodies bind epitopes on the target cell and subsequently recruit the C1 complex (C1qr₂ₛ₂). Upon binding to the antibody platforms, C1q-associated protease C1s converts C4 and C2 to generate a C3 convertase enzyme (C4b2a) on the cell surface. Similarly, the lectin pathway also forms C4b2a via activation of mannose-binding lectin-associated serine proteases. The resulting CP/LP C4b2a convertases cleave C3 into C3a and C3b. Following cleavage, a reactive thioester in C3b is exposed, which enables its covalent attachment to target cell surfaces, leading to recognition of the cells by phagocytes. The labeling of target cells with C3b is amplified by the alternative pathway (AP) in which surface-bound C3b binds factor B (FB). The proconvertase C3bB is then cleaved by factor D (FD) to form an active C3 convertase complex that consists of C3b and the protease fragment Bb (C3bBb). Since the resulting active AP C3 convertase (C3bBb) is comprised of C3b itself, substrate cleavage results in generation of additional convertases, further propagating C3b deposition. When the density of C3b molecules on the cell surface becomes sufficiently high, the existing C3 convertases (C4b2a and C3bBb) gain the ability to cleave C5, leading to formation of C5a and C5b.

Age-related macular degeneration (AMD) is a progressive retinal disease in which the early stage is characterized by relatively few small drusen within the macula. When AMD progresses, drusen size and number increase, eventually leading towards more advanced stages of AMD. Two forms of advanced AMD are distinguished. The first form, neovascular AMD, is characterized by infiltration of abnormal blood vessels into the retina. These newly formed vessels are fragile and when they break, the leakage of blood constituents in the retina leads to sudden vision loss. The second form of advanced AMD, geographic atrophy (GA), is the result of gradual degeneration of the RPE and photoreceptors cells. Although neovascularization occurs in only 15-20% of AMD cases, it is responsible for the vast majority of vision loss caused by AMD. Drugs targeting vascular endothelial growth factor (VEGF), one of the central molecules in neovascularization, have proven to be very successful in neovascular AMD. However, no treatment is available for the remaining majority of early, intermediate or geographic atrophy AMD cases, and furthermore there are no effective means of preventing progression of early to advanced stages. Although it is known that AMD is the result of a complex interaction of environmental and genetic risk factors, studies into the molecular constituents of drusen suggest that AMD may have an immunological component. This suggestion arose after proteins involved in inflammation and/or other immune-associated responses, including components of the complement system, were found within drusen. Additionally, there are currently no treatments that address both wet and dry forms of AMD in a single biologic molecule.

In light of this, the embodiments of the present disclosure provide a therapeutic platform based on modulating different aspects of the complement activation pathway using bispecific antibodies targeting CFHR4 and C3. As described further herein, the bispecific antibodies of the present disclosure include an antigen binding site directed against CFHR4 and an antigen binding site directed against C3. Such antigen binding sites are described herein, and also in International PCT Appln. No. PCT/US2023/020351, and U.S. Provisional Patent Appln. Serial No. U.S. 63/462,638, both of which are incorporated herein by reference in their entireties and for all purposes.

### Definitions

To facilitate an understanding of the present technology, a number of terms and phrases are defined below. Additional definitions are set forth throughout the detailed description.

The use of the terms “a” and “an” and “the” and “at least one” and similar referents in the context of describing the embodiments of the present disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the various embodiments of the present disclosure, and does not pose a limitation on the scope of these embodiment unless otherwise claimed. No language in the specification should be construed as indicating any non-claimed element as essential to the practice of the various embodiments of the present disclosure.

As used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

The term “one or more,” as used herein, refers to a number higher than one. For example, the term “one or more” encompasses any of the following: two or more, three or more, four or more, five or more, six or more, seven or more, eight or more, nine or more, ten or more, eleven or more, twelve or more, thirteen or more, fourteen or more, fifteen or more, twenty or more, fifty or more, 100 or more, or an even greater number.

The term “one or more but less than a higher number,” “two or more but less than a higher number,” “three or more but less than a higher number,” “four or more but less than a higher number,” “five or more but less than a higher number,” “six or more but less than a higher number,” “seven or more but less than a higher number,” “eight or more but less than a higher number,” “nine or more but less than a higher number,” “ten or more but less than a higher number,” “eleven or more but less than a higher number,” “twelve or more but less than a higher number,” “thirteen or more but less than a higher number,” “fourteen or more but less than a higher number,” or “fifteen or more but less than a higher number” is not limited to a higher number. For example, the higher number can be 10,000, 1,000, 100, 50, etc. For example, the higher number can be approximately 50 (e.g., 50, 49, 48, 47, 46, 45, 44, 43, 42, 41, 40, 39, 38, 37, 36, 35, 34, 33, 32, 31, 32, 30, 29, 28, 27, 26, 25, 24, 23, 22, 21, 20, 19, 18, 17, 16, 15, 14, 13, 12, 11, 10, 9, 8, 7, 6, 5, 4, 3 or 2).

The term “immunoglobulin” or “antibody,” as used herein, refers to a protein that is found in blood or other bodily fluids of vertebrates, which is used by the immune system to identify and neutralize foreign objects, such as bacteria and viruses. Typically, an immunoglobulin or antibody is a protein that comprises at least one complementarity determining region (CDR). The CDRs form the “hypervariable region” of an antibody, which is responsible for antigen binding (discussed further below). A whole antibody typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H1}, C_{H2}, and C_{H3}) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical antibody, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the CDRs. There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)).

The framework regions are connected by three CDRs. As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

As used herein, when an antibody or other entity (e.g., antigen binding domain) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23 (9): 1126-1129 (2005)). Any antigen-binding fragment of the antibody described herein is within the scope of the present disclosure. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and CHI domains, (ii) a F(ab′)2 fragment, which is a bivalent fragment comprising two Fab fragments linked by a disulfide bridge at the hinge region, (iii) a Fv fragment consisting of the VL and VA domains of a single arm of an antibody, (iv) a Fab′ fragment, which results from breaking the disulfide bridge of an F(ab′)2 fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

The term “monoclonal antibody,” as used herein, refers to an antibody produced by a single clone of B lymphocytes that is directed against a single epitope on an antigen. Monoclonal antibodies typically are produced using hybridoma technology, as first described in Köhler and Milstein, Eur. J. Immunol., 5:511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352:624-628 (1991); and Marks et al., J. Mol. Biol., 222:581-597 (1991)), or produced from transgenic mice carrying a fully human immunoglobulin system (see, e.g., Lonberg, Nat. Biotechnol., 23 (9): 1117-25 (2005), and Lonberg, Handb. Exp. Pharmacol., 181:69-97 (2008)). In contrast, “polyclonal” antibodies are antibodies that are secreted by different B cell lineages within an animal. Polyclonal antibodies are a collection of immunoglobulin molecules that recognize multiple epitopes on the same antigen.

The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones.

The terms “nucleic acid,” “polynucleotide,” “nucleotide sequence,” and “oligonucleotide” are used interchangeably herein and refer to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands affects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a “gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located S′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and polyadenylation.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “wild-type” when made in reference to a protein refers to a protein that has the characteristics of a naturally occurring protein. The term “naturally-occurring” as applied to an object refers to the fact that an object can be found in nature. For example, a polypeptide or polynucleotide sequence that is present in an organism (including viruses) that can be isolated from a source in nature, and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population, or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

Nucleic acid or amino acid sequence “identity,” as described herein, can be determined by comparing a nucleic acid or amino acid sequence of interest to a reference nucleic acid or amino acid sequence. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215 (3): 403-410 (1990), Beigert et al., Proc. Natl. Acad. Sci. USA, 106 (10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21 (7): 951-960 (2005), Altschul et al., Nucleic Acids Res., 25 (17): 3389-3402 (1997), and Gusfield, Algorithms on Strings, Trees and Sequences, Cambridge University Press, Cambridge UK (1997)).

As would be recognized by one of ordinary skill in the art based on the present disclosure, one or more amino acids of the aforementioned bispecific antibodies, or antigen fragments thereof, can be replaced or substituted with a different amino acid. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg). Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine. Aromatic amino acids may be sub-divided into two sub-groups: the “nitrogen ring sub-group” consisting of histidine and tryptophan and the “phenyl sub-group” consisting of phenylalanine and tyrosine.

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure. Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In addition, one or more amino acids can be inserted into the bispecific antibodies of the present disclosure (e.g., insertion into the heavy and/or light chain variable region amino acid sequence). Any number of suitable amino acids can be inserted into the amino acid sequence of the antibody or antigen-binding fragment thereof. In this respect, at least one amino acid (e.g., 2 or more, 5 or more, or 10 or more amino acids), but not more than 20 amino acids (e.g., 18 or less, 15 or less, or 12 or less amino acids), can be inserted into the amino acid sequence of the antibody or antigen-binding fragment thereof. For example, 1-10 amino acids (e.g., 1, 2, 3, 4, 5, 6, 7, 8, 9, or 10 amino acids) may be inserted into the amino acid sequence of the monoclonal antibody or antigen-binding fragment thereof. In this respect, the amino acid(s) can be inserted into an antibody or antigen-binding fragment thereof in any suitable location. Preferably, the amino acid(s) are inserted into a CDR (e.g., CDR1, CDR2, or CDR3) of the antibody or antigen-binding fragment thereof.

The terms “immunogen” and “antigen” are used interchangeably herein and refer to any molecule, compound, or substance that induces an immune response in an animal (e.g., a mammal). An “immune response” can entail, for example, antibody production and/or the activation of immune effector cells. An antigen in the context of the disclosure can comprise any subunit, fragment, or epitope of any proteinaceous or non-proteinaceous (e.g., carbohydrate or lipid) molecule that provokes an immune response in a mammal. The term “epitope” refers to a sequence of an antigen that is recognized by an antibody or an antigen receptor. Epitopes also are referred to in the art as “antigenic determinants.” In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. The antigen can be a protein or peptide of viral, bacterial, parasitic, fungal, protozoan, prion, cellular, or extracellular origin, which provokes an immune response in a mammal, preferably leading to protective immunity.

A “pharmaceutically acceptable carrier” as used herein generally refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “pharmaceutical formulation” as used herein generally refers to a preparation which is in such form as to permit the biological activity of an active ingredient (e.g., bispecific antibodies of the present disclosure) contained therein to be effective, and which contains no additional components which are unacceptably toxic to a subject to which the formulation would be administered.

As used herein, “treatment” (and grammatical variations thereof such as “treat” or “treating”) generally refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, bispecific antibodies of the present disclosure or other compositions that include a bispecific antibody of the present disclosure (e.g., an antibody conjugate, a fusion protein, or a polymeric formulation) are used to delay development of a disease or to slow the progression of a disease.

The term “half-life” as used herein generally refers to the time required for the concentration of a substance (e.g., a bispecific antibody, an antibody conjugate, a fusion protein (e.g., a Fab fusion protein), or a polymeric formulation) to decrease by one-half, in vivo (e.g., in the eye (e.g., the vitreous)) or in vitro.

An “effective amount” of an agent, e.g., a pharmaceutical formulation, as used herein generally refers to an amount effective, at dosages and for periods of time necessary, to achieve the desired therapeutic or prophylactic result.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. A “subject” may be a “patient.”

### Anti-CFHR4/Anti-C3 Bispecific Antibodies

As described further herein, embodiments of the present disclosure relate to the treatment and/or prevention of age-related macular degeneration (AMD), including both neovascular AMD (“wet” AMD) and non-neovascular AMD (“dry” AMD), as well as advanced forms of non-neovascular AMD (geographic atrophy or GA). In particular, the present disclosure provides novel therapeutic bispecific antibodies that target components of the alternative pathway of the complement activation system to treat AMD. As described further herein, anti-CFHR4 antibodies and anti-C3 were generated, and their structural and functional properties were elucidated. Based on these data, corresponding bispecific antibodies were generated in which one antigen-binding site is directed against CFHR4 and the other antigen-binding site is directed against C3.

In accordance with these embodiments, the present disclosure provides a bispecific antibody, or an antigen binding fragment thereof, which specifically binds human Complement Factor H Related 4 (CFHR) and/or human Complement Component 3 (C3). In some embodiments, the human CFHR4 is the CFHR4 b variant (CFHR4b), which is optionally a polypeptide comprising or consisting of the amino acid sequence of SEQ ID NO: 580. As such, the bispecific antibodies of the present disclosure are capable of binding CFHR4 and/or CFHR4B polypeptides and proteins. In some embodiments, the human C3 is the b variant of C3 (C3b), which is optionally a polypeptide comprising or consisting of the amino acid sequence of SEQ ID NO: 1645. As such, the bispecific antibodies of the present disclosure are capable of binding Complement Component 3 (C3), Complement Component 3a (C3a), and/or Complement Component 3b (C3b) polypeptides and proteins.

In some embodiments, the bispecific antibody or fragment thereof is monoclonal, optionally recombinant. In some embodiments, the bispecific antibody or fragment thereof is human, humanized, or chimeric. In some embodiments, the bispecific antibody or fragment thereof is a full length antibody, a single chain antibody, a single chain variable fragment (scFv), a variable fragment (Fv), a fragment antigen-binding region (Fab), a Fab-C, a Fab′-SH, a (Fab′)2, a single-domain antibody (sdAb), a VHH antibody, a nanobody, a camelid-derived single-domain antibody, a shark IgNAR-derived single-domain antibody fragment (VNAR), a diabody, a triabody, an anticalin or an aptamer, optionally wherein the antibody is a full length antibody comprising an Fc region such as a human IgG1, IgG2, IgG3 or IgG4 region.

In some embodiments, the bispecific antibody or fragment thereof is conjugated to at least one additional moiety, optionally selected from: an antigen binding moiety, such as an antibody or antigen-binding fragment thereof, which is capable of specific binding to a target which is not human CFHR4 or human C3, preferably wherein said target is expressed in the human eye; a therapeutic or cytotoxic moiety; a detection moiety; a purification moiety; a half-life extension moiety, optionally a polypeptide that is at least 20 amino acids in length and comprises any combination of G, A, S T, E, and P residue, which polypeptide is conjugated to the C- or N-terminus of the antibody.

In some embodiments, the bispecific antibody or fragment thereof is a polypeptide comprising: one, two or all three HCDRs of any one of the exemplary CFHR4 antibodies the sequences of which are provided in Table 3; and one, two or all three of the corresponding LCDRs of said exemplary antibody; and/or a VH sequence having at least 90% identity to the VH sequence of any one of the exemplary CFHR4 antibodies the sequences of which are provided in Table 4; and optionally a VL sequence having at least 90% identity to the corresponding VL sequence of said exemplary antibody, preferably wherein variation is not permitted in the HCDRs or LCDRs; and/or all six CDRs of any one of the exemplary CFHR4 antibodies the sequences of which are provided in Table 3; the VH and VL sequences of any one of the exemplary CFHR4 antibodies the sequences of which are provided in Table 4; and/or the full length heavy chain (VH+constant) sequence of any one of the exemplary CFHR4 antibodies the sequences of which are provided in Table 5; and optionally the corresponding full length light chain (VL+constant) sequence of said exemplary antibody.

In some embodiments, the bispecific antibody or fragment thereof is a polypeptide comprising: one, two or all three HCDRs of any one of the exemplary C3 antibodies the sequences of which are provided in Table 6; and one, two or all three of the corresponding LCDRs of said exemplary antibody; and/or a VH sequence having at least 90% identity to the VH sequence of any one of the exemplary C3 antibodies the sequences of which are provided in Table 7; and optionally a VL sequence having at least 90% identity to the corresponding VL sequence of said exemplary antibody provided in Table 8, preferably wherein variation is not permitted in the HCDRs or LCDRs; and/or all six CDRs of any one of the exemplary C3 antibodies the sequences of which are provided in Table 6; the VH and VL sequences of any one of the exemplary C3 antibodies the sequences of which are provided in Tables 7 and 8; and/or the full length heavy chain (VH+constant) sequence of any one of the exemplary C3 antibodies the sequences of which are provided in Table 9; and optionally the corresponding full length light chain (VL+constant) sequence of said exemplary antibody provided in Table 10.

Embodiments of the present disclosure also include a polynucleotide encoding the bispecific antibody or fragment thereof. In some embodiments, the polynucleotide comprises or consists of a nucleic acid sequence having at least 70%, 80%, 90%, or 100% identity to a nucleic acid sequence of any one of the exemplary antibodies the sequences of which are provided in Tables 5, 9, and 10. Embodiments of the present disclosure also include an expression vector comprising the polynucleotide, which is optionally an adeno-associated virus (AAV) vector, a lentiviral (LV) vector, a herpes simplex virus (HSV) vector, or a retrovirus vector.

Embodiments of the present disclosure also include a pharmaceutical composition comprising the bispecific antibody or fragment thereof, the corresponding polynucleotide, or the vector comprising the polynucleotide. In some embodiments, the composition comprises at least one pharmaceutically acceptable carrier, diluent or preservative; and/or at least one additional active ingredient. In some embodiments, the pharmaceutical composition is suitable for ocular administration to a subject, optionally by delivery using a conjunctival insert, a contact lens, a gel, a nanoparticle, a mucoadhesive polymer, an ointment, a solution, a suspension, eye drops, and/or an implant, preferably by injection into the vitreous fluid.

Embodiments of the present disclosure also include the bispecific antibody or fragment thereof, the corresponding polynucleotide, the vector comprising the polynucleotide, or the pharmaceutical composition, for use as a medicament, optionally for use in a method of treating a disease of the eye in a subject. In some embodiments, the disease is characterized by increased activation of the complement system, in particular the alternative pathway, and in particular within the eye of the subject, for example in drusen or retinal pigment epithelium (RPE) cells of the subject. In some embodiments, the method comprises ocular administration of the antibody, preferably by injection into the vitreous fluid, and said administration preferably relieves at least one symptom in the subject selected from visual distortion, reduced central vision, blurred vision, and/or difficulty adapting to low light. In some embodiments, the disease is age-related macular degeneration (AMD), including dry AMD, which may be at an early, intermediate or advanced stage (the latter otherwise known as geographic atrophy, GA).

In accordance with the above, embodiments of the present disclosure include a bispecific antibody comprising a first antigen-binding site directed against Complement Factor H Related 4 (CFHR4) and a second antigen-binding site directed against Complement Component 3 (C3). In some embodiments, the first anti-CFHR4 antigen-binding site comprises a heavy chain variable region (VH) comprising complementarity determining regions (CDRs) HCDR1, HCDR2, and HCDR3 from at least one of SEQ ID NOs: 260-265, 284-293, 324-334, and 368-380; and a light chain variable region (VL) comprising complementarity determining regions (CDRs) LCDR1, LCDR2, and LCDR3 from at least one of SEQ ID NOs: 272-277, 304-313, 346-356, and 394-406; and the second anti-C3 antigen-binding site comprises a heavy chain variable region (VH) comprising complementarity determining regions (CDRs) HCDR1, HCDR2, and HCDR3; and a light chain variable region (VL) comprising complementarity determining regions (CDRs) LCDR1, LCDR2, and LCDR3.

Embodiments of the present disclosure include a bispecific antibody comprising a first antigen-binding site directed against Complement Factor H Related 4 (CFHR4) and a second antigen-binding site directed against Complement Component 3 (C3). In some embodiments, the first anti-CFHR4 antigen-binding site comprises a heavy chain variable region (VH) comprising complementarity determining regions (CDRs) HCDR1, HCDR2, and HCDR3; and a light chain variable region (VL) comprising complementarity determining regions (CDRs) LCDR1, LCDR2, and LCDR3; and the second anti-C3 antigen-binding site comprises a heavy chain variable region (VH) comprising complementarity determining regions (CDRs) HCDR1, HCDR2, and HCDR3 from at least one of SEQ ID NOs: 1127-1200; and a light chain variable region (VL) comprising complementarity determining regions (CDRs) LCDR1, LCDR2, and LCDR3 from at least one of SEQ ID NOs: 1275-1348.

Embodiments of the present disclosure include a bispecific antibody comprising a first antigen-binding site directed against Complement Factor H Related 4 (CFHR4) and a second antigen-binding site directed against Complement Component 3 (C3). In some embodiments, the first anti-CFHR4 antigen-binding site comprises a heavy chain variable region (VH) comprising complementarity determining regions (CDRs) HCDR1, HCDR2, and HCDR3 from at least one of SEQ ID NOs: 260-265, 284-293, 324-334, and 368-380; and a light chain variable region (VL) comprising complementarity determining regions (CDRs) LCDR1, LCDR2, and LCDR3 from at least one of SEQ ID NOs: 272-277, 304-313, 346-356, and 394-406; and the second anti-C3 antigen-binding site comprises a heavy chain variable region (VH) comprising complementarity determining regions (CDRs) HCDR1, HCDR2, and HCDR3 from at least one of SEQ ID NOs: 1127-1200; and a light chain variable region (VL) comprising complementarity determining regions (CDRs) LCDR1, LCDR2, and LCDR3 from at least one of SEQ ID NOs: 1275-1348.

In some embodiments, the bispecific antibodies of the present disclosure comprise an anti-CFHR4 VH that comprises an amino acid sequence having at least 90% sequence identity to at least one of SEQ ID NOs: 260-265, 284-293, 324-334, and 368-380. In some embodiments, the bispecific antibodies of the present disclosure comprise an anti-CFHR4 VL that comprises an amino acid sequence having at least 90% sequence identity to at least one of SEQ ID NOs: 272-277, 304-313, 346-356, and 394-406.

In accordance with these embodiments, the anti-CFHR4 HCDR1 comprises the amino acid sequence of SEQ ID NO: 2; the anti-CFHR4 HCDR2 comprises the amino acid sequence of SEQ ID NO: 9; and the anti-CFHR4 HCDR3 comprises the amino acid sequence of SEQ ID NO: 15. In some embodiments, the anti-CFHR4 HCDR1 comprises the amino acid sequence of SEQ ID NO: 3; the anti-CFHR4 HCDR2 comprises the amino acid sequence of SEQ ID NO: 10; and the anti-CFHR4 HCDR3 comprises the amino acid sequence of SEQ ID NO: 16. In some embodiments, the anti-CFHR4 HCDR1 comprises the amino acid sequence of SEQ ID NO: 4; the anti-CFHR4 HCDR2 comprises the amino acid sequence of SEQ ID NO: 11; and the anti-CFHR4 HCDR3 comprises the amino acid sequence of SEQ ID NO: 17. In some embodiments, the anti-CFHR4 HCDR1 comprises the amino acid sequence of SEQ ID NO: 5; the anti-CFHR4 HCDR2 comprises the amino acid sequence of SEQ ID NO: 12; and the anti-CFHR4 HCDR3 comprises the amino acid sequence of SEQ ID NO: 18. In some embodiments, the anti-CFHR4 HCDR1 comprises the amino acid sequence of SEQ ID NO: 6; the anti-CFHR4 HCDR2 comprises the amino acid sequence of SEQ ID NO: 13; and the anti-CFHR4 HCDR3 comprises the amino acid sequence of SEQ ID NO: 19. In some embodiments, the anti-CFHR4 HCDR1 comprises the amino acid sequence of SEQ ID NO: 7; the anti-CFHR4 HCDR2 comprises the amino acid sequence of SEQ ID NO: 14; and the anti-CFHR4 HCDR3 comprises the amino acid sequence of SEQ ID NO: 20. In some embodiments, the anti-CFHR4 HCDR1 comprises the amino acid sequence of SEQ ID NO: 22; the anti-CFHR4 HCDR2 comprises the amino acid sequence of SEQ ID NO: 33; and the anti-CFHR4 HCDR3 comprises the amino acid sequence of SEQ ID NO: 43. In some embodiments, the anti-CFHR4 HCDR1 comprises the amino acid sequence of SEQ ID NO: 23; the anti-CFHR4 HCDR2 comprises the amino acid sequence of SEQ ID NO: 34; and the anti-CFHR4 HCDR3 comprises the amino acid sequence of SEQ ID NO: 44. In some embodiments, the anti-CFHR4 HCDR1 comprises the amino acid sequence of SEQ ID NO: 24; the anti-CFHR4 HCDR2 comprises the amino acid sequence of SEQ ID NO: 35; and the anti-CFHR4 HCDR3 comprises the amino acid sequence of SEQ ID NO: 45. In some embodiments, the anti-CFHR4 HCDR1 comprises the amino acid sequence of SEQ ID NO: 25; the anti-CFHR4 HCDR2 comprises the amino acid sequence of SEQ ID NO: 36; and the anti-CFHR4 HCDR3 comprises the amino acid sequence of SEQ ID NO: 46. In some embodiments, the anti-CFHR4 HCDR1 comprises the amino acid sequence of SEQ ID NO: 26; the anti-CFHR4 HCDR2 comprises the amino acid sequence of SEQ ID NO: 37; and the anti-CFHR4 HCDR3 comprises the amino acid sequence of SEQ ID NO: 47. In some embodiments, the anti-CFHR4 HCDR1 comprises the amino acid sequence of SEQ ID NO: 27; the anti-CFHR4 HCDR2 comprises the amino acid sequence of SEQ ID NO: 38; and the anti-CFHR4 HCDR3 comprises the amino acid sequence of SEQ ID NO: 48. In some embodiments, the anti-CFHR4 HCDR1 comprises the amino acid sequence of SEQ ID NO: 28; the anti-CFHR4 HCDR2 comprises the amino acid sequence of SEQ ID NO: 39; and the anti-CFHR4 HCDR3 comprises the amino acid sequence of SEQ ID NO: 49. In some embodiments, the anti-CFHR4 HCDR1 comprises the amino acid sequence of SEQ ID NO: 29; the anti-CFHR4 HCDR2 comprises the amino acid sequence of SEQ ID NO: 40; and the anti-CFHR4 HCDR3 comprises the amino acid sequence of SEQ ID NO: 50. In some embodiments, the anti-CFHR4 HCDR1 comprises the amino acid sequence of SEQ ID NO: 30; the anti-CFHR4 HCDR2 comprises the amino acid sequence of SEQ ID NO: 41; and the anti-CFHR4 HCDR3 comprises the amino acid sequence of SEQ ID NO: 51. In some embodiments, the anti-CFHR4 HCDR1 comprises the amino acid sequence of SEQ ID NO: 31; the anti-CFHR4 HCDR2 comprises the amino acid sequence of SEQ ID NO: 42; and the anti-CFHR4 HCDR3 comprises the amino acid sequence of SEQ ID NO: 52. In some embodiments, the anti-CFHR4 HCDR1 comprises the amino acid sequence of SEQ ID NO: 54; the anti-CFHR4 HCDR2 comprises the amino acid sequence of SEQ ID NO: 66; and the anti-CFHR4 HCDR3 comprises the amino acid sequence of SEQ ID NO: 77. In some embodiments, the anti-CFHR4 HCDR1 comprises the amino acid sequence of SEQ ID NO: 55; the anti-CFHR4 HCDR2 comprises the amino acid sequence of SEQ ID NO: 67; and the anti-CFHR4 HCDR3 comprises the amino acid sequence of SEQ ID NO: 78. In some embodiments, the anti-CFHR4 HCDR1 comprises the amino acid sequence of SEQ ID NO: 56; the anti-CFHR4 HCDR2 comprises the amino acid sequence of SEQ ID NO: 68; and the anti-CFHR4 HCDR3 comprises the amino acid sequence of SEQ ID NO: 79. In some embodiments, the anti-CFHR4 HCDR1 comprises the amino acid sequence of SEQ ID NO: 57; the anti-CFHR4 HCDR2 comprises the amino acid sequence of SEQ ID NO: 69; and the anti-CFHR4 HCDR3 comprises the amino acid sequence of SEQ ID NO: 80. In some embodiments, the anti-CFHR4 HCDR1 comprises the amino acid sequence of SEQ ID NO: 58; the anti-CFHR4 HCDR2 comprises the amino acid sequence of SEQ ID NO: 70; and the anti-CFHR4 HCDR3 comprises the amino acid sequence of SEQ ID NO: 81. In some embodiments, the anti-CFHR4 HCDR1 comprises the amino acid sequence of SEQ ID NO: 59; the anti-CFHR4 HCDR2 comprises the amino acid sequence of SEQ ID NO: 71; and the anti-CFHR4 HCDR3 comprises the amino acid sequence of SEQ ID NO: 82. In some embodiments, the anti-CFHR4 HCDR1 comprises the amino acid sequence of SEQ ID NO: 60; the anti-CFHR4 HCDR2 comprises the amino acid sequence of SEQ ID NO: 72; and the anti-CFHR4 HCDR3 comprises the amino acid sequence of SEQ ID NO: 83. In some embodiments, the anti-CFHR4 HCDR1 comprises the amino acid sequence of SEQ ID NO: 61; the anti-CFHR4 HCDR2 comprises the amino acid sequence of SEQ ID NO: 73; and the anti-CFHR4 HCDR3 comprises the amino acid sequence of SEQ ID NO: 84. In some embodiments, the anti-CFHR4 HCDR1 comprises the amino acid sequence of SEQ ID NO: 62; the anti-CFHR4 HCDR2 comprises the amino acid sequence of SEQ ID NO: 74; and the anti-CFHR4 HCDR3 comprises the amino acid sequence of SEQ ID NO: 85. In some embodiments, the anti-CFHR4 HCDR1 comprises the amino acid sequence of SEQ ID NO: 63; the anti-CFHR4 HCDR2 comprises the amino acid sequence of SEQ ID NO: 75; and the anti-CFHR4 HCDR3 comprises the amino acid sequence of SEQ ID NO: 86. In some embodiments, the anti-CFHR4 HCDR1 comprises the amino acid sequence of SEQ ID NO: 64; the anti-CFHR4 HCDR2 comprises the amino acid sequence of SEQ ID NO: 76; and the anti-CFHR4 HCDR3 comprises the amino acid sequence of SEQ ID NO: 87. In some embodiments, the anti-CFHR4 HCDR1 comprises the amino acid sequence of SEQ ID NO: 89; the anti-CFHR4 HCDR2 comprises the amino acid sequence of SEQ ID NO: 103; and the anti-CFHR4 HCDR3 comprises the amino acid sequence of SEQ ID NO: 116. In some embodiments, the anti-CFHR4 HCDR1 comprises the amino acid sequence of SEQ ID NO: 90; the anti-CFHR4 HCDR2 comprises the amino acid sequence of SEQ ID NO: 104; and the anti-CFHR4 HCDR3 comprises the amino acid sequence of SEQ ID NO: 117. In some embodiments, the anti-CFHR4 HCDR1 comprises the amino acid sequence of SEQ ID NO: 91; the anti-CFHR4 HCDR2 comprises the amino acid sequence of SEQ ID NO: 105; and the anti-CFHR4 HCDR3 comprises the amino acid sequence of SEQ ID NO: 118. In some embodiments, the anti-CFHR4 HCDR1 comprises the amino acid sequence of SEQ ID NO: 92; the anti-CFHR4 HCDR2 comprises the amino acid sequence of SEQ ID NO: 106; and the anti-CFHR4 HCDR3 comprises the amino acid sequence of SEQ ID NO: 119. In some embodiments, the anti-CFHR4 HCDR1 comprises the amino acid sequence of SEQ ID NO: 93; the anti-CFHR4 HCDR2 comprises the amino acid sequence of SEQ ID NO: 107; and the anti-CFHR4 HCDR3 comprises the amino acid sequence of SEQ ID NO: 120. In some embodiments, the anti-CFHR4 HCDR1 comprises the amino acid sequence of SEQ ID NO: 94; the anti-CFHR4 HCDR2 comprises the amino acid sequence of SEQ ID NO: 108; and the anti-CFHR4 HCDR3 comprises the amino acid sequence of SEQ ID NO: 121. In some embodiments, the anti-CFHR4 HCDR1 comprises the amino acid sequence of SEQ ID NO: 95; the anti-CFHR4 HCDR2 comprises the amino acid sequence of SEQ ID NO: 109; and the anti-CFHR4 HCDR3 comprises the amino acid sequence of SEQ ID NO: 122. In some embodiments, the anti-CFHR4 HCDR1 comprises the amino acid sequence of SEQ ID NO: 96; the anti-CFHR4 HCDR2 comprises the amino acid sequence of SEQ ID NO: 110; and the anti-CFHR4 HCDR3 comprises the amino acid sequence of SEQ ID NO: 123. In some embodiments, the anti-CFHR4 HCDR1 comprises the amino acid sequence of SEQ ID NO: 97; the anti-CFHR4 HCDR2 comprises the amino acid sequence of SEQ ID NO: 111; and the anti-CFHR4 HCDR3 comprises the amino acid sequence of SEQ ID NO: 124. In some embodiments, the anti-CFHR4 HCDR1 comprises the amino acid sequence of SEQ ID NO: 98; the anti-CFHR4 HCDR2 comprises the amino acid sequence of SEQ ID NO: 112; and the anti-CFHR4 HCDR3 comprises the amino acid sequence of SEQ ID NO: 125. In some embodiments, the anti-CFHR4 HCDR1 comprises the amino acid sequence of SEQ ID NO: 99; the anti-CFHR4 HCDR2 comprises the amino acid sequence of SEQ ID NO: 113; and the HCDR3 comprises the amino acid sequence of SEQ ID NO: 126. In some embodiments, the anti-CFHR4 HCDR1 comprises the amino acid sequence of SEQ ID NO: 100; the HCDR2 comprises the amino acid sequence of SEQ ID NO: 114; and the HCDR3 comprises the amino acid sequence of SEQ ID NO: 127. In some embodiments, the anti-CFHR4 HCDR1 comprises the amino acid sequence of SEQ ID NO: 101; the anti-CFHR4 HCDR2 comprises the amino acid sequence of SEQ ID NO: 115; and the anti-CFHR4 HCDR3 comprises the amino acid sequence of SEQ ID NO: 128.

In some embodiments, the anti-CFHR4 LCDR1 comprises the amino acid sequence of SEQ ID NO: 130; the anti-CFHR4 LCDR2 comprises the amino acid sequence of SEQ ID NO: 153; and the anti-CFHR4 LCDR3 comprises the amino acid sequence of SEQ ID NO: 175. In some embodiments, the anti-CFHR4 LCDR1 comprises the amino acid sequence of SEQ ID NO: 131; the anti-CFHR4 LCDR2 comprises the amino acid sequence of SEQ ID NO: 154; and the anti-CFHR4 LCDR3 comprises the amino acid sequence of SEQ ID NO: 176. In some embodiments, the anti-CFHR4 LCDR1 comprises the amino acid sequence of SEQ ID NO: 132; the anti-CFHR4 LCDR2 comprises the amino acid sequence of SEQ ID NO: 155; and the anti-CFHR4 LCDR3 comprises the amino acid sequence of SEQ ID NO: 177. In some embodiments, the anti-CFHR4 LCDR1 comprises the amino acid sequence of SEQ ID NO: 133; the anti-CFHR4 LCDR2 comprises the amino acid sequence of SEQ ID NO: 156; and the anti-CFHR4 LCDR3 comprises the amino acid sequence of SEQ ID NO: 178. In some embodiments, the anti-CFHR4 LCDR1 comprises the amino acid sequence of SEQ ID NO: 134; the anti-CFHR4 LCDR2 comprises the amino acid sequence of SEQ ID NO: 157; and the anti-CFHR4 LCDR3 comprises the amino acid sequence of SEQ ID NO: 179. In some embodiments, the anti-CFHR4 LCDR1 comprises the amino acid sequence of SEQ ID NO: 135; the anti-CFHR4 LCDR2 comprises the amino acid sequence of SEQ ID NO: 158; and the anti-CFHR4 LCDR3 comprises the amino acid sequence of SEQ ID NO: 180. In some embodiments, the anti-CFHR4 LCDR1 comprises the amino acid sequence of SEQ ID NO: 136; the anti-CFHR4 LCDR2 comprises the amino acid sequence of SEQ ID NO: 159; and the anti-CFHR4 LCDR3 comprises the amino acid sequence of SEQ ID NO: 181. In some embodiments, the anti-CFHR4 LCDR1 comprises the amino acid sequence of SEQ ID NO: 137; the anti-CFHR4 LCDR2 comprises the amino acid sequence of SEQ ID NO: 160; and the anti-CFHR4 LCDR3 comprises the amino acid sequence of SEQ ID NO: 182. In some embodiments, the anti-CFHR4 LCDR1 comprises the amino acid sequence of SEQ ID NO: 138; the anti-CFHR4 LCDR2 comprises the amino acid sequence of SEQ ID NO: 161; and the anti-CFHR4 LCDR3 comprises the amino acid sequence of SEQ ID NO: 183. In some embodiments, the anti-CFHR4 LCDR1 comprises the amino acid sequence of SEQ ID NO: 139; the anti-CFHR4 LCDR2 comprises the amino acid sequence of SEQ ID NO: 162; and the anti-CFHR4 LCDR3 comprises the amino acid sequence of SEQ ID NO: 184. In some embodiments, the anti-CFHR4 LCDR1 comprises the amino acid sequence of SEQ ID NO: 140; the anti-CFHR4 LCDR2 comprises the amino acid sequence of SEQ ID NO: 163; and the anti-CFHR4 LCDR3 comprises the amino acid sequence of SEQ ID NO: 185. In some embodiments, the anti-CFHR4 LCDR1 comprises the amino acid sequence of SEQ ID NO: 141; the anti-CFHR4 LCDR2 comprises the amino acid sequence of SEQ ID NO: 164; and the anti-CFHR4 LCDR3 comprises the amino acid sequence of SEQ ID NO: 186. In some embodiments, the anti-CFHR4 LCDR1 comprises the amino acid sequence of SEQ ID NO: 142; the anti-CFHR4 LCDR2 comprises the amino acid sequence of SEQ ID NO: 165; and the anti-CFHR4 LCDR3 comprises the amino acid sequence of SEQ ID NO: 187. In some embodiments, the anti-CFHR4 LCDR1 comprises the amino acid sequence of SEQ ID NO: 143; the anti-CFHR4 LCDR2 comprises the amino acid sequence of SEQ ID NO: 166; and the anti-CFHR4 LCDR3 comprises the amino acid sequence of SEQ ID NO: 188. In some embodiments, the anti-CFHR4 LCDR1 comprises the amino acid sequence of SEQ ID NO: 144; the anti-CFHR4 LCDR2 comprises the amino acid sequence of SEQ ID NO: 167; and the anti-CFHR4 LCDR3 comprises the amino acid sequence of SEQ ID NO: 189. In some embodiments, the anti-CFHR4 LCDR1 comprises the amino acid sequence of SEQ ID NO: 145; the anti-CFHR4 LCDR2 comprises the amino acid sequence of SEQ ID NO: 168; and the anti-CFHR4 LCDR3 comprises the amino acid sequence of SEQ ID NO: 190. In some embodiments, the anti-CFHR4 LCDR1 comprises the amino acid sequence of SEQ ID NO: 146; the anti-CFHR4 LCDR2 comprises the amino acid sequence of SEQ ID NO: 169; and the anti-CFHR4 LCDR3 comprises the amino acid sequence of SEQ ID NO: 191. In some embodiments, the anti-CFHR4 LCDR1 comprises the amino acid sequence of SEQ ID NO: 147; the anti-CFHR4 LCDR2 comprises the amino acid sequence of SEQ ID NO: 170; and the anti-CFHR4 LCDR3 comprises the amino acid sequence of SEQ ID NO: 192. In some embodiments, the anti-CFHR4 LCDR1 comprises the amino acid sequence of SEQ ID NO: 148; the anti-CFHR4 LCDR2 comprises the amino acid sequence of SEQ ID NO: 171; and the anti-CFHR4 LCDR3 comprises the amino acid sequence of SEQ ID NO: 193. In some embodiments, the anti-CFHR4 LCDR1 comprises the amino acid sequence of SEQ ID NO: 149; the anti-CFHR4 LCDR2 comprises the amino acid sequence of SEQ ID NO: 172; and the anti-CFHR4 LCDR3 comprises the amino acid sequence of SEQ ID NO: 194. In some embodiments, the anti-CFHR4 LCDR1 comprises the amino acid sequence of SEQ ID NO: 150; the anti-CFHR4 LCDR2 comprises the amino acid sequence of SEQ ID NO: 173; and the anti-CFHR4 LCDR3 comprises the amino acid sequence of SEQ ID NO: 195. In some embodiments, the anti-CFHR4 LCDR1 comprises the amino acid sequence of SEQ ID NO: 151; the anti-CFHR4 LCDR2 comprises the amino acid sequence of SEQ ID NO: 174; and the anti-CFHR4 LCDR3 comprises the amino acid sequence of SEQ ID NO: 196. In some embodiments, the anti-CFHR4 LCDR1 comprises the amino acid sequence of SEQ ID NO: 198; the anti-CFHR4 LCDR2 comprises the amino acid sequence of SEQ ID NO: 201; and the anti-CFHR4 LCDR3 comprises the amino acid sequence of SEQ ID NO: 204. In some embodiments, the anti-CFHR4 LCDR1 comprises the amino acid sequence of SEQ ID NO: 199; the anti-CFHR4 LCDR2 comprises the amino acid sequence of SEQ ID NO: 202; and the anti-CFHR4 LCDR3 comprises the amino acid sequence of SEQ ID NO: 205. In some embodiments, the anti-CFHR4 LCDR1 comprises the amino acid sequence of SEQ ID NO: 207; the anti-CFHR4 LCDR2 comprises the amino acid sequence of SEQ ID NO: 217; and the anti-CFHR4 LCDR3 comprises the amino acid sequence of SEQ ID NO: 227. In some embodiments, the anti-CFHR4 LCDR1 comprises the amino acid sequence of SEQ ID NO: 208; the anti-CFHR4 LCDR2 comprises the amino acid sequence of SEQ ID NO: 218; and the anti-CFHR4 LCDR3 comprises the amino acid sequence of SEQ ID NO: 228. In some embodiments, the anti-CFHR4 LCDR1 comprises the amino acid sequence of SEQ ID NO: 209; the anti-CFHR4 LCDR2 comprises the amino acid sequence of SEQ ID NO: 219; and the anti-CFHR4 LCDR3 comprises the amino acid sequence of SEQ ID NO: 229. In some embodiments, the anti-CFHR4 LCDR1 comprises the amino acid sequence of SEQ ID NO: 210; the anti-CFHR4 LCDR2 comprises the amino acid sequence of SEQ ID NO: 220; and the anti-CFHR4 LCDR3 comprises the amino acid sequence of SEQ ID NO: 230. In some embodiments, the anti-CFHR4 LCDR1 comprises the amino acid sequence of SEQ ID NO: 211; the anti-CFHR4 LCDR2 comprises the amino acid sequence of SEQ ID NO: 221; and the anti-CFHR4 LCDR3 comprises the amino acid sequence of SEQ ID NO: 231. In some embodiments, the anti-CFHR4 LCDR1 comprises the amino acid sequence of SEQ ID NO: 212; the anti-CFHR4 LCDR2 comprises the amino acid sequence of SEQ ID NO: 222; and the anti-CFHR4 LCDR3 comprises the amino acid sequence of SEQ ID NO: 232. In some embodiments, the anti-CFHR4 LCDR1 comprises the amino acid sequence of SEQ ID NO: 213; the anti-CFHR4 LCDR2 comprises the amino acid sequence of SEQ ID NO: 223; and the anti-CFHR4 LCDR3 comprises the amino acid sequence of SEQ ID NO: 233. In some embodiments, the anti-CFHR4 LCDR1 comprises the amino acid sequence of SEQ ID NO: 214; the anti-CFHR4 LCDR2 comprises the amino acid sequence of SEQ ID NO: 224; and the anti-CFHR4 LCDR3 comprises the amino acid sequence of SEQ ID NO: 234. In some embodiments, the anti-CFHR4 LCDR1 comprises the amino acid sequence of SEQ ID NO: 215; the anti-CFHR4 LCDR2 comprises the amino acid sequence of SEQ ID NO: 225; and the anti-CFHR4 LCDR3 comprises the amino acid sequence of SEQ ID NO: 235. In some embodiments, the anti-CFHR4 LCDR1 comprises the amino acid sequence of SEQ ID NO: 237; the anti-CFHR4 LCDR2 comprises the amino acid sequence of SEQ ID NO: 245; and the anti-CFHR4 LCDR3 comprises the amino acid sequence of SEQ ID NO: 253. In some embodiments, the anti-CFHR4 LCDR1 comprises the amino acid sequence of SEQ ID NO: 238; the anti-CFHR4 LCDR2 comprises the amino acid sequence of SEQ ID NO: 246; and the anti-CFHR4 LCDR3 comprises the amino acid sequence of SEQ ID NO: 254. In some embodiments, the anti-CFHR4 LCDR1 comprises the amino acid sequence of SEQ ID NO: 239; the anti-CFHR4 LCDR2 comprises the amino acid sequence of SEQ ID NO: 247; and the anti-CFHR4 LCDR3 comprises the amino acid sequence of SEQ ID NO: 255. In some embodiments, the anti-CFHR4 LCDR1 comprises the amino acid sequence of SEQ ID NO: 240; the anti-CFHR4 LCDR2 comprises the amino acid sequence of SEQ ID NO: 248; and the anti-CFHR4 LCDR3 comprises the amino acid sequence of SEQ ID NO: 256. In some embodiments, the anti-CFHR4 LCDR1 comprises the amino acid sequence of SEQ ID NO: 241; the anti-CFHR4 LCDR2 comprises the amino acid sequence of SEQ ID NO: 249; and the anti-CFHR4 LCDR3 comprises the amino acid sequence of SEQ ID NO: 257. In some embodiments, the anti-CFHR4 LCDR1 comprises the amino acid sequence of SEQ ID NO: 242; the anti-CFHR4 LCDR2 comprises the amino acid sequence of SEQ ID NO: 250; and the anti-CFHR4 LCDR3 comprises the amino acid sequence of SEQ ID NO: 258. In some embodiments, the anti-CFHR4 LCDR1 comprises the amino acid sequence of SEQ ID NO: 243; the anti-CFHR4 LCDR2 comprises the amino acid sequence of SEQ ID NO: 251; and the anti-CFHR4 LCDR3 comprises the amino acid sequence of SEQ ID NO: 259.

In some embodiments, the bispecific antibodies of the present disclosure comprise an anti-C3 VH that comprises an amino acid sequence having at least 90% (e.g., at least 90%, 91%, 92%, 93%, 94%, 95%, 96%, 97%, 98%, 99%, or 100%) sequence identity to at least one of SEQ ID NOs: 1127-1200. In some embodiments, the bispecific antibodies of the present disclosure comprise an anti-C3 VL that comprises an amino acid sequence having at least 90% (e.g., at least 90%, 91%, 92%, 93%, 94%, 95%, 96%, 97%, 98%, 99%, or 100%) sequence identity to at least one of SEQ ID NOs: 1275-1348.

In accordance with these embodiments, the anti-C3 HCDR1 comprises the amino acid sequence of SEQ ID NO: 583; the anti-C3 HCDR2 comprises the amino acid sequence of SEQ ID NO: 602; and the anti-C3 HCDR3 comprises the amino acid sequence of SEQ ID NO: 620. In some embodiments, the anti-C3 HCDR1 comprises the amino acid sequence of SEQ ID NO: 584; the anti-C3 HCDR2 comprises the amino acid sequence of SEQ ID NO: 603; and the anti-C3 HCDR3 comprises the amino acid sequence of SEQ ID NO: 621. In some embodiments, the anti-C3 HCDR1 comprises the amino acid sequence of SEQ ID NO: 585; the anti-C3 HCDR2 comprises the amino acid sequence of SEQ ID NO: 604; and the anti-C3 HCDR3 comprises the amino acid sequence of SEQ ID NO: 622. In some embodiments, the anti-C3 HCDR1 comprises the amino acid sequence of SEQ ID NO: 586; the anti-C3 HCDR2 comprises the amino acid sequence of SEQ ID NO: 605; and the anti-C3 HCDR3 comprises the amino acid sequence of SEQ ID NO: 623. In some embodiments, the anti-C3 HCDR1 comprises the amino acid sequence of SEQ ID NO: 587; the anti-C3 HCDR2 comprises the amino acid sequence of SEQ ID NO: 606; and the anti-C3 HCDR3 comprises the amino acid sequence of SEQ ID NO: 624. In some embodiments, the anti-C3 HCDR1 comprises the amino acid sequence of SEQ ID NO: 588; the anti-C3 HCDR2 comprises the amino acid sequence of SEQ ID NO: 607; and the anti-C3 HCDR3 comprises the amino acid sequence of SEQ ID NO: 625. In some embodiments, the anti-C3 HCDR1 comprises the amino acid sequence of SEQ ID NO: 589; the anti-C3 HCDR2 comprises the amino acid sequence of SEQ ID NO: 608; and the anti-C3 HCDR3 comprises the amino acid sequence of SEQ ID NO: 626. In some embodiments, the anti-C3 HCDR1 comprises the amino acid sequence of SEQ ID NO: 590; the anti-C3 HCDR2 comprises the amino acid sequence of SEQ ID NO: 609; and the anti-C3 HCDR3 comprises the amino acid sequence of SEQ ID NO: 627. In some embodiments, the anti-C3 HCDR1 comprises the amino acid sequence of SEQ ID NO: 591; the anti-C3 HCDR2 comprises the amino acid sequence of SEQ ID NO: 610; and the anti-C3 HCDR3 comprises the amino acid sequence of SEQ ID NO: 628. In some embodiments, the anti-C3 HCDR1 comprises the amino acid sequence of SEQ ID NO: 592; the anti-C3 HCDR2 comprises the amino acid sequence of SEQ ID NO: 611; and the anti-C3 HCDR3 comprises the amino acid sequence of SEQ ID NO: 629. In some embodiments, the anti-C3 HCDR1 comprises the amino acid sequence of SEQ ID NO: 593; the anti-C3 HCDR2 comprises the amino acid sequence of SEQ ID NO: 612; and the anti-C3 HCDR3 comprises the amino acid sequence of SEQ ID NO: 630. In some embodiments, the anti-C3 HCDR1 comprises the amino acid sequence of SEQ ID NO: 594; the anti-C3 HCDR2 comprises the amino acid sequence of SEQ ID NO: 613; and the anti-C3 HCDR3 comprises the amino acid sequence of SEQ ID NO: 631. In some embodiments, the anti-C3 HCDR1 comprises the amino acid sequence of SEQ ID NO: 595; the anti-C3 HCDR2 comprises the amino acid sequence of SEQ ID NO: 614; and the anti-C3 HCDR3 comprises the amino acid sequence of SEQ ID NO: 632. In some embodiments, the anti-C3 HCDR1 comprises the amino acid sequence of SEQ ID NO: 596; the anti-C3 HCDR2 comprises the amino acid sequence of SEQ ID NO: 615; and the anti-C3 HCDR3 comprises the amino acid sequence of SEQ ID NO: 633. In some embodiments, the anti-C3 HCDR1 comprises the amino acid sequence of SEQ ID NO: 597; the anti-C3 HCDR2 comprises the amino acid sequence of SEQ ID NO: 616; and the anti-C3 HCDR3 comprises the amino acid sequence of SEQ ID NO: 634. In some embodiments, the anti-C3 HCDR1 comprises the amino acid sequence of SEQ ID NO: 598; the anti-C3 HCDR2 comprises the amino acid sequence of SEQ ID NO: 617; and the anti-C3 HCDR3 comprises the amino acid sequence of SEQ ID NO: 635. In some embodiments, the anti-C3 HCDR1 comprises the amino acid sequence of SEQ ID NO: 599; the anti-C3 HCDR2 comprises the amino acid sequence of SEQ ID NO: 618; and the anti-C3 HCDR3 comprises the amino acid sequence of SEQ ID NO: 636. In some embodiments, the anti-C3 HCDR1 comprises the amino acid sequence of SEQ ID NO: 600; the anti-C3 HCDR2 comprises the amino acid sequence of SEQ ID NO: 619; and the anti-C3 HCDR3 comprises the amino acid sequence of SEQ ID NO: 637. In some embodiments, the anti-C3 HCDR1 comprises the amino acid sequence of SEQ ID NO: 639; the anti-C3 HCDR2 comprises the amino acid sequence of SEQ ID NO: 652; and the anti-C3 HCDR3 comprises the amino acid sequence of SEQ ID NO: 664. In some embodiments, the anti-C3 HCDR1 comprises the amino acid sequence of SEQ ID NO: 640; the anti-C3 HCDR2 comprises the amino acid sequence of SEQ ID NO: 653; and the anti-C3 HCDR3 comprises the amino acid sequence of SEQ ID NO: 665. In some embodiments, the anti-C3 HCDR1 comprises the amino acid sequence of SEQ ID NO: 641; the anti-C3 HCDR2 comprises the amino acid sequence of SEQ ID NO: 654; and the anti-C3 HCDR3 comprises the amino acid sequence of SEQ ID NO: 666. In some embodiments, the anti-C3 HCDR1 comprises the amino acid sequence of SEQ ID NO: 642; the anti-C3 HCDR2 comprises the amino acid sequence of SEQ ID NO: 655; and the anti-C3 HCDR3 comprises the amino acid sequence of SEQ ID NO: 667. In some embodiments, the anti-C3 HCDR1 comprises the amino acid sequence of SEQ ID NO: 643; the anti-C3 HCDR2 comprises the amino acid sequence of SEQ ID NO: 656; and the anti-C3 HCDR3 comprises the amino acid sequence of SEQ ID NO: 668. In some embodiments, the anti-C3 HCDR1 comprises the amino acid sequence of SEQ ID NO: 644; the anti-C3 HCDR2 comprises the amino acid sequence of SEQ ID NO: 657; and the anti-C3 HCDR3 comprises the amino acid sequence of SEQ ID NO: 669. In some embodiments, the anti-C3 HCDR1 comprises the amino acid sequence of SEQ ID NO: 645; the anti-C3 HCDR2 comprises the amino acid sequence of SEQ ID NO: 658; and the anti-C3 HCDR3 comprises the amino acid sequence of SEQ ID NO: 670. In some embodiments, the anti-C3 HCDR1 comprises the amino acid sequence of SEQ ID NO: 646; the anti-C3 HCDR2 comprises the amino acid sequence of SEQ ID NO: 659; and the anti-C3 HCDR3 comprises the amino acid sequence of SEQ ID NO: 671. In some embodiments, the anti-C3 HCDR1 comprises the amino acid sequence of SEQ ID NO: 647; the anti-C3 HCDR2 comprises the amino acid sequence of SEQ ID NO: 660; and the anti-C3 HCDR3 comprises the amino acid sequence of SEQ ID NO: 672. In some embodiments, the anti-C3 HCDR1 comprises the amino acid sequence of SEQ ID NO: 648; the anti-C3 HCDR2 comprises the amino acid sequence of SEQ ID NO: 661; and the anti-C3 HCDR3 comprises the amino acid sequence of SEQ ID NO: 673. In some embodiments, the anti-C3 HCDR1 comprises the amino acid sequence of SEQ ID NO: 649; the anti-C3 HCDR2 comprises the amino acid sequence of SEQ ID NO: 662; and the anti-C3 HCDR3 comprises the amino acid sequence of SEQ ID NO: 674. In some embodiments, the anti-C3 HCDR1 comprises the amino acid sequence of SEQ ID NO: 650; the anti-C3 HCDR2 comprises the amino acid sequence of SEQ ID NO: 663; and the anti-C3 HCDR3 comprises the amino acid sequence of SEQ ID NO: 675. In some embodiments, the anti-C3 HCDR1 comprises the amino acid sequence of SEQ ID NO: 677; the anti-C3 HCDR2 comprises the amino acid sequence of SEQ ID NO: 706; and the anti-C3 HCDR3 comprises the amino acid sequence of SEQ ID NO: 734. In some embodiments, the anti-C3 HCDR1 comprises the amino acid sequence of SEQ ID NO: 678; the anti-C3 HCDR2 comprises the amino acid sequence of SEQ ID NO: 707; and the anti-C3 HCDR3 comprises the amino acid sequence of SEQ ID NO: 735. In some embodiments, the anti-C3 HCDR1 comprises the amino acid sequence of SEQ ID NO: 679; the anti-C3 HCDR2 comprises the amino acid sequence of SEQ ID NO: 708; and the anti-C3 HCDR3 comprises the amino acid sequence of SEQ ID NO: 736. In some embodiments, the anti-C3 HCDR1 comprises the amino acid sequence of SEQ ID NO: 680; the anti-C3 HCDR2 comprises the amino acid sequence of SEQ ID NO: 709; and the anti-C3 HCDR3 comprises the amino acid sequence of SEQ ID NO: 737. In some embodiments, the anti-C3 HCDR1 comprises the amino acid sequence of SEQ ID NO: 681; the anti-C3 HCDR2 comprises the amino acid sequence of SEQ ID NO: 710; and the anti-C3 HCDR3 comprises the amino acid sequence of SEQ ID NO: 738. In some embodiments, the anti-C3 HCDR1 comprises the amino acid sequence of SEQ ID NO: 682; the anti-C3 HCDR2 comprises the amino acid sequence of SEQ ID NO: 711; and the anti-C3 HCDR3 comprises the amino acid sequence of SEQ ID NO: 739. In some embodiments, the anti-C3 HCDR1 comprises the amino acid sequence of SEQ ID NO: 683; the anti-C3 HCDR2 comprises the amino acid sequence of SEQ ID NO: 712; and the anti-C3 HCDR3 comprises the amino acid sequence of SEQ ID NO: 740. In some embodiments, the anti-C3 HCDR1 comprises the amino acid sequence of SEQ ID NO: 684; the anti-C3 HCDR2 comprises the amino acid sequence of SEQ ID NO: 713; and the anti-C3 HCDR3 comprises the amino acid sequence of SEQ ID NO: 741. In some embodiments, the anti-C3 HCDR1 comprises the amino acid sequence of SEQ ID NO: 685; the anti-C3 HCDR2 comprises the amino acid sequence of SEQ ID NO: 714; and the anti-C3 HCDR3 comprises the amino acid sequence of SEQ ID NO: 742. In some embodiments, the anti-C3 HCDR1 comprises the amino acid sequence of SEQ ID NO: 686; the anti-C3 HCDR2 comprises the amino acid sequence of SEQ ID NO: 715; and the anti-C3 HCDR3 comprises the amino acid sequence of SEQ ID NO: 743. In some embodiments, the anti-C3 HCDR1 comprises the amino acid sequence of SEQ ID NO: 687; the anti-C3 HCDR2 comprises the amino acid sequence of SEQ ID NO: 716; and the anti-C3 HCDR3 comprises the amino acid sequence of SEQ ID NO: 744. In some embodiments, the anti-C3 HCDR1 comprises the amino acid sequence of SEQ ID NO: 688; the anti-C3 HCDR2 comprises the amino acid sequence of SEQ ID NO: 717; and the anti-C3 HCDR3 comprises the amino acid sequence of SEQ ID NO: 745. In some embodiments, the anti-C3 HCDR1 comprises the amino acid sequence of SEQ ID NO: 689; the anti-C3 HCDR2 comprises the amino acid sequence of SEQ ID NO: 718; and the anti-C3 HCDR3 comprises the amino acid sequence of SEQ ID NO: 746. In some embodiments, the anti-C3 HCDR1 comprises the amino acid sequence of SEQ ID NO: 690; the anti-C3 HCDR2 comprises the amino acid sequence of SEQ ID NO: 719; and the anti-C3 HCDR3 comprises the amino acid sequence of SEQ ID NO: 747. In some embodiments, the anti-C3 HCDR1 comprises the amino acid sequence of SEQ ID NO: 691; the anti-C3 HCDR2 comprises the amino acid sequence of SEQ ID NO: 720; and the anti-C3 HCDR3 comprises the amino acid sequence of SEQ ID NO: 748. In some embodiments, the anti-C3 HCDR1 comprises the amino acid sequence of SEQ ID NO: 692; the anti-C3 HCDR2 comprises the amino acid sequence of SEQ ID NO: 721; and the anti-C3 HCDR3 comprises the amino acid sequence of SEQ ID NO: 749. In some embodiments, the anti-C3 HCDR1 comprises the amino acid sequence of SEQ ID NO: 693; the anti-C3 HCDR2 comprises the amino acid sequence of SEQ ID NO: 722; and the anti-C3 HCDR3 comprises the amino acid sequence of SEQ ID NO: 750. In some embodiments, the anti-C3 HCDR1 comprises the amino acid sequence of SEQ ID NO: 694; the anti-C3 HCDR2 comprises the amino acid sequence of SEQ ID NO: 723; and the anti-C3 HCDR3 comprises the amino acid sequence of SEQ ID NO: 751. In some embodiments, the anti-C3 HCDR1 comprises the amino acid sequence of SEQ ID NO: 695; the anti-C3 HCDR2 comprises the amino acid sequence of SEQ ID NO: 724; and the anti-C3 HCDR3 comprises the amino acid sequence of SEQ ID NO: 752. In some embodiments, the anti-C3 HCDR1 comprises the amino acid sequence of SEQ ID NO: 696; the anti-C3 HCDR2 comprises the amino acid sequence of SEQ ID NO: 725; and the anti-C3 HCDR3 comprises the amino acid sequence of SEQ ID NO: 753. In some embodiments, the anti-C3 HCDR1 comprises the amino acid sequence of SEQ ID NO: 697; the anti-C3 HCDR2 comprises the amino acid sequence of SEQ ID NO: 726; and the anti-C3 HCDR3 comprises the amino acid sequence of SEQ ID NO: 754. In some embodiments, the anti-C3 HCDR1 comprises the amino acid sequence of SEQ ID NO: 698; the anti-C3 HCDR2 comprises the amino acid sequence of SEQ ID NO: 727; and the anti-C3 HCDR3 comprises the amino acid sequence of SEQ ID NO: 755. In some embodiments, the anti-C3 HCDR1 comprises the amino acid sequence of SEQ ID NO: 699; the anti-C3 HCDR2 comprises the amino acid sequence of SEQ ID NO: 728; and the anti-C3 HCDR3 comprises the amino acid sequence of SEQ ID NO: 756. In some embodiments, the anti-C3 HCDR1 comprises the amino acid sequence of SEQ ID NO: 700; the anti-C3 HCDR2 comprises the amino acid sequence of SEQ ID NO: 729; and the anti-C3 HCDR3 comprises the amino acid sequence of SEQ ID NO: 757. In some embodiments, the anti-C3 HCDR1 comprises the amino acid sequence of SEQ ID NO: 701; the anti-C3 HCDR2 comprises the amino acid sequence of SEQ ID NO: 730; and the anti-C3 HCDR3 comprises the amino acid sequence of SEQ ID NO: 758. In some embodiments, the anti-C3 HCDR1 comprises the amino acid sequence of SEQ ID NO: 702; the anti-C3 HCDR2 comprises the amino acid sequence of SEQ ID NO: 731; and the anti-C3 HCDR3 comprises the amino acid sequence of SEQ ID NO: 759. In some embodiments, the anti-C3 HCDR1 comprises the amino acid sequence of SEQ ID NO: 703; the anti-C3 HCDR2 comprises the amino acid sequence of SEQ ID NO: 732; and the anti-C3 HCDR3 comprises the amino acid sequence of SEQ ID NO: 760. In some embodiments, the anti-C3 HCDR1 comprises the amino acid sequence of SEQ ID NO: 704; the anti-C3 HCDR2 comprises the amino acid sequence of SEQ ID NO: 733; and the anti-C3 HCDR3 comprises the amino acid sequence of SEQ ID NO: 761. In some embodiments, the anti-C3 HCDR1 comprises the amino acid sequence of SEQ ID NO: 763; the anti-C3 HCDR2 comprises the amino acid sequence of SEQ ID NO: 771; and the anti-C3 HCDR3 comprises the amino acid sequence of SEQ ID NO: 778. In some embodiments, the anti-C3 HCDR1 comprises the amino acid sequence of SEQ ID NO: 764; the anti-C3 HCDR2 comprises the amino acid sequence of SEQ ID NO: 772; and the anti-C3 HCDR3 comprises the amino acid sequence of SEQ ID NO: 779. In some embodiments, the anti-C3 HCDR1 comprises the amino acid sequence of SEQ ID NO: 765; the anti-C3 HCDR2 comprises the amino acid sequence of SEQ ID NO: 773; and the anti-C3 HCDR3 comprises the amino acid sequence of SEQ ID NO: 780. In some embodiments, the anti-C3 HCDR1 comprises the amino acid sequence of SEQ ID NO: 766; the anti-C3 HCDR2 comprises the amino acid sequence of SEQ ID NO: 774; and the anti-C3 HCDR3 comprises the amino acid sequence of SEQ ID NO: 781. In some embodiments, the anti-C3 HCDR1 comprises the amino acid sequence of SEQ ID NO: 767; the anti-C3 HCDR2 comprises the amino acid sequence of SEQ ID NO: 775; and the anti-C3 HCDR3 comprises the amino acid sequence of SEQ ID NO: 782. In some embodiments, the anti-C3 HCDR1 comprises the amino acid sequence of SEQ ID NO: 768; the anti-C3 HCDR2 comprises the amino acid sequence of SEQ ID NO: 776; and the anti-C3 HCDR3 comprises the amino acid sequence of SEQ ID NO: 783. In some embodiments, the anti-C3 HCDR1 comprises the amino acid sequence of SEQ ID NO: 769; the anti-C3 HCDR2 comprises the amino acid sequence of SEQ ID NO: 777; and the anti-C3 HCDR3 comprises the amino acid sequence of SEQ ID NO: 784. In some embodiments, the anti-C3 HCDR1 comprises the amino acid sequence of SEQ ID NO: 786; the anti-C3 HCDR2 comprises the amino acid sequence of SEQ ID NO: 794; and the anti-C3 HCDR3 comprises the amino acid sequence of SEQ ID NO: 801. In some embodiments, the anti-C3 HCDR1 comprises the amino acid sequence of SEQ ID NO: 787; the anti-C3 HCDR2 comprises the amino acid sequence of SEQ ID NO: 795; and the anti-C3 HCDR3 comprises the amino acid sequence of SEQ ID NO: 802. In some embodiments, the anti-C3 HCDR1 comprises the amino acid sequence of SEQ ID NO: 788; the anti-C3 HCDR2 comprises the amino acid sequence of SEQ ID NO: 796; and the anti-C3 HCDR3 comprises the amino acid sequence of SEQ ID NO: 803. In some embodiments, the anti-C3 HCDR1 comprises the amino acid sequence of SEQ ID NO: 789; the anti-C3 HCDR2 comprises the amino acid sequence of SEQ ID NO: 797; and the anti-C3 HCDR3 comprises the amino acid sequence of SEQ ID NO: 804. In some embodiments, the anti-C3 HCDR1 comprises the amino acid sequence of SEQ ID NO: 790; the anti-C3 HCDR2 comprises the amino acid sequence of SEQ ID NO: 798; and the anti-C3 HCDR3 comprises the amino acid sequence of SEQ ID NO: 805. In some embodiments, the anti-C3 HCDR1 comprises the amino acid sequence of SEQ ID NO: 791; the anti-C3 HCDR2 comprises the amino acid sequence of SEQ ID NO: 799; and the anti-C3 HCDR3 comprises the amino acid sequence of SEQ ID NO: 806. In some embodiments, the anti-C3 HCDR1 comprises the amino acid sequence of SEQ ID NO: 792; the anti-C3 HCDR2 comprises the amino acid sequence of SEQ ID NO: 800; and the anti-C3 HCDR3 comprises the amino acid sequence of SEQ ID NO: 807. In some embodiments, the anti-C3 HCDR1 comprises the amino acid sequence of SEQ ID NO: 809; the anti-C3 HCDR2 comprises the amino acid sequence of SEQ ID NO: 812; and the anti-C3 HCDR3 comprises the amino acid sequence of SEQ ID NO: 815. In some embodiments, the anti-C3 HCDR1 comprises the amino acid sequence of SEQ ID NO: 810; the anti-C3 HCDR2 comprises the amino acid sequence of SEQ ID NO: 813; and the anti-C3 HCDR3 comprises the amino acid sequence of SEQ ID NO: 816.

In some embodiments, the anti-C3 LCDR1 comprises the amino acid sequence of SEQ ID NO: 818; the anti-C3 LCDR2 comprises the amino acid sequence of SEQ ID NO: 850; and the anti-C3 LCDR3 comprises the amino acid sequence of SEQ ID NO: 881. In some embodiments, the anti-C3 LCDR1 comprises the amino acid sequence of SEQ ID NO: 819; the anti-C3 LCDR2 comprises the amino acid sequence of SEQ ID NO: 851; and the anti-C3 LCDR3 comprises the amino acid sequence of SEQ ID NO: 882. In some embodiments, the anti-C3 LCDR1 comprises the amino acid sequence of SEQ ID NO: 820; the anti-C3 LCDR2 comprises the amino acid sequence of SEQ ID NO: 852; and the anti-C3 LCDR3 comprises the amino acid sequence of SEQ ID NO: 883. In some embodiments, the anti-C3 LCDR1 comprises the amino acid sequence of SEQ ID NO: 821; the anti-C3 LCDR2 comprises the amino acid sequence of SEQ ID NO: 853; and the anti-C3 LCDR3 comprises the amino acid sequence of SEQ ID NO: 884. In some embodiments, the anti-C3 LCDR1 comprises the amino acid sequence of SEQ ID NO: 822; the anti-C3 LCDR2 comprises the amino acid sequence of SEQ ID NO: 854; and the anti-C3 LCDR3 comprises the amino acid sequence of SEQ ID NO: 885. In some embodiments, the anti-C3 LCDR1 comprises the amino acid sequence of SEQ ID NO: 823; the anti-C3 LCDR2 comprises the amino acid sequence of SEQ ID NO: 855; and the anti-C3 LCDR3 comprises the amino acid sequence of SEQ ID NO: 886. In some embodiments, the anti-C3 LCDR1 comprises the amino acid sequence of SEQ ID NO: 824; the anti-C3 LCDR2 comprises the amino acid sequence of SEQ ID NO: 856; and the anti-C3 LCDR3 comprises the amino acid sequence of SEQ ID NO: 887. In some embodiments, the anti-C3 LCDR1 comprises the amino acid sequence of SEQ ID NO: 825; the anti-C3 LCDR2 comprises the amino acid sequence of SEQ ID NO: 857; and the anti-C3 LCDR3 comprises the amino acid sequence of SEQ ID NO: 888. In some embodiments, the anti-C3 LCDR1 comprises the amino acid sequence of SEQ ID NO: 826; the anti-C3 LCDR2 comprises the amino acid sequence of SEQ ID NO: 858; and the anti-C3 LCDR3 comprises the amino acid sequence of SEQ ID NO: 889. In some embodiments, the anti-C3 LCDR1 comprises the amino acid sequence of SEQ ID NO: 827; the anti-C3 LCDR2 comprises the amino acid sequence of SEQ ID NO: 859; and the anti-C3 LCDR3 comprises the amino acid sequence of SEQ ID NO: 890. In some embodiments, the anti-C3 LCDR1 comprises the amino acid sequence of SEQ ID NO: 828; the anti-C3 LCDR2 comprises the amino acid sequence of SEQ ID NO: 860; and the anti-C3 LCDR3 comprises the amino acid sequence of SEQ ID NO: 891. In some embodiments, the anti-C3 LCDR1 comprises the amino acid sequence of SEQ ID NO: 829; the anti-C3 LCDR2 comprises the amino acid sequence of SEQ ID NO: 861; and the anti-C3 LCDR3 comprises the amino acid sequence of SEQ ID NO: 892. In some embodiments, the anti-C3 LCDR1 comprises the amino acid sequence of SEQ ID NO: 830; the anti-C3 LCDR2 comprises the amino acid sequence of SEQ ID NO: 862; and the anti-C3 LCDR3 comprises the amino acid sequence of SEQ ID NO: 893. In some embodiments, the anti-C3 LCDR1 comprises the amino acid sequence of SEQ ID NO: 831; the anti-C3 LCDR2 comprises the amino acid sequence of SEQ ID NO: 863; and the anti-C3 LCDR3 comprises the amino acid sequence of SEQ ID NO: 894. In some embodiments, the anti-C3 LCDR1 comprises the amino acid sequence of SEQ ID NO: 832; the anti-C3 LCDR2 comprises the amino acid sequence of SEQ ID NO: 864; and the anti-C3 LCDR3 comprises the amino acid sequence of SEQ ID NO: 895. In some embodiments, the anti-C3 LCDR1 comprises the amino acid sequence of SEQ ID NO: 833; the anti-C3 LCDR2 comprises the amino acid sequence of SEQ ID NO: 865; and the anti-C3 LCDR3 comprises the amino acid sequence of SEQ ID NO: 896. In some embodiments, the anti-C3 LCDR1 comprises the amino acid sequence of SEQ ID NO: 834; the anti-C3 LCDR2 comprises the amino acid sequence of SEQ ID NO: 866; and the anti-C3 LCDR3 comprises the amino acid sequence of SEQ ID NO: 897. In some embodiments, the anti-C3 LCDR1 comprises the amino acid sequence of SEQ ID NO: 835; the anti-C3 LCDR2 comprises the amino acid sequence of SEQ ID NO: 867; and the anti-C3 LCDR3 comprises the amino acid sequence of SEQ ID NO: 898. In some embodiments, the anti-C3 LCDR1 comprises the amino acid sequence of SEQ ID NO: 836; the anti-C3 LCDR2 comprises the amino acid sequence of SEQ ID NO: 868; and the anti-C3 LCDR3 comprises the amino acid sequence of SEQ ID NO: 899. In some embodiments, the anti-C3 LCDR1 comprises the amino acid sequence of SEQ ID NO: 837; the anti-C3 LCDR2 comprises the amino acid sequence of SEQ ID NO: 869; and the anti-C3 LCDR3 comprises the amino acid sequence of SEQ ID NO: 900. In some embodiments, the anti-C3 LCDR1 comprises the amino acid sequence of SEQ ID NO: 838; the anti-C3 LCDR2 comprises the amino acid sequence of SEQ ID NO: 870; and the anti-C3 LCDR3 comprises the amino acid sequence of SEQ ID NO: 901. In some embodiments, the anti-C3 LCDR1 comprises the amino acid sequence of SEQ ID NO: 839; the anti-C3 LCDR2 comprises the amino acid sequence of SEQ ID NO: 871; and the anti-C3 LCDR3 comprises the amino acid sequence of SEQ ID NO: 902. In some embodiments, the anti-C3 LCDR1 comprises the amino acid sequence of SEQ ID NO: 840; the anti-C3 LCDR2 comprises the amino acid sequence of SEQ ID NO: 872; and the anti-C3 LCDR3 comprises the amino acid sequence of SEQ ID NO: 903. In some embodiments, the anti-C3 LCDR1 comprises the amino acid sequence of SEQ ID NO: 841; the anti-C3 LCDR2 comprises the amino acid sequence of SEQ ID NO: 873; and the anti-C3 LCDR3 comprises the amino acid sequence of SEQ ID NO: 904. In some embodiments, the anti-C3 LCDR1 comprises the amino acid sequence of SEQ ID NO: 842; the anti-C3 LCDR2 comprises the amino acid sequence of SEQ ID NO: 874; and the anti-C3 LCDR3 comprises the amino acid sequence of SEQ ID NO: 905. In some embodiments, the anti-C3 LCDR1 comprises the amino acid sequence of SEQ ID NO: 843; the anti-C3 LCDR2 comprises the amino acid sequence of SEQ ID NO: 875; and the anti-C3 LCDR3 comprises the amino acid sequence of SEQ ID NO: 906. In some embodiments, the anti-C3 LCDR1 comprises the amino acid sequence of SEQ ID NO: 844; the anti-C3 LCDR2 comprises the amino acid sequence of SEQ ID NO: 876; and the anti-C3 LCDR3 comprises the amino acid sequence of SEQ ID NO: 907. In some embodiments, the anti-C3 LCDR1 comprises the amino acid sequence of SEQ ID NO: 845; the anti-C3 LCDR2 comprises the amino acid sequence of SEQ ID NO: 877; and the anti-C3 LCDR3 comprises the amino acid sequence of SEQ ID NO: 908. In some embodiments, the anti-C3 LCDR1 comprises the amino acid sequence of SEQ ID NO: 846; the anti-C3 LCDR2 comprises the amino acid sequence of SEQ ID NO: 878; and the anti-C3 LCDR3 comprises the amino acid sequence of SEQ ID NO: 909. In some embodiments, the anti-C3 LCDR1 comprises the amino acid sequence of SEQ ID NO: 847; the anti-C3 LCDR2 comprises the amino acid sequence of SEQ ID NO: 879; and the anti-C3 LCDR3 comprises the amino acid sequence of SEQ ID NO: 910. In some embodiments, the anti-C3 LCDR1 comprises the amino acid sequence of SEQ ID NO: 848; the anti-C3 LCDR2 comprises the amino acid sequence of SEQ ID NO: 880; and the anti-C3 LCDR3 comprises the amino acid sequence of SEQ ID NO: 911. In some embodiments, the anti-C3 LCDR1 comprises the amino acid sequence of SEQ ID NO: 913; the anti-C3 LCDR2 comprises the amino acid sequence of SEQ ID NO: 916; and the anti-C3 LCDR3 comprises the amino acid sequence of SEQ ID NO: 919. In some embodiments, the anti-C3 LCDR1 comprises the amino acid sequence of SEQ ID NO: 914; the anti-C3 LCDR2 comprises the amino acid sequence of SEQ ID NO: 917; and the anti-C3 LCDR3 comprises the amino acid sequence of SEQ ID NO: 920. In some embodiments, the anti-C3 LCDR1 comprises the amino acid sequence of SEQ ID NO: 914; the anti-C3 LCDR2 comprises the amino acid sequence of SEQ ID NO: 917; and the anti-C3 LCDR3 comprises the amino acid sequence of SEQ ID NO: 920. In some embodiments, the anti-C3 LCDR1 comprises the amino acid sequence of SEQ ID NO: 922; the anti-C3 LCDR2 comprises the amino acid sequence of SEQ ID NO: 931; and the anti-C3 LCDR3 comprises the amino acid sequence of SEQ ID NO: 940. In some embodiments, the anti-C3 LCDR1 comprises the amino acid sequence of SEQ ID NO: 923; the anti-C3 LCDR2 comprises the amino acid sequence of SEQ ID NO: 932; and the anti-C3 LCDR3 comprises the amino acid sequence of SEQ ID NO: 941. In some embodiments, the anti-C3 LCDR1 comprises the amino acid sequence of SEQ ID NO: 924; the anti-C3 LCDR2 comprises the amino acid sequence of SEQ ID NO: 933; and the anti-C3 LCDR3 comprises the amino acid sequence of SEQ ID NO: 942. In some embodiments, the anti-C3 LCDR1 comprises the amino acid sequence of SEQ ID NO: 925; the anti-C3 LCDR2 comprises the amino acid sequence of SEQ ID NO: 934; and the anti-C3 LCDR3 comprises the amino acid sequence of SEQ ID NO: 943. In some embodiments, the anti-C3 LCDR1 comprises the amino acid sequence of SEQ ID NO: 926; the anti-C3 LCDR2 comprises the amino acid sequence of SEQ ID NO: 935; and the anti-C3 LCDR3 comprises the amino acid sequence of SEQ ID NO: 944. In some embodiments, the anti-C3 LCDR1 comprises the amino acid sequence of SEQ ID NO: 927; the anti-C3 LCDR2 comprises the amino acid sequence of SEQ ID NO: 936; and the anti-C3 LCDR3 comprises the amino acid sequence of SEQ ID NO: 945. In some embodiments, the anti-C3 LCDR1 comprises the amino acid sequence of SEQ ID NO: 928; the anti-C3 LCDR2 comprises the amino acid sequence of SEQ ID NO: 937; and the anti-C3 LCDR3 comprises the amino acid sequence of SEQ ID NO: 946. In some embodiments, the anti-C3 LCDR1 comprises the amino acid sequence of SEQ ID NO: 929; the anti-C3 LCDR2 comprises the amino acid sequence of SEQ ID NO: 938; and the anti-C3 LCDR3 comprises the amino acid sequence of SEQ ID NO: 947. In some embodiments, the anti-C3 LCDR1 comprises the amino acid sequence of SEQ ID NO: 949; the anti-C3 LCDR2 comprises the amino acid sequence of SEQ ID NO: 984; and the anti-C3 LCDR3 comprises the amino acid sequence of SEQ ID NO: 1019. In some embodiments, the anti-C3 LCDR1 comprises the amino acid sequence of SEQ ID NO: 950; the anti-C3 LCDR2 comprises the amino acid sequence of SEQ ID NO: 985; and the anti-C3 LCDR3 comprises the amino acid sequence of SEQ ID NO: 1020. In some embodiments, the anti-C3 LCDR1 comprises the amino acid sequence of SEQ ID NO: 951; the anti-C3 LCDR2 comprises the amino acid sequence of SEQ ID NO: 986; and the anti-C3 LCDR3 comprises the amino acid sequence of SEQ ID NO: 1021. In some embodiments, the anti-C3 LCDR1 comprises the amino acid sequence of SEQ ID NO: 952; the anti-C3 LCDR2 comprises the amino acid sequence of SEQ ID NO: 987; and the anti-C3 LCDR3 comprises the amino acid sequence of SEQ ID NO: 1022. In some embodiments, the anti-C3 LCDR1 comprises the amino acid sequence of SEQ ID NO: 953; the anti-C3 LCDR2 comprises the amino acid sequence of SEQ ID NO: 988; and the anti-C3 LCDR3 comprises the amino acid sequence of SEQ ID NO: 1023. In some embodiments, the anti-C3 LCDR1 comprises the amino acid sequence of SEQ ID NO: 954; the anti-C3 LCDR2 comprises the amino acid sequence of SEQ ID NO: 989; and the anti-C3 LCDR3 comprises the amino acid sequence of SEQ ID NO: 1024. In some embodiments, the anti-C3 LCDR1 comprises the amino acid sequence of SEQ ID NO: 955; the anti-C3 LCDR2 comprises the amino acid sequence of SEQ ID NO: 990; and the anti-C3 LCDR3 comprises the amino acid sequence of SEQ ID NO: 1025. In some embodiments, the anti-C3 LCDR1 comprises the amino acid sequence of SEQ ID NO: 956; the anti-C3 LCDR2 comprises the amino acid sequence of SEQ ID NO: 991; and the anti-C3 LCDR3 comprises the amino acid sequence of SEQ ID NO: 1026. In some embodiments, the anti-C3 LCDR1 comprises the amino acid sequence of SEQ ID NO: 957; the anti-C3 LCDR2 comprises the amino acid sequence of SEQ ID NO: 992; and the anti-C3 LCDR3 comprises the amino acid sequence of SEQ ID NO: 1027. In some embodiments, the anti-C3 LCDR1 comprises the amino acid sequence of SEQ ID NO: 958; the anti-C3 LCDR2 comprises the amino acid sequence of SEQ ID NO: 993; and the anti-C3 LCDR3 comprises the amino acid sequence of SEQ ID NO: 1028. In some embodiments, the anti-C3 LCDR1 comprises the amino acid sequence of SEQ ID NO: 959; the anti-C3 LCDR2 comprises the amino acid sequence of SEQ ID NO: 994; and the anti-C3 LCDR3 comprises the amino acid sequence of SEQ ID NO: 1029. In some embodiments, the anti-C3 LCDR1 comprises the amino acid sequence of SEQ ID NO: 960; the anti-C3 LCDR2 comprises the amino acid sequence of SEQ ID NO: 995; and the anti-C3 LCDR3 comprises the amino acid sequence of SEQ ID NO: 1030. In some embodiments, the anti-C3 LCDR1 comprises the amino acid sequence of SEQ ID NO: 961; the anti-C3 LCDR2 comprises the amino acid sequence of SEQ ID NO: 996; and the anti-C3 LCDR3 comprises the amino acid sequence of SEQ ID NO: 1031. In some embodiments, the anti-C3 LCDR1 comprises the amino acid sequence of SEQ ID NO: 962; the anti-C3 LCDR2 comprises the amino acid sequence of SEQ ID NO: 997; and the anti-C3 LCDR3 comprises the amino acid sequence of SEQ ID NO: 1032. In some embodiments, the anti-C3 LCDR1 comprises the amino acid sequence of SEQ ID NO: 963; the anti-C3 LCDR2 comprises the amino acid sequence of SEQ ID NO: 998; and the anti-C3 LCDR3 comprises the amino acid sequence of SEQ ID NO: 1033. In some embodiments, the anti-C3 LCDR1 comprises the amino acid sequence of SEQ ID NO: 964; the anti-C3 LCDR2 comprises the amino acid sequence of SEQ ID NO: 999; and the anti-C3 LCDR3 comprises the amino acid sequence of SEQ ID NO: 1034. In some embodiments, the anti-C3 LCDR1 comprises the amino acid sequence of SEQ ID NO: 965; the anti-C3 LCDR2 comprises the amino acid sequence of SEQ ID NO: 1000; and the anti-C3 LCDR3 comprises the amino acid sequence of SEQ ID NO: 1035. In some embodiments, the anti-C3 LCDR1 comprises the amino acid sequence of SEQ ID NO: 966; the anti-C3 LCDR2 comprises the amino acid sequence of SEQ ID NO: 1001; and the anti-C3 LCDR3 comprises the amino acid sequence of SEQ ID NO: 1036. In some embodiments, the anti-C3 LCDR1 comprises the amino acid sequence of SEQ ID NO: 967; the anti-C3 LCDR2 comprises the amino acid sequence of SEQ ID NO: 1002; and the anti-C3 LCDR3 comprises the amino acid sequence of SEQ ID NO: 1037. In some embodiments, the anti-C3 LCDR1 comprises the amino acid sequence of SEQ ID NO: 968; the anti-C3 LCDR2 comprises the amino acid sequence of SEQ ID NO: 1003; and the anti-C3 LCDR3 comprises the amino acid sequence of SEQ ID NO: 1038. In some embodiments, the anti-C3 LCDR1 comprises the amino acid sequence of SEQ ID NO: 969; the anti-C3 LCDR2 comprises the amino acid sequence of SEQ ID NO: 1004; and the anti-C3 LCDR3 comprises the amino acid sequence of SEQ ID NO: 1039. In some embodiments, the anti-C3 LCDR1 comprises the amino acid sequence of SEQ ID NO: 970; the anti-C3 LCDR2 comprises the amino acid sequence of SEQ ID NO: 1005; and the anti-C3 LCDR3 comprises the amino acid sequence of SEQ ID NO: 1040. In some embodiments, the anti-C3 LCDR1 comprises the amino acid sequence of SEQ ID NO: 971; the anti-C3 LCDR2 comprises the amino acid sequence of SEQ ID NO: 1006; and the anti-C3 LCDR3 comprises the amino acid sequence of SEQ ID NO: 1041. In some embodiments, the anti-C3 LCDR1 comprises the amino acid sequence of SEQ ID NO: 972; the anti-C3 LCDR2 comprises the amino acid sequence of SEQ ID NO: 1007; and the anti-C3 LCDR3 comprises the amino acid sequence of SEQ ID NO: 1042. In some embodiments, the anti-C3 LCDR1 comprises the amino acid sequence of SEQ ID NO: 973; the anti-C3 LCDR2 comprises the amino acid sequence of SEQ ID NO: 1008; and the anti-C3 LCDR3 comprises the amino acid sequence of SEQ ID NO: 1043. In some embodiments, the anti-C3 LCDR1 comprises the amino acid sequence of SEQ ID NO: 974; the anti-C3 LCDR2 comprises the amino acid sequence of SEQ ID NO: 1009; and the anti-C3 LCDR3 comprises the amino acid sequence of SEQ ID NO: 1044. In some embodiments, the anti-C3 LCDR1 comprises the amino acid sequence of SEQ ID NO: 975; the anti-C3 LCDR2 comprises the amino acid sequence of SEQ ID NO: 1010; and the anti-C3 LCDR3 comprises the amino acid sequence of SEQ ID NO: 1045. In some embodiments, the anti-C3 LCDR1 comprises the amino acid sequence of SEQ ID NO: 976; the anti-C3 LCDR2 comprises the amino acid sequence of SEQ ID NO: 1011; and the anti-C3 LCDR3 comprises the amino acid sequence of SEQ ID NO: 1046. In some embodiments, the anti-C3 LCDR1 comprises the amino acid sequence of SEQ ID NO: 977; the anti-C3 LCDR2 comprises the amino acid sequence of SEQ ID NO: 1012; and the anti-C3 LCDR3 comprises the amino acid sequence of SEQ ID NO: 1047. In some embodiments, the anti-C3 LCDR1 comprises the amino acid sequence of SEQ ID NO: 978; the anti-C3 LCDR2 comprises the amino acid sequence of SEQ ID NO: 1013; and the anti-C3 LCDR3 comprises the amino acid sequence of SEQ ID NO: 1048. In some embodiments, the anti-C3 LCDR1 comprises the amino acid sequence of SEQ ID NO: 979; the anti-C3 LCDR2 comprises the amino acid sequence of SEQ ID NO: 1014; and the anti-C3 LCDR3 comprises the amino acid sequence of SEQ ID NO: 1049. In some embodiments, the anti-C3 LCDR1 comprises the amino acid sequence of SEQ ID NO: 980; the anti-C3 LCDR2 comprises the amino acid sequence of SEQ ID NO: 1015; and the anti-C3 LCDR3 comprises the amino acid sequence of SEQ ID NO: 1050. In some embodiments, the anti-C3 LCDR1 comprises the amino acid sequence of SEQ ID NO: 981; the anti-C3 LCDR2 comprises the amino acid sequence of SEQ ID NO: 1016; and the anti-C3 LCDR3 comprises the amino acid sequence of SEQ ID NO: 1051. In some embodiments, the anti-C3 LCDR1 comprises the amino acid sequence of SEQ ID NO: 982; the anti-C3 LCDR2 comprises the amino acid sequence of SEQ ID NO: 1017; and the anti-C3 LCDR3 comprises the amino acid sequence of SEQ ID NO: 1052.

In some embodiments, the bispecific antibodies of the present disclosure include an anti-C3 antigen-binding site comprises a VH and a VL that are at least 90% identical to a VH and a VL from any anti-C3 antibody. In some embodiments, a bispecific antibody of the present disclosure includes an anti-CFHR4 antigen-binding site, or an antigen-binding fragment thereof, and an anti-C3 antigen-binding site, or an antigen-binding fragment thereof. Such bispecific antibodies can be used to target the complement activation pathway from two different mechanisms, and thus provide additional therapeutic benefits. In some embodiments, the second anti-C3 antigen-binding site comprises a VH and a VL that are at least 90% identical to a VH and a VL from an NGM621 antibody (or an antigen-binding fragment thereof). NGM621 is a humanized IgG1 monoclonal antibody engineered to inhibit complement C3. In some embodiments, the second anti-C3 antigen-binding site comprises a VH and a VL that are at least 90% identical to a VH and a VL from an anti-C3 antibody, or an antigen-binding fragment thereof, disclosed in U.S. Pat. No. 9,815,890 or disclosed in PCT Application No. PCT/US2019/025123, both of which are incorporated herein by reference.

In other embodiments, the anti-CFHR4 antigen-binding site can be any of the anti-CFHR4 antibodies of the present disclosure, and the anti-C3 arm can be any C3 antagonist, including but not limited to, POT-4 (AL-78898A) and APL-2. POT-4 (AL-78898A) is a synthetic cyclic peptide conjugated to a PEG polymer that binds specifically to C3 (e.g., a compstatin analog); it was the first complement inhibitor to be tested in the treatment of macular degeneration. APL-2 (pegcetacoplan) is a symmetrical molecule comprised of two identical pentadecapeptides covalently bound to the ends of a linear 40-kDa polyethylene glycol molecule; it binds both C3 and C3b.

In some embodiments, the second anti-C3 antigen-binding site comprises a VH and a VL that are at least 90% identical to a VH and a VL from any antibody that binds Complement Component 3b (C3b). In some embodiments, the second anti-C3b antigen-binding site comprises a VH and a VL that are at least 90% identical to a VH and a VL from an S77 antibody (or an antigen-binding fragment thereof). S77 is an antibody that selectively recognizes C3b but not the non-activated molecule C3. In some embodiments, the second anti-C3 antigen-binding site comprises a VH and a VL that are at least 90% identical to a VH and a VL from an anti-C3 antibody, or an antigen-binding fragment thereof, disclosed in U.S. Pat. No. 8,012,473 or disclosed in PCT Application No. PCT/US2008/065771, both of which are incorporated herein by reference (see also, Katschke, K. J. et al., Journal of Biological Chemistry, 2009, vol. 284 (16): 10473-10479).

### Anti-C3/Anti-VEGF Bispecific Antibodies

Embodiments of the present disclosure relate to the treatment and/or prevention of age-related macular degeneration (AMD), including both neovascular AMD (“wet” AMD) and non-neovascular AMD (“dry” AMD), as well as advanced forms of non-neovascular AMD (geographic atrophy or GA). In some embodiments, the present disclosure provides novel therapeutic bispecific antibodies that target components of the alternative pathway of the complement activation system and the Vascular Endothelial Growth Factor (VEGF) signaling pathway to treat AMD. As described further herein, embodiments of the present disclosure also include a bispecific antibody comprising an antigen-binding site directed against CFHR4 or C3, and an antigen-binding site directed against VEGF. In accordance with these embodiments, the anti-CFHR4 antigen-binding site comprises a heavy chain variable region (VH) comprising complementarity determining regions (CDRs) HCDR1, HCDR2, and HCDR3 from one or more of SEQ ID NOs: 260-265, 284-293, 324-334, and 368-380; and a light chain variable region (VL) comprising complementarity determining regions (CDRs) LCDR1, LCDR2, and LCDR3 from one or more of SEQ ID NOs: 272-277, 304-313, 346-356, and 394-406; and the second anti-VEGF antigen-binding site comprises a VH and a VL that are at least 90% identical to a VH and a VL from an anti-VEGF antibody. In some embodiments, the anti-C3 antigen binding site comprises a heavy chain variable region (VH) comprising complementarity determining regions (CDRs) HCDR1, HCDR2, and HCDR3 from one or more of SEQ ID NOs: 1127-1200; and a light chain variable region (VL) comprising complementarity determining regions (CDRs) LCDR1, LCDR2, and LCDR3 from one or more of SEQ ID NOs: 1275-1348; and the second anti-VEGF antigen-binding site comprises a VH and a VL that are at least 90% identical to a VH and a VL from an anti-VEGF antibody.

In some embodiments, the second anti-VEGF antigen-binding site comprises a VH and a VL that are at least 90% identical to a VH and a VL from any anti-VEGF antibody. In some embodiments, a bispecific antibody of the present disclosure includes an anti-CFHR4 antigen-binding site, or an antigen-binding fragment thereof, and an anti-VEGF antigen-binding site, or an antigen-binding fragment thereof. Such bispecific antibodies can be used to treat AMD via inhibition of the complement activation pathway and inhibition of the VEGF signaling pathway, and thus provide additional therapeutic benefits. The VEGF antigen-binding site can be selected from any anti-VEGF antibody, including but not limited to, Bevacizumab, Ranibizumab, Brolucizumab, Faricimab, and Vanucizumab, or an antigen-binding fragment thereof.

In some embodiments, the second anti-VEGF antigen-binding site comprises a VH and a VL that are at least 90% identical to a VH and a VL from Bevacizumab (or an antigen-binding fragment thereof). Bevacizumab (Avastin) is an anti-VEGF human monoclonal antibody. In some embodiments, the second anti-VEGF antigen-binding site comprises a VH and a VL that are at least 90% identical to a VH and a VL from an anti-VEGF antibody, or an antigen-binding fragment thereof, disclosed in EP Patent Nos. EP0666868 and EP0817648, or disclosed in PCT Application No. PCT/US1992/009218, all of which are incorporated herein by reference.

In some embodiments, the second anti-VEGF antigen-binding site comprises a VH and a VL that are at least 90% identical to a VH and a VL from Ranibizumab (or an antigen-binding fragment thereof). Ranibizumab (Lucentis) is a Fab fragment derived from Bevacizumab. In some embodiments, the second anti-VEGF antigen-binding site comprises a VH and a VL that are at least 90% identical to a VH and a VL from an anti-VEGF antibody, or an antigen-binding fragment thereof, disclosed in U.S. patent application Ser. No. 11/840,279, or disclosed in PCT Application No. PCT/EP2007/007276, both of which are incorporated herein by reference.

In some embodiments, the second anti-VEGF antigen-binding site comprises a VH and a VL that are at least 90% identical to a VH and a VL from Brolucizumab (or an antigen-binding fragment thereof). Brolucizumab (RTH258; Beovu) is a humanized scFv that is an inhibitor of VEGF-A. In some embodiments, the second anti-VEGF antigen-binding site comprises a VH and a VL that are at least 90% identical to a VH and a VL from an anti-VEGF antibody, or an antigen-binding fragment thereof, disclosed in PCT Application No. PCT/US2015/059575, or disclosed in PCT Application No. PCT/US2015/059571, both of which are incorporated herein by reference.

In some embodiments, the second anti-VEGF antigen-binding site comprises a VH and a VL that are at least 90% identical to a VH and a VL from Faricimab (or an antigen-binding fragment thereof). Faricimab (RG7716) is a bispecific, monoclonal antibody specifically designed for the eye that simultaneously binds to and neutralizes both Angiopoietin-2 (Ang-2) and vascular endothelial growth factor A (VEGF-A). In some embodiments, the second anti-VEGF antigen-binding site comprises a VH and a VL that are at least 90% identical to a VH and a VL from an anti-VEGF antibody, or an antigen-binding fragment thereof, disclosed in U.S. Pat. No. 10,072,075, or disclosed in PCT Application No. PCT/US2016/053454, both of which are incorporated herein by reference.

In some embodiments, the second anti-VEGF antigen-binding site comprises a VH and a VL that are at least 90% identical to a VH and a VL from Vanucizumab (or an antigen-binding fragment thereof). Vanucizumab (RG7721) is a humanized bispecific, monoclonal antibody designed for the treatment of cancer composed of two different heavy chains and two different light chains (one arm binds Ang2 and the other binds VEGF (bevacizumab)). In some embodiments, the second anti-VEGF antigen-binding site comprises a VH and a VL that are at least 90% identical to a VH and a VL from an anti-VEGF antibody, or an antigen-binding fragment thereof, disclosed in U.S. Pat. No. 8,945,552, or disclosed in PCT Application No. PCT/EP2011/054504, both of which are incorporated herein by reference.

In other embodiments, a bispecific antibody of the present disclosure includes an anti-CFHR4 antigen binding site or an anti-C3 antigen binding site (or antigen binding fragments thereof), and an anti-VEGF antigen binding site (or an antigen binding fragment thereof) from any VEGF antagonist, including but not limited to, anti-VEGF antibodies (e.g., bevacizumab, sevacizumab, and ranibizumab), anti-VEGFR2 antibodies and related molecules (e.g., ramucirumab, tanibirumab, aflibercept), anti-VEGFR1 antibodies and related molecules (e.g., icrucumab, aflibercept (VEGF Trap-Eye; EYLEA®), and ziv-aflibercept (VEGF Trap; ZALTRAP®)), anti-VEGF arms of VEGF bispecific antibodies (e.g., MP-0250, vanucizumab (VEGF-ANG2)), including anti-VEGF, anti-VEGFR1, and anti-VEGFR2 arms.

### Multispecific Antibodies

The amino acid sequences of the bispecific antibodies of the present disclosure are not limited to the specific amino acid sequences described herein. Indeed, an anti-CFHR4/anti-C3 bispecific antibody, an anti-CFHR4/anti-VEGF bispecific antibody, and an anti-C3/anti-VEGF bispecific antibody, or antigen-binding fragments thereof, can comprise any heavy chain polypeptide or light chain polypeptide that competes with the various antigen-binding sites of these antibodies. Antibody competition can be assayed using routine peptide competition assays such as, for example, ELISA, Western blot, or immunohistochemistry methods (see, e.g., U.S. Pat. Nos. 4,828,981 and 8,568,992; and Braitbard et al., Proteome Sci., 4:12 (2006)).

A bispecific antibody of the present disclosure may be a whole antibody, or an antigen-binding fragment of a whole antibody. As defined herein, antigen-binding antibody fragments encompassed by the present disclosure include, but are not limited to, F(ab′)2, Fab′, Fab, Fv, scFv, dsFv, dAb, and single chain binding polypeptides. Antibody fragments and their therapeutic utility are further described in, e.g., Nelson, A. L., MAbs. 2010 January-February; 2 (1): 77-83; Joosten et al., Microbial Cell Factories volume 2, Article number: 1 (2003); and Bates A, Power CA., Antibodies (Basel). 2019; 8 (2): 28; doi: 10.3390/antib8020028). In some embodiments, the anti-CFHR4, the anti-C3, and/or the anti-VEGF antigen-binding fragments are a single-chain variable fragments (scFv), which are engineered antibodies generated by the fusion of the heavy (VH) and light chains (VL) of immunoglobulins through a short polypeptide linker. Single chain variable domain (Fv) fragments (scFv) are used in the art in a variety of clinical and therapeutic applications, primarily due to their improved pharmacokinetic properties as compared to the parent monoclonal antibodies and the relative ease of producing them in large quantities at low cost (Monnier et al., Antibodies 2013, 2 (2), 193-208; doi.org/10.3390/antib2020193; Safdari et al., Mol Med. 2016; 22:258-270; and Lu, R., Hwang, Y., Liu, I. et al. Development of therapeutic antibodies for the treatment of diseases. J Biomed Sci 27, 1 (2020). https://doi.org/10.1186/s12929-019-0592-z).

The bispecific antibodies of the present disclosure may be a diabody. Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003). An anti-CFHR4, anti-C3, or anti-VEGF antibody of the present disclosure may be a single-domain antibody (also referred to as a nanobody). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1). Antibody fragments can be made by various techniques, including but not limited to proteolytic digestion of an intact antibody as well as production by recombinant host cells (e.g., E. coli or phage), as described herein.

In other embodiments, a bispecific antibody of the present disclosure is a whole antibody. As defined herein, a whole antibody comprises two identical copies of a heavy (H) chain polypeptide and two identical copies of a light (L) chain polypeptide. Each of the heavy chains contains one N-terminal variable (VH) region and three C-terminal constant (C_{H1}, C_{H2}, and C_{H3}) regions, and each light chain contains one N-terminal variable (V_{L}) region and one C-terminal constant (C_{L}). The heavy chain C-terminal constant region contains the fragment crystallizable (Fc) domain, which determines antibody class and is responsible for humoral and cellular effector functions. Antibodies are divided into five major classes (or “isotypes”), IgG, IgM, IgA, IgD and IgE, which differ in their function in the immune system. IgGs are the most abundant immunoglobulins in the blood, representing 60% of total serum antibodies in humans. IgG antibodies may be subclassified as IgG1, IgG2, IgG3, and IgG4, named in order of their abundance in serum (IgG1 being the most abundant) (Vidarsson et al., Frontiers in Immunology. 5:520 (2014)). A whole bispecific antibody described herein (e.g., an anti-CFHR4/anti-C3 bispecific antibody, an anti-CFHR4/anti-VEGF bispecific antibody, and an anti-C3/anti-VEGF bispecific antibody) may be of any suitable class and/or subclass. In some embodiments, the antibody is of class IgG (e.g., IgG1, IgG2, IgG3, or IgG4). For example, the antibody may be an IgG1 antibody.

As discussed above, the Fc domain mediates several effector functions of antibodies, such as binding to receptors on target cells and complement fixation (triggering effector functions that eliminate the antigen). In some embodiments, the Fc domain may be modified or engineered to alter its effector functions. For example, Fc domains may be modified to improve antibody-dependent cellular cytotoxicity (ADCC) and antibody-dependent cellular phagocytosis (ADCP), and to control serum half-life. In some embodiments, the Fc domain of the anti-CFHR4 antibody may be engineered to modulate affinity for an Fc receptor, such as Fcγ receptors (FcγRs) and the neonatal Fc receptor (FcRn). Indeed, optimization of the interactions between antibodies and FcγRs has emerged as a promising approach for enhancing the activity of therapeutic antibodies for the treatment of various diseases (Mimoto et al., Curr. Pharm. Biotechnol. 17, 1298-1314 (2016); Lazar et al., Proc. Natl Acad. Sci. USA 103, 4005-4010 (2006); Richards et al., Mol. Cancer Ther. 7, 2517-2527 (2008); Nordstrom et al., Breast Cancer Res. 13, R123 (2011); and Kang, T. H., Jung, S. T., Exp Mol Med 51, 1-9 (2019)). The Fc domain also may be modified to improve serum half-life, e.g., by engineering IgG Fc for higher FcRn binding (Zalevsky et al., Nat. Biotechnol. 28, 157-159 (2010); and Dall'Acqua et al., J. Immunol. 169, 5171-5180 (2002)). In other embodiments, the Fc domain may be modified to create monovalency or antibody bispecificity for improving therapeutic potency. For example, an Fc domain may be generated that does not form a homodimer but remains as a soluble monomer, mFc, that exhibits high affinity for FcγRI but no detectable binding to FcγRIIIa. In other embodiments, a heterodimeric Fc domain may be generated to obtain bispecific properties for antigen binding to circumvent homodimer formation. Engineered Fc domains may be generated by inducing point mutations or by modifying glycosylation of the Fc domain (Saunders, K. O., Front Immunol. 2019; 10:1296; Kelley, R. F., Meng, Y. G., Liu et al., J Biol Chem. 2014; 289:3571-90; Monnet et al., MAbs. 2014; 6:422-36; Li et al., Proc Natl Acad Sci USA. 2017; 114:3485-90; and Lin et al., Proc Natl Acad Sci USA. 2015; 112:10611-6; Kang and Jung, supra).

As described above, the bispecific antibodies of the present disclosure (e.g., an anti-CFHR4/anti-C3 bispecific antibody, an anti-CFHR4/anti-VEGF bispecific antibody, and an anti-C3/anti-VEGF bispecific antibody) can be a monoclonal antibody, a human antibody, a humanized antibody, and/or a chimeric antibody. In some embodiments, the antibody is a fragment selected from the group consisting of Fab, Fab-C, Fab′-SH, Fv, scFv, and (Fab′)₂ fragments. In some embodiments, the bispecific antibodies of the present disclosure comprise two or more single-domain antibodies that form a bivalent antibody, a trivalent antibody, or a tetravalent antibody that recognizes different epitopes on the same or different antigens.

In some embodiments, the bispecific antibodies of the present disclosure (e.g., an anti-CFHR4/anti-C3 bispecific antibody, an anti-CFHR4/anti-VEGF bispecific antibody, and an anti-C3/anti-VEGF bispecific antibody) are chimeric antibodies. Certain chimeric antibodies are described, for example, in U.S. Pat. No. 4,816,567; and Morrison et al., Proc. Natl. Acad. Sci. USA. 81:6851-6855 (1984). In one example, a chimeric antibody comprises a non-human variable region (e.g., a variable domain derived from a mouse, rat, hamster, rabbit, or non-human primate, such as a monkey) and a human constant domain. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, for example, CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, for example, in Almagro and Fransson, Front. Biosci. 13:1619-1633 (2008), and are further described, for example, in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

In accordance with the above embodiments, the bispecific antibodies of the present disclosure (e.g., an anti-CFHR4/anti-C3 bispecific antibody, an anti-CFHR4/anti-VEGF bispecific antibody, and an anti-C3/anti-VEGF bispecific antibody) can be made into bivalent, trivalent, or tetravalent formats. For example, a bispecific antibody of the present disclosure can be a bivalent, bispecific antibody with heteromeric heavy chains (e.g., Triomab, knobs-into-holes (KIH), Duobody, etc). A bispecific antibody of the present disclosure can be a tetravalent multispecific antibody comprised of IgGs with other binding domains fused to either the N- or C-termini of either the heavy or light chains (e.g., dual variable domain [DVD], IgG-scFv fusion, Mabtyrin (IgG with non-antibody binding scaffold “centyrin” fused to C-terminal end of heavy chains). A bispecific antibody of the present disclosure can be comprised of IgGs to which additional antigen combining sites have been added within the structure (e.g., two-in-one antibodies, MAT “Modular Antibody Technology” platform from F-Star). A bispecific antibody of the present disclosure can be an engineered antibody fragment linked by short peptide linkers which can be made into bivalent, trivalent, or tetravalent formats addressing two to three targets (e.g., bispecific T-cell engager (BiTE), Nanobody platform, dual-affinity re-targeting (DART) antibodies, “tandem antibody” structures (TandAbs)). And a bispecific antibody of the present disclosure can be comprised of chemically coupled IgGs.

### Polypeptides and Expression Vectors

Embodiments of the present disclosure also include a polynucleotide encoding any of the bispecific antibodies of the present disclosure (e.g., an anti-CFHR4/anti-C3 bispecific antibody, an anti-CFHR4/anti-VEGF bispecific antibody, and an anti-C3/anti-VEGF bispecific antibody). In accordance with these embodiments, the present disclosure includes an expression vector comprising any of the polynucleotides encoding a bispecific antibody of the present disclosure. In some embodiments, the expression vector is suitable for manufacturing a bispecific antibody of the present disclosure for delivery of the antibody to a subject. In certain embodiments, the nucleic acid sequence is in the form of a vector. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 4th edition, Cold Spring Harbor Press, Cold Spring Harbor, N. Y. (2012), and Ausubel et al., Current Protocols in Molecular Biology, Greene Publishing Associates and John Wiley & Sons, New York, N.Y. (1994)).

In addition to the nucleic acid encoding an anti-CFHR4 antibody or antigen-binding fragment thereof, the vector desirably comprises expression control sequences, such as promoters, enhancers, polyadenylation signals, transcription terminators, internal ribosome entry sites (IRES), and the like, that provide for the expression of the antibody-encoding nucleic sequence in a host cell. Exemplary expression control sequences are known in the art and described in, for example, Goeddel, Gene Expression Technology: Methods in Enzymology, Vol. 185, Academic Press, San Diego, Calif. (1990).

A vector comprising a nucleic acid sequence encoding a bispecific antibody of the present disclosure (e.g., an anti-CFHR4/anti-C3 bispecific antibody, an anti-CFHR4/anti-VEGF bispecific antibody, and an anti-C3/anti-VEGF bispecific antibody), or an antigen-binding fragment thereof, may be introduced into a host cell that is capable of expressing the polypeptides encoded thereby, including any suitable prokaryotic or eukaryotic cell. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DH5a, DH10, MC1061 (ATCC No. 53338), and CC102). Suitable eukaryotic cells are known in the art and include, for example, yeast cells, insect cells, and mammalian cells. Examples of suitable yeast cells include those from the genera Hansenula, Kluyveromyces, Pichia, Rhinosporidium, Saccharomyces, and Schizosaccharomyces. Suitable insect cells include Sf-9 and HIS cells (Invitrogen, Carlsbad, Calif.) and are described in, for example, Kitts et al., Biotechniques, 14:810-817 (1993); Lucklow, Curr. Opin. Biotechnol., 4:564-572 (1993); and Lucklow et al., J. Virol., 67:4566-4579 (1993). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97:4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants also are suitable. Other suitable mammalian cell lines include, but are not limited to, mouse neuroblastoma N2A cells, HeLa, mouse L-929 cells, and BHK or Hak hamster cell lines, all of which are available from the ATCC. Methods for selecting suitable mammalian host cells and methods for transformation, culture, amplification, screening, and purification of such cells are well known in the art (see, e.g., Ausubel et al., eds., Short Protocols in Molecular Biology, 5th ed., John Wiley & Sons, Inc., Hoboken, N.J. (2002)). Preferably, the mammalian cell is a human cell.

In some embodiments, the vector can include means for attaching a detection moiety to a bispecific antibody of the present disclosure (e.g., an anti-CFHR4/anti-C3 bispecific antibody, an anti-CFHR4/anti-VEGF bispecific antibody, and an anti-C3/anti-VEGF bispecific antibody). In some embodiments, the vector can include means for attaching a purification moiety to a bispecific antibody of the present disclosure. Exemplary detection and/or purification moieties/tags that can be coupled to a bispecific antibody of the present disclosure includes, but is not limited to, hemagglutinin (HA), c-Myc, V5, DYKDDDDK, His tag (e.g., 6x-HIS), Glutathione S-Transferase (GST), Maltose Binding Protein (MBP), a fluorophore (e.g., Green Fluorescent Protein (GFP), Red Fluorescent Protein (RFP), mCherry, a chromophore, and/or a luminescent peptide (e.g., luciferase).

In some embodiments, the expression vector is suitable for use in gene therapy (e.g., an expression vector for delivering a polynucleotide encoding a bispecific antibody of the present disclosure to a subject). In some embodiments, the expression vector is a herpes simplex virus (HSV) vector, or a retrovirus vector. In some embodiments, the expression vector is an adeno-associated virus (AAV) vector, or comprises an AAV backbone. For example, AAV vectors have been designed, produced and used to mediate gene delivery in human subjects, including for therapeutic purposes. Typically, AAV vectors for use in gene transfer comprise a replication defective AAV genome lacking functional Rep and Cap coding viral sequences. Such replication defective AAV vectors more preferably lack most or all of the Rep and Cap coding sequences, and essentially retain one or two AAV ITR sequences and a packaging sequence. The defective genome is packaged in a viral particle, to form a defective, recombined AAV virus, also termed “AAV vector”. Methods of producing such AAV vectors have been disclosed in the literature, including using packaging cells, auxiliary viruses or plasmids, and/or baculovirus systems (Samulski et al., (1989) J. Virology 63, 3822; Xiao et al., (1998) J. Virology 72, 2224; Inoue et al., (1998) J. Virol. 72, 7024; WO98/22607; WO2005/072364). Methods of producing pseudotyped AAV vectors have also been reported (e.g., WO00/28004), as well as various modifications or formulations of AAV vectors, to reduce their immunogenicity upon in vivo administration (see e.g., WO01/23001; WOOO/73316; WO04/112727; WO05/005610; WO99/06562). AAV vectors may be prepared or derived from various serotypes of AAVs, which may be even mixed together or with other types of viruses to produce chimeric (e.g., pseudotyped) AAV viruses. Examples of tAAVs are human AAV4 vectors, human AAV7 vectors, human AAV9 vectors, human AAV10 vectors, or bovine AAV vectors. The AAV vector may be derived from a single AAV serotype or comprise sequences or components originating from at least two distinct AAV serotypes (pseudotyped AAV vector), e.g., an AAV vector comprising an AAV genome derived from one AAV serotype (for example AAV9), and a capsid derived at least in part from a distinct AAV serotype. An AAV vector, as used herein, is a vector which comprises at least one component part derivable from an adeno-associated virus. Preferably, that component part is involved in the biological mechanisms by which the vector infects or transduces target cells and expresses an anti-CFHR4 antibody of the present disclosure (e.g., ocular delivery/expression).

In other embodiments, the expression vector is a lentiviral vector (LV), or comprises an LV backbone. Lentiviruses are part of a larger group of retroviruses. A detailed list of lentiviruses may be found in Coffin et al (1997) “Retroviruses” Cold Spring Harbour Laboratory Press Eds: J M Coffin, SM Hughes, HE Varmus pp 758-763). For example, lentiviruses can be divided into primate and non-primate groups. Examples of primate lentiviruses include but are not limited to the human immunodeficiency virus (HIV), the causative agent of human auto immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), feline immunodeficiency virus (FIV), Maedi visna virus (MVV) and bovine immunodeficiency virus (BIV). In one embodiment, the lentiviral vector is derived from HIV-1, HIV-2, SIV, FIV, BIV, EIAV, CAEV or Visna lentivirus. The lentivirus family differs from retroviruses in that lentiviruses have the capability to infect both dividing and non-dividing cells (Lewis et al (1992) EM BO J 11 (8): 3053-3058 and Lewis and Emerman (1994) J Virol 68 (1): 510-516). In contrast, other retroviruses, such as MLV, are unable to infect non-dividing or slowly dividing cells such as those that make up, for example, muscle, brain, lung and liver tissue. A lentiviral vector, as used herein, is a vector which comprises at least one component part derivable from a lentivirus. Preferably, that component part is involved in the biological mechanisms by which the vector infects or transduces target cells and expresses an anti-CFHR4 antibody of the present disclosure (e.g., ocular delivery/expression).

Additional compositions and method for ocular gene therapy can be found in, e.g., Bordet, T., and Behar-Cohen, F., “Ocular gene therapies in clinical practice: viral vectors and nonviral alternatives,” Drug Discovery Today, Volume 24, Issue 8, August 2019, Pages 1685-1693). In some embodiments, gene therapy platforms, methods, and compositions that can be used to deliver a bispecific antibody of the present disclosure to a subject (e.g., ocular delivery) includes the platforms, methods, and compositions disclosed in US20220025396, US20220011308, US20210371877, US20210363192, US20190078099, US20190038724, and U.S. Pat. No. 10,494,646B2, which are incorporated herein by reference. In other embodiments, gene therapy platforms, methods, and compositions that can be used to deliver a bispecific antibody of the present disclosure to a subject (e.g., ocular delivery) includes the platforms, methods, and compositions based on HMR59 (Hemera Biosciences), which through its protein product soluble CD59, blocks the membrane attack complex that forms during the terminal step in the complement cascade. HMR59 is designed to be administered as a single intraocular injection.

In accordance with these embodiments, the present disclosure also provides a method of administering ocular gene therapy to a subject in need thereof comprising injecting a pharmaceutical composition comprising an effective amount of an expression vector described herein (e.g., an expression vector comprising a polynucleotides encoding a bispecific antibody of the present disclosure). As described further below, the present disclosure also provides a method of treating AMD (wet and/or dry AMD) and/or GA comprising administering a pharmaceutical composition comprising an effective amount of an expression vector described herein (e.g., an expression vector comprising a polynucleotides encoding a bispecific antibody of the present disclosure). In some embodiments, administering the pharmaceutical composition treats at least one AMD symptom (wet and/or dry AMD symptom) and/or at least one GA symptom.

### Methods of Making Bispecific Antibodies

In general, there are more than 100 different bispecific antibody formats that have been produced due to the modular architecture of antibodies. These formats vary in many ways, including based on their molecular weight, the number of antigen-binding sites, the spatial relationship between different binding sites, the valency for each antigen, the ability to support secondary immune functions, and the pharmacokinetic half-life. Recombinant bispecific antibodies can be divided into two classes: bispecific formats with Fc regions, and bispecific formats without Fc regions. Bispecific antibodies with an Fc region retain Fc-mediated effector functions, such as CDC and ADCC. These formats roughly include “knob into hole” IgG, crossMab, ortho-Fab IgG, DVD-Ig, two in one IgG, IgG-scFv and scFv2-Fc.

Bispecific antibodies with no Fc lack Fc-mediated effector functions. However, the smaller size of such antibodies offers a better tumor tissue penetration over IgG-like formats. In this format, the variable domains of each parental monoclonal antibody and the linkers are cloned and linked to form a single-chain bispecific antibody. These bispecific antibodies represent many formats, including tandem scFvs, diabody format, single-chain diabodies, tandem diabodies (TandAbs), dual-affinity retargeting molecules (DARTs), dock-and-lock (DNL), and nanobodies.

A number of strategies have been developed to generate bispecific antibodies. Hybrid hybridoma (also referred to as quadroma) was the earliest technology used to produce bispecific antibodies. It's based on the somatic fusion of two different hybridoma cell lines expressing murine IgGs of desired specificities. However, the real percentage of functional bispecific antibody by a quadroma cell line is unpredictable and a laborious process is required to isolate the bispecific antibody from the side products. By using molecular cloning technology, bispecific IgG antibodies can be assembled from two different heavy and light chains expressed in the same producer cell. The production of bispecific antibodies requires at least two plasmids for heterodimerized heavy chains and one plasmid for a common light chain or two light-chain plasmids if two different light chains are used. Notably, expressing HC and LC on separate plasmids can be advantageous because the manipulation of the plasmid ratio is an easy and efficient approach to optimize protein assembly for desired products. Subsequently, a laborious and time-consuming process is typically needed to select the most desirable clonal cell lines from a heterogeneous stable transfectant pool for large-scale antibody production. Generally, methods of making bispecific antibodies that can be divided into three groups: chemical recombination, cell fusion, and genetic manipulation (Reichert, J. M, and Dhimolca, E., Drug Discov Today, 2012 September; 17 (17-18): 954-63). Recent reviews on the different strategies in constructing bispecific antibodies, with therapeutic application emphasis, include Wu, C., Drug News Perspect. 2009 October; 22 (8): 453-8; Gu, J., and Ghayur, T., Methods Enzymol. 2012; 502:25-41; and Kontermann, R., MAbs, March-April 2012; 4 (2): 182-97.

The bispecific antibodies of the present disclosure can be made using any method known in the art. One approach for single cell manufacturing of bispecific antibodies is to use antibodies that are based on the same light chain in combination with a minimal set of Fc mutations to drive heavy chain heterodimerization. The resulting bispecific antibodies can readily be purified from the two undesired byproducts. As used herein the term “common light chain” refers to an immunoglobulin light chain comprising a variable domain that can productively associate with multiple heavy chain variable domains to form a paratope with each heavy chain variable region that is capable of specifically binding the epitope bound by the antibody in which the heavy chain variable region was originally encountered. Various antibody heavy or light chains, or portions thereof, are referred to herein as being “related” to the corresponding chain or portion thereof of an enumerated or specifically identified antibody. This denotes that the chain or portion thereof has identical sequence to the corresponding chain or portion thereof of an enumerated or specifically identified antibody, save for specifically indicated modifications, such as amino acid substitutions, of which there may be none. As described further herein, the bispecific antibodies of the present disclosure can be generated using this “common light chain” approach, including using one or more light chains of an anti-CHFR4 antibody

### Compositions and Methods of Treatment

The bispecific antibodies of the present disclosure (e.g., an anti-CFHR4/anti-C3 bispecific antibody, an anti-CFHR4/anti-VEGF bispecific antibody, and an anti-C3/anti-VEGF bispecific antibody) can be administered as part of a pharmaceutical composition in a therapeutically effective amount to treat an eye disease (e.g., AMD or GA). In some embodiments, the composition is suitable for ocular administration. In some embodiments, ocular administration comprises injection into vitreous fluid. In some embodiments, ocular administration comprises delivering the antibody using a conjunctival insert, a contact lens, a gel, a nanoparticle, a mucoadhesive polymer, an ointment, a solution, a suspension, eye drops, and/or an implant (e.g., Susvimo™). Recent methods and formulations for ocular administration can be found in, e.g., Souto, E. B., et al. “Advanced Formulation Approaches for Ocular Drug Delivery: State-Of-The-Art and Recent Patents,” Pharmaceutics, 2019 September; 11 (9): 460).

In accordance with these embodiments, the methods include administering a pharmaceutical composition comprising a therapeutically effective amount of a bispecific antibody of the present disclosure (e.g., an anti-CFHR4/anti-C3 bispecific antibody, an anti-CFHR4/anti-VEGF bispecific antibody, and an anti-C3/anti-VEGF bispecific antibody). In some embodiments, the pharmaceutical composition is administered ocularly and treats at least one AMD symptom. In some embodiments, AMD comprises wet AMD. In some embodiments, AMD comprises dry AMD (e.g., GA). In some embodiments, the at least one AMD symptom comprises visual distortion, reduced central vision, blurred vision, and/or difficulty adapting to low light. In some embodiments, administering the pharmaceutical composition reduces complement activation in the subject's eye.

As used herein, the terms “treatment,” “treating,” and the like refer to obtaining a desired pharmacologic and/or physiologic effect. In some embodiments, the effect is therapeutic, i.e., the effect partially or completely cures a disease and/or adverse symptom attributable to the disease. To this end, the methods of the present disclosure comprise administering a “therapeutically effective amount” of a bispecific antibody, or composition comprising a bispecific antibody. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the monoclonal antibody to elicit a desired response in the individual. For example, a therapeutically effective amount of a bispecific antibody of the present disclosure is an amount which treats at least one AMD and/or GA symptom in a subject. In some embodiments, the pharmacologic and/or physiologic effect may be prophylactic, i.e., the effect completely or partially prevents a disease or symptom thereof. In this respect, the methods of the present disclosure comprise administering a “prophylactically effective amount” of a bispecific antibody or composition comprising a bispecific antibody. A “prophylactically effective amount” refers to an amount effective, at dosages and for periods of time necessary, to achieve a desired prophylactic result (e.g., prevention of AMD and/or GA onset).

A typical dose of a therapeutically effective amount of a bispecific antibody of the present disclosure (e.g., an anti-CFHR4/anti-C3 bispecific antibody, an anti-CFHR4/anti-VEGF bispecific antibody, and an anti-C3/anti-VEGF bispecific antibody) can range from, for example, about 0.0001 mg/dose to about 100 mg/dose for each eye to be treated. In some embodiments, a therapeutically effective amount of a bispecific antibody of the present disclosure can range from about 0.001 mg/dose to about 100 mg/dose, from about 0.01 mg/dose to about 100 mg/dose, from about 0.05 mg/dose to about 50 mg/dose, from about 0.1 mg/dose to about 10 mg/dose, from about 0.5 mg/dose to about 5 mg/dose, and from about 1 mg/dose to about 10 mg/dose. In some embodiments, a therapeutically effective concentration of a bispecific antibody of the present disclosure can range from, for example, about 0.0001 mg to about 100 mg of the antibody per milliliter of solution. In some embodiments, a therapeutically effective concentration of a bispecific antibody of the present disclosure can range from about 0.001 mg/ml to about 100 mg/ml, from about 0.01 mg/ml to about 100 mg/ml, from about 0.1 mg/ml to about 100 mg/ml, from about 1.0 mg/ml and about 100 mg/ml, from about 0.001 mg/ml and about 50 mg/ml, from about 0.01 mg/ml and about 50 mg/ml, from about 0.1 mg/ml and about 50 mg/ml, from about 0.1 mg/ml and about 25 mg/ml, from about 0.1 mg/ml and about 10 mg/ml, and from about 1.0 mg/ml and about 10 mg/ml. In some embodiments, a therapeutically effective dose of a bispecific antibody of the present disclosure can be, exactly or approximately, 0.1 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 2.0 mg, 3.0 mg, 4.0 mg, 5.0 mg, 10.0 mg, 15.0 mg, 20.0 mg, or 25.0 mg, or can fall within a range delimited by any two of the foregoing values. For example, in certain embodiments, a sustained release formulation, (e.g., an ocular implant) can be, exactly or approximately, 0.1 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 2.0 mg, 3.0 mg, 4.0 mg, 5.0 mg, 10.0 mg, 15.0 mg, 20.0 mg, or 25.0 mg of a bispecific antibody, or an amount that falls within a range delimited by any two of the foregoing values.

Therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the present disclosure. The desired dosage can be delivered by a single bolus administration of the composition, by multiple bolus administrations of the composition, or by continuous infusion administration of the composition. The composition comprising a bispecific antibody, or antigen-binding fragment thereof, can be administered to a mammal using standard administration techniques, including ocular, oral, intravenous, intraperitoneal, subcutaneous, pulmonary, transdermal, intramuscular, intranasal, buccal, sublingual, or suppository administration. The composition preferably is suitable for ocular administration.

In accordance with the compositions and methods of treatment described herein, embodiments of the present disclosure include bispecific antibodies that have enhanced half-life (e.g., after ocular administration), such that the bispecific antibody can be administered less often to a subject. In some embodiments, the antibody comprises a half-life extension moiety. In some embodiments, the half-life extension moiety comprises a polypeptide that can be coupled to a bispecific antibody of the present disclosure by any means known in the art (e.g., generation of a fusion protein). In some embodiments, the polypeptide that can be coupled to a bispecific antibody of the present disclosure is at least 20 amino acids in length and comprises any combination of G, A, S T, E, and P residues. In some embodiments, the half-life extension polypeptide is attached to the C-terminus or N-terminus of the antibody. In some embodiments, this is referred to as “XTENylation,” as described further in U.S. Pat. Nos. 8,933,197, 7,846,445, 7,855,279, 8,492,530, 9,938,331, 8,673,860, 9,371,369, 9,926,351, 10,961,287, 10,172,953, and 10,953,073.

The present disclosure also provides a composition comprising any of the bispecific antibodies or antigen-binding fragments thereof described herein (e.g., an anti-CFHR4/anti-C3 bispecific antibody, an anti-CFHR4/anti-VEGF bispecific antibody, and an anti-C3/anti-VEGF bispecific antibody). The composition desirably is a pharmaceutically acceptable (e.g., physiologically acceptable) composition, which comprises a carrier, preferably a pharmaceutically acceptable (e.g., physiologically acceptable) carrier, and the bispecific antibody or antigen-binding fragment thereof. Any suitable carrier can be used within the context of the present disclosure, and such carriers are well known in the art. For example, the composition may contain preservatives, such as, for example, methylparaben, propylparaben, sodium benzoate, and benzalkonium chloride. A mixture of two or more preservatives optionally may be used. In addition, buffering agents may be included in the composition. Suitable buffering agents include, for example, citric acid, sodium citrate, phosphoric acid, potassium phosphate, and various other acids and salts. A mixture of two or more buffering agents optionally may be used. Methods for preparing compositions for pharmaceutical use are known to those skilled in the art and are described in, for example, Remington: The Science and Practice of Pharmacy, Lippincott Williams & Wilkins; 21st ed. (May 1, 2005).

Once administered to a mammal (e.g., a human), the biological activity of the bispecific antibody, or antigen-binding fragment thereof, can be measured by any suitable method known in the art. For example, the biological activity can be assessed by determining the stability of the bispecific antibody. The biological activity of the bispecific antibody also can be assessed by determining its binding affinity to antigen-containing peptides and/or by assessing its binding affinity to peptides with which it may cross-react. The term “affinity” refers to the equilibrium constant for the reversible binding of two agents and is expressed as the dissociation constant (K_{D}). Affinity of a binding agent to a ligand, such as affinity of an antibody for an epitope, can be, for example, from about 1 femtomolar (fM) to about 1 millimolar (mM) (e.g., from about 1 picomolar (pM) to about 1 nanomolar (nM), or from about 1 nM to about 1 micromolar (μM)). In some embodiments, the affinity of an anti-CFHR4 antibody may be from about 1 nm to about 20 nm, and desirably from about 5 nm to about 10 nm. Antibody affinity for an antigen or epitope of interest can be measured using any art-recognized assay. Such methods include, for example, fluorescence activated cell sorting (FACS), separable beads (e.g., magnetic beads), antigen panning, and/or ELISA (see, e.g., Janeway et al. (eds.), Immunobiology, 5th ed., Garland Publishing, New York, N.Y., 2001).

In some embodiments, a bispecific antibody, or composition comprising a bispecific antibody, may be administered alone or in combination with other drugs. For example, the bispecific antibodies of the present disclosure (e.g., an anti-CFHR4/anti-C3 bispecific antibody, an anti-CFHR4/anti-VEGF bispecific antibody, and an anti-C3/anti-VEGF bispecific antibody) can be administered in combination with other agents for the treatment or prevention of AMD and/or GA, as disclosed herein. For example, bispecific antibodies of the present disclosure, or antibody conjugates, fusion proteins, or polymeric formulations thereof, can be used either alone or in combination with other agents in a therapy. For instance, a bispecific antibody may be co-administered with at least one additional therapeutic agent. In certain embodiments, an additional therapeutic agent is another antibody, a chemotherapeutic agent, a cytotoxic agent, an anti-angiogenic agent, an immunosuppressive agent, a prodrug, a cytokine, a cytokine antagonist, cytotoxic radiotherapy, a corticosteroid, an anti-emetic, a cancer vaccine, an analgesic, a growth-inhibitory agent, or combinations thereof.

For example, in certain embodiments, any of the preceding methods further comprises administering one or more additional compounds. In certain embodiments, the bispecific antibody, antibody conjugate, fusion protein, or polymeric formulation is administered simultaneously with the additional compound(s). In certain embodiments, the bispecific antibody, antibody conjugate, fusion protein, or polymeric formulation is administered before or after the additional compound(s). In certain embodiments, the additional compound binds to a second biological molecule selected from the group consisting of VEGF, IL-1B; IL-6; IL-6R; IL-13; IL-13R; PDGF; angiopoietin; Ang2; Tie2; SIP; integrins αvβ3, αvβ5, and α5β1; betacellulin; apelin/APJ; erythropoietin; complement factor D; TNFα; HtrA1; a VEGF receptor; ST-2 receptor; and proteins genetically linked to AMD risk, such as complement pathway components C2, factor B, factor H, CFHR3, C3b, C5, C5a, and C3a; HtrA1; ARMS2; TIMP3; HLA; interleukin-8 (IL-8); CX3CR1; TLR3; TLR4; CETP; LIPC; COL10A1; and TNERSF10A. In some embodiments, the additional compound is an antibody or antigen-binding fragment thereof. In some embodiments according to (or as applied to) any of the embodiments above, the ocular disorder is an intraocular neovascular disease selected from the group consisting of proliferative retinopathies, choroidal neovascularization (CNV), age-related macular degeneration (AMD), geographic atrophy (GA), diabetic and other ischemia-related retinopathies, diabetic macular edema, pathological myopia, von Hippel-Lindau disease, histoplasmosis of the eye, retinal vein occlusion (RVO), including CRVO and BRVO, corneal neovascularization, retinal neovascularization, and retinopathy of prematurity (ROP).

In some instances, a bispecific antibody of the present disclosure, or an antibody conjugate, fusion protein, and/or polymeric formulation thereof, may be administered in combination with at least one additional therapeutic agent for treatment of an ocular disorder, for example, an ocular disorder described herein (e.g., AMD (e.g., wet AMD or dry AMD), GA, DME, DR, or RVO). Exemplary additional therapeutic agents for combination therapy for treatment of ocular disorders include, without limitation, anti-angiogenic agents, such as VEGF antagonists, including, for example, anti-VEGF antibodies (e.g., the anti-VEGF Fab LUCENTIS® (ranibizumab)), soluble receptor fusion proteins (e.g., the recombinant soluble receptor fusion protein EYLEA® (aflibercept, also known as VEGF Trap Eye; Regeneron/Aventis)), aptamers (e.g., the anti-VEGF pegylated aptamer MACUGEN® (pegaptanib sodium; NeXstar Pharmaceuticals/OSI Pharmaceuticals)), and VEGFR tyrosine kinase inhibitors (e.g., 4-(4-bromo-2-fluoroanilino)-6-methoxy-7-(1-methylpiperidin-4-ylmethoxy) quinazoline (ZD6474), 4-(4-fluoro-2-methylindol-5-yloxy)-6-methoxy-7˜ (3-pyrrolidin-1-ylpropoxy) quinazoline (AZD2171), vatalanib (PTK787), semaxaminib (SU5416; SUGEN), and SUTENT® (sunitinib)); Tryptophanyl-tRNA synthetase (TrpRS); squalamine; RETAANE® (anecortave acetate for depot suspension; Alcon, Inc.); Combretastatin A4 Prodrug (CA4P); MIFEPREX® (mifepristone-ru486); subtenon triamcinolone acetonide; intravitreal crystalline triamcinolone acetonide; matrix metalloproteinase inhibitors (e.g., Prinomastat (AG3340; Pfizer)); fluocinolone acetonide (including fluocinolone intraocular implant; Bausch & Lomb/Control Delivery Systems); linomide; inhibitors of integrin B3 function; angiostatin, and combinations thereof.

Further examples of additional therapeutic agents that can be used in combination with a bispecific antibody of the present disclosure, or an antibody conjugate, fusion protein, and/or polymeric formulation thereof, for treatment of an ocular disorder (e.g., AMD, GA, DME, DR, or RVO), include, but are not limited to, VISUDYNE® (verteporfin; a light-activated drug that is typically used in conjunction with photodynamic therapy with a non-thermal laser), PKC412, Endovion (NS 3728; NeuroSearch A/S), neurotrophic factors (e.g., glial derived neurotrophic factor (GDNF) and ciliary neurotrophic factor (CNTF)), diltiazem, dorzolamide, PHOTOTROP®, 9-cis-retinal, eye medication (e.g., phospholine iodide, echothiophate, or carbonic anhydrase inhibitors), veovastat (AE-941; AEterna Laboratories, Inc.), Sirna-027 (AGF-745; Sirna Therapeutics, Inc.), neurotrophins (including, by way of example only, NT-4/5, Genentech), Cand5 (Acuity Pharmaceuticals), INS-37217 (Inspire Pharmaceuticals), integrin antagonists (including those from Jerini AG and Abbott Laboratories), EG-3306 (Ark Therapeutics Ltd.), BDM-E (BioDiem Ltd.), thalidomide (as used, for example, by EntreMed, Inc.), cardiotrophin-1 (Genentech), 2-methoxyestradiol (Allergan/Oculex), DL-8234 (Toray Industries), NTC-200 (Neurotech), tetrathiomolybdate (University of Michigan), LYN-002 (Lynkeus Biotech), microalgal compound (Aquasearch/Albany, Mera Pharmaceuticals), D-9120 (Celitech Group plc), ATX-S10 (Hamamatsu Photonics), TGF-beta 2 (Genzyme/Celtrix), tyrosine kinase inhibitors (e.g., those from Allergan, SUGEN, or Pfizer), NX-278-L (NeXstar Pharmaceuticals/Gilead Sciences), Opt-24 (OPTIS France SA), retinal cell ganglion neuroprotectants (Cogent Neurosciences), N-nitropyrazole derivatives (Texas A&M University System), KP-102 (Krenitsky Pharmaceuticals), cyclosporin A, therapeutic agents used in photodynamic therapy (e.g., VISUDYNE®; receptor-targeted PDT, Bristol-Myers Squibb, Co.; porfimer sodium for injection with PDT; verteporfin, QLT Inc.; rostaporfin with PDT, Miravent Medical Technologies; talaporfin sodium with PDT, Nippon Petroleum; and motexafin lutetium, Pharmacyclics, Inc.), antisense oligonucleotides (including, by way of example, products tested by Novagali Pharma SA and ISIS-13650, Isis Pharmaceuticals), and combinations thereof.

A bispecific antibody of the present disclosure, or an antibody conjugate, fusion protein, and/or polymeric formulation thereof, may be administered in combination with a therapy or surgical procedure for treatment of an ocular disorder (e.g., AMD, GA DME, DR, or RVO), including, for example, laser photocoagulation (e.g., panretinal photocoagulation (PRP)), drusen lasering, macular hole surgery, macular translocation surgery, implantable miniature telescopes, PHI-motion angiography (also known as micro-laser therapy and feeder vessel treatment), proton beam therapy, microstimulation therapy, retinal detachment and vitreous surgery, scleral buckle, submacular surgery, transpupillary thermotherapy, photosystem I therapy, use of RNA interference (RNAi), extracorporeal rheopheresis (also known as membrane differential filtration and rheotherapy), microchip implantation, stem cell therapy, gene replacement therapy, ribozyme gene therapy (including gene therapy for hypoxia response element, Oxford Biomedica; Lentipak, Genetix; and PDEF gene therapy, Gen Vec), photoreceptor/retinal cells transplantation (including transplantable retinal epithelial cells, Diacrin, Inc.; retinal cell transplant, Cell Genesys, Inc.), acupuncture, and combinations thereof.

In some embodiments, a bispecific antibody of the present disclosure, or an antibody conjugate, fusion protein, and/or polymeric formulation thereof, can be administered in combination with an anti-angiogenic agent for treatment of an ocular disorder (e.g., AMD, GA, DME, DR, or RVO). Any suitable anti-angiogenic agent can be used in combination with an antibody of the present disclosure, including, but not limited to, those listed by Carmeliet et al. Nature 407:249-257, 2000. In some embodiments, the anti-angiogenic agent is a VEGF antagonist, including, but not limited to, an anti-VEGF antibody (e.g., the anti-VEGF Fab LUCENTIS® (ranibizumab), RTH-258 (formerly ESBA-1008, an anti-VEGF single-chain antibody fragment; Novartis), or a bispecific anti-VEGF antibody (e.g., an anti-VEGF/anti-angiopoeitin 2 bispecific antibody such as RG-7716; Roche)), a soluble recombinant receptor fusion protein (e.g., EYLEA® (aflibercept)), a VEGF variant, a soluble VEGFR fragment, an aptamer capable of blocking VEGF (e.g., pegaptanib) or VEGFR, a neutralizing anti-VEGFR antibody, a small molecule inhibitor of VEGFR tyrosine kinases, an anti-VEGF DARPin® (e.g., abicipar pegol), a small interfering RNAs which inhibits expression of VEGF or VEGFR, a VEGER tyrosine kinase inhibitor (e.g., 4˜ (4-bromo-2-fluoroanilino)-6-methoxy-7-(1-methylpiperidin-4-ylmethoxy) quinazoline (ZD6474), 4-(4-fluoro-2-methylindol-5-yloxy)-6-methoxy-7-(3-pyrrolidin-1-ylpropoxy) quinazoline (AZD2171), vatalanib (PTK787), semaxaminib (SUS416; SUGEN), and SUTENT® (sunitinib)), and combinations thereof.

Other suitable anti-angiogenic agents that may be administered in combination with a bispecific antibody of the present disclosure, or an antibody conjugate, fusion protein, and/or polymeric formulation thereof, for treatment of an ocular disorder (e.g., AMD, GA, DME, DR, or RVO) include corticosteroids, angiostatic steroids, anecortave acetate, angiostatin, endostatin, tyrosine kinase inhibitors, matrix metalloproteinase (MMP) inhibitors, insulin-like growth factor-binding protein 3 (IGFBP3), stromal derived factor (SDF-1) antagonists (e.g., anti-SDF-1 antibodies), pigment epithelium-derived factor (PEDF), gamma-secretase, Delta-like ligand 4, integrin antagonists, hypoxia-inducible factor (HIF)-1α antagonists, protein kinase CK2 antagonists, agents that inhibit stem cell (e.g., endothelial progenitor cell) homing to the site of neovascularization (e.g., an anti-vascular endothelial cadherin (CD-144) antibody and/or an anti-SDF-1 antibody), and combinations thereof.

In some embodiments, a bispecific antibody of the present disclosure, or an antibody conjugate, fusion protein, and/or polymeric formulation thereof, can be administered in combination with an agent that has activity against neovascularization for treatment of an ocular disorder (e.g., AMD, GA, DME, DR, or RVO), such as an anti-inflammatory drug, a mammalian target of rapamycin (mTOR) inhibitor (e.g., rapamycin, AFINITOR® (everolimus), and TORISEL® (temsirolimus)), cyclosporine, a tumor necrosis factor (TNF) antagonist (e.g., an anti-TNFα antibody or antigen-binding fragment thereof (e.g., infliximab, adalimumab, certolizumab pegol, and golimumab) or a soluble receptor fusion protein (e.g., etanercept)), an anti-complement agent, a nonsteroidal anti-inflammatory agent (NSAID), or combinations thereof.

Any suitable AMD therapeutic agent can be administered as an additional therapeutic agent in combination with a bispecific antibody of the present disclosure, or an antibody conjugate, fusion protein, and/or polymeric formulation thereof, for treatment of an ocular disorder (e.g., AMD, GA, DME, DR, or RVO), including, but not limited to, a VEGF antagonist, for example, an anti-VEGF antibody (e.g., LUCENTIS® (ranibizumab), RTH-258 (formerly ESBA-1008, an anti-VEGF single-chain antibody fragment; Novartis), or a bispecific anti-VEGF antibody (e.g., an anti-VEGF/anti-angiopoeitin 2 bispecific antibody such as RG-7716; Roche)), a soluble VEGF receptor fusion protein (e.g., EYLEA® (aflibercept)), an anti-VEGF DARPin® (e.g., abicipar pegol; Molecular Partners AG/Allergan), or an anti-VEGF aptamer (e.g., MACUGEN®) (pegaptanib sodium)); a platelet-derived growth factor (PDGF) antagonist, for example, an anti-PDGF antibody, an anti-PDGFR antibody (e.g., REGN2176-3), an anti-PDGF-BB pegylated aptamer (e.g., FOVISTA®; Ophthotech/Novartis), a soluble PDGFR receptor fusion protein, or a dual PDGF/VEGF antagonist (e.g., a small molecule inhibitor (e.g., DE-120 (Santen) or X-82 (TyrogeneX)) or a bispecific anti-PDGF/anti-VEGF antibody)); VISUDYNE® (verteporfin) in combination with photodynamic therapy; an antioxidant; a complement system antagonist, for example, a complement factor C5 antagonist (e.g., a small molecule inhibitor (e.g., ARC-1905; Opthotech) or an anti-C5 antibody (e.g., LFG-316; Novartis), a properdin antagonist (e.g., an anti-properdin antibody, e.g., CLG-561; Alcon), or a complement factor D antagonist (e.g., an anti-complement factor D antibody, e.g., lampalizumab; Roche)); a visual cycle modifier (e.g., emixustat hydrochloride); squalamine (e.g., OHR-102; Ohr Pharmaceutical); vitamin and mineral supplements (e.g., those described in the Age-Related Eye Disease Study 1 (AREDS1; zinc and/or antioxidants) and Study 2 (AREDS2; zinc, antioxidants, lutein, zeaxanthin, and/or omega-3 fatty acids)); a cell-based therapy, for example, NT-501 (Renexus); PH-05206388 (Pfizer), huCNS-SC cell transplantation (StemCells), CNTO-2476 (Janssen), OpRegen (Cell Cure Neurosciences), or MA09-hRPE cell transplantation (Ocata Therapeutics); a tissue factor antagonist (e.g., hl-con1; Iconic Therapeutics); an alpha-adrenergic receptor agonist (e.g., brimonidine tartrate); a peptide vaccine (e.g., S-646240; Shionogi); an amyloid beta antagonist (e.g., an anti-beta amyloid monoclonal antibody, e.g., GSK-933776); an SIP antagonist (e.g., an anti-SIP antibody, e.g., iSONEP™; Lpath Inc); a ROBO4 antagonist (e.g., an anti-ROBO4 antibody, e.g., DS-7080a; Daiichi Sankyo); a lentiviral vector expressing endostatin and angiostatin (e.g., RetinoStat); and any combination thereof. In some instances, AMD therapeutic agents (including any of the preceding AMD therapeutic agents) can be co-formulated. For example, the anti-PDGFR antibody REGN2176-3 can be co-formulated with aflibercept (EYLEA®). In some instances, such a co-formulation can be administered in combination with an antibody of the present disclosure. In some instances, the ocular disorder is AMD (e.g., wet AMD).

In addition to therapeutic uses, a bispecific antibody or antigen-binding fragment, described herein can be used in diagnostic or research applications. Research applications include, for example, methods that utilize the bispecific antibody and a label to detect the corresponding antigen(s) in a sample, e.g., in a human body fluid or in a cell or tissue extract. The bispecific antibody or antigen-binding fragment thereof may be employed in any suitable assay for measuring a corresponding antigen(s) in a sample for diagnostic and/or research purposes. Such assays include, but are not limited to, sandwich immunoassays, enzyme immunoassays (EIA), enzyme-linked immunosorbent assays (ELISA), lateral flow assays, competitive inhibition immunoassays (e.g., forward and reverse), competitive binding assays, Forster resonance energy transfer (FRET), one-step antibody detection assays, single molecule detection assays, radioimmunoassays (RIA), and FACS. Such methods are disclosed in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792; and Adamczyk et al., Anal. Chim. Acta, 579 (1): 61-67 (2006).

The bispecific antibody or antigen-binding fragment thereof can be provided in a kit, e.g., a packaged combination of reagents in predetermined amounts with instructions for performing an assay using the antibody (e.g., an assay that detects a corresponding antigen(s)). As such, the disclosure provides a kit comprising the antibody or antigen-binding fragment described herein and instructions for use thereof. The instructions can be in paper form or computer-readable form, such as a disk, CD, DVD, etc. Alternatively or additionally, the kit can comprise a calibrator or control, and/or at least one container (e.g., tube, microtiter plates, or strips) for conducting an assay, and/or a buffer, such as an assay buffer or a wash buffer. Ideally, the kit comprises all components, i.e., reagents, standards, buffers, diluents, etc., which are necessary to perform the assay. Other additives may be included in the kit, such as stabilizers, buffers (e.g., a blocking buffer or lysis buffer), and the like. The relative amounts of the various reagents can be varied to provide for concentrations in solution of the reagents which substantially optimize the sensitivity of the assay. The reagents may be provided as dry powders (typically lyophilized), including excipients which on dissolution will provide a reagent solution having the appropriate concentration.

The following examples further illustrate the various embodiments of the present disclosure but should not be construed as in any way limiting its scope.

### EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Genetic Analysis of CFHR4 in Geographic Atrophy. This example describes analysis that was performed to determine the effects of CFHR1/4 deletions on Age-Related Macular Degeneration (AMD) in FinnGen. There are three common haplotypes composed of a variant in CFH (rs1061170 Y402H) and a variant that is an eQTL and pQTL for CFH and CFHR4 (rs10922109) that are associated with three different levels of risk. The high AMD risk haplotype (H402+high CFHR4 expression, rs106117_C (ref)/rs10922109_C (ref)) was set as the “reference” haplotype to allow comparison to other haplotypes (frequency in cases: 0.56, frequency in controls: 0.36). The haplotype carrying Y402 (the alternate allele at rs106117) and with High CFHR4/low CFH expression (reference allele at rs10922109) is associated with “medium” AMD risk (frequency in cases: 0.196, frequency in controls: 0.21). The “low” risk haplotype carries Y402 (the alternate allele at rs106117) and has low CFHR4/high CFH expression (alternate allele at rs10922109), with a frequency in cases: 0.22, frequency in controls: 0.43). It was hypothesized that haplotypes that carry a CFHR1/4 deletion will have a different risk for AMD on the medium risk background.

Genetic variation at this locus provides a natural experiment to determine the effect of CFHR1/4 deletion on AMD. The CFHR1/4 deletion is not directly genotyped in FinnGen but there are variants (rs528922402 and rs188297593) that imperfectly tag (r2=0.36, D′=1 in Finland) non-overlapping subsets of the deletion in Finnish populations in 1000Genomes dataset (HapMap phase3). The variant rs188297593 has a D′<1.0 in HapMap Phase 3 samples of European ancestry (1 of 4 carriers are not tagging CFHR1/4 deletion). These two variants capture a subset but not all carriers of the deletion.

To determine the effect of the CFH variant (rs1061170), CFHR4/CFH eQTL/pQTL (rs10922109) and CFHR1/4 deletion (tagged by rs528922402 and rs188297593), AMD risk was examined for individuals from FinnGen R6. Haplotypes composed of the individual variants were generated and a logistic regression was run in R to predict the effect of the haplotype on AMD risk adjusting for sex, age, and PC's. The reference haplotype was used as reference such that all other haplotype effects are in relation to that one.

Consistent with the hypothesis that CFHR4 contributes directly to AMD risk, “medium” risk haplotypes with CFHR4 deleted conferred increased protection from AMD relative to “medium” risk haplotypes with intact CFHR4.

TABLE 1
Results from haplotype analysis in FinnGen showing AMD risk from each
haplotype with Odds Ratio and p-values from logistic regression.

| | | rs528922402 | rs188297593 | | | | | | |
|---|---|---|---|---|---|---|---|---|---|
| | | (GAA/G) | (T/A) | | | | | | |
| rs1061170 | rs10922109 | CFHR1/4 | CFHR1/4 | | | | | | |
| (C/T) | (C/A) | deletion | deletion | | | | | | |
| CFH risk | CFHR4 / | tagging | tagging | AMD | | Frequency | Frequency | Odds | P- |
| variant | CFH pQTL | variant | variant | cases | Controls | in Cases | in Controls | Ratio | value |
| H402 (C) | High | — | — | 5,447 | 218,864 | 0.5622 | 0.4336 | 1 (Ref) | — |
| | CFHR4/ low | | | | | | | | |
| | CFH (C) | | | | | | | | |
| Y402 (T) | High | — | — | 1,452 | 75,112 | 0.1499 | 0.1488 | 0.75 | 6e−19 |
| | CFHR4/ low | | | | | | | [0.71, | |
| | CFH (C) | | | | | | | 0.80] | |
| Y402 (T) | High | — | CFHR1/4 | 100 | 6,188 | 0.0103 | 0.0123 | 0.65 | 6e−05 |
| | CFHR4/ low | | deletion | | | | | [0.53, | |
| | CFH (C) | | | | | | | 0.80] | |
| Y402 (T) | High | CFHR1/4 | — | 43 | 4,011 | 0.0044 | 0.0079 | 0.42 | 5e−08 |
| | CFHR4/ low | deletion | | | | | | [0.31, | |
| | CFH (C) | | | | | | | 0.57] | |
| Y402 (T) | Low | — | — | 2,635 | 200,601 | 0.2720 | 0.3974 | 0.50 | 2e−163 |
| | CFHR4/ high | | | | | | | [0.48, | |
| | CFH (A) | | | | | | | 0.53] | |

### Example 2

Generation of recombinant CFHR4 proteins. Recombinant protein preparations were undertaken to produce specific proteins which could be used as immunogens/antigens, screening reagents, and/or control reagents. All efforts were made in service of the goal of generating a panel of anti-CFHR4b antibodies with the desired properties.

Proteins with the sequence corresponding to human CFHR4b, human CFHL-1 wild type, human CFHL-1 with Tyrosine 402 mutated to Histidine, and cynomolgus CFHR4b were successfully produced with various protein tags. When finished, all protein preparations exceeded 90% purity by analytical methods and were less than <1 endotoxin unit per milliliter. Examples of final specifications for individual preparations are below.

Beginning with in silico analysis, appropriate wild type amino acid sequences of human and cynomolgus CFHR4b and human CFHL-1 were located and extracted from publicly available databases. The sequences were then further analyzed and modified manually using Geneious Prime software in accordance with the project plan. Sequences were examined for liabilities and modified to add further amino acid sequence which encoded for “tags” to facilitate purification, lower immunogenicity, or simplify analytical screening. Examples of protein tags used in this work include the HIS tag, a proprietary mouse IgG Fc tag, and the Avi tag. Tags were separated from each other and from the CFHR4b or CFHL-1 sequence with short linker sequences. All tags were added to the C-terminal end of the sequences. Additionally, the native signal peptide was removed and replaced with a signal peptide conducive to recombinant expression.

After all modifications were complete, the amino acid sequences were back translated into DNA sequences and optimized for the codon biases found in the human genome. These optimized DNA sequences were sent to Integrated DNA Technologies and produced as DNA fragment(s) with DNA overhangs added to the 5′ and 3′ ends. Utilizing the DNA overhangs and the Gibson cloning method, these DNA fragments were assembled into the expected sequence and cloned into a mammalian expression plasmid driven by a CMV promoter. The plasmid was propagated in E. coli with appropriate antibiotic selection and prepared, at a scale useful for recombinant expression, utilizing commercially available preparation kits purchased from Qiagen. The sequence of the plasmid and the expression gene was then confirmed using Sanger sequencing.

Using polyethylenimine, the sequence verified plasmid was transfected into human embryonic kidney cells adapted for recombinant expression. One day after transfection, the cells were supplemented with chemicals and nutrients designed to increase recombinant protein expression. These supplements include sodium propionate, valproic acid, glucose, glutamine, and a variety of yeast lysates. Five days after transfection, the expressing cell cultures were harvested. As the recombinant protein is secreted into the growth medium, the cells, cell fragments, and cell debris, were removed via centrifugation and filtration through a membrane with pores no greater than 0.22 microns. The clarified culture medium, conditioned with the recombinant protein, was now ready for purification.

Using a FPLC (Fast pressure liquid chromatography) and the appropriate commercially available pre-packed affinity chromatography column for the C-terminal tag(s) the protein of interest was purified from the cell culture medium and immobilized on the chromatography column. For HIS tagged proteins, a Ni-NTA agarose column was used. For Fc tagged proteins, a proA agarose column was employed. Multiple column washes followed, each specific for the type of column/tag/chromatography. The protein of interest was eluted from the column with 300 mM of imidazole for HIS tagged proteins, and with 100 mM Citrate pH 3.5 for Fc tagged proteins. Following elution, the Fc tagged proteins were adjusted to a neutral pH using IM Hepes pH 9. Neutralization was unnecessary for HIS tagged proteins, as the elution buffer is at a neutral pH.

Protein quality and quantity were assessed using a combination of SDS-PAGE gel electrophoresis, spectrophotometry, and analytical-SEC (size exclusion chromatography). Most proteins for this project required further purification. To further polish the proteins, the FPLC and a second chromatography column were employed. Separation by size, accomplished with a size exclusion column, enabled increased protein purities up to >90%. SEC also enabled buffer exchange out of the affinity chromatography elution buffer and into the final buffer of choice (PBS). The protein sample then moved onto quality control. If, at any point during purification, protein concentration needed to be increased, Amicon Ultra molecular weight cutoff (MWCO) filtration units were used to concentrate protein by separating protein from buffer. MWCO was chosen to ensure compatibility with the size of the protein of interest. Before moving to final quality control, all final samples were concentrated to >1 milligram per milliliter.

Endotoxin contamination in the final protein sample was assayed using Charles River's Endosafe PTS system. Spectrophotometry determined the final protein concentration. Three micrograms of the final sample were injected onto an analytical SEC column (YMC Diol 300) to determine its final purity. Additionally, sometimes SDS-PAGE electrophoresis was performed to determine final quality. Once all final metrics passed, the protein was sterilized in a biosafety cabinet using a sterile 0.22 micron filter. This was followed by sterile aliquoting and flash freezing in liquid nitrogen before storage at −80° C.

### Example 3

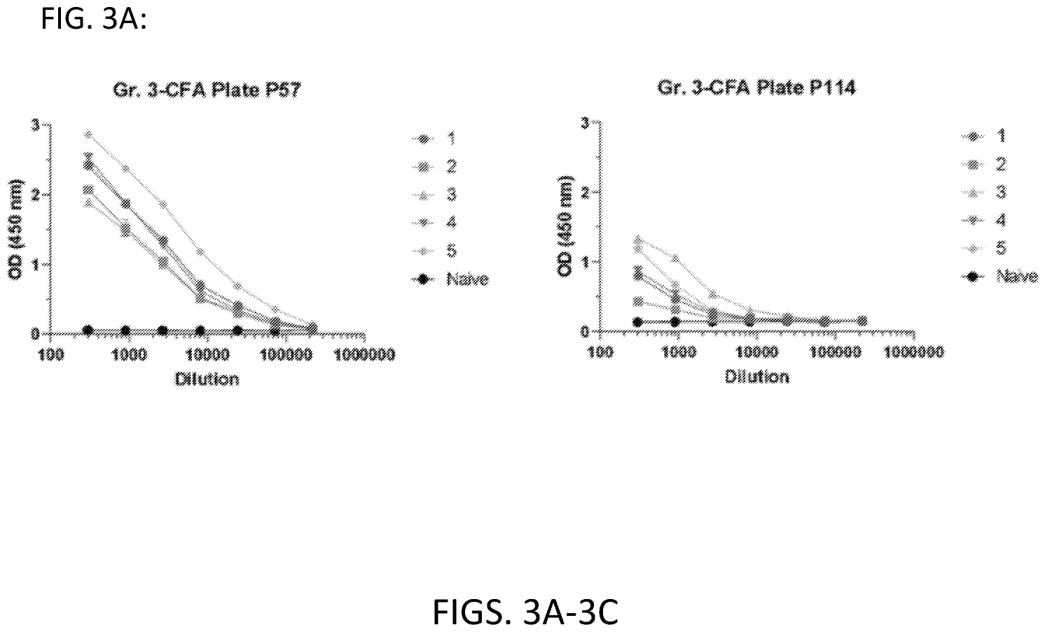

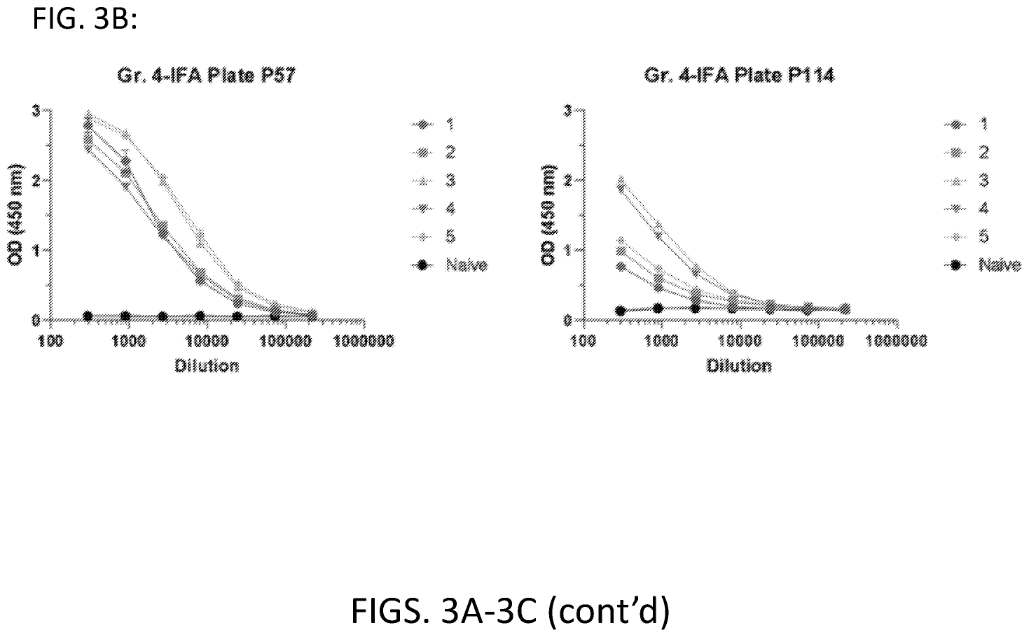

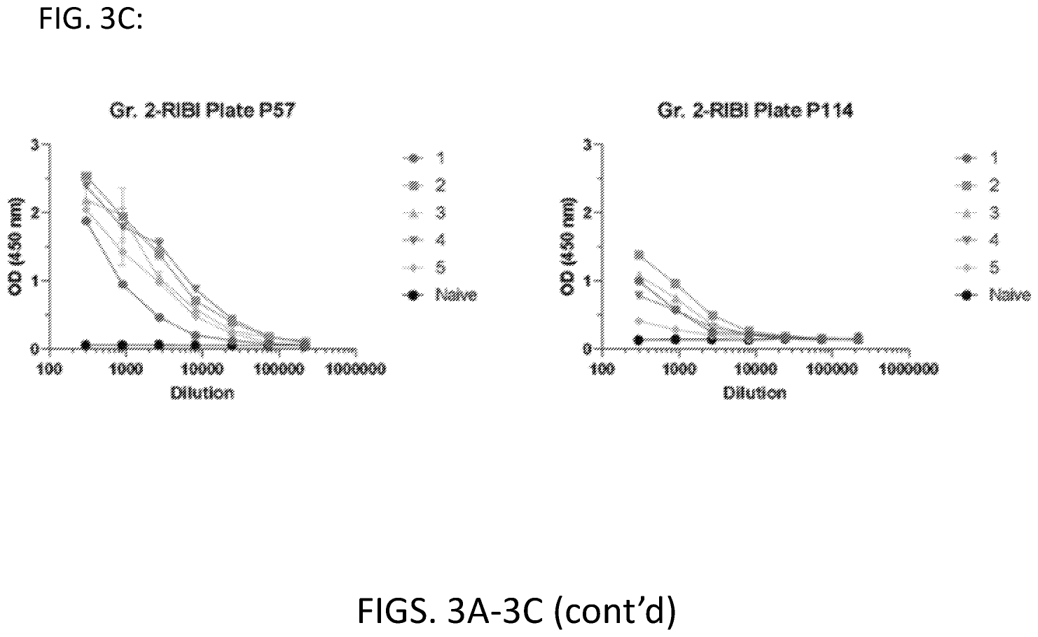

Recovery of CFHR4 antibody sequences from immunized mice. CFHR4 Immunization: Three cohorts of Alloy Therapeutic transgenic humanized mice (ATX-GK) were immunized with human CFHR4b using the following 5-week protocols (FIGS. 3A-3C):

Cohort 1:5 ATX-GK mice immunized with human CFHR4B (ATX-P-57) using standard 5-week RIMMS protocol of 10 ug subcutaneous dosing of antigen emulsified in complete Freund's adjuvant followed by 5 weekly subcutaneous dosing of antigen emulsified in incomplete Freund's adjuvant. Cohort 2:5 ATX-GK mice immunized with human CFHR4B (ATX-P-57) using 5-week RIMMS protocol of weekly 10 ug subcutaneous dosing of antigen emulsified in incomplete Freund's adjuvant. Cohort 3:5 ATX-GK mice immunized with human CFHR4B (ATX-P-57) using 5-week RIBI IP protocol of weekly 10 ug Intraperitoneal dosing of antigen emulsified in RIBI adjuvant.

Sample bleeds were taken at week four and tested for antigen positive serum titer and purification tag negative serum titer by ELISA. ELISA plates were coated with either 1 ug/ml of CFHR4 immunogen or an irrelevant protein (P114) with the same purification tag as the immunogen. Antigen coated plates were incubated with seven 10-fold serial dilutions of sera starting at 1:300. Antibodies bound to antigen were detected by anti-mouse IgG HRP secondary antibody and one step TMB solution. The absorbance signal at 450 nm was measured with an ELISA microplate reader.

Hybridoma: Immune tissues from high titer mice were harvested and preserved for antibody discovery. Hybridoma cell lines producing CFHR4 antibodies were produced by fusion of single B Cells from spleen and lymph nodes of titer positive mice with myeloma cells. Twenty 96 well plates of hybridoma fusions were generated and expanded. Hybridomas expressing CFHR4 specific antibodies were detected by antigen binding by ELISA. Affinity of antibodies in the hybridoma supernatants were measured by SPR using the Octet instrument. CFHR4 antibodies in hybridoma supernatant were loaded on a biosensor. Response was measured as a nm shift in the interference pattern and was proportional to the number of antibodies bound to the surface of the biosensor. The binding interaction of CFHR4 to the immobilized antibodies was measured as association (kon). Following analyte association, the biosensor was dipped into PBS without CFHR4, and the bound antigen was allowed to dissociate from the antibody (kdis). KD (M), or affinity of the antibodies for CFHR4 was measured as kdis/kon. Heavy and light chains from validated hybridomas were sequenced. RNA was isolated from CFHR4 antibody secreting hybridomas and heavy and light chain variable regions were cloned by reverse transcription using gene specific primers followed by PCR amplification with variable chain gene specific primers. PCR products were sequenced by standard Sanger sequencing methods.

Phage Display: Variable heavy and light chains were amplified from the spleen of high titer immunized mice by reverse transcription using gene specific primers followed by PCR amplification with variable chain gene specific primers. Variable regions were cloned into a phage display vector designed to express Fabs on phage g3p protein. Libraries of phage expressing unique Fabs were amplified and purified. Phage were allowed to bind to biotinylated CFHR4 antigens captured on streptavidin magnetic beads. Phage remaining bound to antigen beads after several stringent washes was eluted using a basic triethylamine solution and neutralized with Tris buffer pH 8.0. Eluted phages were reinfected into TG1 bacterial cells, amplified by co-infection with M13 helper phage, and purified by PEG precipitation. Purified phages expressing Fabs were selected for antigen binding as described. Phage from the second round were diluted and infected into TG1 cells. Polyclonal pools of phage output from two rounds of panning were tested by ELISA to confirm that the pools contained CFHR4-specific phage. Variable heavy and light chain regions were sequenced from single infected bacterial colonies using a rolling circle amplification and standard Sanger sequencing.

Antibody Sequencing: Unique variable heavy and light chain pairs from hybridoma and phage display campaigns were cloned into vectors designed to express full length antibodies as IgGs in HEK293 cells under the control of a CMV promoter. Antibody expression vectors were complexed with polyethylenimine and transfected into HEK293 cultures. After 5 days of shaking at 37° C. in 293 cell culture media, antibodies were captured on agarose-based protein A resin. After several stringent washes, antibodies were cluted in glycine solution, pH 3, neutralized with hepes, pH 9, and buffer exchanged into PBS.

### Example 4

Human CFHR4 monoclonal antibody differential scanning fluorimetry (DSF). Development of effective monoclonal antibodies depends not only on their biological activity but also on their physicochemical properties, such as homogeneity and stability. mAb stability can be affected by their formulation. Among the many techniques used to study the stability of mAbs, differential scanning fluorimetry (DSF) offers both excellent throughput and minimal material consumption. DSF measures the temperature of the protein unfolding transition (Tm) based on the change in fluorescence intensity of an environmentally sensitive dye.

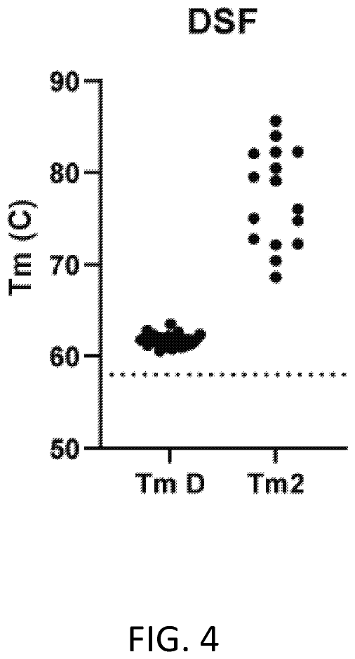

Experiments were conducted to assess the thermal stability of the human CFHR4 monoclonal antibodies (“ATX” antibodies) of the present disclosure by determining the melting temperature. Thermal stability was assessed via differential scanning fluorimetry (DSF) utilizing the Protein Thermal Shift (PTS) assay from Applied Biosystems. The assay was performed according to the manufacturer's instruction. Briefly, the antibody to be evaluated was prepared in triplicate by mixing with Protein Thermal Shift dye and buffer; a real-time melt experiment from 25° C. to 95° C. was run on QuantStudio 3. Data was analyzed by using Protein Thermal Shift Software and the melting temperature (Tm) was calculated from the melt curve (FIG. 4).

TABLE 2
DSF analysis of human anti-CFHR4 monoclonal antibodies.
Protein Thermal Shift Assay Results

| Sample ID | Tm D - Mean | Tm 2 - Mean |
|---|---|---|
| ATX-P-101 | 70.86 | |
| ATX-P-560 | 61.92 | |
| ATX-P-561 | 61.78 | 70.40 |
| ATX-P-562 | 62.34 | 72.79 |
| ATX-P-563 | 61.85 | |
| ATX-P-564 | 61.71 | |
| ATX-P-565 | 61.92 | 72.16 |
| ATX-P-566 | 61.36 | 75.03 |
| ATX-P-568 | 60.59 | |
| ATX-P-569 | 61.78 | |
| ATX-P-570 | 62.06 | 84.01 |
| ATX-P-572 | 61.78 | 82.26 |
| ATX-P-573 | 62.62 | |
| ATX-P-574 | 61.78 | |
| ATX-P-576 | 62.83 | |
| ATX-P-577 | 61.85 | |
| ATX-P-578 | 61.29 | |
| ATX-P-579 | 62.40 | |
| ATX-P-580 | 61.77 | |
| ATX-P-582 | 61.84 | 76.00 |
| ATX-P-583 | 61.56 | 72.22 |
| ATX-P-587 | 62.19 | 80.49 |
| ATX-P-588 | 61.42 | |
| ATX-P-591 | 61.56 | |
| ATX-P-592 | 61.21 | 82.10 |
| ATX-P-594 | 62.04 | |
| ATX-P-595 | 61.90 | |
| ATX-P-596 | 61.90 | 74.80 |
| ATX-P-597 | 63.51 | |
| ATX-P-598 | 60.99 | 68.63 |
| ATX-P-600 | 60.77 | 79.14 |
| ATX-P-602 | 61.34 | 85.66 |
| ATX-P-603 | 61.27 | |
| ATX-P-604 | 61.54 | |
| ATX-P-607 | 62.03 | |
| ATX-P-608 | 61.12 | |
| ATX-P-609 | 61.54 | 82.28 |

### Example 5

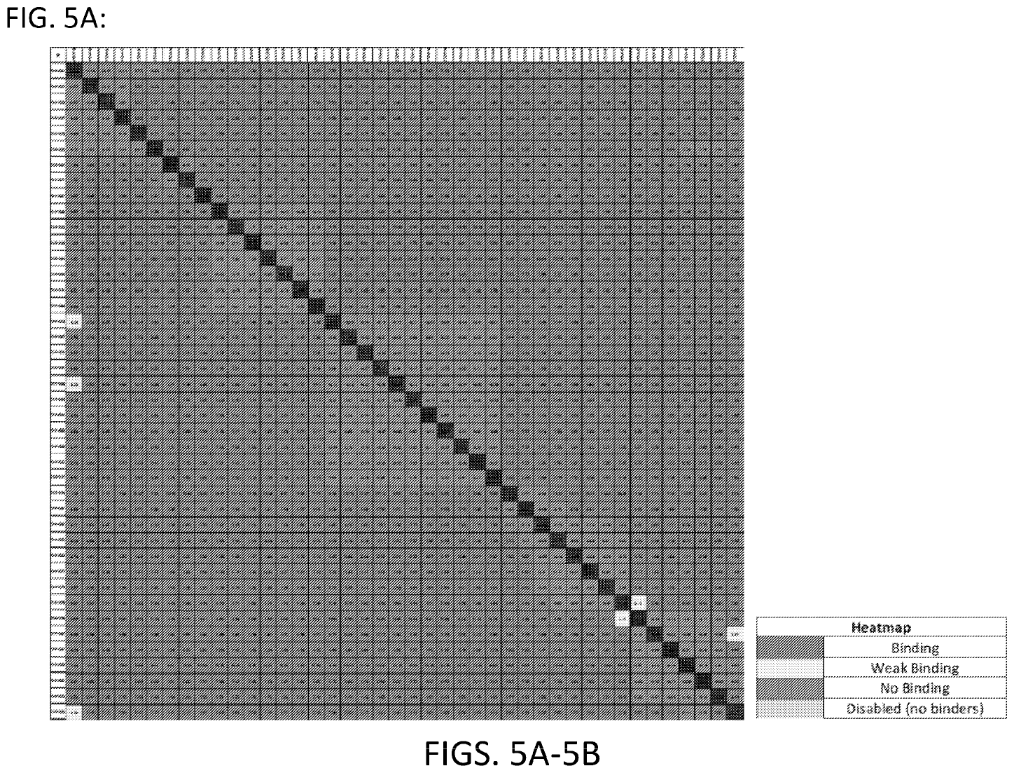

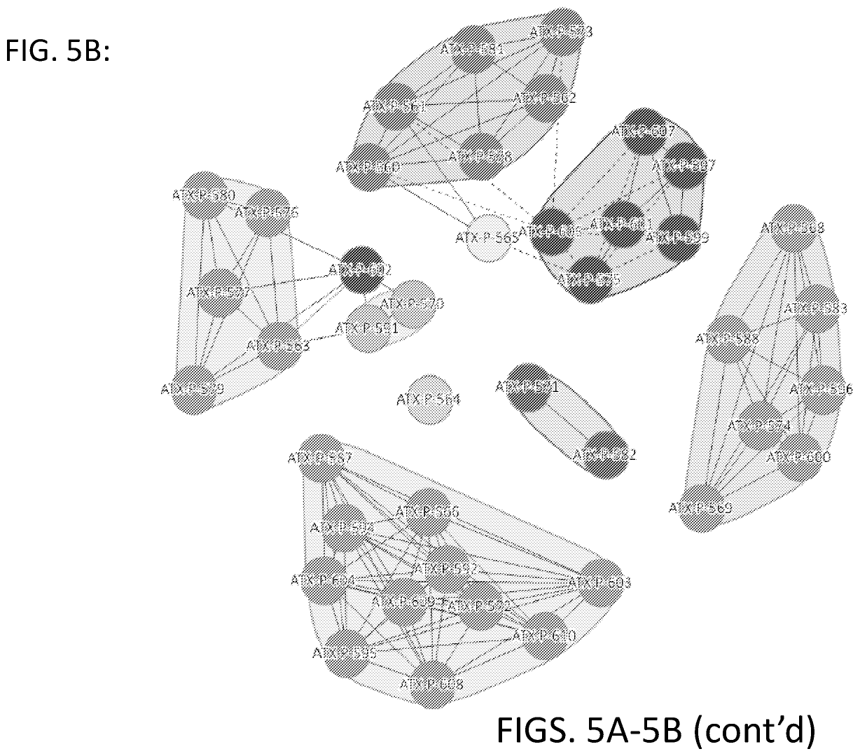

CFHR4 Antibody Cross-Blocking. High-throughput epitope binning experiments were conducted on real-time label-free biosensors (Carterra LSA) to sort large panels of mAbs into bins based on their ability to block one another for binding to the antigen. In a pairwise epitope binning analysis, antigen and antibody 2 (analyte antibody) are sequentially applied to the sensor chip (HC200M) covalently pre-loaded with antibody 1 (ligand antibody). An increase in response upon exposure to the analyte antibody indicates non-competition between the two antibodies, whereas a lack of change in the signal indicates competition. Antibodies having the same blocking profiles towards others in the test set are grouped into one bin. Community network plots are used to explore clustering of mAbs that share similar but not necessarily identical competition profiles. Rather than relying strictly on the sandwiching/blocking assignments in the heat map (FIG. 5A), as the Bin network plots do, hierarchical clustering is applied to the sorted heat map to generate network plots (FIG. 5B), which progressively group mAbs.

### Example 6

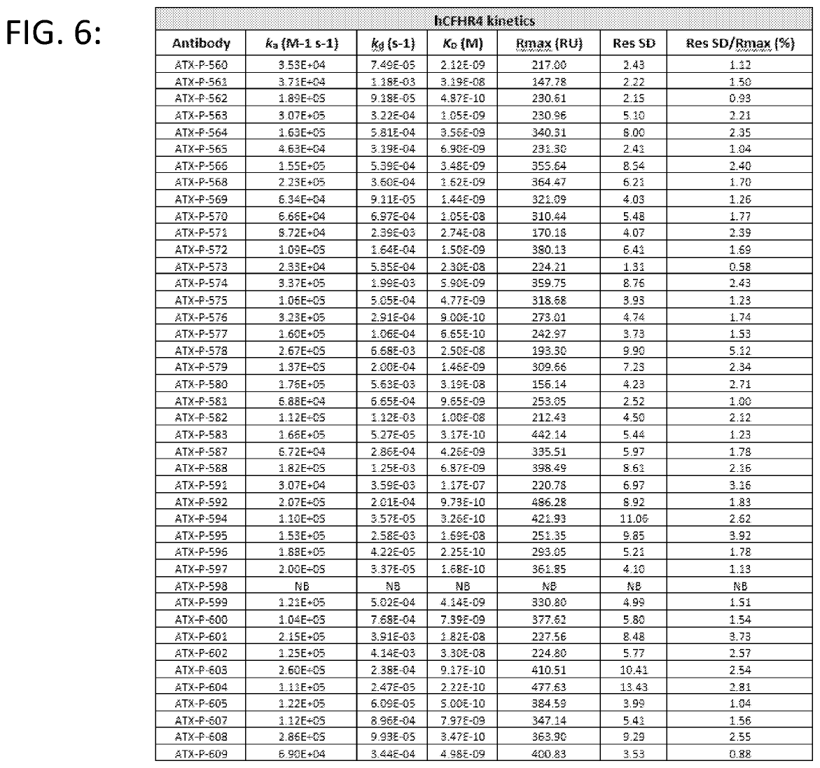

CFHR4 Antibody Binding Kinetics. Kinetic experiments were performed on Carterra LSA with a running buffer of PBS pH 7.40, 1% BSA, 0.05% Tween20. Antibodies were covalently printed on a HC30M chip. The chip was activated with 33 mM s-NHS and 133 mM EDC in 100 mM MES pH 5.5 for 7 minutes. Antibodies at 10 mg/ml in acetic acid buffer pH 4.5 were used for printing for 10 min. The printed chip was then quenched with 1 M ethanolamine pH 8.5 for 7 min. For kinetics analysis, purified recombinant his tagged protein ATX-P-57, human CDFR4B-His, at a concentration from 0.076 nM to 1500 nM (a serial 3-fold dilution) was injected sequentially. For each concentration, there was 5 min association followed by 15 min dissociation. Results were processed and analyzed in Carterra LSA Kinetics Software. The kinetic data was referenced with the interstitial reference spots and double-referenced to a buffer cycle, and then fit globally to a 1:1 binding model to determine their apparent association and dissociation kinetic rate constants (ka and kd values). The ratio kd/ka was used to derive the KD value of each antigen/mAb interaction, i.e., KD=kd/ka (FIG. 6).

### Example 7

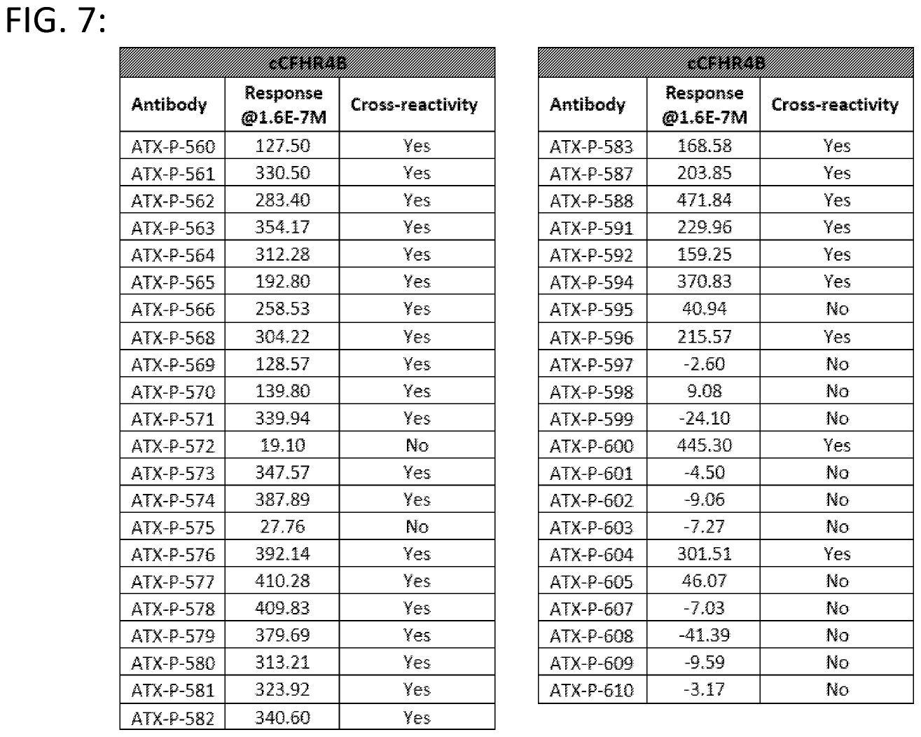

CFHR4 Antibody Cross Reactivity. Experiments were conducted to determine the reactivity of the CFHR4 antibodies of the present disclosure (developed using CFHR4b antigen; see Example 3) with various other CFHR4 proteins. As shown in FIG. 7, experiments were conducted to determine the reactivity of the CFHR4 antibodies with the Cynomolgus monkey CFHR4b protein (cCFHR4b). The binding experiments were performed on Carterra LSA with a running buffer of PBS pH 7.40, 1% BSA, 0.05% Tween20. Antibodies were covalently printed on a HC30M chip. Chip was activated with 33 mM s-NHS and 133 mM EDC in 100 mM MES pH 5.5 for 7 Min. Antibodies at 10 mg/ml in acetic acid buffer pH 4.5 were used for printing for 10 min. The printed chip was then quenched with 1 M ethanolamine pH 8.5 for 7 min. For the reactivity assay, ATX-P-141, cyno CFHR4B-mFc, at 160 nM was tested with 5 min association followed by 15 min dissociation. Results were processed and analyzed in Carterra LSA Kinetics Software. The data was referenced with the interstitial reference spots and double-referenced to a buffer cycle, and then the responses (nm) after association were reported. Isotype control was used to determine the cutoff response for positive binding.

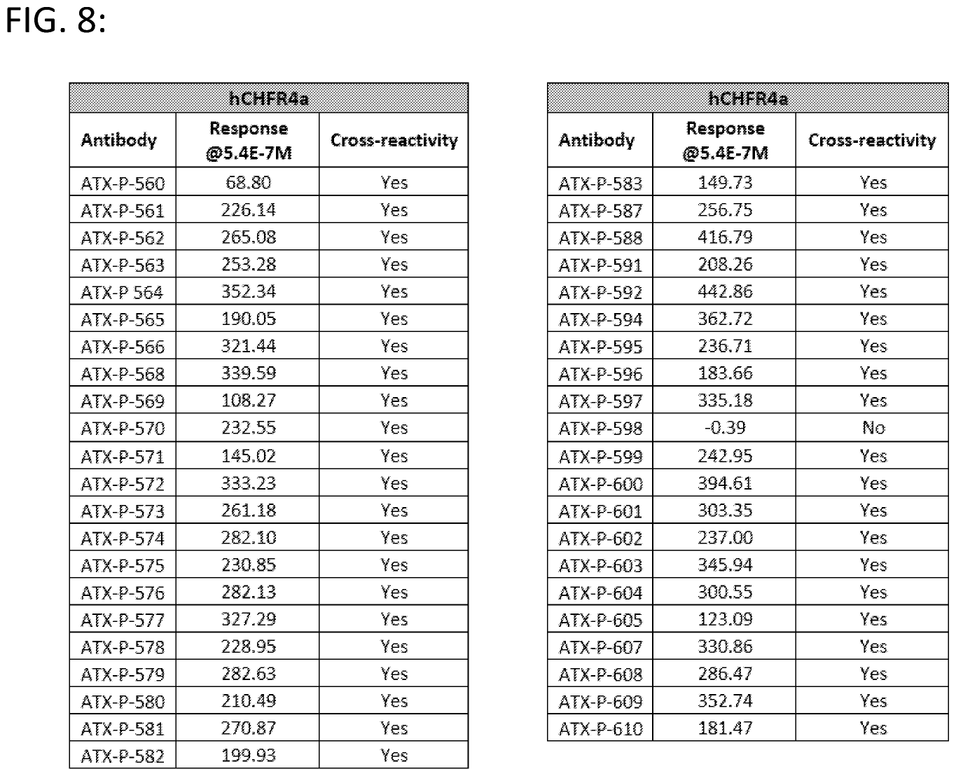

Experiments were also conducted to determine the reactivity of the CFHR4 antibodies with the human CFHR4a protein (FIG. 8). The binding experiments were performed on Carterra LSA with a running buffer of PBS pH 7.40, 1% BSA, 0.05% Tween20. Antibodies were covalently printed on a HC30M chip. Chip was activated with 33 mM s-NHS and 133 mM EDC in 100 mM MES pH 5.5 for 7 minutes. Antibodies at 10 mg/ml in acetic acid buffer pH 4.5 were used for printing for 10 min. The printed chip was then quenched with 1 M ethanolamine pH 8.5 for 7 min. For the reactivity assay, ATX-P-56, human CFHL4A-His, at 540 nM was tested with 5 min association followed by 15 min dissociation. Results were processed and analyzed in Carterra LSA Kinetics Software. The data was referenced with the interstitial reference spots and double-referenced to a buffer cycle, and then the responses (nm) after association were reported. Isotype control was used to determine the cutoff response for positive binding.

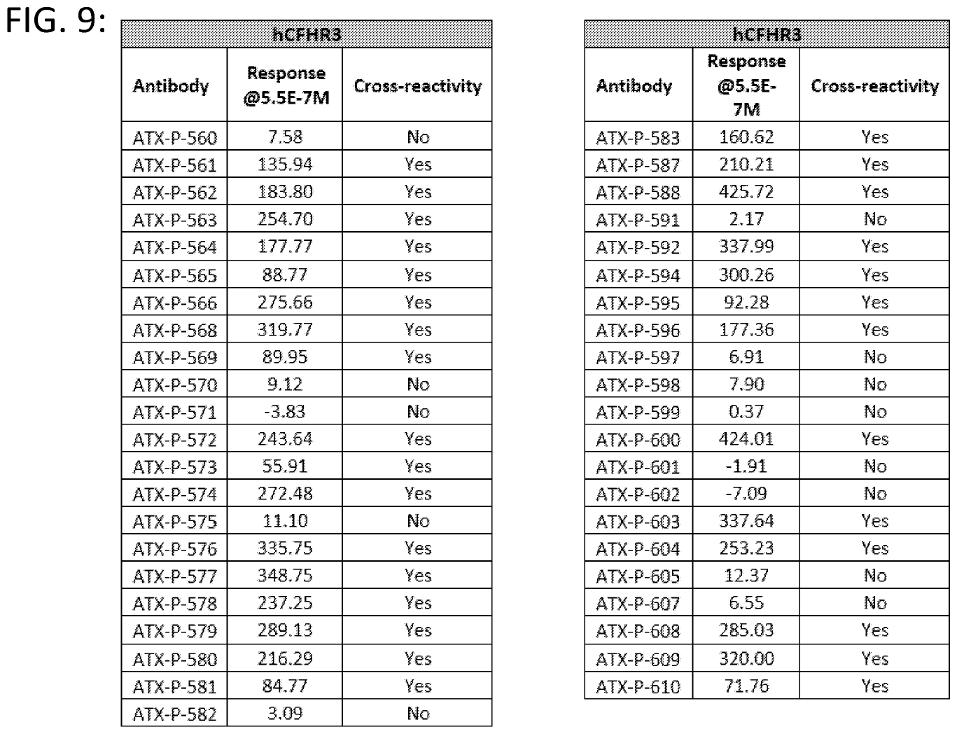

Experiments were also conducted to determine the reactivity of the CFHR4 antibodies with the human CFHR3 protein (FIG. 9). The binding experiments were performed on Carterra LSA with a running buffer of PBS pH 7.40, 1% BSA, 0.05% Tween20. Antibodies were covalently printed on a HC30M chip. Chip was activated with 33 mM s-NHS and 133 mM EDC in 100 mM MES pH 5.5 for 7 minutes. Antibodies at 10 mg/ml in acetic acid buffer pH 4.5 were used for printing for 10 min. The printed chip was then quenched with 1 M ethanolamine pH 8.5 for 7 min. For the reactivity assay, ATX-P-58, human CFHL3-His, at 550 nM was tested with 5 min association followed by 15 min dissociation. Results were processed and analyzed in Carterra LSA Kinetics Software. The data was referenced with the interstitial reference spots and double-referenced to a buffer cycle, and then the responses (nm) after association were reported. Isotype control was used to determine the cutoff response for positive binding.

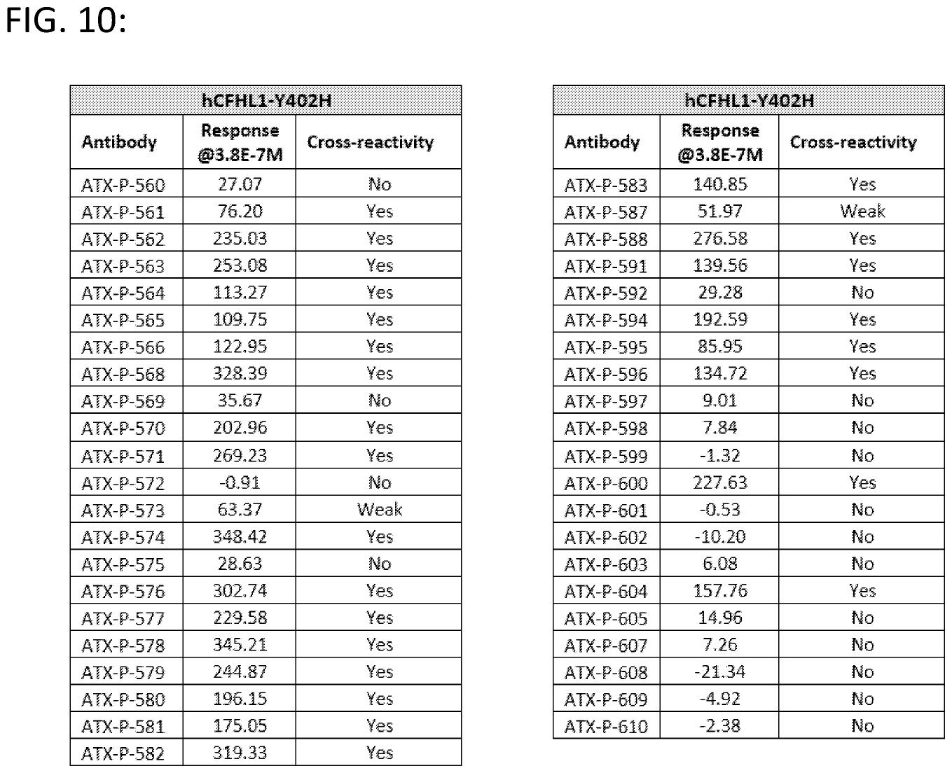

Experiments were also conducted to determine the reactivity of the CFHR4 antibodies with the human CFHL-1 (Y402H) protein (FIG. 10). The binding experiments were performed on Carterra LSA with a running buffer of PBS pH 7.40, 1% BSA, 0.05% Tween20. Antibodies were covalently printed on a HC30M chip. Chip was activated with 33 mM s-NHS and 133 mM EDC in 100 mM MES pH 5.5 for 7 minutes. Antibodies at 10 mg/ml in acetic acid buffer pH 4.5 were used for printing for 10 min. The printed chip was then quenched with 1 M ethanolamine pH 8.5 for 7 min. For the reactivity assay, ATX-P-421, human CFHL1-Y402H-His, at 380 nM was tested with 5 min association followed by 15 min dissociation. Results were processed and analyzed in Carterra LSA Kinetics Software. The data was referenced with the interstitial reference spots and double-referenced to a buffer cycle, and then the responses (nm) after association were reported. Isotype control was used to determine the cutoff response for positive binding.

### Example 8

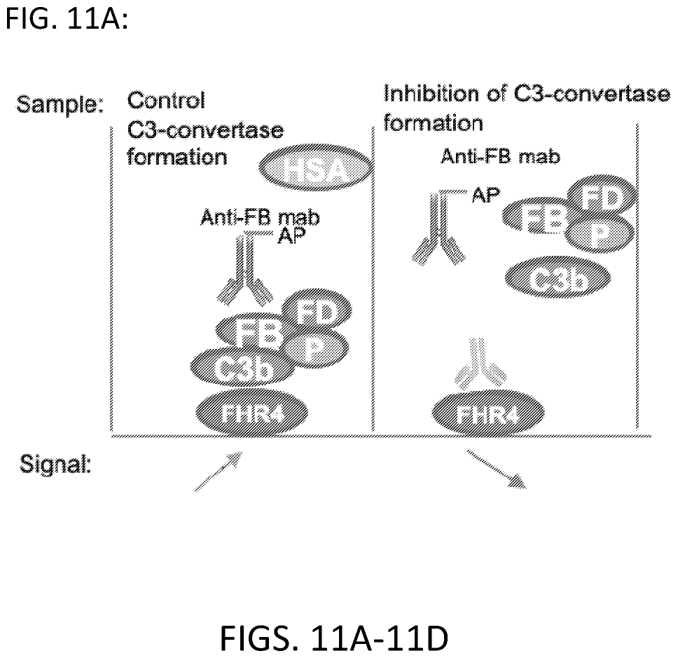

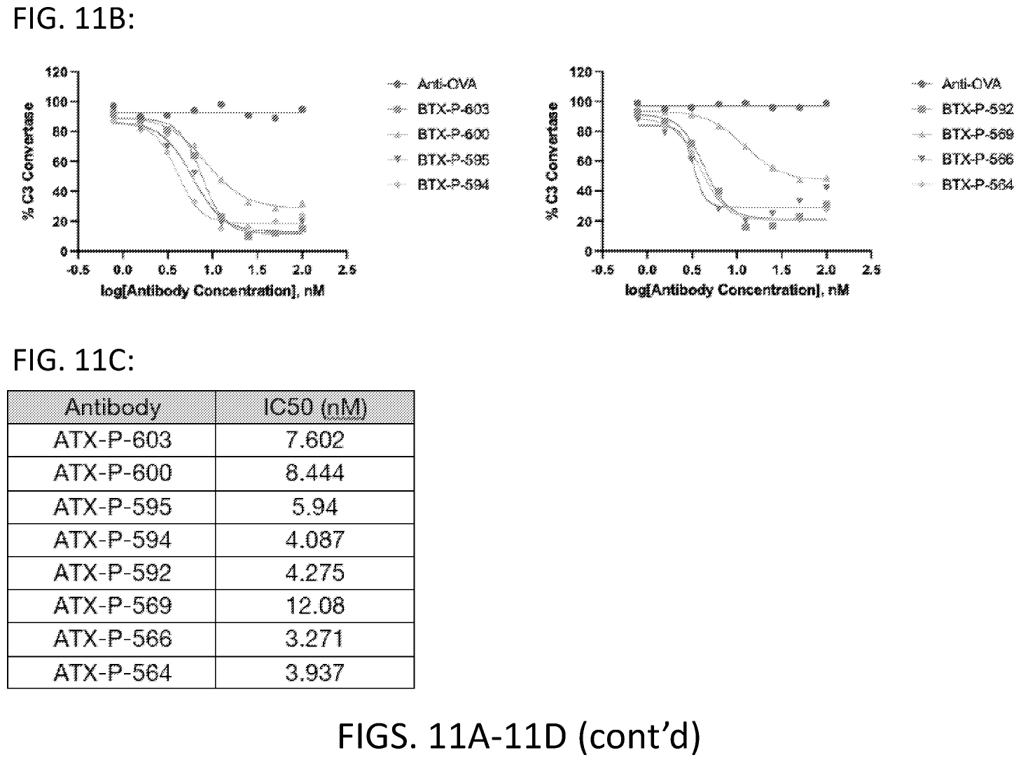

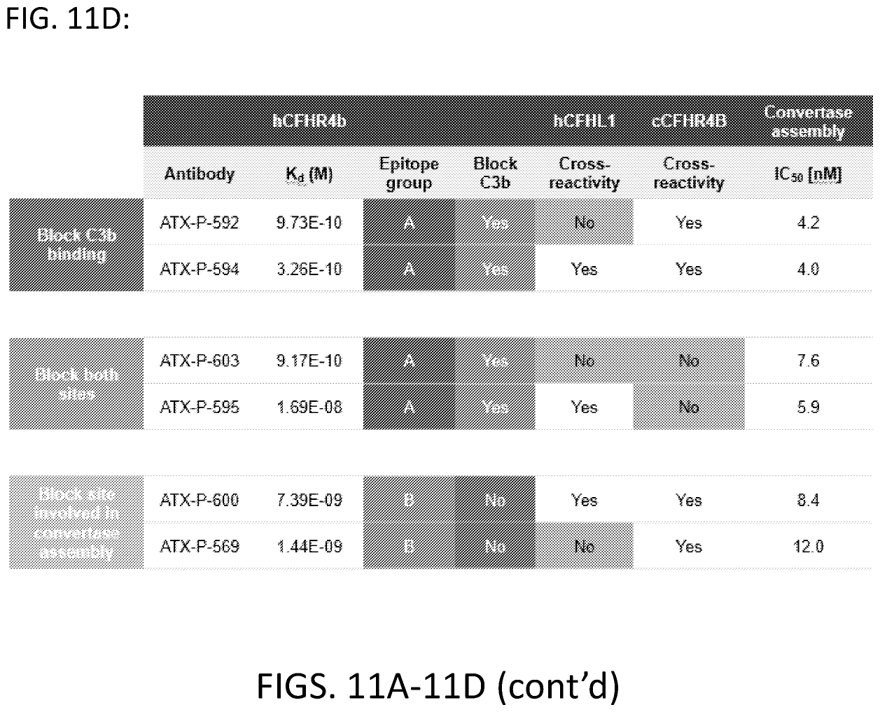

C3 Convertase Assembly assay. A screening assay was developed to evaluate the ability of the CFHR4 antibodies of the present disclosure to affect C3 convertase formation (FIG. 11A). C3bBb is the C3 convertase (EC 3.4.21.43) of the alternative pathway of complement activation. The C3 enzyme is responsible for amplification of pathway activation and for deposition on target cells of C3b and the membrane attack complex. It is controlled by the serum proteins Factor H, Factor I (EC 3.4.21.45), and properdin. The formation of C3 convertase requires C3b, Factor B, Factor D (EC 3.4.21.46), and Mg2+. After formation of the reversible, bimolecular complex C3bB (Mg), Factor D cleaves Factor B, releasing the activation fragment Ba, and generates the active enzyme C3bBb. The C3 enzyme is a serine protease whose catalytic site resides in the Bb subunit. Additionally, CFHR4 has been reported to directly promote C3 convertase formation by binding to C3b, with stronger ability to assemble C3bBb on CFHR4, and with C3bBb-CFHR4 complex being more resistant to CFH cleavage in comparison with C3bBb.

Thus, the assay is designed to test the ability of a CHFR4 antibody to disrupt the formation of the C3 convertase complex by measuring the amount of Factor B that is present using an anti-FB monoclonal antibody conjugated to a reporter moiety. As illustrated in FIG. 11A, in the presence of CFHR4 (absence of an effective CFHR4 antibody), the C3 convertase complex is formed and the anti-FB antibody binds FB and a signal is detected (i.e., complement activation). However, in the presence of an effective anti-CFHR4 antibody, the C3 convertase complex is disrupted and the antibody-bound FB is washed away, resulting in a decreased signal (i.e., no complement activation).

An example of the assay protocol is provided below:

Preparation of FHR-4 coated plates—(1) Coat plates with 5 μg/ml FHR-4 in PBS, incubate in plastic bag with moist paper O/N in RT. (2) Wash 3×300 μl with Wieslab wash buffer. (3) Block wells with 150 μl 3% BSA in PBS. (4) Incubate for a minimum of 1 h RT. (5) Aspirate wells and wash with Superblock 1×300 μl. (6) Dry plates at 37° C., 16% RH, for at least 1 h. (7) Store in plate bags in fridge.

Assay protocol—(1) Bring diluent, plate, substrate, and stop solution to RT. (2) Dilute anti-OVA antibody to desired concentrations in AP diluent. (3) Add 50 μl Anti-OVA, AP diluent (blank), and diluent control to the plate. (4) Incubate with lid for 1 h at RT. (5) Wash with Wieslab wash buffer 3×300 μl. (6) Add 50 μl C3b at 5 μg/ml or AP diluent to the plate. (7) Incubate the plate at 37° C. for 30 min. (8) Wash with Wieslab wash buffer 3×300 μl. (9) Add 50 μl protein mix (FB, FD, Properdin) to the plate. (10) Incubate the plate at 37° C. for 30 min. (11) Wash with Wieslab wash buffer 3×300 μl. (12) 50 μl anti-factor B antibody diluted 1:2000 in AP diluent were added to the whole plate. (13) Incubate the plate at RT for 1 h. (14) Wash with Wieslab wash buffer 3×300 μl. (15) Add 50 μl Rabbit anti-goat IgG-HRP antibody diluted 1:5000 in Stabilzyme to the whole plate. (16) Incubate the plate at RT for 1 h. (17) Wash with Wieslab wash buffer 3×300 μl. (18) 50 μl substrate were added to the whole plate. (19) Incubate the plate at RT for 20 min. (20) 50 μl stop solution were added to the whole plate. (21) The plate was read at 450-620 nm.

As shown in FIG. 11B, dose response curves were generated for the top 8 inhibitory antibodies screened in the C3 Convertase Assembly assay. The percent C3 Convertase activity is plotted relative to negative control (diluent only). FIG. 11C includes the IC50 values calculated for each antibody tested in FIG. 11B. And FIG. 11D provides a representative summary of data demonstrating at least three functional classes of anti-CFHR4 antibodies.

### Example 9

Recovery of C3 antibody sequences from immunized mice. C3 antibody sequences were recovered from immunized mice followed by phage display with either fixed or diverse light chains. Selected leads were produced as full aglyco IgG1s and extensively characterized.

C3 Recombinant Proteins. C3 proteins were procured from Complement Technologies (Tyler, TX); the proteins were purified directly from the relevant species (human or cynomolgus) sera. Recombinant proteins were qualified with analytic SEC and binding studies to established antibodies.

C3 Immunization. Two cohorts of Alloy Therapeutics transgenic humanized mice, ATX-GK Mix, were immunized with either human C3 or cynomolgus C3 using the following 5 week protocols: Cohort 1: 5 ATX-GX mix mice immunized with human C3 (REA23) using standard 5-week RIMMS protocol with 10 ug subcutaneous dosing of antigen. The first dose was emulsified in complete Freund's adjuvant with 4 follow on weekly 10 ug doses in incomplete Freund's adjuvant. Cohort 2: As Cohort 1 but with cynomolgus C3 (REA20).

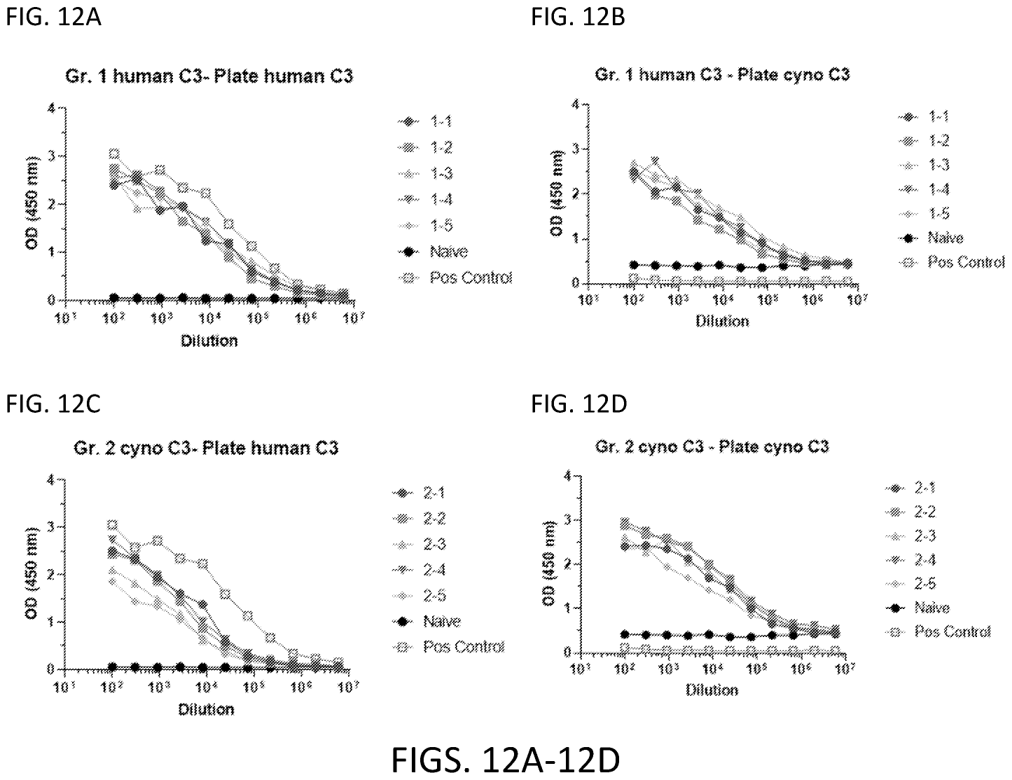

Sample bleeds were conducted at the fourth week and tested for antigen positive titer and cross reactive titer (human for cynomolgus cohort or cynomolgus for human cohort). ELISA plates were coated with 1 μg/ml of relevant recombinant protein overnight. Coated plates were incubated with sera across a descending 5×10-fold range starting at 1:100 and terminating at 1: 10E7 dilutions. Antibodies bound to the coated antigen were detected with anti-mouse IgG HRP secondary antibody and quantified with the aid of one step TMB solution and OD determination at 450 nM using a microplate reader. Note, the positive control antibody used for the ELISA experiment is not cross reactive to the cyno C3 protein (FIGS. 12A-12D).

Antibody Sequence Recovery—Phage Display. Spleen tissues from high titer mice were preserved at harvest for sequence recovery via phage display. RNA was extracted from said tissues. Two sequence recovery strategies were used in this study, a fixed light strategy and a diverse light chain strategy. For both strategies, variable heavy chains were amplified from the RNA prepared from the spleen tissues via reverse transcription. Following reverse transcription, viable gene specific primers and PCR were used to create DNA fragments which could be cloned into a phage display vector designed to express Fabs on phage g3p protein. For the diverse light chain strategy, the same approach was used to amplify the natural diversity of the light chain from the spleen RNA and clone into the Fab vector. For the fixed light chain strategy, the relevant part of the Fab vector was fixed to two specific sequences with constructs synthesized commercially that match the LC of ATX-P-569 and ATX-P-592.

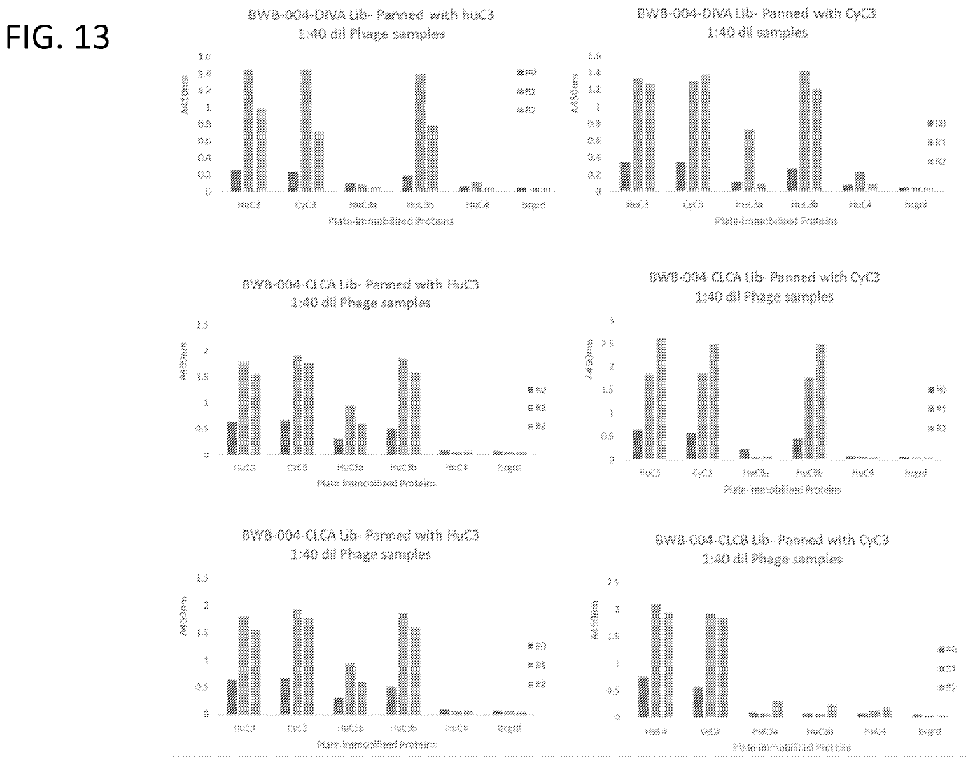

Once complete, the libraries of phage expressing unique Fabs or Fabs with fixed light chains were amplified, purified, and depleted on non-specific binders. Phage were allowed to bind to biotinylated C3 antigens (human and/or cyno) captured on streptavidin magnetic beads. Phage remaining bound to antigens beads after several stringent washes was eluted using a basic triethylamine solution and neutralized with Tris buffer pH 8.0. Eluted phages were reinfected into TG1 bacterial cells, amplified by co-infection with M12 helper phage, and purified by PEG precipitation. A small sample was saved for polyclonal analysis. Purified phages expressing Fabs were selected for antigen binding as described earlier in a subsequent 2nd round. Phage from the second round output were diluted and infected in TG1 cells. Polyclonal pools of phage output from both rounds were tested by ELISA to confirm enrichment and examine cross reactivity (FIG. 13).

Follow on monoclonal ELISA on individual colonies was conducted and from this second ELISA, clones were identified for rolling circle amplification and Sanger Sequencing of the variable heavy and light chain regions of the Fab where appropriate (for diverse libraries only, for fixed LC libraries only sequencing the HC was required). From the sequence analysis, a group of clones were advanced for reformatting into full length antibodies. The selected group broadly represented the clonotype diversity discovered in both the fixed/cLC and diverse libraries. In total, 113 paired variable heavy and light chain sequences were put into production across the fixed and diverse libraries.

Antibody Production. Unique variable heavy and light chain pairs from the phage display campaigns were cloned into vectors designed to express full length antibodies as algyco IgG1s in HEK293 cells under the control of a CMV promoter. Antibody expression vectors were complexed with polyethylenimine and transfected into HEK293 cultures. After 5 days of shaking at 37 C in 293 cell culture media, antibodies were captured on agarose-based protein A resin. After several stringent washes, antibodies were eluted in citrate solution, pH 3, neutralized with Hepes, pH 9, and buffer exchanged into PBS.

### Example 10

C3 Antibody Binding Kinetics. Experiments were conducted to determine the binding kinetics of anti-C3 antibodies, species cross-reactivity, and epitope cross-blocking.

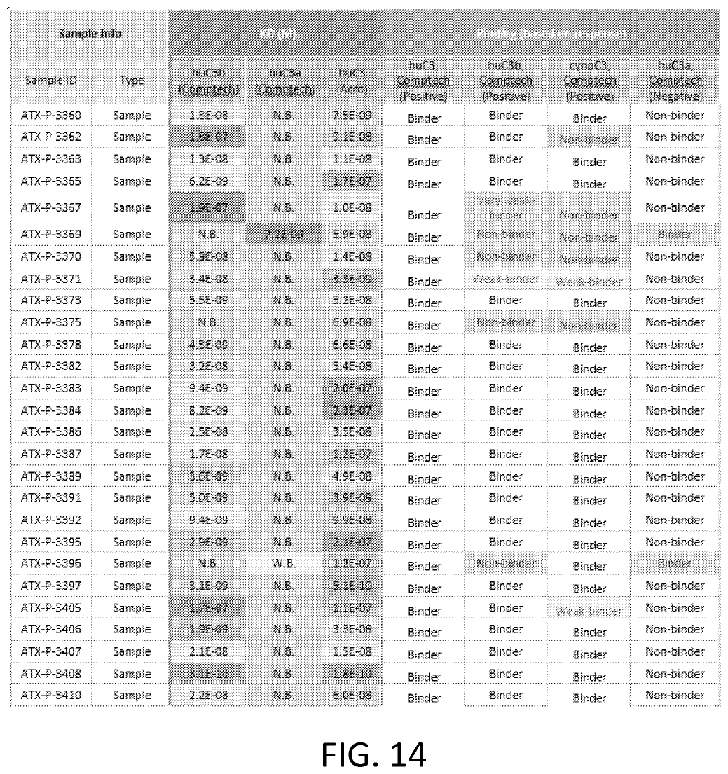

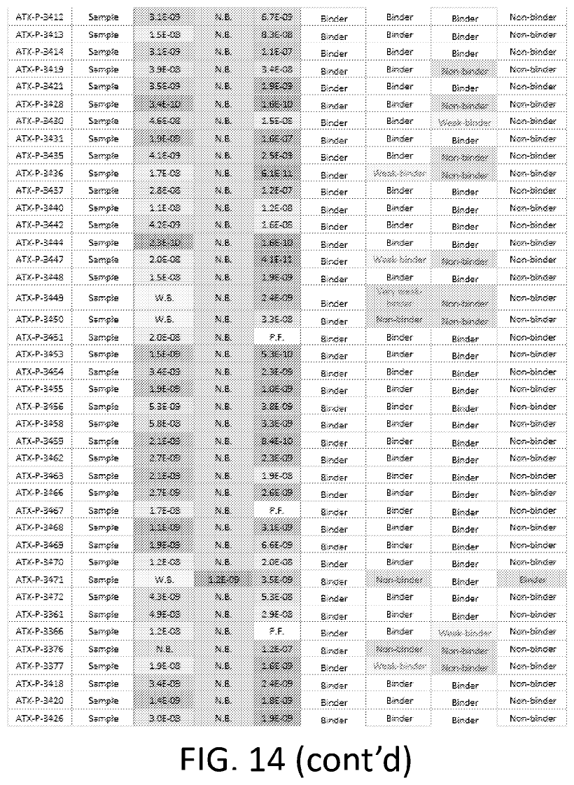

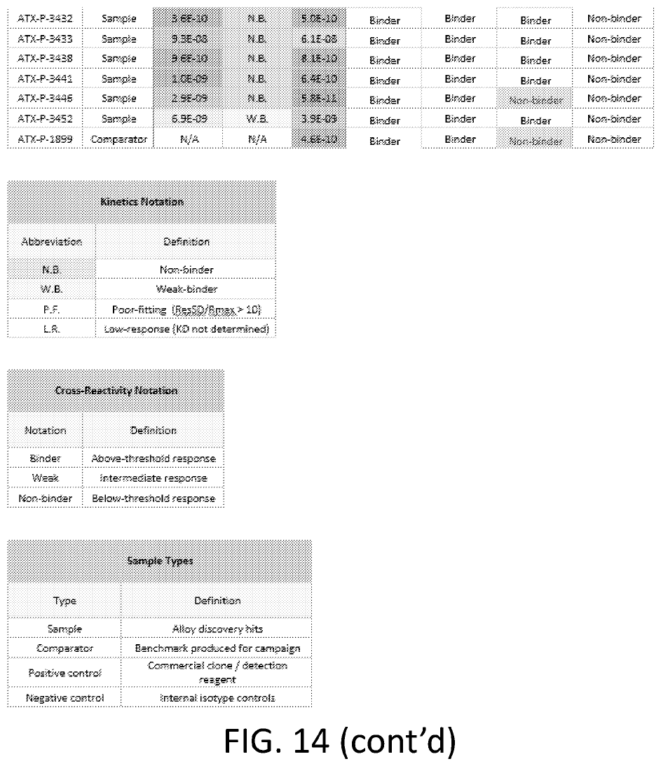

Kinetic experiments were performed on Carterra LSA with a running buffer of PBS pH 7.40, 1% BSA, 0.05% Tween20. Antibodies were captured on an anti-human Fc capture chip prepared with an HC30M chip. For kinetics analysis, C3a, C3b from Complement Technology, or C3-His from Acro Biosystems, were injected sequentially at a concentration from 0.076 nM to 1500 nM (a serial 3-fold dilution). For each concentration, there was a 5 Min association followed by 15 Min dissociation. Results were processed and analyzed in Carterra LSA Kinetics Software. The kinetic data was referenced with the interstitial reference spots and double-referenced to a buffer cycle, and then fit globally to a 1:1 binding model to determine their apparent association and dissociation kinetic rate constants (Kₐ and K_{D} values). The ratio K_{D}/Kₐ was used to derive the K_{D} value of each antigen/mAb interaction (K_{D}=K_{D}/Kₐ) (FIG. 14).

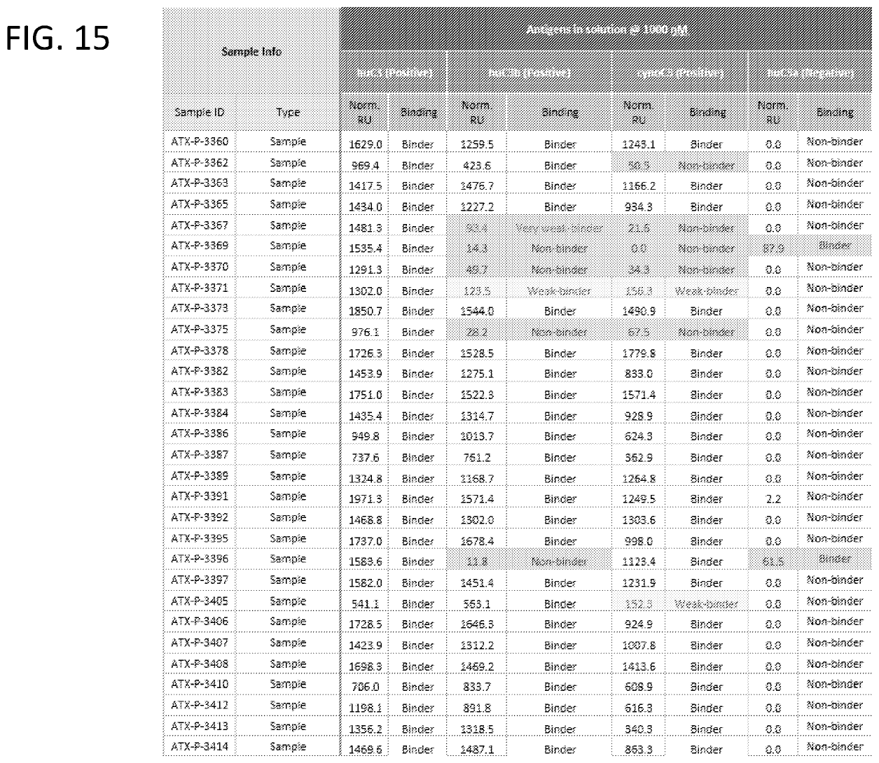

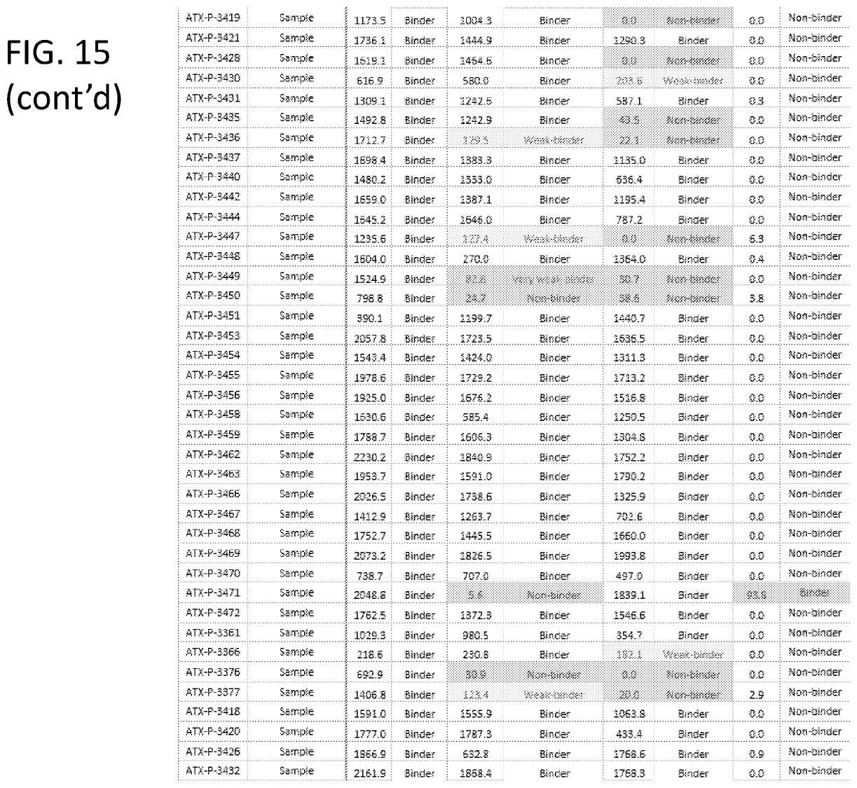

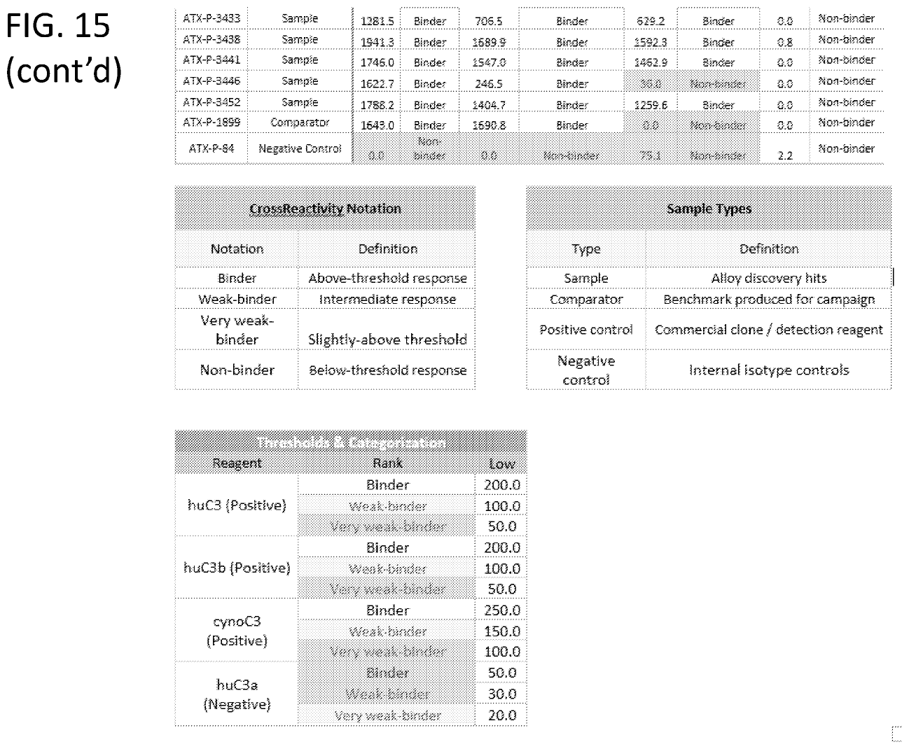

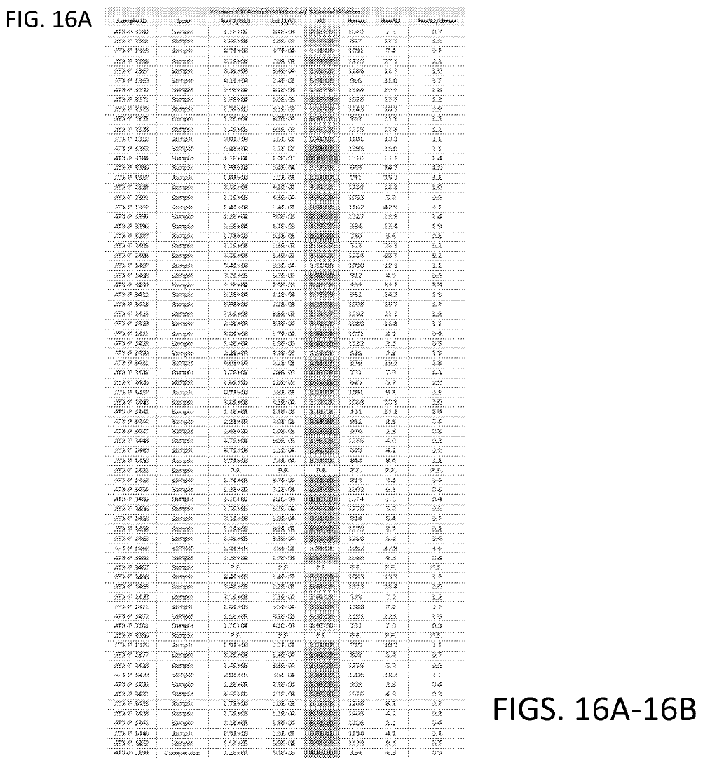

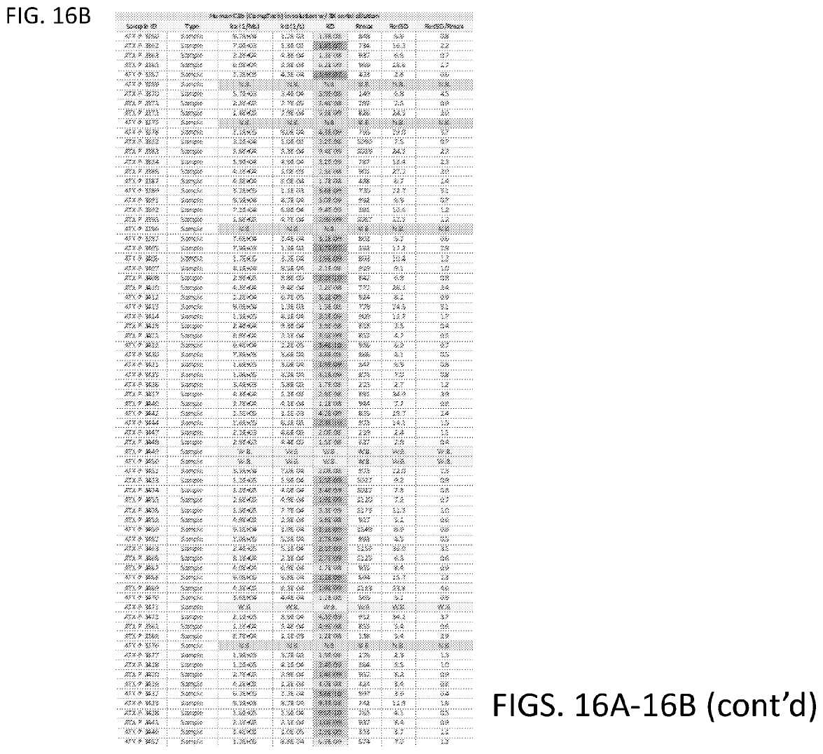

Cross-reactivity assay by SPRi. The binding experiments were performed on Carterra LSA with a running buffer of PBS pH 7.40, 1% BSA, 0.05% Tween20. Antibodies were covalently printed on an HC30M chip. Chip was activated with 33 mM s-NHS and 133 mM EDC in 100 mM MES pH 5.5 for 7 minutes. Antibodies at 10 mg/ml in acetic acid buffer pH 4.5 were used for printing for 10 min. The printed chip was then quenched with 1 M ethanolamine pH 8.5 for 7 min. For the reactivity assay, human C3, C3a, C3b, and cyno C3 from Complement Technology was tested with 5 Min association followed by 15 Min dissociation. Results were processed and analyzed in Carterra LSA Kinetics Software. The data was referenced with the interstitial reference spots and double-referenced to a buffer cycle, and then the responses (nm) after association were reported. Isotype control was used to determine the cutoff response for positive binding (FIGS. 15 and 16).

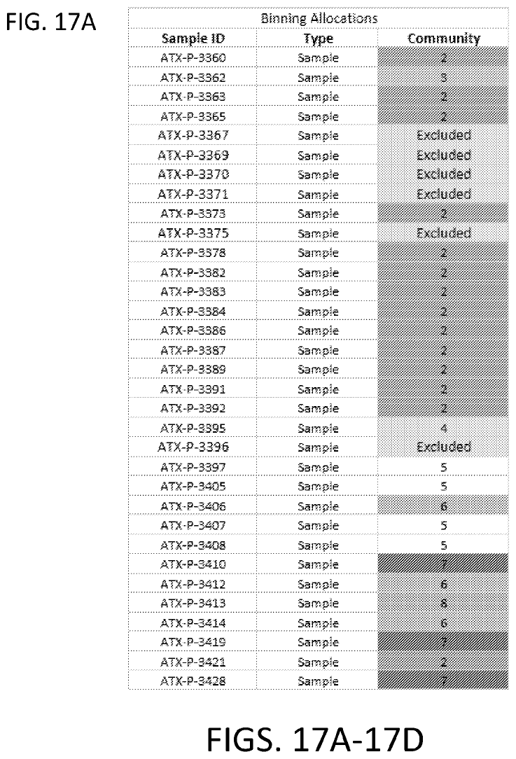

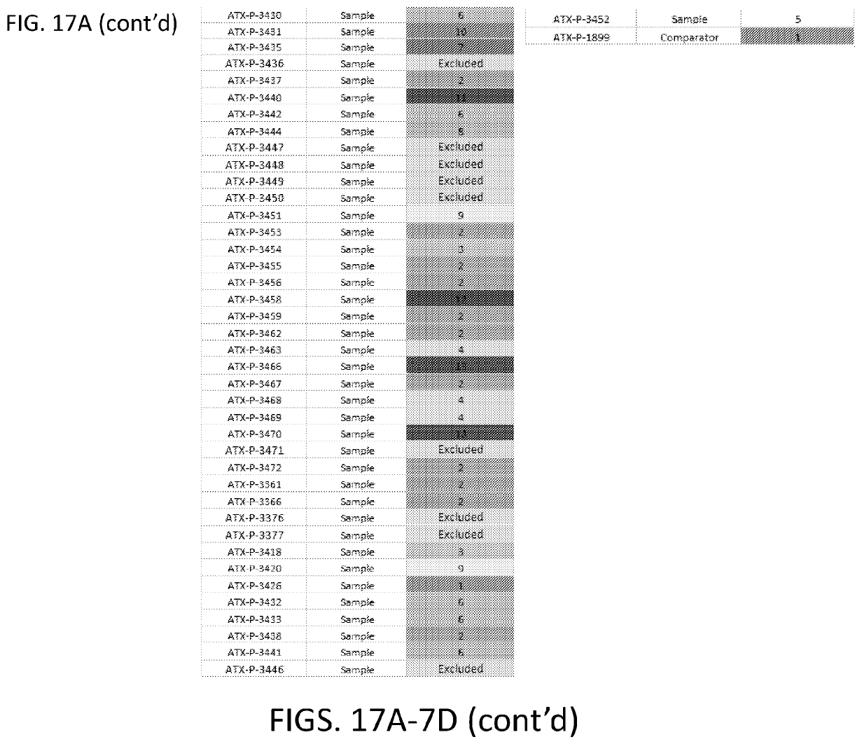

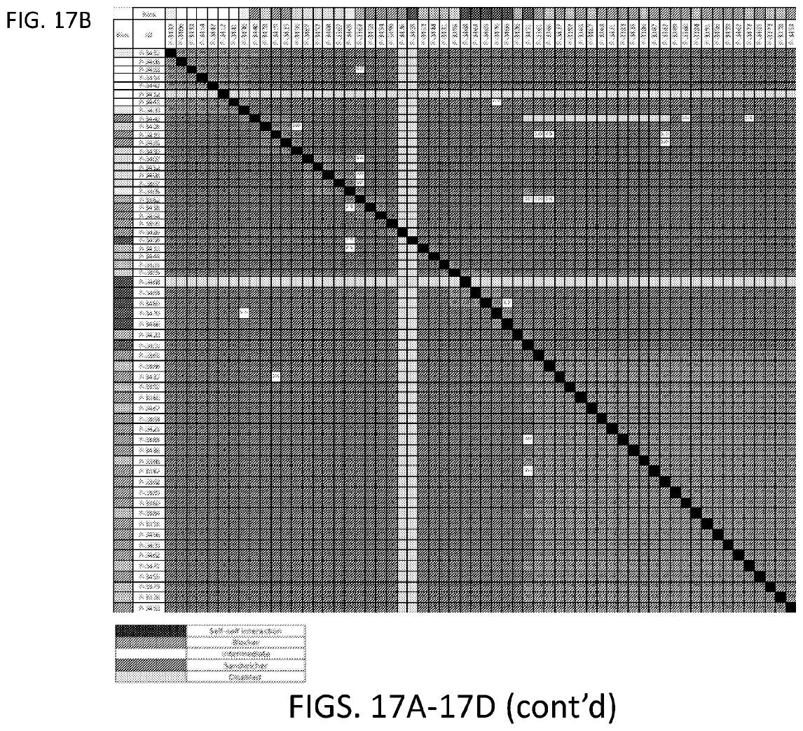

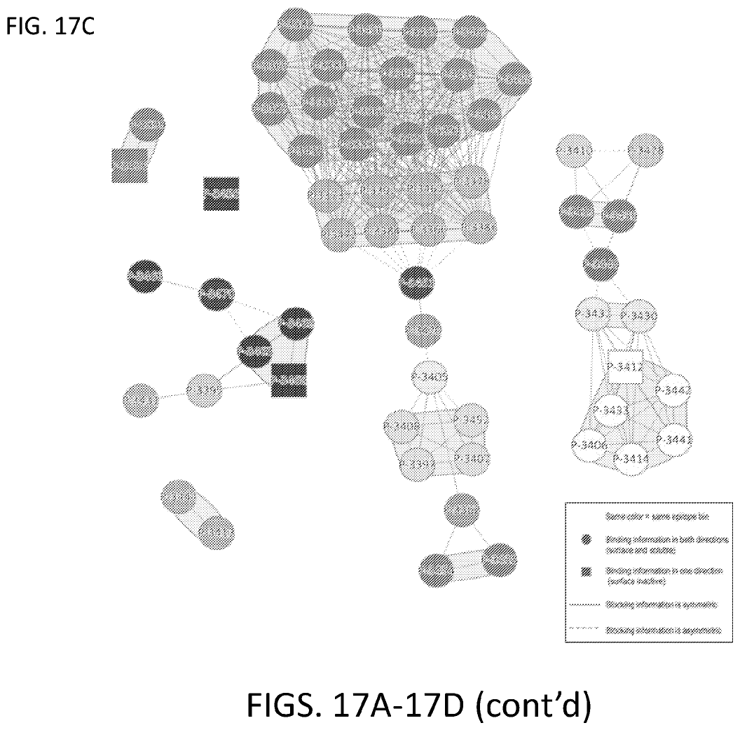

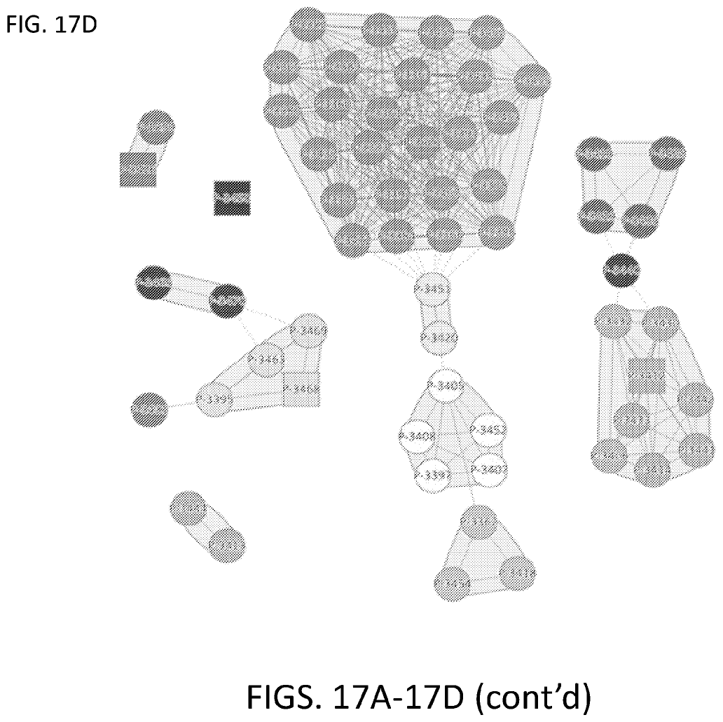

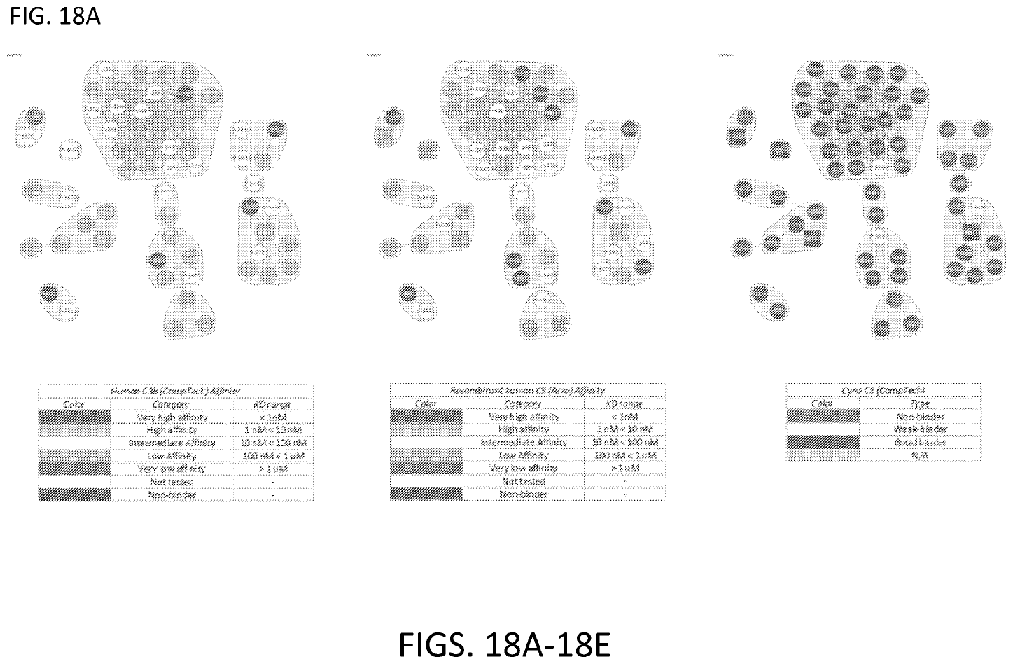

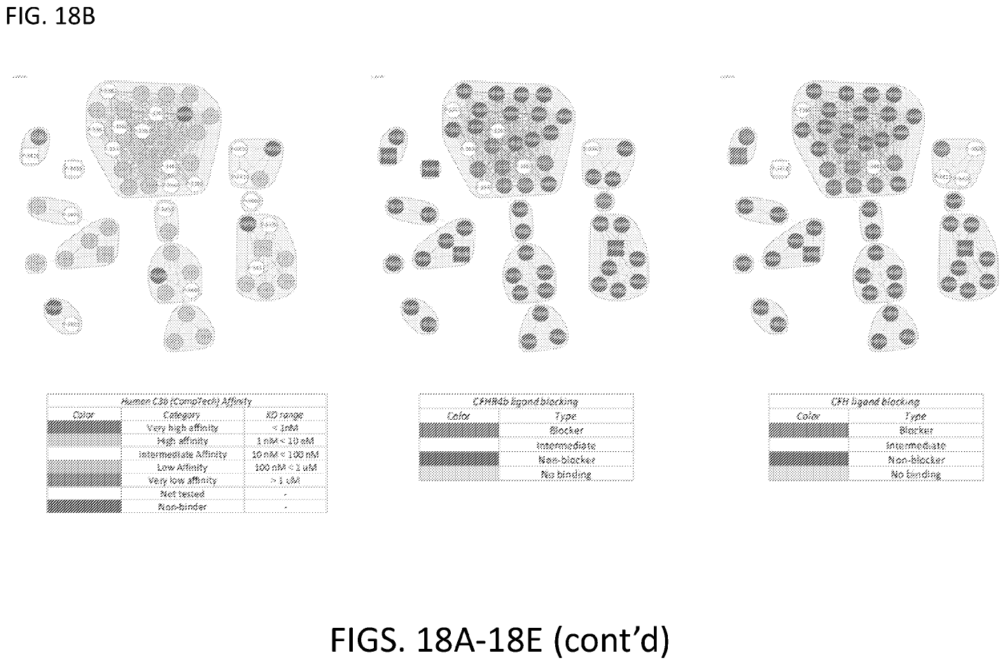

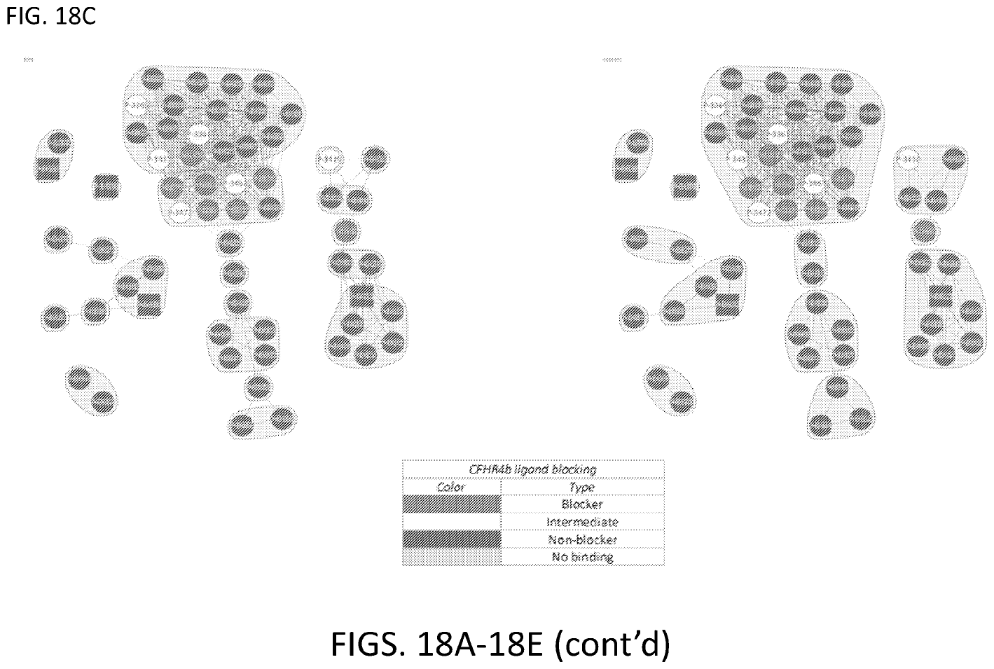

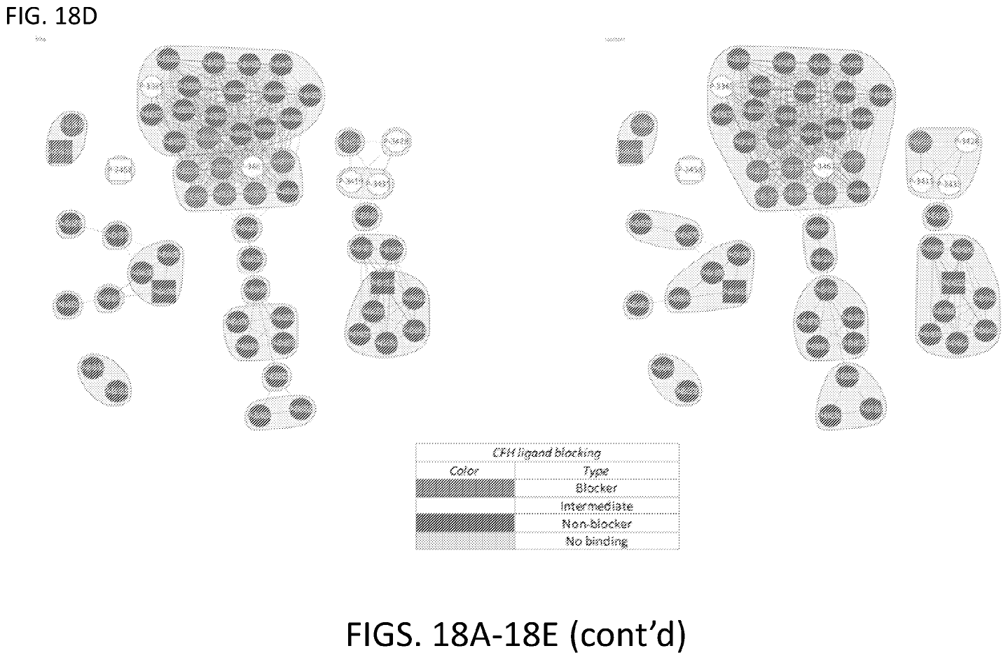

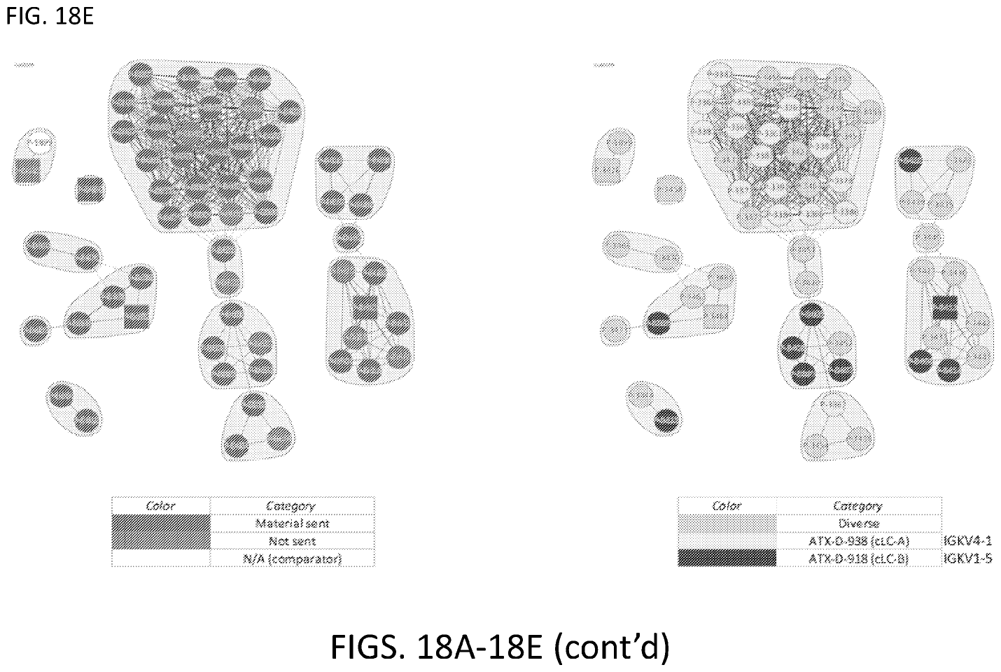

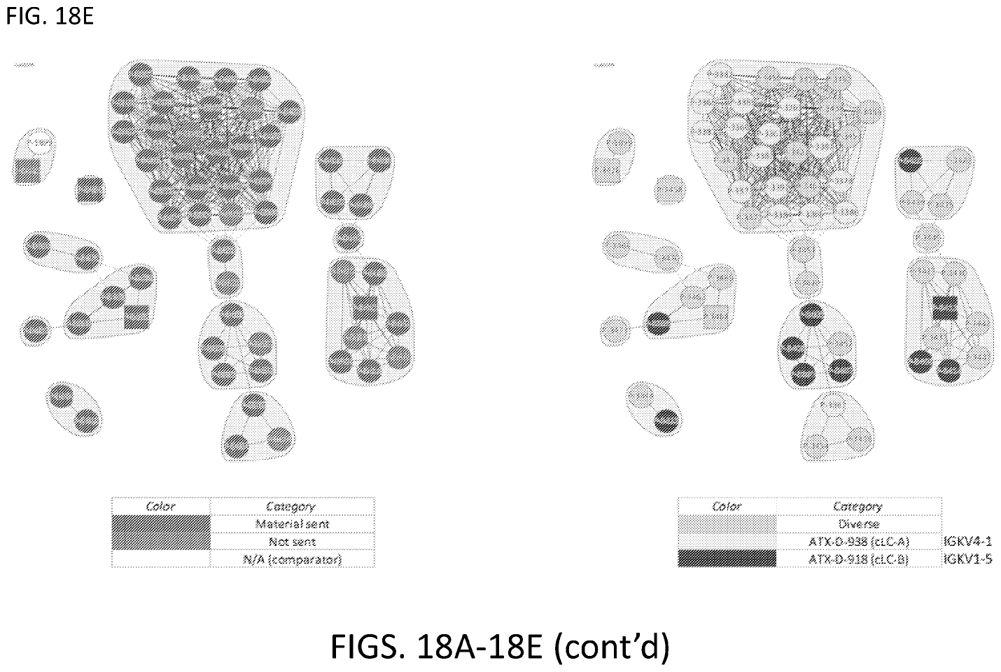

Epitope Binning. High-throughput epitope binning experiment was done on real-time label-free biosensors (Carterra LSA) to sort large panels of mAbs into bins based on their ability to block one another for binding to the antigen. In a pairwise epitope binning analysis, antigen and antibody 2 (analyte antibody) are sequentially applied to the sensor chip (HC200M) covalently pre-loaded with antibody 1 (ligand antibody). An increase in response upon exposure to the analyte antibody indicates non-competition between the two antibodies, whereas a lack of change in the signal indicates competition. Antibodies having the same blocking profiles towards others in the test set are grouped into one bin. Community network plots are used to explore clustering of mAbs that share similar but not necessarily identical competition profiles. Rather than relying strictly on the sandwiching/blocking assignments in the heat map, as the Bin network plots do, hierarchical clustering is applied to the sorted heat map to generate dendrograms, which progressively group mAbs (FIGS. 17-18).

### Example 11

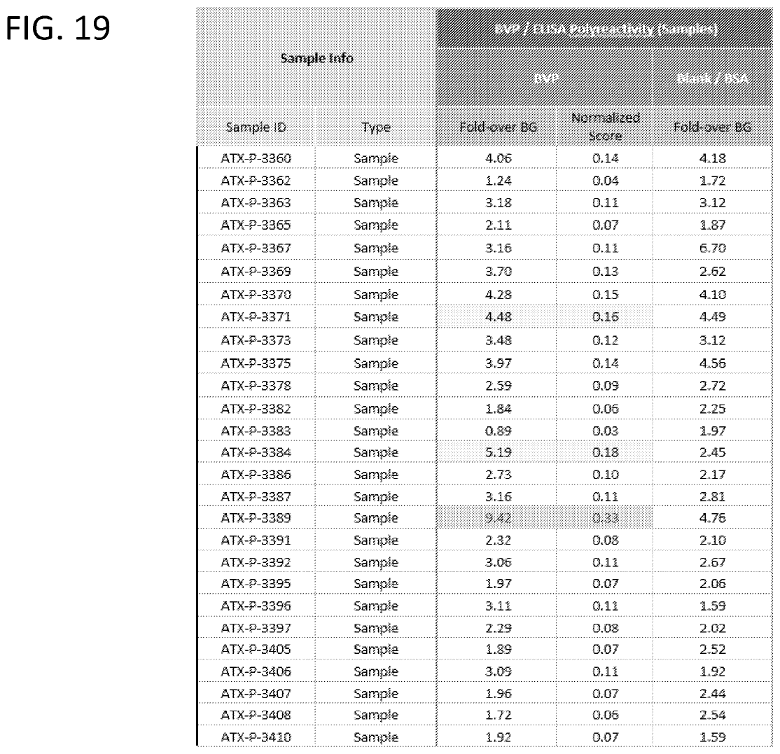

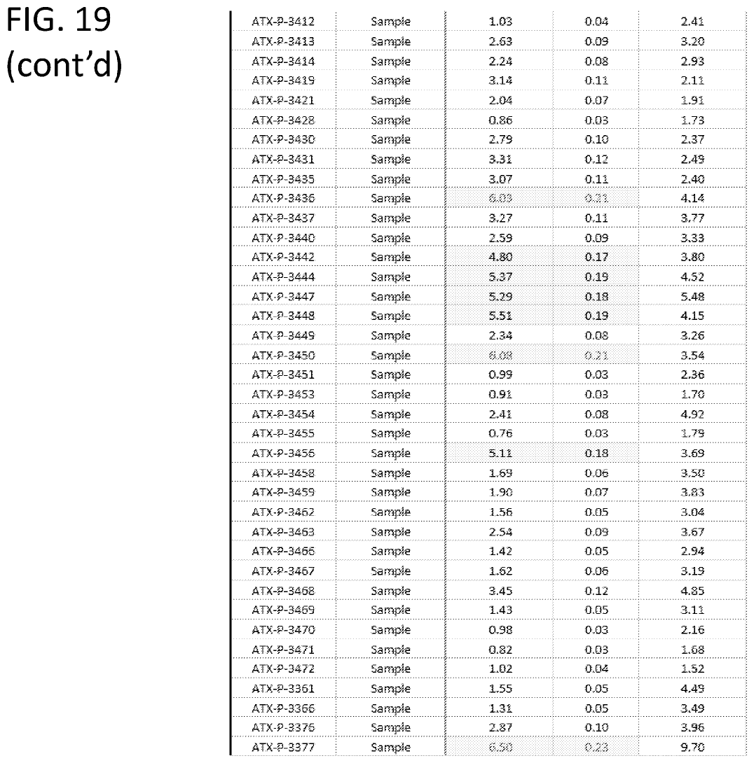

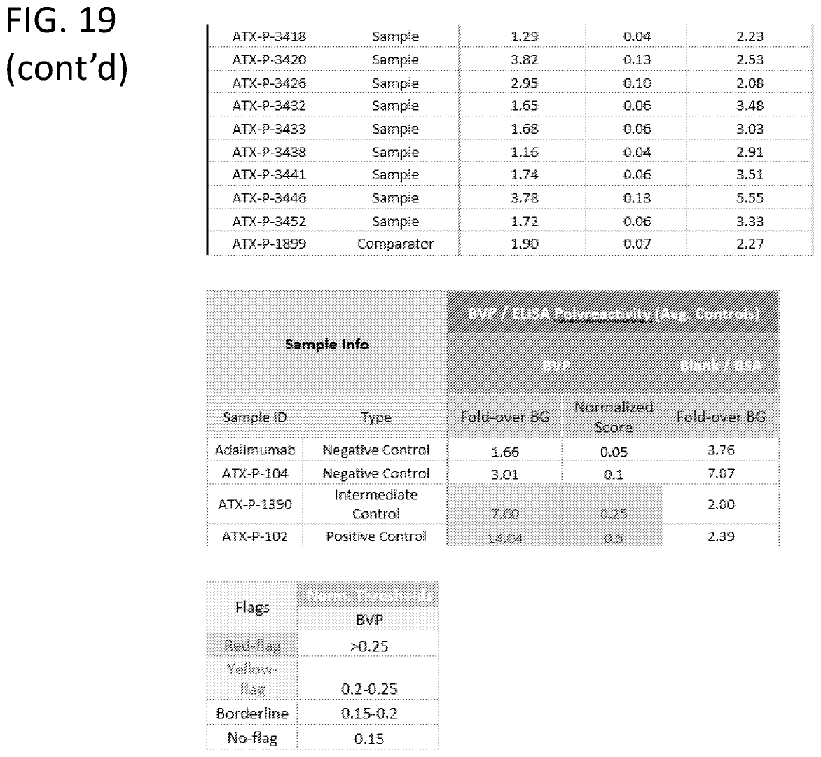

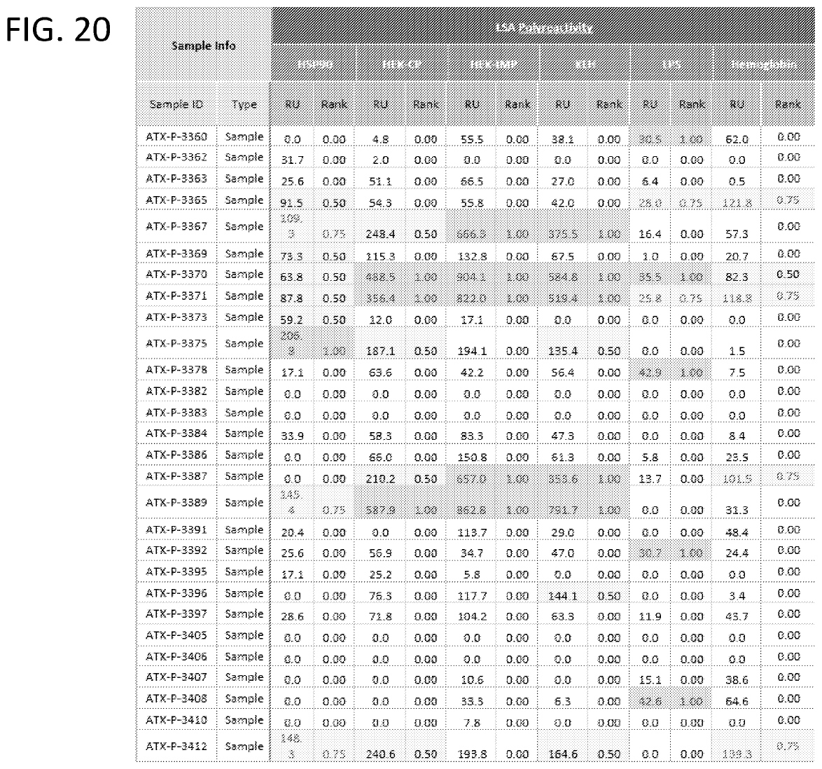

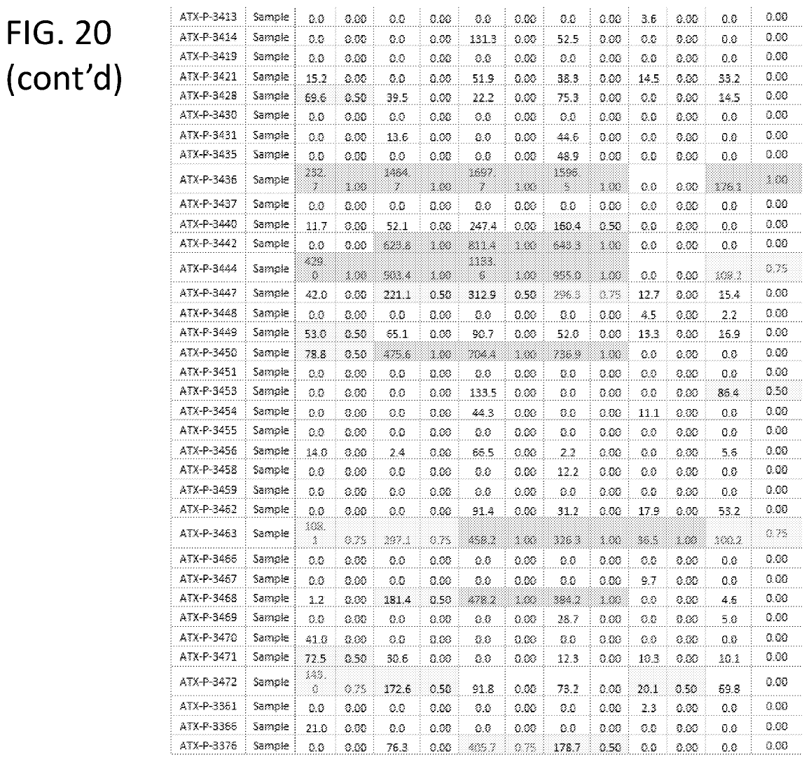

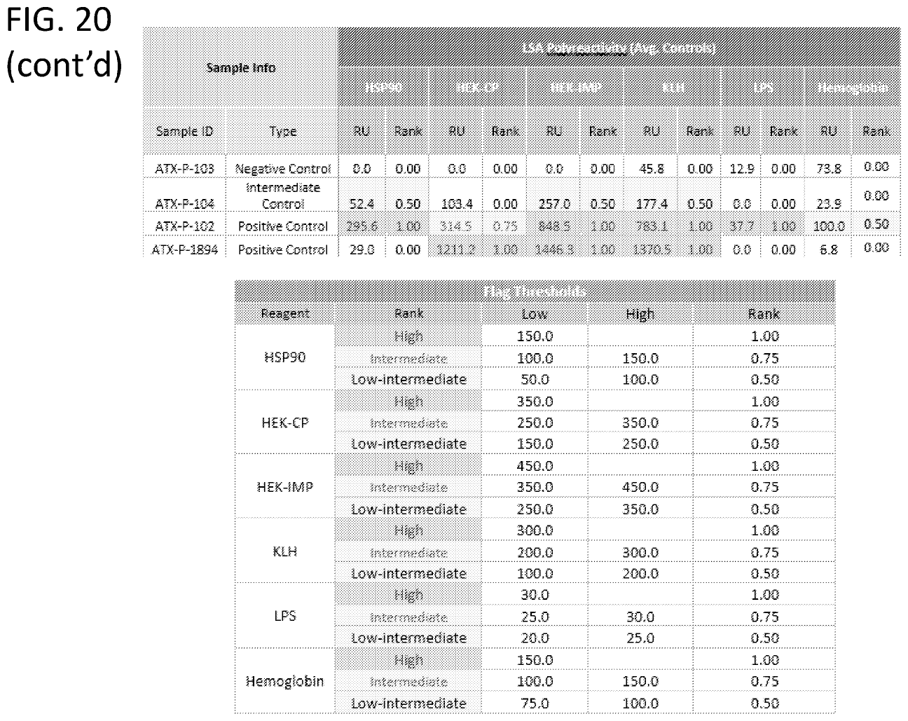

C3 Antibody Polyreactivity. Experiments were conducted to determine the reactivity of the C3 antibodies of the present disclosure with various other proteins using ELISA and Carterra LSA platforms (FIGS. 19 and 20). The methods used were similar to those reported by Hötzel et al. (2012). Briefly, baculovirus particles (BVP, Lake Pharma) was diluted 1:100 in 50 mM sodium bicarbonate (pH 9.3). After overnight incubation of 50 μL of BVP on ELISA plates (3369; Corning) at 4° C. overnight, unbound BVPs were aspirated from the wells. All remaining steps were performed at RT. The plate was blocked with 100 μL of blocking buffer (PBS with 1% BSA) for 1 h before three washes with 100 μL of PBS. Next, 50 μL of 16 nM testing antibodies was added to the wells and incubated for 1 h followed by washes with 100 μL of PBS. HRP-conjugated goat anti-human IgG antibody at 1:1000 (Jackson ImmunoResearch) was used as the secondary antibody, and incubated for 1 h followed by washes as before. Finally, 50 μL of TMB substrate (34021; Fisher Scientific) was added to each well and incubated for 10-15 min. The reactions were stopped by adding 50 μL of 2 M sulfuric acid to each well. The absorbance was read at 450 nm and BVP score determined by normalizing absorbance by control wells with no test antibody.

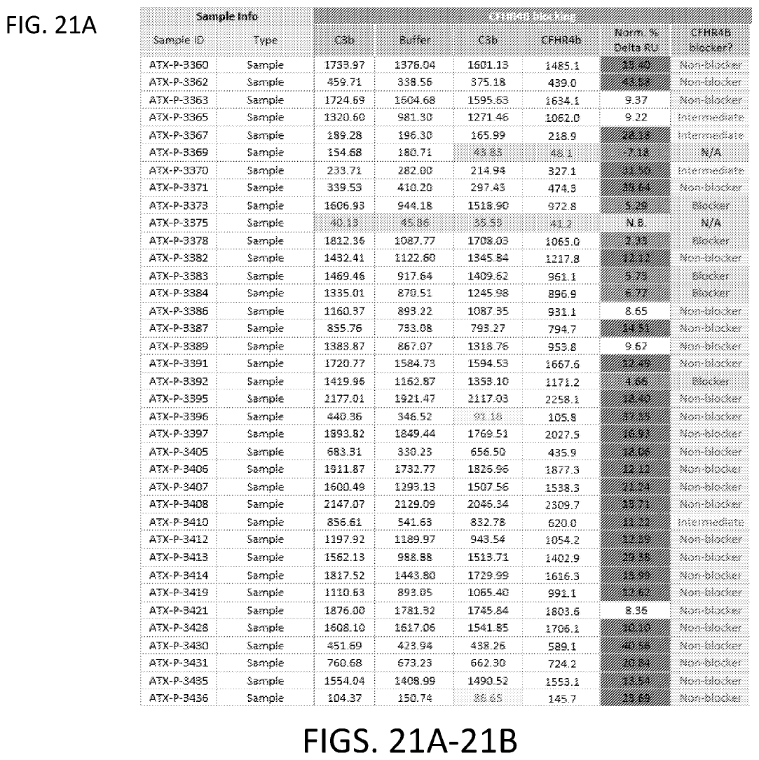

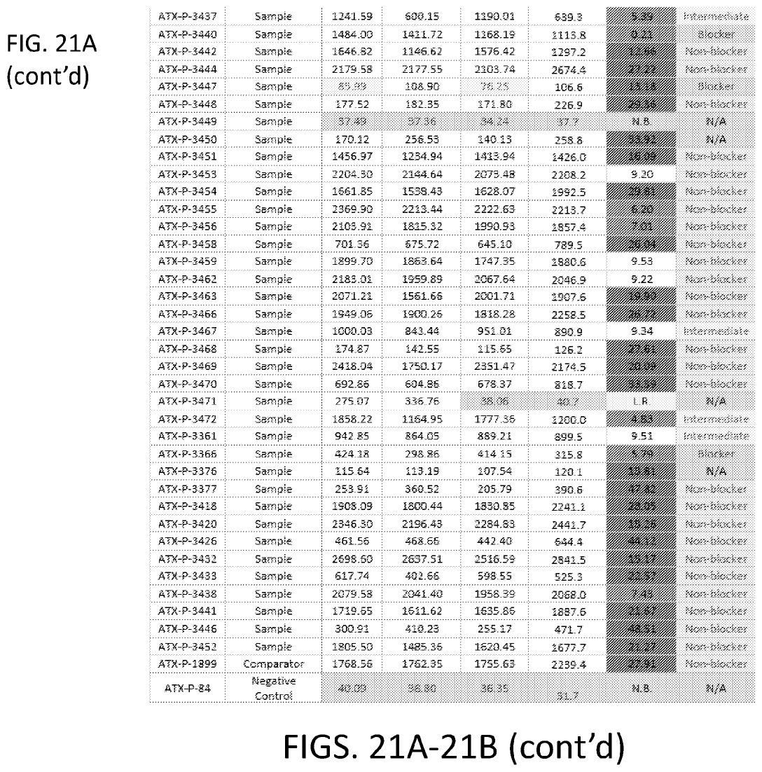

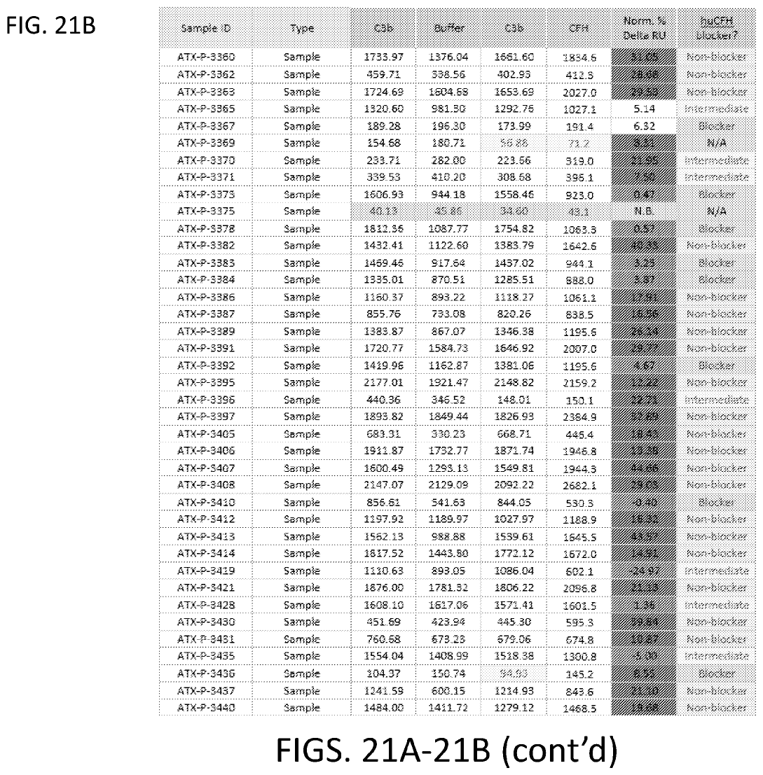

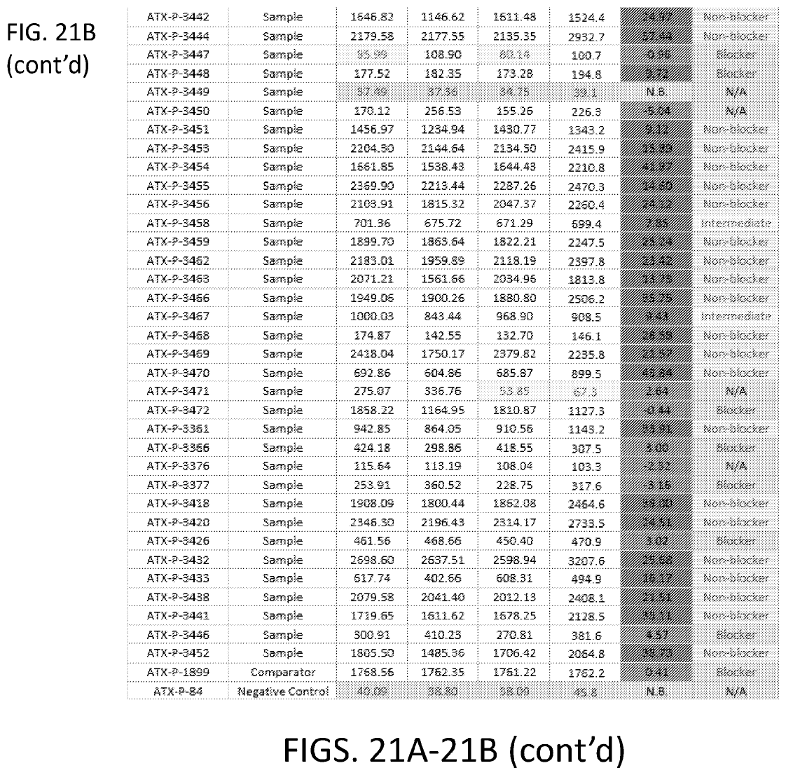

Additional experiments were conducted to determine the blocking potential of the C3 antibodies of the present disclosure for CFHR4 (FIG. 21B) and CFH (FIG. 21B).

### Sequences

The various amino acid sequences and nucleic acid sequences referenced herein are provided below.

TABLE 3
Anti-CFHR4 antibodies (CDR sequences)

| | | | |
|---|---|---|---|
| SEQ ID | Antibody | | |
| NO: | Name: | Descriptor: | Sequence: |

VH Family: IGHV1

| | | | |
|---|---|---|---|
| 1 | — | HCDR1 | X₁YX₂X₃X₄ |
| | | Consensus | (X₁ is S, T, G, or N; X₂ is G or Y; X₃ is I or M; X₄ is S, H, or Q) |
| 2 | ATX-P-591 | HCDR1 | SYGIS |
| 3 | ATX-P-583 | HCDR1 | TYGIS |
| 4 | ATX-P-602 | HCDR1 | GYYMH |
| 5 | ATX-P-601 | HCDR1 | SYYMH |
| 6 | ATX-P-607 | HCDR1 | SYYMH |
| 7 | ATX-P-597 | HCDR1 | NYYMQ |
| 8 | — | HCDR2 | X₁IX₂X₃X₄X₅GX₆TX₇X₈X₉X₁₀X₁₁X₁₂QX₁₃ |
| | | Consensus | (X₁ is W or I; X₂ is S, N, or D; X₃ is A or P; X₄ is Y, N, or S; X₅ is |
| | | | N, G, or S; X₆ is N, S, or G; X7 is N, H, T, or S; X₈ is Y or N; X₉ |
| | | | is A or Y; X₁₀ is Q or A; X₁₁ is K or S; X₁₂ is L or F; X₁₃ is G or |
| | | | D) |
| 9 | ATX-P-591 | HCDR2 | WISAYNGNTNYAQKLQG |
| 10 | ATX-P-583 | HCDR2 | WISAYSGNTHYAQSLQD |
| 11 | ATX-P-602 | HCDR2 | WINPNSGGTNYAKKFQG |
| 12 | ATX-P-601 | HCDR2 | IINPSGGSTNYAQKFQG |
| 13 | ATX-P-607 | HCDR2 | IINPSGGSTSYAQKFQG |
| 14 | ATX-P-597 | HCDR2 | IIDPSGGSTTYAQKFQG |
| 15 | ATX-P-591 | HCDR3 | DLRGTAFDY |
| 16 | ATX-P-583 | HCDR3 | DEGYCSSTGCLNLFDP |
| 17 | ATX-P-602 | HCDR3 | GDTIFRLVTPLDY |
| 18 | ATX-P-601 | HCDR3 | DQSGGMDV |
| 19 | ATX-P-607 | HCDR3 | DPEYSSAFDI |
| 20 | ATX-P-597 | HCDR3 | DEGTDRPGWGY |

VH Family: IGHV3

| | | | |
|---|---|---|---|
| 21 | — | HCDR1 | X₁YX₂X₃X₄ |
| | | Consensus | (X₁ is S, T, R, or D; X₂ is T, V, A, G, S, or E; X₃ is M or I; X₄ is |
| | | | N, S, or H) |
| 22 | ATX-P-564 | HCDR1 | SYTMN |
| 23 | ATX-P-568 | HCDR1 | SYVMS |
| 24 | ATX-P-574 | HCDR1 | SYAMS |
| 25 | ATX-P-565 | HCDR1 | TYGIH |
| 26 | ATX-P-578 | HCDR1 | RYSMN |
| 27 | ATX-P-588 | HCDR1 | DYAMH |
| 28 | ATX-P-600 | HCDR1 | DYAMH |
| 29 | ATX-P-569 | HCDR1 | DYAMH |
| 30 | ATX-P-596 | HCDR1 | DYAMH |
| 31 | ATX-P-576 | HCDR1 | DYEMH |
| 32 | — | HCDR2 | X₁IX₂X₃X₄X₅X₆X₇X₈X₉X₁₀X₁₁DX₁₂VX₁₃G |
| | | Consensus | (X₁ is S, V, Y, G or D; X₂ is S or W; X₃ is S, V, G, H, or W; X₄ is |
| | | | S, D, N or T; X₅ is S or G; X₆ is S or G; X₇ is Y, S, T or R; X₈ is |
| | | | T, K, I or V; X₉ is Y, F, N, or G; X₁₀ is Y or H; X₁₁ is A or V; X₁₂ |
| | | | is S or P; X₁₃ is R, K, or T) |
| 33 | ATX-P-564 | HCDR2 | SISSSSSYTYYADSVRG |
| 34 | ATX-P-568 | HCDR2 | VISVSGGSTFYADSVRG |
| 35 | ATX-P-574 | HCDR2 | VISGSGGTTYYADSVKG |
| 36 | ATX-P-565 | HCDR2 | VIWHDGSSKNYVDSVTG |
| 37 | ATX-P-578 | HCDR2 | YISSSSSTIFHADSVKG |
| 38 | ATX-P-588 | HCDR2 | GISWNSGSIGYADSVKG |
| 39 | ATX-P-600 | HCDR2 | GISWSGGSIGYADSVKG |
| 40 | ATX-P-569 | HCDR2 | GISWNSGSIGYADSVKG |
| 41 | ATX-P-596 | HCDR2 | GISWTGGSVGYADPVKG |
| 42 | ATX-P-576 | HCDR2 | DISRNSGRIGYADSVKG |
| 43 | ATX-P-564 | HCDR3 | SASIAAGMDV |
| 44 | ATX-P-568 | HCDR3 | DGLAVAGEDH |
| 45 | ATX-P-574 | HCDR3 | RGDYGDYMDV |
| 46 | ATX-P-565 | HCDR3 | RGNWNYGFYYYYMDV |
| 47 | ATX-P-578 | HCDR3 | GNWNYYYMDV |
| 48 | ATX-P-588 | HCDR3 | DRDDGYNYAGVFDY |
| 49 | ATX-P-600 | HCDR3 | DREDGYNYGGVEDY |
| 50 | ATX-P-569 | HCDR3 | DIGNGYTKGDVEDI |
| 51 | ATX-P-596 | HCDR3 | DMGDGYSRNWPFDY |
| 52 | ATX-P-576 | HCDR3 | DIWGSGYFDY |

VH Family: IGHV4

| | | | |
|---|---|---|---|
| 53 | — | HCDR1 | X₁X₂X₃WX₄X₅ |
| | | Consensus | (X₁ is T, S. G, or I; X₂ is S, R, Y, or H; X₃ is D, N, H, K or Y; X₄ |
| | | | is W or S; X₅ is T or S) |
| 54 | ATX-P-580 | HCDR1 | TSDWWT |
| 55 | ATX-P-579 | HCDR1 | TSNWWT |
| 56 | ATX-P-577 | HCDR1 | SSNWWT |
| 57 | ATX-P-608 | HCDR1 | SSNWWS |
| 58 | ATX-P-609 | HCDR1 | SSNWWS |
| 59 | ATX-P-603 | HCDR1 | GSHWWS |
| 60 | ATX-P-582 | HCDR1 | ISKWWS |
| 61 | ATX-P-598 | HCDR1 | SSNWWS |
| 62 | ATX-P-571 | HCDR1 | SRNWWS |
| 63 | ATX-P-570 | HCDR1 | GYYWS |
| 64 | ATX-P-563 | HCDR1 | SHYWS |
| 65 | — | HCDR2 | X₁X₂X₃X₄X₅GX₆X₇X₈X₉X₁₀PLSX₁₁S |
| | | Consensus | (X₁ is E or Y; X₂ is I, T, or V; X₃ is Y, H, or F; X₄ is H or Y; X₅ is |
| | | | S, D, T, G; X₆ is S, T, G, or N; X₇ is T or I; X₈ is N or K; X₉ is Y, |
| | | | K, or S; X₁₀ is N, S, K, or H; X₁₁ is K or Q) |
| 66 | ATX-P-580 | HCDR2 | EIYHSGSTNYNPSLKS |
| 67 | ATX-P-579 | HCDR2 | EIYHDGSTNYSPSLQS |
| 68 | ATX-P-577 | HCDR2 | ETYHDGSTNYNPSLKS |
| 69 | ATX-P-608 | HCDR2 | EIYHTGSTNKKPSLKS |
| 70 | ATX-P-609 | HCDR2 | EIYHSGSTNYNPSLKS |
| 71 | ATX-P-603 | HCDR2 | EIYHTGSTNKNPSLKS |
| 72 | ATX-P-582 | HCDR2 | EIYHTGSTNYHPSLKS |
| 73 | ATX-P-598 | HCDR2 | EIYHSGTTNYNPSLKS |
| 74 | ATX-P-571 | HCDR2 | EIYHGGTTNYNPSLKS |
| 75 | ATX-P-570 | HCDR2 | YVHYSGGTKSNPSLKS |
| 76 | ATX-P-563 | HCDR2 | YIFYSGNINYNPSLKS |
| 77 | ATX-P-580 | HCDR3 | WYFDL |
| 78 | ATX-P-579 | HCDR3 | FYIDV |
| 79 | ATX-P-577 | HCDR3 | FYFDY |
| 80 | ATX-P-608 | HCDR3 | DREGGTTEAFDI |
| 81 | ATX-P-609 | HCDR3 | DQLSSGPTDAFDI |
| 82 | ATX-P-603 | HCDR3 | DQYTGSPRDAFDI |
| 83 | ATX-P-582 | HCDR3 | DELRLGELSPSLDI |
| 84 | ATX-P-598 | HCDR3 | DRLRLGESSSDVFDI |
| 85 | ATX-P-571 | HCDR3 | GGLRLGELSPNAFDI |
| 86 | ATX-P-570 | HCDR3 | ERYEWNSYFDY |
| 87 | ATX-P-563 | HCDR3 | DRGGSYSYHFDC |

VH Family: IGHV6

| | | | |
|---|---|---|---|
| 88 | — | HCDR1 | SNX₁AX₂WN |
| | | Consensus | (X₁ is S, T, or N; X₂ is A or S) |
| 89 | ATX-P-566 | HCDR1 | SNSAAWN |
| 90 | ATX-P-594 | HCDR1 | SASASWN |
| 91 | ATX-P-604 | HCDR1 | SNSAAWN |
| 92 | ATX-P-595 | HCDR1 | SNSAAWN |
| 93 | ATX-P-610 | HCDR1 | SNSAAWN |
| 94 | ATX-P-572 | HCDR1 | SNSAAWN |
| 95 | ATX-P-587 | HCDR1 | SNSAAWN |
| 96 | ATX-P-592 | HCDR1 | SNSAAWN |
| 97 | ATX-P-560 | HCDR1 | SNTAAWN |
| 98 | ATX-P-581 | HCDR1 | SNNAAWN |
| 99 | ATX-P-573 | HCDR1 | SNSAAWN |
| 100 | ATX-P-561 | HCDR1 | SNSAAWN |
| 101 | ATX-P-562 | HCDR1 | SNSAAWN |
| 102 | — | HCDR2 | X₁TX₂YRSX₃X₄X₅X₆X₇X₈X₉X₁₀SX₁₁X₁₂S |
| | | Consensus | (X₁ is R, T, M, or K; X₂ is Y or F; X₃ is K or R; X₄ is W or L; X₅ |
| | | | is F, Y, or S; X₆ is N, S, D, or A; X₇ is N, V, G, D, Y, or A; X₈ is |
| | | | Y or F; X₉ is S, A, or P; X₁₀ is V, L, or A; X₁₁ is Vor M; X₁₂ is K |
| | | | or S) |
| 103 | ATX-P-566 | HCDR2 | RTYYRSKWFNNYSVSVKS |
| 104 | ATX-P-594 | HCDR2 | TTYYRSKWFNVYAVSVKS |
| 105 | ATX-P-604 | HCDR2 | RTYYRSKWFNGYAVSVKS |
| 106 | ATX-P-595 | HCDR2 | RTYYRSKWENDYALSVKS |
| 107 | ATX-P-610 | HCDR2 | RTYYRSKWYNDYALSVKS |
| 108 | ATX-P-572 | HCDR2 | MTYYRSKWYSVFAVSVKS |
| 109 | ATX-P-587 | HCDR2 | KTYYRSKWYNYYALSVKS |
| 110 | ATX-P-592 | HCDR2 | RTYYRSKWYSDYPLSVKS |
| 111 | ATX-P-560 | HCDR2 | RTYYRSKWYNDYAVSVKS |
| 112 | ATX-P-581 | HCDR2 | RTYYRSKWYNAYAVSVKS |
| 113 | ATX-P-573 | HCDR2 | RTYYRSKLYSDYAVSVKS |
| 114 | ATX-P-561 | HCDR2 | RTYYRSKWYNGYAVSMKS |
| 115 | ATX-P-562 | HCDR2 | RTFYRSRWSAAYAASVSS |
| 116 | ATX-P-566 | HCDR3 | DLGELLDWYFDL |
| 117 | ATX-P-594 | HCDR3 | DRGDLLHWYFDL |
| 118 | ATX-P-604 | HCDR3 | DRGELLHWYFDL |
| 119 | ATX-P-595 | HCDR3 | EPGIVAPGPEDY |
| 120 | ATX-P-610 | HCDR3 | EPGIAAPGPFDY |
| 121 | ATX-P-572 | HCDR3 | EGGIVGATPFDY |
| 122 | ATX-P-587 | HCDR3 | DGDYYLPSPFDL |
| 123 | ATX-P-592 | HCDR3 | DSGIVETTPFDY |
| 124 | ATX-P-560 | HCDR3 | DNWNNGGPGFDP |
| 125 | ATX-P-581 | HCDR3 | EGGYIYGLDQ |
| 126 | ATX-P-573 | HCDR3 | EGGYTYGYDY |
| 127 | ATX-P-561 | HCDR3 | EGVGATTGFDY |
| 128 | ATX-P-562 | HCDR3 | EGVGSSTGFDY |

VL Family: IGKV1

| | | | |
|---|---|---|---|
| 129 | - | LCDR1 | RX₁SX₂X₃X₄X₅X₆X₇LX₈ |
| | | Consensus | (X₁ is A or T; X₂ is Q or K; X₃ is G, S, D, or N; X₄ is I, F, or V; X₅ |
| | | | is T, R, A, S, N, G, or I; X₆ is T, N, G, S, I, K, or Y; X₇ is W, D, or |
| | | | Y; X₈ is A, T, G, N, or D) |
| 130 | ATX-P-574 | LCDR1 | RASQGITTWLA |
| 131 | ATX-P-576 | LCDR1 | RTSQGIRNWLA |
| 132 | ATX-P-563 | LCDR1 | RASQSFAGWLA |
| 133 | ATX-P-610 | LCDR1 | RASQGISSWLA |
| 134 | ATX-P-560 | LCDR1 | RASQGISSWLA |
| 135 | ATX-P-581 | LCDR1 | RASQGVNNWLA |
| 136 | ATX-P-573 | LCDR1 | RASQGISSWLA |
| 137 | ATX-P-561 | LCDR1 | RASQGISSWLT |
| 138 | ATX-P-562 | LCDR1 | RASKGISIWLA |
| 139 | ATX-P-580 | LCDR1 | RASQGIRNDLG |
| 140 | ATX-P-579 | LCDR1 | RASQGIRKDLG |
| 141 | ATX-P-577 | LCDR1 | RASQDIRNDLG |
| 142 | ATX-P-591 | LCDR1 | RASQSINTYLN |
| 143 | ATX-P-583 | LCDR1 | RASQNIGSYLN |
| 144 | ATX-P-601 | LCDR1 | RASQSINYLN |
| 145 | ATX-P-607 | LCDR1 | RASQSIRSYLN |
| 146 | ATX-P-597 | LCDR1 | RASQSISSYLD |
| 147 | ATX-P-582 | LCDR1 | RASQNINNYLN |
| 148 | ATX-P-571 | LCDR1 | RASQSISSYLN |
| 149 | ATX-P-568 | LCDR1 | RASQSISTWLA |
| 150 | ATX-P-578 | LCDR1 | RASQSVNSWLA |
| 151 | ATX-P-569 | LCDR1 | RASQNIITWLA |
| 152 | — | LCDR2 | X₁X₂SX₃LX₄X₅ |
| | | Consensus | (X₁ is G, A, T, or K; X₂ is A or T; X₃ is S, T, G, or N; X₄ is E, Q, |
| | | | or L; X₅ is S, T, or G) |
| 153 | ATX-P-574 | LCDR2 | GASSLES |
| 154 | ATX-P-576 | LCDR2 | AASSLQT |
| 155 | ATX-P-563 | LCDR2 | AASTLQS |
| 156 | ATX-P-610 | LCDR2 | AASSLQS |
| 157 | ATX-P-560 | LCDR2 | AASSLQS |
| 158 | ATX-P-581 | LCDR2 | AASSLLS |
| 159 | ATX-P-573 | LCDR2 | AASSLQS |
| 160 | ATX-P-561 | LCDR2 | AASSLQG |
| 161 | ATX-P-562 | LCDR2 | AASSLQS |
| 162 | ATX-P-580 | LCDR2 | AASSLQS |
| 163 | ATX-P-579 | LCDR2 | TASTLQS |
| 164 | ATX-P-577 | LCDR2 | AASSLQS |
| 165 | ATX-P-591 | LCDR2 | AASSLQS |
| 166 | ATX-P-583 | LCDR2 | ATSTLQS |
| 167 | ATX-P-601 | LCDR2 | AASSLQS |
| 168 | ATX-P-607 | LCDR2 | AASSLQS |
| 169 | ATX-P-597 | LCDR2 | AASSLQS |
| 170 | ATX-P-582 | LCDR2 | AASSLQS |
| 171 | ATX-P-571 | LCDR2 | AASSLQS |
| 172 | ATX-P-568 | LCDR2 | KASGLES |
| 173 | ATX-P-578 | LCDR2 | KASNLEG |
| 174 | ATX-P-569 | LCDR2 | KASSLES |
| 175 | ATX-P-574 | LCDR3 | QQANSFPWT |
| 176 | ATX-P-576 | LCDR3 | QQANSFPYT |
| 177 | ATX-P-563 | LCDR3 | QQANSFPLT |
| 178 | ATX-P-610 | LCDR3 | QQNYRTPIT |
| 179 | ATX-P-560 | LCDR3 | QQANSFPPT |
| 180 | ATX-P-581 | LCDR3 | QQANSFPIT |
| 181 | ATX-P-573 | LCDR3 | QQANSFPPT |
| 182 | ATX-P-561 | LCDR3 | QQANSFPPT |
| 183 | ATX-P-562 | LCDR3 | QQANSFPPT |
| 184 | ATX-P-580 | LCDR3 | LHFNSYPFT |
| 185 | ATX-P-579 | LCDR3 | LQYNNYPFT |
| 186 | ATX-P-577 | LCDR3 | LQYNNYPFT |
| 187 | ATX-P-591 | LCDR3 | QHSFTTPFT |
| 188 | ATX-P-583 | LCDR3 | QQSFIIPFT |
| 189 | ATX-P-601 | LCDR3 | QQSYSTPIT |
| 190 | ATX-P-607 | LCDR3 | QQSYRTPVT |
| 191 | ATX-P-597 | LCDR3 | QQSYSTPLT |
| 192 | ATX-P-582 | LCDR3 | QQSYSTPLT |
| 193 | ATX-P-571 | LCDR3 | QETYSVPLT |
| 194 | ATX-P-568 | LCDR3 | QQYNSFSLI |
| 195 | ATX-P-578 | LCDR3 | QQYDTSWT |
| 196 | ATX-P-569 | LCDR3 | QQYSSYSYT |

VL Family: IGKV2

| | | | |
|---|---|---|---|
| 197 | — | LCDR1 | RSSQX₁LLHSX₂GYNX₃LD |
| | | Consensus | (X₁ is S or R; X₂ is T or S; X₃ is F or Y) |
| 198 | ATX-P-564 | LCDR1 | RSSQS LLHST GYNFLD |
| 199 | ATX-P-565 | LCDR1 | RSSQR LLHSS GYNYLD |
| 200 | — | LCDR2 | LX₁SX₂RAS |
| | | Consensus | (X₁ is A or G; X₂ is N or S) |
| 201 | ATX-P-564 | LCDR2 | LASNRAS |
| 202 | ATX-P-565 | LCDR2 | LGSSRAS |
| 203 | — | LCDR3 | MQX₁LQTPX₂T |
| | | Consensus | (X₁ is A or G; X₂ is Y or P) |
| 204 | ATX-P-564 | LCDR3 | MQALQTPYT |
| 205 | ATX-P-565 | LCDR3 | MQGLQTPPT |

VL Family: IGKV3

| | | | |
|---|---|---|---|
| 206 | — | LCDR1 | RASQX₁X₂X₃X₄X₅X₆X₇X₈A |
| | | Consensus | (X₁ is S, N, or T; X₂ is V or I; X₃ is S or R; X₄ is S, G or N; X₅ is |
| | | | Nor S; X₆ is L or Y; X₇ is A, L, or V) |
| 207 | ATX-P-608 | LCDR1 | RASQSVSSNLA |
| 208 | ATX-P-609 | LCDR1 | RASQSVSSNLA |
| 209 | ATX-P-602 | LCDR1 | RASQSVSSSYLA |
| 210 | ATX-P-588 | LCDR1 | RASQSVSSNLA |
| 211 | ATX-P-600 | LCDR1 | RASQNVRGSYLA |
| 212 | ATX-P-603 | LCDR1 | RASQSVSSSYLA |
| 213 | ATX-P-566 | LCDR1 | RASQSVRNSYVA |
| 214 | ATX-P-594 | LCDR1 | RASQSVSSSYLA |
| 215 | ATX-P-604 | LCDR1 | RASQTIRSSYLA |
| 216 | - | LCDR2 | GASX₁RAT |
| | | Consensus | (X₁ is T, S, or N) |
| 217 | ATX-P-608 | LCDR2 | GASTRAT |
| 218 | ATX-P-609 | LCDR2 | GASTRAT |
| 219 | ATX-P-602 | LCDR2 | GASSRAT |
| 220 | ATX-P-588 | LCDR2 | GASSRAT |
| 221 | ATX-P-600 | LCDR2 | GASNRAT |
| 222 | ATX-P-603 | LCDR2 | GASSRAT |
| 223 | ATX-P-566 | LCDR2 | GASSRAT |
| 224 | ATX-P-594 | LCDR2 | GASSRAT |
| 225 | ATX-P-604 | LCDR2 | GASSRAT |
| 226 | — | LCDR3 | QX₁YX₂X₃X₄X₅X₆T |
| | | Consensus | (X₁ is Q or H; X₂ is D or G; X₃ is N, S, or R; X₄ is W or S; X₅ is |
| | | | R, P, F, Y, V, or I; X₆ is T, W, L or I) |
| 227 | ATX-P-608 | LCDR3 | QQYD NWRT |
| 228 | ATX-P-609 | LCDR3 | QQYD NWRT |
| 229 | ATX-P-602 | LCDR3 | QQYG SSPWT |
| 230 | ATX-P-588 | LCDR3 | QQYG SSFT |
| 231 | ATX-P-600 | LCDR3 | QQYG SSYT |
| 232 | ATX-P-603 | LCDR3 | QHYG NSPLT |
| 233 | ATX-P-566 | LCDR3 | QQYG NSPLT |
| 234 | ATX-P-594 | LCDR3 | QQYG RSVT |
| 235 | ATX-P-604 | LCDR3 | QQYG RSII |

VL Family: IGKV4

| | | | |
|---|---|---|---|
| 236 | — | LCDR1 | KSSQSVLYSSNNKNYLA |
| | | Consensus | |
| 237 | ATX-P-596 | LCDR1 | KSSQSVLYSSNNKNYLA |
| 238 | ATX-P-598 | LCDR1 | KSSQSVLYSSNNKNYLA |
| 239 | ATX-P-570 | LCDR1 | KSSQSVLYSSNNKNYLA |
| 240 | ATX-P-595 | LCDR1 | KSSQSVLYSSNNKNYLA |
| 241 | ATX-P-572 | LCDR1 | KSSQSVLYSSNNKNYLA |
| 242 | ATX-P-587 | LCDR1 | KSSQSVLYSSNNKNYLA |
| 243 | ATX-P-592 | LCDR1 | KSSQSVLYSSNNKNYLA |
| 244 | — | LCDR2 | WASX₁RES |
| | | Consensus | (X₁ is T, P or N) |
| 245 | ATX-P-596 | LCDR2 | WASTRES |
| 246 | ATX-P-598 | LCDR2 | WASTRES |
| 247 | ATX-P-570 | LCDR2 | WASTRES |
| 248 | ATX-P-595 | LCDR2 | WASTRES |
| 249 | ATX-P-572 | LCDR2 | WASPRES |
| 250 | ATX-P-587 | LCDR2 | WASTRES |
| 251 | ATX-P-592 | LCDR2 | WASNRES |
| 252 | — | LCDR3 | QQX₁X₂X₃X₄PX₅X₆T |
| | | Consensus | (X₁ is Y or F; X₂ is G or Y; X₃ is S or N; X₄ is S, T, or I; X₅ is M, |
| | | | Y, or R; X₆ is Y or T) |
| 253 | ATX-P-596 | LCDR3 | QQYGSSPMYT |
| 254 | ATX-P-598 | LCDR3 | QQFYNTPYT |
| 255 | ATX-P-570 | LCDR3 | QQYYSSPRT |
| 256 | ATX-P-595 | LCDR3 | QQYYSTPYT |
| 257 | ATX-P-572 | LCDR3 | QQYYSIPRT |
| 258 | ATX-P-587 | LCDR3 | QQYYSTPYT |
| 259 | ATX-P-592 | LCDR3 | QQYYSTPYT |

TABLE 4
Anti-CFHR4 antibodies (VH and VL sequences)

| SEQ ID | Antibody | | |
|---|---|---|---|
| NO: | Name: | Descriptor: | Sequence: |
| 260 | ATX-P-583 | VH | QVQLVQSGAEVKKPGASVKVSCKASGYTFTTYGISWVRQAPGQ |
| | | | GLEWMGWISAYSGNTHYAQSLQDRVTMTTDTSTSTAYMEVRSL |
| | | | RSDDTAVYYCAKDEGYCSSTGCLNLFDPWGQGTLVTVSS |
| 261 | ATX-P-591 | VH | QVQLVQSGAEVKKPGASVKVSCKASGYTFTSYGISWVRQAPGQ |
| | | | GLEWMGWISAYNGNTNYAQKLQGRVTMTTDTSTSTAYMELRSL |
| | | | RSDDTAIYYCTRDLRGTAFDYWGQGTPVTVSS |
| 262 | ATX-P-602 | VH | QVQLVQSGAEVKKPGASVKVSCKASGYTFTGYYMHWVRQAPG |
| | | | QGLEWMGWINPNSGGTNYAKKFQGRVTMTRDTSISTAYMELSR |
| | | | LRSDDTAVYYCARGDTIFRLVTPLDYWGQGTLVTVSS |
| 263 | ATX-P-597 | VH | QVQLVQSGAEVKKPGASVKVSCKASGYTFTNYYMQWVRQAPG |
| | | | QGLEWLGIIDPSGGSTTYAQKFQGRVTMTRDTSTSTVHMELRSLR |
| | | | SEDTAVYYCARDEGTDRPGWGYWGQGTLVTVSS |
| 264 | ATX-P-601 | VH | QVQLVQSGAEVKKPGASVKVSCKASGYTFTSYYMHWVRQAPG |
| | | | QGLEWMGIINPSGGSTNYAQKFQGRLTMTRDTSTNTVYMELSSL |
| | | | RSEDTAVYYCATDQSGGMDVWGKGTTVTVSS |
| 265 | ATX-P-607 | VH | QVQLVQSGAEVKKPGASVKVSCKASGYTFTSYYMHWVRQAPG |
| | | | QGLEWMGIINPSGGSTSYAQKFQGRVTMTRDTSTNTVYMELSSL |
| | | | RSEDTAVYYCARDPEYSSAFDIWGQGTMVTVSS |
| 266 | ATX-P-583 | VH - nucleotide | CAGGTTCAGCTGGTGCAGTCTGGAGCTGAGGTGAAGAAGCCT |
| | | | GGGGCCTCAGTGAAGGTCTCCTGCAAGGCTTCTGGTTACACCT |
| | | | TTACCACCTATGGTATCAGCTGGGTGCGACAGGCCCCTGGACA |
| | | | AGGGCTTGAGTGGATGGGATGGATCAGCGCTTACAGTGGTAA |
| | | | CACACACTATGCACAGAGCCTCCAGGACAGAGTCACCATGAC |
| | | | CACAGACACATCCACGAGTACAGCCTACATGGAGGTGAGGAG |
| | | | CCTGAGATCTGACGACACGGCCGTGTATTACTGTGCGAAAGAT |
| | | | GAGGGATATTGTAGTAGTACCGGCTGCTTAAACTTGTTCGACC |
| | | | CCTGGGGCCAGGGAACCCTGGTCACCGTCTCCTCA |
| 267 | ATX-P-591 | VH - nucleotide | CAGGTTCAGCTGGTGCAGTCTGGAGCTGAGGTGAAGAAGCCT |
| | | | GGGGCCTCAGTGAAGGTCTCCTGCAAGGCTTCTGGTTACACCT |
| | | | TTACCAGCTATGGTATCAGCTGGGTGCGACAGGCCCCTGGACA |
| | | | AGGGCTTGAGTGGATGGGATGGATCAGCGCTTACAATGGTAA |
| | | | CACAAACTATGCACAGAAGCTCCAGGGCAGAGTCACCATGAC |
| | | | CACAGACACATCCACGAGCACAGCCTACATGGAGCTGAGGAG |
| | | | CCTGAGATCTGACGACACTGCCATTTATTACTGTACGAGAGAT |
| | | | CTACGTGGAACTGCCTTTGACTACTGGGGCCAGGGAACCCCGG |
| | | | TCACTGTCTCCTCA |
| 268 | ATX-P-602 | VH - nucleotide | CAGGTGCAGCTGGTGCAATCTGGGGCTGAGGTGAAGAAGCCT |
| | | | GGGGCCTCAGTGAAGGTCTCCTGCAAGGCTTCTGGATACACCT |
| | | | TCACCGGCTACTATATGCACTGGGTGCGACAGGCCCCTGGACA |
| | | | AGGACTTGAGTGGATGGGATGGATCAACCCTAACAGTGGTGG |
| | | | CACAAACTATGCAAAGAAGTTTCAGGGCAGGGTCACCATGAC |
| | | | CAGGGACACGTCCATCAGCACAGCCTACATGGAGTTGAGCAG |
| | | | GCTGAGATCTGACGACACGGCCGTGTATTACTGTGCGAGAGG |
| | | | GGATACGATTTTTCGACTGGTTACCCCTCTTGACTACTGGGGC |
| | | | CAGGGAACCCTGGTCACCGTCTCCTCA |
| 269 | ATX-P-597 | VH - nucleotide | CAGGTGCAGCTGGTGCAATCTGGAGCTGAGGTGAAGAAGCCT |
| | | | GGGGCCTCAGTGAAGGTTTCCTGCAAGGCATCTGGATACACCT |
| | | | TCACCAACTACTATATGCAGTGGGTGCGACAGGCCCCTGGACA |
| | | | AGGGCTTGAGTGGCTGGGAATAATCGACCCTAGTGGTGGTAG |
| | | | CACAACCTATGCACAGAAGTTCCAGGGCAGAGTCACCATGAC |
| | | | CAGGGACACGTCCACGAGCACAGTCCACATGGAGCTGAGAAG |
| | | | TCTGAGATCTGAGGACACGGCCGTCTATTATTGCGCGAGAGAT |
| | | | GAGGGAACAGATCGTCCCGGGTGGGGCTACTGGGGCCAGGGA |
| | | | ACCCTGGTCACTGTCTCCTCA |
| 270 | ATX-P-601 | VH - nucleotide | CAGGTGCAGCTGGTGCAATCTGGGGCTGAGGTGAAGAAGCCT |
| | | | GGGGCCTCAGTGAAGGTTTCCTGCAAGGCATCTGGATACACCT |
| | | | TCACCAGCTACTATATGCACTGGGTGCGACAGGCCCCTGGACA |
| | | | AGGGCTTGAGTGGATGGGAATAATCAACCCTAGTGGTGGTAG |
| | | | CACAAACTACGCACAGAAGTTCCAGGGCAGACTCACCATGAC |
| | | | CAGGGACACGTCCACGAACACAGTCTACATGGAACTGAGTAG |
| | | | CCTGAGATCTGAGGACACGGCCGTGTATTACTGTGCGACAGAT |
| | | | CAAAGTGGGGGGATGGACGTCTGGGGCAAAGGGACCACGGTC |
| | | | ACCGTCTCCTCA |
| 271 | ATX-P-607 | VH - nucleotide | CAGGTGCAGCTGGTGCAATCTGGGGCTGAGGTGAAGAAGCCT |
| | | | GGGGCCTCAGTGAAGGTTTCCTGCAAGGCATCTGGATACACCT |
| | | | TCACCAGTTACTATATGCACTGGGTGCGACAGGCCCCTGGACA |
| | | | AGGGCTTGAGTGGATGGGAATAATCAACCCTAGTGGTGGTAG |
| | | | CACAAGCTACGCACAGAAGTTCCAGGGCAGAGTCACCATGAC |
| | | | CAGGGACACGTCAACGAACACAGTCTACATGGAGCTGAGCAG |
| | | | CCTTCGATCTGAGGACACGGCCGTGTATTACTGTGCGAGAGAT |
| | | | CCCGAGTATAGCAGTGCTTTTGATATCTGGGGACAAGGGACAA |
| | | | TGGTCACCGTCTCCTCA |
| 272 | ATX-P-583 | VL | DIQMTQSPSSLSASVGDRVTITCRASQNIGSYLNWYQQKPGNAPK |
| | | | LLIYATSTLQSGVPSRFSGSGSGTDFTLUISSLHPEDFTVYYCQQSFI |
| | | | IPFTFGPGTKVDIK |
| 273 | ATX-P-591 | VL | DIQMTQSPSSLSASVGDRVTITCRASQSINTYLNWYQQKPGKAPK |
| | | | LLIYAASSLQSGVPSRFSGSGSGTDFTLTISSLQPEDFATYSCQHSF |
| | | | TTPFTFGPGTKLEIK |
| 274 | ATX-P-602 | VL | EIVLTQSPGTLSLSPGERATLSCRASQSVSSSYLAWYQQKPGQAP |
| | | | RLLIYGASSRATGIPDRFSGSGSGTQFTLTISSLQSEDFAVYYCQQ |
| | | | YGSSPWTFGQGTKVDIK |
| 275 | ATX-P-597 | VL | DIQMTQSPSSLSASIGDRVTITCRASQSISSYLDWYQRKSGKAPKL |
| | | | LIYAASSLQSGVPSRFSGSGSGTDFTLTISSLQPEDFATYYCQQSYS |
| | | | TPLTFGGGTKVDIK |
| 276 | ATX-P-601 | VL | DIQMTQSPSSLSASVGDRVTITCRASQSINIYLNWYQQKPEKDPKL |
| | | | LIYAASSLQSGVPSRFSGSGSGTDFTLTISNLQPEDFATYYCQQSY |
| | | | STPITFGQGTRLEIK |
| 277 | ATX-P-607 | VL | DIQMTQSPSSLSASVGDRGTITCRASQSIRSYLNWYQQKPGKAPK |
| | | | LLIYAASSLQSGVPSRFSGSGSGTDFTLTISSLQPEDFATYYCQQSY |
| | | | RTPVTFGPGTKVDIK |
| 278 | ATX-P-583 | VL - nucleotide | GACATCCAGATGACCCAGTCTCCGTCCTCCCTGTCTGCATCTGT |
| | | | AGGAGACAGAGTCACCATCACTTGCCGGGCAAGTCAGAACAT |
| | | | TGGCAGTTATTTAAATTGGTATCAGCAGAAACCAGGGAATGCC |
| | | | CCTAAACTCCTGATCTATGCTACATCCACTTTGCAAAGTGGGG |
| | | | TCCCATCAAGGTTCAGTGGCAGTGGATCTGGGACAGATTTCAC |
| | | | TCTCATCATCAGCAGTCTGCACCCTGAAGATTTTACAGTTTATT |
| | | | ACTGTCAACAGAGTTTCATTATCCCATTCACTTTCGGCCCTGGG |
| | | | ACCAAAGTGGATATCAAA |
| 279 | ATX-P-591 | VL - nucleotide | GACATCCAGATGACTCAGTCTCCATCCTCCCTGTCTGCATCTGT |
| | | | TGGAGACAGAGTCACCATCACTTGCCGGGCAAGTCAGAGCAT |
| | | | TAACACCTATTTAAATTGGTATCAGCAGAAACCAGGGAAAGC |
| | | | CCCTAAGCTCCTGATCTATGCTGCATCCAGTTTGCAAAGTGGG |
| | | | GTCCCATCAAGGTTCAGTGGCAGTGGATCTGGGACAGATTTCA |
| | | | CTCTCACCATCAGCAGTCTGCAACCTGAAGATTTTGCAACTTA |
| | | | CTCCTGTCAACATAGTTTCACTACCCCATTCACTTTCGGCCCTG |
| | | | GGACCAAGCTGGAAATCAAA |
| 280 | ATX-P-602 | VL - nucleotide | GAAATTGTGTTGACGCAGTCTCCAGGCACCCTGTCTTTGTCTCC |
| | | | AGGGGAAAGAGCCACCCTCTCCTGCAGGGCCAGTCAGAGTGT |
| | | | TAGCAGCAGCTACTTAGCCTGGTACCAGCAGAAACCTGGCCA |
| | | | GGCTCCCAGGCTCCTCATCTATGGTGCATCCAGTAGGGCCACT |
| | | | GGCATCCCAGACAGGTTCAGTGGCAGTGGGTCTGGGACACAG |
| | | | TTCACTCTCACCATCAGCAGCCTGCAGTCTGAAGATTTTGCAG |
| | | | TTTATTACTGTCAGCAGTATGGTAGCTCACCGTGGACGTTCGG |
| | | | CCAAGGGACCAAAGTGGATATCAAA |
| 281 | ATX-P-597 | VL - nucleotide | GACATCCAGATGACCCAGTCTCCATCCTCCCTGTCTGCATCTAT |
| | | | TGGAGACAGAGTCACCATCACTTGCCGGGCAAGTCAGAGCAT |
| | | | TAGCAGCTATTTAGATTGGTATCAGCGGAAATCAGGGAAAGC |
| | | | CCCTAAACTCCTGATCTATGCTGCATCCAGTTTGCAAAGTGGG |
| | | | GTCCCATCAAGGTTCAGTGGCAGTGGATCTGGGACAGATTTCA |
| | | | CTCTCACCATCAGCAGTCTGCAACCTGAAGATTTTGCAACTTA |
| | | | CTACTGTCAACAGAGTTACAGTACCCCGCTCACTTTCGGQGGA |
| | | | GGGACCAAAGTGGATATCAAA |
| 282 | ATX-P-601 | VL - nucleotide | GACATCCAGATGACCCAGTCTCCATCCTCCCTGTCTGCATCTGT |
| | | | AGGAGACAGAGTCACCATCACTTGCCGGGCAAGTCAGAGCAT |
| | | | TAATATCTATTTAAATTGGTATCAGCAGAAACCAGAGAAAGAC |
| | | | CCTAAGCTCCTGATCTATGCTGCGTCCAGTTTGCAAAGTGGGG |
| | | | TCCCATCAAGGTTCAGTGGCAGTGGATCTGGGACAGATTTCAC |
| | | | TCTCACCATCAGCAATCTGCAACCTGAAGATTTTGCAACTTAC |
| | | | TACTGTCAACAGAGTTACAGTACCCCGATCACCTTCGGCCAAG |
| | | | GGACACGACTGGAGATTAAA |
| 283 | ATX-P-607 | VL - nucleotide | GACATCCAGATGACACAGTCTCCATCCTCCCTGTCTGCATCTG |
| | | | TAGGAGACAGAGGCACCATCACTTGCCGGGCAAGTCAGAGCA |
| | | | TTAGAAGTTATTTAAATTGGTATCAGCAGAAACCAGGGAAAG |
| | | | CCCCTAAACTCCTGATCTATGCTGCATCCAGTTTGCAAAGTGG |
| | | | GGTCCCATCAAGGTTCAGTGGCAGTGGATCTGGGACAGATTTC |
| | | | ACTCTCACCATCAGCAGTCTGCAACCTGAAGATTTTGCAACTT |
| | | | ACTACTGTCAACAGAGTTACAGAACCCCAGTCACTTTCGGCCC |
| | | | TGGGACCAAAGTGGATATCAAA |
| 284 | ATX-P-564 | VH | EVQLVESGGGLVKPGGSLRLSCAASGFTFSSYTMNWVRQAPGKG |
| | | | LEWVSSISSSSSYTYYADSVRGRFTISRDNAKNSLYLQMSSLRAE |
| | | | DTAVYYCARSASIAAGMDVWGKGTTVTVSS |
| 285 | ATX-P-568 | VH | EVQLVESGGGLVQPGGSLRLSCAASGFPFSSYVMSWVRQAPGKG |
| | | | LEWVSVISVSGGSTFYADSVRGRFTISRDNSKNTLYLQVYSLRAE |
| | | | DTAVYYCAKDGLAVAGFDHWGQGTLVTVSS |
| 286 | ATX-P-574 | VH | EVQLVESGGGLVQRGGSLRLSCVASGFTFSSYAMSWVRQAPGKG |
| | | | LEWVSVISGSGGTTYYADSVKGRFTISRDDSKKTL YLQMNSLRAE |
| | | | DTAVYYCAKRGDYGDYMDVWGKGTTVTVSS |
| 287 | ATX-P-565 | VH | QVQLVESGGGVVQPGRSLRLSCAASGFTFNTYGIHWVRQAPGKG |
| | | | LEWVAVIWHDGSSKNYVDSVTGRFTISRDNSKNTVYLQMNSLRA |
| | | | EDTAVYYCARRGNWNYGFYYYYMDVWGKGTTVTVSS |
| 288 | ATX-P-578 | VH | EVQLVESGGGLVQPGGSLRLSCAASGFTFSRYSMNWVRQAPGKG |
| | | | LEWVSYISSSSSTIFHADSVKGRFTISRDNAKNSLYLQMNSLRDED |
| | | | TAVYYCARGNWNYYYMDVWGKGTTVTVSS |
| 289 | ATX-P-569 | VH | EVQLVESGGGLAQFGRSLRLSCAASGFTFDDYAMHWLRQAPGM |
| | | | GLEWVSGISWNSGSIGYADSVKGRFTIARDNAKNSVHLQMNSLR |
| | | | AEDTALYYCAKDIGNGYTKGDVFDIWGQGTMVTVSS |
| 290 | ATX-P-576 | VH | EVQLVESGGGLVQPGRSLRLSCTASGFTEDDYEMHWVRQAPGK |
| | | | GLEWVSDISRNSGRIGYADSVKGRFTISRDNAKNSLYLQMNSLRA |
| | | | EDTALYYCAKDIWGSGYFDYWGQGILVTVSS |
| 291 | ATX-P-588 | VH | EVQLVESGGGLVQPGRSLRLSCAASGFTFEDYAMHWVRQAPGK |
| | | | GLEWVSGISWNSGSIGYADSVKGRFTISRDNAKKSLYLQMNSLR |
| | | | AEDTALYYCTKDRDDGYNYAGVFDYWGQGTLVTVSS |
| 292 | ATX-P-596 | VH | EVQLVESGGGLVQPGRSLRLSCAASGFSEDDYAMHWVRQVPGK |
| | | | GLEWVSGISWTGGSVGYADPVKGRFTISRDNAKNSLYLQMNSLR |
| | | | PEDTALYYCAKDMGDGYSRNWPFDYWGQGTLVTVSS |
| 293 | ATX-P-600 | VH | EVQLVESGGGLVQPGRSLRLSCAASGFTFDDYAMHWVRQVPGR |
| | | | GLEWVSGISWSGGSIGYADSVKGRFTISRDNSKNSLYLQMNSLRA |
| | | | EDTALYYCTKDREDGYNYGGVFDYWGQGTLVTVSS |
| 294 | ATX-P-564 | VH - nucleotide | GAGGTGCAGCTGGTGGAGTCTGGGGGAGGCCTGGTCAAGCCT |
| | | | GGGGGGTCCCTGAGACTCTCCTGTGCAGCCTCTGGATTCACCT |
| | | | TCAGTAGCTATACCATGAACTGGGTCCGCCAGGCTCCAGGGAA |
| | | | GGGGCTGGAGTGGGTGTCATCCATTAGTAGTAGTAGTAGTTAC |
| | | | ACATACTACGCAGACTCAGTGAGGGGCCGATTCACCATCTCCA |
| | | | GAGACAACGCCAAGAACTCACTGTATCTGCAAATGAGCAGCC |
| | | | TGAGAGCCGAGGACACAGCTGTGTATTACTGTGCGAGATCGG |
| | | | CGAGTATAGCAGCTGGGATGGACGTCTGGGGCAAAGGGACCA |
| | | | CGGTCACCGTCTCCTCA |
| 295 | ATX-P-568 | VH - nucleotide | GAGGTGCAGCTGGTGGAGTCTGGGGGAGGCTTGGTACAGCCG |
| | | | GGGGGGTCCCTGAGACTCTCCTGTGCAGCCTCTGGATTCCCCT |
| | | | TTAGCAGCTATGTCATGAGCTGGGTCCGCCAGGCTCCAGGGAA |
| | | | GGGGCTGGAGTGGGTCTCAGTTATAAGTGTTAGTGGTGGTAGT |
| | | | ACATTCTACGCAGACTCCGTGAGGGGCCGGTTCACCATCTCCA |
| | | | GAGACAATTCCAAGAACACGCTGTATCTGCAAGTGTACAGCCT |
| | | | GCGAGCCGAGGACACGGCCGTATATTACTGTGCGAAAGATGG |
| | | | TTTGGCAGTGGCTGGTTTTGACCACTGGGGCCAGGGAACCCTG |
| | | | GTCACCGTCTCCTCA |
| 296 | ATX-P-574 | VH - nucleotide | GAGGTGCAGCTGGTGGAGTCTGGGGGAGGCTTGGTACAGCGG |
| | | | GGGGGGTCCCTGAGACTCTCCTGTGTAGCCTCTGGATTCACCT |
| | | | TTAGTAGCTATGCCATGAGCTGGGTCCGCCAGGCTCCAGGGAA |
| | | | GGGGCTGGAGTGGGTCTCAGTTATTAGTGGTAGTGGTGGTACC |
| | | | ACATACTACGCAGACTCCGTGAAGGGCCGGTTCACCATCTCCA |
| | | | GAGACGATTCCAAGAAGACACTGTATCTGCAAATGAACAGCC |
| | | | TGAGAGCCGAGGACACGGCCGTATATTATTGTGCGAAAAGGG |
| | | | GTGACTACGGGGACTACATGGACGTCTGGGGCAAAGGGACCA |
| | | | CGGTCACCGTCTCCTCA |
| 297 | ATX-P-565 | VH - nucleotide | CAGGTGCAGCTGGTGGAGTCTGGGGGAGGCGTGGTCCAGCCT |
| | | | GGGAGGTCCCTGAGACTCTCCTGTGCAGCGTCTGGATTCACCT |
| | | | TCAATACCTATGGCATCCACTGGGTCCGCCAGGCTCCAGGCAA |
| | | | GGGACTGGAGTGGGTGGCAGTTATATGGCATGATGGAAGTAG |
| | | | TAAAAACTATGTAGACTCCGTGACGGGCCGATTCACCATCTCC |
| | | | AGAGACAATTCCAAGAACACGGTGTATCTGCAAATGAACAGC |
| | | | CTGAGAGCCGAGGACACGGCTGTTTATTACTGTGCGAGAAGG |
| | | | GGTAACTGGAACTACGGCTTCTACTACTACTACATGGACGTCT |
| | | | GGGGCAAAGGAACCACGGTCACCGTCTCCTCA |
| 298 | ATX-P-578 | VH - nucleotide | GAGGTGCAGCTGGTGGAGTCTGGGGGAGGTTTGGTACAGCCT |
| | | | GGGGGGTCCCTGAGACTCTCCTGTGCAGCCTCTGGATTCACCT |
| | | | TCAGTAGATATAGCATGAACTGGGTCCGCCAGGCTCCAGGGA |
| | | | AGGGGCTGGAGTGGGTTTCATACATTAGTAGTAGTAGTAGTAC |
| | | | CATTTTCCATGCAGACTCTGTGAAGGGCCGATTCACCATCTCC |
| | | | AGAGACAATGCCAAGAACTCACTGTACCTGCAAATGAACAGC |
| | | | CTGAGAGACGAGGACACGGCTGTGTATTACTGTGCGAGAGGG |
| | | | AACTGGAACTACTACTACATGGACGTCTGGGGCAAAGGGACC |
| | | | ACGGTCACCGTCTCCTCA |
| 299 | ATX-P-569 | VH - nucleotide | GAAGTGCAGCTGGTGGAGTCTGGGGGAGGCTTGGCACAGTTT |
| | | | GGCAGGTCCCTGAGACTCTCCTGTGCAGCCTCTGGATTCACCT |
| | | | TTGATGATTATGCCATGCACTGGCTCCGGCAAGCTCCAGGGAT |
| | | | GGGCCTGGAGTGGGTCTCAGGTATTAGTTGGAATAGCGGGAG |
| | | | CATAGGCTATGQGGACTCTGTGAAGGGCCGATTCACCATCGCC |
| | | | AGAGACAACGCCAAGAACTCCGTGCATCTGCAAATGAACAGT |
| | | | CTGAGAGCTGAGGACACGGCCTTGTATTACTGTGCAAAAGATA |
| | | | TAGGAAATGGATATACGAAGGGTGATGTTTTTGATATCTGGGG |
| | | | ACAAGGGACAATGGTCACCGTCTCTTCA |
| 300 | ATX-P-576 | VH - nucleotide | GAAGTGCAGCTGGTGGAGTCTGGGGGAGGCTTGGTACAGCCT |
| | | | GGCAGGTCCCTGAGACTCTCCTGTACAGCCTCTGGATTCACAT |
| | | | TTGATGATTATGAAATGCACTGGGTCCGGCAAGCTCCAGGGAA |
| | | | GGGCCTGGAGTGGGTCTCAGATATTAGTCGGAACAGTGGTAG |
| | | | AATAGGCTATGCGGACTCTGTGAAGGGCCGATTCACCATTTCC |
| | | | AGAGACAACGCCAAGAACTCCCTGTATCTACAAATGAACAGT |
| | | | CTGAGAGCTGAGGACACGGCCTTGTATTACTGTGCAAAAGATA |
| | | | TTTGGGGATCGGGGTACTTTGACTATTGGGGCCAGGGAATCCT |
| | | | GGTCACCGTCTCCTCA |
| 301 | ATX-P-588 | VH - nucleotide | GAAGTGCAGCTGGTGGAGTCCGGGGGAGGCTTGGTACAACCT |
| | | | GGCAGGTCCCTGAGACTCTCCTGTGCAGCCTCTGGATTCACCT |
| | | | TTGAAGATTATGCCATGCACTGGGTCCGGCAAGCTCCAGGGAA |
| | | | GGGCCTGGAGTGGGTCTCAGGTATTAGTTGGAATAGTGGTAGC |
| | | | ATAGGCTATGCGGACTCTGTGAAGGGCCGATTCACCATCTCCA |
| | | | GAGACAACGCCAAGAAATCCCTGTATCTGCAAATGAACAGTC |
| | | | TGAGAGCTGAGGACACGGCCTTATATTACTGTACAAAAGATA |
| | | | GAGATGATGGCTACAACTATGCGGGGGTCTTTGACTACTGGGG |
| | | | CCAGGGAACCCTGGTCACTGTCTCCTCA |
| 302 | ATX-P-596 | VH - nucleotide | GAAGTGCAGCTGGTGGAGTCTGGGGGAGGCTTGGTACAGCCT |
| | | | GGCAGGTCCCTGAGACTCTCCTGTGCAGCCTCTGGGTTCAGTT |
| | | | TTGATGATTATGCCATGCACTGGGTCCGGCAAGTTCCAGGGAA |
| | | | GGGCCTGGAATGGGTCTCAGGTATTAGTTGGACTGGTGGTAGC |
| | | | GTAGGCTATGCGGACCCCGTGAAGGGCCGATTCACCATCTCCA |
| | | | GAGACAACGCCAAGAACTCCCTGTATCTGCAAATGAACAGTCT |
| | | | GAGACCTGAGGACACGGCCTTGTATTACTGTGCAAAAGATATG |
| | | | GGAGACGGGTATAGCAGAAACTGGCCCTTTGACTACTGGGGC |
| | | | CAGGGAACCCTGGTCACCGTCTCCTCA |
| 303 | ATX-P-600 | VH - nucleotide | GAAGTGCAGCTGGTGGAGTCTGGGGGAGGCTTGGTACAGCCT |
| | | | GGCAGGTCCCTGAGACTCTCCTGTGCAGCCTCTGGATTCACCT |
| | | | TTGATGATTATGCCATGCACTGGGTCCGGCAAGTTCCAGGGAG |
| | | | GGGCCTGGAGTGGGTCTCAGGTATTAGTTGGAGTGGTGGCAGC |
| | | | ATAGGCTATGCGGACTCTGTGAAGGGCCGATTCACCATCTCCA |
| | | | GAGACAACTCCAAGAACTCCCTGTATCTGCAAATGAACAGTCT |
| | | | GAGAGCTGAGGACACGGCCTTGTATTACTGTACAAAAGATAG |
| | | | AGAAGATGGATACAACTATGGGGGGGTCTTTGACTACTGGGG |
| | | | CCAGGGAACCCTGGTCACCGTCTCCTCA |
| 304 | ATX-P-564 | VL | DIVMTQSPLSLPVTPGEPASISCRSSQSLLHSTGYNFLDWYLQKPG |
| | | | LSPQLLIYLASNRASGVPDRFSGSGSGTDFTLKISRVEAEDVGVYY |
| | | | CMQALQTPYTFGQGTKLEIK |
| 305 | ATX-P-568 | VL | DIQMTQSPSTLSASVGDRVTITCRASQSISTWLAWYQQKPGKAPK |
| | | | LLIHKASGLESGVPSRFSGSGSGTEFTLTISSLQPDDFATYYCQQY |
| | | | NSFSLIFGGGTRVEIK |
| 306 | ATX-P-574 | VL | DIQMTQSPSSVSASVGDRVTITCRASQGITTWLAWYQQKPGKAP |
| | | | KLLIFGASSLESGVPSRFSGSGSGTDFTLTISSLQPEDFATYYCQQA |
| | | | NSFPWTFGQGTKVEIK |
| 307 | ATX-P-565 | VL | DIVMTQSPLSLPVTPGEPASISCRSSQRLLHSSGYNYLDWYLQKPG |
| | | | QSPQLLIYLGSSRASGVPDRFSGSGSGTDFTLKISRVEAEDVGVYY |
| | | | CMQGLQTPPTFGQGTKVEIK |
| 308 | ATX-P-578 | VL | DIQMTQSPSTLSASVGDRVTISCRASQSVNSWLAWYQQKPGKAP |
| | | | KLLIYKASNLEGGVPSSFSGSGSGTEFTLTISSLQPDDFATYYCQQ |
| | | | YDTSWTFGQGTKVEIK |
| 309 | ATX-P-569 | VL | DIQMTQSPSTLSASVGDSVTITCRASQNIITWLAWYQQKPGKAPN |
| | | | LLIYKASSLESGVPSRFSGSGSGTEFTLTISSLQPDDFATYYCQQYS |
| | | | SYSYTFGQGTKLEIK |
| 310 | ATX-P-576 | VL | DIQMTQSPSSVSASVGDRVTITCRTSQGIRNWLAWYQQKPGKAP |
| | | | KLLIYAASSLQTGGPSRFSGSGSGTDFTLTISSLQPEDFATYYCQQ |
| | | | ANSFPYTFGQGTKLEIK |
| 311 | ATX-P-588 | VL | EIVLTQSPATLSVSPGERATLSCRASQSVSSNLAWYQQKPGQAPR |
| | | | LLIYGASSRATGIPDRFSGSGSGTDFTLTISRLEPEDFAVYYCQQY |
| | | | GSSFTFGPGTKVDIK |
| 312 | ATX-P-596 | VL | DIVMTQSPDSLAVSLGERATINCKSSQSVLYSSNNKNYLAWYQQ |
| | | | KPGQPPKLLIYWASTRESGVPDRFSGSGSGTDFTLTISSLQAEDVA |
| | | | VYYCQQYGSSPMYTFGQGTKLEIK |
| 313 | ATX-P-600 | VL | EIVLTQSPGTLSLSPGERATLSCRASQNVRGSYLAWYQQKPGQAP |
| | | | RLLIYGASNRATDIPDRFSGSGSGTDFTLTISGLEPEDFVVYYCQQ |
| | | | YGSSYTFGQGTKVEIK |
| 314 | ATX-P-564 | VL - nucleotide | GATATTGTGATGACTCAGTCTCCACTCTCCCTGCCCGTCACCCC |
| | | | TGGAGAGCCGGCCTCCATCTCCTGCAGGTCTAGTCAGAGCCTC |
| | | | CTGCATAGTACTGGATACAACTTTTTGGATTGGTACCTGCAGA |
| | | | AGCCAGGGCTGTCTCCACAGCTCCTGATCTATTTGGCTTCTAAT |
| | | | CGGGCCTCCGGGGTCCCTGACAGGTTCAGTGGCAGTGGATCAG |
| | | | GCACAGATTTTACACTGAAAATCAGCAGAGTGGAGGCTGAGG |
| | | | ATGTTGGGGTTTATTACTGCATGCAAGCTCTACAAACTCCGTA |
| | | | CACTTTTGGCCAGGGGACCAAGCTGGAGATCAAA |
| 315 | ATX-P-568 | VL - nucleotide | GACATCCAGATGACCCAGTCTCCTTCCACCCTGTCTGCCTCTGT |
| | | | AGGAGACAGAGTCACCATCACTTGCCGGGCCAGCCAGAGTAT |
| | | | TAGTACCTGGTTGGCCTGGTATCAGCAGAAACCAGGGAAAGC |
| | | | CCCTAAGCTCCTGATCCATAAGGCGTCTGGTTTAGAAAGTGGG |
| | | | GTCCCATCAAGATTCAGCGGCAGTGGATCTGGGACAGAATTCA |
| | | | CTCTCACCATCAGCAGCCTGCAGCCTGATGATTTTGCAACTTA |
| | | | TTACTGCCAACAGTATAATAGTTTTTCTCTCATTTTCGGCGGAG |
| | | | GGACCAGGGTGGAGATCAAA |
| 316 | ATX-P-574 | VL - nucleotide | GACATCCAGATGACCCAGTCTCCATCTTCCGTGTCTGCATCTGT |
| | | | AGGAGACAGAGTCACCATCACTTGTCGGGCGAGTCAGGGTAT |
| | | | TACCACCTGGTTAGCCTGGTATCAGCAGAAACCAGGGAAAGC |
| | | | CCCTAAGCTCCTGATCTTTGGTGCATCTAGTTTGGAAAGTGGG |
| | | | GTCCCATCAAGGTTCAGCGGCAGTGGATCTGGGACAGATTTCA |
| | | | CTCTCACCATCAGCAGCCTGCAGCCTGAAGATTTTGCAACTTA |
| | | | CTATTGTCAACAGGCTAACAGTTTCCCGTGGACGTTCGGCCAA |
| | | | GGGACCAAGGTGGAAATCAAA |
| 317 | ATX-P-565 | VL - nucleotide | GATATTGTGATGACTCAGTCTCCACTCTCCCTGCCCGTCACCCC |
| | | | TGGAGAGCCGGCCTCCATCTCCTGCAGGTCTAGTCAGCGCCTC |
| | | | CTGCATAGTAGTGGATACAATTATTTGGATTGGTATCTGCAGA |
| | | | AGCCAGGACAGTCTCCACAGCTCCTGATCTATTTGGGTTCTAG |
| | | | TCGGGCCTCCGGGGTCCCTGACAGGTTCAGTGGCAGTGGATCA |
| | | | GGCACAGATTTTACACTGAAAATCAGCAGGGTGGAGGCTGAG |
| | | | GATGTTGGGGTTTATTACTGCATGCAAGGTCTACAAACTQCTC |
| | | | CGACGTTCGGCCAAGGGACCAAGGTGGAAATCAAA |
| 318 | ATX-P-578 | VL - nucleotide | GACATCCAGATGACCCAGTCTCCTTCCACCCTGTCTGCATCTGT |
| | | | AGGAGACAGAGTCACCATCAGTTGCCGGGCCAGTCAGAGTGT |
| | | | TAATAGCTGGTTGGCCTGGTATCAGCAGAAACCAGGGAAAGC |
| | | | CCCTAAACTCCTGATCTATAAGGCGTCTAATTTAGAAGGTGGG |
| | | | GTCCCATCAAGTTTCAGCGGCAGTGGATCTGGGACAGAATTCA |
| | | | CTCTCACCATCAGCAGCCTGCAGCCTGATGATTTTGCAACTTA |
| | | | TTACTGCCAACAATATGATACTTCTTGGACGTTCGGCCAAGGG |
| | | | ACCAAGGTGGAGATCAAA |
| 319 | ATX-P-569 | VL - nucleotide | GACATCCAGATGACCCAGTCTCCTTCCACCCTGTCTGCATCTGT |
| | | | AGGAGACAGCGTCACCATCACTTGCCGGGCCAGTCAGAATATT |
| | | | ATTACCTGGTTGGCTTGGTATCAGCAGAAACCAGGGAAAGCCC |
| | | | CTAACCTCCTGATCTATAAGGCGTCTAGTTTAGAAAGTGGGGT |
| | | | CCCATCAAGGTTCAGCGGCAGTGGATCTGGGACAGAATTCACT |
| | | | CTCACCATCAGCAGCCTGCAGCCTGATGATTTTGCAACTTATT |
| | | | ACTGCCAACAGTATAGTAGTTATTCGTACACTTTTGGCCAGGG |
| | | | GACCAAGCTGGAGATCAAA |
| 320 | ATX-P-576 | VL - nucleotide | GACATCCAGATGACCCAGTCTCCATCTTCCGTGTCTGCATCTGT |
| | | | AGGAGACAGAGTCACCATCACTTGTCGGACGAGTCAGGGTAT |
| | | | TAGAAACTGGTTAGCCTGGTATCAGCAGAAACCAGGGAAAGC |
| | | | CCCTAAACTCCTGATCTATGCTGCATCCAGTTTGCAAACTGGG |
| | | | GGCCCATCAAGGTTCAGCGGCAGTGGATCTGGGACAGATTTCA |
| | | | CTCTCACCATCAGCAGCCTGCAGCCTGAAGATTTTGCAACTTA |
| | | | CTATTGTCAACAGGCTAACAGTTTCCCGTACACTTTTGGCCAG |
| | | | GGGACCAAGCTGGAGATCAAA |
| 321 | ATX-P-588 | VL - nucleotide | GAAATTGTGTTGACGCAGTCTCCAGCCACCCTGTCTGTGTCTC |
| | | | CAGGGGAAAGAGCCACCCTCTCCTGCAGGGCCAGTCAGAGTG |
| | | | TTAGCAGCAACTTAGCCTGGTACCAGCAGAAACCTGGCCAGG |
| | | | CTCCCAGGCTCCTCATCTATGGTGCATCCAGCAGGGCCACTGG |
| | | | CATCCCAGACAGGTTCAGTGGCAGTGGGTCTGGGACAGACTTC |
| | | | ACTCTCACCATCAGCAGACTGGAGCCTGAAGATTTTGCAGTGT |
| | | | ATTACTGTCAGCAGTATGGTAGCTCATTCACTTTCGGCCCTGG |
| | | | GACCAAAGTGGATATCAAA |
| 322 | ATX-P-596 | VL - nucleotide | GACATCGTGATGACTCAGTCTCCAGACTCCCTGGCTGTGTCTC |
| | | | TGGGCGAGAGGGCCACCATCAACTGCAAGTCCAGCCAGAGTG |
| | | | TTTTATACAGCTCCAACAATAAGAACTACTTAGCTTGGTACCA |
| | | | GCAGAAACCAGGACAGCCTCCTAAGCTGCTCATTTACTGGGCA |
| | | | TCTACCCGGGAATCCGGGGTCCCTGACCGATTCAGTGGCAGCG |
| | | | GGTCTGGGACAGATTTCACTCTCACCATCAGCAGCCTGCAGGC |
| | | | TGAAGATGTGGCAGTTTATTACTGTCAGCAGTATGGTAGCTCA |
| | | | CCCATGTACACTTTTGGCCAGGGGACCAAGCTGGAGATCAAA |
| 323 | ATX-P-600 | VL - nucleotide | GAAATTGTGTTGACGCAGTCTCCAGGCACCCTGTCTTTGTCTCC |
| | | | AGGGGAAAGAGCCACCCTCTCCTGCAGGGCCAGTCAGAATGT |
| | | | TAGGGGCAGCTACTTAGCCTGGTACCAGCAGAAACCTGGCCA |
| | | | GGCTCCCAGGCTCCTCATATATGGTGCATCCAACAGGGCCACT |
| | | | GACATCCCAGACAGGTTCAGTGGCAGTGGGTCTGGGACAGAC |
| | | | TTCACTCTCACCATCAGCGGCCTGGAGCCTGAAGATTTTGTAG |
| | | | TGTATTACTGTCAGCAGTATGGTAGTTCGTACACTTTTGGCCA |
| | | | GGGGACCAAGGTGGAGATCAAA |
| 324 | ATX-P-571 | VH | QVQLQESGPGLVKPSGTLSLTCAVSGGSISSRNWWSWVRQPPGK |
| | | | GLEWIGEIYHGGTTNYNPSLKSRVTISVDKSKNQFSLKLSSVTAA |
| | | | DTAVYYCARGGLRLGELSPNAFDIWGQGTMVTVSS |
| 325 | ATX-P-577 | VH | QVQLQESGPGLVKPSGTLSLTCAVSGGSISSSNWWTWVRQPPGK |
| | | | GLEWIGETYHDGSTNYNPSLKSRVTISVDKSKNQFSLKLSSVTAA |
| | | | DTAVYYCLEFYFDYWGQGTLVTVSS |
| 326 | ATX-P-579 | VH | QVQLQESGPGLVKPSGTLSLTCAVSGASISTSNWWTWVRQPPGK |
| | | | GLEWIGEIYHDGSTNYSPSLQSRVTISVDKSKNQFSLTLTSVTAAD |
| | | | TAIYYCGDFYIDVWGNGTTVTVSS |
| 327 | ATX-P-580 | VH | QVQLQESGPGLVKPSGTLSLTCAVSGGSISTSDWWTWVRQPPGK |
| | | | GLEWIGEIYHSGSTNYNPSLKSRVTISVDKSKNQFSLKLSSVTAAD |
| | | | TAVYYCADWYFDLWGRGTLVTVSS |
| 328 | ATX-P-582 | VH | QVQLQESGPGLVKPSGTLSLTCAVSGGSISISKWWSWVRQPPGKG |
| | | | LEWIGEIYHTGSTNYHPSLKSRVTISVDKSKNQFYLKLNSVTAAD |
| | | | TAVYYCARDELRLGELSPSLDIWGQGTMVTVSS |
| 329 | ATX-P-598 | VH | QVQLQESGPGLVKPSGTLSLTCAVSGGSISSSNWWSWVRQSPGK |
| | | | GLEWIGEIYHSGTTNYNPSLKSRVTISVDKSKNQFSLKLSSATAAD |
| | | | TAVYYCARDRLRLGESSSDVFDIWGQGTMVTVSS |
| 330 | ATX-P-603 | VH | QVQLQESGPGLVKPSGTLSLTCAVSGGSISGSHWWSWFRQPPGK |
| | | | GLEWIGEIYHTGSTNKNPSLKSRVTISLDKSKNQFSLRLRSVIDAD |
| | | | TAVYYCARDQYTGSPRDAFDIWGQGTMVTVSS |
| 331 | ATX-P-608 | VH | QVQLQESGPGLVKPSGTLSLTCAVSGGSISSSNWWSWVRQSPGK |
| | | | GLEWIGEIYHTGSTNKKPSLKSRVIISVDKSKNQFSLKLISVNAAD |
| | | | TAIYYCARDREGGTTEAFDIWGQGTMVTVSS |
| 332 | ATX-P-609 | VH | QVQLQESGPGLVKPSGTLSLTCAVSGGSISSSNWWSWVRQPPGK |
| | | | GLEWIGEIYHSGSTNYNPSLKSRVTISVDKSKNQFSLKLRSVTAAD |
| | | | TAVYYCARDQLSSGPTDAFDIWGQGTMVTVSS |
| 333 | ATX-P-563 | VH | QVQLQESGPGLMKPSETLSLTCTVSGGSISSHYWSWIRQPPGKGL |
| | | | EWIGYIFYSGNINYNPSLKSRVTISVDTSKNQFSLKLRSVTTADTA |
| | | | VYYCARDRGGSYSYHFDCWGQGTLVTVSS |
| 334 | ATX-P-570 | VH | QVQLQESGPGLVKPSETMSLTCTVSGGSFTGYYWSWIRQPPGKG |
| | | | LEWIGYVHYSGGTKSNPSLKSRVTISVDTSKNQFSLKLSSVTAAD |
| | | | TAVYYCARERYEWNSYFDYWGQGTLVTVSS |
| 335 | ATX-P-571 | VH - nucleotide | CAGGTGCAGCTGCAGGAGTCGGGCCCAGGACTGGTGAAGCCT |
| | | | TCGGGGACCCTGTCCCTCACCTGTGCTGTCTCGGGTGGCTCCA |
| | | | TCAGTAGTCGTAACTGGTGGAGTTGGGTCCGCCAGCCCCCAGG |
| | | | GAAGGGGCTGGAGTGGATTGGGGAAATCTATCATGGTGGGAC |
| | | | CACCAACTACAACCCGTCCCTTAAGAGTCGAGTCACCATATCA |
| | | | GTAGACAAGTCCAAGAACCAATTCTCCCTGAAGCTGAGTTCTG |
| | | | TGACCGCCGCGGACACGGCCGTGTATTACTGTGCGAGAGGGG |
| | | | GATTACGTTTGGGGGAGTTATCGCCTAATGCTTTTGATATCTG |
| | | | GGGACAAGGGACAATGGTCACCGTCTCTTCA |
| 336 | ATX-P-577 | VH - nucleotide | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCAGTAGTAACTGGTGGACTTGGGTCCGCCAGCCCCCAGG |
| | | | GAAGGGGCTGGAGTGGATTGGGGAAACCTATCATGATGGGAG |
| | | | CACCAACTACAACCCGTCCCTCAAGAGTCGAGTCACCATTTCA |
| | | | GTAGACAAGTCCAAGAACCAATTCTCCCTGAAACTGAGTTCTG |
| | | | TGACCGCCGCGGACACGGCCGTGTATTACTGTTTGGAATTCTA |
| | | | CTTTGACTACTGGGGCCAGGGAACCCTGGTCACCGTCTCCTCA |
| 337 | ATX-P-579 | VH - nucleotide | CAGGTGCAGCTGCAGGAGTCGGGCCCAGGACTGGTGAAGCCT |
| | | | TCGGGGACCCTGTCCCTCACCTGCGCTGTGTCTGGTGCCTCCAT |
| | | | CAGCACAAGTAATTGGTGGACTTGGGTCCGCCAGCCCCCAGG |
| | | | GAAGGGGCTGGAGTGGATTGGTGAAATCTATCATGATGGGAG |
| | | | CACCAACTACAGCCCGTCCCTCCAGAGTCGAGTCACCATATCA |
| | | | GTAGACAAGTCCAAGAACCAGTTCTCCCTGACGCTGACCTCTG |
| | | | TGACCGCCGCGGACACGGCCATTTATTATTGTGGTGACTTCTA |
| | | | CATAGACGTCTGGGGCAACGGGACCACGGTCACCGTCTCCTCA |
| 338 | ATX-P-580 | VH - nucleotide | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCACTAGTGACTGGTGGACTTGGGTCCGCCAGCCCCCAGGG |
| | | | AAGGGGCTGGAGTGGATTGGGGAAATCTATCATAGTGGGAGC |
| | | | ACCAACTACAACCCGTCCCTCAAGAGTCGAGTCACCATATCAG |
| | | | TAGACAAATCCAAGAACCAGTTCTCCCTGAAGCTGAGCTCTGT |
| | | | GACCGCCGCGGACACGGCCGTGTATTACTGTGCGGACTGGTAC |
| | | | TTCGATCTCTGGGGCCGTGGCACCCTGGTCACTGTCTCCTCA |
| 339 | ATX-P-582 | VH - nucleotide | CAGGTGCAGCTGCAGGAGTCGGGCCCAGGACTGGTGAAGCCT |
| | | | TCGGGGACCCTGTCCCTCACCTGCGCTGTGTCTGGTGGCTCCA |
| | | | TCAGCATTAGTAAATGGTGGAGTTGGGTCCGCCAGCCCCCAGG |
| | | | GAAGGGACTGGAGTGGATTGGGGAAATCTATCATACTGGGAG |
| | | | CACCAACTACCACCCGTCCCTCAAGAGCCGAGTCACCATATCA |
| | | | GTAGACAAGTCCAAGAACCAATTCTACCTGAAGCTGAACTCTG |
| | | | TGACCGCCGCGGACACGGCCGTATATTACTGTGCGAGAGATG |
| | | | AATTACGGTTGGGGGAGTTATCGCCCTCTCTTGATATCTGGGG |
| | | | ACAAGGGACAATGGTCACCGTCTCTTCA |
| 340 | ATX-P-598 | VH - nucleotide | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCAGTAGTAACTGGTGGAGTTGGGTCCGCCAGTCCCCAGG |
| | | | GAAGGGGCTGGAATGGATTGGGGAAATCTATCATAGTGGGAC |
| | | | CACCAACTACAACCCGTCCCTCAAGAGTCGAGTCACCATATCA |
| | | | GTAGACAAGTCCAAGAACCAGTTCTCCCTGAAGCTGAGCTCTG |
| | | | CGACCGCCGCGGACACGGCCGTGTATTATTGTGCGAGAGATA |
| | | | GATTACGTTTGGGGGAGTCATCGTCCGATGTTTTTGATATCTG |
| | | | GGGACAAGGGACAATGGTCACCGTCTCTTCA |
| 341 | ATX-P-603 | VH - nucleotide | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCGGTAGTCACTGGTGGAGTTGGTTCCGCCAGCCCCCAGGA |
| | | | AAGGGGCTGGAGTGGATTGGGGAAATCTATCATACTGGGAGC |
| | | | ACCAACAAAAACCCGTCCCTCAAGAGTCGAGTCACTATCTCAT |
| | | | TAGACAAGTCCAAGAATCAGTTCTCCCTGAGGCTGAGGTCTGT |
| | | | GACCGACGCGGACACGGCCGTGTATTACTGTGCGAGAGATCA |
| | | | GTATACTGGGAGCCCAAGGGATGCTTTTGATATCTGGGGACAA |
| | | | GGGACAATGGTCACCGTCTCCTCA |
| 342 | ATX-P-608 | VH - nucleotide | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCAGTAGTAACTGGTGGAGTTGGGTCCGCCAGTCCCCAGG |
| | | | GAAGGGGCTGGAGTGGATTGGGGAAATCTATCATACTGGGAG |
| | | | CACCAACAAAAAACCGTCCCTCAAGAGTCGAGTCATCATATCA |
| | | | GTAGACAAGTCCAAGAACCAGTTCTCCCTGAAACTGATCTCTG |
| | | | TGAACGCCGCGGACACGGCCATATATTACTGTGCGAGAGATC |
| | | | GGGAAGGGGGAACTACTGAAGCTTTTGATATCTGGGGACAAG |
| | | | GGACAATGGTCACCGTCTCTTCA |
| 343 | ATX-P-609 | VH - nucleotide | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCAGTAGTAACTGGTGGAGTTGGGTCCGCCAGCCCCCAGG |
| | | | GAAGGGGCTGGAGTGGATTGGGGAAATCTATCATAGTGGGAG |
| | | | CACCAACTACAACCCGTCCCTCAAGAGTCGAGTCACCATATCA |
| | | | GTAGACAAGTCCAAGAACCAGTTCTCCCTGAAGCTGAGGTCTG |
| | | | TGACCGCCGCGGACACGGCCGTTTATTACTGTGCGAGAGATCA |
| | | | ACTAAGTAGCGGACCTACGGATGCTTTTGATATCTGGGGACAA |
| | | | GGGACAATGGTCACCGTCTCTTCA |
| 344 | ATX-P-563 | VH - nucleotide | CAGGTGCAGCTGCAGGAGTCGGGCCCAGGACTGATGAAGCCT |
| | | | TCGGAGACCCTGTCCCTCACCTGCACTGTCTCTGGTGGCTCCAT |
| | | | CAGTAGTCACTACTGGAGCTGGATCCGGCAACCCCCAGGGAA |
| | | | GGGACTGGAGTGGATTGGATATATCTTTTACAGTGGGAACATA |
| | | | AATTACAACCCCTCCCTCAAGAGTCGAGTCACCATATCAGTAG |
| | | | ACACGTCCAAGAACCAGTTCTCCCTGAAGCTGAGGTCTGTGAC |
| | | | CACTGCGGACACGGCCGTATATTACTGTGCGAGAGATCGAGGT |
| | | | GGGAGCTATTCGTACCACTTTGACTGCTGGGGCCAGGGAACCC |
| | | | TGGTCACCGTCTCCTCA |
| 345 | ATX-P-570 | VH - nucleotide | CAGGTGCAGCTGCAGGAGTCGGGCCCAGGACTGGTGAAGCCT |
| | | | TCGGAGACTATGTCCCTCACCTGCACTGTCTCTGGTGGCTCCTT |
| | | | CACTGGATACTACTGGAGTTGGATCCGGCAGCCCCCAGGGAA |
| | | | GGGACTGGAGTGGATTGGATATGTCCATTACAGTGGGGGCAC |
| | | | CAAAAGCAACCCCTCCCTCAAGAGTCGAGTCACCATATCAGTA |
| | | | GACACGTCCAAGAACCAGTTTTCCCTGAAGCTGAGCTCTGTGA |
| | | | CCGCTGCGGACACGGCCGTGTATTACTGTGCGAGAGAGAGGT |
| | | | ATGAGTGGAACTCCTACTTTGACTACTGGGGCCAGGGAACCCT |
| | | | GGTCACCGTCTCCTCA |
| 346 | ATX-P-571 | VL | DIQMTQSPSSLSASVGDRVTITCRASQSISSYLNWYQQKPGKAPK |
| | | | VLIYAASSLQSGIPSRFSGSGSGTDFTLTISSLQPEDFATYYCQETY |
| | | | SVPLTFGGGTKVEIK |
| 347 | ATX-P-577 | VL | DIQMTQSPSSLSASVGDRVTITCRASQDIRNDLGWYQQKPGKVPK |
| | | | RLIYAASSLQSGVPSRFSGSGSGTEFTLTISSLQPEDFASYYCLQYN |
| | | | NYPFTLGPGTKVDIK |
| 348 | ATX-P-579 | VL | DIQMTQSPSSLSASVGDRVTITCRASQGIRKDLGWYQQKPGKAPK |
| | | | RLIYTASTLQSGVPSRFSGSGSGTEFTLTISSLQPEDFATYYCLQYN |
| | | | NYPFTFGPGTKVDIK |
| 349 | ATX-P-580 | VL | DIQMTQSPSSLSASVGDRVTITCRASQGIRNDLGWYQQKPGKAPK |
| | | | RLIYAASSLQSGVPSRFSGSGSGTEFTLAISSLQPEDFATYYCLHEN |
| | | | SYPFTFGPGTK VDIK |
| 350 | ATX-P-582 | VL | DIQMTQSPSSLSASVGDRVTITCRASQNINNYLNWFRQKPGKAPK |
| | | | LLIFAASSLQSGVPSRFSGSESGTDFTLTISSLQPEDFATYYCQQSY |
| | | | STPLTFGGGTK VEIK |
| 351 | ATX-P-598 | VL | DIVMTQSPDSLAVSLGERATINCKSSQSVLYSSNNKNYLAWYQQ |
| | | | KPGQPPELLIYWASTRESGVPDRFSGSGSGTDFTLTISSLQAEDMA |
| | | | VYYCQQFYNTPYTFGQGTKVDIK |
| 352 | ATX-P-603 | VL | EIVLTQSPGTLSLSPGERATLSCRASQSVSSSYLAWYQQKPGQAP |
| | | | RLLIYGASSRATGIPDRFSGSGSGTDFTLTISRLEPEDFAVYYCQH |
| | | | YGNSPLTFGGGTKVEIK |
| 353 | ATX-P-608 | VL | EIVMTQSPATLSVSPGERATLSCRASQSVSSNLAWYQQKPGQAPR |
| | | | LLIYGASTRATGIPVRFSGSGSGTQFTLTISSLQSEDFAVYYCQQY |
| | | | DNWRTFGQGTRLEIK |
| 354 | ATX-P-609 | VL | EIVMTQSPATLSVSPGERATLSCRASQSVSSNLAWYQQKPGQAPR |
| | | | LLIYGASTRATGIPVRFSGSGSGTQFTLTISSLQSEDFAVYYCQQY |
| | | | DNWRTFGQGTKLEIK |
| 355 | ATX-P-563 | VL | DIQMTQSPSSVSASVGDRVTITCRASQSFAGWLAWYQQKPGKAP |
| | | | KLLIYAASTLQSGVPSRFSGSGSGTDFTLTISSLQPEDFATYYCQQ |
| | | | ANSFPLTFGGGTKVEIK |
| 356 | ATX-P-570 | VL | DIVMTQSPDSLAVSLGERATINCKSSQSVLYSSNNKNYLAWYHQ |
| | | | KPGQPPKLLIYWASTRESGVPDRFSGSGSGTDFTLTISSLQAEDVA |
| | | | VYYCQQYYSSPRTFGQGTKVEIK |
| 357 | ATX-P-571 | VL - nucleotide | GACATCCAGATGACCCAGTCTCCATCCTCCCTGTCTGCATCTGT |
| | | | GGGAGACAGAGTCACCATCACTTGCCGGGCAAGTCAGAGCAT |
| | | | TAGCAGCTATTTAAATTGGTATCAGCAGAAACCAGGGAAAGC |
| | | | CCCTAAGGTCCTGATCTATGCTGCATCCAGTTTGCAGAGTGGG |
| | | | ATCCCATCACGGTTCAGTGGCAGTGGATCTGGGACAGATTTCA |
| | | | CTCTCACCATCAGCAGTCTGCAACCTGAAGATTTTGCAACTTA |
| | | | CTACTGTCAAGAGACTTACAGTGTTCCGCTCACTTTCGGCGGA |
| | | | GGGACCAAGGTGGAGATCAAA |
| 358 | ATX-P-577 | VL - nucleotide | GACATCCAGATGACCCAGTCTCCATCCTCCCTGTCTGCATCTGT |
| | | | AGGAGACAGAGTCACCATCACTTGCCGGGCAAGTCAGGACAT |
| | | | TAGAAATGATTTAGGCTGGTATCAGCAGAAACCAGGGAAAGT |
| | | | CCCTAAGCGCCTGATCTATGCTGCATCTAGTTTGCAAAGTGGG |
| | | | GTCCCATCAAGGTTCAGCGGCAGTGGATCTGGGACAGAATTCA |
| | | | CTCTCACAATCAGCAGCCTGCAGCCTGAAGATTTCGCAAGTTA |
| | | | TTACTGTCTACAGTATAATAATTATCCATTCACTCTCGGCCCTG |
| | | | GGACCAAAGTGGATATCAAA |
| 359 | ATX-P-579 | VL - nucleotide | GACATCCAGATGACCCAGTCTCCATCCTCCCTGTCTGCATCTGT |
| | | | AGGAGACAGAGTCACCATCACTTGCCGGGCAAGTCAGGGCAT |
| | | | TAGAAAAGATTTAGGCTGGTATCAGCAGAAACCAGGGAAAGC |
| | | | CCCTAAGCGCCTGATCTATACTGCATCCACTTTACAAAGTGGG |
| | | | GTCCCATCAAGGTTCAGCGGCAGTGGATCTGGGACAGAATTCA |
| | | | CTCTCACAATCAGCAGCCTGCAGCCTGAAGATTTTGCAACTTA |
| | | | TTACTGTCTACAGTATAATAATTACCCATTCACTTTCGGCCCTG |
| | | | GGACCAAAGTGGATATCAAA |
| 360 | ATX-P-580 | VL - nucleotide | GACATCCAGATGACCCAGTCTCCATCCTCCCTGTCTGCTTCTGT |
| | | | CGGAGACAGAGTCACCATCACTTGCCGGGCAAGTCAGGGCAT |
| | | | TAGAAATGATTTAGGCTGGTATCAGCAGAAACCAGGGAAAGC |
| | | | CCCTAAGCGCCTGATCTATGCTGCATCCAGTTTGCAAAGTGGG |
| | | | GTCCCATCAAGGTTCAGCGGCAGTGGATCTGGGACAGAATTCA |
| | | | CTCTCGCAATCAGCAGCCTGCAGCCTGAAGATTTTGCAACTTA |
| | | | TTACTGTCTACACTTTAATAGTTACCCATTCACTTTCGGCCCTG |
| | | | GGACCAAAGTGGATATCAAA |
| 361 | ATX-P-582 | VL - nucleotide | GACATCCAGATGACCCAGTCTCCATCCTCCCTGTCTGCATCTGT |
| | | | AGGAGACAGAGTCACCATCACTTGCCGGGCAAGTCAGAACAT |
| | | | TAACAACTATTTAAATTGGTTCCGGCAGAAACCAGGGAAAGC |
| | | | CCCTAAGCTCCTGATCTTTGCTGCATCCAGTTTACAAAGTGGG |
| | | | GTCCCATCAAGATTCAGTGGCAGTGAATCTGGGACAGATTTCA |
| | | | CTCTCACCATCAGCAGTCTGCAACCTGAGGATTTTGCGACTTA |
| | | | CTACTGTCAACAGAGTTACAGTACCCCTCTCACTTTCGGCGGA |
| | | | GGGACCAAGGTGGAGATCAAA |
| 362 | ATX-P-598 | VL - nucleotide | GACATCGTGATGACTCAGTCTCCAGACTCCCTGGCTGTGTCTC |
| | | | TGGGCGAGAGGGCCACCATCAACTGCAAGTCCAGCCAGAGTG |
| | | | TTTTATACAGCTCCAACAATAAGAACTACTTAGCTTGGTACCA |
| | | | GCAGAAACCAGGACAGCCTCCTGAGCTGCTCATTTACTGGGCA |
| | | | TCTACCCGGGAATCCGGGGTCCCTGACCGATTCAGTGGCAGCG |
| | | | GGTCTGGGACAGATTTCACTCTCACCATCAGCAGCCTGCAGGC |
| | | | TGAAGATATGGCAGTTTATTACTGTCAACAATTTTATAATACT |
| | | | CCGTACACTTTTGGCCAGGGGACCAAAGTGGATATCAAA |
| 363 | ATX-P-603 | VL - nucleotide | GAAATTGTGTTGACGCAGTCTCCAGGCACCCTGTCTTTGTCTCC |
| | | | AGGGGAAAGAGCCACCCTCTCCTGCAGGGCCAGTCAGAGTGT |
| | | | TAGCAGCAGCTACTTAGCCTGGTACCAGCAGAAACCTGGCCA |
| | | | GGCTCCCAGGCTCCTCATCTATGGTGCATCCAGCAGGGCCACT |
| | | | GGCATCCCAGACAGGTTCAGTGGCAGTGGGTCTGGGACAGAC |
| | | | TTCACTCTCACCATCAGCAGACTGGAGCCTGAAGATTTTGCAG |
| | | | TGTATTACTGTCAGCATTATGGTAACTCACCGCTCACTTTCGGC |
| | | | GGAGGGACCAAGGTGGAGATCAAA |
| 364 | ATX-P-608 | VL - nucleotide | GAAATAGTGATGACGCAGTCTCCAGCCACCCTGTCTGTGTCTC |
| | | | CAGGGGAAAGAGCCACCCTCTCCTGCAGGGCCAGTCAGAGTG |
| | | | TTAGCAGCAACTTAGCCTGGTACCAGCAGAAACCTGGCCAGG |
| | | | CTCCCAGGCTCCTCATCTATGGTGCATCCACCAGGGCCACTGG |
| | | | TATCCCAGTCAGGTTCAGTGGCAGTGGGTCTGGGACACAGTTC |
| | | | ACTCTCACCATCAGCAGCCTGCAGTCTGAAGATTTTGCAGTTT |
| | | | ATTATTGTCAGCAATATGATAACTGGCGGACGTTCGGCCAAGG |
| | | | GACACGACTGGAGATTAAA |
| 365 | ATX-P-609 | VL - nucleotide | GAAATAGTGATGACGCAGTCTCCAGCCACCCTGTCTGTGTCTC |
| | | | CAGGGGAAAGAGCCACCCTCTCCTGCAGGGCCAGTCAGAGTG |
| | | | TTAGCAGCAACTTAGCCTGGTACCAGCAGAAACCTGGCCAGG |
| | | | CTCCCAGGCTCCTCATCTATGGTGCATCCACCAGGGCCACTGG |
| | | | TATCCCAGTCAGGTTCAGTGGCAGTGGGTCTGGGACACAGTTC |
| | | | ACTCTCACCATCAGCAGCCTGCAGTCTGAAGATTTTGCAGTTT |
| | | | ATTATTGTCAGCAATATGATAACTGGCGGACGTTCGGCCAAGG |
| | | | GACCAAGCTGGAAATCAAA |
| 366 | ATX-P-563 | VL - nucleotide | GACATCCAGATGACCCAGTCTCCATCTTCCGTGTCTGCATCTGT |
| | | | CGGAGACAGAGTCACCATCACTTGTCGGGCGAGTCAGAGTTTT |
| | | | GCCGGCTGGTTAGCCTGGTATCAGCAGAAACCAGGGAAAGCC |
| | | | CCTAAGCTCCTGATATATGCTGCATCCACTTTGCAAAGTGGGG |
| | | | TCCCATCAAGGTTCAGCGGCAGTGGATCTGGGACAGATTTCAC |
| | | | TCTCACCATCAGCAGCCTGCAGCCTGAAGATTTTGCAACTTAC |
| | | | TATTGTCAACAGGCTAACAGTTTCCCTCTCACTTTCGGCGGAG |
| | | | GGACCAAGGTGGAGATCAAA |
| 367 | ATX-P-570 | VL - nucleotide | GACATCGTGATGACCCAGTCTCCAGACTCCCTGGCTGTGTCTC |
| | | | TGGGCGAGAGGGCCACCATCAACTGCAAGTCCAGTCAGAGTG |
| | | | TTTTATACAGCTCCAACAATAAGAATTACTTAGCTTGGTACCA |
| | | | CCAGAAACCAGGACAGCCTCCTAAGCTGCTCATTTACTGGGCA |
| | | | TCTACCCGGGAATCCGGGGTCCCTGACCGATTCAGTGGCAGCG |
| | | | GGTCTGGGACAGATTTCACTCTCACCATCAGCAGCCTGCAGGC |
| | | | TGAAGATGTGGCAGTTTATTACTGTCAGCAATACTATAGTAGT |
| | | | CCTCGGACGTTCGGCCAAGGGACCAAGGTGGAAATCAAA |
| 368 | ATX-P-560 | VH | QVQLQQSGPGLVKPSQTLSLTCAISGDSVSSNTAAWNWIRQSPSR |
| | | | GLEWLGRTYYRSKWYNDYAVSVKSRITINPDTSKNQFSLQLSSVT |
| | | | PQDTAIYYCASDNWNNGGPGFDPWGQGTLVTVSS |
| 369 | ATX-P-561 | VH | QVQLQQSGPGLVKPSQTLSLTCAISGDSVSSNSAAWNWIRQSPSR |
| | | | GLEWLGRTYYRSKWYNGYAVSMKSRITINPDTSKNQFSLQLNSV |
| | | | TPEDTAMYYCAREGVGATTGFDYWGQGTLVTVSS |
| 370 | ATX-P-562 | VH | QVQLQQSGPGLVKSSQTLSLTCAISGDTVSSNSAAWNWIRQSPSR |
| | | | GLEWLGRTFYRSRWSAAYAASVSSRITINPDTSKNQFSLQLTSVT |
| | | | PADTAVYYCAREGVGSSTGFDYWGQGTLVTVSS |
| 371 | ATX-P-566 | VH | QVQLQQSGPGLVKPSQTLSLTCAISGDSVSSNSAAWNWIRQSPSR |
| | | | GLEWLGRTYYRSKWFNNYSVSVKSRIAINPDTSKNQFSLQLNSVT |
| | | | PEDTAVYYCARDLGELLDWYFDLWGRGTLVTVSA |
| 372 | ATX-P-572 | VH | QVQLQQSGPGLVKPSQTLSLTCAISGDSVSSNSAAWNWIRQSPSR |
| | | | GLEWLGMTYYRSKWYSVFAVSVKSRITINTDTSKNQFSLQLNSV |
| | | | TPEDTAVYYCAREGGIVGATPFDYWGQGTLVTVSS |
| 373 | ATX-P-573 | VH | QVQLQQSGPGLVKPSQTLSLTCAISGDSVSSNSAAWNWIRQSPSR |
| | | | GLEWLGRTYYRSKLYSDYAVSVKSRITINPDTSRNQFSLQLNSVT |
| | | | PEDTAVYYCAREGGYTYGYDYWGQGTLVTVSS |
| 374 | ATX-P-581 | VH | QVQLQQSGPGLVKPSQTLSLTCAISGDSVSSNNAAWNWIRQSPSR |
| | | | GLEWLGRTYYRSKWYNAYAVSVKSRIUINPDTSRNQFSLQLNSVT |
| | | | PEDTAVYYCAREGGYIYGLDQWGQGTLVTVSS |
| 375 | ATX-P-587 | VH | QVQLQQSGPGLVKPSQTLSLTCAISGDSVSSNSAAWNWIRQSPSR |
| | | | GLEWLGKTYYRSKWYNYYALSVKSRITINPDTSKNQFSLHLNSV |
| | | | TPEDTAVYYCARDGDYYLPSPFDLWGHGTMVTVSS |
| 376 | ATX-P-592 | VH | QVQLQQSGPGLVKPSQTLSLTCAISGDSVSSNSAAWNWIRQSPSR |
| | | | GLEWLGRTYYRSKWYSDYPLSVKSRITINPDTSKNQFSLQLNSVT |
| | | | PEDTAVYYCARDSGIVETTPFDYWGQGTLVTVSS |
| 377 | ATX-P-594 | VH | QVQLQQSGPGLVKPSQTLSLTCAISGDSVSSNSASWNWIRQSPSR |
| | | | GLEWLGTTYYRSKWFNVYAVSVKSRITINPDTSKNQFSLHLNSVT |
| | | | PEDTAIYFCTRDRGDLLHWYFDLWGRGTLVTVSS |
| 378 | ATX-P-595 | VH | QVQLQQSGPGLVKPSQTLSLTCAISGDSVSSNSAAWNWIRQSPSR |
| | | | GLEWLGRTYYRSKWFNDYALSVKSRITINPDTSKNQFSLHLNSVT |
| | | | PEDTAVYYCAREPGIVAPGPFDYWGQGTLVTVSS |
| 379 | ATX-P-604 | VH | QVQLQQSGPGLVKPTQTLSLTCAISGDSVSSNSAAWNWIRQSPSR |
| | | | GLEWLGRTYYRSKWFNGYAVSVKSRITINPDASKNQFSLHLKSV |
| | | | TDEDTAVYYCATDRGELLHWYFDLWGRGTLVTVSS |
| 380 | ATX-P-610 | VH | QVQLQQSGPGLVKSSQTLSITCAISGDSVSSNSAAWNWIRQSPSR |
| | | | GLEWLGRTYYRSKWYNDYALSVKSRIIIKSDTSKNQFSLQLNSVT |
| | | | PEDTAVYYCAREPGIAAPGPFDYWGQGSLVTVSS |
| 381 | ATX-P-560 | VH - nucleotide | CAGGTACAGCTGCAGCAGTCAGGTCCAGGACTGGTGAAGCCC |
| | | | TCGCAGACCCTCTCACTCACCTGTGCCATTTCCGGGGACAGTG |
| | | | TCTCTAGCAACACTGCTGCTTGGAACTGGATCAGGCAGTCCCC |
| | | | ATCGAGAGGCCTTGAGTGGCTGGGAAGGACATACTACAGGTC |
| | | | CAAGTGGTATAATGATTATGCAGTATCTGTGAAAAGTCGAATA |
| | | | ACCATCAACCCAGACACATCCAAGAACCAGTTCTCCCTGCAGT |
| | | | TGAGCTCTGTGACTCCCCAGGACACGGCTATATATTACTGTGC |
| | | | GAGCGATAACTGGAACAACGGAGGCCCTGGGTTCGACCCCTG |
| | | | GGGCCAGGGAACCCTGGTCACCGTCTCCTCA |
| 382 | ATX-P-561 | VH - nucleotide | CAGGTACAGCTGCAGCAGTCAGGTCCAGGACTGGTGAAGCCC |
| | | | TCGCAGACCCTCTCACTCACCTGTGCCATCTCCGGGGACAGTG |
| | | | TCTCTAGCAACAGTGCTGCTTGGAACTGGATCAGGCAGTCCCC |
| | | | ATCGAGAGGCCTTGAGTGGCTGGGAAGGACATACTACAGGTC |
| | | | CAAGTGGTATAATGGTTATGCAGTCTCTATGAAAAGTCGAATA |
| | | | ACCATCAACCCAGACACATCCAAGAACCAGTTCTCCCTGCAGC |
| | | | TGAACTCTGTGACTCCCGAGGACACGGCTATGTATTATTGTGC |
| | | | AAGAGAGGGAGTGGGAGCTACTACAGGCTTTGACTACTGGGG |
| | | | CCAGGGAACCCTGGTCACCGTCTCCTCA |
| 383 | ATX-P-562 | VH - nucleotide | CAGGTACAGCTGCAGCAGTCAGGTCCAGGACTGGTGAAGTCC |
| | | | TCGCAGACCCTCTCACTCACCTGTGCCATCTCCGGGGACACTG |
| | | | TCTCTAGTAATAGTGCTGCTTGGAACTGGATCAGGCAGTCCCC |
| | | | ATCGAGAGGCCTTGAGTGGCTGGGAAGGACATTCTATAGGTCC |
| | | | AGGTGGTCTGCTGCTTATGCAGCGTCTGTGAGTAGTCGAATAA |
| | | | CCATCAACCCAGACACATCCAAGAACCAGTTCTCCCTGCAGCT |
| | | | GACCTCTGTGACTCCCGCGGACACGGCTGTGTATTACTGTGCA |
| | | | AGAGAGGGAGTAGGATCTTCTACAGGCTTTGACTACTGGGGCC |
| | | | AGGGAACCCTGGTCACCGTCTCCTCA |
| 384 | ATX-P-566 | VH - nucleotide | CAGGTACAGCTGCAGCAGTCAGGTCCAGGACTGGTGAAGCCC |
| | | | TCGCAGACCCTCTCACTCACCTGTGCCATCTCCGGGGACAGTG |
| | | | TCTCTAGCAACAGTGCTGCTTGGAACTGGATCAGGCAGTCCCC |
| | | | ATCGAGAGGCCTTGAGTGGCTGGGAAGGACATACTACAGGTC |
| | | | CAAGTGGTTTAATAATTATTCAGTTTCTGTGAAAAGTCGAATA |
| | | | GCCATTAACCCAGACACATCCAAGAACCAGTTCTCCCTTCAAC |
| | | | TGAACTCTGTGACTCCCGAGGACACGGCTGTGTATTACTGTGC |
| | | | AAGAGATCTGGGGGAGTTACTTGACTGGTACTTQGATCTCTGG |
| | | | GGCCGTGGCACCCTGGTCACTGTCTCCGCA |
| 385 | ATX-P-572 | VH - nucleotide | CAGGTACAGCTGCAGCAGTCAGGTCCAGGACTGGTGAAGCCC |
| | | | TCGCAGACCCTCTCACTCACCTGTGCCATCTCCGGGGACAGTG |
| | | | TCTCTAGCAACAGTGCTGCTTGGAACTGGATCAGGCAGTCCCC |
| | | | ATCGAGAGGCCTTGAGTGGCTGGGAATGACATACTACAGGTC |
| | | | CAAGTGGTATAGTGTTTTTGCAGTATCTGTGAAAAGTCGAATA |
| | | | ACCATCAATACAGACACATCCAAGAACCAGTTCTQCCTGCAGC |
| | | | TGAACTCTGTGACTCCCGAGGACACGGCTGTGTATTACTGTGC |
| | | | AAGAGAGGGGGGTATAGTGGGAGCCACCCCCTTTGACTACTG |
| | | | GGGCCAGGGAACCCTGGTCACCGTCTCTTCA |
| 386 | ATX-P-573 | VH - nucleotide | CAGGTACAGCTGCAGCAGTCAGGTCCAGGACTGGTGAAGCCC |
| | | | TCGCAGACCCTCTCACTCACCTGTGCCATCTCCGGGGACAGTG |
| | | | TCTCTAGCAACAGTGCTGCTTGGAACTGGATCAGGCAGTCCCC |
| | | | ATCGAGAGGCCTTGAGTGGCTGGGCAGGACATACTACAGGTC |
| | | | CAAGTTGTATAGTGATTATGCAGTATCTGTGAAAAGTCGAATA |
| | | | ACCATCAACCCAGACACATCCAGGAACCAGTTCTCCCTGCAGC |
| | | | TGAACTCTGTGACTCCCGAGGACACGGCTGTGTATTACTGTGC |
| | | | AAGAGAGGGGGGATACACCTATGGTTATGACTACTGGGGCCA |
| | | | GGGAACCCTGGTCACCGTCTCCTCA |
| 387 | ATX-P-581 | VH - nucleotide | CAGGTACAGCTGCAGCAGTCAGGTCCAGGACTGGTGAAGCCC |
| | | | TCGCAGACCCTCTCACTCACCTGTGCCATCTCCGGGGACAGTG |
| | | | TCTCTAGCAACAATGCTGCTTGGAACTGGATCAGGCAGTCCCC |
| | | | ATCGAGAGGCCTTGAGTGGCTGGGAAGGACGTACTACAGGTC |
| | | | CAAGTGGTATAATGCTTATGCAGTTTCTGTGAAAAGTCGAATT |
| | | | ATCATCAATCCAGACACATCCAGGAACCAGTTCTCCCTGCAAC |
| | | | TGAACTCTGTGACTCCCGAGGACACGGCTGTGTATTACTGTGC |
| | | | AAGAGAGGGTGGATACATCTATGGTCTTGACCAATGGGGCCA |
| | | | GGGAACCCTGGTCACCGTCTCCTCA |
| 388 | ATX-P-587 | VH - nucleotide | CAGGTGCAGCTGCAGCAGTCGGGTCCAGGACTGGTGAAGCCC |
| | | | TCGCAGACCCTCTCACTCACCTGTGCCATCTCCGGGGACAGTG |
| | | | TCTCTAGCAACAGTGCTGCTTGGAACTGGATCAGGCAGTCCCC |
| | | | ATCGAGAGGCCTTGAGTGGCTGGGAAAGACATACTACAGGTC |
| | | | CAAGTGGTATAATTATTATGCACTATCTGTGAAAAGTCGAATA |
| | | | ACCATCAACCCAGACACATCCAAGAACCAGTTCTQCCTGCACC |
| | | | TGAACTCTGTGACTCCCGAGGACACGGCTGTGTATTACTGTGC |
| | | | AAGAGACGGTGACTACTACCTTCCCTCTCCTTTTGATCTCTGGG |
| | | | GCCACGGGACAATGGTCACCGTCTCCTCA |
| 389 | ATX-P-592 | VH - nucleotide | CAGGTACAGCTGCAGCAGTCAGGTCCAGGACTGGTGAAGCCC |
| | | | TCGCAGACCCTCTCACTCACCTGTGCCATCTCCGGGGACAGTG |
| | | | TCTCTAGCAACAGTGCTGCTTGGAACTGGATCAGGCAGTCCCC |
| | | | ATCGAGAGGCCTTGAGTGGCTGGGAAGGACATACTACAGGTC |
| | | | CAAGTGGTATAGTGATTATCCACTATCTGTGAAAAGTCGAATA |
| | | | ACCATCAACCCCGACACATCCAAGAACCAGTTCTCCCTGCAGC |
| | | | TGAACTCTGTGACTCCCGAGGACACGGCTGTGTATTACTGTGC |
| | | | AAGAGACTCCGGTATAGTTGAAACCACGCCCTTTGACTATTGG |
| | | | GGCCAGGGAACCCTGGTCACCGTCTCCTCA |
| 390 | ATX-P-594 | VH - nucleotide | CAGGTACAGCTGCAGCAGTCAGGTCCAGGACTGGTGAAGCCC |
| | | | TCGCAGACCCTCTCACTCACCTGTGCCATCTCCGGGGACAGTG |
| | | | TCTCTAGTAACAGTGCTTCTTGGAACTGGATCAGGCAGTCCCC |
| | | | TTCGAGAGGCCTTGAGTGGCTGGGAACGACATACTACAGGTCC |
| | | | AAGTGGTTTAATGTTTATGCAGTCTCTGTGAAAAGTCGAATAA |
| | | | CCATCAACCCAGACACATCCAAGAACCAATTCTCCCTGCACCT |
| | | | GAACTCTGTGACTCCCGAGGACACGGCTATATATTTCTGTACA |
| | | | AGAGATQGGGGGGACCTACTCCACTGGTACTTCGATCTCTGGG |
| | | | GCCGTGGCACCCTGGTCACTGTCTCCTCA |
| 391 | ATX-P-595 | VH - nucleotide | CAGGTACAGCTGCAGCAGTCGGGTCCAGGACTGGTGAAGCCC |
| | | | TCGCAGACCCTCTCACTCACCTGTGCCATCTCCGGGGACAGTG |
| | | | TCTCTAGCAACAGTGCTGCTTGGAACTGGATCAGGCAGTCCCC |
| | | | ATCGAGAGGCCTTGAGTGGCTGGGAAGGACATACTACAGGTC |
| | | | CAAGTGGTTTAATGATTATGCACTATCTGTGAAAAGTCGAATA |
| | | | ACCATCAACCCAGACACATCCAAGAACCAGTTCTCCCTGCACC |
| | | | TGAACTCTGTGACTCCCGAGGACACGGCTGTGTATTACTGTGC |
| | | | AAGAGAACCTGGTATAGTAGCACCTGGCCCTTTTGACTACTGG |
| | | | GGCCAGGGAACCCTGGTCACCGTCTCCTCA |
| 392 | ATX-P-604 | VH - nucleotide | CAGGTACAGCTGCAGCAGTCAGGTCCAGGACTGGTGAAGCCC |
| | | | ACGCAGACCCTCTCACTCACCTGTGCCATCTCCGGGGACAGTG |
| | | | TCTCTAGCAACAGTGCTGCATGGAACTGGATCAGGCAGTCCCC |
| | | | ATCGAGAGGCCTTGAGTGGCTGGGGAGGACATACTACAGGTC |
| | | | CAAGTGGTTTAATGGTTATGCAGTATCTGTGAAAAGTCGAATA |
| | | | ACCATCAACCCCGACGCATCCAAGAACCAGTTCTCCCTGCACT |
| | | | TGAAGTCTGTGACTGACGAGGACACGGCTGTATATTACTGTGC |
| | | | AACAGATCGGGGGGAACTCCTCCACTGGTACTTCGATCTCTGG |
| | | | GGCCGTGGCACCCTGGTCACCGTCTCTTCA |
| 393 | ATX-P-610 | VH - nucleotide | CAGGTACAGCTGCAGCAGTCAGGTCCAGGACTGGTGAAGTCC |
| | | | TCGCAGACCCTCTCAATCACCTGTGCCATCTCCGGGGACAGTG |
| | | | TCTCTAGCAACAGTGCTGCTTGGAACTGGATCAGGCAGTCCCC |
| | | | ATCGAGAGGCCTTGAGTGGCTGGGAAGGACATACTACAGGTC |
| | | | CAAGTGGTATAATGATTATGCACTATCTGTGAAAAGTCGAATA |
| | | | ATCATCAAGTCAGACACATCCAAGAACCAGTTCTCCCTGCAGT |
| | | | TGAACTCTGTGACTCCCGAGGACACGGCTGTGTACTACTGTGC |
| | | | AAGAGAACCGGGTATAGCAGCACCTGGTCCATTTGACTACTGG |
| | | | GGCCAGGGATCCCTGGTCACCGTCTCCTCA |
| 394 | ATX-P-560 | VL | DIQMTQSPSSVSTSVGDRVTITCRASQGISSWLAWYQQKPGKAPK |
| | | | LLISAASSLQSGVPSRFSGSGSGTDFTLTISTLQPEDFASYYCQQAN |
| | | | SFPPTFGQGTRVEIK |
| 395 | ATX-P-561 | VL | DIQMTQSPSSLSASVGDRVTITCRASQGISSWLTWYQQKPGK.APK |
| | | | LLIYAASSLQGGVPSRFSGSGSGTDFTLTISSLQPEDFATYYCQQA |
| | | | NSFPPTFGQGTK VEIK |
| 396 | ATX-P-562 | VL | DIQMTQSPSSVSASIGDRVTITCRASKGISIWLAWYQQKPGKAPK |
| | | | VLISAASSLQSGAPSRFSGSGSGTDFTLTISSLQPEDFATYYCQQA |
| | | | NSFPPTFGQGTKVEIK |
| 397 | ATX-P-566 | VL | EIVLTQSPGTLSLSPGDRATLSCRASQSVRNSYVAWYQQKPGQAP |
| | | | RLLIYGASSRATGIPDRFSGSGSGTDFSLTIGRLEPEDFVVYYCQQ |
| | | | YGNSPLTFGGGTKVEIK |
| 398 | ATX-P-572 | VL | DIVMTQSPDSLAVSLGERATINCKSSQSVLYSSNNKNYLAWYQQ |
| | | | KPGQPPKLLIYWASPRESGVPDRFSGSGSGTDFTLTISSLQAEDVA |
| | | | VYYCQQYYSIPRTFGQGTKVEIK |
| 399 | ATX-P-573 | VL | DIQMTQSPSSVSASVGDRVTITCRASQGISSWLAWYQQKPGKAPK. |
| | | | VLIYAASSLQSGVPSRFSGSGSGTDFTLTISSLQPEDFATYYCQQA |
| | | | NSFPPTFGPGTKVDIK |
| 400 | ATX-P-581 | VL | DIQMTQSPSSVSASVGDRVTITCRASQGVNNWLAWYQQKPGKAP |
| | | | KLLIHAASSLLSGVPSRFSGSGSGTDFTLTITSLQPEDFATYYCQQ |
| | | | ANSFPITFGQGTRLEIK |
| 401 | ATX-P-587 | VL | DIVMTQSPDSLAVSLGERATINCKSSQSVLYSSNNKNYLAWYQQ |
| | | | KPGQPPKLLIYWASTRESGVPDRFSGSGSGTDFTLTISSLQAEDVA |
| | | | VYYCQQYYSTPYTFGQGTKLEIK |
| 402 | ATX-P-592 | VL | DIVMTQSPDSLAVSLGERATINCKSSQSVLYSSNNKNYLAWYQQ |
| | | | KPGQPPKLLIYWASNRESGVPDRFSGSGSGTDFTLTISSLQAEDVA |
| | | | VYYCQQYYSTPYTFGQGTKLEIK |
| 403 | ATX-P-594 | VL | EIVLTQSPGTLSLSPGERATLSCRASQSVSSSYLAWYQQKPGQSPR |
| | | | LLIYGASSRATGIPDRFSGSGSGTDFTLTISRLEPEDFAVYYCQQY |
| | | | GRSVTFGGGTKLEIK |
| 404 | ATX-P-595 | VL | DIVMTQSPDSLAVSLGERATINCKSSQSVLYSSNNKNYLAWYQQ |
| | | | KPGQPPKLLIYWASTRESGVPDRFSGSGSGTDFTLTISSLQAEDVA |
| | | | VYYCQQYYSTPYTFGQGTKLEIK |
| 405 | ATX-P-604 | VL | EIVLTQSPDTLSLSPGERATLSCRASQTIRSSYLAWYQLKPGQAPR |
| | | | LLIYGASSRATGIPDRESGSGSGTDFTLSISRLEPEDFAVYYCQQY |
| | | | GRSIIFGGGTKVEIK |
| 406 | ATX-P-610 | VL | DIQMTQSPSSVSASVGDRVTITCRASQGISSWLAWYQQKPGKAPK |
| | | | LLIYAASSLQSGVPSRFSGSGSGTDFTLTISSLQPEDFATYYCQQN |
| | | | YRTPITFGQGTRLEIK |
| 407 | ATX-P-581 | VL - nucleotide | GACATCCAGATGACCCAGTCTCCATCTTCCGTGTCTACATCTGT |
| | | | TGGAGACAGAGTCACCATCACTTGTCGGGCGAGTCAGGGTATT |
| | | | AGCAGCTGGTTAGCCTGGTATCAGCAGAAACCAGGGAAAGCC |
| | | | CCTAAGCTCCTGATCTCTGCTGCATCCAGTTTGCAAAGTGGGG |
| | | | TCCCATCAAGGTTCAGCGGCAGTGGATCTGGGACAGATTTCAC |
| | | | TCTCACCATCAGCACCCTGCAGCCTGAAGACTTTGCATCTTAC |
| | | | TATTGTCAACAGGCTAACAGTTTCCCTCCGACGTTQGGCCAAG |
| | | | GGACCAGGGTGGAAATCAAA |
| 408 | ATX-P-561 | VL - nucleotide | GACATCCAGATGACCCAGTCTCCATCTTCCCTGTCTGCATCTGT |
| | | | CGGAGACAGAGTCACCATCACTTGTCGGGCGAGTCAGGGTATT |
| | | | AGCAGCTGGTTAACCTGGTATCAGCAGAAACCAGGGAAAGCC |
| | | | CCTAAGCTCCTGATCTATGCTGCATCCAGTTTGCAAGGTGGGG |
| | | | TCCCATCAAGATTCAGCGGCAGTGGATCTGGGACAGATTTCAC |
| | | | TCTCACCATCAGCAGCCTGCAGCCTGAAGATTTTGCAACTTAC |
| | | | TATTGTCAACAGGCTAACAGTTTCCCTCCGACGTTQGGCCAAG |
| | | | GGACCAAGGTGGAAATCAAA |
| 409 | ATX-P-573 | VL - nucleotide | GACATCCAGATGACCCAGTCTCCATCTTCCGTGTCTGCATCTAT |
| | | | AGGAGACAGAGTCACCATCACTTGTCGGGCGAGTAAGGGAAT |
| | | | TAGCATCTGGTTAGCCTGGTATCAGCAGAAACCAGGGAAAGC |
| | | | CCCTAAGGTCCTGATCTCTGCTGCATCCAGTTTGCAAAGTGGG |
| | | | GCCCCATCAAGGTTCAGCGGCAGTGGATCTGGGACAGATTTCA |
| | | | CTCTCACCATCAGCAGCCTGCAGCCTGAAGATTTTGCAACTTA |
| | | | CTATTGTCAACAGGCTAACAGTTTCCCTCCGACGTTCGGCCAA |
| | | | GGGACCAAGGTGGAGATCAAA |
| 410 | ATX-P-610 | VL - nucleotide | GAAATTGTGTTGACGCAGTCTCCAGGCACCCTGTCTTTGTCTCC |
| | | | AGGGGACAGAGCCACCCTCTCATGCAGGGCCAGTCAGAGTGT |
| | | | TAGGAACAGTTACGTAGCCTGGTACCAGCAGAAACCTGGCCA |
| | | | GGCTCCCAGGCTCCTCATCTATGGTGCATCCAGCAGGGCCACT |
| | | | GGCATCCCAGACAGGTTCAGTGGCAGTGGGTCTGGGACAGAC |
| | | | TTCAGTCTCACCATCGGCAGACTGGAGCCTGAGGATTTTGTAG |
| | | | TGTATTATTGTCAGCAATACGGTAACTCACCTCTCACTTTCGGC |
| | | | GGAGGGACCAAGGTGGAGATCAAA |
| 411 | ATX-P-560 | VL - nucleotide | GACATCGTGATGACCCAGTCTCCAGACTCCCTGGCTGTGTCTC |
| | | | TGGGCGAGAGGGCCACCATCAACTGCAAGTCCAGCCAGAGTG |
| | | | TTTTATACAGCTCCAACAATAAGAACTACTTAGCTTGGTACCA |
| | | | GCAGAAACCTGGACAGCCTCCTAAGCTGCTCATTTACTGGGCA |
| | | | TCTCCCCGGGAATCCGGGGTCCCTGACCGGTTCAGTGGCAGCG |
| | | | GGTCTGGGACAGATTTCACTCTCACCATCAGCAGCCTGCAGGC |
| | | | TGAAGATGTGGCAGTTTATTATTGTCAGCAATATTATAGTATT |
| | | | CCTCGGACGTTCGGCCAAGGGACCAAGGTGGAAATCAAA |
| 412 | ATX-P-562 | VL - nucleotide | GACATCCAGATGACCCAGTCTCCATCTTCCGTGTCTGCATCTGT |
| | | | AGGAGACAGAGTCACCATCACTTGTCGGGCGAGTCAGGGTAT |
| | | | TAGCAGCTGGTTAGCCTGGTATCAGCAGAAACCAGGGAAAGC |
| | | | CCCTAAGGTCCTGATCTATGCTGCATCCAGTTTGCAAAGTGGG |
| | | | GTCCCATCAAGGTTCAGCGGCAGTGGATCTGGGACAGATTTCA |
| | | | CTCTCACCATCAGCAGCCTGCAGCCTGAAGATTTTGCAACTTA |
| | | | CTATTGTCAACAGGCTAACAGTTTCCCTCCCACTTTCGGCCCTG |
| | | | GGACCAAAGTGGATATCAAA |
| 413 | ATX-P-566 | VL - nucleotide | GACATCCAGATGACTCAGTCTCCATCTTCCGTGTCTGCATCTGT |
| | | | AGGAGACAGAGTCACCATCACTTGTCGGGCGAGTCAGGGTGT |
| | | | TAACAACTGGTTAGCCTGGTATCAGCAGAAACCAGGGAAAGC |
| | | | CCCTAAGCTCCTGATCCATGCTGCATCCAGTTTGCTAAGTGGG |
| | | | GTCCCATCAAGGTTCAGCGGCAGTGGATCTGGGACAGATTTCA |
| | | | CGCTCACCATCACCAGCCTGCAGCCTGAAGATTTTGCAACTTA |
| | | | CTATTGTCAACAGGCTAACAGTTTCCCCATCACCTTCGGCCAA |
| | | | GGGACACGACTGGAAATTAAA |
| 414 | ATX-P-604 | VL - nucleotide | GACATCGTGATGACCCAGTCTCCAGACTCCCTGGCTGTGTCTC |
| | | | TGGGCGAGAGGGCCACCATCAACTGCAAGTCCAGCCAGAGTG |
| | | | TTTTATACAGCTCCAACAATAAGAACTACTTAGCTTGGTACCA |
| | | | GCAGAAACCAGGACAGCCTCCTAAGCTGCTCATTTACTGGGCA |
| | | | TCTACCCGGGAATCCGGGGTCCCTGACCGATTCAGTGGCAGCG |
| | | | GGTCTGGGACAGATTTCACTCTCACCATCAGCAGCCTGCAGGC |
| | | | TGAAGATGTGGCAGTTTATTACTGTCAGCAATATTATAGTACT |
| | | | CCGTACACTTTTGGCCAGGGGACCAAGCTGGAGATCAAA |
| 415 | ATX-P-594 | VL - nucleotide | GACATCGTGATGACCCAGTCTCCAGACTCCCTGGCTGTGTCTC |
| | | | TGGGCGAGAGGGCCACCATCAACTGCAAGTCCAGCCAGAGTG |
| | | | TTTTATACAGCTCCAACAATAAGAACTACTTAGCTTGGTACCA |
| | | | GCAGAAACCAGGACAGCCTCCTAAGCTGCTCATTTACTGGGCA |
| | | | TCTAACCGGGAATCCGGGGTCCCTGACCGATTCAGTGGCTCCG |
| | | | GGTCTGGGACAGATTTCACTCTCACCATCAGCAGCCTGCAGGC |
| | | | TGAAGATGTGGCAGTTTATTACTGTCAGCAATATTATAGTACT |
| | | | CCGTACACTTTTGGCCAGGGGACCAAGCTGGAGATCAAA |
| 416 | ATX-P-572 | VL - nucleotide | GAAATTGTGTTGACGCAGTCTCCAGGCACCCTGTCTTTGTCTCC |
| | | | AGGGGAGAGAGCCACCCTCTCCTGCAGGGCCAGTCAGAGTGT |
| | | | TAGCAGCAGCTACTTAGCCTGGTACCAGCAGAAACCTGGCCA |
| | | | GTCTCCCAGGCTCCTCATCTATGGTGCATCCAGCAGGGCCACT |
| | | | GGCATCCCAGACAGGTTCAGTGGCAGTGGGTCTGGGACAGAC |
| | | | TTCACTCTCACCATCAGCAGACTGGAGCCTGAGGATTTTGCAG |
| | | | TATATTACTGTCAGCAGTATGGTAGGTCAGTCACTTTCGGCGG |
| | | | AGGGACCAAGCTGGAAATCAAA |
| 417 | ATX-P-587 | VL - nucleotide | GACATCGTGATGACCCAGTCTCCAGACTCCCTGGCTGTGTCTC |
| | | | TGGGCGAGAGGGCCACCATCAACTGCAAGTCCAGCCAGAGTG |
| | | | TTTTATACAGCTCCAACAATAAGAACTACTTAGCTTGGTACCA |
| | | | GCAGAAACCAGGACAGCCTCCTAAGCTGCTCATTTACTGGGCA |
| | | | TCTACCCGGGAATCCGGGGTCCCTGACCGATTCAGTGGCAGCG |
| | | | GGTCTGGGACAGATTTCACTCTCACCATCAGCAGCCTGCAGGC |
| | | | TGAAGATGTGGCAGTTTATTACTGTCAGCAATATTATAGTACT |
| | | | CCGTACACTTTTGGCCAGGGGACCAAGCTGGAAATCAAA |
| 418 | ATX-P-592 | VL - nucleotide | GAAATAGTGCTGACTCAGTCTCCAGACACCCTATCTTTGTCTC |
| | | | CAGGGGAAAGAGCCACCCTCTCCTGCAGGGCCAGTCAGACTA |
| | | | TTAGGAGCAGCTACTTAGCCTGGTACCAGCTGAAACCTGGCCA |
| | | | GGCTCCCAGGCTCCTCATCTATGGTGCATCCAGCAGGGCCACT |
| | | | GGCATCCCAGACAGGTTCAGTGGCAGTGGGTCTGGGACAGAC |
| | | | TTCACTCTCTCCATCAGCAGACTGGAGCCTGAAGATTTTGCAG |
| | | | TGTATTACTGTCAACAGTATGGCCGCTCAATCATTTTCGGCGG |
| | | | AGGGACCAAAGTGGAAATCAAA |
| 419 | ATX-P-595 | VL - nucleotide | GACATCCAGATGACCCAGTCTCCATCTTCCGTGTCTGCATCTGT |
| | | | AGGAGACAGAGTCACCATCACTTGTCGGGCGAGTCAGGGTAT |
| | | | TAGCAGCTGGTTAGCCTGGTATCAGCAGAAACCAGGGAAAGC |
| | | | CCCTAAGCTCCTGATCTATGCTGCATCCAGTTTGCAAAGTGGG |
| | | | GTCCCATCAAGGTTCAGCGGCAGTGGATCTGGGACAGATTTCA |
| | | | CTCTCACCATCAGCAGCCTGCAGCCTGAAGATTTTGCAACTTA |
| | | | CTACTGTCAACAGAATTACAGAACCCCGATCACCTTCGGCCAA |
| | | | GGGACACGACTGGAGATTAAA |

TABLE 5
Anti-CFHR4 antibodies (HC IgG1 Fc and LC Kappa sequences)

| SEQ ID | Antibody | | |
|---|---|---|---|
| NO: | Name: | Descriptor: | Sequence: |
| 420 | ATX-P-583 | HC IgG1 Fc | QVQLVQSGAEVKKPGASVKVSCKASGYTFTTYGISWVRQAPGQGLE |
| | | | WMGWISAYSGNTHYAQSLQDRVTMTTDTSTSTAYMEVRSLRSDDTA |
| | | | VYYCAKDEGYCSSTGCLNLFDPWGQGTLVTVSSASTKGPSVFPLAPS |
| | | | SKSTSGGTAALGCLVKDYFPEPVTVSWNSGALTSGVHTFPAVLQSSG |
| | | | LYSLSSVVTVPSSSLGTQTYICNVNHKPSNTKVDKRVEPKSCDKTHTC |
| | | | PPCPAPELLGGPSVFLFPPKPKDTLMISRTPEVTCVVVDVSHEDPEVKF |
| | | | NWYVDGVEVHNAKTKPREEQYASTYRVVSVLTVLHQDWLNGKEYK |
| | | | CKVSNKALPAPIEKTISKAKGQPREPQVYTLPPSREEMTKNQVSLTCL |
| | | | VKGFYPSDIAVEWESNGQPENNYKTTPPVLDSDGSFFLYSKLTVDKSR |
| | | | WQQGNVFSCSVMHEALHNHYTQKSLSLSPG |
| 421 | ATX-P-591 | HC IgG1 Fc | QVQLVQSGAEVKKPGASVKVSCKASGYTFTSYGISWVRQAPGQGLE |
| | | | WMGWISAYNGNTNYAQKLQGRVTMTTDTSTSTAYMELRSLRSDDT |
| | | | AIYYCTRDLRGTAFDYWGQGTPVTVSSASTKGPSVFPLAPSSKSTSGG |
| | | | TAALGCLVKDYFPEPVTVSWNSGALTSGVHTFPAVLQSSGLYSLSSV |
| | | | VTVPSSSLGTQTYICNVNHKPSNTKVDKRVEPKSCDKTHTCPPCPAPE |
| | | | LLGGPSVFLFPPKPKDTLMISRTPEVTCVVVDVSHEDPEVKFNWYVD |
| | | | GVEVHNAKTKPREEQYASTYRVVSVLTVLHQDWLNGKEYKCKVSN |
| | | | KALPAPIEKTISKAKGQPREPQVYTLPPSREEMTKNQVSLTCLVKGFY |
| | | | PSDIAVEWESNGQPENNYKTTPPVLDSDGSFFLYSKLTVDKSRWQQG |
| | | | NVFSCSVMHEALHNHYTQKSLSLSPG |
| 422 | ATX-P-602 | HC IgG1 Fc | QVQLVQSGAEVKKPGASVKVSCKASGYTFTGYYMHWVRQAPGQGL |
| | | | EWMGWINPNSGGTNYAKKFQGRVTMTRDTSISTAYMELSRLRSDDT |
| | | | AVYYCARGDTIFRLVTPLDYWGQGTLVTVSSASTKGPSVFPLAPSSKS |
| | | | TSGGTAALGCLVKDYFPEPVTVSWNSGALTSGVHTFPAVLQSSGLYS |
| | | | LSSVVTVPSSSLGTQTYICNVNHKPSNTKVDKRVEPKSCDKTHTCPPC |
| | | | PAPELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVYTLPPSREEMTKNQVSLTCLVK |
| | | | GFYPSDIAVEWESNGQPENNYKTTPPVLDSDGSFFLYSKLTVDKSRW |
| | | | QQGNVFSCSVMHEALHNHYTQKSLSLSPG |
| 423 | ATX-P-597 | HC IgG1 Fc | QVQLVQSGAEVKKPGASVKVSCKASGYTFTNYYMQWVRQAPGQGL |
| | | | EWLGIIDPSGGSTTYAQKFQGRVTMTRDTSTSTVHMELRSLRSEDTA |
| | | | VYYCARDEGTDRPGWGYWGQGTLVTVSSASTKGPSVFPLAPSSKSTS |
| | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCPA |
| | | | PELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 424 | ATX-P-601 | HC IgG1 Fc | QVQLVQSGAEVKKPGASVKVSCKASGYTFTSYYMHWVRQAPGQGL |
| | | | EWMGIINPSGGSTNYAQKFQGRLTMTRDTSTNTVYMELSSLRSEDTA |
| | | | VYYCATDQSGGMDVWGKGTTVTVSSASTKGPSVFPLAPSSKSTSGGT |
| | | | AALGCLVKDYFPEPVTVSWNSGALTSGVHTFPAVLQSSGLYSLSSVV |
| | | | TVPSSSLGTQTYICNVNHKPSNTKVDKRVEPKSCDKTHTCPPCPAPEL |
| | | | LGGPSVFLFPPKPKDTLMISRTPEVTCVVVDVSHEDPEVKFNWYVDG |
| | | | VEVHNAKTKPREEQYASTYRVVSVLTVLHQDWLNGKEYKCKVSNK |
| | | | ALPAPIEKTISKAKGQPREPQVYTLPPSREEMTKNQVSLTCLVKGFYPS |
| | | | DIAVEWESNGQPENNYKTTPPVLDSDGSFFLYSKLTVDKSRWQQGNV |
| | | | FSCSVMHEALHNHYTQKSLSLSPG |
| 425 | ATX-P-607 | HC IgG1 Fc | QVQLVQSGAEVKKPGASVKVSCKASGYTFTSYYMHWVRQAPGQGL |
| | | | EWMGIINPSGGSTSYAQKFQGRVTMTRDTSTNTVYMELSSLRSEDTA |
| | | | VYYCARDPEYSSAFDIWGQGTMVTVSSASTKGPSVFPLAPSSKSTSGG |
| | | | TAALGCLVKDYFPEPVTVSWNSGALTSGVHTFPAVLQSSGLYSLSSV |
| | | | VTVPSSSLGTQTYICNVNHKPSNTKVDKRVEPKSCDKTHTCPPCPAPE |
| | | | LLGGPSVFLFPPKPKDTLMISRTPEVTCVVVDVSHEDPEVKFNWYVD |
| | | | GVEVHNAKTKPREEQYASTYRVVSVLTVLHQDWLNGKEYKCKVSN |
| | | | KALPAPIEKTISKAKGQPREPQVYTLPPSREEMTKNQVSLTCLVKGFY |
| | | | PSDIAVEWESNGQPENNYKTTPPVLDSDGSFFLYSKLTVDKSRWQQG |
| | | | NVFSCSVMHEALHNHYTQKSLSLSPG |
| 426 | ATX-P-583 | HC IgG1 Fc- | CAGGTTCAGCTGGTGCAGTCTGGAGCTGAGGTGAAGAAGCCTGGG |
| | | nucleotide | GCCTCAGTGAAGGTCTCCTGCAAGGCTTCTGGTTACACCTTTACCA |
| | | | CCTATGGTATCAGCTGGGTGCGACAGGCCCCTGGACAAGGGCTTG |
| | | | AGTGGATGGGATGGATCAGCGCTTACAGTGGTAACACACACTATG |
| | | | CACAGAGCCTCCAGGACAGAGTCACCATGACCACAGACACATCCA |
| | | | CGAGTACAGCCTACATGGAGGTGAGGAGCCTGAGATCTGACGACA |
| | | | CGGCCGTGTATTACTGTGCGAAAGATGAGGGATATTGTAGTAGTA |
| | | | CCGGCTGCTTAAACTTGTTCGACCCCTGGGGCCAGGGAACCCTGGT |
| | | | CACCGTCTCCTCAGCTAGCACTAAAGGGCCTTCTGTATTTCCCTTG |
| | | | GCCCCGTCCAGCAAATCGACCTCGGGAGGGACAGCCGCCCTGGGT |
| | | | TGCCTTGTGAAAGATTATTTCCCTGAGCCAGTTACCGTAAGTTGGA |
| | | | ACAGTGGGGCGCTGACAAGTGGTGTGCACACGTTTCCTGCCGTCCT |
| | | | GCAATCATCGGGCTTGTATAGCCTCAGCTCTGTGGTCACTGTCCCA |
| | | | AGTTCATCGCTGGGCACTCAGACGTATATTTGCAATGTGAACCACA |
| | | | AACCTTCAAATACAAAAGTGGATAAACGCGTAGAACCGAAATCGT |
| | | | GTGATAAAACTCACACATGCCCGCCATGCCCGGCACCTGAACTGC |
| | | | TTGGTGGTCCCAGCGTGTTCCTGTTCCCGCCGAAGCCTAAAGATAC |
| | | | TCTAATGATCAGCCGTACGCCAGAGGTGACATGTGTCGTGGTTGA |
| | | | CGTGTCCCACGAAGATCCCGAAGTTAAGTTCAATTGGTATGTTGAT |
| | | | GGTGTAGAGGTACACAATGCTAAGACTAAACCTCGCGAGGAGCAG |
| | | | TACGCCTCGACCTATCGTGTCGTGAGCGTTCTGACCGTCCTTCACC |
| | | | AAGATTGGCTTAACGGCAAAGAATATAAGTGCAAGGTAAGCAATA |
| | | | AAGCACTTCCGGCCCCAATCGAGAAAACCATTTCCAAGGCCAAAG |
| | | | GTCAACCAAGAGAACCCCAGGTGTATACTCTTCCGCCTTCTCGTGA |
| | | | GGAAATGACTAAAAATCAAGTATCCCTTACGTGTCTGGTTAAAGG |
| | | | TTTTTATCCTAGCGATATTGCTGTTGAATGGGAATCGAACGGTCAG |
| | | | CCGGAGAATAATTATAAAACAACGCCACCCGTCCTGGATAGCGAC |
| | | | GGCTCATTTTTTCTGTATAGCAAACTGACTGTAGATAAATCACGGT |
| | | | GGCAGCAGGGCAATGTATTCAGTTGCTCCGTTATGCATGAAGCGTT |
| | | | ACATAATCACTACACGCAGAAATCTCTTAGTCTTTCACCCGGT |
| 427 | ATX-P-591 | HC IgG1 Fc- | CAGGTTCAGCTGGTGCAGTCTGGAGCTGAGGTGAAGAAGCCTGGG |
| | | nucleotide | GCCTCAGTGAAGGTCTCCTGCAAGGCTTCTGGTTACACCTTTACCA |
| | | | GCTATGGTATCAGCTGGGTGCGACAGGCCCCTGGACAAGGGCTTG |
| | | | AGTGGATGGGATGGATCAGCGCTTACAATGGTAACACAAACTATG |
| | | | CACAGAAGCTCCAGGGCAGAGTCACCATGACCACAGACACATCCA |
| | | | CGAGCACAGCCTACATGGAGCTGAGGAGCCTGAGATCTGACGACA |
| | | | CTGCCATTTATTACTGTACGAGAGATCTACGTGGAACTGCCTTTGA |
| | | | CTACTGGGGCCAGGGAACCCCGGTCACTGTCTCCTCAGCTAGCACT |
| | | | AAAGGGCCTTCTGTATTTCCCTTGGCCCCGTCCAGCAAATCGACCT |
| | | | CGGGAGGGACAGCCGCCCTGGGTTGCCTTGTGAAAGATTATTTCC |
| | | | CTGAGCCAGTTACCGTAAGTTGGAACAGTGGGGCGCTGACAAGTG |
| | | | GTGTGCACACGTTTCCTGCCGTCCTGCAATCATCGGGCTTGTATAG |
| | | | CCTCAGCTCTGTGGTCACTGTCCCAAGTTCATCGCTGGGCACTCAG |
| | | | ACGTATATTTGCAATGTGAACCACAAACCTTCAAATACAAAAGTG |
| | | | GATAAACGCGTAGAACCGAAATCGTGTGATAAAACTCACACATGC |
| | | | CCGCCATGCCCGGCACCTGAACTGCTTGGTGGTCCCAGCGTGTTCC |
| | | | TGTTCCCGCCGAAGCCTAAAGATACTCTAATGATCAGCCGTACGCC |
| | | | AGAGGTGACATGTGTCGTGGTTGACGTGTCCCACGAAGATCCCGA |
| | | | AGTTAAGTTCAATTGGTATGTTGATGGTGTAGAGGTACACAATGCT |
| | | | AAGACTAAACCTCGCGAGGAGCAGTACGCCTCGACCTATCGTGTC |
| | | | GTGAGCGTTCTGACCGTCCTTCACCAAGATTGGCTTAACGGCAAA |
| | | | GAATATAAGTGCAAGGTAAGCAATAAAGCACTTCCGGCCCCAATC |
| | | | GAGAAAACCATTTCCAAGGCCAAAGGTCAACCAAGAGAACCCCA |
| | | | GGTGTATACTCTTCCGCCTTCTCGTGAGGAAATGACTAAAAATCAA |
| | | | GTATCCCTTACGTGTCTGGTTAAAGGTTTTTATCCTAGCGATATTG |
| | | | CTGTTGAATGGGAATCGAACGGTCAGCCGGAGAATAATTATAAAA |
| | | | CAACGCCACCCGTCCTGGATAGCGACGGCTCATTTTTTCTGTATAG |
| | | | CAAACTGACTGTAGATAAATCACGGTGGCAGCAGGGCAATGTATT |
| | | | CAGTTGCTCCGTTATGCATGAAGCGTTACATAATCACTACACGCAG |
| | | | AAATCTCTTAGTCTTTCACCCGGT |
| 428 | ATX-P-602 | HC IgG1 Fc- | CAGGTGCAGCTGGTGCAATCTGGGGCTGAGGTGAAGAAGCCTGGG |
| | | nucleotide | GCCTCAGTGAAGGTCTCCTGCAAGGCTTCTGGATACACCTTCACCG |
| | | | GCTACTATATGCACTGGGTGCGACAGGCCCCTGGACAAGGACTTG |
| | | | AGTGGATGGGATGGATCAACCCTAACAGTGGTGGCACAAACTATG |
| | | | CAAAGAAGTTTCAGGGCAGGGTCACCATGACCAGGGACACGTCCA |
| | | | TCAGCACAGCCTACATGGAGTTGAGCAGGCTGAGATCTGACGACA |
| | | | CGGCCGTGTATTACTGTGCGAGAGGGGATACGATTTTTCGACTGGT |
| | | | TACCCCTCTTGACTACTGGGGCCAGGGAACCCTGGTCACCGTCTCC |
| | | | TCAGCTAGCACTAAAGGGCCTTCTGTATTTCCCTTGGCCCCGTCCA |
| | | | GCAAATCGACCTCGGGAGGGACAGCCGCCCTGGGTTGCCTTGTGA |
| | | | AAGATTATTTCCCTGAGCCAGTTACCGTAAGTTGGAACAGTGGGG |
| | | | CGCTGACAAGTGGTGTGCACACGTTTCCTGCCGTCCTGCAATCATC |
| | | | GGGCTTGTATAGCCTCAGCTCTGTGGTCACTGTCCCAAGTTCATCG |
| | | | CTGGGCACTCAGACGTATATTTGCAATGTGAACCACAAACCTTCA |
| | | | AATACAAAAGTGGATAAACGCGTAGAACCGAAATCGTGTGATAAA |
| | | | ACTCACACATGCCCGCCATGCCCGGCACCTGAACTGCTTGGTGGTC |
| | | | CCAGCGTGTTCCTGTTCCCGCCGAAGCCTAAAGATACTCTAATGAT |
| | | | CAGCCGTACGCCAGAGGTGACATGTGTCGTGGTTGACGTGTCCCA |
| | | | CGAAGATCCCGAAGTTAAGTTCAATTGGTATGTTGATGGTGTAGA |
| | | | GGTACACAATGCTAAGACTAAACCTCGCGAGGAGCAGTACGCCTC |
| | | | GACCTATCGTGTCGTGAGCGTTCTGACCGTCCTTCACCAAGATTGG |
| | | | CTTAACGGCAAAGAATATAAGTGCAAGGTAAGCAATAAAGCACTT |
| | | | CCGGCCCCAATCGAGAAAACCATTTCCAAGGCCAAAGGTCAACCA |
| | | | AGAGAACCCCAGGTGTATACTCTTCCGCCTTCTCGTGAGGAAATG |
| | | | ACTAAAAATCAAGTATCCCTTACGTGTCTGGTTAAAGGTTTTTATC |
| | | | CTAGCGATATTGCTGTTGAATGGGAATCGAACGGTCAGCCGGAGA |
| | | | ATAATTATAAAACAACGCCACCCGTCCTGGATAGCGACGGCTCAT |
| | | | TTTTTCTGTATAGCAAACTGACTGTAGATAAATCACGGTGGCAGCA |
| | | | GGGCAATGTATTCAGTTGCTCCGTTATGCATGAAGCGTTACATAAT |
| | | | CACTACACGCAGAAATCTCTTAGTCTTTCACCCGGT |
| 429 | ATX-P-597 | HC IgG1 Fc- | CAGGTGCAGCTGGTGCAATCTGGAGCTGAGGTGAAGAAGCCTGGG |
| | | nucleotide | GCCTCAGTGAAGGTTTCCTGCAAGGCATCTGGATACACCTTCACCA |
| | | | ACTACTATATGCAGTGGGTGCGACAGGCCCCTGGACAAGGGCTTG |
| | | | AGTGGCTGGGAATAATCGACCCTAGTGGTGGTAGCACAACCTATG |
| | | | CACAGAAGTTCCAGGGCAGAGTCACCATGACCAGGGACACGTCCA |
| | | | CGAGCACAGTCCACATGGAGCTGAGAAGTCTGAGATCTGAGGACA |
| | | | CGGCCGTCTATTATTGCGCGAGAGATGAGGGAACAGATCGTCCCG |
| | | | GGTGGGGCTACTGGGGCCAGGGAACCCTGGTCACTGTCTCCTCAG |
| | | | CTAGCACTAAAGGGCCTTCTGTATTTCCCTTGGCCCCGTCCAGCAA |
| | | | ATCGACCTCGGGAGGGACAGCCGCCCTGGGTTGCCTTGTGAAAGA |
| | | | TTATTTCCCTGAGCCAGTTACCGTAAGTTGGAACAGTGGGGCGCTG |
| | | | ACAAGTGGTGTGCACACGTTTCCTGCCGTCCTGCAATCATCGGGCT |
| | | | TGTATAGCCTCAGCTCTGTGGTCACTGTCCCAAGTTCATCGCTGGG |
| | | | CACTCAGACGTATATTTGCAATGTGAACCACAAACCTTCAAATAC |
| | | | AAAAGTGGATAAACGCGTAGAACCGAAATCGTGTGATAAAACTCA |
| | | | CACATGCCCGCCATGCCCGGCACCTGAACTGCTTGGTGGTCCCAGC |
| | | | GTGTTCCTGTTCCCGCCGAAGCCTAAAGATACTCTAATGATCAGCC |
| | | | GTACGCCAGAGGTGACATGTGTCGTGGTTGACGTGTCCCACGAAG |
| | | | ATCCCGAAGTTAAGTTCAATTGGTATGTTGATGGTGTAGAGGTACA |
| | | | CAATGCTAAGACTAAACCTCGCGAGGAGCAGTACGCCTCGACCTA |
| | | | TCGTGTCGTGAGCGTTCTGACCGTCCTTCACCAAGATTGGCTTAAC |
| | | | GGCAAAGAATATAAGTGCAAGGTAAGCAATAAAGCACTTCCGGCC |
| | | | CCAATCGAGAAAACCATTTCCAAGGCCAAAGGTCAACCAAGAGAA |
| | | | CCCCAGGTGTATACTCTTCCGCCTTCTCGTGAGGAAATGACTAAAA |
| | | | ATCAAGTATCCCTTACGTGTCTGGTTAAAGGTTTTTATCCTAGCGA |
| | | | TATTGCTGTTGAATGGGAATCGAACGGTCAGCCGGAGAATAATTA |
| | | | TAAAACAACGCCACCCGTCCTGGATAGCGACGGCTCATTTTTTCTG |
| | | | TATAGCAAACTGACTGTAGATAAATCACGGTGGCAGCAGGGCAAT |
| | | | GTATTCAGTTGCTCCGTTATGCATGAAGCGTTACATAATCACTACA |
| | | | CGCAGAAATCTCTTAGTCTTTCACCCGGT |
| 430 | ATX-P-601 | HC IgG1 Fc- | CAGGTGCAGCTGGTGCAATCTGGGGCTGAGGTGAAGAAGCCTGGG |
| | | nucleotide | GCCTCAGTGAAGGTTTCCTGCAAGGCATCTGGATACACCTTCACCA |
| | | | GCTACTATATGCACTGGGTGCGACAGGCCCCTGGACAAGGGCTTG |
| | | | AGTGGATGGGAATAATCAACCCTAGTGGTGGTAGCACAAACTACG |
| | | | CACAGAAGTTCCAGGGCAGACTCACCATGACCAGGGACACGTCCA |
| | | | CGAACACAGTCTACATGGAACTGAGTAGCCTGAGATCTGAGGACA |
| | | | CGGCCGTGTATTACTGTGCGACAGATCAAAGTGGGGGGATGGACG |
| | | | TCTGGGGCAAAGGGACCACGGTCACCGTCTCCTCAGCTAGCACTA |
| | | | AAGGGCCTTCTGTATTTCCCTTGGCCCCGTCCAGCAAATCGACCTC |
| | | | GGGAGGGACAGCCGCCCTGGGTTGCCTTGTGAAAGATTATTTCCCT |
| | | | GAGCCAGTTACCGTAAGTTGGAACAGTGGGGCGCTGACAAGTGGT |
| | | | GTGCACACGTTTCCTGCCGTCCTGCAATCATCGGGCTTGTATAGCC |
| | | | TCAGCTCTGTGGTCACTGTCCCAAGTTCATCGCTGGGCACTCAGAC |
| | | | GTATATTTGCAATGTGAACCACAAACCTTCAAATACAAAAGTGGA |
| | | | TAAACGCGTAGAACCGAAATCGTGTGATAAAACTCACACATGCCC |
| | | | GCCATGCCCGGCACCTGAACTGCTTGGTGGTCCCAGCGTGTTCCTG |
| | | | TTCCCGCCGAAGCCTAAAGATACTCTAATGATCAGCCGTACGCCA |
| | | | GAGGTGACATGTGTCGTGGTTGACGTGTCCCACGAAGATCCCGAA |
| | | | GTTAAGTTCAATTGGTATGTTGATGGTGTAGAGGTACACAATGCTA |
| | | | AGACTAAACCTCGCGAGGAGCAGTACGCCTCGACCTATCGTGTCG |
| | | | TGAGCGTTCTGACCGTCCTTCACCAAGATTGGCTTAACGGCAAAG |
| | | | AATATAAGTGCAAGGTAAGCAATAAAGCACTTCCGGCCCCAATCG |
| | | | AGAAAACCATTTCCAAGGCCAAAGGTCAACCAAGAGAACCCCAG |
| | | | GTGTATACTCTTCCGCCTTCTCGTGAGGAAATGACTAAAAATCAAG |
| | | | TATCCCTTACGTGTCTGGTTAAAGGTTTTTATCCTAGCGATATTGCT |
| | | | GTTGAATGGGAATCGAACGGTCAGCCGGAGAATAATTATAAAACA |
| | | | ACGCCACCCGTCCTGGATAGCGACGGCTCATTTTTTCTGTATAGCA |
| | | | AACTGACTGTAGATAAATCACGGTGGCAGCAGGGCAATGTATTCA |
| | | | GTTGCTCCGTTATGCATGAAGCGTTACATAATCACTACACGCAGAA |
| | | | ATCTCTTAGTCTTTCACCCGGT |
| 431 | ATX-P-607 | HC IgG1 Fc- | CAGGTGCAGCTGGTGCAATCTGGGGCTGAGGTGAAGAAGCCTGGG |
| | | nucleotide | GCCTCAGTGAAGGTTTCCTGCAAGGCATCTGGATACACCTTCACCA |
| | | | GTTACTATATGCACTGGGTGCGACAGGCCCCTGGACAAGGGCTTG |
| | | | AGTGGATGGGAATAATCAACCCTAGTGGTGGTAGCACAAGCTACG |
| | | | CACAGAAGTTCCAGGGCAGAGTCACCATGACCAGGGACACGTCAA |
| | | | CGAACACAGTCTACATGGAGCTGAGCAGCCTTCGATCTGAGGACA |
| | | | CGGCCGTGTATTACTGTGCGAGAGATCCCGAGTATAGCAGTGCTTT |
| | | | TGATATCTGGGGACAAGGGACAATGGTCACCGTCTCCTCAGCTAG |
| | | | CACTAAAGGGCCTTCTGTATTTCCCTTGGCCCCGTCCAGCAAATCG |
| | | | ACCTCGGGAGGGACAGCCGCCCTGGGTTGCCTTGTGAAAGATTAT |
| | | | TTCCCTGAGCCAGTTACCGTAAGTTGGAACAGTGGGGCGCTGACA |
| | | | AGTGGTGTGCACACGTTTCCTGCCGTCCTGCAATCATCGGGCTTGT |
| | | | ATAGCCTCAGCTCTGTGGTCACTGTCCCAAGTTCATCGCTGGGCAC |
| | | | TCAGACGTATATTTGCAATGTGAACCACAAACCTTCAAATACAAA |
| | | | AGTGGATAAACGCGTAGAACCGAAATCGTGTGATAAAACTCACAC |
| | | | ATGCCCGCCATGCCCGGCACCTGAACTGCTTGGTGGTCCCAGCGTG |
| | | | TTCCTGTTCCCGCCGAAGCCTAAAGATACTCTAATGATCAGCCGTA |
| | | | CGCCAGAGGTGACATGTGTCGTGGTTGACGTGTCCCACGAAGATC |
| | | | CCGAAGTTAAGTTCAATTGGTATGTTGATGGTGTAGAGGTACACA |
| | | | ATGCTAAGACTAAACCTCGCGAGGAGCAGTACGCCTCGACCTATC |
| | | | GTGTCGTGAGCGTTCTGACCGTCCTTCACCAAGATTGGCTTAACGG |
| | | | CAAAGAATATAAGTGCAAGGTAAGCAATAAAGCACTTCCGGCCCC |
| | | | AATCGAGAAAACCATTTCCAAGGCCAAAGGTCAACCAAGAGAACC |
| | | | CCAGGTGTATACTCTTCCGCCTTCTCGTGAGGAAATGACTAAAAAT |
| | | | CAAGTATCCCTTACGTGTCTGGTTAAAGGTTTTTATCCTAGCGATA |
| | | | TTGCTGTTGAATGGGAATCGAACGGTCAGCCGGAGAATAATTATA |
| | | | AAACAACGCCACCCGTCCTGGATAGCGACGGCTCATTTTTTCTGTA |
| | | | TAGCAAACTGACTGTAGATAAATCACGGTGGCAGCAGGGCAATGT |
| | | | ATTCAGTTGCTCCGTTATGCATGAAGCGTTACATAATCACTACACG |
| | | | CAGAAATCTCTTAGTCTTTCACCCGGT |
| 432 | ATX-P-583 | LC Kappa | DIQMTQSPSSLSASVGDRVTITCRASQNIGSYLNWYQQKPGNAPKLLI |
| | | | YATSTLQSGVPSRFSGSGSGTDFTLIISSLHPEDFTVYYCQQSFIIPFTFG |
| | | | PGTKVDIKRTVAAPSVFIFPPSDEQLKSGTASVVCLLNNFYPREAKVQ |
| | | | WKVDNALQSGNSQESVTEQDSKDSTYSLSSTLTLSKADYEKHKVYA |
| | | | CEVTHQGLSSPVTKSFNRGEC |
| 433 | ATX-P-591 | LC Kappa | DIQMTQSPSSLSASVGDRVTITCRASQSINTYLNWYQQKPGKAPKLLI |
| | | | YAASSLQSGVPSRFSGSGSGTDFTLTISSLQPEDFATYSCQHSFTTPFTF |
| | | | GPGTKLEIKRTVAAPSVFIFPPSDEQLKSGTASVVCLLNNFYPREAKV |
| | | | QWKVDNALQSGNSQESVTEQDSKDSTYSLSSTLTLSKADYEKHKVY |
| | | | ACEVTHQGLSSPVTKSFNRGEC |
| 434 | ATX-P-602 | LC Kappa | EIVLTQSPGTLSLSPGERATLSCRASQSVSSSYLAWYQQKPGQAPRLLI |
| | | | YGASSRATGIPDRFSGSGSGTQFTLTISSLQSEDFAVYYCQQYGSSPWT |
| | | | FGQGTKVDIKRTVAAPSVFIFPPSDEQLKSGTASVVCLLNNFYPREAK |
| | | | VQWKVDNALQSGNSQESVTEQDSKDSTYSLSSTLTLSKADYEKHKV |
| | | | YACEVTHQGLSSPVTKSFNRGEC |
| 435 | ATX-P-597 | LC Kappa | DIQMTQSPSSLSASIGDRVTITCRASQSISSYLDWYQRKSGKAPKLLIY |
| | | | AASSLQSGVPSRFSGSGSGTDFTLTISSLQPEDFATYYCQQSYSTPLTF |
| | | | GGGTKVDIKRTVAAPSVFIFPPSDEQLKSGTASVVCLLNNFYPREAKV |
| | | | QWKVDNALQSGNSQESVTEQDSKDSTYSLSSTLTLSKADYEKHKVY |
| | | | ACEVTHQGLSSPVTKSFNRGEC |
| 436 | ATX-P-601 | LC Kappa | DIQMTQSPSSLSASVGDRVTITCRASQSINIYLNWYQQKPEKDPKLLIY |
| | | | AASSLQSGVPSRFSGSGSGTDFTLTISNLQPEDFATYYCQQSYSTPITFG |
| | | | QGTRLEIKRTVAAPSVFIFPPSDEQLKSGTASVVCLLNNFYPREAKVQ |
| | | | WKVDNALQSGNSQESVTEQDSKDSTYSLSSTLTLSKADYEKHKVYA |
| | | | CEVTHQGLSSPVTKSFNRGEC |
| 437 | ATX-P-607 | LC Kappa | DIQMTQSPSSLSASVGDRGTITCRASQSIRSYLNWYQQKPGKAPKLLI |
| | | | YAASSLQSGVPSRFSGSGSGTDFTLTISSLQPEDFATYYCQQSYRTPVT |
| | | | FGPGTKVDIKRTVAAPSVFIFPPSDEQLKSGTASVVCLLNNFYPREAK |
| | | | VQWKVDNALQSGNSQESVTEQDSKDSTYSLSSTLTLSKADYEKHKV |
| | | | YACEVTHQGLSSPVTKSFNRGEC |
| 438 | ATX-P-583 | LC Kappa- | GACATCCAGATGACCCAGTCTCCGTCCTCCCTGTCTGCATCTGTAG |
| | | nucleotide | GAGACAGAGTCACCATCACTTGCCGGGCAAGTCAGAACATTGGCA |
| | | | GTTATTTAAATTGGTATCAGCAGAAACCAGGGAATGCCCCTAAAC |
| | | | TCCTGATCTATGCTACATCCACTTTGCAAAGTGGGGTCCCATCAAG |
| | | | GTTCAGTGGCAGTGGATCTGGGACAGATTTCACTCTCATCATCAGC |
| | | | AGTCTGCACCCTGAAGATTTTACAGTTTATTACTGTCAACAGAGTT |
| | | | TCATTATCCCATTCACTTTCGGCCCTGGGACCAAAGTGGATATCAA |
| | | | ACGTACGGTAGCTGCCCCTTCAGTTTTTATCTTTCCGCCGTCTGAC |
| | | | GAGCAGTTAAAATCCGGGACCGCTTCTGTAGTTTGCCTGCTGAATA |
| | | | ATTTTTATCCGCGTGAGGCTAAAGTACAATGGAAAGTCGACAATG |
| | | | CTTTGCAGTCGGGAAATTCACAGGAAAGTGTTACGGAGCAGGATT |
| | | | CTAAAGATTCCACATATTCACTCAGCTCCACCCTTACACTGAGCAA |
| | | | AGCCGACTATGAAAAACATAAAGTTTACGCATGTGAGGTGACGCA |
| | | | CCAAGGATTATCCAGTCCGGTCACAAAATCGTTTAACCGCGGTGA |
| | | | GTGT |
| 439 | ATX-P-591 | LC Kappa- | GACATCCAGATGACTCAGTCTCCATCCTCCCTGTCTGCATCTGTTG |
| | | nucleotide | GAGACAGAGTCACCATCACTTGCCGGGCAAGTCAGAGCATTAACA |
| | | | CCTATTTAAATTGGTATCAGCAGAAACCAGGGAAAGCCCCTAAGC |
| | | | TCCTGATCTATGCTGCATCCAGTTTGCAAAGTGGGGTCCCATCAAG |
| | | | GTTCAGTGGCAGTGGATCTGGGACAGATTTCACTCTCACCATCAGC |
| | | | AGTCTGCAACCTGAAGATTTTGCAACTTACTCCTGTCAACATAGTT |
| | | | TCACTACCCCATTCACTTTCGGCCCTGGGACCAAGCTGGAAATCAA |
| | | | ACGTACGGTAGCTGCCCCTTCAGTTTTTATCTTTCCGCCGTCTGAC |
| | | | GAGCAGTTAAAATCCGGGACCGCTTCTGTAGTTTGCCTGCTGAATA |
| | | | ATTTTTATCCGCGTGAGGCTAAAGTACAATGGAAAGTCGACAATG |
| | | | CTTTGCAGTCGGGAAATTCACAGGAAAGTGTTACGGAGCAGGATT |
| | | | CTAAAGATTCCACATATTCACTCAGCTCCACCCTTACACTGAGCAA |
| | | | AGCCGACTATGAAAAACATAAAGTTTACGCATGTGAGGTGACGCA |
| | | | CCAAGGATTATCCAGTCCGGTCACAAAATCGTTTAACCGCGGTGA |
| | | | GTGT |
| 440 | ATX-P-602 | LC Kappa- | GAAATTGTGTTGACGCAGTCTCCAGGCACCCTGTCTTTGTCTCCAG |
| | | nucleotide | GGGAAAGAGCCACCCTCTCCTGCAGGGCCAGTCAGAGTGTTAGCA |
| | | | GCAGCTACTTAGCCTGGTACCAGCAGAAACCTGGCCAGGCTCCCA |
| | | | GGCTCCTCATCTATGGTGCATCCAGTAGGGCCACTGGCATCCCAGA |
| | | | CAGGTTCAGTGGCAGTGGGTCTGGGACACAGTTCACTCTCACCATC |
| | | | AGCAGCCTGCAGTCTGAAGATTTTGCAGTTTATTACTGTCAGCAGT |
| | | | ATGGTAGCTCACCGTGGACGTTCGGCCAAGGGACCAAAGTGGATA |
| | | | TCAAACGTACGGTAGCTGCCCCTTCAGTTTTTATCTTTCCGCCGTCT |
| | | | GACGAGCAGTTAAAATCCGGGACCGCTTCTGTAGTTTGCCTGCTGA |
| | | | ATAATTTTTATCCGCGTGAGGCTAAAGTACAATGGAAAGTCGACA |
| | | | ATGCTTTGCAGTCGGGAAATTCACAGGAAAGTGTTACGGAGCAGG |
| | | | ATTCTAAAGATTCCACATATTCACTCAGCTCCACCCTTACACTGAG |
| | | | CAAAGCCGACTATGAAAAACATAAAGTTTACGCATGTGAGGTGAC |
| | | | GCACCAAGGATTATCCAGTCCGGTCACAAAATCGTTTAACCGCGG |
| | | | TGAGTGT |
| 441 | ATX-P-597 | LC Kappa- | GACATCCAGATGACCCAGTCTCCATCCTCCCTGTCTGCATCTATTG |
| | | nucleotide | GAGACAGAGTCACCATCACTTGCCGGGCAAGTCAGAGCATTAGCA |
| | | | GCTATTTAGATTGGTATCAGCGGAAATCAGGGAAAGCCCCTAAAC |
| | | | TCCTGATCTATGCTGCATCCAGTTTGCAAAGTGGGGTCCCATCAAG |
| | | | GTTCAGTGGCAGTGGATCTGGGACAGATTTCACTCTCACCATCAGC |
| | | | AGTCTGCAACCTGAAGATTTTGCAACTTACTACTGTCAACAGAGTT |
| | | | ACAGTACCCCGCTCACTTTCGGCGGAGGGACCAAAGTGGATATCA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 442 | ATX-P-601 | LC Kappa- | GACATCCAGATGACCCAGTCTCCATCCTCCCTGTCTGCATCTGTAG |
| | | nucleotide | GAGACAGAGTCACCATCACTTGCCGGGCAAGTCAGAGCATTAATA |
| | | | TCTATTTAAATTGGTATCAGCAGAAACCAGAGAAAGACCCTAAGC |
| | | | TCCTGATCTATGCTGCGTCCAGTTTGCAAAGTGGGGTCCCATCAAG |
| | | | GTTCAGTGGCAGTGGATCTGGGACAGATTTCACTCTCACCATCAGC |
| | | | AATCTGCAACCTGAAGATTTTGCAACTTACTACTGTCAACAGAGTT |
| | | | ACAGTACCCCGATCACCTTCGGCCAAGGGACACGACTGGAGATTA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 443 | ATX-P-607 | LC Kappa- | GACATCCAGATGACACAGTCTCCATCCTCCCTGTCTGCATCTGTAG |
| | | nucleotide | GAGACAGAGGCACCATCACTTGCCGGGCAAGTCAGAGCATTAGAA |
| | | | GTTATTTAAATTGGTATCAGCAGAAACCAGGGAAAGCCCCTAAAC |
| | | | TCCTGATCTATGCTGCATCCAGTTTGCAAAGTGGGGTCCCATCAAG |
| | | | GTTCAGTGGCAGTGGATCTGGGACAGATTTCACTCTCACCATCAGC |
| | | | AGTCTGCAACCTGAAGATTTTGCAACTTACTACTGTCAACAGAGTT |
| | | | ACAGAACCCCAGTCACTTTCGGCCCTGGGACCAAAGTGGATATCA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 444 | ATX-P-564 | HC IgG1 Fc | EVQLVESGGGLVKPGGSLRLSCAASGFTFSSYTMNWVRQAPGKGLE |
| | | | WVSSISSSSSYTYYADSVRGRFTISRDNAKNSLYLQMSSLRAEDTAVY |
| | | | YCARSASIAAGMDVWGKGTTVTVSSASTKGPSVFPLAPSSKSTSGGT |
| | | | AALGCLVKDYFPEPVTVSWNSGALTSGVHTFPAVLQSSGLYSLSSVV |
| | | | TVPSSSLGTQTYICNVNHKPSNTKVDKRVEPKSCDKTHTCPPCPAPEL |
| | | | LGGPSVFLFPPKPKDTLMISRTPEVTCVVVDVSHEDPEVKFNWYVDG |
| | | | VEVHNAKTKPREEQYASTYRVVSVLTVLHQDWLNGKEYKCKVSNK |
| | | | ALPAPIEKTISKAKGQPREPQVYTLPPSREEMTKNQVSLTCLVKGFYPS |
| | | | DIAVEWESNGQPENNYKTTPPVLDSDGSFFLYSKLTVDKSRWQQGNV |
| | | | FSCSVMHEALHNHYTQKSLSLSPG |
| 445 | ATX-P-568 | HC IgG1 Fc | EVQLVESGGGLVQPGGSLRLSCAASGFPFSSYVMSWVRQAPGKGLE |
| | | | WVSVISVSGGSTFYADSVRGRFTISRDNSKNTLYLQVYSLRAEDTAV |
| | | | YYCAKDGLAVAGFDHWGQGTLVTVSSASTKGPSVFPLAPSSKSTSGG |
| | | | TAALGCLVKDYFPEPVTVSWNSGALTSGVHTFPAVLQSSGLYSLSSV |
| | | | VTVPSSSLGTQTYICNVNHKPSNTKVDKRVEPKSCDKTHTCPPCPAPE |
| | | | LLGGPSVFLFPPKPKDTLMISRTPEVTCVVVDVSHEDPEVKFNWYVD |
| | | | GVEVHNAKTKPREEQYASTYRVVSVLTVLHQDWLNGKEYKCKVSN |
| | | | KALPAPIEKTISKAKGQPREPQVYTLPPSREEMTKNQVSLTCLVKGFY |
| | | | PSDIAVEWESNGQPENNYKTTPPVLDSDGSFFLYSKLTVDKSRWQQG |
| | | | NVFSCSVMHEALHNHYTQKSLSLSPG |
| 446 | ATX-P-574 | HC IgG1 Fc | EVQLVESGGGLVQRGGSLRLSCVASGFTFSSYAMSWVRQAPGKGLE |
| | | | WVSVISGSGGTTYYADSVKGRFTISRDDSKKTLYLQMNSLRAEDTAV |
| | | | YYCAKRGDYGDYMDVWGKGTTVTVSSASTKGPSVFPLAPSSKSTSG |
| | | | GTAALGCLVKDYFPEPVTVSWNSGALTSGVHTFPAVLQSSGLYSLSS |
| | | | VVTVPSSSLGTQTYICNVNHKPSNTKVDKRVEPKSCDKTHTCPPCPAP |
| | | | ELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 447 | ATX-P-565 | HC IgG1 Fc | QVQLVESGGGVVQPGRSLRLSCAASGFTFNTYGIHWVRQAPGKGLE |
| | | | WVAVIWHDGSSKNYVDSVTGRFTISRDNSKNTVYLQMNSLRAEDTA |
| | | | VYYCARRGNWNYGFYYYYMDVWGKGTTVTVSSASTKGPSVFPLAP |
| | | | SSKSTSGGTAALGCLVKDYFPEPVTVSWNSGALTSGVHTFPAVLQSS |
| | | | GLYSLSSVVTVPSSSLGTQTYICNVNHKPSNTKVDKRVEPKSCDKTHT |
| | | | CPPCPAPELLGGPSVFLFPPKPKDTLMISRTPEVTCVVVDVSHEDPEVK |
| | | | FNWYVDGVEVHNAKTKPREEQYASTYRVVSVLTVLHQDWLNGKEY |
| | | | KCKVSNKALPAPIEKTISKAKGQPREPQVYTLPPSREEMTKNQVSLTC |
| | | | LVKGFYPSDIAVEWESNGQPENNYKTTPPVLDSDGSFFLYSKLTVDKS |
| | | | RWQQGNVFSCSVMHEALHNHYTQKSLSLSPG |
| 448 | ATX-P-578 | HC IgG1 Fc | EVQLVESGGGLVQPGGSLRLSCAASGFTFSRYSMNWVRQAPGKGLE |
| | | | WVSYISSSSSTIFHADSVKGRFTISRDNAKNSLYLQMNSLRDEDTAVY |
| | | | YCARGNWNYYYMDVWGKGTTVTVSSASTKGPSVFPLAPSSKSTSGG |
| | | | TAALGCLVKDYFPEPVTVSWNSGALTSGVHTFPAVLQSSGLYSLSSV |
| | | | VTVPSSSLGTQTYICNVNHKPSNTKVDKRVEPKSCDKTHTCPPCPAPE |
| | | | LLGGPSVFLFPPKPKDTLMISRTPEVTCVVVDVSHEDPEVKFNWYVD |
| | | | GVEVHNAKTKPREEQYASTYRVVSVLTVLHQDWLNGKEYKCKVSN |
| | | | KALPAPIEKTISKAKGQPREPQVYTLPPSREEMTKNQVSLTCLVKGFY |
| | | | PSDIAVEWESNGQPENNYKTTPPVLDSDGSFFLYSKLTVDKSRWQQG |
| | | | NVFSCSVMHEALHNHYTQKSLSLSPG |
| 449 | ATX-P-569 | HC IgG1 Fc | EVQLVESGGGLAQFGRSLRLSCAASGFTFDDYAMHWLRQAPGMGLE |
| | | | WVSGISWNSGSIGYADSVKGRFTIARDNAKNSVHLQMNSLRAEDTAL |
| | | | YYCAKDIGNGYTKGDVFDIWGQGTMVTVSSASTKGPSVFPLAPSSKS |
| | | | TSGGTAALGCLVKDYFPEPVTVSWNSGALTSGVHTFPAVLQSSGLYS |
| | | | LSSVVTVPSSSLGTQTYICNVNHKPSNTKVDKRVEPKSCDKTHTCPPC |
| | | | PAPELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVYTLPPSREEMTKNQVSLTCLVK |
| | | | GFYPSDIAVEWESNGQPENNYKTTPPVLDSDGSFFLYSKLTVDKSRW |
| | | | QQGNVFSCSVMHEALHNHYTQKSLSLSPG |
| 450 | ATX-P-576 | HC IgG1 Fc | EVQLVESGGGLVQPGRSLRLSCTASGFTFDDYEMHWVRQAPGKGLE |
| | | | WVSDISRNSGRIGYADSVKGRFTISRDNAKNSLYLQMNSLRAEDTAL |
| | | | YYCAKDIWGSGYFDYWGQGILVTVSSASTKGPSVFPLAPSSKSTSGGT |
| | | | AALGCLVKDYFPEPVTVSWNSGALTSGVHTFPAVLQSSGLYSLSSVV |
| | | | TVPSSSLGTQTYICNVNHKPSNTKVDKRVEPKSCDKTHTCPPCPAPEL |
| | | | LGGPSVFLFPPKPKDTLMISRTPEVTCVVVDVSHEDPEVKFNWYVDG |
| | | | VEVHNAKTKPREEQYASTYRVVSVLTVLHQDWLNGKEYKCKVSNK |
| | | | ALPAPIEKTISKAKGQPREPQVYTLPPSREEMTKNQVSLTCLVKGFYPS |
| | | | DIAVEWESNGQPENNYKTTPPVLDSDGSFFLYSKLTVDKSRWQQGNV |
| | | | FSCSVMHEALHNHYTQKSLSLSPG |
| 451 | ATX-P-588 | HC IgG1 Fc | EVQLVESGGGLVQPGRSLRLSCAASGFTFEDYAMHWVRQAPGKGLE |
| | | | WVSGISWNSGSIGYADSVKGRFTISRDNAKKSLYLQMNSLRAEDTAL |
| | | | YYCTKDRDDGYNYAGVFDYWGQGTLVTVSSASTKGPSVFPLAPSSK |
| | | | STSGGTAALGCLVKDYFPEPVTVSWNSGALTSGVHTFPAVLQSSGLY |
| | | | SLSSVVTVPSSSLGTQTYICNVNHKPSNTKVDKRVEPKSCDKTHTCPP |
| | | | CPAPELLGGPSVFLFPPKPKDTLMISRTPEVTCVVVDVSHEDPEVKFN |
| | | | WYVDGVEVHNAKTKPREEQYASTYRVVSVLTVLHQDWLNGKEYKC |
| | | | KVSNKALPAPIEKTISKAKGQPREPQVYTLPPSREEMTKNQVSLTCLV |
| | | | KGFYPSDIAVEWESNGQPENNYKTTPPVLDSDGSFFLYSKLTVDKSR |
| | | | WQQGNVFSCSVMHEALHNHYTQKSLSLSPG |
| 452 | ATX-P-596 | HC IgG1 Fc | EVQLVESGGGLVQPGRSLRLSCAASGFSFDDYAMHWVRQVPGKGLE |
| | | | WVSGISWTGGSVGYADPVKGRFTISRDNAKNSLYLQMNSLRPEDTAL |
| | | | YYCAKDMGDGYSRNWPFDYWGQGTLVTVSSASTKGPSVFPLAPSSK |
| | | | STSGGTAALGCLVKDYFPEPVTVSWNSGALTSGVHTFPAVLQSSGLY |
| | | | SLSSVVTVPSSSLGTQTYICNVNHKPSNTKVDKRVEPKSCDKTHTCPP |
| | | | CPAPELLGGPSVFLFPPKPKDTLMISRTPEVTCVVVDVSHEDPEVKFN |
| | | | WYVDGVEVHNAKTKPREEQYASTYRVVSVLTVLHQDWLNGKEYKC |
| | | | KVSNKALPAPIEKTISKAKGQPREPQVYTLPPSREEMTKNQVSLTCLV |
| | | | KGFYPSDIAVEWESNGQPENNYKTTPPVLDSDGSFFLYSKLTVDKSR |
| | | | WQQGNVFSCSVMHEALHNHYTQKSLSLSPG |
| 453 | ATX-P-600 | HC IgG1 Fc | EVQLVESGGGLVQPGRSLRLSCAASGFTFDDYAMHWVRQVPGRGLE |
| | | | WVSGISWSGGSIGYADSVKGRFTISRDNSKNSLYLQMNSLRAEDTAL |
| | | | YYCTKDREDGYNYGGVFDYWGQGTLVTVSSASTKGPSVFPLAPSSKS |
| | | | TSGGTAALGCLVKDYFPEPVTVSWNSGALTSGVHTFPAVLQSSGLYS |
| | | | LSSVVTVPSSSLGTQTYICNVNHKPSNTKVDKRVEPKSCDKTHTCPPC |
| | | | PAPELLGGPSVFLFPPKPKDTLMISRTPEVTCVVVDVSHEDPEVKFNW |
| | | | YVDGVEVHNAKTKPREEQYASTYRVVSVLTVLHQDWLNGKEYKCK |
| | | | VSNKALPAPIEKTISKAKGQPREPQVYTLPPSREEMTKNQVSLTCLVK |
| | | | GFYPSDIAVEWESNGQPENNYKTTPPVLDSDGSFFLYSKLTVDKSRW |
| | | | QQGNVFSCSVMHEALHNHYTQKSLSLSPG |
| 454 | ATX-P-564 | HC IgG1 Fc- | GAGGTGCAGCTGGTGGAGTCTGGGGGAGGCCTGGTCAAGCCTGGG |
| | | nucleotide | GGGTCCCTGAGACTCTCCTGTGCAGCCTCTGGATTCACCTTCAGTA |
| | | | GCTATACCATGAACTGGGTCCGCCAGGCTCCAGGGAAGGGGCTGG |
| | | | AGTGGGTGTCATCCATTAGTAGTAGTAGTAGTTACACATACTACGC |
| | | | AGACTCAGTGAGGGGCCGATTCACCATCTCCAGAGACAACGCCAA |
| | | | GAACTCACTGTATCTGCAAATGAGCAGCCTGAGAGCCGAGGACAC |
| | | | AGCTGTGTATTACTGTGCGAGATCGGCGAGTATAGCAGCTGGGAT |
| | | | GGACGTCTGGGGCAAAGGGACCACGGTCACCGTCTCCTCAGCTAG |
| | | | CACTAAAGGGCCTTCTGTATTTCCCTTGGCCCCGTCCAGCAAATCG |
| | | | ACCTCGGGAGGGACAGCCGCCCTGGGTTGCCTTGTGAAAGATTAT |
| | | | TTCCCTGAGCCAGTTACCGTAAGTTGGAACAGTGGGGCGCTGACA |
| | | | AGTGGTGTGCACACGTTTCCTGCCGTCCTGCAATCATCGGGCTTGT |
| | | | ATAGCCTCAGCTCTGTGGTCACTGTCCCAAGTTCATCGCTGGGCAC |
| | | | TCAGACGTATATTTGCAATGTGAACCACAAACCTTCAAATACAAA |
| | | | AGTGGATAAACGCGTAGAACCGAAATCGTGTGATAAAACTCACAC |
| | | | ATGCCCGCCATGCCCGGCACCTGAACTGCTTGGTGGTCCCAGCGTG |
| | | | TTCCTGTTCCCGCCGAAGCCTAAAGATACTCTAATGATCAGCCGTA |
| | | | CGCCAGAGGTGACATGTGTCGTGGTTGACGTGTCCCACGAAGATC |
| | | | CCGAAGTTAAGTTCAATTGGTATGTTGATGGTGTAGAGGTACACA |
| | | | ATGCTAAGACTAAACCTCGCGAGGAGCAGTACGCCTCGACCTATC |
| | | | GTGTCGTGAGCGTTCTGACCGTCCTTCACCAAGATTGGCTTAACGG |
| | | | CAAAGAATATAAGTGCAAGGTAAGCAATAAAGCACTTCCGGCCCC |
| | | | AATCGAGAAAACCATTTCCAAGGCCAAAGGTCAACCAAGAGAACC |
| | | | CCAGGTGTATACTCTTCCGCCTTCTCGTGAGGAAATGACTAAAAAT |
| | | | CAAGTATCCCTTACGTGTCTGGTTAAAGGTTTTTATCCTAGCGATA |
| | | | TTGCTGTTGAATGGGAATCGAACGGTCAGCCGGAGAATAATTATA |
| | | | AAACAACGCCACCCGTCCTGGATAGCGACGGCTCATTTTTTCTGTA |
| | | | TAGCAAACTGACTGTAGATAAATCACGGTGGCAGCAGGGCAATGT |
| | | | ATTCAGTTGCTCCGTTATGCATGAAGCGTTACATAATCACTACACG |
| | | | CAGAAATCTCTTAGTCTTTCACCCGGT |
| 455 | ATX-P-568 | HC IgG1 Fc- | GAGGTGCAGCTGGTGGAGTCTGGGGGAGGCTTGGTACAGCCGGGG |
| | | nucleotide | GGGTCCCTGAGACTCTCCTGTGCAGCCTCTGGATTCCCCTTTAGCA |
| | | | GCTATGTCATGAGCTGGGTCCGCCAGGCTCCAGGGAAGGGGCTGG |
| | | | AGTGGGTCTCAGTTATAAGTGTTAGTGGTGGTAGTACATTCTACGC |
| | | | AGACTCCGTGAGGGGCCGGTTCACCATCTCCAGAGACAATTCCAA |
| | | | GAACACGCTGTATCTGCAAGTGTACAGCCTGCGAGCCGAGGACAC |
| | | | GGCCGTATATTACTGTGCGAAAGATGGTTTGGCAGTGGCTGGTTTT |
| | | | GACCACTGGGGCCAGGGAACCCTGGTCACCGTCTCCTCAGCTAGC |
| | | | ACTAAAGGGCCTTCTGTATTTCCCTTGGCCCCGTCCAGCAAATCGA |
| | | | CCTCGGGAGGGACAGCCGCCCTGGGTTGCCTTGTGAAAGATTATTT |
| | | | CCCTGAGCCAGTTACCGTAAGTTGGAACAGTGGGGCGCTGACAAG |
| | | | TGGTGTGCACACGTTTCCTGCCGTCCTGCAATCATCGGGCTTGTAT |
| | | | AGCCTCAGCTCTGTGGTCACTGTCCCAAGTTCATCGCTGGGCACTC |
| | | | AGACGTATATTTGCAATGTGAACCACAAACCTTCAAATACAAAAG |
| | | | TGGATAAACGCGTAGAACCGAAATCGTGTGATAAAACTCACACAT |
| | | | GCCCGCCATGCCCGGCACCTGAACTGCTTGGTGGTCCCAGCGTGTT |
| | | | CCTGTTCCCGCCGAAGCCTAAAGATACTCTAATGATCAGCCGTACG |
| | | | CCAGAGGTGACATGTGTCGTGGTTGACGTGTCCCACGAAGATCCC |
| | | | GAAGTTAAGTTCAATTGGTATGTTGATGGTGTAGAGGTACACAAT |
| | | | GCTAAGACTAAACCTCGCGAGGAGCAGTACGCCTCGACCTATCGT |
| | | | GTCGTGAGCGTTCTGACCGTCCTTCACCAAGATTGGCTTAACGGCA |
| | | | AAGAATATAAGTGCAAGGTAAGCAATAAAGCACTTCCGGCCCCAA |
| | | | TCGAGAAAACCATTTCCAAGGCCAAAGGTCAACCAAGAGAACCCC |
| | | | AGGTGTATACTCTTCCGCCTTCTCGTGAGGAAATGACTAAAAATCA |
| | | | AGTATCCCTTACGTGTCTGGTTAAAGGTTTTTATCCTAGCGATATT |
| | | | GCTGTTGAATGGGAATCGAACGGTCAGCCGGAGAATAATTATAAA |
| | | | ACAACGCCACCCGTCCTGGATAGCGACGGCTCATTTTTTCTGTATA |
| | | | GCAAACTGACTGTAGATAAATCACGGTGGCAGCAGGGCAATGTAT |
| | | | TCAGTTGCTCCGTTATGCATGAAGCGTTACATAATCACTACACGCA |
| | | | GAAATCTCTTAGTCTTTCACCCGGT |
| 456 | ATX-P-574 | HC IgG1 Fc- | GAGGTGCAGCTGGTGGAGTCTGGGGGAGGCTTGGTACAGCGGGGG |
| | | nucleotide | GGGTCCCTGAGACTCTCCTGTGTAGCCTCTGGATTCACCTTTAGTA |
| | | | GCTATGCCATGAGCTGGGTCCGCCAGGCTCCAGGGAAGGGGCTGG |
| | | | AGTGGGTCTCAGTTATTAGTGGTAGTGGTGGTACCACATACTACGC |
| | | | AGACTCCGTGAAGGGCCGGTTCACCATCTCCAGAGACGATTCCAA |
| | | | GAAGACACTGTATCTGCAAATGAACAGCCTGAGAGCCGAGGACAC |
| | | | GGCCGTATATTATTGTGCGAAAAGGGGTGACTACGGGGACTACAT |
| | | | GGACGTCTGGGGCAAAGGGACCACGGTCACCGTCTCCTCAGCTAG |
| | | | CACTAAAGGGCCTTCTGTATTTCCCTTGGCCCCGTCCAGCAAATCG |
| | | | ACCTCGGGAGGGACAGCCGCCCTGGGTTGCCTTGTGAAAGATTAT |
| | | | TTCCCTGAGCCAGTTACCGTAAGTTGGAACAGTGGGGCGCTGACA |
| | | | AGTGGTGTGCACACGTTTCCTGCCGTCCTGCAATCATCGGGCTTGT |
| | | | ATAGCCTCAGCTCTGTGGTCACTGTCCCAAGTTCATCGCTGGGCAC |
| | | | TCAGACGTATATTTGCAATGTGAACCACAAACCTTCAAATACAAA |
| | | | AGTGGATAAACGCGTAGAACCGAAATCGTGTGATAAAACTCACAC |
| | | | ATGCCCGCCATGCCCGGCACCTGAACTGCTTGGTGGTCCCAGCGTG |
| | | | TTCCTGTTCCCGCCGAAGCCTAAAGATACTCTAATGATCAGCCGTA |
| | | | CGCCAGAGGTGACATGTGTCGTGGTTGACGTGTCCCACGAAGATC |
| | | | CCGAAGTTAAGTTCAATTGGTATGTTGATGGTGTAGAGGTACACA |
| | | | ATGCTAAGACTAAACCTCGCGAGGAGCAGTACGCCTCGACCTATC |
| | | | GTGTCGTGAGCGTTCTGACCGTCCTTCACCAAGATTGGCTTAACGG |
| | | | CAAAGAATATAAGTGCAAGGTAAGCAATAAAGCACTTCCGGCCCC |
| | | | AATCGAGAAAACCATTTCCAAGGCCAAAGGTCAACCAAGAGAACC |
| | | | CCAGGTGTATACTCTTCCGCCTTCTCGTGAGGAAATGACTAAAAAT |
| | | | CAAGTATCCCTTACGTGTCTGGTTAAAGGTTTTTATCCTAGCGATA |
| | | | TTGCTGTTGAATGGGAATCGAACGGTCAGCCGGAGAATAATTATA |
| | | | AAACAACGCCACCCGTCCTGGATAGCGACGGCTCATTTTTTCTGTA |
| | | | TAGCAAACTGACTGTAGATAAATCACGGTGGCAGCAGGGCAATGT |
| | | | ATTCAGTTGCTCCGTTATGCATGAAGCGTTACATAATCACTACACG |
| | | | CAGAAATCTCTTAGTCTTTCACCCGGT |
| 457 | ATX-P-565 | HC IgG1 Fc- | CAGGTGCAGCTGGTGGAGTCTGGGGGAGGCGTGGTCCAGCCTGGG |
| | | nucleotide | AGGTCCCTGAGACTCTCCTGTGCAGCGTCTGGATTCACCTTCAATA |
| | | | CCTATGGCATCCACTGGGTCCGCCAGGCTCCAGGCAAGGGACTGG |
| | | | AGTGGGTGGCAGTTATATGGCATGATGGAAGTAGTAAAAACTATG |
| | | | TAGACTCCGTGACGGGCCGATTCACCATCTCCAGAGACAATTCCA |
| | | | AGAACACGGTGTATCTGCAAATGAACAGCCTGAGAGCCGAGGACA |
| | | | CGGCTGTTTATTACTGTGCGAGAAGGGGTAACTGGAACTACGGCT |
| | | | TCTACTACTACTACATGGACGTCTGGGGCAAAGGAACCACGGTCA |
| | | | CCGTCTCCTCAGCTAGCACTAAAGGGCCTTCTGTATTTCCCTTGGC |
| | | | CCCGTCCAGCAAATCGACCTCGGGAGGGACAGCCGCCCTGGGTTG |
| | | | CCTTGTGAAAGATTATTTCCCTGAGCCAGTTACCGTAAGTTGGAAC |
| | | | AGTGGGGCGCTGACAAGTGGTGTGCACACGTTTCCTGCCGTCCTGC |
| | | | AATCATCGGGCTTGTATAGCCTCAGCTCTGTGGTCACTGTCCCAAG |
| | | | TTCATCGCTGGGCACTCAGACGTATATTTGCAATGTGAACCACAAA |
| | | | CCTTCAAATACAAAAGTGGATAAACGCGTAGAACCGAAATCGTGT |
| | | | GATAAAACTCACACATGCCCGCCATGCCCGGCACCTGAACTGCTT |
| | | | GGTGGTCCCAGCGTGTTCCTGTTCCCGCCGAAGCCTAAAGATACTC |
| | | | TAATGATCAGCCGTACGCCAGAGGTGACATGTGTCGTGGTTGACG |
| | | | TGTCCCACGAAGATCCCGAAGTTAAGTTCAATTGGTATGTTGATGG |
| | | | TGTAGAGGTACACAATGCTAAGACTAAACCTCGCGAGGAGCAGTA |
| | | | CGCCTCGACCTATCGTGTCGTGAGCGTTCTGACCGTCCTTCACCAA |
| | | | GATTGGCTTAACGGCAAAGAATATAAGTGCAAGGTAAGCAATAAA |
| | | | GCACTTCCGGCCCCAATCGAGAAAACCATTTCCAAGGCCAAAGGT |
| | | | CAACCAAGAGAACCCCAGGTGTATACTCTTCCGCCTTCTCGTGAGG |
| | | | AAATGACTAAAAATCAAGTATCCCTTACGTGTCTGGTTAAAGGTTT |
| | | | TTATCCTAGCGATATTGCTGTTGAATGGGAATCGAACGGTCAGCCG |
| | | | GAGAATAATTATAAAACAACGCCACCCGTCCTGGATAGCGACGGC |
| | | | TCATTTTTTCTGTATAGCAAACTGACTGTAGATAAATCACGGTGGC |
| | | | AGCAGGGCAATGTATTCAGTTGCTCCGTTATGCATGAAGCGTTACA |
| | | | TAATCACTACACGCAGAAATCTCTTAGTCTTTCACCCGGT |
| 458 | ATX-P-578 | HC IgG1 Fc- | GAGGTGCAGCTGGTGGAGTCTGGGGGAGGTTTGGTACAGCCTGGG |
| | | nucleotide | GGGTCCCTGAGACTCTCCTGTGCAGCCTCTGGATTCACCTTCAGTA |
| | | | GATATAGCATGAACTGGGTCCGCCAGGCTCCAGGGAAGGGGCTGG |
| | | | AGTGGGTTTCATACATTAGTAGTAGTAGTAGTACCATTTTCCATGC |
| | | | AGACTCTGTGAAGGGCCGATTCACCATCTCCAGAGACAATGCCAA |
| | | | GAACTCACTGTACCTGCAAATGAACAGCCTGAGAGACGAGGACAC |
| | | | GGCTGTGTATTACTGTGCGAGAGGGAACTGGAACTACTACTACAT |
| | | | GGACGTCTGGGGCAAAGGGACCACGGTCACCGTCTCCTCAGCTAG |
| | | | CACTAAAGGGCCTTCTGTATTTCCCTTGGCCCCGTCCAGCAAATCG |
| | | | ACCTCGGGAGGGACAGCCGCCCTGGGTTGCCTTGTGAAAGATTAT |
| | | | TTCCCTGAGCCAGTTACCGTAAGTTGGAACAGTGGGGCGCTGACA |
| | | | AGTGGTGTGCACACGTTTCCTGCCGTCCTGCAATCATCGGGCTTGT |
| | | | ATAGCCTCAGCTCTGTGGTCACTGTCCCAAGTTCATCGCTGGGCAC |
| | | | TCAGACGTATATTTGCAATGTGAACCACAAACCTTCAAATACAAA |
| | | | AGTGGATAAACGCGTAGAACCGAAATCGTGTGATAAAACTCACAC |
| | | | ATGCCCGCCATGCCCGGCACCTGAACTGCTTGGTGGTCCCAGCGTG |
| | | | TTCCTGTTCCCGCCGAAGCCTAAAGATACTCTAATGATCAGCCGTA |
| | | | CGCCAGAGGTGACATGTGTCGTGGTTGACGTGTCCCACGAAGATC |
| | | | CCGAAGTTAAGTTCAATTGGTATGTTGATGGTGTAGAGGTACACA |
| | | | ATGCTAAGACTAAACCTCGCGAGGAGCAGTACGCCTCGACCTATC |
| | | | GTGTCGTGAGCGTTCTGACCGTCCTTCACCAAGATTGGCTTAACGG |
| | | | CAAAGAATATAAGTGCAAGGTAAGCAATAAAGCACTTCCGGCCCC |
| | | | AATCGAGAAAACCATTTCCAAGGCCAAAGGTCAACCAAGAGAACC |
| | | | CCAGGTGTATACTCTTCCGCCTTCTCGTGAGGAAATGACTAAAAAT |
| | | | CAAGTATCCCTTACGTGTCTGGTTAAAGGTTTTTATCCTAGCGATA |
| | | | TTGCTGTTGAATGGGAATCGAACGGTCAGCCGGAGAATAATTATA |
| | | | AAACAACGCCACCCGTCCTGGATAGCGACGGCTCATTTTTTCTGTA |
| | | | TAGCAAACTGACTGTAGATAAATCACGGTGGCAGCAGGGCAATGT |
| | | | ATTCAGTTGCTCCGTTATGCATGAAGCGTTACATAATCACTACACG |
| | | | CAGAAATCTCTTAGTCTTTCACCCGGT |
| 459 | ATX-P-569 | HC IgG1 Fc- | GAAGTGCAGCTGGTGGAGTCTGGGGGAGGCTTGGCACAGTTTGGC |
| | | nucleotide | AGGTCCCTGAGACTCTCCTGTGCAGCCTCTGGATTCACCTTTGATG |
| | | | ATTATGCCATGCACTGGCTCCGGCAAGCTCCAGGGATGGGCCTGG |
| | | | AGTGGGTCTCAGGTATTAGTTGGAATAGCGGGAGCATAGGCTATG |
| | | | CGGACTCTGTGAAGGGCCGATTCACCATCGCCAGAGACAACGCCA |
| | | | AGAACTCCGTGCATCTGCAAATGAACAGTCTGAGAGCTGAGGACA |
| | | | CGGCCTTGTATTACTGTGCAAAAGATATAGGAAATGGATATACGA |
| | | | AGGGTGATGTTTTTGATATCTGGGGACAAGGGACAATGGTCACCG |
| | | | TCTCTTCAGCTAGCACTAAAGGGCCTTCTGTATTTCCCTTGGCCCC |
| | | | GTCCAGCAAATCGACCTCGGGAGGGACAGCCGCCCTGGGTTGCCT |
| | | | TGTGAAAGATTATTTCCCTGAGCCAGTTACCGTAAGTTGGAACAGT |
| | | | GGGGCGCTGACAAGTGGTGTGCACACGTTTCCTGCCGTCCTGCAAT |
| | | | CATCGGGCTTGTATAGCCTCAGCTCTGTGGTCACTGTCCCAAGTTC |
| | | | ATCGCTGGGCACTCAGACGTATATTTGCAATGTGAACCACAAACC |
| | | | TTCAAATACAAAAGTGGATAAACGCGTAGAACCGAAATCGTGTGA |
| | | | TAAAACTCACACATGCCCGCCATGCCCGGCACCTGAACTGCTTGGT |
| | | | GGTCCCAGCGTGTTCCTGTTCCCGCCGAAGCCTAAAGATACTCTAA |
| | | | TGATCAGCCGTACGCCAGAGGTGACATGTGTCGTGGTTGACGTGT |
| | | | CCCACGAAGATCCCGAAGTTAAGTTCAATTGGTATGTTGATGGTGT |
| | | | AGAGGTACACAATGCTAAGACTAAACCTCGCGAGGAGCAGTACGC |
| | | | CTCGACCTATCGTGTCGTGAGCGTTCTGACCGTCCTTCACCAAGAT |
| | | | TGGCTTAACGGCAAAGAATATAAGTGCAAGGTAAGCAATAAAGCA |
| | | | CTTCCGGCCCCAATCGAGAAAACCATTTCCAAGGCCAAAGGTCAA |
| | | | CCAAGAGAACCCCAGGTGTATACTCTTCCGCCTTCTCGTGAGGAA |
| | | | ATGACTAAAAATCAAGTATCCCTTACGTGTCTGGTTAAAGGTTTTT |
| | | | ATCCTAGCGATATTGCTGTTGAATGGGAATCGAACGGTCAGCCGG |
| | | | AGAATAATTATAAAACAACGCCACCCGTCCTGGATAGCGACGGCT |
| | | | CATTTTTTCTGTATAGCAAACTGACTGTAGATAAATCACGGTGGCA |
| | | | GCAGGGCAATGTATTCAGTTGCTCCGTTATGCATGAAGCGTTACAT |
| | | | AATCACTACACGCAGAAATCTCTTAGTCTTTCACCCGGT |
| 460 | ATX-P-576 | HC IgG1 Fc- | GAAGTGCAGCTGGTGGAGTCTGGGGGAGGCTTGGTACAGCCTGGC |
| | | nucleotide | AGGTCCCTGAGACTCTCCTGTACAGCCTCTGGATTCACATTTGATG |
| | | | ATTATGAAATGCACTGGGTCCGGCAAGCTCCAGGGAAGGGCCTGG |
| | | | AGTGGGTCTCAGATATTAGTCGGAACAGTGGTAGAATAGGCTATG |
| | | | CGGACTCTGTGAAGGGCCGATTCACCATTTCCAGAGACAACGCCA |
| | | | AGAACTCCCTGTATCTACAAATGAACAGTCTGAGAGCTGAGGACA |
| | | | CGGCCTTGTATTACTGTGCAAAAGATATTTGGGGATCGGGGTACTT |
| | | | TGACTATTGGGGCCAGGGAATCCTGGTCACCGTCTCCTCAGCTAGC |
| | | | ACTAAAGGGCCTTCTGTATTTCCCTTGGCCCCGTCCAGCAAATCGA |
| | | | CCTCGGGAGGGACAGCCGCCCTGGGTTGCCTTGTGAAAGATTATTT |
| | | | CCCTGAGCCAGTTACCGTAAGTTGGAACAGTGGGGCGCTGACAAG |
| | | | TGGTGTGCACACGTTTCCTGCCGTCCTGCAATCATCGGGCTTGTAT |
| | | | AGCCTCAGCTCTGTGGTCACTGTCCCAAGTTCATCGCTGGGCACTC |
| | | | AGACGTATATTTGCAATGTGAACCACAAACCTTCAAATACAAAAG |
| | | | TGGATAAACGCGTAGAACCGAAATCGTGTGATAAAACTCACACAT |
| | | | GCCCGCCATGCCCGGCACCTGAACTGCTTGGTGGTCCCAGCGTGTT |
| | | | CCTGTTCCCGCCGAAGCCTAAAGATACTCTAATGATCAGCCGTACG |
| | | | CCAGAGGTGACATGTGTCGTGGTTGACGTGTCCCACGAAGATCCC |
| | | | GAAGTTAAGTTCAATTGGTATGTTGATGGTGTAGAGGTACACAAT |
| | | | GCTAAGACTAAACCTCGCGAGGAGCAGTACGCCTCGACCTATCGT |
| | | | GTCGTGAGCGTTCTGACCGTCCTTCACCAAGATTGGCTTAACGGCA |
| | | | AAGAATATAAGTGCAAGGTAAGCAATAAAGCACTTCCGGCCCCAA |
| | | | TCGAGAAAACCATTTCCAAGGCCAAAGGTCAACCAAGAGAACCCC |
| | | | AGGTGTATACTCTTCCGCCTTCTCGTGAGGAAATGACTAAAAATCA |
| | | | AGTATCCCTTACGTGTCTGGTTAAAGGTTTTTATCCTAGCGATATT |
| | | | GCTGTTGAATGGGAATCGAACGGTCAGCCGGAGAATAATTATAAA |
| | | | ACAACGCCACCCGTCCTGGATAGCGACGGCTCATTTTTTCTGTATA |
| | | | GCAAACTGACTGTAGATAAATCACGGTGGCAGCAGGGCAATGTAT |
| | | | TCAGTTGCTCCGTTATGCATGAAGCGTTACATAATCACTACACGCA |
| | | | GAAATCTCTTAGTCTTTCACCCGGT |
| 461 | ATX-P-588 | HC IgG1 Fc- | GAAGTGCAGCTGGTGGAGTCCGGGGGAGGCTTGGTACAACCTGGC |
| | | nucleotide | AGGTCCCTGAGACTCTCCTGTGCAGCCTCTGGATTCACCTTTGAAG |
| | | | ATTATGCCATGCACTGGGTCCGGCAAGCTCCAGGGAAGGGCCTGG |
| | | | AGTGGGTCTCAGGTATTAGTTGGAATAGTGGTAGCATAGGCTATG |
| | | | CGGACTCTGTGAAGGGCCGATTCACCATCTCCAGAGACAACGCCA |
| | | | AGAAATCCCTGTATCTGCAAATGAACAGTCTGAGAGCTGAGGACA |
| | | | CGGCCTTATATTACTGTACAAAAGATAGAGATGATGGCTACAACT |
| | | | ATGCGGGGGTCTTTGACTACTGGGGCCAGGGAACCCTGGTCACTG |
| | | | TCTCCTCAGCTAGCACTAAAGGGCCTTCTGTATTTCCCTTGGCCCC |
| | | | GTCCAGCAAATCGACCTCGGGAGGGACAGCCGCCCTGGGTTGCCT |
| | | | TGTGAAAGATTATTTCCCTGAGCCAGTTACCGTAAGTTGGAACAGT |
| | | | GGGGCGCTGACAAGTGGTGTGCACACGTTTCCTGCCGTCCTGCAAT |
| | | | CATCGGGCTTGTATAGCCTCAGCTCTGTGGTCACTGTCCCAAGTTC |
| | | | ATCGCTGGGCACTCAGACGTATATTTGCAATGTGAACCACAAACC |
| | | | TTCAAATACAAAAGTGGATAAACGCGTAGAACCGAAATCGTGTGA |
| | | | TAAAACTCACACATGCCCGCCATGCCCGGCACCTGAACTGCTTGGT |
| | | | GGTCCCAGCGTGTTCCTGTTCCCGCCGAAGCCTAAAGATACTCTAA |
| | | | TGATCAGCCGTACGCCAGAGGTGACATGTGTCGTGGTTGACGTGT |
| | | | CCCACGAAGATCCCGAAGTTAAGTTCAATTGGTATGTTGATGGTGT |
| | | | AGAGGTACACAATGCTAAGACTAAACCTCGCGAGGAGCAGTACGC |
| | | | CTCGACCTATCGTGTCGTGAGCGTTCTGACCGTCCTTCACCAAGAT |
| | | | TGGCTTAACGGCAAAGAATATAAGTGCAAGGTAAGCAATAAAGCA |
| | | | CTTCCGGCCCCAATCGAGAAAACCATTTCCAAGGCCAAAGGTCAA |
| | | | CCAAGAGAACCCCAGGTGTATACTCTTCCGCCTTCTCGTGAGGAA |
| | | | ATGACTAAAAATCAAGTATCCCTTACGTGTCTGGTTAAAGGTTTTT |
| | | | ATCCTAGCGATATTGCTGTTGAATGGGAATCGAACGGTCAGCCGG |
| | | | AGAATAATTATAAAACAACGCCACCCGTCCTGGATAGCGACGGCT |
| | | | CATTTTTTCTGTATAGCAAACTGACTGTAGATAAATCACGGTGGCA |
| | | | GCAGGGCAATGTATTCAGTTGCTCCGTTATGCATGAAGCGTTACAT |
| | | | AATCACTACACGCAGAAATCTCTTAGTCTTTCACCCGGT |
| 462 | ATX-P-596 | HC IgG1 Fc- | GAAGTGCAGCTGGTGGAGTCTGGGGGAGGCTTGGTACAGCCTGGC |
| | | nucleotide | AGGTCCCTGAGACTCTCCTGTGCAGCCTCTGGGTTCAGTTTTGATG |
| | | | ATTATGCCATGCACTGGGTCCGGCAAGTTCCAGGGAAGGGCCTGG |
| | | | AATGGGTCTCAGGTATTAGTTGGACTGGTGGTAGCGTAGGCTATG |
| | | | CGGACCCCGTGAAGGGCCGATTCACCATCTCCAGAGACAACGCCA |
| | | | AGAACTCCCTGTATCTGCAAATGAACAGTCTGAGACCTGAGGACA |
| | | | CGGCCTTGTATTACTGTGCAAAAGATATGGGAGACGGGTATAGCA |
| | | | GAAACTGGCCCTTTGACTACTGGGGCCAGGGAACCCTGGTCACCG |
| | | | TCTCCTCAGCTAGCACTAAAGGGCCTTCTGTATTTCCCTTGGCCCC |
| | | | GTCCAGCAAATCGACCTCGGGAGGGACAGCCGCCCTGGGTTGCCT |
| | | | TGTGAAAGATTATTTCCCTGAGCCAGTTACCGTAAGTTGGAACAGT |
| | | | GGGGCGCTGACAAGTGGTGTGCACACGTTTCCTGCCGTCCTGCAAT |
| | | | CATCGGGCTTGTATAGCCTCAGCTCTGTGGTCACTGTCCCAAGTTC |
| | | | ATCGCTGGGCACTCAGACGTATATTTGCAATGTGAACCACAAACC |
| | | | TTCAAATACAAAAGTGGATAAACGCGTAGAACCGAAATCGTGTGA |
| | | | TAAAACTCACACATGCCCGCCATGCCCGGCACCTGAACTGCTTGGT |
| | | | GGTCCCAGCGTGTTCCTGTTCCCGCCGAAGCCTAAAGATACTCTAA |
| | | | TGATCAGCCGTACGCCAGAGGTGACATGTGTCGTGGTTGACGTGT |
| | | | CCCACGAAGATCCCGAAGTTAAGTTCAATTGGTATGTTGATGGTGT |
| | | | AGAGGTACACAATGCTAAGACTAAACCTCGCGAGGAGCAGTACGC |
| | | | CTCGACCTATCGTGTCGTGAGCGTTCTGACCGTCCTTCACCAAGAT |
| | | | TGGCTTAACGGCAAAGAATATAAGTGCAAGGTAAGCAATAAAGCA |
| | | | CTTCCGGCCCCAATCGAGAAAACCATTTCCAAGGCCAAAGGTCAA |
| | | | CCAAGAGAACCCCAGGTGTATACTCTTCCGCCTTCTCGTGAGGAA |
| | | | ATGACTAAAAATCAAGTATCCCTTACGTGTCTGGTTAAAGGTTTTT |
| | | | ATCCTAGCGATATTGCTGTTGAATGGGAATCGAACGGTCAGCCGG |
| | | | AGAATAATTATAAAACAACGCCACCCGTCCTGGATAGCGACGGCT |
| | | | CATTTTTTCTGTATAGCAAACTGACTGTAGATAAATCACGGTGGCA |
| | | | GCAGGGCAATGTATTCAGTTGCTCCGTTATGCATGAAGCGTTACAT |
| | | | AATCACTACACGCAGAAATCTCTTAGTCTTTCACCCGGT |
| 463 | ATX-P-600 | HC IgG1 Fc- | GAAGTGCAGCTGGTGGAGTCTGGGGGAGGCTTGGTACAGCCTGGC |
| | | nucleotide | AGGTCCCTGAGACTCTCCTGTGCAGCCTCTGGATTCACCTTTGATG |
| | | | ATTATGCCATGCACTGGGTCCGGCAAGTTCCAGGGAGGGGCCTGG |
| | | | AGTGGGTCTCAGGTATTAGTTGGAGTGGTGGCAGCATAGGCTATG |
| | | | CGGACTCTGTGAAGGGCCGATTCACCATCTCCAGAGACAACTCCA |
| | | | AGAACTCCCTGTATCTGCAAATGAACAGTCTGAGAGCTGAGGACA |
| | | | CGGCCTTGTATTACTGTACAAAAGATAGAGAAGATGGATACAACT |
| | | | ATGGGGGGGTCTTTGACTACTGGGGCCAGGGAACCCTGGTCACCG |
| | | | TCTCCTCAGCTAGCACTAAAGGGCCTTCTGTATTTCCCTTGGCCCC |
| | | | GTCCAGCAAATCGACCTCGGGAGGGACAGCCGCCCTGGGTTGCCT |
| | | | TGTGAAAGATTATTTCCCTGAGCCAGTTACCGTAAGTTGGAACAGT |
| | | | GGGGCGCTGACAAGTGGTGTGCACACGTTTCCTGCCGTCCTGCAAT |
| | | | CATCGGGCTTGTATAGCCTCAGCTCTGTGGTCACTGTCCCAAGTTC |
| | | | ATCGCTGGGCACTCAGACGTATATTTGCAATGTGAACCACAAACC |
| | | | TTCAAATACAAAAGTGGATAAACGCGTAGAACCGAAATCGTGTGA |
| | | | TAAAACTCACACATGCCCGCCATGCCCGGCACCTGAACTGCTTGGT |
| | | | GGTCCCAGCGTGTTCCTGTTCCCGCCGAAGCCTAAAGATACTCTAA |
| | | | TGATCAGCCGTACGCCAGAGGTGACATGTGTCGTGGTTGACGTGT |
| | | | CCCACGAAGATCCCGAAGTTAAGTTCAATTGGTATGTTGATGGTGT |
| | | | AGAGGTACACAATGCTAAGACTAAACCTCGCGAGGAGCAGTACGC |
| | | | CTCGACCTATCGTGTCGTGAGCGTTCTGACCGTCCTTCACCAAGAT |
| | | | TGGCTTAACGGCAAAGAATATAAGTGCAAGGTAAGCAATAAAGCA |
| | | | CTTCCGGCCCCAATCGAGAAAACCATTTCCAAGGCCAAAGGTCAA |
| | | | CCAAGAGAACCCCAGGTGTATACTCTTCCGCCTTCTCGTGAGGAA |
| | | | ATGACTAAAAATCAAGTATCCCTTACGTGTCTGGTTAAAGGTTTTT |
| | | | ATCCTAGCGATATTGCTGTTGAATGGGAATCGAACGGTCAGCCGG |
| | | | AGAATAATTATAAAACAACGCCACCCGTCCTGGATAGCGACGGCT |
| | | | CATTTTTTCTGTATAGCAAACTGACTGTAGATAAATCACGGTGGCA |
| | | | GCAGGGCAATGTATTCAGTTGCTCCGTTATGCATGAAGCGTTACAT |
| | | | AATCACTACACGCAGAAATCTCTTAGTCTTTCACCCGGT |
| 464 | ATX-P-564 | LC Kappa | DIVMTQSPLSLPVTPGEPASISCRSSQSLLHSTGYNFLDWYLQKPGLSP |
| | | | QLLIYLASNRASGVPDRFSGSGSGTDFTLKISRVEAEDVGVYYCMQA |
| | | | LQTPYTFGQGTKLEIKRTVAAPSVFIFPPSDEQLKSGTASVVCLLNNFY |
| | | | PREAKVQWKVDNALQSGNSQESVTEQDSKDSTYSLSSTLTLSKADYE |
| | | | KHKVYACEVTHQGLSSPVTKSFNRGEC |
| 465 | ATX-P-568 | LC Kappa | DIQMTQSPSTLSASVGDRVTITCRASQSISTWLAWYQQKPGKAPKLLI |
| | | | HKASGLESGVPSRFSGSGSGTEFTLTISSLQPDDFATYYCQQYNSFSLIF |
| | | | GGGTRVEIKRTVAAPSVFIFPPSDEQLKSGTASVVCLLNNFYPREAKV |
| | | | QWKVDNALQSGNSQESVTEQDSKDSTYSLSSTLTLSKADYEKHKVY |
| | | | ACEVTHQGLSSPVTKSFNRGEC |
| 466 | ATX-P-574 | LC Kappa | DIQMTQSPSSVSASVGDRVTITCRASQGITTWLAWYQQKPGKAPKLLI |
| | | | FGASSLESGVPSRFSGSGSGTDFTLTISSLQPEDFATYYCQQANSFPWT |
| | | | FGQGTKVEIKRTVAAPSVFIFPPSDEQLKSGTASVVCLLNNFYPREAK |
| | | | VQWKVDNALQSGNSQESVTEQDSKDSTYSLSSTLILSKADYEKHKV |
| | | | YACEVTHQGLSSPVTKSFNRGEC |
| 467 | ATX-P-565 | LC Kappa | DIVMTQSPLSLPVTPGEPASISCRSSQRLLHSSGYNYLDWYLQKPGQS |
| | | | PQLLIYLGSSRASGVPDRFSGSGSGTDFTLKISRVEAEDVGVYYCMQG |
| | | | LQTPPTFGQGTKVEIKRTVAAPSVFIFPPSDEQLKSGTASVVCLLNNFY |
| | | | PREAKVQWKVDNALQSGNSQESVTEQDSKDSTYSLSSTLTLSKADYE |
| | | | KHKVYACEVTHQGLSSPVTKSFNRGEC |
| 468 | ATX-P-578 | LC Kappa | DIQMTQSPSTLSASVGDRVTISCRASQSVNSWLAWYQQKPGKAPKLL |
| | | | IYKASNLEGGVPSSFSGSGSGTEFTLTISSLQPDDFATYYCQQYDTSWT |
| | | | FGQGTKVEIKRTVAAPSVFIFPPSDEQLKSGTASVVCLLNNFYPREAK |
| | | | VQWKVDNALQSGNSQESVTEQDSKDSTYSLSSTLTLSKADYEKHKV |
| | | | YACEVTHQGLSSPVTKSFNRGEC |
| 469 | ATX-P-569 | LC Kappa | DIQMTQSPSTLSASVGDSVTITCRASQNIITWLAWYQQKPGKAPNLLI |
| | | | YKASSLESGVPSRFSGSGSGTEFTLTISSLQPDDFATYYCQQYSSYSYT |
| | | | FGQGTKLEIKRTVAAPSVFIFPPSDEQLKSGTASVVCLLNNFYPREAK |
| | | | VQWKVDNALQSGNSQESVTEQDSKDSTYSLSSTLTLSKADYEKHKV |
| | | | YACEVTHQGLSSPVTKSFNRGEC |
| 470 | ATX-P-576 | LC Kappa | DIQMTQSPSSVSASVGDRVTITCRTSQGIRNWLAWYQQKPGKAPKLLI |
| | | | YAASSLQTGGPSRFSGSGSGTDFTLTISSLQPEDFATYYCQQANSFPYT |
| | | | FGQGTKLEIKRTVAAPSVFIFPPSDEQLKSGTASVVCLLNNFYPREAK |
| | | | VQWKVDNALQSGNSQESVTEQDSKDSTYSLSSTLTLSKADYEKHKV |
| | | | YACEVTHQGLSSPVTKSFNRGEC |
| 471 | ATX-P-588 | LC Kappa | EIVLTQSPATLSVSPGERATLSCRASQSVSSNLAWYQQKPGQAPRLLI |
| | | | YGASSRATGIPDRFSGSGSGTDFTLTISRLEPEDFAVYYCQQYGSSFTF |
| | | | GPGTKVDIKRTVAAPSVFIFPPSDEQLKSGTASVVCLLNNFYPREAKV |
| | | | QWKVDNALQSGNSQESVTEQDSKDSTYSLSSTLTLSKADYEKHKVY |
| | | | ACEVTHQGLSSPVTKSFNRGEC |
| 472 | ATX-P-596 | LC Kappa | DIVMTQSPDSLAVSLGERATINCKSSQSVLYSSNNKNYLAWYQQKPG |
| | | | QPPKLLIYWASTRESGVPDRFSGSGSGTDFTLTISSLQAEDVAVYYCQ |
| | | | QYGSSPMYTFGQGTKLEIKRTVAAPSVFIFPPSDEQLKSGTASVVCLL |
| | | | NNFYPREAKVQWKVDNALQSGNSQESVTEQDSKDSTYSLSSTLTLSK |
| | | | ADYEKHKVYACEVTHQGLSSPVTKSFNRGEC |
| 473 | ATX-P-600 | LC Kappa | EIVLTQSPGTLSLSPGERATLSCRASQNVRGSYLAWYQQKPGQAPRLL |
| | | | IYGASNRATDIPDRFSGSGSGTDFTLTISGLEPEDFVVYYCQQYGSSYT |
| | | | FGQGTKVEIKRTVAAPSVFIFPPSDEQLKSGTASVVCLLNNFYPREAK |
| | | | VQWKVDNALQSGNSQESVTEQDSKDSTYSLSSTLTLSKADYEKHKV |
| | | | YACEVTHQGLSSPVTKSFNRGEC |
| 474 | ATX-P-564 | LC Kappa- | GATATTGTGATGACTCAGTCTCCACTCTCCCTGCCCGTCACCCCTG |
| | | nucleotide | GAGAGCCGGCCTCCATCTCCTGCAGGTCTAGTCAGAGCCTCCTGCA |
| | | | TAGTACTGGATACAACTTTTTGGATTGGTACCTGCAGAAGCCAGG |
| | | | GCTGTCTCCACAGCTCCTGATCTATTTGGCTTCTAATCGGGCCTCC |
| | | | GGGGTCCCTGACAGGTTCAGTGGCAGTGGATCAGGCACAGATTTT |
| | | | ACACTGAAAATCAGCAGAGTGGAGGCTGAGGATGTTGGGGTTTAT |
| | | | TACTGCATGCAAGCTCTACAAACTCCGTACACTTTTGGCCAGGGGA |
| | | | CCAAGCTGGAGATCAAACGTACGGTAGCTGCCCCTTCAGTTTTTAT |
| | | | CTTTCCGCCGTCTGACGAGCAGTTAAAATCCGGGACCGCTTCTGTA |
| | | | GTTTGCCTGCTGAATAATTTTTATCCGCGTGAGGCTAAAGTACAAT |
| | | | GGAAAGTCGACAATGCTTTGCAGTCGGGAAATTCACAGGAAAGTG |
| | | | TTACGGAGCAGGATTCTAAAGATTCCACATATTCACTCAGCTCCAC |
| | | | CCTTACACTGAGCAAAGCCGACTATGAAAAACATAAAGTTTACGC |
| | | | ATGTGAGGTGACGCACCAAGGATTATCCAGTCCGGTCACAAAATC |
| | | | GTTTAACCGCGGTGAGTGT |
| 475 | ATX-P-568 | LC Kappa- | GACATCCAGATGACCCAGTCTCCTTCCACCCTGTCTGCCTCTGTAG |
| | | nucleotide | GAGACAGAGTCACCATCACTTGCCGGGCCAGCCAGAGTATTAGTA |
| | | | CCTGGTTGGCCTGGTATCAGCAGAAACCAGGGAAAGCCCCTAAGC |
| | | | TCCTGATCCATAAGGCGTCTGGTTTAGAAAGTGGGGTCCCATCAA |
| | | | GATTCAGCGGCAGTGGATCTGGGACAGAATTCACTCTCACCATCA |
| | | | GCAGCCTGCAGCCTGATGATTTTGCAACTTATTACTGCCAACAGTA |
| | | | TAATAGTTTTTCTCTCATTTTCGGCGGAGGGACCAGGGTGGAGATC |
| | | | AAACGTACGGTAGCTGCCCCTTCAGTTTTTATCTTTCCGCCGTCTG |
| | | | ACGAGCAGTTAAAATCCGGGACCGCTTCTGTAGTTTGCCTGCTGAA |
| | | | TAATTTTTATCCGCGTGAGGCTAAAGTACAATGGAAAGTCGACAA |
| | | | TGCTTTGCAGTCGGGAAATTCACAGGAAAGTGTTACGGAGCAGGA |
| | | | TTCTAAAGATTCCACATATTCACTCAGCTCCACCCTTACACTGAGC |
| | | | AAAGCCGACTATGAAAAACATAAAGTTTACGCATGTGAGGTGACG |
| | | | CACCAAGGATTATCCAGTCCGGTCACAAAATCGTTTAACCGCGGT |
| | | | GAGTGT |
| 476 | ATX-P-574 | LC Kappa- | GACATCCAGATGACCCAGTCTCCATCTTCCGTGTCTGCATCTGTAG |
| | | nucleotide | GAGACAGAGTCACCATCACTTGTCGGGCGAGTCAGGGTATTACCA |
| | | | CCTGGTTAGCCTGGTATCAGCAGAAACCAGGGAAAGCCCCTAAGC |
| | | | TCCTGATCTTTGGTGCATCTAGTTTGGAAAGTGGGGTCCCATCAAG |
| | | | GTTCAGCGGCAGTGGATCTGGGACAGATTTCACTCTCACCATCAGC |
| | | | AGCCTGCAGCCTGAAGATTTTGCAACTTACTATTGTCAACAGGCTA |
| | | | ACAGTTTCCCGTGGACGTTCGGCCAAGGGACCAAGGTGGAAATCA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 477 | ATX-P-565 | LC Kappa- | GATATTGTGATGACTCAGTCTCCACTCTCCCTGCCCGTCACCCCTG |
| | | nucleotide | GAGAGCCGGCCTCCATCTCCTGCAGGTCTAGTCAGCGCCTCCTGCA |
| | | | TAGTAGTGGATACAATTATTTGGATTGGTATCTGCAGAAGCCAGG |
| | | | ACAGTCTCCACAGCTCCTGATCTATTTGGGTTCTAGTCGGGCCTCC |
| | | | GGGGTCCCTGACAGGTTCAGTGGCAGTGGATCAGGCACAGATTTT |
| | | | ACACTGAAAATCAGCAGGGTGGAGGCTGAGGATGTTGGGGTTTAT |
| | | | TACTGCATGCAAGGTCTACAAACTCCTCCGACGTTCGGCCAAGGG |
| | | | ACCAAGGTGGAAATCAAACGTACGGTAGCTGCCCCTTCAGTTTTTA |
| | | | TCTTTCCGCCGTCTGACGAGCAGTTAAAATCCGGGACCGCTTCTGT |
| | | | AGTTTGCCTGCTGAATAATTTTTATCCGCGTGAGGCTAAAGTACAA |
| | | | TGGAAAGTCGACAATGCTTTGCAGTCGGGAAATTCACAGGAAAGT |
| | | | GTTACGGAGCAGGATTCTAAAGATTCCACATATTCACTCAGCTCCA |
| | | | CCCTTACACTGAGCAAAGCCGACTATGAAAAACATAAAGTTTACG |
| | | | CATGTGAGGTGACGCACCAAGGATTATCCAGTCCGGTCACAAAAT |
| | | | CGTTTAACCGCGGTGAGTGT |
| 478 | ATX-P-578 | LC Kappa- | GACATCCAGATGACCCAGTCTCCTTCCACCCTGTCTGCATCTGTAG |
| | | nucleotide | GAGACAGAGTCACCATCAGTTGCCGGGCCAGTCAGAGTGTTAATA |
| | | | GCTGGTTGGCCTGGTATCAGCAGAAACCAGGGAAAGCCCCTAAAC |
| | | | TCCTGATCTATAAGGCGTCTAATTTAGAAGGTGGGGTCCCATCAAG |
| | | | TTTCAGCGGCAGTGGATCTGGGACAGAATTCACTCTCACCATCAGC |
| | | | AGCCTGCAGCCTGATGATTTTGCAACTTATTACTGCCAACAATATG |
| | | | ATACTTCTTGGACGTTCGGCCAAGGGACCAAGGTGGAGATCAAAC |
| | | | GTACGGTAGCTGCCCCTTCAGTTTTTATCTTTCCGCCGTCTGACGA |
| | | | GCAGTTAAAATCCGGGACCGCTTCTGTAGTTTGCCTGCTGAATAAT |
| | | | TTTTATCCGCGTGAGGCTAAAGTACAATGGAAAGTCGACAATGCT |
| | | | TTGCAGTCGGGAAATTCACAGGAAAGTGTTACGGAGCAGGATTCT |
| | | | AAAGATTCCACATATTCACTCAGCTCCACCCTTACACTGAGCAAAG |
| | | | CCGACTATGAAAAACATAAAGTTTACGCATGTGAGGTGACGCACC |
| | | | AAGGATTATCCAGTCCGGTCACAAAATCGTTTAACCGCGGTGAGT |
| | | | GT |
| 479 | ATX-P-569 | LC Kappa- | GACATCCAGATGACCCAGTCTCCTTCCACCCTGTCTGCATCTGTAG |
| | | nucleotide | GAGACAGCGTCACCATCACTTGCCGGGCCAGTCAGAATATTATTA |
| | | | CCTGGTTGGCTTGGTATCAGCAGAAACCAGGGAAAGCCCCTAACC |
| | | | TCCTGATCTATAAGGCGTCTAGTTTAGAAAGTGGGGTCCCATCAAG |
| | | | GTTCAGCGGCAGTGGATCTGGGACAGAATTCACTCTCACCATCAG |
| | | | CAGCCTGCAGCCTGATGATTTTGCAACTTATTACTGCCAACAGTAT |
| | | | AGTAGTTATTCGTACACTTTTGGCCAGGGGACCAAGCTGGAGATC |
| | | | AAACGTACGGTAGCTGCCCCTTCAGTTTTTATCTTTCCGCCGTCTG |
| | | | ACGAGCAGTTAAAATCCGGGACCGCTTCTGTAGTTTGCCTGCTGAA |
| | | | TAATTTTTATCCGCGTGAGGCTAAAGTACAATGGAAAGTCGACAA |
| | | | TGCTTTGCAGTCGGGAAATTCACAGGAAAGTGTTACGGAGCAGGA |
| | | | TTCTAAAGATTCCACATATTCACTCAGCTCCACCCTTACACTGAGC |
| | | | AAAGCCGACTATGAAAAACATAAAGTTTACGCATGTGAGGTGACG |
| | | | CACCAAGGATTATCCAGTCCGGTCACAAAATCGTTTAACCGCGGT |
| | | | GAGTGT |
| 480 | ATX-P-576 | LC Kappa- | GACATCCAGATGACCCAGTCTCCATCTTCCGTGTCTGCATCTGTAG |
| | | nucleotide | GAGACAGAGTCACCATCACTTGTCGGACGAGTCAGGGTATTAGAA |
| | | | ACTGGTTAGCCTGGTATCAGCAGAAACCAGGGAAAGCCCCTAAAC |
| | | | TCCTGATCTATGCTGCATCCAGTTTGCAAACTGGGGGCCCATCAAG |
| | | | GTTCAGCGGCAGTGGATCTGGGACAGATTTCACTCTCACCATCAGC |
| | | | AGCCTGCAGCCTGAAGATTTTGCAACTTACTATTGTCAACAGGCTA |
| | | | ACAGTTTCCCGTACACTTTTGGCCAGGGGACCAAGCTGGAGATCA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 481 | ATX-P-588 | LC Kappa- | GAAATTGTGTTGACGCAGTCTCCAGCCACCCTGTCTGTGTCTCCAG |
| | | nucleotide | GGGAAAGAGCCACCCTCTCCTGCAGGGCCAGTCAGAGTGTTAGCA |
| | | | GCAACTTAGCCTGGTACCAGCAGAAACCTGGCCAGGCTCCCAGGC |
| | | | TCCTCATCTATGGTGCATCCAGCAGGGCCACTGGCATCCCAGACA |
| | | | GGTTCAGTGGCAGTGGGTCTGGGACAGACTTCACTCTCACCATCA |
| | | | GCAGACTGGAGCCTGAAGATTTTGCAGTGTATTACTGTCAGCAGT |
| | | | ATGGTAGCTCATTCACTTTCGGCCCTGGGACCAAAGTGGATATCAA |
| | | | ACGTACGGTAGCTGCCCCTTCAGTTTTTATCTTTCCGCCGTCTGAC |
| | | | GAGCAGTTAAAATCCGGGACCGCTTCTGTAGTTTGCCTGCTGAATA |
| | | | ATTTTTATCCGCGTGAGGCTAAAGTACAATGGAAAGTCGACAATG |
| | | | CTTTGCAGTCGGGAAATTCACAGGAAAGTGTTACGGAGCAGGATT |
| | | | CTAAAGATTCCACATATTCACTCAGCTCCACCCTTACACTGAGCAA |
| | | | AGCCGACTATGAAAAACATAAAGTTTACGCATGTGAGGTGACGCA |
| | | | CCAAGGATTATCCAGTCCGGTCACAAAATCGTTTAACCGCGGTGA |
| | | | GTGT |
| 482 | ATX-P-596 | LC Kappa- | GACATCGTGATGACTCAGTCTCCAGACTCCCTGGCTGTGTCTCTGG |
| | | nucleotide | GCGAGAGGGCCACCATCAACTGCAAGTCCAGCCAGAGTGTTTTAT |
| | | | ACAGCTCCAACAATAAGAACTACTTAGCTTGGTACCAGCAGAAAC |
| | | | CAGGACAGCCTCCTAAGCTGCTCATTTACTGGGCATCTACCCGGGA |
| | | | ATCCGGGGTCCCTGACCGATTCAGTGGCAGCGGGTCTGGGACAGA |
| | | | TTTCACTCTCACCATCAGCAGCCTGCAGGCTGAAGATGTGGCAGTT |
| | | | TATTACTGTCAGCAGTATGGTAGCTCACCCATGTACACTTTTGGCC |
| | | | AGGGGACCAAGCTGGAGATCAAACGTACGGTAGCTGCCCCTTCAG |
| | | | TTTTTATCTTTCCGCCGTCTGACGAGCAGTTAAAATCCGGGACCGC |
| | | | TTCTGTAGTTTGCCTGCTGAATAATTTTTATCCGCGTGAGGCTAAA |
| | | | GTACAATGGAAAGTCGACAATGCTTTGCAGTCGGGAAATTCACAG |
| | | | GAAAGTGTTACGGAGCAGGATTCTAAAGATTCCACATATTCACTC |
| | | | AGCTCCACCCTTACACTGAGCAAAGCCGACTATGAAAAACATAAA |
| | | | GTTTACGCATGTGAGGTGACGCACCAAGGATTATCCAGTCCGGTC |
| | | | ACAAAATCGTTTAACCGCGGTGAGTGT |
| 483 | ATX-P-600 | LC Kappa- | GAAATTGTGTTGACGCAGTCTCCAGGCACCCTGTCTTTGTCTCCAG |
| | | nucleotide | GGGAAAGAGCCACCCTCTCCTGCAGGGCCAGTCAGAATGTTAGGG |
| | | | GCAGCTACTTAGCCTGGTACCAGCAGAAACCTGGCCAGGCTCCCA |
| | | | GGCTCCTCATATATGGTGCATCCAACAGGGCCACTGACATCCCAG |
| | | | ACAGGTTCAGTGGCAGTGGGTCTGGGACAGACTTCACTCTCACCA |
| | | | TCAGCGGCCTGGAGCCTGAAGATTTTGTAGTGTATTACTGTCAGCA |
| | | | GTATGGTAGTTCGTACACTTTTGGCCAGGGGACCAAGGTGGAGAT |
| | | | CAAACGTACGGTAGCTGCCCCTTCAGTTTTTATCTTTCCGCCGTCT |
| | | | GACGAGCAGTTAAAATCCGGGACCGCTTCTGTAGTTTGCCTGCTGA |
| | | | ATAATTTTTATCCGCGTGAGGCTAAAGTACAATGGAAAGTCGACA |
| | | | ATGCTTTGCAGTCGGGAAATTCACAGGAAAGTGTTACGGAGCAGG |
| | | | ATTCTAAAGATTCCACATATTCACTCAGCTCCACCCTTACACTGAG |
| | | | CAAAGCCGACTATGAAAAACATAAAGTTTACGCATGTGAGGTGAC |
| | | | GCACCAAGGATTATCCAGTCCGGTCACAAAATCGTTTAACCGCGG |
| | | | TGAGTGT |
| 484 | ATX-P-571 | HC IgG1 Fc | QVQLQESGPGLVKPSGTLSLTCAVSGGSISSRNWWSWVRQPPGKGLE |
| | | | WIGEIYHGGTTNYNPSLKSRVTISVDKSKNQFSLKLSSVTAADTAVYY |
| | | | CARGGLRLGELSPNAFDIWGQGTMVTVSSASTKGPSVFPLAPSSKSTS |
| | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCPA |
| | | | PELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 485 | ATX-P-577 | HC IgG1 Fc | QVQLQESGPGLVKPSGTLSLTCAVSGGSISSSNWWTWVRQPPGKGLE |
| | | | WIGETYHDGSTNYNPSLKSRVTISVDKSKNQFSLKLSSVTAADTAVY |
| | | | YCLEFYFDYWGQGTLVTVSSASTKGPSVFPLAPSSKSTSGGTAALGCL |
| | | | VKDYFPEPVTVSWNSGALTSGVHTFPAVLQSSGLYSLSSVVTVPSSSL |
| | | | GTQTYICNVNHKPSNTKVDKRVEPKSCDKTHTCPPCPAPELLGGPSVF |
| | | | LFPPKPKDTLMISRTPEVTCVVVDVSHEDPEVKFNWYVDGVEVHNA |
| | | | KTKPREEQYASTYRVVSVLTVLHQDWLNGKEYKCKVSNKALPAPIE |
| | | | KTISKAKGQPREPQVYTLPPSREEMTKNQVSLTCLVKGFYPSDIAVEW |
| | | | ESNGQPENNYKTTPPVLDSDGSFFLYSKLTVDKSRWQQGNVFSCSVM |
| | | | HEALHNHYTQKSLSLSPG |
| 486 | ATX-P-579 | HC IgG1 Fc | QVQLQESGPGLVKPSGTLSLTCAVSGASISTSNWWTWVRQPPGKGLE |
| | | | WIGEIYHDGSTNYSPSLQSRVTISVDKSKNQFSLTLTSVTAADTAIYYC |
| | | | GDFYIDVWGNGTTVTVSSASTKGPSVFPLAPSSKSTSGGTAALGCLVK |
| | | | DYFPEPVTVSWNSGALTSGVHTFPAVLQSSGLYSLSSVVTVPSSSLGT |
| | | | QTYICNVNHKPSNTKVDKRVEPKSCDKTHTCPPCPAPELLGGPSVFLF |
| | | | PPKPKDTLMISRTPEVTCVVVDVSHEDPEVKFNWYVDGVEVHNAKT |
| | | | KPREEQYASTYRVVSVLTVLHQDWLNGKEYKCKVSNKALPAPIEKTI |
| | | | SKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMH |
| | | | EALHNHYTQKSLSLSPG |
| 487 | ATX-P-580 | HC IgG1 Fc | QVQLQESGPGLVKPSGTLSLTCAVSGGSISTSDWWTWVRQPPGKGLE |
| | | | WIGEIYHSGSTNYNPSLKSRVTISVDKSKNQFSLKLSSVTAADTAVYY |
| | | | CADWYFDLWGRGTLVTVSSASTKGPSVFPLAPSSKSTSGGTAALGCL |
| | | | VKDYFPEPVTVSWNSGALTSGVHTFPAVLQSSGLYSLSSVVTVPSSSL |
| | | | GTQTYICNVNHKPSNTKVDKRVEPKSCDKTHTCPPCPAPELLGGPSVF |
| | | | LFPPKPKDTLMISRTPEVTCVVVDVSHEDPEVKFNWYVDGVEVHNA |
| | | | KTKPREEQYASTYRVVSVLTVLHQDWLNGKEYKCKVSNKALPAPIE |
| | | | KTISKAKGQPREPQVYTLPPSREEMTKNQVSLTCLVKGFYPSDIAVEW |
| | | | ESNGQPENNYKTTPPVLDSDGSFFLYSKLTVDKSRWQQGNVFSCSVM |
| | | | HEALHNHYTQKSLSLSPG |
| 488 | ATX-P-582 | HC IgG1 Fc | QVQLQESGPGLVKPSGTLSLTCAVSGGSISISKWWSWVRQPPGKGLE |
| | | | WIGEIYHTGSTNYHPSLKSRVTISVDKSKNQFYLKLNSVTAADTAVY |
| | | | YCARDELRLGELSPSLDIWGQGTMVTVSSASTKGPSVFPLAPSSKSTS |
| | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCPA |
| | | | PELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 489 | ATX-P-598 | HC IgG1 Fc | QVQLQESGPGLVKPSGTLSLTCAVSGGSISSSNWWSWVRQSPGKGLE |
| | | | WIGEIYHSGTTNYNPSLKSRVTISVDKSKNQFSLKLSSATAADTAVYY |
| | | | CARDRLRLGESSSDVFDIWGQGTMVTVSSASTKGPSVFPLAPSSKSTS |
| | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCPA |
| | | | PELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 490 | ATX-P-603 | HC IgG1 Fc | QVQLQESGPGLVKPSGTLSLTCAVSGGSISGSHWWSWFRQPPGKGLE |
| | | | WIGEIYHTGSTNKNPSLKSRVTISLDKSKNQFSLRLRSVTDADTAVYY |
| | | | CARDQYTGSPRDAFDIWGQGTMVTVSSASTKGPSVFPLAPSSKSTSG |
| | | | GTAALGCLVKDYFPEPVTVSWNSGALTSGVHTFPAVLQSSGLYSLSS |
| | | | VVTVPSSSLGTQTYICNVNHKPSNTKVDKRVEPKSCDKTHTCPPCPAP |
| | | | ELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 491 | ATX-P-608 | HC IgG1 Fc | QVQLQESGPGLVKPSGTLSLTCAVSGGSISSSNWWSWVRQSPGKGLE |
| | | | WIGEIYHTGSTNKKPSLKSRVIISVDKSKNQFSLKLISVNAADTAIYYC |
| | | | ARDREGGTTEAFDIWGQGTMVTVSSASTKGPSVFPLAPSSKSTSGGTA |
| | | | ALGCLVKDYFPEPVTVSWNSGALTSGVHTFPAVLQSSGLYSLSSVVT |
| | | | VPSSSLGTQTYICNVNHKPSNTKVDKRVEPKSCDKTHTCPPCPAPELL |
| | | | GGPSVFLFPPKPKDTLMISRTPEVTCVVVDVSHEDPEVKFNWYVDGV |
| | | | EVHNAKTKPREEQYASTYRVVSVLTVLHQDWLNGKEYKCKVSNKA |
| | | | LPAPIEKTISKAKGQPREPQVYTLPPSREEMTKNQVSLTCLVKGFYPSD |
| | | | IAVEWESNGQPENNYKTTPPVLDSDGSFFLYSKLTVDKSRWQQGNVF |
| | | | SCSVMHEALHNHYTQKSLSLSPG |
| 492 | ATX-P-609 | HC IgG1 Fc | QVQLQESGPGLVKPSGTLSLTCAVSGGSISSSNWWSWVRQPPGKGLE |
| | | | WIGEIYHSGSTNYNPSLKSRVTISVDKSKNQFSLKLRSVTAADTAVYY |
| | | | CARDQLSSGPTDAFDIWGQGTMVTVSSASTKGPSVFPLAPSSKSTSGG |
| | | | TAALGCLVKDYFPEPVTVSWNSGALTSGVHTFPAVLQSSGLYSLSSV |
| | | | VTVPSSSLGTQTYICNVNHKPSNTKVDKRVEPKSCDKTHTCPPCPAPE |
| | | | LLGGPSVFLFPPKPKDTLMISRTPEVTCVVVDVSHEDPEVKFNWYVD |
| | | | GVEVHNAKTKPREEQYASTYRVVSVLTVLHQDWLNGKEYKCKVSN |
| | | | KALPAPIEKTISKAKGQPREPQVYTLPPSREEMTKNQVSLTCLVKGFY |
| | | | PSDIAVEWESNGQPENNYKTTPPVLDSDGSFFLYSKLTVDKSRWQQG |
| | | | NVFSCSVMHEALHNHYTQKSLSLSPG |
| 493 | ATX-P-563 | HC IgG1 Fc | QVQLQESGPGLMKPSETLSLTCTVSGGSISSHYWSWIRQPPGKGLEWI |
| | | | GYIFYSGNINYNPSLKSRVTISVDTSKNQFSLKLRSVTTADTAVYYCA |
| | | | RDRGGSYSYHFDCWGQGTLVTVSSASTKGPSVFPLAPSSKSTSGGTA |
| | | | ALGCLVKDYFPEPVTVSWNSGALTSGVHTFPAVLQSSGLYSLSSVVT |
| | | | VPSSSLGTQTYICNVNHKPSNTKVDKRVEPKSCDKTHTCPPCPAPELL |
| | | | GGPSVFLFPPKPKDTLMISRTPEVTCVVVDVSHEDPEVKFNWYVDGV |
| | | | EVHNAKTKPREEQYASTYRVVSVLTVLHQDWLNGKEYKCKVSNKA |
| | | | LPAPIEKTISKAKGQPREPQVYTLPPSREEMTKNQVSLTCLVKGFYPSD |
| | | | IAVEWESNGQPENNYKTTPPVLDSDGSFFLYSKLTVDKSRWQQGNVF |
| | | | SCSVMHEALHNHYTQKSLSLSPG |
| 494 | ATX-P-570 | HC IgG1 Fc | QVQLQESGPGLVKPSETMSLTCTVSGGSFTGYYWSWIRQPPGKGLEW |
| | | | IGYVHYSGGTKSNPSLKSRVTISVDTSKNQFSLKLSSVTAADTAVYYC |
| | | | ARERYEWNSYFDYWGQGTLVTVSSASTKGPSVFPLAPSSKSTSGGTA |
| | | | ALGCLVKDYFPEPVTVSWNSGALTSGVHTFPAVLQSSGLYSLSSVVT |
| | | | VPSSSLGTQTYICNVNHKPSNTKVDKRVEPKSCDKTHTCPPCPAPELL |
| | | | GGPSVFLFPPKPKDTLMISRTPEVTCVVVDVSHEDPEVKFNWYVDGV |
| | | | EVHNAKTKPREEQYASTYRVVSVLTVLHQDWLNGKEYKCKVSNKA |
| | | | LPAPIEKTISKAKGQPREPQVYTLPPSREEMTKNQVSLTCLVKGFYPSD |
| | | | IAVEWESNGQPENNYKTTPPVLDSDGSFFLYSKLTVDKSRWQQGNVF |
| | | | SCSVMHEALHNHYTQKSLSLSPG |
| 495 | ATX-P-571 | HC IgG1 Fc- | CAGGTGCAGCTGCAGGAGTCGGGCCCAGGACTGGTGAAGCCTTCG |
| | | nucleotide | GGGACCCTGTCCCTCACCTGTGCTGTCTCGGGTGGCTCCATCAGTA |
| | | | GTCGTAACTGGTGGAGTTGGGTCCGCCAGCCCCCAGGGAAGGGGC |
| | | | TGGAGTGGATTGGGGAAATCTATCATGGTGGGACCACCAACTACA |
| | | | ACCCGTCCCTTAAGAGTCGAGTCACCATATCAGTAGACAAGTCCA |
| | | | AGAACCAATTCTCCCTGAAGCTGAGTTCTGTGACCGCCGCGGACA |
| | | | CGGCCGTGTATTACTGTGCGAGAGGGGGATTACGTTTGGGGGAGT |
| | | | TATCGCCTAATGCTTTTGATATCTGGGGACAAGGGACAATGGTCAC |
| | | | CGTCTCTTCAGCTAGCACTAAAGGGCCTTCTGTATTTCCCTTGGCC |
| | | | CCGTCCAGCAAATCGACCTCGGGAGGGACAGCCGCCCTGGGTTGC |
| | | | CTTGTGAAAGATTATTTCCCTGAGCCAGTTACCGTAAGTTGGAACA |
| | | | GTGGGGCGCTGACAAGTGGTGTGCACACGTTTCCTGCCGTCCTGCA |
| | | | ATCATCGGGCTTGTATAGCCTCAGCTCTGTGGTCACTGTCCCAAGT |
| | | | TCATCGCTGGGCACTCAGACGTATATTTGCAATGTGAACCACAAA |
| | | | CCTTCAAATACAAAAGTGGATAAACGCGTAGAACCGAAATCGTGT |
| | | | GATAAAACTCACACATGCCCGCCATGCCCGGCACCTGAACTGCTT |
| | | | GGTGGTCCCAGCGTGTTCCTGTTCCCGCCGAAGCCTAAAGATACTC |
| | | | TAATGATCAGCCGTACGCCAGAGGTGACATGTGTCGTGGTTGACG |
| | | | TGTCCCACGAAGATCCCGAAGTTAAGTTCAATTGGTATGTTGATGG |
| | | | TGTAGAGGTACACAATGCTAAGACTAAACCTCGCGAGGAGCAGTA |
| | | | CGCCTCGACCTATCGTGTCGTGAGCGTTCTGACCGTCCTTCACCAA |
| | | | GATTGGCTTAACGGCAAAGAATATAAGTGCAAGGTAAGCAATAAA |
| | | | GCACTTCCGGCCCCAATCGAGAAAACCATTTCCAAGGCCAAAGGT |
| | | | CAACCAAGAGAACCCCAGGTGTATACTCTTCCGCCTTCTCGTGAGG |
| | | | AAATGACTAAAAATCAAGTATCCCTTACGTGTCTGGTTAAAGGTTT |
| | | | TTATCCTAGCGATATTGCTGTTGAATGGGAATCGAACGGTCAGCCG |
| | | | GAGAATAATTATAAAACAACGCCACCCGTCCTGGATAGCGACGGC |
| | | | TCATTTTTTCTGTATAGCAAACTGACTGTAGATAAATCACGGTGGC |
| | | | AGCAGGGCAATGTATTCAGTTGCTCCGTTATGCATGAAGCGTTACA |
| | | | TAATCACTACACGCAGAAATCTCTTAGTCTTTCACCCGGT |
| 496 | ATX-P-577 | HC IgG1 Fc- | CAGGTGCAGCTGCAGGAGTCGGGCCCAGGACTGGTGAAGCCTTCG |
| | | nucleotide | GGGACCCTGTCCCTCACCTGCGCTGTCTCTGGTGGCTCCATCAGCA |
| | | | GTAGTAACTGGTGGACTTGGGTCCGCCAGCCCCCAGGGAAGGGGC |
| | | | TGGAGTGGATTGGGGAAACCTATCATGATGGGAGCACCAACTACA |
| | | | ACCCGTCCCTCAAGAGTCGAGTCACCATTTCAGTAGACAAGTCCA |
| | | | AGAACCAATTCTCCCTGAAACTGAGTTCTGTGACCGCCGCGGACA |
| | | | CGGCCGTGTATTACTGTTTGGAATTCTACTTTGACTACTGGGGCCA |
| | | | GGGAACCCTGGTCACCGTCTCCTCAGCTAGCACTAAAGGGCCTTCT |
| | | | GTATTTCCCTTGGCCCCGTCCAGCAAATCGACCTCGGGAGGGACA |
| | | | GCCGCCCTGGGTTGCCTTGTGAAAGATTATTTCCCTGAGCCAGTTA |
| | | | CCGTAAGTTGGAACAGTGGGGCGCTGACAAGTGGTGTGCACACGT |
| | | | TTCCTGCCGTCCTGCAATCATCGGGCTTGTATAGCCTCAGCTCTGT |
| | | | GGTCACTGTCCCAAGTTCATCGCTGGGCACTCAGACGTATATTTGC |
| | | | AATGTGAACCACAAACCTTCAAATACAAAAGTGGATAAACGCGTA |
| | | | GAACCGAAATCGTGTGATAAAACTCACACATGCCCGCCATGCCCG |
| | | | GCACCTGAACTGCTTGGTGGTCCCAGCGTGTTCCTGTTCCCGCCGA |
| | | | AGCCTAAAGATACTCTAATGATCAGCCGTACGCCAGAGGTGACAT |
| | | | GTGTCGTGGTTGACGTGTCCCACGAAGATCCCGAAGTTAAGTTCA |
| | | | ATTGGTATGTTGATGGTGTAGAGGTACACAATGCTAAGACTAAAC |
| | | | CTCGCGAGGAGCAGTACGCCTCGACCTATCGTGTCGTGAGCGTTCT |
| | | | GACCGTCCTTCACCAAGATTGGCTTAACGGCAAAGAATATAAGTG |
| | | | CAAGGTAAGCAATAAAGCACTTCCGGCCCCAATCGAGAAAACCAT |
| | | | TTCCAAGGCCAAAGGTCAACCAAGAGAACCCCAGGTGTATACTCT |
| | | | TCCGCCTTCTCGTGAGGAAATGACTAAAAATCAAGTATCCCTTACG |
| | | | TGTCTGGTTAAAGGTTTTTATCCTAGCGATATTGCTGTTGAATGGG |
| | | | AATCGAACGGTCAGCCGGAGAATAATTATAAAACAACGCCACCCG |
| | | | TCCTGGATAGCGACGGCTCATTTTTTCTGTATAGCAAACTGACTGT |
| | | | AGATAAATCACGGTGGCAGCAGGGCAATGTATTCAGTTGCTCCGT |
| | | | TATGCATGAAGCGTTACATAATCACTACACGCAGAAATCTCTTAGT |
| | | | CTTTCACCCGGT |
| 497 | ATX-P-579 | HC IgG1 Fc- | CAGGTGCAGCTGCAGGAGTCGGGCCCAGGACTGGTGAAGCCTTCG |
| | | nucleotide | GGGACCCTGTCCCTCACCTGCGCTGTGTCTGGTGCCTCCATCAGCA |
| | | | CAAGTAATTGGTGGACTTGGGTCCGCCAGCCCCCAGGGAAGGGGC |
| | | | TGGAGTGGATTGGTGAAATCTATCATGATGGGAGCACCAACTACA |
| | | | GCCCGTCCCTCCAGAGTCGAGTCACCATATCAGTAGACAAGTCCA |
| | | | AGAACCAGTTCTCCCTGACGCTGACCTCTGTGACCGCCGCGGACA |
| | | | CGGCCATTTATTATTGTGGTGACTTCTACATAGACGTCTGGGGCAA |
| | | | CGGGACCACGGTCACCGTCTCCTCAGCTAGCACTAAAGGGCCTTCT |
| | | | GTATTTCCCTTGGCCCCGTCCAGCAAATCGACCTCGGGAGGGACA |
| | | | GCCGCCCTGGGTTGCCTTGTGAAAGATTATTTCCCTGAGCCAGTTA |
| | | | CCGTAAGTTGGAACAGTGGGGCGCTGACAAGTGGTGTGCACACGT |
| | | | TTCCTGCCGTCCTGCAATCATCGGGCTTGTATAGCCTCAGCTCTGT |
| | | | GGTCACTGTCCCAAGTTCATCGCTGGGCACTCAGACGTATATTTGC |
| | | | AATGTGAACCACAAACCTTCAAATACAAAAGTGGATAAACGCGTA |
| | | | GAACCGAAATCGTGTGATAAAACTCACACATGCCCGCCATGCCCG |
| | | | GCACCTGAACTGCTTGGTGGTCCCAGCGTGTTCCTGTTCCCGCCGA |
| | | | AGCCTAAAGATACTCTAATGATCAGCCGTACGCCAGAGGTGACAT |
| | | | GTGTCGTGGTTGACGTGTCCCACGAAGATCCCGAAGTTAAGTTCA |
| | | | ATTGGTATGTTGATGGTGTAGAGGTACACAATGCTAAGACTAAAC |
| | | | CTCGCGAGGAGCAGTACGCCTCGACCTATCGTGTCGTGAGCGTTCT |
| | | | GACCGTCCTTCACCAAGATTGGCTTAACGGCAAAGAATATAAGTG |
| | | | CAAGGTAAGCAATAAAGCACTTCCGGCCCCAATCGAGAAAACCAT |
| | | | TTCCAAGGCCAAAGGTCAACCAAGAGAACCCCAGGTGTATACTCT |
| | | | TCCGCCTTCTCGTGAGGAAATGACTAAAAATCAAGTATCCCTTACG |
| | | | TGTCTGGTTAAAGGTTTTTATCCTAGCGATATTGCTGTTGAATGGG |
| | | | AATCGAACGGTCAGCCGGAGAATAATTATAAAACAACGCCACCCG |
| | | | TCCTGGATAGCGACGGCTCATTTTTTCTGTATAGCAAACTGACTGT |
| | | | AGATAAATCACGGTGGCAGCAGGGCAATGTATTCAGTTGCTCCGT |
| | | | TATGCATGAAGCGTTACATAATCACTACACGCAGAAATCTCTTAGT |
| | | | CTTTCACCCGGT |
| 498 | ATX-P-580 | HC IgG1 Fc- | CAGGTGCAGCTGCAGGAGTCGGGCCCAGGACTGGTGAAGCCTTCG |
| | | nucleotide | GGGACCCTGTCCCTCACCTGCGCTGTCTCTGGTGGCTCCATCAGCA |
| | | | CTAGTGACTGGTGGACTTGGGTCCGCCAGCCCCCAGGGAAGGGGC |
| | | | TGGAGTGGATTGGGGAAATCTATCATAGTGGGAGCACCAACTACA |
| | | | ACCCGTCCCTCAAGAGTCGAGTCACCATATCAGTAGACAAATCCA |
| | | | AGAACCAGTTCTCCCTGAAGCTGAGCTCTGTGACCGCCGCGGACA |
| | | | CGGCCGTGTATTACTGTGCGGACTGGTACTTCGATCTCTGGGGCCG |
| | | | TGGCACCCTGGTCACTGTCTCCTCAGCTAGCACTAAAGGGCCTTCT |
| | | | GTATTTCCCTTGGCCCCGTCCAGCAAATCGACCTCGGGAGGGACA |
| | | | GCCGCCCTGGGTTGCCTTGTGAAAGATTATTTCCCTGAGCCAGTTA |
| | | | CCGTAAGTTGGAACAGTGGGGCGCTGACAAGTGGTGTGCACACGT |
| | | | TTCCTGCCGTCCTGCAATCATCGGGCTTGTATAGCCTCAGCTCTGT |
| | | | GGTCACTGTCCCAAGTTCATCGCTGGGCACTCAGACGTATATTTGC |
| | | | AATGTGAACCACAAACCTTCAAATACAAAAGTGGATAAACGCGTA |
| | | | GAACCGAAATCGTGTGATAAAACTCACACATGCCCGCCATGCCCG |
| | | | GCACCTGAACTGCTTGGTGGTCCCAGCGTGTTCCTGTTCCCGCCGA |
| | | | AGCCTAAAGATACTCTAATGATCAGCCGTACGCCAGAGGTGACAT |
| | | | GTGTCGTGGTTGACGTGTCCCACGAAGATCCCGAAGTTAAGTTCA |
| | | | ATTGGTATGTTGATGGTGTAGAGGTACACAATGCTAAGACTAAAC |
| | | | CTCGCGAGGAGCAGTACGCCTCGACCTATCGTGTCGTGAGCGTTCT |
| | | | GACCGTCCTTCACCAAGATTGGCTTAACGGCAAAGAATATAAGTG |
| | | | CAAGGTAAGCAATAAAGCACTTCCGGCCCCAATCGAGAAAACCAT |
| | | | TTCCAAGGCCAAAGGTCAACCAAGAGAACCCCAGGTGTATACTCT |
| | | | TCCGCCTTCTCGTGAGGAAATGACTAAAAATCAAGTATCCCTTACG |
| | | | TGTCTGGTTAAAGGTTTTTATCCTAGCGATATTGCTGTTGAATGGG |
| | | | AATCGAACGGTCAGCCGGAGAATAATTATAAAACAACGCCACCCG |
| | | | TCCTGGATAGCGACGGCTCATTTTTTCTGTATAGCAAACTGACTGT |
| | | | AGATAAATCACGGTGGCAGCAGGGCAATGTATTCAGTTGCTCCGT |
| | | | TATGCATGAAGCGTTACATAATCACTACACGCAGAAATCTCTTAGT |
| | | | CTTTCACCCGGT |
| 499 | ATX-P-582 | HC IgG1 Fc- | CAGGTGCAGCTGCAGGAGTCGGGCCCAGGACTGGTGAAGCCTTCG |
| | | nucleotide | GGGACCCTGTCCCTCACCTGCGCTGTGTCTGGTGGCTCCATCAGCA |
| | | | TTAGTAAATGGTGGAGTTGGGTCCGCCAGCCCCCAGGGAAGGGAC |
| | | | TGGAGTGGATTGGGGAAATCTATCATACTGGGAGCACCAACTACC |
| | | | ACCCGTCCCTCAAGAGCCGAGTCACCATATCAGTAGACAAGTCCA |
| | | | AGAACCAATTCTACCTGAAGCTGAACTCTGTGACCGCCGCGGACA |
| | | | CGGCCGTATATTACTGTGCGAGAGATGAATTACGGTTGGGGGAGT |
| | | | TATCGCCCTCTCTTGATATCTGGGGACAAGGGACAATGGTCACCGT |
| | | | CTCTTCAGCTAGCACTAAAGGGCCTTCTGTATTTCCCTTGGCCCCG |
| | | | TCCAGCAAATCGACCTCGGGAGGGACAGCCGCCCTGGGTTGCCTT |
| | | | GTGAAAGATTATTTCCCTGAGCCAGTTACCGTAAGTTGGAACAGT |
| | | | GGGGCGCTGACAAGTGGTGTGCACACGTTTCCTGCCGTCCTGCAAT |
| | | | CATCGGGCTTGTATAGCCTCAGCTCTGTGGTCACTGTCCCAAGTTC |
| | | | ATCGCTGGGCACTCAGACGTATATTTGCAATGTGAACCACAAACC |
| | | | TTCAAATACAAAAGTGGATAAACGCGTAGAACCGAAATCGTGTGA |
| | | | TAAAACTCACACATGCCCGCCATGCCCGGCACCTGAACTGCTTGGT |
| | | | GGTCCCAGCGTGTTCCTGTTCCCGCCGAAGCCTAAAGATACTCTAA |
| | | | TGATCAGCCGTACGCCAGAGGTGACATGTGTCGTGGTTGACGTGT |
| | | | CCCACGAAGATCCCGAAGTTAAGTTCAATTGGTATGTTGATGGTGT |
| | | | AGAGGTACACAATGCTAAGACTAAACCTCGCGAGGAGCAGTACGC |
| | | | CTCGACCTATCGTGTCGTGAGCGTTCTGACCGTCCTTCACCAAGAT |
| | | | TGGCTTAACGGCAAAGAATATAAGTGCAAGGTAAGCAATAAAGCA |
| | | | CTTCCGGCCCCAATCGAGAAAACCATTTCCAAGGCCAAAGGTCAA |
| | | | CCAAGAGAACCCCAGGTGTATACTCTTCCGCCTTCTCGTGAGGAA |
| | | | ATGACTAAAAATCAAGTATCCCTTACGTGTCTGGTTAAAGGTTTTT |
| | | | ATCCTAGCGATATTGCTGTTGAATGGGAATCGAACGGTCAGCCGG |
| | | | AGAATAATTATAAAACAACGCCACCCGTCCTGGATAGCGACGGCT |
| | | | CATTTTTTCTGTATAGCAAACTGACTGTAGATAAATCACGGTGGCA |
| | | | GCAGGGCAATGTATTCAGTTGCTCCGTTATGCATGAAGCGTTACAT |
| | | | AATCACTACACGCAGAAATCTCTTAGTCTTTCACCCGGT |
| 500 | ATX-P-598 | HC IgG1 Fc- | CAGGTGCAGCTGCAGGAGTCGGGCCCAGGACTGGTGAAGCCTTCG |
| | | nucleotide | GGGACCCTGTCCCTCACCTGCGCTGTCTCTGGTGGCTCCATCAGCA |
| | | | GTAGTAACTGGTGGAGTTGGGTCCGCCAGTCCCCAGGGAAGGGGC |
| | | | TGGAATGGATTGGGGAAATCTATCATAGTGGGACCACCAACTACA |
| | | | ACCCGTCCCTCAAGAGTCGAGTCACCATATCAGTAGACAAGTCCA |
| | | | AGAACCAGTTCTCCCTGAAGCTGAGCTCTGCGACCGCCGCGGACA |
| | | | CGGCCGTGTATTATTGTGCGAGAGATAGATTACGTTTGGGGGAGT |
| | | | CATCGTCCGATGTTTTTGATATCTGGGGACAAGGGACAATGGTCAC |
| | | | CGTCTCTTCAGCTAGCACTAAAGGGCCTTCTGTATTTCCCTTGGCC |
| | | | CCGTCCAGCAAATCGACCTCGGGAGGGACAGCCGCCCTGGGTTGC |
| | | | CTTGTGAAAGATTATTTCCCTGAGCCAGTTACCGTAAGTTGGAACA |
| | | | GTGGGGCGCTGACAAGTGGTGTGCACACGTTTCCTGCCGTCCTGCA |
| | | | ATCATCGGGCTTGTATAGCCTCAGCTCTGTGGTCACTGTCCCAAGT |
| | | | TCATCGCTGGGCACTCAGACGTATATTTGCAATGTGAACCACAAA |
| | | | CCTTCAAATACAAAAGTGGATAAACGCGTAGAACCGAAATCGTGT |
| | | | GATAAAACTCACACATGCCCGCCATGCCCGGCACCTGAACTGCTT |
| | | | GGTGGTCCCAGCGTGTTCCTGTTCCCGCCGAAGCCTAAAGATACTC |
| | | | TAATGATCAGCCGTACGCCAGAGGTGACATGTGTCGTGGTTGACG |
| | | | TGTCCCACGAAGATCCCGAAGTTAAGTTCAATTGGTATGTTGATGG |
| | | | TGTAGAGGTACACAATGCTAAGACTAAACCTCGCGAGGAGCAGTA |
| | | | CGCCTCGACCTATCGTGTCGTGAGCGTTCTGACCGTCCTTCACCAA |
| | | | GATTGGCTTAACGGCAAAGAATATAAGTGCAAGGTAAGCAATAAA |
| | | | GCACTTCCGGCCCCAATCGAGAAAACCATTTCCAAGGCCAAAGGT |
| | | | CAACCAAGAGAACCCCAGGTGTATACTCTTCCGCCTTCTCGTGAGG |
| | | | AAATGACTAAAAATCAAGTATCCCTTACGTGTCTGGTTAAAGGTTT |
| | | | TTATCCTAGCGATATTGCTGTTGAATGGGAATCGAACGGTCAGCCG |
| | | | GAGAATAATTATAAAACAACGCCACCCGTCCTGGATAGCGACGGC |
| | | | TCATTTTTTCTGTATAGCAAACTGACTGTAGATAAATCACGGTGGC |
| | | | AGCAGGGCAATGTATTCAGTTGCTCCGTTATGCATGAAGCGTTACA |
| | | | TAATCACTACACGCAGAAATCTCTTAGTCTTTCACCCGGT |
| 501 | ATX-P-603 | HC IgG1 Fc- | CAGGTGCAGCTGCAGGAGTCGGGCCCAGGACTGGTGAAGCCTTCG |
| | | nucleotide | GGGACCCTGTCCCTCACCTGCGCTGTCTCTGGTGGCTCCATCAGCG |
| | | | GTAGTCACTGGTGGAGTTGGTTCCGCCAGCCCCCAGGAAAGGGGC |
| | | | TGGAGTGGATTGGGGAAATCTATCATACTGGGAGCACCAACAAAA |
| | | | ACCCGTCCCTCAAGAGTCGAGTCACTATCTCATTAGACAAGTCCAA |
| | | | GAATCAGTTCTCCCTGAGGCTGAGGTCTGTGACCGACGCGGACAC |
| | | | GGCCGTGTATTACTGTGCGAGAGATCAGTATACTGGGAGCCCAAG |
| | | | GGATGCTTTTGATATCTGGGGACAAGGGACAATGGTCACCGTCTC |
| | | | CTCAGCTAGCACTAAAGGGCCTTCTGTATTTCCCTTGGCCCCGTCC |
| | | | AGCAAATCGACCTCGGGAGGGACAGCCGCCCTGGGTTGCCTTGTG |
| | | | AAAGATTATTTCCCTGAGCCAGTTACCGTAAGTTGGAACAGTGGG |
| | | | GCGCTGACAAGTGGTGTGCACACGTTTCCTGCCGTCCTGCAATCAT |
| | | | CGGGCTTGTATAGCCTCAGCTCTGTGGTCACTGTCCCAAGTTCATC |
| | | | GCTGGGCACTCAGACGTATATTTGCAATGTGAACCACAAACCTTC |
| | | | AAATACAAAAGTGGATAAACGCGTAGAACCGAAATCGTGTGATAA |
| | | | AACTCACACATGCCCGCCATGCCCGGCACCTGAACTGCTTGGTGGT |
| | | | CCCAGCGTGTTCCTGTTCCCGCCGAAGCCTAAAGATACTCTAATGA |
| | | | TCAGCCGTACGCCAGAGGTGACATGTGTCGTGGTTGACGTGTCCC |
| | | | ACGAAGATCCCGAAGTTAAGTTCAATTGGTATGTTGATGGTGTAG |
| | | | AGGTACACAATGCTAAGACTAAACCTCGCGAGGAGCAGTACGCCT |
| | | | CGACCTATCGTGTCGTGAGCGTTCTGACCGTCCTTCACCAAGATTG |
| | | | GCTTAACGGCAAAGAATATAAGTGCAAGGTAAGCAATAAAGCACT |
| | | | TCCGGCCCCAATCGAGAAAACCATTTCCAAGGCCAAAGGTCAACC |
| | | | AAGAGAACCCCAGGTGTATACTCTTCCGCCTTCTCGTGAGGAAAT |
| | | | GACTAAAAATCAAGTATCCCTTACGTGTCTGGTTAAAGGTTTTTAT |
| | | | CCTAGCGATATTGCTGTTGAATGGGAATCGAACGGTCAGCCGGAG |
| | | | AATAATTATAAAACAACGCCACCCGTCCTGGATAGCGACGGCTCA |
| | | | TTTTTTCTGTATAGCAAACTGACTGTAGATAAATCACGGTGGCAGC |
| | | | AGGGCAATGTATTCAGTTGCTCCGTTATGCATGAAGCGTTACATAA |
| | | | TCACTACACGCAGAAATCTCTTAGTCTTTCACCCGGT |
| 502 | ATX-P-608 | HC IgG1 Fc- | CAGGTGCAGCTGCAGGAGTCGGGCCCAGGACTGGTGAAGCCTTCG |
| | | nucleotide | GGGACCCTGTCCCTCACCTGCGCTGTCTCTGGTGGCTCCATCAGCA |
| | | | GTAGTAACTGGTGGAGTTGGGTCCGCCAGTCCCCAGGGAAGGGGC |
| | | | TGGAGTGGATTGGGGAAATCTATCATACTGGGAGCACCAACAAAA |
| | | | AACCGTCCCTCAAGAGTCGAGTCATCATATCAGTAGACAAGTCCA |
| | | | AGAACCAGTTCTCCCTGAAACTGATCTCTGTGAACGCCGCGGACA |
| | | | CGGCCATATATTACTGTGCGAGAGATCGGGAAGGGGGAACTACTG |
| | | | AAGCTTTTGATATCTGGGGACAAGGGACAATGGTCACCGTCTCTTC |
| | | | AGCTAGCACTAAAGGGCCTTCTGTATTTCCCTTGGCCCCGTCCAGC |
| | | | AAATCGACCTCGGGAGGGACAGCCGCCCTGGGTTGCCTTGTGAAA |
| | | | GATTATTTCCCTGAGCCAGTTACCGTAAGTTGGAACAGTGGGGCG |
| | | | CTGACAAGTGGTGTGCACACGTTTCCTGCCGTCCTGCAATCATCGG |
| | | | GCTTGTATAGCCTCAGCTCTGTGGTCACTGTCCCAAGTTCATCGCT |
| | | | GGGCACTCAGACGTATATTTGCAATGTGAACCACAAACCTTCAAA |
| | | | TACAAAAGTGGATAAACGCGTAGAACCGAAATCGTGTGATAAAAC |
| | | | TCACACATGCCCGCCATGCCCGGCACCTGAACTGCTTGGTGGTCCC |
| | | | AGCGTGTTCCTGTTCCCGCCGAAGCCTAAAGATACTCTAATGATCA |
| | | | GCCGTACGCCAGAGGTGACATGTGTCGTGGTTGACGTGTCCCACG |
| | | | AAGATCCCGAAGTTAAGTTCAATTGGTATGTTGATGGTGTAGAGG |
| | | | TACACAATGCTAAGACTAAACCTCGCGAGGAGCAGTACGCCTCGA |
| | | | CCTATCGTGTCGTGAGCGTTCTGACCGTCCTTCACCAAGATTGGCT |
| | | | TAACGGCAAAGAATATAAGTGCAAGGTAAGCAATAAAGCACTTCC |
| | | | GGCCCCAATCGAGAAAACCATTTCCAAGGCCAAAGGTCAACCAAG |
| | | | AGAACCCCAGGTGTATACTCTTCCGCCTTCTCGTGAGGAAATGACT |
| | | | AAAAATCAAGTATCCCTTACGTGTCTGGTTAAAGGTTTTTATCCTA |
| | | | GCGATATTGCTGTTGAATGGGAATCGAACGGTCAGCCGGAGAATA |
| | | | ATTATAAAACAACGCCACCCGTCCTGGATAGCGACGGCTCATTTTT |
| | | | TCTGTATAGCAAACTGACTGTAGATAAATCACGGTGGCAGCAGGG |
| | | | CAATGTATTCAGTTGCTCCGTTATGCATGAAGCGTTACATAATCAC |
| | | | TACACGCAGAAATCTCTTAGTCTTTCACCCGGT |
| 503 | ATX-P-609 | HC IgG1 Fc- | CAGGTGCAGCTGCAGGAGTCGGGCCCAGGACTGGTGAAGCCTTCG |
| | | nucleotide | GGGACCCTGTCCCTCACCTGCGCTGTCTCTGGTGGCTCCATCAGCA |
| | | | GTAGTAACTGGTGGAGTTGGGTCCGCCAGCCCCCAGGGAAGGGGC |
| | | | TGGAGTGGATTGGGGAAATCTATCATAGTGGGAGCACCAACTACA |
| | | | ACCCGTCCCTCAAGAGTCGAGTCACCATATCAGTAGACAAGTCCA |
| | | | AGAACCAGTTCTCCCTGAAGCTGAGGTCTGTGACCGCCGCGGACA |
| | | | CGGCCGTTTATTACTGTGCGAGAGATCAACTAAGTAGCGGACCTA |
| | | | CGGATGCTTTTGATATCTGGGGACAAGGGACAATGGTCACCGTCT |
| | | | CTTCAGCTAGCACTAAAGGGCCTTCTGTATTTCCCTTGGCCCCGTC |
| | | | CAGCAAATCGACCTCGGGAGGGACAGCCGCCCTGGGTTGCCTTGT |
| | | | GAAAGATTATTTCCCTGAGCCAGTTACCGTAAGTTGGAACAGTGG |
| | | | GGCGCTGACAAGTGGTGTGCACACGTTTCCTGCCGTCCTGCAATCA |
| | | | TCGGGCTTGTATAGCCTCAGCTCTGTGGTCACTGTCCCAAGTTCAT |
| | | | CGCTGGGCACTCAGACGTATATTTGCAATGTGAACCACAAACCTTC |
| | | | AAATACAAAAGTGGATAAACGCGTAGAACCGAAATCGTGTGATAA |
| | | | AACTCACACATGCCCGCCATGCCCGGCACCTGAACTGCTTGGTGGT |
| | | | CCCAGCGTGTTCCTGTTCCCGCCGAAGCCTAAAGATACTCTAATGA |
| | | | TCAGCCGTACGCCAGAGGTGACATGTGTCGTGGTTGACGTGTCCC |
| | | | ACGAAGATCCCGAAGTTAAGTTCAATTGGTATGTTGATGGTGTAG |
| | | | AGGTACACAATGCTAAGACTAAACCTCGCGAGGAGCAGTACGCCT |
| | | | CGACCTATCGTGTCGTGAGCGTTCTGACCGTCCTTCACCAAGATTG |
| | | | GCTTAACGGCAAAGAATATAAGTGCAAGGTAAGCAATAAAGCACT |
| | | | TCCGGCCCCAATCGAGAAAACCATTTCCAAGGCCAAAGGTCAACC |
| | | | AAGAGAACCCCAGGTGTATACTCTTCCGCCTTCTCGTGAGGAAAT |
| | | | GACTAAAAATCAAGTATCCCTTACGTGTCTGGTTAAAGGTTTTTAT |
| | | | CCTAGCGATATTGCTGTTGAATGGGAATCGAACGGTCAGCCGGAG |
| | | | AATAATTATAAAACAACGCCACCCGTCCTGGATAGCGACGGCTCA |
| | | | TTTTTTCTGTATAGCAAACTGACTGTAGATAAATCACGGTGGCAGC |
| | | | AGGGCAATGTATTCAGTTGCTCCGTTATGCATGAAGCGTTACATAA |
| | | | TCACTACACGCAGAAATCTCTTAGTCTTTCACCCGGT |
| 504 | ATX-P-563 | HC IgG1 Fc- | CAGGTGCAGCTGCAGGAGTCGGGCCCAGGACTGATGAAGCCTTCG |
| | | nucleotide | GAGACCCTGTCCCTCACCTGCACTGTCTCTGGTGGCTCCATCAGTA |
| | | | GTCACTACTGGAGCTGGATCCGGCAACCCCCAGGGAAGGGACTGG |
| | | | AGTGGATTGGATATATCTTTTACAGTGGGAACATAAATTACAACCC |
| | | | CTCCCTCAAGAGTCGAGTCACCATATCAGTAGACACGTCCAAGAA |
| | | | CCAGTTCTCCCTGAAGCTGAGGTCTGTGACCACTGCGGACACGGC |
| | | | CGTATATTACTGTGCGAGAGATCGAGGTGGGAGCTATTCGTACCA |
| | | | CTTTGACTGCTGGGGCCAGGGAACCCTGGTCACCGTCTCCTCAGCT |
| | | | AGCACTAAAGGGCCTTCTGTATTTCCCTTGGCCCCGTCCAGCAAAT |
| | | | CGACCTCGGGAGGGACAGCCGCCCTGGGTTGCCTTGTGAAAGATT |
| | | | ATTTCCCTGAGCCAGTTACCGTAAGTTGGAACAGTGGGGCGCTGA |
| | | | CAAGTGGTGTGCACACGTTTCCTGCCGTCCTGCAATCATCGGGCTT |
| | | | GTATAGCCTCAGCTCTGTGGTCACTGTCCCAAGTTCATCGCTGGGC |
| | | | ACTCAGACGTATATTTGCAATGTGAACCACAAACCTTCAAATACA |
| | | | AAAGTGGATAAACGCGTAGAACCGAAATCGTGTGATAAAACTCAC |
| | | | ACATGCCCGCCATGCCCGGCACCTGAACTGCTTGGTGGTCCCAGC |
| | | | GTGTTCCTGTTCCCGCCGAAGCCTAAAGATACTCTAATGATCAGCC |
| | | | GTACGCCAGAGGTGACATGTGTCGTGGTTGACGTGTCCCACGAAG |
| | | | ATCCCGAAGTTAAGTTCAATTGGTATGTTGATGGTGTAGAGGTACA |
| | | | CAATGCTAAGACTAAACCTCGCGAGGAGCAGTACGCCTCGACCTA |
| | | | TCGTGTCGTGAGCGTTCTGACCGTCCTTCACCAAGATTGGCTTAAC |
| | | | GGCAAAGAATATAAGTGCAAGGTAAGCAATAAAGCACTTCCGGCC |
| | | | CCAATCGAGAAAACCATTTCCAAGGCCAAAGGTCAACCAAGAGAA |
| | | | CCCCAGGTGTATACTCTTCCGCCTTCTCGTGAGGAAATGACTAAAA |
| | | | ATCAAGTATCCCTTACGTGTCTGGTTAAAGGTTTTTATCCTAGCGA |
| | | | TATTGCTGTTGAATGGGAATCGAACGGTCAGCCGGAGAATAATTA |
| | | | TAAAACAACGCCACCCGTCCTGGATAGCGACGGCTCATTTTTTCTG |
| | | | TATAGCAAACTGACTGTAGATAAATCACGGTGGCAGCAGGGCAAT |
| | | | GTATTCAGTTGCTCCGTTATGCATGAAGCGTTACATAATCACTACA |
| | | | CGCAGAAATCTCTTAGTCTTTCACCCGGT |
| 505 | ATX-P-570 | HC IgG1 Fc- | CAGGTGCAGCTGCAGGAGTCGGGCCCAGGACTGGTGAAGCCTTCG |
| | | nucleotide | GAGACTATGTCCCTCACCTGCACTGTCTCTGGTGGCTCCTTCACTG |
| | | | GATACTACTGGAGTTGGATCCGGCAGCCCCCAGGGAAGGGACTGG |
| | | | AGTGGATTGGATATGTCCATTACAGTGGGGGCACCAAAAGCAACC |
| | | | CCTCCCTCAAGAGTCGAGTCACCATATCAGTAGACACGTCCAAGA |
| | | | ACCAGTTTTCCCTGAAGCTGAGCTCTGTGACCGCTGCGGACACGGC |
| | | | CGTGTATTACTGTGCGAGAGAGAGGTATGAGTGGAACTCCTACTTT |
| | | | GACTACTGGGGCCAGGGAACCCTGGTCACCGTCTCCTCAGCTAGC |
| | | | ACTAAAGGGCCTTCTGTATTTCCCTTGGCCCCGTCCAGCAAATCGA |
| | | | CCTCGGGAGGGACAGCCGCCCTGGGTTGCCTTGTGAAAGATTATTT |
| | | | CCCTGAGCCAGTTACCGTAAGTTGGAACAGTGGGGCGCTGACAAG |
| | | | TGGTGTGCACACGTTTCCTGCCGTCCTGCAATCATCGGGCTTGTAT |
| | | | AGCCTCAGCTCTGTGGTCACTGTCCCAAGTTCATCGCTGGGCACTC |
| | | | AGACGTATATTTGCAATGTGAACCACAAACCTTCAAATACAAAAG |
| | | | TGGATAAACGCGTAGAACCGAAATCGTGTGATAAAACTCACACAT |
| | | | GCCCGCCATGCCCGGCACCTGAACTGCTTGGTGGTCCCAGCGTGTT |
| | | | CCTGTTCCCGCCGAAGCCTAAAGATACTCTAATGATCAGCCGTACG |
| | | | CCAGAGGTGACATGTGTCGTGGTTGACGTGTCCCACGAAGATCCC |
| | | | GAAGTTAAGTTCAATTGGTATGTTGATGGTGTAGAGGTACACAAT |
| | | | GCTAAGACTAAACCTCGCGAGGAGCAGTACGCCTCGACCTATCGT |
| | | | GTCGTGAGCGTTCTGACCGTCCTTCACCAAGATTGGCTTAACGGCA |
| | | | AAGAATATAAGTGCAAGGTAAGCAATAAAGCACTTCCGGCCCCAA |
| | | | TCGAGAAAACCATTTCCAAGGCCAAAGGTCAACCAAGAGAACCCC |
| | | | AGGTGTATACTCTTCCGCCTTCTCGTGAGGAAATGACTAAAAATCA |
| | | | AGTATCCCTTACGTGTCTGGTTAAAGGTTTTTATCCTAGCGATATT |
| | | | GCTGTTGAATGGGAATCGAACGGTCAGCCGGAGAATAATTATAAA |
| | | | ACAACGCCACCCGTCCTGGATAGCGACGGCTCATTTTTTCTGTATA |
| | | | GCAAACTGACTGTAGATAAATCACGGTGGCAGCAGGGCAATGTAT |
| | | | TCAGTTGCTCCGTTATGCATGAAGCGTTACATAATCACTACACGCA |
| | | | GAAATCTCTTAGTCTTTCACCCGGT |
| 506 | ATX-P-571 | LC Kappa | DIQMTQSPSSLSASVGDRVTITCRASQSISSYLNWYQQKPGKAPKVLI |
| | | | YAASSLQSGIPSRFSGSGSGTDFTLTISSLQPEDFATYYCQETYSVPLTF |
| | | | GGGTKVEIKRTVAAPSVFIFPPSDEQLKSGTASVVCLLNNFYPREAKV |
| | | | QWKVDNALQSGNSQESVTEQDSKDSTYSLSSTLTLSKADYEKHKVY |
| | | | ACEVTHQGLSSPVTKSFNRGEC |
| 507 | ATX-P-577 | LC Kappa | DIQMTQSPSSLSASVGDRVTITCRASQDIRNDLGWYQQKPGKVPKRLI |
| | | | YAASSLQSGVPSRFSGSGSGTEFTLTISSLQPEDFASYYCLQYNNYPFT |
| | | | LGPGTKVDIKRTVAAPSVFIFPPSDEQLKSGTASVVCLLNNFYPREAK |
| | | | VQWKVDNALQSGNSQESVTEQDSKDSTYSLSSTLILSKADYEKHKV |
| | | | YACEVTHQGLSSPVTKSFNRGEC |
| 508 | ATX-P-579 | LC Kappa | DIQMTQSPSSLSASVGDRVTITCRASQGIRKDLGWYQQKPGKAPKRLI |
| | | | YTASTLQSGVPSRFSGSGSGTEFTLTISSLQPEDFATYYCLQYNNYPFT |
| | | | FGPGTKVDIKRTVAAPSVFIFPPSDEQLKSGTASVVCLLNNFYPREAK |
| | | | VQWKVDNALQSGNSQESVTEQDSKDSTYSLSSTLTLSKADYEKHKV |
| | | | YACEVTHQGLSSPVTKSFNRGEC |
| 509 | ATX-P-580 | LC Kappa | DIQMTQSPSSLSASVGDRVTITCRASQGIRNDLGWYQQKPGKAPKRLI |
| | | | YAASSLQSGVPSRFSGSGSGTEFTLAISSLQPEDFATYYCLHFNSYPFT |
| | | | FGPGTKVDIKRTVAAPSVFIFPPSDEQLKSGTASVVCLLNNFYPREAK |
| | | | VQWKVDNALQSGNSQESVTEQDSKDSTYSLSSTLTLSKADYEKHKV |
| | | | YACEVTHQGLSSPVTKSFNRGEC |
| 510 | ATX-P-582 | LC Kappa | DIQMTQSPSSLSASVGDRVTITCRASQNINNYLNWFRQKPGKAPKLLI |
| | | | FAASSLQSGVPSRFSGSESGTDFTLTISSLQPEDFATYYCQQSYSTPLTF |
| | | | GGGTKVEIKRTVAAPSVFIFPPSDEQLKSGTASVVCLLNNFYPREAKV |
| | | | QWKVDNALQSGNSQESVTEQDSKDSTYSLSSTLTLSKADYEKHKVY |
| | | | ACEVTHQGLSSPVTKSFNRGEC |
| 511 | ATX-P-598 | LC Kappa | DIVMTQSPDSLAVSLGERATINCKSSQSVLYSSNNKNYLAWYQQKPG |
| | | | QPPELLIYWASTRESGVPDRFSGSGSGTDFTLTISSLQAEDMAVYYCQ |
| | | | QFYNTPYTFGQGTKVDIKRTVAAPSVFIFPPSDEQLKSGTASVVCLLN |
| | | | NFYPREAKVQWKVDNALQSGNSQESVTEQDSKDSTYSLSSTLTLSKA |
| | | | DYEKHKVYACEVTHQGLSSPVTKSFNRGEC |
| 512 | ATX-P-603 | LC Kappa | EIVLTQSPGTLSLSPGERATLSCRASQSVSSSYLAWYQQKPGQAPRLLI |
| | | | YGASSRATGIPDRFSGSGSGTDFTLTISRLEPEDFAVYYCQHYGNSPLT |
| | | | FGGGTKVEIKRTVAAPSVFIFPPSDEQLKSGTASVVCLLNNFYPREAK |
| | | | VQWKVDNALQSGNSQESVTEQDSKDSTYSLSSTLTLSKADYEKHKV |
| | | | YACEVTHQGLSSPVTKSFNRGEC |
| 513 | ATX-P-608 | LC Kappa | EIVMTQSPATLSVSPGERATLSCRASQSVSSNLAWYQQKPGQAPRLLI |
| | | | YGASTRATGIPVRFSGSGSGTQFTLTISSLQSEDFAVYYCQQYDNWRT |
| | | | FGQGTRLEIKRTVAAPSVFIFPPSDEQLKSGTASVVCLLNNFYPREAKV |
| | | | QWKVDNALQSGNSQESVTEQDSKDSTYSLSSTLTLSKADYEKHKVY |
| | | | ACEVTHQGLSSPVTKSFNRGEC |
| 514 | ATX-P-609 | LC Kappa | EIVMTQSPATLSVSPGERATLSCRASQSVSSNLAWYQQKPGQAPRLLI |
| | | | YGASTRATGIPVRFSGSGSGTQFTLTISSLQSEDFAVYYCQQYDNWRT |
| | | | FGQGTKLEIKRTVAAPSVFIFPPSDEQLKSGTASVVCLLNNFYPREAK |
| | | | VQWKVDNALQSGNSQESVTEQDSKDSTYSLSSTLTLSKADYEKHKV |
| | | | YACEVTHQGLSSPVTKSFNRGEC |
| 515 | ATX-P-563 | LC Kappa | DIQMTQSPSSVSASVGDRVTITCRASQSFAGWLAWYQQKPGKAPKLL |
| | | | IYAASTLQSGVPSRFSGSGSGTDFTLTISSLQPEDFATYYCQQANSFPL |
| | | | TFGGGTKVEIKRTVAAPSVFIFPPSDEQLKSGTASVVCLLNNFYPREA |
| | | | KVQWKVDNALQSGNSQESVTEQDSKDSTYSLSSTLTLSKADYEKHK |
| | | | VYACEVTHQGLSSPVIKSFNRGEC |
| 516 | ATX-P-570 | LC Kappa | DIVMTQSPDSLAVSLGERATINCKSSQSVLYSSNNKNYLAWYHQKPG |
| | | | QPPKLLIYWASTRESGVPDRFSGSGSGTDFTLTISSLQAEDVAVYYCQ |
| | | | QYYSSPRTFGQGTKVEIKRTVAAPSVFIFPPSDEQLKSGTASVVCLLNN |
| | | | FYPREAKVQWKVDNALQSGNSQESVTEQDSKDSTYSLSSTLTLSKAD |
| | | | YEKHKVYACEVTHQGLSSPVTKSFNRGEC |
| 517 | ATX-P-571 | LC Kappa- | GACATCCAGATGACCCAGTCTCCATCCTCCCTGTCTGCATCTGTGG |
| | | nucleotide | GAGACAGAGTCACCATCACTTGCCGGGCAAGTCAGAGCATTAGCA |
| | | | GCTATTTAAATTGGTATCAGCAGAAACCAGGGAAAGCCCCTAAGG |
| | | | TCCTGATCTATGCTGCATCCAGTTTGCAGAGTGGGATCCCATCACG |
| | | | GTTCAGTGGCAGTGGATCTGGGACAGATTTCACTCTCACCATCAGC |
| | | | AGTCTGCAACCTGAAGATTTTGCAACTTACTACTGTCAAGAGACTT |
| | | | ACAGTGTTCCGCTCACTTTCGGCGGAGGGACCAAGGTGGAGATCA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 518 | ATX-P-577 | LC Kappa- | GACATCCAGATGACCCAGTCTCCATCCTCCCTGTCTGCATCTGTAG |
| | | nucleotide | GAGACAGAGTCACCATCACTTGCCGGGCAAGTCAGGACATTAGAA |
| | | | ATGATTTAGGCTGGTATCAGCAGAAACCAGGGAAAGTCCCTAAGC |
| | | | GCCTGATCTATGCTGCATCTAGTTTGCAAAGTGGGGTCCCATCAAG |
| | | | GTTCAGCGGCAGTGGATCTGGGACAGAATTCACTCTCACAATCAG |
| | | | CAGCCTGCAGCCTGAAGATTTCGCAAGTTATTACTGTCTACAGTAT |
| | | | AATAATTATCCATTCACTCTCGGCCCTGGGACCAAAGTGGATATCA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 519 | ATX-P-579 | LC Kappa- | GACATCCAGATGACCCAGTCTCCATCCTCCCTGTCTGCATCTGTAG |
| | | nucleotide | GAGACAGAGTCACCATCACTTGCCGGGCAAGTCAGGGCATTAGAA |
| | | | AAGATTTAGGCTGGTATCAGCAGAAACCAGGGAAAGCCCCTAAGC |
| | | | GCCTGATCTATACTGCATCCACTTTACAAAGTGGGGTCCCATCAAG |
| | | | GTTCAGCGGCAGTGGATCTGGGACAGAATTCACTCTCACAATCAG |
| | | | CAGCCTGCAGCCTGAAGATTTTGCAACTTATTACTGTCTACAGTAT |
| | | | AATAATTACCCATTCACTTTCGGCCCTGGGACCAAAGTGGATATCA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 520 | ATX-P-580 | LC Kappa- | GACATCCAGATGACCCAGTCTCCATCCTCCCTGTCTGCTTCTGTCG |
| | | nucleotide | GAGACAGAGTCACCATCACTTGCCGGGCAAGTCAGGGCATTAGAA |
| | | | ATGATTTAGGCTGGTATCAGCAGAAACCAGGGAAAGCCCCTAAGC |
| | | | GCCTGATCTATGCTGCATCCAGTTTGCAAAGTGGGGTCCCATCAAG |
| | | | GTTCAGCGGCAGTGGATCTGGGACAGAATTCACTCTCGCAATCAG |
| | | | CAGCCTGCAGCCTGAAGATTTTGCAACTTATTACTGTCTACACTTT |
| | | | AATAGTTACCCATTCACTTTCGGCCCTGGGACCAAAGTGGATATCA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 521 | ATX-P-582 | LC Kappa- | GACATCCAGATGACCCAGTCTCCATCCTCCCTGTCTGCATCTGTAG |
| | | nucleotide | GAGACAGAGTCACCATCACTTGCCGGGCAAGTCAGAACATTAACA |
| | | | ACTATTTAAATTGGTTCCGGCAGAAACCAGGGAAAGCCCCTAAGC |
| | | | TCCTGATCTTTGCTGCATCCAGTTTACAAAGTGGGGTCCCATCAAG |
| | | | ATTCAGTGGCAGTGAATCTGGGACAGATTTCACTCTCACCATCAGC |
| | | | AGTCTGCAACCTGAGGATTTTGCGACTTACTACTGTCAACAGAGTT |
| | | | ACAGTACCCCTCTCACTTTCGGCGGAGGGACCAAGGTGGAGATCA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 522 | ATX-P-598 | LC Kappa- | GACATCGTGATGACTCAGTCTCCAGACTCCCTGGCTGTGTCTCTGG |
| | | nucleotide | GCGAGAGGGCCACCATCAACTGCAAGTCCAGCCAGAGTGTTTTAT |
| | | | ACAGCTCCAACAATAAGAACTACTTAGCTTGGTACCAGCAGAAAC |
| | | | CAGGACAGCCTCCTGAGCTGCTCATTTACTGGGCATCTACCCGGGA |
| | | | ATCCGGGGTCCCTGACCGATTCAGTGGCAGCGGGTCTGGGACAGA |
| | | | TTTCACTCTCACCATCAGCAGCCTGCAGGCTGAAGATATGGCAGTT |
| | | | TATTACTGTCAACAATTTTATAATACTCCGTACACTTTTGGCCAGG |
| | | | GGACCAAAGTGGATATCAAACGTACGGTAGCTGCCCCTTCAGTTTT |
| | | | TATCTTTCCGCCGTCTGACGAGCAGTTAAAATCCGGGACCGCTTCT |
| | | | GTAGTTTGCCTGCTGAATAATTTTTATCCGCGTGAGGCTAAAGTAC |
| | | | AATGGAAAGTCGACAATGCTTTGCAGTCGGGAAATTCACAGGAAA |
| | | | GTGTTACGGAGCAGGATTCTAAAGATTCCACATATTCACTCAGCTC |
| | | | CACCCTTACACTGAGCAAAGCCGACTATGAAAAACATAAAGTTTA |
| | | | CGCATGTGAGGTGACGCACCAAGGATTATCCAGTCCGGTCACAAA |
| | | | ATCGTTTAACCGCGGTGAGTGT |
| 523 | ATX-P-603 | LC Kappa- | GAAATTGTGTTGACGCAGTCTCCAGGCACCCTGTCTTTGTCTCCAG |
| | | nucleotide | GGGAAAGAGCCACCCTCTCCTGCAGGGCCAGTCAGAGTGTTAGCA |
| | | | GCAGCTACTTAGCCTGGTACCAGCAGAAACCTGGCCAGGCTCCCA |
| | | | GGCTCCTCATCTATGGTGCATCCAGCAGGGCCACTGGCATCCCAG |
| | | | ACAGGTTCAGTGGCAGTGGGTCTGGGACAGACTTCACTCTCACCA |
| | | | TCAGCAGACTGGAGCCTGAAGATTTTGCAGTGTATTACTGTCAGCA |
| | | | TTATGGTAACTCACCGCTCACTTTCGGCGGAGGGACCAAGGTGGA |
| | | | GATCAAACGTACGGTAGCTGCCCCTTCAGTTTTTATCTTTCCGCCG |
| | | | TCTGACGAGCAGTTAAAATCCGGGACCGCTTCTGTAGTTTGCCTGC |
| | | | TGAATAATTTTTATCCGCGTGAGGCTAAAGTACAATGGAAAGTCG |
| | | | ACAATGCTTTGCAGTCGGGAAATTCACAGGAAAGTGTTACGGAGC |
| | | | AGGATTCTAAAGATTCCACATATTCACTCAGCTCCACCCTTACACT |
| | | | GAGCAAAGCCGACTATGAAAAACATAAAGTTTACGCATGTGAGGT |
| | | | GACGCACCAAGGATTATCCAGTCCGGTCACAAAATCGTTTAACCG |
| | | | CGGTGAGTGT |
| 524 | ATX-P-608 | LC Kappa- | GAAATAGTGATGACGCAGTCTCCAGCCACCCTGTCTGTGTCTCCAG |
| | | nucleotide | GGGAAAGAGCCACCCTCTCCTGCAGGGCCAGTCAGAGTGTTAGCA |
| | | | GCAACTTAGCCTGGTACCAGCAGAAACCTGGCCAGGCTCCCAGGC |
| | | | TCCTCATCTATGGTGCATCCACCAGGGCCACTGGTATCCCAGTCAG |
| | | | GTTCAGTGGCAGTGGGTCTGGGACACAGTTCACTCTCACCATCAGC |
| | | | AGCCTGCAGTCTGAAGATTTTGCAGTTTATTATTGTCAGCAATATG |
| | | | ATAACTGGCGGACGTTCGGCCAAGGGACACGACTGGAGATTAAAC |
| | | | GTACGGTAGCTGCCCCTTCAGTTTTTATCTTTCCGCCGTCTGACGA |
| | | | GCAGTTAAAATCCGGGACCGCTTCTGTAGTTTGCCTGCTGAATAAT |
| | | | TTTTATCCGCGTGAGGCTAAAGTACAATGGAAAGTCGACAATGCT |
| | | | TTGCAGTCGGGAAATTCACAGGAAAGTGTTACGGAGCAGGATTCT |
| | | | AAAGATTCCACATATTCACTCAGCTCCACCCTTACACTGAGCAAAG |
| | | | CCGACTATGAAAAACATAAAGTTTACGCATGTGAGGTGACGCACC |
| | | | AAGGATTATCCAGTCCGGTCACAAAATCGTTTAACCGCGGTGAGT |
| | | | GT |
| 525 | ATX-P-609 | LC Kappa- | GAAATAGTGATGACGCAGTCTCCAGCCACCCTGTCTGTGTCTCCAG |
| | | nucleotide | GGGAAAGAGCCACCCTCTCCTGCAGGGCCAGTCAGAGTGTTAGCA |
| | | | GCAACTTAGCCTGGTACCAGCAGAAACCTGGCCAGGCTCCCAGGC |
| | | | TCCTCATCTATGGTGCATCCACCAGGGCCACTGGTATCCCAGTCAG |
| | | | GTTCAGTGGCAGTGGGTCTGGGACACAGTTCACTCTCACCATCAGC |
| | | | AGCCTGCAGTCTGAAGATTTTGCAGTTTATTATTGTCAGCAATATG |
| | | | ATAACTGGCGGACGTTCGGCCAAGGGACCAAGCTGGAAATCAAAC |
| | | | GTACGGTAGCTGCCCCTTCAGTTTTTATCTTTCCGCCGTCTGACGA |
| | | | GCAGTTAAAATCCGGGACCGCTTCTGTAGTTTGCCTGCTGAATAAT |
| | | | TTTTATCCGCGTGAGGCTAAAGTACAATGGAAAGTCGACAATGCT |
| | | | TTGCAGTCGGGAAATTCACAGGAAAGTGTTACGGAGCAGGATTCT |
| | | | AAAGATTCCACATATTCACTCAGCTCCACCCTTACACTGAGCAAAG |
| | | | CCGACTATGAAAAACATAAAGTTTACGCATGTGAGGTGACGCACC |
| | | | AAGGATTATCCAGTCCGGTCACAAAATCGTTTAACCGCGGTGAGT |
| | | | GT |
| 526 | ATX-P-563 | LC Kappa- | GACATCCAGATGACCCAGTCTCCATCTTCCGTGTCTGCATCTGTCG |
| | | nucleotide | GAGACAGAGTCACCATCACTTGTCGGGCGAGTCAGAGTTTTGCCG |
| | | | GCTGGTTAGCCTGGTATCAGCAGAAACCAGGGAAAGCCCCTAAGC |
| | | | TCCTGATATATGCTGCATCCACTTTGCAAAGTGGGGTCCCATCAAG |
| | | | GTTCAGCGGCAGTGGATCTGGGACAGATTTCACTCTCACCATCAGC |
| | | | AGCCTGCAGCCTGAAGATTTTGCAACTTACTATTGTCAACAGGCTA |
| | | | ACAGTTTCCCTCTCACTTTCGGCGGAGGGACCAAGGTGGAGATCA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 527 | ATX-P-570 | LC Kappa- | GACATCGTGATGACCCAGTCTCCAGACTCCCTGGCTGTGTCTCTGG |
| | | nucleotide | GCGAGAGGGCCACCATCAACTGCAAGTCCAGTCAGAGTGTTTTAT |
| | | | ACAGCTCCAACAATAAGAATTACTTAGCTTGGTACCACCAGAAAC |
| | | | CAGGACAGCCTCCTAAGCTGCTCATTTACTGGGCATCTACCCGGGA |
| | | | ATCCGGGGTCCCTGACCGATTCAGTGGCAGCGGGTCTGGGACAGA |
| | | | TTTCACTCTCACCATCAGCAGCCTGCAGGCTGAAGATGTGGCAGTT |
| | | | TATTACTGTCAGCAATACTATAGTAGTCCTCGGACGTTCGGCCAAG |
| | | | GGACCAAGGTGGAAATCAAACGTACGGTAGCTGCCCCTTCAGTTT |
| | | | TTATCTTTCCGCCGTCTGACGAGCAGTTAAAATCCGGGACCGCTTC |
| | | | TGTAGTTTGCCTGCTGAATAATTTTTATCCGCGTGAGGCTAAAGTA |
| | | | CAATGGAAAGTCGACAATGCTTTGCAGTCGGGAAATTCACAGGAA |
| | | | AGTGTTACGGAGCAGGATTCTAAAGATTCCACATATTCACTCAGCT |
| | | | CCACCCTTACACTGAGCAAAGCCGACTATGAAAAACATAAAGTTT |
| | | | ACGCATGTGAGGTGACGCACCAAGGATTATCCAGTCCGGTCACAA |
| | | | AATCGTTTAACCGCGGTGAGTGT |
| 528 | ATX-P-560 | HC IgG1 Fc | QVQLQQSGPGLVKPSQTLSLTCAISGDSVSSNTAAWNWIRQSPSRGLE |
| | | | WLGRTYYRSKWYNDYAVSVKSRITINPDTSKNQFSLQLSSVTPQDTAI |
| | | | YYCASDNWNNGGPGFDPWGQGTLVTVSSASTKGPSVFPLAPSSKSTS |
| | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCPA |
| | | | PELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 529 | ATX-P-561 | HC IgG1 Fc | QVQLQQSGPGLVKPSQTLSLTCAISGDSVSSNSAAWNWIRQSPSRGLE |
| | | | WLGRTYYRSKWYNGYAVSMKSRITINPDTSKNQFSLQLNSVTPEDTA |
| | | | MYYCAREGVGATTGFDYWGQGTLVTVSSASTKGPSVFPLAPSSKSTS |
| | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCPA |
| | | | PELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 530 | ATX-P-562 | HC IgG1 Fc | QVQLQQSGPGLVKSSQTLSLTCAISGDTVSSNSAAWNWIRQSPSRGLE |
| | | | WLGRTFYRSRWSAAYAASVSSRITINPDTSKNQFSLQLTSVTPADTAV |
| | | | YYCAREGVGSSTGFDYWGQGTLVTVSSASTKGPSVFPLAPSSKSTSG |
| | | | GTAALGCLVKDYFPEPVTVSWNSGALTSGVHTFPAVLQSSGLYSLSS |
| | | | VVTVPSSSLGTQTYICNVNHKPSNTKVDKRVEPKSCDKTHTCPPCPAP |
| | | | ELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 531 | ATX-P-566 | HC IgG1 Fc | QVQLQQSGPGLVKPSQTLSLTCAISGDSVSSNSAAWNWIRQSPSRGLE |
| | | | WLGRTYYRSKWFNNYSVSVKSRIAINPDTSKNQFSLQLNSVTPEDTA |
| | | | VYYCARDLGELLDWYFDLWGRGTLVTVSAASTKGPSVFPLAPSSKST |
| | | | SGGTAALGCLVKDYFPEPVTVSWNSGALTSGVHTFPAVLQSSGLYSL |
| | | | SSVVTVPSSSLGTQTYICNVNHKPSNTKVDKRVEPKSCDKTHTCPPCP |
| | | | APELLGGPSVFLFPPKPKDTLMISRTPEVTCVVVDVSHEDPEVKFNWY |
| | | | VDGVEVHNAKTKPREEQYASTYRVVSVLTVLHQDWLNGKEYKCKV |
| | | | SNKALPAPIEKTISKAKGQPREPQVYTLPPSREEMTKNQVSLTCLVKG |
| | | | FYPSDIAVEWESNGQPENNYKTTPPVLDSDGSFFLYSKLTVDKSRWQ |
| | | | QGNVFSCSVMHEALHNHYTQKSLSLSPG |
| 532 | ATX-P-572 | HC IgG1 Fc | QVQLQQSGPGLVKPSQTLSLTCAISGDSVSSNSAAWNWIRQSPSRGLE |
| | | | WLGMTYYRSKWYSVFAVSVKSRITINTDTSKNQFSLQLNSVTPEDTA |
| | | | VYYCAREGGIVGATPFDYWGQGTLVTVSSASTKGPSVFPLAPSSKSTS |
| | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCPA |
| | | | PELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 533 | ATX-P-573 | HC IgG1 Fc | QVQLQQSGPGLVKPSQTLSLTCAISGDSVSSNSAAWNWIRQSPSRGLE |
| | | | WLGRTYYRSKLYSDYAVSVKSRITINPDTSRNQFSLQLNSVTPEDTAV |
| | | | YYCAREGGYTYGYDYWGQGTLVTVSSASTKGPSVFPLAPSSKSTSGG |
| | | | TAALGCLVKDYFPEPVTVSWNSGALTSGVHTFPAVLQSSGLYSLSSV |
| | | | VTVPSSSLGTQTYICNVNHKPSNTKVDKRVEPKSCDKTHTCPPCPAPE |
| | | | LLGGPSVFLFPPKPKDTLMISRTPEVTCVVVDVSHEDPEVKFNWYVD |
| | | | GVEVHNAKTKPREEQYASTYRVVSVLTVLHQDWLNGKEYKCKVSN |
| | | | KALPAPIEKTISKAKGQPREPQVYTLPPSREEMTKNQVSLTCLVKGFY |
| | | | PSDIAVEWESNGQPENNYKTTPPVLDSDGSFFLYSKLTVDKSRWQQG |
| | | | NVFSCSVMHEALHNHYTQKSLSLSPG |
| 534 | ATX-P-581 | HC IgG1 Fc | QVQLQQSGPGLVKPSQTLSLTCAISGDSVSSNNAAWNWIRQSPSRGLE |
| | | | WLGRTYYRSKWYNAYAVSVKSRIIINPDTSRNQFSLQLNSVTPEDTA |
| | | | VYYCAREGGYIYGLDQWGQGTLVTVSSASTKGPSVFPLAPSSKSTSG |
| | | | GTAALGCLVKDYFPEPVTVSWNSGALTSGVHTFPAVLQSSGLYSLSS |
| | | | VVTVPSSSLGTQTYICNVNHKPSNTKVDKRVEPKSCDKTHTCPPCPAP |
| | | | ELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 535 | ATX-P-587 | HC IgG1 Fc | QVQLQQSGPGLVKPSQTLSLTCAISGDSVSSNSAAWNWIRQSPSRGLE |
| | | | WLGKTYYRSKWYNYYALSVKSRITINPDTSKNQFSLHLNSVTPEDTA |
| | | | VYYCARDGDYYLPSPFDLWGHGTMVTVSSASTKGPSVFPLAPSSKST |
| | | | SGGTAALGCLVKDYFPEPVTVSWNSGALTSGVHTFPAVLQSSGLYSL |
| | | | SSVVTVPSSSLGTQTYICNVNHKPSNTKVDKRVEPKSCDKTHTCPPCP |
| | | | APELLGGPSVFLFPPKPKDTLMISRTPEVTCVVVDVSHEDPEVKFNWY |
| | | | VDGVEVHNAKTKPREEQYASTYRVVSVLTVLHQDWLNGKEYKCKV |
| | | | SNKALPAPIEKTISKAKGQPREPQVYTLPPSREEMTKNQVSLTCLVKG |
| | | | FYPSDIAVEWESNGQPENNYKTTPPVLDSDGSFFLYSKLTVDKSRWQ |
| | | | QGNVFSCSVMHEALHNHYTQKSLSLSPG |
| 536 | ATX-P-592 | HC IgG1 Fc | QVQLQQSGPGLVKPSQTLSLTCAISGDSVSSNSAAWNWIRQSPSRGLE |
| | | | WLGRTYYRSKWYSDYPLSVKSRITINPDTSKNQFSLQLNSVTPEDTAV |
| | | | YYCARDSGIVETTPFDYWGQGTLVTVSSASTKGPSVFPLAPSSKSTSG |
| | | | GTAALGCLVKDYFPEPVTVSWNSGALTSGVHTFPAVLQSSGLYSLSS |
| | | | VVTVPSSSLGTQTYICNVNHKPSNTKVDKRVEPKSCDKTHTCPPCPAP |
| | | | ELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 537 | ATX-P-594 | HC IgG1 Fc | QVQLQQSGPGLVKPSQTLSLTCAISGDSVSSNSASWNWIRQSPSRGLE |
| | | | WLGTTYYRSKWFNVYAVSVKSRITINPDTSKNQFSLHLNSVTPEDTAI |
| | | | YFCTRDRGDLLHWYFDLWGRGTLVTVSSASTKGPSVFPLAPSSKSTS |
| | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCPA |
| | | | PELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 538 | ATX-P-595 | HC IgG1 Fc | QVQLQQSGPGLVKPSQTLSLTCAISGDSVSSNSAAWNWIRQSPSRGLE |
| | | | WLGRTYYRSKWFNDYALSVKSRITINPDTSKNQFSLHLNSVTPEDTA |
| | | | VYYCAREPGIVAPGPFDYWGQGTLVTVSSASTKGPSVFPLAPSSKSTS |
| | | | GGTAALGCLVKDYFPEPVTVSWNSGALTSGVHTFPAVLQSSGLYSLS |
| | | | SVVTVPSSSLGTQTYICNVNHKPSNTKVDKRVEPKSCDKTHTCPPCPA |
| | | | PELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 539 | ATX-P-604 | HC IgG1 Fc | QVQLQQSGPGLVKPTQTLSLTCAISGDSVSSNSAAWNWIRQSPSRGLE |
| | | | WLGRTYYRSKWFNGYAVSVKSRITINPDASKNQFSLHLKSVTDEDTA |
| | | | VYYCATDRGELLHWYFDLWGRGTLVTVSSASTKGPSVFPLAPSSKST |
| | | | SGGTAALGCLVKDYFPEPVTVSWNSGALTSGVHTFPAVLQSSGLYSL |
| | | | SSVVTVPSSSLGTQTYICNVNHKPSNTKVDKRVEPKSCDKTHTCPPCP |
| | | | APELLGGPSVFLFPPKPKDTLMISRTPEVTCVVVDVSHEDPEVKFNWY |
| | | | VDGVEVHNAKTKPREEQYASTYRVVSVLTVLHQDWLNGKEYKCKV |
| | | | SNKALPAPIEKTISKAKGQPREPQVYTLPPSREEMIKNQVSLTCLVKG |
| | | | FYPSDIAVEWESNGQPENNYKTTPPVLDSDGSFFLYSKLTVDKSRWQ |
| | | | QGNVFSCSVMHEALHNHYTQKSLSLSPG |
| 540 | ATX-P-610 | HC IgG1 Fc | QVQLQQSGPGLVKSSQTLSITCAISGDSVSSNSAAWNWIRQSPSRGLE |
| | | | WLGRTYYRSKWYNDYALSVKSRIIIKSDTSKNQFSLQLNSVTPEDTAV |
| | | | YYCAREPGIAAPGPFDYWGQGSLVTVSSASTKGPSVFPLAPSSKSTSG |
| | | | GTAALGCLVKDYFPEPVTVSWNSGALTSGVHTFPAVLQSSGLYSLSS |
| | | | VVTVPSSSLGTQTYICNVNHKPSNTKVDKRVEPKSCDKTHTCPPCPAP |
| | | | ELLGGPSVFLFPPKPKDTLMISRTPEVTCVVVDVSHEDPEVKFNWYV |
| | | | DGVEVHNAKTKPREEQYASTYRVVSVLTVLHQDWLNGKEYKCKVS |
| | | | NKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQ |
| | | | GNVFSCSVMHEALHNHYTQKSLSLSPG |
| 541 | ATX-P-560 | HC IgG1 Fc- | CAGGTACAGCTGCAGCAGTCAGGTCCAGGACTGGTGAAGCCCTCG |
| | | nucleotide | CAGACCCTCTCACTCACCTGTGCCATTTCCGGGGACAGTGTCTCTA |
| | | | GCAACACTGCTGCTTGGAACTGGATCAGGCAGTCCCCATCGAGAG |
| | | | GCCTTGAGTGGCTGGGAAGGACATACTACAGGTCCAAGTGGTATA |
| | | | ATGATTATGCAGTATCTGTGAAAAGTCGAATAACCATCAACCCAG |
| | | | ACACATCCAAGAACCAGTTCTCCCTGCAGTTGAGCTCTGTGACTCC |
| | | | CCAGGACACGGCTATATATTACTGTGCGAGCGATAACTGGAACAA |
| | | | CGGAGGCCCTGGGTTCGACCCCTGGGGCCAGGGAACCCTGGTCAC |
| | | | CGTCTCCTCAGCTAGCACTAAAGGGCCTTCTGTATTTCCCTTGGCC |
| | | | CCGTCCAGCAAATCGACCTCGGGAGGGACAGCCGCCCTGGGTTGC |
| | | | CTTGTGAAAGATTATTTCCCTGAGCCAGTTACCGTAAGTTGGAACA |
| | | | GTGGGGCGCTGACAAGTGGTGTGCACACGTTTCCTGCCGTCCTGCA |
| | | | ATCATCGGGCTTGTATAGCCTCAGCTCTGTGGTCACTGTCCCAAGT |
| | | | TCATCGCTGGGCACTCAGACGTATATTTGCAATGTGAACCACAAA |
| | | | CCTTCAAATACAAAAGTGGATAAACGCGTAGAACCGAAATCGTGT |
| | | | GATAAAACTCACACATGCCCGCCATGCCCGGCACCTGAACTGCTT |
| | | | GGTGGTCCCAGCGTGTTCCTGTTCCCGCCGAAGCCTAAAGATACTC |
| | | | TAATGATCAGCCGTACGCCAGAGGTGACATGTGTCGTGGTTGACG |
| | | | TGTCCCACGAAGATCCCGAAGTTAAGTTCAATTGGTATGTTGATGG |
| | | | TGTAGAGGTACACAATGCTAAGACTAAACCTCGCGAGGAGCAGTA |
| | | | CGCCTCGACCTATCGTGTCGTGAGCGTTCTGACCGTCCTTCACCAA |
| | | | GATTGGCTTAACGGCAAAGAATATAAGTGCAAGGTAAGCAATAAA |
| | | | GCACTTCCGGCCCCAATCGAGAAAACCATTTCCAAGGCCAAAGGT |
| | | | CAACCAAGAGAACCCCAGGTGTATACTCTTCCGCCTTCTCGTGAGG |
| | | | AAATGACTAAAAATCAAGTATCCCTTACGTGTCTGGTTAAAGGTTT |
| | | | TTATCCTAGCGATATTGCTGTTGAATGGGAATCGAACGGTCAGCCG |
| | | | GAGAATAATTATAAAACAACGCCACCCGTCCTGGATAGCGACGGC |
| | | | TCATTTTTTCTGTATAGCAAACTGACTGTAGATAAATCACGGTGGC |
| | | | AGCAGGGCAATGTATTCAGTTGCTCCGTTATGCATGAAGCGTTACA |
| | | | TAATCACTACACGCAGAAATCTCTTAGTCTTTCACCCGGT |
| 542 | ATX-P-561 | HC IgG1 Fc- | CAGGTACAGCTGCAGCAGTCAGGTCCAGGACTGGTGAAGCCCTCG |
| | | nucleotide | CAGACCCTCTCACTCACCTGTGCCATCTCCGGGGACAGTGTCTCTA |
| | | | GCAACAGTGCTGCTTGGAACTGGATCAGGCAGTCCCCATCGAGAG |
| | | | GCCTTGAGTGGCTGGGAAGGACATACTACAGGTCCAAGTGGTATA |
| | | | ATGGTTATGCAGTCTCTATGAAAAGTCGAATAACCATCAACCCAG |
| | | | ACACATCCAAGAACCAGTTCTCCCTGCAGCTGAACTCTGTGACTCC |
| | | | CGAGGACACGGCTATGTATTATTGTGCAAGAGAGGGAGTGGGAGC |
| | | | TACTACAGGCTTTGACTACTGGGGCCAGGGAACCCTGGTCACCGT |
| | | | CTCCTCAGCTAGCACTAAAGGGCCTTCTGTATTTCCCTTGGCCCCG |
| | | | TCCAGCAAATCGACCTCGGGAGGGACAGCCGCCCTGGGTTGCCTT |
| | | | GTGAAAGATTATTTCCCTGAGCCAGTTACCGTAAGTTGGAACAGT |
| | | | GGGGCGCTGACAAGTGGTGTGCACACGTTTCCTGCCGTCCTGCAAT |
| | | | CATCGGGCTTGTATAGCCTCAGCTCTGTGGTCACTGTCCCAAGTTC |
| | | | ATCGCTGGGCACTCAGACGTATATTTGCAATGTGAACCACAAACC |
| | | | TTCAAATACAAAAGTGGATAAACGCGTAGAACCGAAATCGTGTGA |
| | | | TAAAACTCACACATGCCCGCCATGCCCGGCACCTGAACTGCTTGGT |
| | | | GGTCCCAGCGTGTTCCTGTTCCCGCCGAAGCCTAAAGATACTCTAA |
| | | | TGATCAGCCGTACGCCAGAGGTGACATGTGTCGTGGTTGACGTGT |
| | | | CCCACGAAGATCCCGAAGTTAAGTTCAATTGGTATGTTGATGGTGT |
| | | | AGAGGTACACAATGCTAAGACTAAACCTCGCGAGGAGCAGTACGC |
| | | | CTCGACCTATCGTGTCGTGAGCGTTCTGACCGTCCTTCACCAAGAT |
| | | | TGGCTTAACGGCAAAGAATATAAGTGCAAGGTAAGCAATAAAGCA |
| | | | CTTCCGGCCCCAATCGAGAAAACCATTTCCAAGGCCAAAGGTCAA |
| | | | CCAAGAGAACCCCAGGTGTATACTCTTCCGCCTTCTCGTGAGGAA |
| | | | ATGACTAAAAATCAAGTATCCCTTACGTGTCTGGTTAAAGGTTTTT |
| | | | ATCCTAGCGATATTGCTGTTGAATGGGAATCGAACGGTCAGCCGG |
| | | | AGAATAATTATAAAACAACGCCACCCGTCCTGGATAGCGACGGCT |
| | | | CATTTTTTCTGTATAGCAAACTGACTGTAGATAAATCACGGTGGCA |
| | | | GCAGGGCAATGTATTCAGTTGCTCCGTTATGCATGAAGCGTTACAT |
| | | | AATCACTACACGCAGAAATCTCTTAGTCTTTCACCCGGT |
| 543 | ATX-P-562 | HC IgG1 Fc- | CAGGTACAGCTGCAGCAGTCAGGTCCAGGACTGGTGAAGTCCTCG |
| | | nucleotide | CAGACCCTCTCACTCACCTGTGCCATCTCCGGGGACACTGTCTCTA |
| | | | GTAATAGTGCTGCTTGGAACTGGATCAGGCAGTCCCCATCGAGAG |
| | | | GCCTTGAGTGGCTGGGAAGGACATTCTATAGGTCCAGGTGGTCTG |
| | | | CTGCTTATGCAGCGTCTGTGAGTAGTCGAATAACCATCAACCCAG |
| | | | ACACATCCAAGAACCAGTTCTCCCTGCAGCTGACCTCTGTGACTCC |
| | | | CGCGGACACGGCTGTGTATTACTGTGCAAGAGAGGGAGTAGGATC |
| | | | TTCTACAGGCTTTGACTACTGGGGCCAGGGAACCCTGGTCACCGTC |
| | | | TCCTCAGCTAGCACTAAAGGGCCTTCTGTATTTCCCTTGGCCCCGT |
| | | | CCAGCAAATCGACCTCGGGAGGGACAGCCGCCCTGGGTTGCCTTG |
| | | | TGAAAGATTATTTCCCTGAGCCAGTTACCGTAAGTTGGAACAGTG |
| | | | GGGCGCTGACAAGTGGTGTGCACACGTTTCCTGCCGTCCTGCAATC |
| | | | ATCGGGCTTGTATAGCCTCAGCTCTGTGGTCACTGTCCCAAGTTCA |
| | | | TCGCTGGGCACTCAGACGTATATTTGCAATGTGAACCACAAACCTT |
| | | | CAAATACAAAAGTGGATAAACGCGTAGAACCGAAATCGTGTGATA |
| | | | AAACTCACACATGCCCGCCATGCCCGGCACCTGAACTGCTTGGTG |
| | | | GTCCCAGCGTGTTCCTGTTCCCGCCGAAGCCTAAAGATACTCTAAT |
| | | | GATCAGCCGTACGCCAGAGGTGACATGTGTCGTGGTTGACGTGTC |
| | | | CCACGAAGATCCCGAAGTTAAGTTCAATTGGTATGTTGATGGTGTA |
| | | | GAGGTACACAATGCTAAGACTAAACCTCGCGAGGAGCAGTACGCC |
| | | | TCGACCTATCGTGTCGTGAGCGTTCTGACCGTCCTTCACCAAGATT |
| | | | GGCTTAACGGCAAAGAATATAAGTGCAAGGTAAGCAATAAAGCA |
| | | | CTTCCGGCCCCAATCGAGAAAACCATTTCCAAGGCCAAAGGTCAA |
| | | | CCAAGAGAACCCCAGGTGTATACTCTTCCGCCTTCTCGTGAGGAA |
| | | | ATGACTAAAAATCAAGTATCCCTTACGTGTCTGGTTAAAGGTTTTT |
| | | | ATCCTAGCGATATTGCTGTTGAATGGGAATCGAACGGTCAGCCGG |
| | | | AGAATAATTATAAAACAACGCCACCCGTCCTGGATAGCGACGGCT |
| | | | CATTTTTTCTGTATAGCAAACTGACTGTAGATAAATCACGGTGGCA |
| | | | GCAGGGCAATGTATTCAGTTGCTCCGTTATGCATGAAGCGTTACAT |
| | | | AATCACTACACGCAGAAATCTCTTAGTCTTTCACCCGGT |
| 544 | ATX-P-566 | HC IgG1 Fc- | CAGGTACAGCTGCAGCAGTCAGGTCCAGGACTGGTGAAGCCCTCG |
| | | nucleotide | CAGACCCTCTCACTCACCTGTGCCATCTCCGGGGACAGTGTCTCTA |
| | | | GCAACAGTGCTGCTTGGAACTGGATCAGGCAGTCCCCATCGAGAG |
| | | | GCCTTGAGTGGCTGGGAAGGACATACTACAGGTCCAAGTGGTTTA |
| | | | ATAATTATTCAGTTTCTGTGAAAAGTCGAATAGCCATTAACCCAGA |
| | | | CACATCCAAGAACCAGTTCTCCCTTCAACTGAACTCTGTGACTCCC |
| | | | GAGGACACGGCTGTGTATTACTGTGCAAGAGATCTGGGGGAGTTA |
| | | | CTTGACTGGTACTTCGATCTCTGGGGCCGTGGCACCCTGGTCACTG |
| | | | TCTCCGCAGCTAGCACTAAAGGGCCTTCTGTATTTCCCTTGGCCCC |
| | | | GTCCAGCAAATCGACCTCGGGAGGGACAGCCGCCCTGGGTTGCCT |
| | | | TGTGAAAGATTATTTCCCTGAGCCAGTTACCGTAAGTTGGAACAGT |
| | | | GGGGCGCTGACAAGTGGTGTGCACACGTTTCCTGCCGTCCTGCAAT |
| | | | CATCGGGCTTGTATAGCCTCAGCTCTGTGGTCACTGTCCCAAGTTC |
| | | | ATCGCTGGGCACTCAGACGTATATTTGCAATGTGAACCACAAACC |
| | | | TTCAAATACAAAAGTGGATAAACGCGTAGAACCGAAATCGTGTGA |
| | | | TAAAACTCACACATGCCCGCCATGCCCGGCACCTGAACTGCTTGGT |
| | | | GGTCCCAGCGTGTTCCTGTTCCCGCCGAAGCCTAAAGATACTCTAA |
| | | | TGATCAGCCGTACGCCAGAGGTGACATGTGTCGTGGTTGACGTGT |
| | | | CCCACGAAGATCCCGAAGTTAAGTTCAATTGGTATGTTGATGGTGT |
| | | | AGAGGTACACAATGCTAAGACTAAACCTCGCGAGGAGCAGTACGC |
| | | | CTCGACCTATCGTGTCGTGAGCGTTCTGACCGTCCTTCACCAAGAT |
| | | | TGGCTTAACGGCAAAGAATATAAGTGCAAGGTAAGCAATAAAGCA |
| | | | CTTCCGGCCCCAATCGAGAAAACCATTTCCAAGGCCAAAGGTCAA |
| | | | CCAAGAGAACCCCAGGTGTATACTCTTCCGCCTTCTCGTGAGGAA |
| | | | ATGACTAAAAATCAAGTATCCCTTACGTGTCTGGTTAAAGGTTTTT |
| | | | ATCCTAGCGATATTGCTGTTGAATGGGAATCGAACGGTCAGCCGG |
| | | | AGAATAATTATAAAACAACGCCACCCGTCCTGGATAGCGACGGCT |
| | | | CATTTTTTCTGTATAGCAAACTGACTGTAGATAAATCACGGTGGCA |
| | | | GCAGGGCAATGTATTCAGTTGCTCCGTTATGCATGAAGCGTTACAT |
| | | | AATCACTACACGCAGAAATCTCTTAGTCTTTCACCCGGT |
| 545 | ATX-P-572 | HC IgG1 Fc- | CAGGTACAGCTGCAGCAGTCAGGTCCAGGACTGGTGAAGCCCTCG |
| | | nucleotide | CAGACCCTCTCACTCACCTGTGCCATCTCCGGGGACAGTGTCTCTA |
| | | | GCAACAGTGCTGCTTGGAACTGGATCAGGCAGTCCCCATCGAGAG |
| | | | GCCTTGAGTGGCTGGGAATGACATACTACAGGTCCAAGTGGTATA |
| | | | GTGTTTTTGCAGTATCTGTGAAAAGTCGAATAACCATCAATACAGA |
| | | | CACATCCAAGAACCAGTTCTCCCTGCAGCTGAACTCTGTGACTCCC |
| | | | GAGGACACGGCTGTGTATTACTGTGCAAGAGAGGGGGGTATAGTG |
| | | | GGAGCCACCCCCTTTGACTACTGGGGCCAGGGAACCCTGGTCACC |
| | | | GTCTCTTCAGCTAGCACTAAAGGGCCTTCTGTATTTCCCTTGGCCC |
| | | | CGTCCAGCAAATCGACCTCGGGAGGGACAGCCGCCCTGGGTTGCC |
| | | | TTGTGAAAGATTATTTCCCTGAGCCAGTTACCGTAAGTTGGAACAG |
| | | | TGGGGCGCTGACAAGTGGTGTGCACACGTTTCCTGCCGTCCTGCAA |
| | | | TCATCGGGCTTGTATAGCCTCAGCTCTGTGGTCACTGTCCCAAGTT |
| | | | CATCGCTGGGCACTCAGACGTATATTTGCAATGTGAACCACAAAC |
| | | | CTTCAAATACAAAAGTGGATAAACGCGTAGAACCGAAATCGTGTG |
| | | | ATAAAACTCACACATGCCCGCCATGCCCGGCACCTGAACTGCTTG |
| | | | GTGGTCCCAGCGTGTTCCTGTTCCCGCCGAAGCCTAAAGATACTCT |
| | | | AATGATCAGCCGTACGCCAGAGGTGACATGTGTCGTGGTTGACGT |
| | | | GTCCCACGAAGATCCCGAAGTTAAGTTCAATTGGTATGTTGATGGT |
| | | | GTAGAGGTACACAATGCTAAGACTAAACCTCGCGAGGAGCAGTAC |
| | | | GCCTCGACCTATCGTGTCGTGAGCGTTCTGACCGTCCTTCACCAAG |
| | | | ATTGGCTTAACGGCAAAGAATATAAGTGCAAGGTAAGCAATAAAG |
| | | | CACTTCCGGCCCCAATCGAGAAAACCATTTCCAAGGCCAAAGGTC |
| | | | AACCAAGAGAACCCCAGGTGTATACTCTTCCGCCTTCTCGTGAGG |
| | | | AAATGACTAAAAATCAAGTATCCCTTACGTGTCTGGTTAAAGGTTT |
| | | | TTATCCTAGCGATATTGCTGTTGAATGGGAATCGAACGGTCAGCCG |
| | | | GAGAATAATTATAAAACAACGCCACCCGTCCTGGATAGCGACGGC |
| | | | TCATTTTTTCTGTATAGCAAACTGACTGTAGATAAATCACGGTGGC |
| | | | AGCAGGGCAATGTATTCAGTTGCTCCGTTATGCATGAAGCGTTACA |
| | | | TAATCACTACACGCAGAAATCTCTTAGTCTTTCACCCGGT |
| 546 | ATX-P-573 | HC IgG1 Fc- | CAGGTACAGCTGCAGCAGTCAGGTCCAGGACTGGTGAAGCCCTCG |
| | | nucleotide | CAGACCCTCTCACTCACCTGTGCCATCTCCGGGGACAGTGTCTCTA |
| | | | GCAACAGTGCTGCTTGGAACTGGATCAGGCAGTCCCCATCGAGAG |
| | | | GCCTTGAGTGGCTGGGCAGGACATACTACAGGTCCAAGTTGTATA |
| | | | GTGATTATGCAGTATCTGTGAAAAGTCGAATAACCATCAACCCAG |
| | | | ACACATCCAGGAACCAGTTCTCCCTGCAGCTGAACTCTGTGACTCC |
| | | | CGAGGACACGGCTGTGTATTACTGTGCAAGAGAGGGGGGATACAC |
| | | | CTATGGTTATGACTACTGGGGCCAGGGAACCCTGGTCACCGTCTCC |
| | | | TCAGCTAGCACTAAAGGGCCTTCTGTATTTCCCTTGGCCCCGTCCA |
| | | | GCAAATCGACCTCGGGAGGGACAGCCGCCCTGGGTTGCCTTGTGA |
| | | | AAGATTATTTCCCTGAGCCAGTTACCGTAAGTTGGAACAGTGGGG |
| | | | CGCTGACAAGTGGTGTGCACACGTTTCCTGCCGTCCTGCAATCATC |
| | | | GGGCTTGTATAGCCTCAGCTCTGTGGTCACTGTCCCAAGTTCATCG |
| | | | CTGGGCACTCAGACGTATATTTGCAATGTGAACCACAAACCTTCA |
| | | | AATACAAAAGTGGATAAACGCGTAGAACCGAAATCGTGTGATAAA |
| | | | ACTCACACATGCCCGCCATGCCCGGCACCTGAACTGCTTGGTGGTC |
| | | | CCAGCGTGTTCCTGTTCCCGCCGAAGCCTAAAGATACTCTAATGAT |
| | | | CAGCCGTACGCCAGAGGTGACATGTGTCGTGGTTGACGTGTCCCA |
| | | | CGAAGATCCCGAAGTTAAGTTCAATTGGTATGTTGATGGTGTAGA |
| | | | GGTACACAATGCTAAGACTAAACCTCGCGAGGAGCAGTACGCCTC |
| | | | GACCTATCGTGTCGTGAGCGTTCTGACCGTCCTTCACCAAGATTGG |
| | | | CTTAACGGCAAAGAATATAAGTGCAAGGTAAGCAATAAAGCACTT |
| | | | CCGGCCCCAATCGAGAAAACCATTTCCAAGGCCAAAGGTCAACCA |
| | | | AGAGAACCCCAGGTGTATACTCTTCCGCCTTCTCGTGAGGAAATG |
| | | | ACTAAAAATCAAGTATCCCTTACGTGTCTGGTTAAAGGTTTTTATC |
| | | | CTAGCGATATTGCTGTTGAATGGGAATCGAACGGTCAGCCGGAGA |
| | | | ATAATTATAAAACAACGCCACCCGTCCTGGATAGCGACGGCTCAT |
| | | | TTTTTCTGTATAGCAAACTGACTGTAGATAAATCACGGTGGCAGCA |
| | | | GGGCAATGTATTCAGTTGCTCCGTTATGCATGAAGCGTTACATAAT |
| | | | CACTACACGCAGAAATCTCTTAGTCTTTCACCCGGT |
| 547 | ATX-P-581 | HC IgG1 Fc- | CAGGTACAGCTGCAGCAGTCAGGTCCAGGACTGGTGAAGCCCTCG |
| | | nucleotide | CAGACCCTCTCACTCACCTGTGCCATCTCCGGGGACAGTGTCTCTA |
| | | | GCAACAATGCTGCTTGGAACTGGATCAGGCAGTCCCCATCGAGAG |
| | | | GCCTTGAGTGGCTGGGAAGGACGTACTACAGGTCCAAGTGGTATA |
| | | | ATGCTTATGCAGTTTCTGTGAAAAGTCGAATTATCATCAATCCAGA |
| | | | CACATCCAGGAACCAGTTCTCCCTGCAACTGAACTCTGTGACTCCC |
| | | | GAGGACACGGCTGTGTATTACTGTGCAAGAGAGGGTGGATACATC |
| | | | TATGGTCTTGACCAATGGGGCCAGGGAACCCTGGTCACCGTCTCCT |
| | | | CAGCTAGCACTAAAGGGCCTTCTGTATTTCCCTTGGCCCCGTCCAG |
| | | | CAAATCGACCTCGGGAGGGACAGCCGCCCTGGGTTGCCTTGTGAA |
| | | | AGATTATTTCCCTGAGCCAGTTACCGTAAGTTGGAACAGTGGGGC |
| | | | GCTGACAAGTGGTGTGCACACGTTTCCTGCCGTCCTGCAATCATCG |
| | | | GGCTTGTATAGCCTCAGCTCTGTGGTCACTGTCCCAAGTTCATCGC |
| | | | TGGGCACTCAGACGTATATTTGCAATGTGAACCACAAACCTTCAA |
| | | | ATACAAAAGTGGATAAACGCGTAGAACCGAAATCGTGTGATAAAA |
| | | | CTCACACATGCCCGCCATGCCCGGCACCTGAACTGCTTGGTGGTCC |
| | | | CAGCGTGTTCCTGTTCCCGCCGAAGCCTAAAGATACTCTAATGATC |
| | | | AGCCGTACGCCAGAGGTGACATGTGTCGTGGTTGACGTGTCCCAC |
| | | | GAAGATCCCGAAGTTAAGTTCAATTGGTATGTTGATGGTGTAGAG |
| | | | GTACACAATGCTAAGACTAAACCTCGCGAGGAGCAGTACGCCTCG |
| | | | ACCTATCGTGTCGTGAGCGTTCTGACCGTCCTTCACCAAGATTGGC |
| | | | TTAACGGCAAAGAATATAAGTGCAAGGTAAGCAATAAAGCACTTC |
| | | | CGGCCCCAATCGAGAAAACCATTTCCAAGGCCAAAGGTCAACCAA |
| | | | GAGAACCCCAGGTGTATACTCTTCCGCCTTCTCGTGAGGAAATGAC |
| | | | TAAAAATCAAGTATCCCTTACGTGTCTGGTTAAAGGTTTTTATCCT |
| | | | AGCGATATTGCTGTTGAATGGGAATCGAACGGTCAGCCGGAGAAT |
| | | | AATTATAAAACAACGCCACCCGTCCTGGATAGCGACGGCTCATTTT |
| | | | TTCTGTATAGCAAACTGACTGTAGATAAATCACGGTGGCAGCAGG |
| | | | GCAATGTATTCAGTTGCTCCGTTATGCATGAAGCGTTACATAATCA |
| | | | CTACACGCAGAAATCTCTTAGTCTTTCACCCGGT |
| 548 | ATX-P-587 | HC IgG1 Fc- | CAGGTGCAGCTGCAGCAGTCGGGTCCAGGACTGGTGAAGCCCTCG |
| | | nucleotide | CAGACCCTCTCACTCACCTGTGCCATCTCCGGGGACAGTGTCTCTA |
| | | | GCAACAGTGCTGCTTGGAACTGGATCAGGCAGTCCCCATCGAGAG |
| | | | GCCTTGAGTGGCTGGGAAAGACATACTACAGGTCCAAGTGGTATA |
| | | | ATTATTATGCACTATCTGTGAAAAGTCGAATAACCATCAACCCAG |
| | | | ACACATCCAAGAACCAGTTCTCCCTGCACCTGAACTCTGTGACTCC |
| | | | CGAGGACACGGCTGTGTATTACTGTGCAAGAGACGGTGACTACTA |
| | | | CCTTCCCTCTCCTTTTGATCTCTGGGGCCACGGGACAATGGTCACC |
| | | | GTCTCCTCAGCTAGCACTAAAGGGCCTTCTGTATTTCCCTTGGCCC |
| | | | CGTCCAGCAAATCGACCTCGGGAGGGACAGCCGCCCTGGGTTGCC |
| | | | TTGTGAAAGATTATTTCCCTGAGCCAGTTACCGTAAGTTGGAACAG |
| | | | TGGGGCGCTGACAAGTGGTGTGCACACGTTTCCTGCCGTCCTGCAA |
| | | | TCATCGGGCTTGTATAGCCTCAGCTCTGTGGTCACTGTCCCAAGTT |
| | | | CATCGCTGGGCACTCAGACGTATATTTGCAATGTGAACCACAAAC |
| | | | CTTCAAATACAAAAGTGGATAAACGCGTAGAACCGAAATCGTGTG |
| | | | ATAAAACTCACACATGCCCGCCATGCCCGGCACCTGAACTGCTTG |
| | | | GTGGTCCCAGCGTGTTCCTGTTCCCGCCGAAGCCTAAAGATACTCT |
| | | | AATGATCAGCCGTACGCCAGAGGTGACATGTGTCGTGGTTGACGT |
| | | | GTCCCACGAAGATCCCGAAGTTAAGTTCAATTGGTATGTTGATGGT |
| | | | GTAGAGGTACACAATGCTAAGACTAAACCTCGCGAGGAGCAGTAC |
| | | | GCCTCGACCTATCGTGTCGTGAGCGTTCTGACCGTCCTTCACCAAG |
| | | | ATTGGCTTAACGGCAAAGAATATAAGTGCAAGGTAAGCAATAAAG |
| | | | CACTTCCGGCCCCAATCGAGAAAACCATTTCCAAGGCCAAAGGTC |
| | | | AACCAAGAGAACCCCAGGTGTATACTCTTCCGCCTTCTCGTGAGG |
| | | | AAATGACTAAAAATCAAGTATCCCTTACGTGTCTGGTTAAAGGTTT |
| | | | TTATCCTAGCGATATTGCTGTTGAATGGGAATCGAACGGTCAGCCG |
| | | | GAGAATAATTATAAAACAACGCCACCCGTCCTGGATAGCGACGGC |
| | | | TCATTTTTTCTGTATAGCAAACTGACTGTAGATAAATCACGGTGGC |
| | | | AGCAGGGCAATGTATTCAGTTGCTCCGTTATGCATGAAGCGTTACA |
| | | | TAATCACTACACGCAGAAATCTCTTAGTCTTTCACCCGGT |
| 549 | ATX-P-592 | HC IgG1 Fc- | CAGGTACAGCTGCAGCAGTCAGGTCCAGGACTGGTGAAGCCCTCG |
| | | nucleotide | CAGACCCTCTCACTCACCTGTGCCATCTCCGGGGACAGTGTCTCTA |
| | | | GCAACAGTGCTGCTTGGAACTGGATCAGGCAGTCCCCATCGAGAG |
| | | | GCCTTGAGTGGCTGGGAAGGACATACTACAGGTCCAAGTGGTATA |
| | | | GTGATTATCCACTATCTGTGAAAAGTCGAATAACCATCAACCCCG |
| | | | ACACATCCAAGAACCAGTTCTCCCTGCAGCTGAACTCTGTGACTCC |
| | | | CGAGGACACGGCTGTGTATTACTGTGCAAGAGACTCCGGTATAGT |
| | | | TGAAACCACGCCCTTTGACTATTGGGGCCAGGGAACCCTGGTCAC |
| | | | CGTCTCCTCAGCTAGCACTAAAGGGCCTTCTGTATTTCCCTTGGCC |
| | | | CCGTCCAGCAAATCGACCTCGGGAGGGACAGCCGCCCTGGGTTGC |
| | | | CTTGTGAAAGATTATTTCCCTGAGCCAGTTACCGTAAGTTGGAACA |
| | | | GTGGGGCGCTGACAAGTGGTGTGCACACGTTTCCTGCCGTCCTGCA |
| | | | ATCATCGGGCTTGTATAGCCTCAGCTCTGTGGTCACTGTCCCAAGT |
| | | | TCATCGCTGGGCACTCAGACGTATATTTGCAATGTGAACCACAAA |
| | | | CCTTCAAATACAAAAGTGGATAAACGCGTAGAACCGAAATCGTGT |
| | | | GATAAAACTCACACATGCCCGCCATGCCCGGCACCTGAACTGCTT |
| | | | GGTGGTCCCAGCGTGTTCCTGTTCCCGCCGAAGCCTAAAGATACTC |
| | | | TAATGATCAGCCGTACGCCAGAGGTGACATGTGTCGTGGTTGACG |
| | | | TGTCCCACGAAGATCCCGAAGTTAAGTTCAATTGGTATGTTGATGG |
| | | | TGTAGAGGTACACAATGCTAAGACTAAACCTCGCGAGGAGCAGTA |
| | | | CGCCTCGACCTATCGTGTCGTGAGCGTTCTGACCGTCCTTCACCAA |
| | | | GATTGGCTTAACGGCAAAGAATATAAGTGCAAGGTAAGCAATAAA |
| | | | GCACTTCCGGCCCCAATCGAGAAAACCATTTCCAAGGCCAAAGGT |
| | | | CAACCAAGAGAACCCCAGGTGTATACTCTTCCGCCTTCTCGTGAGG |
| | | | AAATGACTAAAAATCAAGTATCCCTTACGTGTCTGGTTAAAGGTTT |
| | | | TTATCCTAGCGATATTGCTGTTGAATGGGAATCGAACGGTCAGCCG |
| | | | GAGAATAATTATAAAACAACGCCACCCGTCCTGGATAGCGACGGC |
| | | | TCATTTTTTCTGTATAGCAAACTGACTGTAGATAAATCACGGTGGC |
| | | | AGCAGGGCAATGTATTCAGTTGCTCCGTTATGCATGAAGCGTTACA |
| | | | TAATCACTACACGCAGAAATCTCTTAGTCTTTCACCCGGT |
| 550 | ATX-P-594 | HC IgG1 Fc- | CAGGTACAGCTGCAGCAGTCAGGTCCAGGACTGGTGAAGCCCTCG |
| | | nucleotide | CAGACCCTCTCACTCACCTGTGCCATCTCCGGGGACAGTGTCTCTA |
| | | | GTAACAGTGCTTCTTGGAACTGGATCAGGCAGTCCCCTTCGAGAG |
| | | | GCCTTGAGTGGCTGGGAACGACATACTACAGGTCCAAGTGGTTTA |
| | | | ATGTTTATGCAGTCTCTGTGAAAAGTCGAATAACCATCAACCCAG |
| | | | ACACATCCAAGAACCAATTCTCCCTGCACCTGAACTCTGTGACTCC |
| | | | CGAGGACACGGCTATATATTTCTGTACAAGAGATCGGGGGGACCT |
| | | | ACTCCACTGGTACTTCGATCTCTGGGGCCGTGGCACCCTGGTCACT |
| | | | GTCTCCTCAGCTAGCACTAAAGGGCCTTCTGTATTTCCCTTGGCCC |
| | | | CGTCCAGCAAATCGACCTCGGGAGGGACAGCCGCCCTGGGTTGCC |
| | | | TTGTGAAAGATTATTTCCCTGAGCCAGTTACCGTAAGTTGGAACAG |
| | | | TGGGGCGCTGACAAGTGGTGTGCACACGTTTCCTGCCGTCCTGCAA |
| | | | TCATCGGGCTTGTATAGCCTCAGCTCTGTGGTCACTGTCCCAAGTT |
| | | | CATCGCTGGGCACTCAGACGTATATTTGCAATGTGAACCACAAAC |
| | | | CTTCAAATACAAAAGTGGATAAACGCGTAGAACCGAAATCGTGTG |
| | | | ATAAAACTCACACATGCCCGCCATGCCCGGCACCTGAACTGCTTG |
| | | | GTGGTCCCAGCGTGTTCCTGTTCCCGCCGAAGCCTAAAGATACTCT |
| | | | AATGATCAGCCGTACGCCAGAGGTGACATGTGTCGTGGTTGACGT |
| | | | GTCCCACGAAGATCCCGAAGTTAAGTTCAATTGGTATGTTGATGGT |
| | | | GTAGAGGTACACAATGCTAAGACTAAACCTCGCGAGGAGCAGTAC |
| | | | GCCTCGACCTATCGTGTCGTGAGCGTTCTGACCGTCCTTCACCAAG |
| | | | ATTGGCTTAACGGCAAAGAATATAAGTGCAAGGTAAGCAATAAAG |
| | | | CACTTCCGGCCCCAATCGAGAAAACCATTTCCAAGGCCAAAGGTC |
| | | | AACCAAGAGAACCCCAGGTGTATACTCTTCCGCCTTCTCGTGAGG |
| | | | AAATGACTAAAAATCAAGTATCCCTTACGTGTCTGGTTAAAGGTTT |
| | | | TTATCCTAGCGATATTGCTGTTGAATGGGAATCGAACGGTCAGCCG |
| | | | GAGAATAATTATAAAACAACGCCACCCGTCCTGGATAGCGACGGC |
| | | | TCATTTTTTCTGTATAGCAAACTGACTGTAGATAAATCACGGTGGC |
| | | | AGCAGGGCAATGTATTCAGTTGCTCCGTTATGCATGAAGCGTTACA |
| | | | TAATCACTACACGCAGAAATCTCTTAGTCTTTCACCCGGT |
| 551 | ATX-P-595 | HC IgG1 Fc- | CAGGTACAGCTGCAGCAGTCGGGTCCAGGACTGGTGAAGCCCTCG |
| | | nucleotide | CAGACCCTCTCACTCACCTGTGCCATCTCCGGGGACAGTGTCTCTA |
| | | | GCAACAGTGCTGCTTGGAACTGGATCAGGCAGTCCCCATCGAGAG |
| | | | GCCTTGAGTGGCTGGGAAGGACATACTACAGGTCCAAGTGGTTTA |
| | | | ATGATTATGCACTATCTGTGAAAAGTCGAATAACCATCAACCCAG |
| | | | ACACATCCAAGAACCAGTTCTCCCTGCACCTGAACTCTGTGACTCC |
| | | | CGAGGACACGGCTGTGTATTACTGTGCAAGAGAACCTGGTATAGT |
| | | | AGCACCTGGCCCTTTTGACTACTGGGGCCAGGGAACCCTGGTCAC |
| | | | CGTCTCCTCAGCTAGCACTAAAGGGCCTTCTGTATTTCCCTTGGCC |
| | | | CCGTCCAGCAAATCGACCTCGGGAGGGACAGCCGCCCTGGGTTGC |
| | | | CTTGTGAAAGATTATTTCCCTGAGCCAGTTACCGTAAGTTGGAACA |
| | | | GTGGGGCGCTGACAAGTGGTGTGCACACGTTTCCTGCCGTCCTGCA |
| | | | ATCATCGGGCTTGTATAGCCTCAGCTCTGTGGTCACTGTCCCAAGT |
| | | | TCATCGCTGGGCACTCAGACGTATATTTGCAATGTGAACCACAAA |
| | | | CCTTCAAATACAAAAGTGGATAAACGCGTAGAACCGAAATCGTGT |
| | | | GATAAAACTCACACATGCCCGCCATGCCCGGCACCTGAACTGCTT |
| | | | GGTGGTCCCAGCGTGTTCCTGTTCCCGCCGAAGCCTAAAGATACTC |
| | | | TAATGATCAGCCGTACGCCAGAGGTGACATGTGTCGTGGTTGACG |
| | | | TGTCCCACGAAGATCCCGAAGTTAAGTTCAATTGGTATGTTGATGG |
| | | | TGTAGAGGTACACAATGCTAAGACTAAACCTCGCGAGGAGCAGTA |
| | | | CGCCTCGACCTATCGTGTCGTGAGCGTTCTGACCGTCCTTCACCAA |
| | | | GATTGGCTTAACGGCAAAGAATATAAGTGCAAGGTAAGCAATAAA |
| | | | GCACTTCCGGCCCCAATCGAGAAAACCATTTCCAAGGCCAAAGGT |
| | | | CAACCAAGAGAACCCCAGGTGTATACTCTTCCGCCTTCTCGTGAGG |
| | | | AAATGACTAAAAATCAAGTATCCCTTACGTGTCTGGTTAAAGGTTT |
| | | | TTATCCTAGCGATATTGCTGTTGAATGGGAATCGAACGGTCAGCCG |
| | | | GAGAATAATTATAAAACAACGCCACCCGTCCTGGATAGCGACGGC |
| | | | TCATTTTTTCTGTATAGCAAACTGACTGTAGATAAATCACGGTGGC |
| | | | AGCAGGGCAATGTATTCAGTTGCTCCGTTATGCATGAAGCGTTACA |
| | | | TAATCACTACACGCAGAAATCTCTTAGTCTTTCACCCGGT |
| 552 | ATX-P-604 | HC IgG1 Fc- | CAGGTACAGCTGCAGCAGTCAGGTCCAGGACTGGTGAAGCCCACG |
| | | nucleotide | CAGACCCTCTCACTCACCTGTGCCATCTCCGGGGACAGTGTCTCTA |
| | | | GCAACAGTGCTGCATGGAACTGGATCAGGCAGTCCCCATCGAGAG |
| | | | GCCTTGAGTGGCTGGGGAGGACATACTACAGGTCCAAGTGGTTTA |
| | | | ATGGTTATGCAGTATCTGTGAAAAGTCGAATAACCATCAACCCCG |
| | | | ACGCATCCAAGAACCAGTTCTCCCTGCACTTGAAGTCTGTGACTGA |
| | | | CGAGGACACGGCTGTATATTACTGTGCAACAGATCGGGGGGAACT |
| | | | CCTCCACTGGTACTTCGATCTCTGGGGCCGTGGCACCCTGGTCACC |
| | | | GTCTCTTCAGCTAGCACTAAAGGGCCTTCTGTATTTCCCTTGGCCC |
| | | | CGTCCAGCAAATCGACCTCGGGAGGGACAGCCGCCCTGGGTTGCC |
| | | | TTGTGAAAGATTATTTCCCTGAGCCAGTTACCGTAAGTTGGAACAG |
| | | | TGGGGCGCTGACAAGTGGTGTGCACACGTTTCCTGCCGTCCTGCAA |
| | | | TCATCGGGCTTGTATAGCCTCAGCTCTGTGGTCACTGTCCCAAGTT |
| | | | CATCGCTGGGCACTCAGACGTATATTTGCAATGTGAACCACAAAC |
| | | | CTTCAAATACAAAAGTGGATAAACGCGTAGAACCGAAATCGTGTG |
| | | | ATAAAACTCACACATGCCCGCCATGCCCGGCACCTGAACTGCTTG |
| | | | GTGGTCCCAGCGTGTTCCTGTTCCCGCCGAAGCCTAAAGATACTCT |
| | | | AATGATCAGCCGTACGCCAGAGGTGACATGTGTCGTGGTTGACGT |
| | | | GTCCCACGAAGATCCCGAAGTTAAGTTCAATTGGTATGTTGATGGT |
| | | | GTAGAGGTACACAATGCTAAGACTAAACCTCGCGAGGAGCAGTAC |
| | | | GCCTCGACCTATCGTGTCGTGAGCGTTCTGACCGTCCTTCACCAAG |
| | | | ATTGGCTTAACGGCAAAGAATATAAGTGCAAGGTAAGCAATAAAG |
| | | | CACTTCCGGCCCCAATCGAGAAAACCATTTCCAAGGCCAAAGGTC |
| | | | AACCAAGAGAACCCCAGGTGTATACTCTTCCGCCTTCTCGTGAGG |
| | | | AAATGACTAAAAATCAAGTATCCCTTACGTGTCTGGTTAAAGGTTT |
| | | | TTATCCTAGCGATATTGCTGTTGAATGGGAATCGAACGGTCAGCCG |
| | | | GAGAATAATTATAAAACAACGCCACCCGTCCTGGATAGCGACGGC |
| | | | TCATTTTTTCTGTATAGCAAACTGACTGTAGATAAATCACGGTGGC |
| | | | AGCAGGGCAATGTATTCAGTTGCTCCGTTATGCATGAAGCGTTACA |
| | | | TAATCACTACACGCAGAAATCTCTTAGTCTTTCACCCGGT |
| 553 | ATX-P-610 | HC IgG1 Fc- | CAGGTACAGCTGCAGCAGTCAGGTCCAGGACTGGTGAAGTCCTCG |
| | | nucleotide | CAGACCCTCTCAATCACCTGTGCCATCTCCGGGGACAGTGTCTCTA |
| | | | GCAACAGTGCTGCTTGGAACTGGATCAGGCAGTCCCCATCGAGAG |
| | | | GCCTTGAGTGGCTGGGAAGGACATACTACAGGTCCAAGTGGTATA |
| | | | ATGATTATGCACTATCTGTGAAAAGTCGAATAATCATCAAGTCAG |
| | | | ACACATCCAAGAACCAGTTCTCCCTGCAGTTGAACTCTGTGACTCC |
| | | | CGAGGACACGGCTGTGTACTACTGTGCAAGAGAACCGGGTATAGC |
| | | | AGCACCTGGTCCATTTGACTACTGGGGCCAGGGATCCCTGGTCACC |
| | | | GTCTCCTCAGCTAGCACTAAAGGGCCTTCTGTATTTCCCTTGGCCC |
| | | | CGTCCAGCAAATCGACCTCGGGAGGGACAGCCGCCCTGGGTTGCC |
| | | | TTGTGAAAGATTATTTCCCTGAGCCAGTTACCGTAAGTTGGAACAG |
| | | | TGGGGCGCTGACAAGTGGTGTGCACACGTTTCCTGCCGTCCTGCAA |
| | | | TCATCGGGCTTGTATAGCCTCAGCTCTGTGGTCACTGTCCCAAGTT |
| | | | CATCGCTGGGCACTCAGACGTATATTTGCAATGTGAACCACAAAC |
| | | | CTTCAAATACAAAAGTGGATAAACGCGTAGAACCGAAATCGTGTG |
| | | | ATAAAACTCACACATGCCCGCCATGCCCGGCACCTGAACTGCTTG |
| | | | GTGGTCCCAGCGTGTTCCTGTTCCCGCCGAAGCCTAAAGATACTCT |
| | | | AATGATCAGCCGTACGCCAGAGGTGACATGTGTCGTGGTTGACGT |
| | | | GTCCCACGAAGATCCCGAAGTTAAGTTCAATTGGTATGTTGATGGT |
| | | | GTAGAGGTACACAATGCTAAGACTAAACCTCGCGAGGAGCAGTAC |
| | | | GCCTCGACCTATCGTGTCGTGAGCGTTCTGACCGTCCTTCACCAAG |
| | | | ATTGGCTTAACGGCAAAGAATATAAGTGCAAGGTAAGCAATAAAG |
| | | | CACTTCCGGCCCCAATCGAGAAAACCATTTCCAAGGCCAAAGGTC |
| | | | AACCAAGAGAACCCCAGGTGTATACTCTTCCGCCTTCTCGTGAGG |
| | | | AAATGACTAAAAATCAAGTATCCCTTACGTGTCTGGTTAAAGGTTT |
| | | | TTATCCTAGCGATATTGCTGTTGAATGGGAATCGAACGGTCAGCCG |
| | | | GAGAATAATTATAAAACAACGCCACCCGTCCTGGATAGCGACGGC |
| | | | TCATTTTTTCTGTATAGCAAACTGACTGTAGATAAATCACGGTGGC |
| | | | AGCAGGGCAATGTATTCAGTTGCTCCGTTATGCATGAAGCGTTACA |
| | | | TAATCACTACACGCAGAAATCTCTTAGTCTTTCACCCGGT |
| 554 | ATX-P-560 | LC Kappa | DIQMTQSPSSVSTSVGDRVTITCRASQGISSWLAWYQQKPGKAPKLLI |
| | | | SAASSLQSGVPSRFSGSGSGTDFTLTISTLQPEDFASYYCQQANSFPPTF |
| | | | GQGTRVEIKRTVAAPSVFIFPPSDEQLKSGTASVVCLLNNFYPREAKV |
| | | | QWKVDNALQSGNSQESVTEQDSKDSTYSLSSTLTLSKADYEKHKVY |
| | | | ACEVTHQGLSSPVTKSFNRGEC |
| 555 | ATX-P-561 | LC Kappa | DIQMTQSPSSLSASVGDRVTITCRASQGISSWLTWYQQKPGKAPKLLI |
| | | | YAASSLQGGVPSRFSGSGSGTDFTLTISSLQPEDFATYYCQQANSFPPT |
| | | | FGQGTKVEIKRTVAAPSVFIFPPSDEQLKSGTASVVCLLNNFYPREAK |
| | | | VQWKVDNALQSGNSQESVTEQDSKDSTYSLSSTLTLSKADYEKHKV |
| | | | YACEVTHQGLSSPVTKSFNRGEC |
| 556 | ATX-P-562 | LC Kappa | DIQMTQSPSSVSASIGDRVTITCRASKGISIWLAWYQQKPGKAPKVLIS |
| | | | AASSLQSGAPSRFSGSGSGTDFTLTISSLQPEDFATYYCQQANSFPPTF |
| | | | GQGTKVEIKRTVAAPSVFIFPPSDEQLKSGTASVVCLLNNFYPREAKV |
| | | | QWKVDNALQSGNSQESVTEQDSKDSTYSLSSTLTLSKADYEKHKVY |
| | | | ACEVTHQGLSSPVTKSFNRGEC |
| 557 | ATX-P-566 | LC Kappa | EIVLTQSPGTLSLSPGDRATLSCRASQSVRNSYVAWYQQKPGQAPRLL |
| | | | IYGASSRATGIPDRFSGSGSGTDFSLTIGRLEPEDFVVYYCQQYGNSPL |
| | | | TFGGGTKVEIKRTVAAPSVFIFPPSDEQLKSGTASVVCLLNNFYPREA |
| | | | KVQWKVDNALQSGNSQESVTEQDSKDSTYSLSSTLTLSKADYEKHK |
| | | | VYACEVTHQGLSSPVTKSFNRGEC |
| 558 | ATX-P-572 | LC Kappa | DIVMTQSPDSLAVSLGERATINCKSSQSVLYSSNNKNYLAWYQQKPG |
| | | | QPPKLLIYWASPRESGVPDRFSGSGSGTDFTLTISSLQAEDVAVYYCQ |
| | | | QYYSIPRTFGQGTKVEIKRTVAAPSVFIFPPSDEQLKSGTASVVCLLNN |
| | | | FYPREAKVQWKVDNALQSGNSQESVTEQDSKDSTYSLSSTLTLSKAD |
| | | | YEKHKVYACEVTHQGLSSPVTKSFNRGEC |
| 559 | ATX-P-573 | LC Kappa | DIQMTQSPSSVSASVGDRVTITCRASQGISSWLAWYQQKPGKAPKVLI |
| | | | YAASSLQSGVPSRFSGSGSGTDFTLTISSLQPEDFATYYCQQANSFPPT |
| | | | FGPGTKVDIKRTVAAPSVFIFPPSDEQLKSGTASVVCLLNNFYPREAK |
| | | | VQWKVDNALQSGNSQESVTEQDSKDSTYSLSSTLTLSKADYEKHKV |
| | | | YACEVTHQGLSSPVTKSFNRGEC |
| 560 | ATX-P-581 | LC Kappa | DIQMTQSPSSVSASVGDRVTITCRASQGVNNWLAWYQQKPGKAPKL |
| | | | LIHAASSLLSGVPSRFSGSGSGTDFTLTITSLQPEDFATYYCQQANSFPI |
| | | | TFGQGTRLEIKRTVAAPSVFIFPPSDEQLKSGTASVVCLLNNFYPREAK |
| | | | VQWKVDNALQSGNSQESVTEQDSKDSTYSLSSTLTLSKADYEKHKV |
| | | | YACEVTHQGLSSPVTKSFNRGEC |
| 561 | ATX-P-587 | LC Kappa | DIVMTQSPDSLAVSLGERATINCKSSQSVLYSSNNKNYLAWYQQKPG |
| | | | QPPKLLIYWASTRESGVPDRFSGSGSGTDFTLTISSLQAEDVAVYYCQ |
| | | | QYYSTPYTFGQGTKLEIKRTVAAPSVFIFPPSDEQLKSGTASVVCLLNN |
| | | | FYPREAKVQWKVDNALQSGNSQESVTEQDSKDSTYSLSSTLTLSKAD |
| | | | YEKHKVYACEVTHQGLSSPVTKSFNRGEC |
| 562 | ATX-P-592 | LC Kappa | DIVMTQSPDSLAVSLGERATINCKSSQSVLYSSNNKNYLAWYQQKPG |
| | | | QPPKLLIYWASNRESGVPDRFSGSGSGTDFTLTISSLQAEDVAVYYCQ |
| | | | QYYSTPYTFGQGTKLEIKRTVAAPSVFIFPPSDEQLKSGTASVVCLLNN |
| | | | FYPREAKVQWKVDNALQSGNSQESVTEQDSKDSTYSLSSTLTLSKAD |
| | | | YEKHKVYACEVTHQGLSSPVTKSFNRGEC |
| 563 | ATX-P-594 | LC Kappa | EIVLTQSPGTLSLSPGERATLSCRASQSVSSSYLAWYQQKPGQSPRLLI |
| | | | YGASSRATGIPDRFSGSGSGTDFTLTISRLEPEDFAVYYCQQYGRSVTF |
| | | | GGGTKLEIKRTVAAPSVFIFPPSDEQLKSGTASVVCLLNNFYPREAKV |
| | | | QWKVDNALQSGNSQESVTEQDSKDSTYSLSSTLTLSKADYEKHKVY |
| | | | ACEVTHQGLSSPVTKSFNRGEC |
| 564 | ATX-P-595 | LC Kappa | DIVMTQSPDSLAVSLGERATINCKSSQSVLYSSNNKNYLAWYQQKPG |
| | | | QPPKLLIYWASTRESGVPDRFSGSGSGTDFTLTISSLQAEDVAVYYCQ |
| | | | QYYSTPYTFGQGTKLEIKRTVAAPSVFIFPPSDEQLKSGTASVVCLLNN |
| | | | FYPREAKVQWKVDNALQSGNSQESVTEQDSKDSTYSLSSTLILSKAD |
| | | | YEKHKVYACEVTHQGLSSPVTKSFNRGEC |
| 565 | ATX-P-604 | LC Kappa | EIVLTQSPDTLSLSPGERATLSCRASQTIRSSYLAWYQLKPGQAPRLLI |
| | | | YGASSRATGIPDRFSGSGSGTDFTLSISRLEPEDFAVYYCQQYGRSIIFG |
| | | | GGTKVEIKRTVAAPSVFIFPPSDEQLKSGTASVVCLLNNFYPREAKVQ |
| | | | WKVDNALQSGNSQESVTEQDSKDSTYSLSSTLTLSKADYEKHKVYA |
| | | | CEVTHQGLSSPVIKSFNRGEC |
| 566 | ATX-P-610 | LC Kappa | DIQMTQSPSSVSASVGDRVTITCRASQGISSWLAWYQQKPGKAPKLLI |
| | | | YAASSLQSGVPSRFSGSGSGTDFTLTISSLQPEDFATYYCQQNYRTPIT |
| | | | FGQGTRLEIKRTVAAPSVFIFPPSDEQLKSGTASVVCLLNNFYPREAKV |
| | | | QWKVDNALQSGNSQESVTEQDSKDSTYSLSSTLTLSKADYEKHKVY |
| | | | ACEVTHQGLSSPVTKSFNRGEC |
| 567 | ATX-P-560 | LC Kappa- | GACATCCAGATGACCCAGTCTCCATCTTCCGTGTCTACATCTGTTG |
| | | nucleotide | GAGACAGAGTCACCATCACTTGTCGGGCGAGTCAGGGTATTAGCA |
| | | | GCTGGTTAGCCTGGTATCAGCAGAAACCAGGGAAAGCCCCTAAGC |
| | | | TCCTGATCTCTGCTGCATCCAGTTTGCAAAGTGGGGTCCCATCAAG |
| | | | GTTCAGCGGCAGTGGATCTGGGACAGATTTCACTCTCACCATCAGC |
| | | | ACCCTGCAGCCTGAAGACTTTGCATCTTACTATTGTCAACAGGCTA |
| | | | ACAGTTTCCCTCCGACGTTCGGCCAAGGGACCAGGGTGGAAATCA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 568 | ATX-P-561 | LC Kappa- | GACATCCAGATGACCCAGTCTCCATCTTCCCTGTCTGCATCTGTCG |
| | | nucleotide | GAGACAGAGTCACCATCACTTGTCGGGCGAGTCAGGGTATTAGCA |
| | | | GCTGGTTAACCTGGTATCAGCAGAAACCAGGGAAAGCCCCTAAGC |
| | | | TCCTGATCTATGCTGCATCCAGTTTGCAAGGTGGGGTCCCATCAAG |
| | | | ATTCAGCGGCAGTGGATCTGGGACAGATTTCACTCTCACCATCAGC |
| | | | AGCCTGCAGCCTGAAGATTTTGCAACTTACTATTGTCAACAGGCTA |
| | | | ACAGTTTCCCTCCGACGTTCGGCCAAGGGACCAAGGTGGAAATCA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 569 | ATX-P-562 | LC Kappa- | GACATCCAGATGACCCAGTCTCCATCTTCCGTGTCTGCATCTATAG |
| | | nucleotide | GAGACAGAGTCACCATCACTTGTCGGGCGAGTAAGGGAATTAGCA |
| | | | TCTGGTTAGCCTGGTATCAGCAGAAACCAGGGAAAGCCCCTAAGG |
| | | | TCCTGATCTCTGCTGCATCCAGTTTGCAAAGTGGGGCCCCATCAAG |
| | | | GTTCAGCGGCAGTGGATCTGGGACAGATTTCACTCTCACCATCAGC |
| | | | AGCCTGCAGCCTGAAGATTTTGCAACTTACTATTGTCAACAGGCTA |
| | | | ACAGTTTCCCTCCGACGTTCGGCCAAGGGACCAAGGTGGAGATCA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |
| 570 | ATX-P-566 | LC Kappa- | GAAATTGTGTTGACGCAGTCTCCAGGCACCCTGTCTTTGTCTCCAG |
| | | nucleotide | GGGACAGAGCCACCCTCTCATGCAGGGCCAGTCAGAGTGTTAGGA |
| | | | ACAGTTACGTAGCCTGGTACCAGCAGAAACCTGGCCAGGCTCCCA |
| | | | GGCTCCTCATCTATGGTGCATCCAGCAGGGCCACTGGCATCCCAG |
| | | | ACAGGTTCAGTGGCAGTGGGTCTGGGACAGACTTCAGTCTCACCA |
| | | | TCGGCAGACTGGAGCCTGAGGATTTTGTAGTGTATTATTGTCAGCA |
| | | | ATACGGTAACTCACCTCTCACTTTCGGCGGAGGGACCAAGGTGGA |
| | | | GATCAAACGTACGGTAGCTGCCCCTTCAGTTTTTATCTTTCCGCCG |
| | | | TCTGACGAGCAGTTAAAATCCGGGACCGCTTCTGTAGTTTGCCTGC |
| | | | TGAATAATTTTTATCCGCGTGAGGCTAAAGTACAATGGAAAGTCG |
| | | | ACAATGCTTTGCAGTCGGGAAATTCACAGGAAAGTGTTACGGAGC |
| | | | AGGATTCTAAAGATTCCACATATTCACTCAGCTCCACCCTTACACT |
| | | | GAGCAAAGCCGACTATGAAAAACATAAAGTTTACGCATGTGAGGT |
| | | | GACGCACCAAGGATTATCCAGTCCGGTCACAAAATCGTTTAACCG |
| | | | CGGTGAGTGT |
| 571 | ATX-P-572 | LC Kappa- | GACATCGTGATGACCCAGTCTCCAGACTCCCTGGCTGTGTCTCTGG |
| | | nucleotide | GCGAGAGGGCCACCATCAACTGCAAGTCCAGCCAGAGTGTTTTAT |
| | | | ACAGCTCCAACAATAAGAACTACTTAGCTTGGTACCAGCAGAAAC |
| | | | CTGGACAGCCTCCTAAGCTGCTCATTTACTGGGCATCTCCCCGGGA |
| | | | ATCCGGGGTCCCTGACCGGTTCAGTGGCAGCGGGTCTGGGACAGA |
| | | | TTTCACTCTCACCATCAGCAGCCTGCAGGCTGAAGATGTGGCAGTT |
| | | | TATTATTGTCAGCAATATTATAGTATTCCTCGGACGTTCGGCCAAG |
| | | | GGACCAAGGTGGAAATCAAACGTACGGTAGCTGCCCCTTCAGTTT |
| | | | TTATCTTTCCGCCGTCTGACGAGCAGTTAAAATCCGGGACCGCTTC |
| | | | TGTAGTTTGCCTGCTGAATAATTTTTATCCGCGTGAGGCTAAAGTA |
| | | | CAATGGAAAGTCGACAATGCTTTGCAGTCGGGAAATTCACAGGAA |
| | | | AGTGTTACGGAGCAGGATTCTAAAGATTCCACATATTCACTCAGCT |
| | | | CCACCCTTACACTGAGCAAAGCCGACTATGAAAAACATAAAGTTT |
| | | | ACGCATGTGAGGTGACGCACCAAGGATTATCCAGTCCGGTCACAA |
| | | | AATCGTTTAACCGCGGTGAGTGT |
| 572 | ATX-P-573 | LC Kappa- | GACATCCAGATGACCCAGTCTCCATCTTCCGTGTCTGCATCTGTAG |
| | | nucleotide | GAGACAGAGTCACCATCACTTGTCGGGCGAGTCAGGGTATTAGCA |
| | | | GCTGGTTAGCCTGGTATCAGCAGAAACCAGGGAAAGCCCCTAAGG |
| | | | TCCTGATCTATGCTGCATCCAGTTTGCAAAGTGGGGTCCCATCAAG |
| | | | GTTCAGCGGCAGTGGATCTGGGACAGATTTCACTCTCACCATCAGC |
| | | | AGCCTGCAGCCTGAAGATTTTGCAACTTACTATTGTCAACAGGCTA |
| | | | ACAGTTTCCCTCCCACTTTCGGCCCTGGGACCAAAGTGGATATCAA |
| | | | ACGTACGGTAGCTGCCCCTTCAGTTTTTATCTTTCCGCCGTCTGAC |
| | | | GAGCAGTTAAAATCCGGGACCGCTTCTGTAGTTTGCCTGCTGAATA |
| | | | ATTTTTATCCGCGTGAGGCTAAAGTACAATGGAAAGTCGACAATG |
| | | | CTTTGCAGTCGGGAAATTCACAGGAAAGTGTTACGGAGCAGGATT |
| | | | CTAAAGATTCCACATATTCACTCAGCTCCACCCTTACACTGAGCAA |
| | | | AGCCGACTATGAAAAACATAAAGTTTACGCATGTGAGGTGACGCA |
| | | | CCAAGGATTATCCAGTCCGGTCACAAAATCGTTTAACCGCGGTGA |
| | | | GTGT |
| 573 | ATX-P-581 | LC Kappa- | GACATCCAGATGACTCAGTCTCCATCTTCCGTGTCTGCATCTGTAG |
| | | nucleotide | GAGACAGAGTCACCATCACTTGTCGGGCGAGTCAGGGTGTTAACA |
| | | | ACTGGTTAGCCTGGTATCAGCAGAAACCAGGGAAAGCCCCTAAGC |
| | | | TCCTGATCCATGCTGCATCCAGTTTGCTAAGTGGGGTCCCATCAAG |
| | | | GTTCAGCGGCAGTGGATCTGGGACAGATTTCACGCTCACCATCAC |
| | | | CAGCCTGCAGCCTGAAGATTTTGCAACTTACTATTGTCAACAGGCT |
| | | | AACAGTTTCCCCATCACCTTCGGCCAAGGGACACGACTGGAAATT |
| | | | AAACGTACGGTAGCTGCCCCTTCAGTTTTTATCTTTCCGCCGTCTG |
| | | | ACGAGCAGTTAAAATCCGGGACCGCTTCTGTAGTTTGCCTGCTGAA |
| | | | TAATTTTTATCCGCGTGAGGCTAAAGTACAATGGAAAGTCGACAA |
| | | | TGCTTTGCAGTCGGGAAATTCACAGGAAAGTGTTACGGAGCAGGA |
| | | | TTCTAAAGATTCCACATATTCACTCAGCTCCACCCTTACACTGAGC |
| | | | AAAGCCGACTATGAAAAACATAAAGTTTACGCATGTGAGGTGACG |
| | | | CACCAAGGATTATCCAGTCCGGTCACAAAATCGTTTAACCGCGGT |
| | | | GAGTGT |
| 574 | ATX-P-587 | LC Kappa- | GACATCGTGATGACCCAGTCTCCAGACTCCCTGGCTGTGTCTCTGG |
| | | nucleotide | GCGAGAGGGCCACCATCAACTGCAAGTCCAGCCAGAGTGTTTTAT |
| | | | ACAGCTCCAACAATAAGAACTACTTAGCTTGGTACCAGCAGAAAC |
| | | | CAGGACAGCCTCCTAAGCTGCTCATTTACTGGGCATCTACCCGGGA |
| | | | ATCCGGGGTCCCTGACCGATTCAGTGGCAGCGGGTCTGGGACAGA |
| | | | TTTCACTCTCACCATCAGCAGCCTGCAGGCTGAAGATGTGGCAGTT |
| | | | TATTACTGTCAGCAATATTATAGTACTCCGTACACTTTTGGCCAGG |
| | | | GGACCAAGCTGGAGATCAAACGTACGGTAGCTGCCCCTTCAGTTT |
| | | | TTATCTTTCCGCCGTCTGACGAGCAGTTAAAATCCGGGACCGCTTC |
| | | | TGTAGTTTGCCTGCTGAATAATTTTTATCCGCGTGAGGCTAAAGTA |
| | | | CAATGGAAAGTCGACAATGCTTTGCAGTCGGGAAATTCACAGGAA |
| | | | AGTGTTACGGAGCAGGATTCTAAAGATTCCACATATTCACTCAGCT |
| | | | CCACCCTTACACTGAGCAAAGCCGACTATGAAAAACATAAAGTTT |
| | | | ACGCATGTGAGGTGACGCACCAAGGATTATCCAGTCCGGTCACAA |
| | | | AATCGTTTAACCGCGGTGAGTGT |
| 575 | ATX-P-592 | LC Kappa- | GACATCGTGATGACCCAGTCTCCAGACTCCCTGGCTGTGTCTCTGG |
| | | nucleotide | GCGAGAGGGCCACCATCAACTGCAAGTCCAGCCAGAGTGTTTTAT |
| | | | ACAGCTCCAACAATAAGAACTACTTAGCTTGGTACCAGCAGAAAC |
| | | | CAGGACAGCCTCCTAAGCTGCTCATTTACTGGGCATCTAACCGGG |
| | | | AATCCGGGGTCCCTGACCGATTCAGTGGCTCCGGGTCTGGGACAG |
| | | | ATTTCACTCTCACCATCAGCAGCCTGCAGGCTGAAGATGTGGCAGT |
| | | | TTATTACTGTCAGCAATATTATAGTACTCCGTACACTTTTGGCCAG |
| | | | GGGACCAAGCTGGAGATCAAACGTACGGTAGCTGCCCCTTCAGTT |
| | | | TTTATCTTTCCGCCGTCTGACGAGCAGTTAAAATCCGGGACCGCTT |
| | | | CTGTAGTTTGCCTGCTGAATAATTTTTATCCGCGTGAGGCTAAAGT |
| | | | ACAATGGAAAGTCGACAATGCTTTGCAGTCGGGAAATTCACAGGA |
| | | | AAGTGTTACGGAGCAGGATTCTAAAGATTCCACATATTCACTCAG |
| | | | CTCCACCCTTACACTGAGCAAAGCCGACTATGAAAAACATAAAGT |
| | | | TTACGCATGTGAGGTGACGCACCAAGGATTATCCAGTCCGGTCAC |
| | | | AAAATCGTTTAACCGCGGTGAGTGT |
| 576 | ATX-P-594 | LC Kappa- | GAAATTGTGTTGACGCAGTCTCCAGGCACCCTGTCTTTGTCTCCAG |
| | | nucleotide | GGGAGAGAGCCACCCTCTCCTGCAGGGCCAGTCAGAGTGTTAGCA |
| | | | GCAGCTACTTAGCCTGGTACCAGCAGAAACCTGGCCAGTCTCCCA |
| | | | GGCTCCTCATCTATGGTGCATCCAGCAGGGCCACTGGCATCCCAG |
| | | | ACAGGTTCAGTGGCAGTGGGTCTGGGACAGACTTCACTCTCACCA |
| | | | TCAGCAGACTGGAGCCTGAGGATTTTGCAGTATATTACTGTCAGCA |
| | | | GTATGGTAGGTCAGTCACTTTCGGCGGAGGGACCAAGCTGGAAAT |
| | | | CAAACGTACGGTAGCTGCCCCTTCAGTTTTTATCTTTCCGCCGTCT |
| | | | GACGAGCAGTTAAAATCCGGGACCGCTTCTGTAGTTTGCCTGCTGA |
| | | | ATAATTTTTATCCGCGTGAGGCTAAAGTACAATGGAAAGTCGACA |
| | | | ATGCTTTGCAGTCGGGAAATTCACAGGAAAGTGTTACGGAGCAGG |
| | | | ATTCTAAAGATTCCACATATTCACTCAGCTCCACCCTTACACTGAG |
| | | | CAAAGCCGACTATGAAAAACATAAAGTTTACGCATGTGAGGTGAC |
| | | | GCACCAAGGATTATCCAGTCCGGTCACAAAATCGTTTAACCGCGG |
| | | | TGAGTGT |
| 577 | ATX-P-595 | LC Kappa- | GACATCGTGATGACCCAGTCTCCAGACTCCCTGGCTGTGTCTCTGG |
| | | nucleotide | GCGAGAGGGCCACCATCAACTGCAAGTCCAGCCAGAGTGTTTTAT |
| | | | ACAGCTCCAACAATAAGAACTACTTAGCTTGGTACCAGCAGAAAC |
| | | | CAGGACAGCCTCCTAAGCTGCTCATTTACTGGGCATCTACCCGGGA |
| | | | ATCCGGGGTCCCTGACCGATTCAGTGGCAGCGGGTCTGGGACAGA |
| | | | TTTCACTCTCACCATCAGCAGCCTGCAGGCTGAAGATGTGGCAGTT |
| | | | TATTACTGTCAGCAATATTATAGTACTCCGTACACTTTTGGCCAGG |
| | | | GGACCAAGCTGGAAATCAAACGTACGGTAGCTGCCCCTTCAGTTT |
| | | | TTATCTTTCCGCCGTCTGACGAGCAGTTAAAATCCGGGACCGCTTC |
| | | | TGTAGTTTGCCTGCTGAATAATTTTTATCCGCGTGAGGCTAAAGTA |
| | | | CAATGGAAAGTCGACAATGCTTTGCAGTCGGGAAATTCACAGGAA |
| | | | AGTGTTACGGAGCAGGATTCTAAAGATTCCACATATTCACTCAGCT |
| | | | CCACCCTTACACTGAGCAAAGCCGACTATGAAAAACATAAAGTTT |
| | | | ACGCATGTGAGGTGACGCACCAAGGATTATCCAGTCCGGTCACAA |
| | | | AATCGTTTAACCGCGGTGAGTGT |
| 578 | ATX-P-604 | LC Kappa- | GAAATAGTGCTGACTCAGTCTCCAGACACCCTATCTTTGTCTCCAG |
| | | nucleotide | GGGAAAGAGCCACCCTCTCCTGCAGGGCCAGTCAGACTATTAGGA |
| | | | GCAGCTACTTAGCCTGGTACCAGCTGAAACCTGGCCAGGCTCCCA |
| | | | GGCTCCTCATCTATGGTGCATCCAGCAGGGCCACTGGCATCCCAG |
| | | | ACAGGTTCAGTGGCAGTGGGTCTGGGACAGACTTCACTCTCTCCAT |
| | | | CAGCAGACTGGAGCCTGAAGATTTTGCAGTGTATTACTGTCAACA |
| | | | GTATGGCCGCTCAATCATTTTCGGCGGAGGGACCAAAGTGGAAAT |
| | | | CAAACGTACGGTAGCTGCCCCTTCAGTTTTTATCTTTCCGCCGTCT |
| | | | GACGAGCAGTTAAAATCCGGGACCGCTTCTGTAGTTTGCCTGCTGA |
| | | | ATAATTTTTATCCGCGTGAGGCTAAAGTACAATGGAAAGTCGACA |
| | | | ATGCTTTGCAGTCGGGAAATTCACAGGAAAGTGTTACGGAGCAGG |
| | | | ATTCTAAAGATTCCACATATTCACTCAGCTCCACCCTTACACTGAG |
| | | | CAAAGCCGACTATGAAAAACATAAAGTTTACGCATGTGAGGTGAC |
| | | | GCACCAAGGATTATCCAGTCCGGTCACAAAATCGTTTAACCGCGG |
| | | | TGAGTGT |
| 579 | ATX-P-610 | LC Kappa- | GACATCCAGATGACCCAGTCTCCATCTTCCGTGTCTGCATCTGTAG |
| | | nucleotide | GAGACAGAGTCACCATCACTTGTCGGGCGAGTCAGGGTATTAGCA |
| | | | GCTGGTTAGCCTGGTATCAGCAGAAACCAGGGAAAGCCCCTAAGC |
| | | | TCCTGATCTATGCTGCATCCAGTTTGCAAAGTGGGGTCCCATCAAG |
| | | | GTTCAGCGGCAGTGGATCTGGGACAGATTTCACTCTCACCATCAGC |
| | | | AGCCTGCAGCCTGAAGATTTTGCAACTTACTACTGTCAACAGAATT |
| | | | ACAGAACCCCGATCACCTTCGGCCAAGGGACACGACTGGAGATTA |
| | | | AACGTACGGTAGCTGCCCCTTCAGTTTTTATCTTTCCGCCGTCTGA |
| | | | CGAGCAGTTAAAATCCGGGACCGCTTCTGTAGTTTGCCTGCTGAAT |
| | | | AATTTTTATCCGCGTGAGGCTAAAGTACAATGGAAAGTCGACAAT |
| | | | GCTTTGCAGTCGGGAAATTCACAGGAAAGTGTTACGGAGCAGGAT |
| | | | TCTAAAGATTCCACATATTCACTCAGCTCCACCCTTACACTGAGCA |
| | | | AAGCCGACTATGAAAAACATAAAGTTTACGCATGTGAGGTGACGC |
| | | | ACCAAGGATTATCCAGTCCGGTCACAAAATCGTTTAACCGCGGTG |
| | | | AGTGT |

Human complement factor H-related protein 4b (CFHR4b) (see, e.g., UniProt Accession No. Q92496):

(SEQ ID NO: 580)
MLLLINVILTLWVSCANGQEVKPCDFPEIQHGGLYYKSLRRLYFPAAAG
QSYSYYCDQNFVTPSGSYWDYIHCTQDGWSPTVPCLRTCSKSDIEIENG
FISESSSIYILNKEIQYKCKPGYATADGNSSGSITCLQNGWSAQPICIK
FCDMPVFENSRAKSNGMRFKLHDTLDYECYDGYEISYGNTTGSIVCGED
GWSHFPTCYNSSEKCGPPPPISNGDTTSFLLKVYVPQSRVEYQCQSYYE
LQGSNYVTCSNGEWSEPPRCIHPCIITEENMNKNNIQLKGKSDIKYYAK
TGDTIEFMCKLGYNANTSVLSFQAVCREGIVEYPRCE.

Human CFHL1-Y402H-His (ATX-P-421):

(SEQ ID NO: 581)
MYRMQLLSCIALSLALVTNSEDCNELPPRRNTEILTGSWSDQTYPEGTQ
AIYKCRPGYRSLGNVIMVCRKGEWVALNPLRKCQKRPCGHPGDTPFGTF
TLTGGNVFEYGVKAVYTCNEGYQLLGEINYRECDTDGWTNDIPICEVVK
CLPVTAPENGKIVSSAMEPDREYHFGQAVRFVCNSGYKIEGDEEMHCSD
DGFWSKEKPKCVEISCKSPDVINGSPISQKIIYKENERFQYKCNMGYEY
SERGDAVCTESGWRPLPSCEEKSCDNPYIPNGDYSPLRIKHRTGDEITY
QCRNGFYPATRGNTAKCTSTGWIPAPRCTLKPCDYPDIKHGGLYHENMR
RPYFPVAVGKYYSYYCDEHFETPSGSYWDHIHCTQDGWSPAVPCLRKCY
FPYLENGYNQNHGRKFVQGKSIDVACHPGYALPKAQTTVTCMENGWSPT
PRCIRVSFTLGGGGSGLNDIFEAQKIEWHEGGGGSHHHHHH.

TABLE 6
Anti-C3 antibodies (CDR sequences)

| | | | |
|---|---|---|---|
| SEQ ID | Antibody | | |
| NO: | Name: | Descriptor: | Sequence: |

VH Family: IGHV-1, 3, 5, 7

| | | | |
|---|---|---|---|
| 582 | — | HCDR1 | X₁YX₂X₃X₄ |
| | | Consensus | (X₁ is G, S, N, D, T, or A; X₂ is Y, A, G, W, or T; X₃ is L, M, I, or |
| | | | V; X₄ is H, N, S, or G) |
| 583 | ATX-P-3369 | HCDR1 | GYYLH |
| 584 | ATX-P-3396 | HCDR1 | GYYMH |
| 585 | ATX-P-3413 | HCDR1 | GYYMH |
| 586 | ATX-P-3418 | HCDR1 | SYAMN |
| 587 | ATX-P-3419 | HCDR1 | SYGIS |
| 588 | ATX-P-3426 | HCDR1 | SYWIG |
| 589 | ATX-P-3428 | HCDR1 | GYGIS |
| 590 | ATX-P-3431 | HCDR1 | NYWIG |
| 591 | ATX-P-3432 | HCDR1 | DYYMH |
| 592 | ATX-P-3433 | HCDR1 | DYYMH |
| 593 | ATX-P-3435 | HCDR1 | NYGIS |
| 594 | ATX-P-3440 | HCDR1 | TYGIS |
| 595 | ATX-P-3452 | HCDR1 | GYYMN |
| 596 | ATX-P-3454 | HCDR1 | NYAVN |
| 597 | ATX-P-3458 | HCDR1 | GYYLH |
| 598 | ATX-P-3466 | HCDR1 | SYTMN |
| 599 | ATX-P-3469 | HCDR1 | AYAIH |
| 600 | ATX-P-3470 | HCDR1 | SYTMN |
| 601 | — | HCDR2 | (X₁ is W, I, or V; X₂ is N, S, or Y; X₃ is P, T, A, G, or Y; X₄ is N, |
| | | Consensus | Y, G, D, H, or A; X₅ is S, T, N, G, or D; X₆ is G, or S; X₇ is D, G, |
| | | | T, N, V, A, or H; X₈ is T, P, or K; X₉ is N, Y, T, H, R, I, D, or K; |
| | | | X₁₀ is Y, or A; X₁₁ is V, Q, A, or S; X₁₂ is Q, K, P, or D; X₁₃ is N, |
| | | | F, K, G, S, or Q; X₁₄ is F, Q, L, or V; X₁₅ is Q, G, T, or K) |
| 602 | ATX-P-3369 | HCDR2 | WINPNSGDTNYVQNFQG |
| 603 | ATX-P-3396 | HCDR2 | WINPNSGGTYAQKFOG |
| 604 | ATX-P-3413 | HCDR2 | WINPNSGGTTYAQKFQG |
| 605 | ATX-P-3418 | HCDR2 | WINTNTGTPTYAQGFTG |
| 606 | ATX-P-3419 | HCDR2 | WISAYNGNTHYAONLQG |
| 607 | ATX-P-3426 | HCDR2 | IIYPGDSDTRYSPSFQG |
| 608 | ATX-P-3428 | HCDR2 | WISAYNGNTHYAONLQG |
| 609 | ATX-P-3431 | HCDR2 | IIYPGDSDTIYSPSFQG |
| 610 | ATX-P-3432 | HCDR2 | WINPNSGVTYYAQKFQG |
| 611 | ATX-P-3433 | HCDR2 | WINPNNGGTDYAQKFQG |
| 612 | ATX-P-3435 | HCDR2 | WISAYNGNTHYAQKLOG |
| 613 | ATX-P-3440 | HCDR2 | WISAYSGNTNYAQKLOG |
| 614 | ATX-P-3452 | HCDR2 | WINPNSGATNYAQKFOG |
| 615 | ATX-P-3454 | HCDR2 | WINTNTGHPTYAQGFTG |
| 616 | ATX-P-3458 | HCDR2 | WINPHSGGTIYAQKFQD |
| 617 | ATX-P-3466 | HCDR2 | WINTATGTPTYAQGFTG |
| 618 | ATX-P-3469 | HCDR2 | VISYDGSNKKYADSVKG |
| 619 | ATX-P-3470 | HCDR2 | WINTNTGTPTYAQGFTG |
| 620 | ATX-P-3369 | HCDR3 | SYVDTSSSAFDI |
| 621 | ATX-P-3396 | HCDR3 | ERGDYDGDAFDI |
| 622 | ATX-P-3413 | HCDR3 | EAGGSGEYFQH |
| 623 | ATX-P-3418 | HCDR3 | HSTSEDY |
| 624 | ATX-P-3419 | HCDR3 | SIAALDF |
| 625 | ATX-P-3426 | HCDR3 | HDYFDIGGYFDY |
| 626 | ATX-P-3428 | HCDR3 | AGLGIDY |
| 627 | ATX-P-3431 | HCDR3 | GRGYSGYEGAFDI |
| 628 | ATX-P-3432 | HCDR3 | DRTFYFDY |
| 629 | ATX-P-3433 | HCDR3 | GVYDVVVPTAIVNWFDP |
| 630 | ATX-P-3435 | HCDR3 | AAAAVDY |
| 631 | ATX-P-3440 | HCDR3 | ERVSNWVFDF |
| 632 | ATX-P-3452 | HCDR3 | DGAKWNHEGYYFDY |
| 633 | ATX-P-3454 | HCDR3 | DSSSWGY |
| 634 | ATX-P-3458 | HCDR3 | WNSGDSYYYYMDV |
| 635 | ATX-P-3466 | HCDR3 | YSGSHSFDY |
| 636 | ATX-P-3469 | HCDR3 | GLPFDY |
| 637 | ATX-P-3470 | HCDR3 | YTGNYSEDY |

VH Family: IGHV3

| | | | |
|---|---|---|---|
| 638 | — | HCDR1 | X₁X₂X₃MX₄ |
| | | Consensus | (X₁ is D, N, S, or A; X₂ is Y, or A; X₃ is A, W, Y, S, C, D, or G; |
| | | | X₄ is H. S, or N) |
| 639 | ATX-P-3362 | HCDR1 | DYAMH |
| 640 | ATX-P-3366 | HCDR1 | NAWMS |
| 641 | ATX-P-3395 | HCDR1 | SYWMH |
| 642 | ATX-P-3397 | HCDR1 | NYYMN |
| 643 | ATX-P-3405 | HCDR1 | SYSMN |
| 644 | ATX-P-3407 | HCDR1 | SYCMN |
| 645 | ATX-P-3408 | HCDR1 | SYWMH |
| 646 | ATX-P-3410 | HCDR1 | AYDMH |
| 647 | ATX-P-3444 | HCDR1 | SYGMN |
| 648 | ATX-P-3463 | HCDR1 | SYAMH |
| 649 | ATX-P-3468 | HCDR1 | SYAMH |
| 650 | ATX-P-3471 | HCDR1 | SYAMS |
| 651 | — | HCDR2 | X₁IX₂X₃X₄X₅X₆X₇X₈X₉X₁₀X₁₁X₁₂X₁₃X₁₄X₁₅ |
| | | Consensus | (X₁ is G, R, N, S, H, T, V, or A; X₂ is S, K, N, or G; X₃ is W, S, |
| | | | Q, T, G, or Y; X₄ is N, K, D, S, A, or Y; X₅ is S, T, or G; X₆ is G, |
| | | | D, or S; X₇ is S, G, E, Y, T, or N; X₈ is I, G, T, K, or Y; X₉ is A, |
| | | | T, S, Y, or N; X₁₀ is Y, T, P, or F; X₁₁ is A, D, or V; X₁₂ is D, Y, |
| | | | S, or A; X₁₃ is S, A, or V; X₁₄ is V, A, or K; X₁₅ is K, P, Q, or G) |
| 652 | ATX-P-3362 | HCDR2 | GISWNSGSIAYADSVKG |
| 653 | ATX-P-3366 | HCDR2 | RIKSKTDGGTTDYAAPVKG |
| 654 | ATX-P-3395 | HCDR2 | RINSDGSSTSYADSVQG |
| 655 | ATX-P-3397 | HCDR2 | NIKQDGSEKYYVDSVKG |
| 656 | ATX-P-3405 | HCDR2 | SISSSSSYIYYADSVKG |
| 657 | ATX-P-3407 | HCDR2 | SINSDSSYIYYADSVKG |
| 658 | ATX-P-3408 | HCDR2 | HINSDGSSTNYADSVKG |
| 659 | ATX-P-3410 | HCDR2 | TIGTAGDTYYPDSVKG |
| 660 | ATX-P-3444 | HCDR2 | SISGYGSYIYYAASVKG |
| 661 | ATX-P-3463 | HCDR2 | VISYDGSNKYYADSVKG |
| 662 | ATX-P-3468 | HCDR2 | VISYDGSNKYYADSVKG |
| 663 | ATX-P-3471 | HCDR2 | AISGSGDSTYFADSVKG |
| 664 | ATX-P-3362 | HCDR3 | VAAAAGIPFFDY |
| 665 | ATX-P-3366 | HCDR3 | RYNSDFDY |
| 666 | ATX-P-3395 | HCDR3 | GOHLVODY |
| 667 | ATX-P-3397 | HCDR3 | DGYYNFDAFDI |
| 668 | ATX-P-3405 | HCDR3 | EGYDNWNSACDI |
| 669 | ATX-P-3407 | HCDR3 | EGYYNWNGGNDY |
| 670 | ATX-P-3408 | HCDR3 | EGYWNGDTFDI |
| 671 | ATX-P-3410 | HCDR3 | GGSSSWYDWYFDL |
| 672 | ATX-P-3444 | HCDR3 | ELGGIYYGVGYFDL |
| 673 | ATX-P-3463 | HCDR3 | RGGYSYGPFEH |
| 674 | ATX-P-3468 | HCDR3 | RGNWNYGPFHY |
| 675 | ATX-P-3471 | HCDR3 | ILGYCSGTSCYGLFDN |

VH Family: IGHV4-4

| | | | |
|---|---|---|---|
| 676 | — | HCDR1 | X₁X₂X₃WWX₄ |
| | | Consensus | (X₁ is I, S, R, N, or K; X₂ is N, D, S, or G; X₃ is N, S, T, D, Y, or |
| | | | H; X₄ is S, N, or T) |
| 677 | ATX-P-3360 | HCDR1 | INNWWS |
| 678 | ATX-P-3361 | HCDR1 | SNNWWS |
| 679 | ATX-P-3363 | HCDR1 | RDNWWS |
| 680 | ATX-P-3365 | HCDR1 | SNNWWS |
| 681 | ATX-P-3373 | HCDR1 | NDNWWS |
| 682 | ATX-P-3378 | HCDR1 | NDNWWS |
| 683 | ATX-P-3382 | HCDR1 | SNSWWN |
| 684 | ATX-P-3383 | HCDR1 | KDNWWT |
| 685 | ATX-P-3384 | HCDR1 | SDNWWN |
| 686 | ATX-P-3386 | HCDR1 | NDNWWS |
| 687 | ATX-P-3387 | HCDR1 | NDNWWN |
| 688 | ATX-P-3389 | HCDR1 | SSNWWS |
| 689 | ATX-P-3391 | HCDR1 | NDNWWS |
| 690 | ATX-P-3392 | HCDR1 | NDNWWS |
| 691 | ATX-P-3412 | HCDR1 | SSTWWS |
| 692 | ATX-P-3421 | HCDR1 | SNNWWS |
| 693 | ATX-P-3430 | HCDR1 | SSDWWS |
| 694 | ATX-P-3437 | HCDR1 | SNNWWS |
| 695 | ATX-P-3438 | HCDR1 | NDYWWN |
| 696 | ATX-P-3441 | HCDR1 | SGHWWS |
| 697 | ATX-P-3442 | HCDR1 | SSDWWS |
| 698 | ATX-P-3453 | HCDR1 | SNNWWS |
| 699 | ATX-P-3455 | HCDR1 | SDNWWS |
| 700 | ATX-P-3456 | HCDR1 | SNNWWS |
| 701 | ATX-P-3459 | HCDR1 | SDNWWN |
| 702 | ATX-P-3462 | HCDR1 | SNNWWS |
| 703 | ATX-P-3467 | HCDR1 | SDYWWS |
| 704 | ATX-P-3472 | HCDR1 | NDNWWS |
| 705 | — | HCDR2 | X₁IX₂X₃SX₄X₅X₆X₇X₈NPSLX₉S |
| | | Consensus | (X₁ is K, Q, R, E, or D; X₂ is Y or F; X₃ is H or Q; X₄ is G, E, or |
| | | | R; X₅ is N, I, S, H, F, or T; X₆ is T or I; X₇ is D, N, or M; X₈ is Y |
| | | | or H; X₉ is K or R) |
| 706 | ATX-P-3360 | HCDR2 | KIYHSGNTDYNPSLKS |
| 707 | ATX-P-3361 | HCDR2 | QIYHSGITNYNPSLKS |
| 708 | ATX-P-3363 | HCDR2 | QIYQSGSTNYNPSLKS |
| 709 | ATX-P-3365 | HCDR2 | QIYHSGHTNYNPSLKS |
| 710 | ATX-P-3373 | HCDR2 | KIYHSEFTNYNPSLKS |
| 711 | ATX-P-3378 | HCDR2 | KIYHSEFTNYNPSLKS |
| 712 | ATX-P-3382 | HCDR2 | QIFHSGHTNYNPSLKS |
| 713 | ATX-P-3383 | HCDR2 | KIYHSGSTNYNPSLKS |
| 714 | ATX-P-3384 | HCDR2 | KIYHSGNTDYNPSLKS |
| 715 | ATX-P-3386 | HCDR2 | KIYHSGFTNYNPSLKS |
| 716 | ATX-P-3387 | HCDR2 | KIYHSGFTDYNPSLKS |
| 717 | ATX-P-3389 | HCDR2 | RIYHSGHTNYNPSLKS |
| 718 | ATX-P-3391 | HCDR2 | KIYHSEFTNYNPSLKS |
| 719 | ATX-P-3392 | HCDR2 | KIYHSEFTNYNPSLKS |
| 720 | ATX-P-3412 | HCDR2 | EIYHSGSTNYNPSLKS |
| 721 | ATX-P-3421 | HCDR2 | QIYHSENTNYNPSLKS |
| 722 | ATX-P-3430 | HCDR2 | EIYHSGSTNYNPSLKS |
| 723 | ATX-P-3437 | HCDR2 | EIYHSGNINYNPSLKS |
| 724 | ATX-P-3438 | HCDR2 | EIYHSGHTNYNPSLKS |
| 725 | ATX-P-344] | HCDR2 | EIYHSGNTNYNPSLKS |
| 726 | ATX-P-3442 | HCDR2 | EIYHSRNTNYNPSLKS |
| 727 | ATX-P-3453 | HCDR2 | RIYHSGTTNYNPSLES |
| 728 | ATX-P-3455 | HCDR2 | EIYHSENTNYNPSLKS |
| 729 | ATX-P-3456 | HCDR2 | DIYHSGNTNYNPSLKS |
| 730 | ATX-P-3459 | HCDR2 | KIYHSGNTNYNPSLKS |
| 731 | ATX-P-3462 | HCDR2 | KIYQSGSTMYNPSLKS |
| 732 | ATX-P-3467 | HCDR2 | KIYHSGNTNHNPSLKS |
| 733 | ATX-P-3472 | HCDR2 | KIYHSEFTNYNPSLRS |
| 734 | ATX-P-3360 | HCDR3 | GSDYDFWSGYLDY |
| 735 | ATX-P-3361 | HCDR3 | AQWELRAGWFDP |
| 736 | ATX-P-3363 | HCDR3 | VYDWNYGGHEDY |
| 737 | ATX-P-3365 | HCDR3 | GATEILGYFDL |
| 738 | ATX-P-3373 | HCDR3 | EYHNFMDV |
| 739 | ATX-P-3378 | HCDR3 | EYENFMDV |
| 740 | ATX-P-3382 | HCDR3 | GGEYENSANWFDP |
| 741 | ATX-P-3383 | HCDR3 | GGDIAMEGDWFAP |
| 742 | ATX-P-3384 | HCDR3 | GGTETTGYMDV |
| 743 | ATX-P-3386 | HCDR3 | GGDNFMEGEGLGT |
| 744 | ATX-P-3387 | HCDR3 | GNHNYLWIGYLGT |
| 745 | ATX-P-3389 | HCDR3 | GRDLTGYFDY |
| 746 | ATX-P-3391 | HCDR3 | GVDFWSGSHMDV |
| 747 | ATX-P-3392 | HCDR3 | EGLELPHMDV |
| 748 | ATX-P-3412 | HCDR3 | SLYNWNDAAFDI |
| 749 | ATX-P-3421 | HCDR3 | VDDWNYGGHFDY |
| 750 | ATX-P-3430 | HCDR3 | GGHIGYYYMDV |
| 751 | ATX-P-3437 | HCDR3 | GGEYGDPVGYFDL |
| 752 | ATX-P-3438 | HCDR3 | GGTEITGYMDV |
| 753 | ATX-P-3441 | HCDR3 | GHYGDYVVYFDY |
| 754 | ATX-P-3442 | HCDR3 | GGVVADYYMDV |
| 755 | ATX-P-3453 | HCDR3 | GEEWELRGGVEDI |
| 756 | ATX-P-3455 | HCDR3 | GRELTGLFDY |
| 757 | ATX-P-3456 | HCDR3 | GGIAVTAYFDY |
| 758 | ATX-P-3459 | HCDR3 | GVDFWSGSHMDV |
| 759 | ATX-P-3462 | HCDR3 | DKPPLLGAFDY |
| 760 | ATX-P-3467 | HCDR3 | EGLELPHMDV |
| 761 | ATX-P-3472 | HCDR3 | EYQNYMDV |

VH Family: IGHV4-5

| | | | |
|---|---|---|---|
| 762 | — | HCDR1 | X₁YYX₂X₃ |
| | | Consensus | (X₁ is S, or D; X₂ is W, or L; X₃ is S, or T) |
| 763 | ATX-P-3370 | HCDR1 | SYYWS |
| 764 | ATX-P-3371 | HCDR1 | SYYWS |
| 765 | ATX-P-3376 | HCDR1 | SYYWS |
| 766 | ATX-P-3420 | HCDR1 | DYYLT |
| 767 | ATX-P-3446 | HCDR1 | SYYWS |
| 768 | ATX-P-3449 | HCDR1 | SYYWS |
| 769 | ATX-P-3451 | HCDR | DYYWT |
| 770 | — | HCDR2 | X₁X₂X₃X₄X₅GX₆TX₇YNPSLKS |
| | | Consensus | (X₁ is Y or E; X₂ is V or I; X₃ is Y or N; X₄ is Y or H; X₅ is S or I; |
| | | | X₆ is Y, F, or S; X₇ is N or K) |
| 771 | ATX-P-3370 | HCDR2 | YVYYSGYTKYNPSLKS |
| 772 | ATX-P-3371 | HCDR2 | YIYYSGFTKYNPSLNS |
| 773 | ATX-P-3376 | HCDR2 | YIYYSGFTKYNPSLKS |
| 774 | ATX-P-3420 | HCDR2 | EINHSGSTNYNPSLKS |
| 775 | ATX-P-3446 | HCDR2 | YVYYSGYTKYNPSLKS |
| 776 | ATX-P-3449 | HCDR2 | YIYYSGFTKYNPSLKS |
| 777 | ATX-P-3451 | HCDR2 | EVNHIGSTNYNPSLKS |
| 778 | ATX-P-3370 | HCDR3 | VESKWNYGPFDI |
| 779 | ATX-P-3371 | HCDR3 | REPRGRYNWNYGPFDI |
| 780 | ATX-P-3376 | HCDR3 | HPRGYNYGPFDY |
| 781 | ATX-P-3420 | HCDR3 | GSEDYYYYMDV |
| 782 | ATX-P-3446 | HCDR3 | VESKWNYGPFAI |
| 783 | ATX-P-3449 | HCDR3 | VDSKWNYGSFHI |
| 784 | ATX-P-3451 | HCDR3 | YSTSWYGWFDP |

VH Family: IGHV4-6

| | | | |
|---|---|---|---|
| 785 | — | HCDR1 | X₁X₂X₃YXAWX₅ |
| | | Consensus | (X₁ is S or N; X₂ is G or N; X₃ is S, K, R, N, or G; X₄ is Y, F, or |
| | | | N; X₅ is S or N) |
| 786 | ATX-P-3367 | HCDR1 | SGSYYWS |
| 787 | ATX-P-3375 | HCDR1 | SGKYFWS |
| 788 | ATX-P-3377 | HCDR1 | SGRYYWS |
| 789 | ATX-P-3436 | HCDR1 | SGSYYWS |
| 790 | ATX-P-3447 | HCDR1 | SGNYYWS |
| 791 | ATX-P-3448 | HCDR1 | SGGYNWN |
| 792 | ATX-P-3450 | HCDR1 | NNRYYWS |
| 793 | — | HCDR2 | YIX₁X₂SGX₃TX₄YNPSLKX₅ |
| | | Consensus | (X₁ is Y or N; X₂ is K, Y, or S; X₃ is F, S, Y, or N; X₄ is N, K, or |
| | | | Y; X₅ is S or N) |
| 794 | ATX-P-3367 | HCDR2 | YIYKSGFTNYNPSLKS |
| 795 | ATX-P-3375 | HCDR2 | YIYYSGSTNYNPSLKN |
| 796 | ATX-P-3377 | HCDR2 | YIYSSGYTNYNPSLKS |
| 797 | ATX-P-3436 | HCDR2 | YIYYSGSTKYNPSLKS |
| 798 | ATX-P-3447 | HCDR2 | YIYSSGSTNYNPSLKS |
| 799 | ATX-P-3448 | HCDR2 | YINYSGNTYYNPSLKS |
| 800 | ATX-P-3450 | HCDR2 | YIYYSGSTNYNPSLKS |
| 801 | ATX-P-3367 | HCDR3 | GGRGYSYGPFDY |
| 802 | ATX-P-3375 | HCDR3 | GGHNWNYAAFDI |
| 803 | ATX-P-3377 | HCDR3 | SRGYRYGPEDY |
| 804 | ATX-P-3436 | HCDR3 | DKGHWNYGAFDI |
| 805 | ATX-P-3447 | HCDR3 | SPRGYSYGPFAY |
| 806 | ATX-P-3448 | HCDR3 | GPLDTAMAPFDY |
| 807 | ATX-P-3450 | HCDR3 | DRPSRYKWNYDAFAI |

VH Family: IGHV2

| | | | |
|---|---|---|---|
| 808 | — | HCDR1 | TSGVGVG |
| | | Consensus | |
| 809 | ATX-P-3406 | HCDR1 | TSGVGVG |
| 810 | ATX-P-3414 | HCDR1 | TSGVGVG |
| 811 | — | HCDR2 | LIYWNX₁X₂X₃X₄YSPSLX₅S |
| | | Consensus | (X₁ is G or D; X₂ is D or V; X₃ is E or K; X₄ is R or F; |
| | | | X₅ is K or Q) |
| 812 | ATX-P-3406 | HCDR2 | LIYWNGDERYSPSLKS |
| 813 | ATX-P-3414 | HCDR2 | LIYWNDVKFYSPSLOS |
| 814 | — | HCDR3 | X₁X₂NWX₃YX₄AFDI |
| | | Consensus | (X₁ is S or G; X₂ is P or D; X₃ is G or N; X₄ is E or R) |
| 815 | ATX-P-3406 | HCDR3 | SPNWGYEAFDI |
| 816 | ATX-P-3414 | HCDR3 | GDNWNYRAFDI |

VL Family: IGKV-1, 6

| | | | |
|---|---|---|---|
| 817 | — | LCDR1 | X₁ASQX₂X₃X₄X₅X₆X₇X₈ |
| | | Consensus | (X₁ is R or Q; X₂ is D, N, T, G, or S; X₃ is V or I; X₄ is S, I, R, G, |
| | | | T, or K; X₅ is T, N, R, or S; X₆ is A, W, F, D, Y, N, or C; X₇ is V |
| | | | or L; X₈ is A, N. G, or H) |
| 818 | ATX-P-1899 | LCDR1 | RASQDVSTAVA |
| 819 | ATX-P-3395 | LCDR1 | RASONIITWLA |
| 820 | ATX-P-3396 | LCDR1 | RASQNIITWLA |
| 821 | ATX-P-3397 | LCDR1 | RASQNIITWLA |
| 822 | ATX-P-3405 | LCDR1 | RASONIITWLA |
| 823 | ATX-P-3406 | LCDR1 | RASONIITWLA |
| 824 | ATX-P-3407 | LCDR1 | RASQNIITWLA |
| 825 | ATX-P-3408 | LCDR1 | RASQNIITWLA |
| 826 | ATX-P-3410 | LCDR1 | RASQNIITWLA |
| 827 | ATX-P-3412 | LCDR1 | RASQNIITWLA |
| 828 | ATX-P-3413 | LCDR1 | RASONIITWLA |
| 829 | ATX-P-3414 | LCDR1 | RASONIITWLA |
| 830 | ATX-P-3418 | LCDR1 | RASOTIRNFLN |
| 831 | ATX-P-3419 | LCDR1 | RASQGIRNDLG |
| 832 | ATX-P-3420 | LCDR1 | RASQSISRYLN |
| 833 | ATX-P-3428 | LCDR1 | RASQGIRNDLG |
| 834 | ATX-P-3430 | LCDR1 | RASQSISNYLN |
| 835 | ATX-P-3431 | LCDR1 | RASQSIGSNLH |
| 836 | ATX-P-3432 | LCDR1 | RASQGISSYLA |
| 837 | ATX-P-3433 | LCDR1 | RASQSITSYLN |
| 838 | ATX-P-3435 | LCDR1 | RASQGIRNDLG |
| 839 | ATX-P-3441 | LCDR1 | RASQSISSYLN |
| 840 | ATX-P-3442 | LCDR1 | RASQTIRSYLN |
| 841 | ATX-P-3448 | LCDR1 | QASQDISNYLN |
| 842 | ATX-P-3451 | LCDR1 | RASODISNYLN |
| 843 | ATX-P-3454 | LCDR1 | QASQDIKNCLN |
| 844 | ATX-P-3458 | LCDR1 | RASQGISSWLA |
| 845 | ATX-P-3462 | LCDR1 | RASOSISSYLN |
| 846 | ATX-P-3466 | LCDR1 | RASQGISNYLA |
| 847 | ATX-P-3469 | LCDR1 | RASQGISNYLA |
| 848 | ATX-P-3470 | LCDR1 | RASQGISNYLA |
| 849 | — | LCDR2 | X₁ASX₂X₃X₄X₅ |
| | | Consensus | (X₁ is S, K, A, Y, T, D, or V; X₂ is F, S, N, Q, or T; X₃ is L, S, or |
| | | | F; X₄ is Y, E, Q, or F; X₅ is S or T) |
| 850 | ATX-P-1899 | LCDR2 | SASFLYS |
| 851 | ATX-P-3395 | LCDR2 | KASSLES |
| 852 | ATX-P-3396 | LCDR2 | KASSLES |
| 853 | ATX-P-3397 | LCDR2 | KASSLES |
| 854 | ATX-P-3405 | LCDR2 | KASSLES |
| 855 | ATX-P-3406 | LCDR2 | KASSLES |
| 856 | ATX-P-3407 | LCDR2 | KASSLES |
| 857 | ATX-P-3408 | LCDR2 | KASSLES |
| 858 | ATX-P-3410 | LCDR2 | KASSLES |
| 859 | ATX-P-3412 | LCDR2 | KASSLES |
| 860 | ATX-P-3413 | LCDR2 | KASSLES |
| 861 | ATX-P-3414 | LCDR2 | KASSLES |
| 862 | ATX-P-3418 | LCDR2 | AASSLQS |
| 863 | ATX-P-3419 | LCDR2 | AASSLQS |
| 864 | ATX-P-3420 | LCDR2 | AASSLQS |
| 865 | ATX-P-3428 | LCDR2 | AASNLOS |
| 866 | ATX-P-3430 | LCDR2 | AASSLQS |
| 867 | ATX-P-3431 | LCDR2 | YASOSFS |
| 868 | ATX-P-3432 | LCDR2 | TASTLOS |
| 869 | ATX-P-3433 | LCDR2 | AASSLQS |
| 870 | ATX-P-3435 | LCDR2 | AASSLQS |
| 871 | ATX-P-3441 | LCDR2 | AASSFOS |
| 872 | ATX-P-3442 | LCDR2 | AASSLQS |
| 873 | ATX-P-3448 | LCDR2 | DASNLET |
| 874 | ATX-P-3451 | LCDR2 | AASSLES |
| 875 | ATX-P-3454 | LCDR2 | DASNLET |
| 876 | ATX-P-3458 | LCDR2 | AASSLQS |
| 877 | ATX-P-3462 | LCDR2 | AASSLQS |
| 878 | ATX-P-3466 | LCDR2 | VASTLOS |
| 879 | ATX-P-3469 | LCDR2 | AASTLOS |
| 880 | ATX-P-3470 | LCDR2 | AASTLOS |
| 881 | ATX-P-1899 | LCDR3 | QQSYATLPT |
| 882 | ATX-P-3395 | LCDR3 | QOYSSYSYT |
| 883 | ATX-P-3396 | LCDR3 | QOYSSYSYT |
| 884 | ATX-P-3397 | LCDR3 | QQYSSYSYT |
| 885 | ATX-P-3405 | LCDR3 | QQYSSYSYT |
| 886 | ATX-P-3406 | LCDR3 | QQYSSYSYT |
| 887 | ATX-P-3407 | LCDR3 | QQYSSYSYT |
| 888 | ATX-P-3408 | LCDR3 | QQYSSYSYT |
| 889 | ATX-P-3410 | LCDR3 | QQYSSYSYT |
| 890 | ATX-P-3412 | LCDR3 | QQYSSYSYT |
| 891 | ATX-P-3413 | LCDR3 | QQYSSYSYT |
| 892 | ATX-P-3414 | LCDR3 | QQYSSYSYT |
| 893 | ATX-P-3418 | LCDR3 | QQTYSIPIT |
| 894 | ATX-P-3419 | LCDR3 | LODYNYPWT |
| 895 | ATX-P-3420 | LCDR3 | QQSYSSPIT |
| 896 | ATX-P-3428 | LCDR3 | LODYNWPWT |
| 897 | ATX-P-3430 | LCDR3 | QQSYSSPLT |
| 898 | ATX-P-3431 | LCDR3 | HOSSSLPRT |
| 899 | ATX-P-3432 | LCDR3 | QQLNGYPPT |
| 900 | ATX-P-3433 | LCDR3 | QQSYSTPLT |
| 901 | ATX-P-3435 | LCDR3 | LODYNWPYT |
| 902 | ATX-P-3441 | LCDR3 | QQSYSNPYT |
| 903 | ATX-P-3442 | LCDR3 | QQSYTTPLT |
| 904 | ATX-P-3448 | LCDR3 | QQANSFPLT |
| 905 | ATX-P-3451 | LCDR3 | QQSYSTPT |
| 906 | ATX-P-3454 | LCDR3 | QQFDNLPLT |
| 907 | ATX-P-3458 | LCDR3 | QQANSFPLT |
| 908 | ATX-P-3462 | LCDR3 | QQSYSTPYT |
| 909 | ATX-P-3466 | LCDR3 | QNYISAPLT |
| 910 | ATX-P-3469 | LCDR3 | QKYISAPLT |
| 911 | ATX-P-3470 | LCDR3 | QDYTSAPLT |

VL Family: IGKV2

| | | | |
|---|---|---|---|
| 912 | — | LCDR1 | RSSQX₁LLHX₂NGYNYLD |
| | | Consensus | (X₁ is S or I; X₂ is S or T) |
| 913 | ATX-P-3426 | LCDR1 | RSSQSLLHSNGYNYLD |
| 914 | ATX-P-3452 | LCDR1 | RSSQILLHINGYNYLD |
| 915 | — | LCDR2 | LGSX1RAS |
| | | Consensus | (X₁ is N or Y) |
| 916 | ATX-P-3426 | LCDR2 | LGSNRAS |
| 917 | ATX-P-3452 | LCDR2 | LGSYRAS |
| 918 | | LCDR3 | MQX₁LQTPX₂T |
| | | Consensus | (X₁ is T or A; X₂ is P or L) |
| 919 | ATX-P-3426 | LCDR3 | MOTLOTPPT |
| 920 | ATX-P-3452 | LCDR3 | MQALQTPLT |

VL Family: IGKV3

| | | | |
|---|---|---|---|
| 921 | — | LCDR1 | RASQSX₁X₂X₃X₄X₅X₆ |
| | | Consensus | (X₁ is V or I; X₂ is S or R; X₃ is S, D, or R; X₄ is Y or S; X₅ is L or |
| | | | Y; X₆ is A, F, or L) |
| 922 | ATX-P-3436 | LCDR1 | RASQSVSSYLA |
| 923 | ATX-P-3440 | LCDR1 | RASQSVRSSYFA |
| 924 | ATX-P-3444 | LCDR1 | RASOSISSSYLA |
| 925 | ATX-P-3446 | LCDR | RASQSVRSSYLA |
| 926 | ATX-P-3447 | LCDR1 | RASOSVSSSYLA |
| 927 | ATX-P-3449 | LCDR1 | RASQSVSSSYLA |
| 928 | ATX-P-3468 | LCDR1 | RASOSVSDSYLA |
| 929 | ATX-P-3471 | LCDR1 | RASOSVSRSYLA |
| 930 | — | LCDR2 | X₁ASX₂RAT |
| | | Consensus | (X₁ is D or G; X₂ is S or N) |
| 931 | ATX-P-3436 | LCDR2 | DASNRAT |
| 932 | ATX-P-3440 | LCDR2 | GASSRAT |
| 933 | ATX-P-3444 | LCDR2 | GASSRAT |
| 934 | ATX-P-3446 | LCDR2 | GASSRAT |
| 935 | ATX-P-3447 | LCDR2 | GASSRAT |
| 936 | ATX-P-3449 | LCDR2 | GASSRAT |
| 937 | ATX-P-3468 | LCDR2 | GASSRAT |
| 938 | ATX-P-3471 | LCDR2 | GASSRAT |
| 939 | — | LCDR3 | QQX₁X₂X₃X₄PX₅T |
| | | Consensus | (X₁ is R or Y; X₂ is S, G, or D; X₃ is N or S; X₄ is W or S; X₅ is L, |
| | | | R, I, Y, F, or W) |
| 940 | ATX-P-3436 | LCDR3 | QQRSNWPLT |
| 941 | ATX-P-3440 | LCDR3 | QQYGSSPRT |
| 942 | ATX-P-3444 | LCDR3 | QQYGSSPIT |
| 943 | ATX-P-3446 | LCDR3 | QQYGSSPYT |
| 944 | ATX-P-3447 | LCDR3 | QQYGSSPFT |
| 945 | ATX-P-3449 | LCDR3 | QQYGSSPYT |
| 946 | ATX-P-3468 | LCDR3 | QQYDSSPFT |
| 947 | ATX-P-3471 | LCDR3 | QQYGSSPWT |

VL Family: IGKV4

| | | | |
|---|---|---|---|
| 948 | — | LCDR1 | KSSQX₁X₂LYX₃SNX₄KNYLA |
| | | Consensus | (X₁ is S or T; X₂ is V or I; X₃ is R or S; X₄ is N or I) |
| 949 | ATX-P-3360 | LCDR1 | KSSQSVLYSSNNKNYLA |
| 950 | ATX-P-3361 | LCDR1 | KSSQSVLYSSNNKNYLA |
| 951 | ATX-P-3362 | LCDR1 | KSSQSVLYSSNNKNYLA |
| 952 | ATX-P-3363 | LCDR1 | KSSQSVLYSSNNKNYLA |
| 953 | ATX-P-3365 | LCDR1 | KSSQSVLYSSNNKNYLA |
| 954 | ATX-P-3366 | LCDR1 | KSSQSVLYSSNNKNYLA |
| 955 | ATX-P-3367 | LCDR1 | KSSQSVLYSSNNKNYLA |
| 956 | ATX-P-3369 | LCDR1 | KSSQSVLYSSNNKNYLA |
| 957 | ATX-P-3370 | LCDR1 | KSSQSVLYSSNNKNYLA |
| 958 | ATX-P-3371 | LCDR1 | KSSOSVLYSSNNKNYLA |
| 959 | ATX-P-3373 | LCDR1 | KSSQSVLYSSNNKNYLA |
| 960 | ATX-P-3375 | LCDR1 | KSSOSVLYSSNNKNYLA |
| 961 | ATX-P-3376 | LCDR1 | KSSQSVLYSSNNKNYLA |
| 962 | ATX-P-3377 | LCDR1 | KSSQSVLYSSNNKNYLA |
| 963 | ATX-P-3378 | LCDR1 | KSSQSVLYSSNNKNYLA |
| 964 | ATX-P-3382 | LCDR1 | KSSQSVLYSSNNKNYLA |
| 965 | ATX-P-3383 | LCDR1 | KSSQSVLYSSNNKNYLA |
| 966 | ATX-P-3384 | LCDR1 | KSSQSVLYSSNNKNYLA |
| 967 | ATX-P-3386 | LCDR1 | KSSQSVLYSSNNKNYLA |
| 968 | ATX-P-3387 | LCDR1 | KSSQSVLYSSNNKNYLA |
| 969 | ATX-P-3389 | LCDR1 | KSSQSVLYSSNNKNYLA |
| 970 | ATX-P-3391 | LCDR | KSSQSVLYSSNNKNYLA |
| 971 | ATX-P-3392 | LCDR | KSSQSVLYSSNNKNYLA |
| 972 | ATX-P-3421 | LCDR1 | KSSQTVLYRSNNKNYLA |
| 973 | ATX-P-3437 | LCDR1 | KSSQSVLYSSNNKNYLA |
| 974 | ATX-P-3438 | LCDR | KSSQSVLYSSNNKNYLA |
| 975 | ATX-P-3450 | LCDR1 | KSSQSVLYSSNNKNYLA |
| 976 | ATX-P-3453 | LCDR1 | KSSQSVLYRSNNKNYLA |
| 977 | ATX-P-3455 | LCDR1 | KSSQTILYRSNIKNYLA |
| 978 | ATX-P-3456 | LCDR1 | KSSQSVLYSSNNKNYLA |
| 979 | ATX-P-3459 | LCDR1 | KSSQSVLYSSNNKNYLA |
| 980 | ATX-P-3463 | LCDR1 | KSSQSVLYSSNNKNYLA |
| 981 | ATX-P-3467 | LCDR1 | KSSQSVLYSSNNKNYLA |
| 982 | ATX-P-3472 | LCDR | KSSQSVLYSSNNKNYLA |
| 983 | — | LCDR2 | WASX₁RES |
| | | Consensus | (X₁ is N or T) |
| 984 | ATX-P-3360 | LCDR2 | WASNRES |
| 985 | ATX-P-3361 | LCDR2 | WASNRES |
| 986 | ATX-P-3362 | LCDR2 | WASNRES |
| 987 | ATX-P-3363 | LCDR2 | WASNRES |
| 988 | ATX-P-3365 | LCDR2 | WASNRES |
| 989 | ATX-P-3366 | LCDR2 | WASNRES |
| 990 | ATX-P-3367 | LCDR2 | WASNRES |
| 991 | ATX-P-3369 | LCDR2 | WASNRES |
| 992 | ATX-P-3370 | LCDR2 | WASNRES |
| 993 | ATX-P-3371 | LCDR2 | WASNRES |
| 994 | ATX-P-3373 | LCDR2 | WASNRES |
| 995 | ATX-P-3375 | LCDR2 | WASNRES |
| 996 | ATX-P-3376 | LCDR2 | WASNRES |
| 997 | ATX-P-3377 | LCDR2 | WASNRES |
| 998 | ATX-P-3378 | LCDR2 | WASNRES |
| 999 | ATX-P-3382 | LCDR2 | WASNRES |
| 1000 | ATX-P-3383 | LCDR2 | WASNRES |
| 1001 | ATX-P-3384 | LCDR2 | WASNRES |
| 1002 | ATX-P-3386 | LCDR2 | WASNRES |
| 1003 | ATX-P-3387 | LCDR2 | WASNRES |
| 1004 | ATX-P-3389 | LCDR2 | WASNRES |
| 1005 | ATX-P-3391 | LCDR2 | WASNRES |
| 1006 | ATX-P-3392 | LCDR2 | WASNRES |
| 1007 | ATX-P-3421 | LCDR2 | WASTRES |
| 1008 | ATX-P-3437 | LCDR2 | WASTRES |
| 1009 | ATX-P-3438 | LCDR2 | WASTRES |
| 1010 | ATX-P-3450 | LCDR2 | WASTRES |
| 1011 | ATX-P-3453 | LCDR2 | WASTRES |
| 1012 | ATX-P-3455 | LCDR2 | WASTRES |
| 1013 | ATX-P-3456 | LCDR2 | WASTRES |
| 1014 | ATX-P-3459 | LCDR2 | WASTRES |
| 1015 | ATX-P-3463 | LCDR2 | WASTRES |
| 1016 | ATX-P-3467 | LCDR2 | WASTRES |
| 1017 | ATX-P-3472 | LCDR2 | WASTRES |
| 1018 | — | LCDR3 | QQX₁X₂X₃TX₄X₅ |
| | | Consensus | (X₁ is Y or F; X₂ is Y or F; X₃ is S, G, or D; X₄ is P or R; X₅ is Y, |
| | | | W, R, T, or P) |
| 1019 | ATX-P-3360 | LCDR3 | QQYYSTPYT |
| 1020 | ATX-P-336] | LCDR3 | QQYYSTPYT |
| 1021 | ATX-P-3362 | LCDR3 | QQYYSTPYT |
| 1022 | ATX-P-3363 | LCDR3 | QQYYSTPYT |
| 1023 | ATX-P-3365 | LCDR3 | QQYYSTPYT |
| 1024 | ATX-P-3366 | LCDR3 | QQYYSTPYT |
| 1025 | ATX-P-3367 | LCDR3 | QQYYSTPYT |
| 1026 | ATX-P-3369 | LCDR3 | QQYYSTPYT |
| 1027 | ATX-P-3370 | LCDR3 | QQYYSTPYT |
| 1028 | ATX-P-3371 | LCDR3 | QQYYSTPYT |
| 1029 | ATX-P-3373 | LCDR3 | QQYYSTPYT |
| 1030 | ATX-P-3375 | LCDR3 | QQYYSTPYT |
| 1031 | ATX-P-3376 | LCDR3 | QQYYSTPYT |
| 1032 | ATX-P-3377 | LCDR3 | QQYYSTPYT |
| 1033 | ATX-P-3378 | LCDR3 | QQYYSTPYT |
| 1034 | ATX-P-3382 | LCDR3 | QQYYSTPYT |
| 1035 | ATX-P-3383 | LCDR3 | QQYYSTPYT |
| 1036 | ATX-P-3384 | LCDR3 | QQYYSTPYT |
| 1037 | ATX-P-3386 | LCDR3 | QQYYSTPYT |
| 1038 | ATX-P-3387 | LCDR3 | QOYYSTPYT |
| 1039 | ATX-P-3389 | LCDR3 | QQYYSTPYT |
| 1040 | ATX-P-3391 | LCDR3 | QQYYSTPYT |
| 1041 | ATX-P-3392 | LCDR3 | QQYYSTPYT |
| 1042 | ATX-P-3421 | LCDR3 | QQYYSTPWT |
| 1043 | ATX-P-3437 | LCDR3 | QQYFGTPRT |
| 1044 | ATX-P-3438 | LCDR3 | QQYYSTPRT |
| 1045 | ATX-P-3450 | LCDR3 | QQYYSTPWT |
| 1046 | ATX-P-3453 | LCDR3 | QQYYGTPRT |
| 1047 | ATX-P-3455 | LCDR3 | QQYYSTRT |
| 1048 | ATX-P-3456 | LCDR3 | QQYYSTPRT |
| 1049 | ATX-P-3459 | LCDR3 | QQYYSTPPT |
| 1050 | ATX-P-3463 | LCDR3 | QQFYDTPRT |
| 1051 | ATX-P-3467 | LCDR3 | QQYYSTPWT |
| 1052 | ATX-P-3472 | LCDR3 | QQYYSTPYT |

TABLE 7
Anti-C3 antibodies (VH sequences)

| SEQ | | | |
|---|---|---|---|
| ID | Antibody | | |
| NO: | Name: | Descriptor: | Sequence: |
| 1053 | ATX-P-3360 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | CATTAATAACTGGTGGAGTTGGGTCCGCCAGCCCCCAGGGAAGG |
| | | | GGCTGGAGTGGCTTGGAAAAATCTATCATAGTGGGAACACCGAC |
| | | | TACAACCCGTCCCTCAAGAGTCGAGTCACCATATCAGTAGACAAG |
| | | | TCCAAGAATCAGTTCTCCCTGAGGCTGAGTTCTGTGACCGCCGCG |
| | | | GACACGGCCGTATATTATTGTGCGAGCGGAAGCGATTACGATTTT |
| | | | TGGAGTGGTTATTTGGACTACTGGGGCCAGGGAACCCTGGTCACC |
| | | | GTCTCCTCA |
| 1054 | ATX-P-3362 | VH nt | GAAGTGCAGCTGGTGGAGTCTGGGGGAGGCTTGGTACAGCCTGG |
| | | | CAGGTCCCTGAGACTCTCCTGTGCAGCCTCTGGATTCACCTTTGAT |
| | | | GATTATGCCATGCACTGGGTCCGGCAAGCTCCAGGGAAGGGCCT |
| | | | GGAGTGGGTCTCAGGTATTAGTTGGAATAGTGGTAGCATAGCCTA |
| | | | TGCGGACTCTGTGAAGGGCCGATTCACCATCTCCAGAGACAACGC |
| | | | CAAGAACTCCCTGTATCTGCAAATGAACAGTCTGAGAGTTGAGG |
| | | | ACACGGCCTTTTTTTACTGTGCAAGAGTCGCAGCAGCAGCTGGTA |
| | | | TCCCTTTCTTTGACTACTGGGGCCAGGGAACCCTGGTCACCGTCT |
| | | | CCTCA |
| 1055 | ATX-P-3363 | VH nt | CAGGTGCAGCTGCAGGAGTCGGGCCCAGGACTGGTGAAGCCTTC |
| | | | TGGGACCCTGACCCTCACCTGCGCTGTCTCTGGTGGCTCCATCAA |
| | | | CAGGGATAACTGGTGGAGTTGGGTCCGCCAGCCCCCGGGGAAGG |
| | | | GGCTGGAGTGGATTGGACAAATCTATCAGAGTGGGAGCACCAAC |
| | | | TACAACCCGTCCCTCAAGAGTCGGGTCACCATATCAGTCGACAAG |
| | | | TCCAAGAACCAGTTCTCCCTGAAGCTGAGTTCTGTGACCGCCGCG |
| | | | GACACGGCCGTATATTACTGTGCGAGAGTATATGACTGGAACTAC |
| | | | GGGGGTCACTTTGACTACTGGGGCCAGGGAACCCTGGTCACCGTC |
| | | | TCCTCA |
| 1056 | ATX-P-3365 | VH nt | CAGGTGCAGCTGCAGGAGTCGGGCCCAGGACTGGTGAAGCCTTC |
| | | | GGGGACCCTGTCCCTCACCTGCGCTGTCTCTGGTGACTCCATCAG |
| | | | CAGTAATAACTGGTGGAGTTGGGTCCGCCAGCCCCCAGGGAAGG |
| | | | GGCTGGAGTGGATTGGACAAATCTATCATAGTGGCCACACCAACT |
| | | | ACAACCCGTCCCTCAAGAGTCGAGTCACCATATCGGTTGACATGT |
| | | | CCAAGAACCAGTTCTCCCTGAAGCTGACCTCTGTGACCGCCGCGG |
| | | | ACACGGCCGTGTATTACTGTGCGAGGGGGGCTACAGAAATTCTG |
| | | | GGGTACTTCGATCTCTGGGGCCGTGGCACCCTGGTCACTGTCTCC |
| | | | TCA |
| 1057 | ATX-P-3367 | VH nt | CAGGTGCAGCTGCAGGAGTCGGGCCCAGGACTGGTGAAGCCTTC |
| | | | GGAGACCCTGTCCCTCACCTGCACTGTCTCTGGTGGCTCCGTCAG |
| | | | CAGTGGTAGTTACTACTGGAGCTGGATCCGGCAGCCCCCAGGGA |
| | | | AGGGACTGGAGTGGATTGGATATATCTATAAAAGTGGGTTCACC |
| | | | AATTACAACCCCTCCCTCAAGAGTCGAGTCACCATATCAGTAGAC |
| | | | ACGTCCAAGAACCAGTTCTCCCTGAAGCTGAGCTCTGTGACCGCT |
| | | | GCGGACACGGCCGTGTATTACTGTGCGATAGGGGGACGTGGATA |
| | | | CAGCTATGGTCCTTTTGACTACTGGGGCCAGGGAACCCTGGTCAC |
| | | | CGTCTCCTCA |
| 1058 | ATX-P-3369 | VH nt | CAGGTGCAGCTGGTGCAGTCTGGGGCTGAGGTGAAGAAGCCTGG |
| | | | GGCCTCAGTGAAGGTCTCCTGCAAGGCTTCTGGATACACCTTCAC |
| | | | CGGCTACTATTTACACTGGGTGCGACAGGCCCCTGGACAAGGACT |
| | | | TGAGTGGATGGGATGGATCAACCCTAACAGTGGTGACACAAACT |
| | | | ATGTACAGAACTTTCAGGGCAGGGTCACCATGACCAGGGACACG |
| | | | TCCATCAGCACAGCCTACATGGAGCTGAGCAGGCTGAGATCTGA |
| | | | CGACACGGCCGTGTATTACTGTGCGAGATCCTACGTGGATACCAG |
| | | | CTCGTCGGCTTTTGATATCTGGGGACAAGGGACAATGGTCACCGT |
| | | | CTCTTCA |
| 1059 | ATX-P-3370 | VH nt | CAGGTGCAGCTGCAGGAGTCGGGCCCAGGACTGGTGAAGCCTTC |
| | | | GGAGACCCTGTCCCTCACCTGCACTGTCTCTGGTGGCTCCATCAG |
| | | | TAGTTACTACTGGAGCTGGATCCGGCAGCCCCCCGGGAAGGGAC |
| | | | TGGAGTGGATTGGGTATGTCTATTACAGTGGGTACACCAAGTACA |
| | | | ACCCCTCCCTCAAGAGTCGAGTCACCATATCAGTAGACACGCCCA |
| | | | AGACCCAGTTCTCCCTGAAGATGAGCTCTGTGACCGCTGCGGACA |
| | | | CGGCCGTGTATTACTGTGCGAGCGTCGAATCAAAGTGGAACTACG |
| | | | GGCCTTTTGATATCTGGGGACAAGGGACAATGGTCACCGTCTCTT |
| | | | CA |
| 1060 | ATX-P-3371 | VH nt | CAGGTGCAGCTGCAGGAGTCGGGCCCAGGACTGGTGAAGCCTTC |
| | | | GGAGACCCTGTCCCTCACCTGCACTGTCTCTGGTGACTCCATCAG |
| | | | TAGTTACTACTGGAGCTGGATCCGGCAGCCCCCAGGGAAGGGAC |
| | | | TGGAGTGGATTGGATATATCTATTACAGTGGGTTCACCAAGTACA |
| | | | ACCCCTCCCTCAACAGTCGAGTCACCATATCAGTAGACACGTCCA |
| | | | AGAGCCAGTTCTCCCTGAAGCTGACCTCTGTGACCGCTGCGGACA |
| | | | CGGCCGTGTATTACTGTGCGATGAGAGAGCCGAGAGGCCGGTAT |
| | | | AACTGGAACTACGGGCCTTTTGATATCTGGGGACAAGGGACAAT |
| | | | GGTCACCGTCTCTTCA |
| 1061 | ATX-P-3373 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | TAATGATAACTGGTGGAGTTGGGTCCGCCAGCCCCCCGGGAAGG |
| | | | GACTGGAGTGGATTGGGAAAATCTATCATAGTGAGTTCACCAACT |
| | | | ACAACCCGTCCCTCAAGAGTCGAGTCACCATATCAGTAGACAAGT |
| | | | CCAAGAACCAGTTCTCCCTGGAGCTGAACTCTGTGACCGCCGCGG |
| | | | ACACGGCCGTGTATTACTGTGCGAGAGAGTATCATAATTTCATGG |
| | | | ACGTCTGGTGCAAAGGGACCACGGTCACCGTCTCCTCA |
| 1062 | ATX-P-3375 | VH nt | CAGGTGCAGCTGCAGGAGTCGGGCCCAGGACTGGTGAAGCCTTC |
| | | | GGAGACCCTGTCCCTCACCTGCACTGTCTCTGGTGACTCCGTCAG |
| | | | CAGTGGTAAATACTTCTGGAGCTGGATCCGGCAGTCCCCAGGGA |
| | | | AGGGACTGGAGTGGATTGGGTATATCTATTACAGTGGGAGCACC |
| | | | AACTACAACCCCTCCCTCAAGAATCGAGTCACCATATCGGTAGAC |
| | | | ACGTCCAAGAAGGAGTTGTCTCTGAAGCTGAGTTCTGTGACCGCT |
| | | | GCGGACACGGCCGTTTATTACTGTGCGAGAGGAGGGCATAACTG |
| | | | GAACTACGCTGCTTTTGATATCTGGGGACAAGGGACAATGGTCAC |
| | | | CGTCTCTTCA |
| 1063 | ATX-P-3378 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | TAATGATAACTGGTGGAGTTGGGTCCGCCAGTCCCCCGGGAAGG |
| | | | GGTTGGAGTGGATTGGGAAAATCTATCATAGTGAGTTCACCAACT |
| | | | ACAACCCGTCCCTCAAGAGTCGAGTCACCATATCAGTAGACAAGT |
| | | | CCAAGAACCAGTTCTCCCTGGAGCTGAGCTCTGTGGCCGCCGCGG |
| | | | ACACGGCCGTGTATTACTGTGCGAGAGAGTATGAGAACTTCATGG |
| | | | ACGTCTGGGGCAAGGGGACCACGGTCACCGTCTCCTCA |
| 1064 | ATX-P-3382 | VH nt | CAGGTGCAGCTGCAGGAGTCGGGCCCAGGACTGGTGAAGCCTTC |
| | | | GGGGACCCTGTCCCTCACCTGCGCTGTCTCTGGTGACTCCATCAG |
| | | | CAGTAATAGCTGGTGGAATTGGGTCCGCCAGCCCCCAGGGAAGG |
| | | | GGCTGGAGTGGATTGGGCAAATCTTTCATAGTGGACACACCAACT |
| | | | ACAACCCGTCCCTCAAGAGTCGAGTCACCATGTCTATAGACAAGT |
| | | | CCAAGAACCAGTTCTCCCTGAAACTGACCTCTGTGACCGCCGCGG |
| | | | ACACGGCCCTGTATTACTGTGCGAGAGGAGGGGAGTATGAAAAC |
| | | | TCGGCGAACTGGTTCGACCCCTGGGGCCAAGGAACCCTGGTCACC |
| | | | GTCTCTTCA |
| 1065 | ATX-P-3383 | VH nt | CAGGTGCAGCTGCAGGAGTCGGGCCCAGGACTGGTGAAGCCTTC |
| | | | GGGGACCCTGTCCCTCACCTGCGCTGTCTCTGGTGTCTCCATCAA |
| | | | CAAGGATAACTGGTGGACTTGGGTCCGCCAGCCCCCAGGGAAGG |
| | | | GGCTGGAGTGGATTGGGAAAATCTATCATAGTGGGAGCACCAAC |
| | | | TACAACCCGTCCCTCAAGAGTCGAGTCACCATATCAGTAGACAAC |
| | | | TCCAAGAACCAATTCTCCCTGAAGCTGAACTCTGTGACCGCCGCG |
| | | | GACACGGCCGTGTATTACTGTACGAGAGGGGGGGATATAGCTAT |
| | | | GGAGGGGGACTGGTTCGCCCCCTGGGGCCAGGGAACCCTGGTCA |
| | | | CCGTCTCCTCA |
| 1066 | ATX-P-3384 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | CAGTGATAACTGGTGGAATTGGGTCCGCCAGCCCCCAGGGAAGG |
| | | | GGCTGGAGTGGCTTGGAAAAATCTATCATAGTGGGAACACCGAC |
| | | | TACAACCCGTCCCTCAAGAGTCGAGTCACCATATCAGTAGACAAG |
| | | | TCCAAGAATCAGTTCTCCCTGAGGCTGAGTTCTGTGACCGCCGCG |
| | | | GACACGGCCGTGTATTACTGTGCGAGAGGTGGAACTGAAACTAC |
| | | | GGGGTACATGGACGTCTGGGGCAAAGGGACCACGGTCACCGTCT |
| | | | CCTCA |
| 1067 | ATX-P-3386 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | CAATGATAACTGGTGGAGTTGGGTCCGCCAGCCCCCAGGGAAGG |
| | | | GGCTGGAATGGATTGGGAAAATCTATCATAGTGGGTTCACCAACT |
| | | | ACAACCCGTCCCTCAAGAGTCGAGTCACCATATCAGTAGACAAGT |
| | | | CCAAGAACCAGTTCTCCCTGAAGCTGAACTCTGTGACCGCCGCGG |
| | | | ACACGGCCGTGTATTACTGTGCGAGAGGGGGGGATAACTTTATG |
| | | | GAGGGGGAGGGGTTAGGGACCTGGGGCCCGGGAACCCTGGTCAC |
| | | | CGTCTCCTCA |
| 1068 | ATX-P-3387 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | CAATGATAACTGGTGGAATTGGGTCCGCCAGTCCCCAGGGAAGG |
| | | | GGCTGGAGTGGATTGGAAAAATCTATCATAGTGGGTTCACCGACT |
| | | | ACAACCCGTCCCTCAAGAGTCGAGTCACCATATCAGTAGACAAGT |
| | | | CCAAGAACCAGTTCTCCCTGAAGCTGAGCTCTGTGACCGCCGCGG |
| | | | ACACGGCCGTGTATTATTGTGCGAGAGGAAACCATAACTATTTGT |
| | | | GGATTGGGTATTTGGGGACCTGGGGCCCGGGAACCCTGGTCACC |
| | | | GTCTCCTCA |
| 1069 | ATX-P-3389 | VH nt | CAGGTGCAGCTGCAGGAGTCGGGCCCAGGACTGGTGAAGCCTTC |
| | | | GGGGACCCTGTCCCTCATCTGCGCTGTCTCTGGTGGCTCCATCAG |
| | | | TAGTAGTAATTGGTGGAGTTGGGTCCGCCAGCCCCCAGGGAAGG |
| | | | GGCTGGAGTGGATTGGGAGAATCTATCATAGTGGGCACACCAAC |
| | | | TACAACCCGTCCCTCAAGAGTCGAGTCACCATATCAGTAGACAAG |
| | | | TCCAAGAACCAGTTCTCCCTGAAGCTGACCTCTGTGACCGCCGCG |
| | | | GACACGGCCGTGTATTACTGTGCGAGAGGGAGGGACTTAACTGG |
| | | | ATACTTTGACTACTGGGGCCAGGGAACCCTGGTCACCGTCTCCTC |
| | | | A |
| 1070 | ATX-P-3391 | VH nt | CAGGTGCAGCTGCAGGAGTCGGGCCCAGGACTGGTGAAACCTTC |
| | | | GGGGACCCTGTCCCTCACCTGCGCTGTCTCTGGTGGCTCCATCAG |
| | | | TAATGATAACTGGTGGAGTTGGGTCCGCCAGTCCCCCGGGAAGG |
| | | | GGCTGGAGTGGATTGGGAAAATCTATCATAGTGAGTTCACCAACT |
| | | | ACAACCCGTCCCTCAAGAGTCGAGTCACCATATCAGTAGACAAGT |
| | | | CCAAGAACCAGTTCTCCCTGGAGCTGAGCTCTGTGACCGCCGCGG |
| | | | ACACGGCCGTATATTACTGTGCGAGAGGGGTCGATTTTTGGAGTG |
| | | | GTTCCCACATGGACGTCTGGGGCAAAGGGACCACGGTCACCGTCT |
| | | | CCTCA |
| 1071 | ATX-P-3392 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | TAATGATAACTGGTGGAGTTGGGTCCGCCAGTCCCCCGGGAAGG |
| | | | GGCTGGAGTGGATTGGGAAAATCTATCATAGTGAGTTCACCAACT |
| | | | ACAACCCGTCCCTCAAGAGTCGAGTCACCATATCAGTAGACAAGT |
| | | | CTAAGAACCAGTTTTCCCTGGAACTGAGCTCTGTGACCGCCGCGG |
| | | | ACACGGCCGTGTATTTCTGTGCGCGAGAAGGACTGGAACTACCCC |
| | | | ACATGGACGTCTGGGGCAAAGGGACCACGGTCACCGTCTCCTCA |
| 1072 | ATX-P-3395 | VH nt | GAGGTGCAGCTGGTGGAGTCCGGGGGAGGCGTGGTCAAGCCTGG |
| | | | GGGGTCCCTGAGACTCTCCTGTGCAGCCTCTGGATTCACCTTCAG |
| | | | TAGCTACTGGATGCACTGGGTCCGCCAAGCTCCAGGGAAGGGGC |
| | | | TGGTGTGGGTCTCACGTATTAATAGTGATGGAAGTAGCACAAGCT |
| | | | ACGCGGACTCCGTGCAGGGCCGATTCACCATCTCCAGAGACATCG |
| | | | CCAAGAACACGCTGTATCTGCAAATGAACAGTCTGAGAGCCGAG |
| | | | GACACGGCTGTGTATTACTGTGCAAGAGGGCAGCACCTGGTTCAG |
| | | | GACTATTGGGGCCAGGGAACCCTGGTCACCGTCTCTTCA |
| 1073 | ATX-P-3396 | VH nt | CAGGTGCAGCTGGTGCAGTCTGGGGCTGAGGTGAAGAACCCTGG |
| | | | GGCCTCAGTGAAGGTCTCCTGCAAGGCTTCTGGATACACCTTCAC |
| | | | CGGCTACTATATGCACTGGGTGCGACAGGCCCCTGGACAAGGGC |
| | | | TTGAGTGGATGGGATGGATCAACCCTAACAGTGGTGGCACAAAC |
| | | | TATGCACAGAAGTTTCAGGGCAGGGTCACCATGACCAGGGACAC |
| | | | GTCCATCAGCACAGCCTACATGGAGCTGAGCAGGCTGAGATCTG |
| | | | ACGACACGGCCGTGTATTACTGTGCGAGAGAACGGGGCGACTAT |
| | | | GATGGCGATGCTTTTGATATCTGGGGACAAGGGACAATGGTCACC |
| | | | GTCTCTTCA |
| 1074 | ATX-P-3397 | VH nt | GAGGTGCAGCTGGTGGAGTCTGGGGGAGGCTTGGTCCAGCCTGG |
| | | | GGGGTCCCTGAGACTCTCCTGTGCAGCCTCTGGATTCACCTTTAG |
| | | | TAACTATTACATGAACTGGGTCCGCCAGGCTCCAGGGAAGGGGC |
| | | | TGGAGTGGGTGGCCAACATAAAGCAAGATGGAAGTGAGAAATAC |
| | | | TATGTGGACTCTGTGAAGGGCCGCTTCACCATCTCCAGAGACAAC |
| | | | GCCAAGAACTCACTGTATCTCCAAATGAACAGCCTGAGAGCCGA |
| | | | GGACACGGCTGTGTATTACTGTGCGAGAGATGGGTACTACAACTT |
| | | | CGATGCTTTTGATATCTGGGGACAAGGGACAATGGTCACCGTCTC |
| | | | TTCA |
| 1075 | ATX-P-3405 | VH nt | GAGGTGCAGCTGGTGGAGTCTGGGGGAGGCTTGGTACAGCCTGG |
| | | | GGGGTCCCTGAGACTCTCCTGTGCAGCCTCTGGATTCACCTTCAG |
| | | | TAGCTATAGCATGAACTGGGTCCGCCAGGCTCCAGGGAAGGGGC |
| | | | TGGAGTGGGTCTCATCCATTAGTAGTAGTAGTAGTTACATATACT |
| | | | ACGCAGACTCAGTGAAGGGCCGATTCACCATCTCCAGAGACAAC |
| | | | GCCAAGAACTCACTGTATCTGCAAATGAACAGCCTGAGAGCCGA |
| | | | GGACACGGCCGTGTATTACTGTGCGAGAGAAGGGTATGATAACT |
| | | | GGAACTCTGCTTGTGATATCTGGGGACAAGGGACAATGGTCACC |
| | | | GTCTCTTCA |
| 1076 | ATX-P-3406 | VH nt | CAGATCACCTTGAAGGAGTCTGGTCCTACGCTGGTGAAACCCACA |
| | | | CAGACCCTCACGCTGACCTGCACCTTCTCTGGGTTTTCACTCAGC |
| | | | ACTAGTGGAGTGGGTGTGGGCTGGATCCGTCAGCCCCCAGGAAA |
| | | | GGCCCTGGAGTGGCTTGCACTCATTTATTGGAATGGTGATGAGCG |
| | | | CTACAGCCCATCTCTGAAGAGCAGGCTCACCATCACCAAGGACA |
| | | | CCTCCAAAAACCAAGTGGTCCTTACAATGACCAACATGGACCCTG |
| | | | TGGACACAGCCACATATTACTGTGCACACAGTCCGAACTGGGGAT |
| | | | ATGAAGCTTTTGATATCTGGGGACAAGGGACAATGGTCACCGTCT |
| | | | CTTCA |
| 1077 | ATX-P-3407 | VH nt | GAGGTGCAGCTGGTGGAGTCTGGGGGAGGCCTGGTCCCGCCTGG |
| | | | GGGGTCCCTGAGACTCTCCTGTGCAGCCTCTGGATTCACCTTCAG |
| | | | TAGCTATTGCATGAACTGGGTCCGCCAGGCTCCAGGGAAGGGGC |
| | | | TGGAGTGGGTCTCATCCATTAATAGTGATAGTAGTTACATATACT |
| | | | ACGCGGACTCAGTGAAGGGCCGATTCACCATCTCCAGAGACAAC |
| | | | GCCAAGAACTCACTGTATCTGCAAATGAACAGCCTGAGAGCCGA |
| | | | GGACACGGCTGTGTATTACTGTGCGAGAGAGGGATACTACAACT |
| | | | GGAACGGAGGGAATGACTACTGGGGCCAGGGAACCCTGGTCACC |
| | | | GTCTCCTCA |
| 1078 | ATX-P-3408 | VH nt | GAGGTGCAGCTGGTGGAGTCCGGGGGAGGCTTAGTTCAGCCTGG |
| | | | GGGGTCCCTGAGACTCTCCTGTGCAGCCTCTGGATTCACCTTCAG |
| | | | TAGCTATTGGATGCACTGGGTCCGCCAAGCTCCAGGGAAGGGGC |
| | | | TGGTGTGGGTCTCACATATTAATAGTGATGGGAGTAGCACAAACT |
| | | | ACGCGGACTCCGTGAAGGGCCGATTCACCATCTCCAGAGACAAC |
| | | | GCCAAGAACACGCTGTATCTGCAGATGAACAGTCTGAGATCCGA |
| | | | GGACACGGCTGTATATTACTGTGCAAGAGAGGGTTACTGGAACG |
| | | | GTGATACTTTTGATATCTGGGGACAAGGGACAATGGTCACCGTCT |
| | | | CTTCA |
| 1079 | ATX-P-3410 | VH nt | GAGGTGCAGCTGGTGGAGTCTGGGGGAGGCTTGGTACAGCCTGG |
| | | | GGGGTCCCTGAGACTCTCCTGTGCAGCCTCTGGATTCACCTTCAG |
| | | | TGCCTACGACATGCACTGGGTCCGCCAAACTCCAGGAAAAGGTCT |
| | | | GGAGTGGGTCTCAACTATTGGTACTGCTGGTGACACATACTATCC |
| | | | AGACTCCGTGAAGGGCCGATTCACCATCTCCAGAGATTTTGCCAG |
| | | | GAGGTCCTTGTATCTTCACATGAACAATCTGAGAGCCGGGGACAC |
| | | | GGCTGTATATTACTGTGCAAGAGGGGGCAGCAGCAGCTGGTACG |
| | | | ACTGGTACTTCGATCTCTGGGGCCGTGGCACCCTGGTCACTGTCT |
| | | | CCTCA |
| 1080 | ATX-P-3412 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | CAGTAGTACCTGGTGGAGTTGGGTCCGCCAGCCCCCAGGGAAGG |
| | | | GGCTGGAGTGGATTGGGGAAATCTATCATAGTGGGAGCACCAAC |
| | | | TATAACCCGTCCCTCAAGAGTCGAGTCACCATATCACTAGACAAG |
| | | | TCCAAGAACCAGTTTTCCCTGAAGCTGAGCTCTGTGACCGCCGCG |
| | | | GACACGGCCGTGTATTACTGTGCGATTAGTTTATACAACTGGAAC |
| | | | GATGCTGCTTTTGATATCTGGGGACAAGGGACAATGGTCACCGTC |
| | | | TCTTCA |
| 1081 | ATX-P-3413 | VH nt | CAGGTGCAGCTGGTGCAGTCTGGGTCTGAGGTGAAGAAGCCTGG |
| | | | GGCCTCAGTGAAGGTCTCCTGCAAGACTTCTGGATACACGTTCAC |
| | | | CGGCTACTATATGCACTGGGTGCGACAGGCCCCTGGACAAGGGC |
| | | | TTGAGTGGATGGGATGGATCAACCCTAACAGTGGTGGCACAACC |
| | | | TATGCACAGAAGTTTCAGGGCAGGGTCACCATGACCAGGGACAC |
| | | | GTCCATCAGCACAGCCTACATGGAGCTGAACAGGCTGAGATCTG |
| | | | ACGACACGGCCGTGTATTACTGTGCGAGAGAGGCAGGTGGTTCA |
| | | | GGTGAATACTTCCAGCACTGGGGCCAGGGCACCCCGGTCACCGTC |
| | | | TCTTCA |
| 1082 | ATX-P-3414 | VH nt | CAGATCACCTTGAAGGAGTCTGGTCCTACGCTGGTGAAACCCACA |
| | | | CAGACCCTCACGCTGACCTGCACCTTCTCTGGGTTCTCACTCAGC |
| | | | ACTAGTGGAGTGGGTGTGGGCTGGATCCGTCAGCCCCCAGGAAA |
| | | | GGCCCTGGAGTGGCTTGCACTCATTTATTGGAATGATGTTAAGTT |
| | | | CTACAGTCCATCTCTGCAGAGCAGGCTCACCATCACCAAGGACAC |
| | | | CTCCAAAAACCAGGTGGTCCTTACACTGACCAACATGGACCCTGT |
| | | | GGACACAGCCACATATTACTGTGCACACGGGGATAACTGGAACT |
| | | | ACCGTGCTTTTGATATCTGGGGACAAGGGACAATGGTCACCGTCT |
| | | | CTTCA |
| 1083 | ATX-P-3419 | VH nt | CAGGTTCAGCTGGTGCAGTCTGGAGCTGAGGTGAAGAAGCCTGG |
| | | | GGCCTCAGTGAAGGTCTCCTGCAAGGCTTCTGGTTACACCTTTAC |
| | | | CAGCTATGGTATCAGCTGGGTGCGACAGGCCCCTGGACAAGGGC |
| | | | TTGAGTGGATGGGATGGATCAGCGCTTATAATGGTAACACACACT |
| | | | ATGCACAGAACCTCCAGGGCAGAGTCACCATGACCACAGACACA |
| | | | TCCACGAGCACAGCCTACATGGAGCTGAGGAGCCTGAGATCTGA |
| | | | CGACACGGCCGTGTATTACTGTGCGAGGAGTATAGCAGCCCTTGA |
| | | | CTTCTGGGGCCAGGGAACCCCGGTCACCGTCTCCTCA |
| 1084 | ATX-P-3421 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | CAGTAATAACTGGTGGAGTTGGGTCCGCCAGCCCCCAGGGAAGG |
| | | | GACTGGAGTGGATTGGACAAATCTATCATAGTGAAAACACCAAC |
| | | | TACAACCCGTCCCTCAAGAGTCGAGTCACCATATCAGTAGACAAG |
| | | | TCCAAGAACCAGTTCTCCCTGAAGCTGAGCTCTGTGACCGCCGCG |
| | | | GACACGGCCATCTATTACTGTGCGAGAGTGGATGACTGGAACTAC |
| | | | GGGGGTCACTTTGACTACTGGGGCCAGGGAACCCTGGTCACCGTC |
| | | | TCCTCA |
| 1085 | ATX-P-3428 | VH nt | CAGGTTCAGCTGGTGCAGTCTGGAGCTGAGGTGAAGAAGCCTGG |
| | | | GGCCTCAGTTAAGGTCTCCTGCAAGGCTTCTGGTTACACCTTTAC |
| | | | CGGCTATGGTATCAGCTGGGTGCGACAGGCCCCTGGACAAGGGC |
| | | | TTGAGTGGATGGGATGGATCAGCGCTTACAATGGTAACACACACT |
| | | | ATGCACAGAACCTCCAGGGCAGAGTCACCATGACCACCGACACA |
| | | | TCCACGAACACAGCCTACATGGAGCTGAGGAGCCTTATATCTGAC |
| | | | GACACGGCCTTTTATTATTGTGCGAGAGCGGGACTGGGGATCGAC |
| | | | TACTGGGGCCAGGGAACCCTGGTCACTGTCTCCTCA |
| 1086 | ATX-P-3430 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | CAGTAGTGACTGGTGGAGTTGGGTCCGCCAGTCCCCAGGGAAGG |
| | | | GGCTGGAGTGGATTGGGGAAATCTATCATAGTGGGAGCACCAAC |
| | | | TACAACCCGTCCCTCAAGAGTCGAGTCTCCATATCAGTAGACAAG |
| | | | TCCAAGAACCAGTTCTCCCTGAAGCTGAGCTCTGTGACCGCCGCG |
| | | | GACACGGCCGTGTATTACTGTGCGAGAGGGGGGCACATAGGCTA |
| | | | CTACTACATGGACGTCTGGGGCAAAGGGACCACGGTCACCGTCTC |
| | | | CTCA |
| 1087 | ATX-P-3431 | VH nt | GAGGTGCAGCTGGTGCAGTCTGGAGCAGAGGTGAAAAAGCCCGG |
| | | | GGAGTCTCTGAAGATCTCCTGTAAGGGTTCTGGATACAGCTTTAC |
| | | | CAACTACTGGATCGGCTGGGTGCGCCAGATGCCCGGGAAAGGCC |
| | | | TGGAGTGGATGGGGATCATCTATCCTGGTGACTCTGATACCATAT |
| | | | ACAGCCCGTCCTTCCAAGGCCAGGTCACCTTCTCAGCCGACAAGT |
| | | | CCATCAACACCGCCTACCTGCAGTGGAGCAGCCTGAAGGCCTCG |
| | | | GACACCGCCATGTATTACTGTGCGAGAGGACGTGGATATAGTGG |
| | | | CTACGAAGGTGCTTTTGATATCTGGGGACAAGGGACAATGGTCAC |
| | | | CGTCTCTTCA |
| 1088 | ATX-P-3435 | VH nt | CAGGTTCAGCTGGTGCAGTCTGGAGCTGAGGTGAAGAAGCCTGG |
| | | | GGCCTCAGTGAAGGTCTCCTGCAAGGCTTCTGGTTACACCTTTAC |
| | | | CAACTATGGTATCAGCTGGGTGCGACAGGCCCCTGGACAAGGGC |
| | | | TTGAGTGGATGGGATGGATCAGCGCTTACAATGGTAACACACACT |
| | | | ATGCACAGAAACTCCAGGGCAGAGTCACCATGACCACAGACACA |
| | | | TCCACGAGCACAGCCTACATGGAGCTGAGGAGCCTGAGATCAGA |
| | | | CGACACGGCCGTGTATTACTGTGCGAGAGCGGCAGCAGCCGTTG |
| | | | ACTACTGGGGCCAGGGAACCCTGGTCACCGTCTCTTCA |
| 1089 | ATX-P-3436 | VH nt | CAGGTGCAGCTGCAGGAGTCGGGTCCAGGACTGGTGAAGCCTTC |
| | | | GGAGACCCTGTCCCTCAGTTGCACTGTCTCTGGTGGCTCCGTCAG |
| | | | CAGTGGTAGTTACTACTGGAGCTGGATCCGGCAGCCCCCAGGGA |
| | | | AGGGACTGGAGTGGATTGGGTATATCTATTACAGTGGGAGCACC |
| | | | AAGTACAACCCCTCCCTCAAGAGTCGAGTCACCATATCAGTAGAC |
| | | | ACGTCCAAGAACCAGGTCTCCCTGAAGCTGAGTTCTGTGACCGCT |
| | | | GCGGACACGGCCGTGTATTACTGTGCGAGAGATAAGGGGCACTG |
| | | | GAACTACGGTGCTTTTGATATCTGGGGACAAGGGACAATGGTCAC |
| | | | CGTCTCTTCA |
| 1090 | ATX-P-3437 | VH ni | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | CAGTAATAACTGGTGGAGTTGGGTCCGCCAGCCCCCAGGGAAGG |
| | | | GGCTGGAGTGGATTGGGGAAATCTATCATAGTGGAAATATTAACT |
| | | | ACAACCCGTCCCTCAAGAGTCGAGTCACCATATCAGTGGACAAGT |
| | | | CCAAGAACCAGITCTCCCTGAAGCTGAGCTCTGTGACCGCCGCGG |
| | | | ACACGGCCGTGTATTACTGTGCGAGAGGGGGGGAATACGGTGAC |
| | | | CCCGTGGGGTACTTCGATCTCTGGGGCCGTGGCACCCTGGTCACT |
| | | | GTCTCCTCA |
| 1091 | ATX-P-3440 | VH nt | CAGGTTCAGCTGGTGCAGTCTGGAGCTGAGGTGAAGAAGCCTGG |
| | | | GGCCTCAGTGAAGGTCTCCTGCAAGGCTTCTGGTTACACCTTTAC |
| | | | CACCTATGGTATCAGCTGGGTGCGACAGGCCCCTGGACAAGGGC |
| | | | TTGAGTGGATGGGATGGATCAGCGCTTACAGTGGTAACACAAAC |
| | | | TATGCACAGAAGCTCCAGGGCAGAGTCACCATGACCACAGACAC |
| | | | ATCCACGAGCACAGCCTACATGGAGCTGAGGAGCCTGAGATCTG |
| | | | ACGACACGGCCGTGTATTACTGTGCGAGAGAGAGAGTCTCCAAC |
| | | | TGGGTCTTTGACTTCTGGGGCCAGGGAACCCTGGTCACTGTCTCC |
| | | | TCA |
| 1092 | ATX-P-3442 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGTTCCATCAG |
| | | | CAGTAGTGACTGGTGGAGCTGGGTCCGCCAGCCCCCAGGGAAGG |
| | | | GGCTGGAGTGGATTGGGGAAATCTATCATAGTAGGAACACCAAC |
| | | | TACAACCCGTCCCTCAAGAGTCGAGTCACCATATCAGTAGACAAG |
| | | | TCCAAGAACCAGTTCTCCCTGAAGCTGAGTTCTGTGACCGCCGCG |
| | | | GACACGGCCGTGTATTACTGTGCGAGTGGAGGTGTGGTGGCCGA |
| | | | CTACTACATGGACGTCTGGGGCAAAGGGACCACGGTCACCGTCTC |
| | | | CTCA |
| 1093 | ATX-P-3444 | VH nt | GAGGTGCAGCTGGTGGAGTCTGGGGGAGGCCTGGTCAAGCCTGG |
| | | | GGGGTCCCTGAGACTCTCCTGTGCAGCCTCTGGATTCACCTTCAG |
| | | | TAGCTATGGCATGAACTGGGTCCGCCAGGCTCCAGGGAAGGGGC |
| | | | TGGAGTGGGTCTCATCCATTAGTGGTTATGGTAGTTACATATACT |
| | | | ACGCAGCCTCAGTGAAGGGCCGATTCACCATCTCCAGAGACAGC |
| | | | GCCAAGAACTCACTGTATCTGCAAATGAACAGCCTGAGCGCCGA |
| | | | GGACACAGCTGTGTATTACTGTGCGAGAGAATTAGGTGGGATCTA |
| | | | CTACGGGGTAGGGTACTTCGATCTCTGGGGCCGTGGCACCCTGGT |
| | | | CACTGTCTCCTCA |
| 1094 | ATX-P-3447 | VH nt | CAGGTGCAGCTGCAGGAGTCGGGCCCAGGACTGGTGAAGCCTTC |
| | | | GGAGACCCTGTCCCTCACCTGCGCTGTCTCTGGTGGCTCCGTCAG |
| | | | CAGTGGTAATTACTATTGGAGCTGGATCCGGCAGCCCCCAGGGA |
| | | | AGGGACTGGAGTGGATTGGGTATATCTATTCCAGTGGGAGCACC |
| | | | AACTACAACCCCTCCCTCAAGAGTCGAGTCACCATATCAATAGAC |
| | | | ACGTCCAAGAACCAGTTCTCCCTGAAGCTGAGCTCTGTGACCGCT |
| | | | GCGGACACGGCCGTGTATTACTGTGCGAGAAGTCCTCGTGGATAC |
| | | | AGCTATGGTCCCTTTGCCTACTGGGGCCAGGGGACCCTGGTCACC |
| | | | GTCTCTTCA |
| 1095 | ATX-P-3448 | VH nt | CAGGTGCAGCTGCAGGAGTCGGGCCCAGGACTGGTGAAGCCTTC |
| | | | ACAGACCCTGTCCCTCACCTGCACTGTCTCTGGTGGCTCCATCAG |
| | | | CAGTGGTGGTTACAACTGGAACTGGATCCGCCAGCACCCAGGGA |
| | | | AGGGCCTGGAATGGATTGGGTACATCAATTACAGTGGGAATACC |
| | | | TATTACAACCCGTCCCTCAAGAGTCGAGTTACCATATCAGTAGAC |
| | | | ACGTCTAAGAACCAGTTCTCCCTGAAGCTGAGTTCTGTGACTGCC |
| | | | GCGGACACGGCCGTTTATTACTGTGCGAGAGGGCCCCTGGATACA |
| | | | GCTATGGCCCCCTTTGACTACTGGGGCCAGGGAACCCTGGTCACC |
| | | | GTCTCCTCA |
| 1096 | ATX-P-3449 | VH nt | CAGGTGCAGCTGCAGGAGTCGGGCCCAGGACTGGTGAAGCCTTC |
| | | | GGAGACCCTGTCCCTCACCTGTACTGTCTCTGGTGACTCCATCAG |
| | | | TAGTTACTACTGGAGTTGGATCCGGCAGCCCCCAGGGAAGGGAC |
| | | | TGGAGTGGATTGGATATATCTATTATAGTGGGTTCACCAAGTACA |
| | | | ACCCCTCCCTCAAGAGTCGAGTCACCATTTCAGTAGACACGTCCA |
| | | | AGAACCAGTTCTCCCTGAGGCTGAGCTCTGTGACCGCTGCGGACA |
| | | | CGGCCGTGTATTACTGTGCGAGCGTCGATTCAAAGTGGAACTACG |
| | | | GGTCTTTTCATATCTGGGGACAAGGGACAATGGTCACCGTCTCTT |
| | | | CA |
| 1097 | ATX-P-3450 | VH nt | CAGGTGCAGCTGCAGGAGTCGGGCCCAGGGCTGGTGAAGCCTTC |
| | | | GGAGACCCTGTCCCTCACCTGCTCTGTCTCTGGTGACTCCGTCAG |
| | | | CAATAATCGTTATTATTGGAGCTGGATTCGGCAGCCCCCAGGGAA |
| | | | GGGACTGGAGTGGATTGGGTATATCTATTACAGTGGGAGCACCA |
| | | | ACTACAACCCCTCCCTCAAGAGTCGAGTCACCATATCAGTAGACA |
| | | | CGTCCAAGAACCAGTTGTCCCTGAACCTGAGTTCTGTGACCGCTG |
| | | | CGGACACGGCCGTGTATTACTGTGCGAGAGATCGCCCATCCCGGT |
| | | | ATAAGTGGAATTACGATGCTTTTGCTATCTGGGGACAAGGGACAA |
| | | | TGGTCACCGTCTCTTCA |
| 1098 | ATX-P-3451 | VH nt | CAGGTGCAGCTACAGCAGTGGGGCGCAGGACTGTTGAAGCCTTC |
| | | | GGAGACCCTGTCCCTCACCTGCGCTGTCTATGGTGGGTCCTTCAG |
| | | | TGATTACTACTGGACCTGGATCCGCCAGCCCCCAGGGAAGGGGCT |
| | | | GGAGTGGATTGGTGAAGTCAATCATATTGGAAGCACCAACTACA |
| | | | ACCCGTCCCTCAAGAGTCGAGTCACCATATCAGTAGACACGTCCA |
| | | | AGAACCAGTTCTCCCTGAACCTGAGCTCTGTGACCGCCGCGGACA |
| | | | CGGCTGTGTATTACTGTGCGAAGTATAGCACTTCCTGGTACGGCT |
| | | | GGTTCGACCCCTGGGGCCAGGGAACCCTGGTCACTGTCTCCTCA |
| 1099 | ATX-P-3453 | VH nt | CAGGTGCAGCTGCAGGAGTCGGGCCCAGGGCTGGTGAAGCCTTC |
| | | | GGGGACCCTGTCCCTCACCTGCGCTGTCTCTGGTGACTCCATCAG |
| | | | CAGTAATAACTGGTGGAGTTGGTTCCGCCAGCCCCCAGGGAAGG |
| | | | GACTGGAGTGGATTGGGAGAATCTATCATAGTGGGACCACCAAC |
| | | | TACAACCCGTCCCTCGAGAGTCGAGTCACCATATCAGTAGACAAC |
| | | | TCCAGGAACCAGTTCTCCCTGAAACTGAGCTCTGTGACCGCCGCG |
| | | | GACACGGCCGTGTATTACTGTGCGAGAGGCGAGGAGTGGGAGCT |
| | | | GCGGGGAGGTGTTTTTGATATCTGGGGACAAGGGACAATGGTCA |
| | | | CCGTCTCTTCA |
| 1100 | ATX-P-3454 | VH nt | CAGGTGCAGCTGGTGCAATCTGGGCCTGAGTTGAAGAAGCCTGG |
| | | | GGCCTCAGTAAAGGTTTCCTGCAAGGCTTCTGGATACACCTTCAC |
| | | | TAACTATGCTGTGAATTGGGTGCGACAGGCCCCTGGACAAGGGCT |
| | | | TGAGTGGATGGGATGGATCAACACCAACACTGGGCACCCAACGT |
| | | | ATGCCCAGGGCTTCACAGGACGGTTTGTCTTCTCCTTGGACACCT |
| | | | CTGTCAGTACGGCATATCTGCAGATCAGCAGCCTAAAGGCTGAG |
| | | | GACACTGCCGTTTATTACTGTGCGAGAGACAGCAGCAGCTGGGG |
| | | | CTACTGGGGCCAGGGAACCCTGGTCACTGTCTCCTCA |
| 1101 | ATX-P-3455 | VH nt | CAGGTGCAGCTGCAGGAGTCGGGCCCAGGACTGGTGACGCCTTC |
| | | | GGGGACCCTGTCCCTCACCTGCGCTGTCTCTGGTGACTCCATCAG |
| | | | TAGTGATAACTGGTGGAGTTGGGTCCGCCAGCCCCCAGGGAAGG |
| | | | GACTGGAGTGGATTGGGGAAATCTATCACAGTGAAAACACCAAC |
| | | | TACAACCCGTCCCTCAAGAGTCGAGTCACCATATCAGTAGACAAG |
| | | | TCCAAGAACCAGTTCTCCCTGAGGCTGAACTCTGTGACCGCCGCG |
| | | | GACACGGCCATGTATTACTGTGCGAGAGGGAGGGAGCTAACTGG |
| | | | CCTCTTTGACTACTGGGGCCAGGGAACCCTGGTCACCGTCTCCTC |
| | | | A |
| 1102 | ATX-P-3456 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | CAGTAATAACTGGTGGAGTTGGGTCCGCCAGCCCCCAGGAAAGG |
| | | | GACTGGAATGGATTGGGGACATCTATCATAGTGGAAACACCAAC |
| | | | TACAACCCGTCCCTCAAGAGTCGAGTCACCCTATCAGTAGACATG |
| | | | TCCAAGAACCAGTTCTCCCTGAAACTGAACTCTGTGACCGCCGCG |
| | | | GACACGGCCGTGTATTACTGTGCGAGAGGAGGTATAGCAGTGAC |
| | | | TGCCTACTTTGACTACTGGGGCCAGGGAACCCTGGTCACCGTCTC |
| | | | CTCA |
| 1103 | ATX-P-3458 | VH nt | CAGGTGCAGCTGGTGCAGTCTGGGGCTGAGGTGAAGAAGCCTGG |
| | | | GGCCTCAGTGAAGGTCTCCTGCAAGGCTTCTGGATACACCTTCAC |
| | | | CGGCTACTATTTGCACTGGGTGCGACAGGCCCCTGGACAAGGGCT |
| | | | TGAGTGGATGGGATGGATCAACCCTCACAGTGGTGGCACAATCT |
| | | | ATGCACAGAAGTTTCAGGACAGGGTCACCATGACCAGGGACACG |
| | | | TCCATCAGCACAGCCTACATGGAGCTGAGCAGGCTGAGATCTGA |
| | | | CGACACGGCCGTGTATTACTGTGCGAACTGGAACTCCGGGGACTC |
| | | | CTACTACTACTACATGGACGTCTGGGGCAAAGGGACCACGGTCA |
| | | | CCGTCTCCTCA |
| 1104 | ATX-P-3459 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGTTCCATCAG |
| | | | CAGTGATAACTGGTGGAATTGGGTCCGCCAGCCCCCAGGGAAGG |
| | | | GGCTGGAGTGGATTGGAAAAATCTATCATAGTGGGAACACCAAC |
| | | | TACAACCCGTCCCTCAAGAGTCGAGTCACCATATCAGTAGACGAG |
| | | | TCCAAGAACCAGTTCTCCCTGAGGCTGTACTCTGTGACCGCCGCG |
| | | | GACACGGCCGTGTATTACTGTGCGAGAGGGGTCGATTTTTGGAGT |
| | | | GGTTCCCACATGGACGTCTGGGGCAAAGGGACCACGGTCACCGT |
| | | | CTCCTCA |
| 1105 | ATX-P-3462 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATTAG |
| | | | CAGTAATAATTGGTGGAGTTGGGTCCGCCAGGCCCCAGGGAAGG |
| | | | GGCTGGAGTGGATTGGGAAGATCTATCAAAGTGGGAGCACCATG |
| | | | TACAACCCGTCCCTCAAGAGTCGAGTCACCATGTCGGTAGACAAG |
| | | | TCCAAGAACCAGTTCTCCCTGATTCTGAGTTCTGTGACCGCCGCG |
| | | | GACACGGCCGTGTATTACTGTGCGAGAGATAAACCCCCTTTACTG |
| | | | GGAGCCTTTGACTACTGGGGCCAGGGAACCCTGGTCACTGTCTCC |
| | | | TCA |
| 1106 | ATX-P-3463 | VH nt | CAGGTGCAGCTGGTGGAGTCTGGGGGAGGCGTGGTCCAGCCTGG |
| | | | GAGGTCCCTGAGACTCTCCTGTGCAGCCTCTGGATTCACCTTCAG |
| | | | TAGCTATGCCATGCACTGGGTCCGCCAGGCTCCAGGCAAGGGGCT |
| | | | GGAGTGGGTGGCAGTTATATCATATGATGGAAGTAATAAATACT |
| | | | ATGCGGACTCCGTGAAGGGCCGATTCACCATCTCCAGAGACAATT |
| | | | CCAAGAACACGCTGTCTCTGCAAATGAACAGCCTGAGAGCTGAG |
| | | | GACACGGCTGTGTATTACTGTGCGAAAAGGGGGGGATACAGCTA |
| | | | TGGTCCTTTTGAACACTGGGGCCAGGGAACCCTGGTCACCGTCTC |
| | | | CTCA |
| 1107 | ATX-P-3466 | VH nt | CAGGTGCAGCTGGTGCAATCTGGGTCTGAATTGAAGAAGCCTGG |
| | | | GGCCTCAGTGAAGGTTTCCTGCAAGGCTTCTGGATACACCTTCAC |
| | | | TAGCTATACTATGAATTGGGTGCGACAGGCCCCTGGACAAGGGCT |
| | | | TGAGTGGGTGGGATGGATCAACACCGCCACTGGGACCCCAACGT |
| | | | ATGCCCAGGGCTTCACAGGACGGTATGTCTTCTCCTTGGACACCT |
| | | | CTGTCAGCACGGCTTATCTGCAGATCAGCGGCCTAAAGGCTGAGG |
| | | | ACACTGCCGTGTATTTCTGTGCGAGGTATAGTGGGAGCCACTCCT |
| | | | TTGACTACTGGGGCCAGGGAACCCTGGTCACCGTCTCTTCA |
| 1108 | ATX-P-3467 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | TAGTGATTACTGGTGGAGTTGGGTCCGCCAGTCCCCAGGGAAGG |
| | | | GGCTGGAGTGGATTGGGAAAATCTATCATAGTGGGAACACCAAC |
| | | | CACAACCCGTCCCTCAAGAGTCGAGTCACCATATCAATAGACAA |
| | | | GTCCAGGAACCAGTTCTCCCTGAATCTGAACTCTGTGACCGCCGC |
| | | | GGACACGGCCGTGTATTACTGTGCGAGAGAAGGACTGGAACTTC |
| | | | CCCACATGGACGTCTGGGGCAAAGGGACCACGGTCACCGTCTCCT |
| | | | CA |
| 1109 | ATX-P-3468 | VH nt | CAGGTGCAGCTGGTGGAGTCTGGGGGAGGCTTGGTCCAGCCTGG |
| | | | GAGGTCCCTGAGACTCTCCTGTGCAGCCTCTGGATTCACCTTCAG |
| | | | TAGTTATGCTATGCACTGGGTCCGCCAGGCTCCAGGCAAGGGGCT |
| | | | GGAGTGGGTGACAGTTATATCATATGATGGAAGTAATAAATACT |
| | | | ATGCAGACTCCGTGAAGGGCCGATTCACCATCTCCAGAGACAATT |
| | | | CCAAGAACACGCTGTATCTGCAAATGAACAGCCTGAGAGCTGAG |
| | | | GACACGGCTGTGTATTACTGTGCGAAAAGGGGTAACTGGAACTA |
| | | | CGGACCTTTTCACTACTGGGGCCAGGGAACCCTGGTCACTGTCTC |
| | | | CTCA |
| 1110 | ATX-P-3469 | VH nt | CAGGTGCAGCTGGTGGAGTCTGGGGGAGGCGTGGTCCAGCCTGG |
| | | | GAGGTCCCTGAGACTCTCCTGTGCAGCCTCTGGATTCACCTTCAG |
| | | | TGCCTATGCCATCCACTGGGTCCGCCAGGCTCCAGGCAAGGGGCT |
| | | | GGAGTGGGTGGCAGTTATATCATATGATGGAAGTAATAAAAAGT |
| | | | ATGCAGACTCCGTGAAGGGCCGATTCACCATCTCCAGAGACAATT |
| | | | CCAAGAACACGCTGGATCTGGAAATGAACAGCCTGAGAGCTGAG |
| | | | GACACGGCTGTGTACTACTGTGCGAAAGGGCTACCTTTTGACTAC |
| | | | TGGGGCCAGGGAACCCTGGTCACCGTCTCCTCA |
| 1111 | ATX-P-3470 | VH nt | CAGGTGCAGCTGGTGCAATCTGGGTCTGACTTGAAGAAGCCTGG |
| | | | GGCCTCAGTGAAGGTTTCCTGCAAGGCTTCTGGATACACCTTCAC |
| | | | TAGTTATACTATGAATTGGGTGCGACAGGCCCCTGGACAAGGGCT |
| | | | TGAGTGGATGGGATGGATCAACACCAACACTGGGACCCCAACGT |
| | | | ATGCCCAGGGCTTCACAGGACGGTTTGTCTTCTCCTTGGACACCT |
| | | | CTGTCAGCACGGCATATCTGCAGATCAGCAGCCTAAAGGCTGAG |
| | | | GACACTGCCGTGTATTACTGTACGAGGTATACTGGGAACTACTCC |
| | | | TTTGACTACTGGGGCCAGGGAACCCTGGTCACCGTCTCCTCA |
| 1112 | ATX-P-3471 | VH nt | GAGGTGCAGCTGGTGGAGTCTGGGGGAGTCTTGGTACAGCCTGG |
| | | | GGGGTCCCTGAGACTCTCCTGTGCAGCCTCTGGATTCACCTTTAG |
| | | | GAGCTATGCCATGAGCTGGGTCCGCCAGGCTCCAGGGAAGGGGC |
| | | | TGGAGTGGGTCTCAGCTATTAGTGGTAGTGGTGATAGCACATACT |
| | | | TCGCAGACTCCGTGAAGGGCCGGTTCACCATCTCCAGAGACAATT |
| | | | CCAAGAACACGCTGTCTCTGCAAATGAACAGCCTGAGAGCCGAG |
| | | | GACACGGCCGTATATTACTGTGCGAGCATCCTAGGATATTGTAGT |
| | | | GGTACCAGCTGCTATGGCCTCTTTGACAACTGGGGCCAGGGAACC |
| | | | CTGGTCACCGTCTCCTCA |
| 1113 | ATX-P-3472 | VH nt | CAGGTGCAGCTGCAGGAGTCGGGCCCAGGACTGGTGAAGCCTTC |
| | | | GGGGACCCTGTCCCTCACCTGCGCTGTCTCTGGTGGCTCCATCAG |
| | | | TAATGATAACTGGTGGAGTTGGGTCCGCCAGTCCCCCGGGAAGG |
| | | | GGCTGGAGTGGATTGGGAAAATCTATCATAGTGAGTTCACCAACT |
| | | | ACAACCCGTCCCTCAGGAGTCGAGTCACCATATCAGTAGACAAGT |
| | | | CCAAGAACCAGTTCTCCCTGGAGCTGACCTCTGTGACCGCCGCGG |
| | | | ACACGGCCGTGTATTACTGTGCGAGAGAGTATCAGAACTACATG |
| | | | GACGTCTGGGGCAAAGGGACCACGGTCACCGTCTCCTCA |
| 1114 | ATX-P-3361 | VH nt | CAGGTGCAGCTGCAGGAGTCGGGCCCAGGACTGGTGAAGCCTTC |
| | | | GGGGACCCTGTCCCTCACCTGCGTTGTCTCTGGTGACTCCATCAG |
| | | | CAGTAATAACTGGTGGAGTTGGGTCCGCCAGCCCCCAGGGAAGG |
| | | | GTCTGGAGTGGATTGGACAAATCTATCATAGTGGAATCACCAACT |
| | | | ACAACCCGTCCCTCAAGAGTCGAGTCACCATATCAGTAGACAAGT |
| | | | CCAAGAACCAGTTCTCCCTGGAGCTGACCTCTGTGACCGCCGCGG |
| | | | ACGCGGCCGTGTATTACTGTGCGAGGGCCCAGTGGGAACTACGA |
| | | | GCGGGCTGGTTCGACCCCTGGGGCCAGGGAACCCTGGTCACCGTC |
| | | | TCCTCA |
| 1115 | ATX-P-3366 | VH nt | GAGGTGCAGCTGGTGGAGTCTGGGGGAGGCTTGGTAAAGCCTGG |
| | | | GGGGACCCTTACACTCTCCTGTGCAGCCTCTGGATTCACTTTCAGT |
| | | | AACGCCTGGATGAGCTGGGTCCGCCAGGCTCCAGGGAAGGGGCT |
| | | | GGAGTGGGTTGGCCGTATTAAAAGCAAAACTGATGGCGGGACAA |
| | | | CAGACTACGCTGCACCCGTGAAAGGCAGATTCACCATCTCAAGA |
| | | | GATGATTCAAAAAACACGCTGTATCTGCAAATGAACAGCCTGAA |
| | | | AACCGAGGACACAGCCGTGTATTACTGTACCCCCCGGTACAACTC |
| | | | GGACTTTGACTACTGGGGCCAAGGAACCCTGGTCACCGTCTCTTC |
| | | | A |
| 1116 | ATX-P-3376 | VH nt | CAGGTGCAGCTGCAGGAGTCGGGCCCAGGACTGGTGAAGCCTTC |
| | | | GGAGACCCTGTCCCTCACCTGCACTGTCTCTGGTGGCTCCATCAG |
| | | | TAGTTACTACTGGAGCTGGATCCGGCAGCCCCCAGGGAAGGGAC |
| | | | TGGAGTGGATTGGGTATATCTATTACAGTGGGTTCACCAAGTACA |
| | | | ACCCCTCCCTCAAGAGTCGAGTCACCATATCAGTAGACACGTCCA |
| | | | AGAACCAGTTCTCCCTGAAGCTGAGCTCTGTGACCGCTGCGGACA |
| | | | CGGCCGTGTATTACTGTGCGACCCACCCTCGTGGATACAACTATG |
| | | | GTCCCTTTGACTACTGGGGCCAGGGAACCCTGGTCACTGTCTCCT |
| | | | CA |
| 1117 | ATX-P-3377 | VH nt | CAGGTGCAGCTGCAGGAGTCGGGCCCAGGACTGGTGAAGCCTTC |
| | | | GGAGACCCTGTCCCTCACCTGCACTGTCTCTGGTGGCTCCGTCAA |
| | | | CAGTGGTCGTTACTACTGGAGCTGGATCCGGCAGCCCCCAGGGA |
| | | | AGGGACTGGAGTGGATTGGGTATATCTATTCCAGTGGGTACACCA |
| | | | ACTACAACCCCTCCCTCAAGAGTCGAGTCACCATGTTACTAGACA |
| | | | CGTCCAAGAACCAGTTCTCCCTGAAGCTGAACTCCGTGACCGCTG |
| | | | CGGACACGGCCGTGTATTACTGTGCGAGGTCACGTGGATACCGCT |
| | | | ATGGTCCCTTTGACTACTGGGGCCAGGGAACCCTGGTCACCGTCT |
| | | | CCTCA |
| 1118 | ATX-P-3418 | VH nt | CAGGTGCAGCTGGTGCAATCTGGGTCTGAGTTGAAGAAGCCTGG |
| | | | GGCCTCAGTGAAGGTTTCCTGCGAGGCTTCTGGATACACCTTCAC |
| | | | TAGCTATGCTATGAATTGGGTGCGACAGGCCCCTGGACAAGGGCT |
| | | | TGAGTGGATGGGATGGATCAACACCAACACTGGGACCCCAACTT |
| | | | ATGCCCAGGGCTTCACAGGACGGTTTGTCTTCTCCTTGGACACCT |
| | | | CTGTCAGCACGGCATATCTGCAGATCAGCAGCCTAAAGGCTGAG |
| | | | GACACTGCCGTGTATTACTGTGCGAGTCATAGCACCTCGTTTGAC |
| | | | TACTGGGGCCAGGGGACCCTGGTCACCGTCTCCTCA |
| 1119 | ATX-P-3420 | VH nt | CAGGTGCAGCTACAGCAGTGGGGCGCAGGACTGGTGAAGCCTTC |
| | | | GGAGACCCTGTCCCTCACCTGCGCTGTCCATGGTGGGTCCTTCAG |
| | | | TGATTACTACTTGACCTGGATTCGCCAGCCCCCAGGGAAGGGGCT |
| | | | GGAGTGGATTGGGGAAATCAATCATAGTGGAAGCACCAACTACA |
| | | | ACCCCTCCCTCAAGAGTCGAGTCACCTTATCAATAGACACGTCCA |
| | | | AGAACCAGTTCTCCCTGAAGCTGAGCTCTGTGACCGCCGCGGACA |
| | | | CGGCTGTGTATTACTGTGCGAGAGGTAGTGAAGACTACTACTATT |
| | | | ACATGGACGTCTGGGGCAAAGGGACCACGGTCACCGTCTCCTCA |
| 1120 | ATX-P-3426 | VH nt | GAGGTGCAGCTGGTGCAGTCTGGAGCAGAGGTGAAAAAGCCCGG |
| | | | GGAGTCTCTGAAGATCTCCTGTAAGGGTTCTGGATACATCTTTAC |
| | | | CAGCTACTGGATCGGCTGGGTGCGCCAGATGCCCGGGAAAGGCC |
| | | | TGGAGTGGATGGGGATCATCTATCCTGGTGACTCTGATACCAGAT |
| | | | ACAGCCCGTCTTTCCAAGGCCAGGTCACCATCTCAGCCGACAAGT |
| | | | CCATCATCACCGCCTACCTGCAGTGGAGCAGCCTGAAGGCCTCGG |
| | | | ACACCGCCATGTATTACTGTGCGAGACACGACTACTTTGATATTG |
| | | | GAGGTTATTTTGACTACTGGGGCCAGGGAACCCTGGTCACCGTCT |
| | | | CCTCA |
| 1121 | ATX-P-3432 | VH nt | CAGGTGCAGCTGGTGCAGTCTGGGGCTGAGGTGAAGAAGCCTGG |
| | | | GGCCTCAGTGAAGGTCTCCTGCAAGGCTGATGGATACACCTTCAC |
| | | | CGACTACTATATGCACTGGGTGCGACAGGCCCCTGGACAAGGGC |
| | | | ATGAGTGGATGGGATGGATCAACCCTAACAGTGGTGTCACATACT |
| | | | ATGCACAGAAGTTTCAGGGCAGGGTCACCATGACCAGGGACACG |
| | | | TCCATCAGCACAGCCTACATGGAGCTGAACAGACTTAGATCTGAC |
| | | | GACACGGCCGTGTATTACTGTGCGAGAGATCGGACCTTCTACTTT |
| | | | GACTACTGGGGCCAGGGAACCCTGGTCACTGTCTCCTCA |
| 1122 | ATX-P-3433 | VH nt | CAGGTGCAGCTGGTGCAGTCTGGGGCTGAGGTGAAGAAGCCTGG |
| | | | GGCCTCAGTGAAGGTCTCCTGCAAGGCTTCTGGATACACCTTCAC |
| | | | CGACTACTATATGCACTGGGTGCGACAGGCCCCTGGACAAGGGC |
| | | | TTGAGTGGATGGGATGGATCAACCCTAACAATGGTGGCACAGAC |
| | | | TATGCACAGAAGTTTCAGGGCAGGGTCACCATGACCAGGGACAC |
| | | | GTCCATCAGCACAGCCTACATGGAGTTACACAGGCTAAGATCTGA |
| | | | CGACACGGCCGTGTATTTCTGTGCGAGAGGGGTCTATGATGTAGT |
| | | | AGTTCCAACTGCTATCGTCAACTGGTTCGACCCCTGGGGCCAGGG |
| | | | AACCCTGGTCACCGTCTCCTCA |
| 1123 | ATX-P-3438 | VH nt | CAGGTGCAGCTGCAGGAGTCGGGCCCAGGACTGGTGAAGCCTTC |
| | | | GGGGACCCTGTCCCTCACCTGCGCTGTCTCTGGTGACTCCATCAG |
| | | | CAATGATTACTGGTGGAATTGGGTCCGCCAGCCCCCAGGGAAGG |
| | | | GTCTGGAGTGGATTGGAGAAATCTATCATAGTGGGCACACCAACT |
| | | | ACAACCCGTCCCTCAAGAGTCGAGTCACCATATCAGTAGACAAGT |
| | | | CCAAGAACCAGTTGTCCCTGAAGCTGACCTCTGTGAACGCCGCGG |
| | | | ACACGGCCGTGTATTACTGTGCGAGAGGTGGAACTGAAATTACG |
| | | | GGGTACATGGACGTCTGGGGCAAAGGGACCACGGTCACCGTCTC |
| | | | CTCA |
| 1124 | ATX-P-3441 | VH nt | CAGGTGCAGCTGCAGGAGTCGGGCCCAGGACTGGTGAAGCCTTC |
| | | | GGGGACCCTGTCCCTCACCTGCGCTGTCTCTGGTGCCTCCATCAG |
| | | | AAGTGGTCACTGGTGGAGTTGGGTCCGCCAGCCCCCAGGGAAGG |
| | | | GACTGGAGTGGATTGGGGAAATCTATCATAGTGGGAACACCAAC |
| | | | TACAACCCGTCCCTCAAGAGTCGAGTCACCCTATCAGTAGACAAG |
| | | | TCCAAGAACCAGTTCTCCCTGAAGCTGAGCTCTGTGACCGCCGCG |
| | | | GACACGGCCGTGTATTACTGTGCGAGAGGTCACTACGGTGACTAC |
| | | | GTCGTCTACTTTGACTACTGGGGCCAGGGAACCCTGGTCACTGTC |
| | | | TCCTCA |
| 1125 | ATX-P-3446 | VH nt | CAGGTGCAGCTGCAGGAGTCGGGCCCAGGACTGGTGAAGCCTTC |
| | | | GGAGACCCTGTCCCTCACCTGCACTGTCTCTGGTGGCTCCATCAG |
| | | | TAGTTACTATTGGAGCTGGATCCGGCAGCCCCCAGGGAAGGGAC |
| | | | TGGAATGGATTGGTTATGTCTATTACAGTGGGTACACCAAGTACA |
| | | | ATCCCTCCCTCAAGAGTCGAGTCACCATATCAATAGACGCGTCCA |
| | | | AGAACCAGTTCTCCCTGAAGCTGACCTCTGTGACCGCTGCGGACA |
| | | | CGGCCGTGTATTACTGTGCGACCGTCGAATCAAAGTGGAACTACG |
| | | | GGCCTTTTGCTATCTGGGGACAAGGGACAATGGTCACCGTCTCTT |
| | | | CA |
| 1126 | ATX-P-3452 | VH nt | CAGGTGCAGCTGGTGCAGTCTGGGGCTGAGGTGAAGAAGCCTGG |
| | | | GGCCTCAGTGAAGGTCTCCTGCAAGGCTTCTGGATACACCTTCAC |
| | | | CGGCTACTATATGAACTGGGTGCGACAGGCCCCTGGACAAGGGC |
| | | | TTGAGTGGATGGGATGGATCAACCCTAACAGTGGTGCCACAAAC |
| | | | TATGCACAGAAGTTTCAGGGCAGGGTCACCATGACCAGGGACAC |
| | | | GTCCATCAGCACAGACTACATGGAACTGAGCAGGCTGAGATCTG |
| | | | ACGACACGGCCGTTTATTACTGTGCGAGAGATGGGGCTAAGTGG |
| | | | AATCACGAGGGTTACTACTTTGACTACTGGGGCCAGGGAACCCTG |
| | | | GTCACCGTCTCCTCA |
| 1127 | ATX-P-3360 | VH aa | QVQLQESGPGLVKPSGTLSLTCAVSGGSISINNWWSWVRQPPGKGL |
| | | | EWLGKIYHSGNTDYNPSLKSRVTISVDKSKNQFSLRLSSVTAADTAV |
| | | | YYCASGSDYDFWSGYLDYWGQGTLVTVSS |
| 1128 | ATX-P-3362 | VH aa | EVQLVESGGGLVQPGRSLRLSCAASGFTFDDYAMHWVRQAPGKGL |
| | | | EWVSGISWNSGSIAYADSVKGRFTISRDNAKNSLYLQMNSLRVEDT |
| | | | AFFYCARVAAAAGIPFFDYWGQGTLVTVSS |
| 1129 | ATX-P-3363 | VH aa | QVQLQESGPGLVKPSGTLTLTCAVSGGSINRDNWWSWVRQPPGKGL |
| | | | EWIGQIYQSGSTNYNPSLKSRVTISVDKSKNQFSLKLSSVTAADTAV |
| | | | YYCARVYDWNYGGHFDYWGQGTLVTVSS |
| 1130 | ATX-P-3365 | VH aa | QVQLQESGPGLVKPSGTLSLTCAVSGDSISSNNWWSWVRQPPGKGL |
| | | | EWIGQIYHSGHTNYNPSLKSRVTISVDMSKNQFSLKLTSVTAADTAV |
| | | | YYCARGATEILGYFDLWGRGTLVTVSS |
| 1131 | ATX-P-3367 | VH aa | QVQLQESGPGLVKPSETLSLTCTVSGGSVSSGSYYWSWIRQPPGKGL |
| | | | EWIGYIYKSGFTNYNPSLKSRVTISVDTSKNQFSLKLSSVTAADTAVY |
| | | | YCAIGGRGYSYGPFDYWGQGTLVTVSS |
| 1132 | ATX-P-3369 | VH aa | QVQLVQSGAEVKKPGASVKVSCKASGYTFTGYYLHWVRQAPGQGL |
| | | | EWMGWINPNSGDTNYVQNFQGRVTMTRDTSISTAYMELSRLRSDD |
| | | | TAVYYCARSYVDTSSSAFDIWGQGTMVTVSS |
| 1133 | ATX-P-3370 | VH aa | QVQLQESGPGLVKPSETLSLTCTVSGGSISSYYWSWIRQPPGKGLEWI |
| | | | GYVYYSGYTKYNPSLKSRVTISVDTPKTQFSLKMSSVTAADTAVYY |
| | | | CASVESKWNYGPFDIWGQGTMVTVSS |
| 1134 | ATX-P-3371 | VH aa | QVQLQESGPGLVKPSETLSLTCTVSGDSISSYYWSWIRQPPGKGLEWI |
| | | | GYIYYSGFTKYNPSLNSRVTISVDTSKSQFSLKLTSVTAADTAVYYC |
| | | | AMREPRGRYNWNYGPFDIWGQGTMVTVSS |
| 1135 | ATX-P-3373 | VH aa | QVQLQESGPGLVKPSGTLSLTCAVSGGSISNDNWWSWVRQPPGKGL |
| | | | EWIGKIYHSEFTNYNPSLKSRVTISVDKSKNQFSLELNSVTAADTAV |
| | | | YYCAREYHNFMDVWCKGTTVTVSS |
| 1136 | ATX-P-3375 | VH aa | QVQLQESGPGLVKPSETLSLTCTVSGDSVSSGKYFWSWIRQSPGKGL |
| | | | EWIGYIYYSGSTNYNPSLKNRVTISVDTSKKELSLKLSSVTAADTAV |
| | | | YYCARGGHNWNYAAFDIWGQGTMVTVSS |
| 1137 | ATX-P-3378 | VH aa | QVQLQESGPGLVKPSGTLSLTCAVSGGSISNDNWWSWVRQSPGKGL |
| | | | EWIGKIYHSEFTNYNPSLKSRVTISVDKSKNQFSLELSSVAAADTAVY |
| | | | YCAREYENFMDVWGKGTTVTVSS |
| 1138 | ATX-P-3382 | VH aa | QVQLQESGPGLVKPSGTLSLTCAVSGDSISSNSWWNWVRQPPGKGL |
| | | | EWIGQIFHSGHTNYNPSLKSRVTMSIDKSKNQFSLKLTSVTAADTAL |
| | | | YYCARGGEYENSANWEDPWGQGTLVTVSS |
| 1139 | ATX-P-3383 | VH aa | QVQLQESGPGLVKPSGTLSLTCAVSGVSINKDNWWTWVRQPPGKG |
| | | | LEWIGKIYHSGSTNYNPSLKSRVTISVDNSKNQFSLKLNSVTAADTA |
| | | | VYYCTRGGDIAMEGDWFAPWGQGTLVTVSS |
| 1140 | ATX-P-3384 | VH aa | QVQLQESGPGLVKPSGTLSLTCAVSGGSISSDNWWNWVRQPPGKGL |
| | | | EWLGKIYHSGNTDYNPSLKSRVTISVDKSKNQFSLRLSSVTAADTAV |
| | | | YYCARGGTETTGYMDVWGKGTTVTVSS |
| 1141 | ATX-P-3386 | VH aa | QVQLQESGPGLVKPSGTLSLTCAVSGGSISNDNWWSWVRQPPGKGL |
| | | | EWIGKIYHSGFTNYNPSLKSRVTISVDKSKNQFSLKLNSVTAADTAV |
| | | | YYCARGGDNFMEGEGLGTWGPGTLVTVSS |
| 1142 | ATX-P-3387 | VH aa | QVQLQESGPGLVKPSGTLSLTCAVSGGSISNDNWWNWVRQSPGKGL |
| | | | EWIGKIYHSGFTDYNPSLKSRVTISVDKSKNQFSLKLSSVTAADTAV |
| | | | YYCARGNHNYLWIGYLGTWGPGTLVTVSS |
| 1143 | ATX-P-3389 | VH aa | QVQLQESGPGLVKPSGTLSLICAVSGGSISSSNWWSWVRQPPGKGLE |
| | | | WIGRIYHSGHTNYNPSLKSRVTISVDKSKNQFSLKLTSVTAADTAVY |
| | | | YCARGRDLTGYFDYWGQGTLVTVSS |
| 1144 | ATX-P-3391 | VH aa | QVQLQESGPGLVKPSGTLSLTCAVSGGSISNDNWWSWVRQSPGKGL |
| | | | EWIGKIYHSEFTNYNPSLKSRVTISVDKSKNQFSLELSSVTAADTAVY |
| | | | YCARGVDFWSGSHMDVWGKGTTVTVSS |
| 1145 | ATX-P-3392 | VH aa | QVQLQESGPGLVKPSGTLSLTCAVSGGSISNDNWWSWVRQSPGKGL |
| | | | EWIGKIYHSEFTNYNPSLKSRVTISVDKSKNQFSLELSSVTAADTAVY |
| | | | FCAREGLELPHMDVWGKGTTVTVSS |
| 1146 | ATX-P-3395 | VH aa | EVQLVESGGGVVKPGGSLRLSCAASGFTFSSYWMHWVRQAPGKGL |
| | | | VWVSRINSDGSSTSYADSVQGRFTISRDIAKNTLYLQMNSLRAEDTA |
| | | | VYYCARGQHLVQDYWGQGTLVTVSS |
| 1147 | ATX-P-3396 | VH aa | QVQLVQSGAEVKNPGASVKVSCKASGYTFTGYYMHWVRQAPGQG |
| | | | LEWMGWINPNSGGTNYAQKFQGRVTMTRDTSISTAYMELSRLRSD |
| | | | DTAVYYCARERGDYDGDAFDIWGQGTMVTVSS |
| 1148 | ATX-P-3397 | VH aa | EVQLVESGGGLVQPGGSLRLSCAASGFTFSNYYMNWVRQAPGKGL |
| | | | EWVANIKQDGSEKYYVDSVKGRFTISRDNAKNSLYLQMNSLRAEDT |
| | | | AVYYCARDGYYNFDAFDIWGQGTMVTVSS |
| 1149 | ATX-P-3405 | VH aa | EVQLVESGGGLVQPGGSLRLSCAASGFTFSSYSMNWVRQAPGKGLE |
| | | | WVSSISSSSSYIYYADSVKGRFTISRDNAKNSLYLQMNSLRAEDTAV |
| | | | YYCAREGYDNWNSACDIWGQGTMVTVSS |
| 1150 | ATX-P-3406 | VH aa | QITLKESGPTLVKPTQTLTLTCTFSGFSLSTSGVGVGWIRQPPGKALE |
| | | | WLALIYWNGDERYSPSLKSRLTITKDTSKNQVVLTMINMDPVDTAT |
| | | | YYCAHSPNWGYEAFDIWGQGTMVTVSS |
| 1151 | ATX-P-3407 | VH aa | EVQLVESGGGLVPPGGSLRLSCAASGFTFSSYCMNWVRQAPGKGLE |
| | | | WVSSINSDSSYIYYADSVKGRFTISRDNAKNSLYLQMNSLRAEDTAV |
| | | | YYCAREGYYNWNGGNDYWGQGTLVTVSS |
| 1152 | ATX-P-3408 | VH aa | EVQLVESGGGLVQPGGSLRLSCAASGFTFSSYWMHWVRQAPGKGL |
| | | | VWVSHINSDGSSTNYADSVKGRFTISRDNAKNTLYLQMNSLRSEDT |
| | | | AVYYCAREGYWNGDTFDIWGQGTMVTVSS |
| 1153 | ATX-P-3410 | VH aa | EVQLVESGGGLVQPGGSLRLSCAASGFTFSAYDMHWVRQTPGKGLE |
| | | | WVSTIGTAGDTYYPDSVKGRFTISRDFARRSLYLHMNNLRAGDTAV |
| | | | YYCARGGSSSWYDWYFDLWGRGTLVTVSS |
| 1154 | ATX-P-3412 | VH aa | QVQLQESGPGLVKPSGTLSLTCAVSGGSISSSTWWSWVRQPPGKGLE |
| | | | WIGEIYHSGSTNYNPSLKSRVTISLDKSKNQFSLKLSSVTAADTAVYY |
| | | | CAISLYNWNDAAFDIWGQGTMVTVSS |
| 1155 | ATX-P-3413 | VH aa | QVQLVQSGSEVKKPGASVKVSCKTSGYTFTGYYMHWVRQAPGQGL |
| | | | EWMGWINPNSGGTTYAQKFQGRVTMTRDTSISTAYMELNRLRSDD |
| | | | TAVYYCAREAGGSGEYFQHWGQGTPVTVSS |
| 1156 | ATX-P-3414 | VH aa | QITLKESGPTLVKPTQTLTLTCTFSGFSLSTSGVGVGWIRQPPGKALE |
| | | | WLALIYWNDVKFYSPSLQSRLTITKDTSKNQVVLTLTNMDPVDTAT |
| | | | YYCAHGDNWNYRAFDIWGQGTMVTVSS |
| 1157 | ATX-P-3419 | VH aa | QVQLVQSGAEVKKPGASVKVSCKASGYTFTSYGISWVRQAPGQGLE |
| | | | WMGWISAYNGNTHYAQNLQGRVTMTTDTSTSTAYMELRSLRSDDT |
| | | | AVYYCARSIAALDFWGQGTPVTVSS |
| 1158 | ATX-P-3421 | VH aa | QVQLQESGPGLVKPSGTLSLTCAVSGGSISSNNWWSWVRQPPGKGL |
| | | | EWIGQIYHSENTNYNPSLKSRVTISVDKSKNQFSLKLSSVTAADTAIY |
| | | | YCARVDDWNYGGHFDYWGQGTLVTVSS |
| 1159 | ATX-P-3428 | VH aa | QVQLVQSGAEVKKPGASVKVSCKASGYTFTGYGISWVRQAPGQGL |
| | | | EWMGWISAYNGNTHYAQNLQGRVIMTTDTSTNTAYMELRSLISDD |
| | | | TAFYYCARAGLGIDYWGQGTLVTVSS |
| 1160 | ATX-P-3430 | VH aa | QVQLQESGPGLVKPSGTLSLTCAVSGGSISSSDWWSWVRQSPGKGL |
| | | | EWIGEIYHSGSTNYNPSLKSRVSISVDKSKNQFSLKLSSVTAADTAVY |
| | | | YCARGGHIGYYYMDVWGKGTTVTVSS |
| 1161 | ATX-P-3431 | VH aa | EVQLVQSGAEVKKPGESLKISCKGSGYSFTNYWIGWVRQMPGKGLE |
| | | | WMGIIYPGDSDTIYSPSFQGQVTFSADKSINTAYLQWSSLKASDTAM |
| | | | YYCARGRGYSGYEGAFDIWGQGTMVTVSS |
| 1162 | ATX-P-3435 | VH aa | QVQLVQSGAEVKKPGASVKVSCKASGYTFTNYGISWVRQAPGQGL |
| | | | EWMGWISAYNGNTHYAQKLQGRVTMTTDTSTSTAYMELRSLRSDD |
| | | | TAVYYCARAAAAVDYWGQGTLVTVSS |
| 1163 | ATX-P-3436 | VH aa | QVQLQESGPGLVKPSETLSLSCTVSGGSVSSGSYYWSWIRQPPGKGL |
| | | | EWIGYIYYSGSTKYNPSLKSRVTISVDTSKNQVSLKLSSVTAADTAV |
| | | | YYCARDKGHWNYGAFDIWGQGTMVTVSS |
| 1164 | ATX-P-3437 | VH aa | QVQLQESGPGLVKPSGTLSLTCAVSGGSISSNNWWSWVRQPPGKGL |
| | | | EWIGEIYHSGNINYNPSLKSRVTISVDKSKNQFSLKLSSVTAADTAVY |
| | | | YCARGGEYGDPVGYFDLWGRGTLVTVSS |
| 1165 | ATX-P-3440 | VH aa | QVQLVQSGAEVKKPGASVKVSCKASGYTFTTYGISWVRQAPGQGLE |
| | | | WMGWISAYSGNTNYAQKLQGRVTMTTDTSTSTAYMELRSLRSDDT |
| | | | AVYYCARERVSNWVFDFWGQGTLVTVSS |
| 1166 | ATX-P-3442 | VH aa | QVQLQESGPGLVKPSGTLSLTCAVSGGSISSSDWWSWVRQPPGKGL |
| | | | EWIGEIYHSRNTNYNPSLKSRVTISVDKSKNQFSLKLSSVTAADTAV |
| | | | YYCASGGVVADYYMDVWGKGTTVTVSS |
| 1167 | ATX-P-3444 | VH aa | EVQLVESGGGLVKPGGSLRLSCAASGFTFSSYGMNWVRQAPGKGLE |
| | | | WVSSISGYGSYIYYAASVKGRFTISRDSAKNSLYLQMNSLSAEDTAV |
| | | | YYCARELGGIYYGVGYFDLWGRGTLVTVSS |
| 1168 | ATX-P-3447 | VH aa | QVQLQESGPGLVKPSETLSLTCAVSGGSVSSGNYYWSWIRQPPGKG |
| | | | LEWIGYIYSSGSTNYNPSLKSRVTISIDTSKNQFSLKLSSVTAADTAV |
| | | | YYCARSPRGYSYGPFAYWGQGTLVTVSS |
| 1169 | ATX-P-3448 | VH aa | QVQLQESGPGLVKPSQTLSLTCTVSGGSISSGGYNWNWIRQHPGKGL |
| | | | EWIGYINYSGNTYYNPSLKSRVTISVDTSKNQFSLKLSSVTAADTAV |
| | | | YYCARGPLDTAMAPFDYWGQGTLVTVSS |
| 1170 | ATX-P-3449 | VH aa | QVQLQESGPGLVKPSETLSLTCTVSGDSISSYYWSWIRQPPGKGLEWI |
| | | | GYIYYSGFTKYNPSLKSRVTISVDTSKNQFSLRLSSVTAADTAVYYC |
| | | | ASVDSKWNYGSFHIWGQGTMVTVSS |
| 1171 | ATX-P-3450 | VH aa | QVQLQESGPGLVKPSETLSLTCSVSGDSVSNNRYYWSWIRQPPGKGL |
| | | | EWIGYIYYSGSTNYNPSLKSRVTISVDTSKNQLSLNLSSVTAADTAV |
| | | | YYCARDRPSRYKWNYDAFAIWGQGTMVTVSS |
| 117 | ATX-P-3451 | VH aa | QVQLQQWGAGLLKPSETLSLTCAVYGGSFSDYYWTWIRQPPGKGLE |
| | | | WIGEVNHIGSTNYNPSLKSRVTISVDTSKNQFSLNLSSVTAADTAVY |
| | | | YCAKYSTSWYGWFDPWGQGTLVTVSS |
| 1173 | ATX-P-3453 | VH aa | QVQLQESGPGLVKPSGTLSLTCAVSGDSISSNNWWSWFRQPPGKGL |
| | | | EWIGRIYHSGTTNYNPSLESRVTISVDNSRNQFSLKLSSVTAADTAVY |
| | | | YCARGEEWELRGGVFDIWGQGTMVTVSS |
| 1174 | ATX-P-3454 | VH aa | QVQLVQSGPELKKPGASVKVSCKASGYTFTNYAVNWVRQAPGQGL |
| | | | EWMGWINTNTGHPTYAQGFTGRFVFSLDTSVSTAYLQISSLKAEDT |
| | | | AVYYCARDSSSWGYWGQGTLVTVSS |
| 1175 | ATX-P-3455 | VH aa | QVQLQESGPGLVTPSGTLSLTCAVSGDSISSDNWWSWVRQPPGKGL |
| | | | EWIGEIYHSENTNYNPSLKSRVTISVDKSKNQFSLRLNSVTAADTAM |
| | | | YYCARGRELTGLFDYWGQGTLVTVSS |
| 1176 | ATX-P-3456 | VH aa | QVQLQESGPGLVKPSGTLSLTCAVSGGSISSNNWWSWVRQPPGKGL |
| | | | EWIGDIYHSGNTNYNPSLKSRVTLSVDMSKNQFSLKLNSVTAADTA |
| | | | VYYCARGGIAVTAYFDYWGQGTLVTVSS |
| 1177 | ATX-P-3458 | VH aa | QVQLVQSGAEVKKPGASVKVSCKASGYTFTGYYLHWVRQAPGQGL |
| | | | EWMGWINPHSGGTIYAQKFQDRVTMTRDTSISTAYMELSRLRSDDT |
| | | | AVYYCANWNSGDSYYYYMDVWGKGTTVTVSS |
| 1178 | ATX-P-3459 | VH aa | QVQLQESGPGLVKPSGTLSLTCAVSGGSISSDNWWNWVRQPPGKGL |
| | | | EWIGKIYHSGNTNYNPSLKSRVTISVDESKNQFSLRLYSVTAADTAV |
| | | | YYCARGVDFWSGSHMDVWGKGTTVTVSS |
| 1179 | ATX-P-3462 | VH aa | QVQLQESGPGLVKPSGTLSLTCAVSGGSISSNNWWSWVRQAPGKGL |
| | | | EWIGKIYQSGSTMYNPSLKSRVTMSVDKSKNQFSLILSSVTAADTAV |
| | | | YYCARDKPPLLGAFDYWGQGTLVTVSS |
| 1180 | ATX-P-3463 | VH aa | QVQLVESGGGVVQPGRSLRLSCAASGFTFSSYAMHWVRQAPGKGL |
| | | | EWVAVISYDGSNKYYADSVKGRFTISRDNSKNTLSLQMNSLRAEDT |
| | | | AVYYCAKRGGYSYGPFEHWGQGTLVTVSS |
| 1181 | ATX-P-3466 | VH aa | QVQLVQSGSELKKPGASVKVSCKASGYTFTSYTMNWVRQAPGQGL |
| | | | EWVGWINTATGTPTYAQGFTGRYVFSLDTSVSTAYLQISGLKAEDT |
| | | | AVYFCARYSGSHSFDYWGQGTLVTVSS |
| 1182 | ATX-P-3467 | VH aa | QVQLQESGPGLVKPSGTLSLTCAVSGGSISSDYWWSWVRQSPGKGL |
| | | | EWIGKIYHSGNINHNPSLKSRVTISIDKSRNQFSLNLNSVTAADTAVY |
| | | | YCAREGLELPHMDVWGKGTTVTVSS |
| 1183 | ATX-P-3468 | VH aa | QVQLVESGGGLVQPGRSLRLSCAASGFTFSSYAMHWVRQAPGKGLE |
| | | | WVTVISYDGSNKYYADSVKGRFTISRDNSKNTLYLQMNSLRAEDTA |
| | | | VYYCAKRGNWNYGPFHYWGQGTLVTVSS |
| 1184 | ATX-P-3469 | VH aa | QVQLVESGGGVVQPGRSLRLSCAASGFTFSAY AIHWVRQAPGKGLE |
| | | | WVAVISYDGSNKKYADSVKGRFTISRDNSKNTLDLEMNSLRAEDTA |
| | | | VYYCAKGLPFDYWGQGTLVTVSS |
| 1185 | ATX-P-3470 | VH aa | QVQLVQSGSDLKKPGASVKVSCKASGYTFTSYTMNWVRQAPGQGL |
| | | | EWMGWINTNTGTPTYAQGFTGRFVFSLDTSVSTAYLQISSLKAEDTA |
| | | | VYYCTRYTGNYSFDYWGQGTLVTVSS |
| 1186 | ATX-P-3471 | VH aa | EVQLVESGGVLVQPGGSLRLSCAASGFTFRSYAMSWVRQAPGKGLE |
| | | | WVSAISGSGDSTYFADSVKGRFTISRDNSKNTLSLQMNSLRAEDTAV |
| | | | YYCASILGYCSGTSCYGLFDNWGQGTLVTVSS |
| 1187 | ATX-P-3472 | VH aa | QVQLQESGPGLVKPSGTLSLTCAVSGGSISNDNWWSWVRQSPGKGL |
| | | | EWIGKIYHSEFTNYNPSLRSRVTISVDKSKNQFSLELTSVTAADTAVY |
| | | | YCAREYQNYMDVWGKGTTVTVSS |
| 1188 | ATX-P-3461 | VH aa | QVQLQESGPGLVKPSGTLSLTCVVSGDSISSNNWWSWVRQPPGKGL |
| | | | EWIGQIYHSGITNYNPSLKSRVTISVDKSKNQFSLELTSVTAADAAVY |
| | | | YCARAQWELRAGWEDPWGQGTLVTVSS |
| 1189 | ATX-P-3466 | VH aa | EVQLVESGGGLVKPGGTLTLSCAASGFTFSNAWMSWVRQAPGKGL |
| | | | EWVGRIKSKTDGGTTDYAAPVKGRFTISRDDSKNTLYLQMNSLKTE |
| | | | DTAVYYCTPRYNSDFDYWGQGTLVTVSS |
| 1190 | ATX-P-3476 | VH aa | QVQLQESGPGLVKPSETLSLTCTVSGGSISSYYWSWIRQPPGKGLEWI |
| | | | GYIYYSGFTKYNPSLKSRVTISVDTSKNQFSLKLSSVTAADTAVYYC |
| | | | ATHPRGYNYGPFDYWGQGTLVTVSS |
| 1191 | ATX-P-3477 | VH aa | QVQLQESGPGLVKPSETLSLTCTVSGGSVNSGRYYWSWIRQPPGKG |
| | | | LEWIGYIYSSGYTNYNPSLKSRVTMLLDTSKNQFSLKLNSVTAADTA |
| | | | VYYCARSRGYRYGPFDYWGQGTLVTVSS |
| 1192 | ATX-P-3418 | VH aa | QVQLVQSGSELKKPGASVKVSCEASGYTFTSYAMNWVRQAPGQGL |
| | | | EWMGWINTNTGTPTYAQGFTGRFVFSLDTSVSTAYLQISSLKAEDTA |
| | | | VYYCASHSTSFDYWGQGTLVTVSS |
| 1193 | ATX-P-3420 | VH aa | QVQLQQWGAGLVKPSETLSLTCAVHGGSFSDYYLTWIRQPPGKGLE |
| | | | WIGEINHSGSTNYNPSLKSRVTLSIDTSKNQFSLKLSSVTAADTAVYY |
| | | | CARGSEDYYYYMDVWGKGTTVTVSS |
| 1194 | ATX-P-3426 | VH aa | EVQLVQSGAEVKKPGESLKISCKGSGYIFTSYWIGWVRQMPGKGLE |
| | | | WMGIIYPGDSDTRYSPSFQGQVTISADKSIITAYLQWSSLKASDTAM |
| | | | YYCARHDYFDIGGYFDYWGQGTLVTVSS |
| 1195 | ATX-P-3432 | VH aa | QVQLVQSGAEVKKPGASVKVSCKADGYTFTDYYMHWVRQAPGQG |
| | | | HEWMGWINPNSGVTYYAQKFQGRVTMTRDTSISTAYMELNRLRSD |
| | | | DTAVYYCARDRTFYFDYWGQGTLVTVSS |
| 1196 | ATX-P-3433 | VH aa | QVQLVQSGAEVKKPGASVKVSCKASGYTFTDYYMHWVRQAPGQG |
| | | | LEWMGWINPNNGGTDYAQKFQGRVTMTRDTSISTAYMELHRLRSD |
| | | | DTAVYFCARGVYDVVVPTAIVNWEDPWGQGTLVTVSS |
| 1197 | ATX-P-3438 | VH aa | QVQLQESGPGLVKPSGTLSLTCAVSGDSISNDYWWNWVRQPPGKGL |
| | | | EWIGEIYHSGHTNYNPSLKSRVTISVDKSKNQLSLKLTSVNAADTAV |
| | | | YYCARGGTEITGYMDVWGKGTTVTVSS |
| 1198 | ATX-P-3441 | VH aa | QVQLQESGPGLVKPSGTLSLTCAVSGASIRSGHWWSWVRQPPGKGL |
| | | | EWIGEIYHSGNTNYNPSLKSRVTLSVDKSKNQFSLKLSSVTAADTAV |
| | | | YYCARGHYGDYVVYFDYWGQGTLVTVSS |
| 1199 | ATX-P-3446 | VH aa | QVQLQESGPGLVKPSETLSLTCTVSGGSISSYYWSWIRQPPGKGLEWI |
| | | | GYVYYSGYTKYNPSLKSRVTISIDASKNQFSLKLTSVTAADTAVYYC |
| | | | ATVESKWNYGPFAIWGQGTMVTVSS |
| 1200 | ATX-P-3452 | VH aa | QVQLVQSGAEVKKPGASVKVSCKASGYTFTGYYMNWVRQAPGQG |
| | | | LEWMGWINPNSGATNYAQKFQGRVTMTRDTSISTDYMELSRLRSD |
| | | | DTAVYYCARDGAKWNHEGYYFDYWGQGTLVTVSS |

TABLE 8
Anti-C3 antibodies (VL sequences)

| SEQ ID | Antibody | | |
|---|---|---|---|
| NO: | Name: | Descriptor: | Sequence: |
| 1201 | ATX-P-3360 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1202 | ATX-P-3362 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1203 | ATX-P-3363 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1204 | ATX-P-3365 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1205 | ATX-P-3367 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1206 | ATX-P-3369 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1207 | ATX-P-3370 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1208 | ATX-P-3371 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1209 | ATX-P-3373 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1210 | ATX-P-3375 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1211 | ATX-P-3378 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1212 | ATX-P-3382 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1213 | ATX-P-3383 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1214 | ATX-P-3384 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1215 | ATX-P-3386 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1216 | ATX-P-3387 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1217 | ATX-P-3389 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1218 | ATX-P-3391 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1219 | ATX-P-3392 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1220 | ATX-P-3395 | VL nt | GACATCCAGATGACCCAGTCTCCTTCCACCCTGTCTGCATCTGTA |
| | | | GGAGACAGCGTCACCATCACTTGCCGGGCCAGTCAGAATATTATT |
| | | | ACCTGGTTGGCTTGGTATCAGCAGAAACCAGGGAAAGCCCCTAA |
| | | | CCTCCTGATCTATAAGGCGTCTAGTTTAGAAAGTGGGGTCCCATC |
| | | | AAGGTTCAGCGGCAGTGGATCTGGGACAGAATTCACTCTCACCAT |
| | | | CAGCAGCCTGCAGCCTGATGATTTTGCAACTTATTACTGCCAACA |
| | | | GTATAGTAGTTATTCGTACACTTTTGGCCAGGGGACCAAGCTGGA |
| | | | GATCAAA |
| 1221 | ATX-P-3396 | VL nt | GACATCCAGATGACCCAGTCTCCTTCCACCCTGTCTGCATCTGTA |
| | | | GGAGACAGCGTCACCATCACTTGCCGGGCCAGTCAGAATATTATT |
| | | | ACCTGGTTGGCTTGGTATCAGCAGAAACCAGGGAAAGCCCCTAA |
| | | | CCTCCTGATCTATAAGGCGTCTAGTTTAGAAAGTGGGGTCCCATC |
| | | | AAGGTTCAGCGGCAGTGGATCTGGGACAGAATTCACTCTCACCAT |
| | | | CAGCAGCCTGCAGCCTGATGATTTTGCAACTTATTACTGCCAACA |
| | | | GTATAGTAGTTATTCGTACACTTTTGGCCAGGGGACCAAGCTGGA |
| | | | GATCAAA |
| 1222 | ATX-P-3397 | VL nt | GACATCCAGATGACCCAGTCTCCTTCCACCCTGTCTGCATCTGTA |
| | | | GGAGACAGCGTCACCATCACTTGCCGGGCCAGTCAGAATATTATT |
| | | | ACCTGGTTGGCTTGGTATCAGCAGAAACCAGGGAAAGCCCCTAA |
| | | | CCTCCTGATCTATAAGGCGTCTAGTTTAGAAAGTGGGGTCCCATC |
| | | | AAGGTTCAGCGGCAGTGGATCTGGGACAGAATTCACTCTCACCAT |
| | | | CAGCAGCCTGCAGCCTGATGATTTTGCAACTTATTACTGCCAACA |
| | | | GTATAGTAGTTATTCGTACACTTTTGGCCAGGGGACCAAGCTGGA |
| | | | GATCAAA |
| 1223 | ATX-P-3305 | VL nt | GACATCCAGATGACCCAGTCTCCTTCCACCCTGTCTGCATCTGTA |
| | | | GGAGACAGCGTCACCATCACTTGCCGGGCCAGTCAGAATATTATT |
| | | | ACCTGGTTGGCTTGGTATCAGCAGAAACCAGGGAAAGCCCCTAA |
| | | | CCTCCTGATCTATAAGGCGTCTAGTTTAGAAAGTGGGGTCCCATC |
| | | | AAGGTTCAGCGGCAGTGGATCTGGGACAGAATTCACTCTCACCAT |
| | | | CAGCAGCCTGCAGCCTGATGATTTTGCAACTTATTACTGCCAACA |
| | | | GTATAGTAGTTATTCGTACACTTTTGGCCAGGGGACCAAGCTGGA |
| | | | GATCAAA |
| 1224 | ATX-P-3306 | VL nt | GACATCCAGATGACCCAGTCTCCTTCCACCCTGTCTGCATCTGTA |
| | | | GGAGACAGCGTCACCATCACTTGCCGGGCCAGTCAGAATATTATT |
| | | | ACCTGGTTGGCTTGGTATCAGCAGAAACCAGGGAAAGCCCCTAA |
| | | | CCTCCTGATCTATAAGGCGTCTAGTTTAGAAAGTGGGGTCCCATC |
| | | | AAGGTTCAGCGGCAGTGGATCTGGGACAGAATTCACTCTCACCAT |
| | | | CAGCAGCCTGCAGCCTGATGATTTTGCAACTTATTACTGCCAACA |
| | | | GTATAGTAGTTATTCGTACACTTTTGGCCAGGGGACCAAGCTGGA |
| | | | GATCAAA |
| 1225 | ATX-P-3307 | VL nt | GACATCCAGATGACCCAGTCTCCTTCCACCCTGTCTGCATCTGTA |
| | | | GGAGACAGCGTCACCATCACTTGCCGGGCCAGTCAGAATATTATT |
| | | | ACCTGGTTGGCTTGGTATCAGCAGAAACCAGGGAAAGCCCCTAA |
| | | | CCTCCTGATCTATAAGGCGTCTAGTTTAGAAAGTGGGGTCCCATC |
| | | | AAGGTTCAGCGGCAGTGGATCTGGGACAGAATTCACTCTCACCAT |
| | | | CAGCAGCCTGCAGCCTGATGATTTTGCAACTTATTACTGCCAACA |
| | | | GTATAGTAGTTATTCGTACACTTTTGGCCAGGGGACCAAGCTGGA |
| | | | GATCAAA |
| 1226 | ATX-P-3308 | VL nt | GACATCCAGATGACCCAGTCTCCTTCCACCCTGTCTGCATCTGTA |
| | | | GGAGACAGCGTCACCATCACTTGCCGGGCCAGTCAGAATATTATT |
| | | | ACCTGGTTGGCTTGGTATCAGCAGAAACCAGGGAAAGCCCCTAA |
| | | | CCTCCTGATCTATAAGGCGTCTAGTTTAGAAAGTGGGGTCCCATC |
| | | | AAGGTTCAGCGGCAGTGGATCTGGGACAGAATTCACTCTCACCAT |
| | | | CAGCAGCCTGCAGCCTGATGATTTTGCAACTTATTACTGCCAACA |
| | | | GTATAGTAGTTATTCGTACACTTTTGGCCAGGGGACCAAGCTGGA |
| | | | GATCAAA |
| 1227 | ATX-P-3310 | VL nt | GACATCCAGATGACCCAGTCTCCTTCCACCCTGTCTGCATCTGTA |
| | | | GGAGACAGCGTCACCATCACTTGCCGGGCCAGTCAGAATATTATT |
| | | | ACCTGGTTGGCTTGGTATCAGCAGAAACCAGGGAAAGCCCCTAA |
| | | | CCTCCTGATCTATAAGGCGTCTAGTTTAGAAAGTGGGGTCCCATC |
| | | | AAGGTTCAGCGGCAGTGGATCTGGGACAGAATTCACTCTCACCAT |
| | | | CAGCAGCCTGCAGCCTGATGATTTTGCAACTTATTACTGCCAACA |
| | | | GTATAGTAGTTATTCGTACACTTTTGGCCAGGGGACCAAGCTGGA |
| | | | GATCAAA |
| 1228 | ATX-P-3312 | VL nt | GACATCCAGATGACCCAGTCTCCTTCCACCCTGTCTGCATCTGTA |
| | | | GGAGACAGCGTCACCATCACTTGCCGGGCCAGTCAGAATATTATT |
| | | | ACCTGGTTGGCTTGGTATCAGCAGAAACCAGGGAAAGCCCCTAA |
| | | | CCTCCTGATCTATAAGGCGTCTAGTTTAGAAAGTGGGGTCCCATC |
| | | | AAGGTTCAGCGGCAGTGGATCTGGGACAGAATTCACTCTCACCAT |
| | | | CAGCAGCCTGCAGCCTGATGATTTTGCAACTTATTACTGCCAACA |
| | | | GTATAGTAGTTATTCGTACACTTTTGGCCAGGGGACCAAGCTGGA |
| | | | GATCAAA |
| 1229 | ATX-P-3313 | VL nt | GACATCCAGATGACCCAGTCTCCTTCCACCCTGTCTGCATCTGTA |
| | | | GGAGACAGCGTCACCATCACTTGCCGGGCCAGTCAGAATATTATT |
| | | | ACCTGGTTGGCTTGGTATCAGCAGAAACCAGGGAAAGCCCCTAA |
| | | | CCTCCTGATCTATAAGGCGTCTAGTTTAGAAAGTGGGGTCCCATC |
| | | | AAGGTTCAGCGGCAGTGGATCTGGGACAGAATTCACTCTCACCAT |
| | | | CAGCAGCCTGCAGCCTGATGATTTTGCAACTTATTACTGCCAACA |
| | | | GTATAGTAGTTATTCGTACACTTTTGGCCAGGGGACCAAGCTGGA |
| | | | GATCAAA |
| 1230 | ATX-P-3314 | VL nt | GACATCCAGATGACCCAGTCTCCTTCCACCCTGTCTGCATCTGTA |
| | | | GGAGACAGCGTCACCATCACTTGCCGGGCCAGTCAGAATATTATT |
| | | | ACCTGGTTGGCTTGGTATCAGCAGAAACCAGGGAAAGCCCCTAA |
| | | | CCTCCTGATCTATAAGGCGTCTAGTTTAGAAAGTGGGGTCCCATC |
| | | | AAGGTTCAGCGGCAGTGGATCTGGGACAGAATTCACTCTCACCAT |
| | | | CAGCAGCCTGCAGCCTGATGATTTTGCAACTTATTACTGCCAACA |
| | | | GTATAGTAGTTATTCGTACACTTTTGGCCAGGGGACCAAGCTGGA |
| | | | GATCAAA |
| 1231 | ATX-P-3319 | VL nt | GCCATCCAGATGACCCAGTCTCCATCCTCCCTGTCTGCATCTGTA |
| | | | GGAGACAGAGTCACCATCACTTGCCGGGCAAGTCAGGGCATTAG |
| | | | AAATGATTTAGGCTGGTATCAGCAGAAACCAGGGAAAGCCCCTA |
| | | | AGCTCCTGATCTATGCTGCATCCAGTTTACAAAGTGGGGTCCCAT |
| | | | CAAGGTTCAGCGGCAGTGGATCTGGCACAGATTTCACTCTCACCA |
| | | | TCAGCAGCCTGCAGCCTGAAGATTTTGCAACTTATTACTGTCTAC |
| | | | AAGATTACAATTACCCGTGGACGTTCGGCCAAGGGACCAAGGTG |
| | | | GAAATCAAA |
| 1232 | ATX-P-3321 | VL nt | GACATCGTGATGACCCAGTCTCCAGACTCCCTGGCTGTGTCTCTG |
| | | | GGCGAGAGGGCCACCATCAACTGCAAGTCCAGCCAGACTGTTTT |
| | | | GTACAGGTCCAACAATAAGAACTACTTAGCTTGGTATCAGCAGA |
| | | | AACCAGGACAGCCTCCTAAGCTGCTCATTTACTGGGCATCTACCC |
| | | | GGGAATCCGGGGTCCCTGACCGATTCAGTGGCAGCGGGTCTGGG |
| | | | ACAGATTTCACTCTCACCATCAGCAGCCTGCAGGCTGAAGATGTG |
| | | | GCAGTTTATTACTGTCAGCAATATTATAGTACTCCGTGGACGTTC |
| | | | GGCCAAGGGACCAAGGTGGAAATCAAA |
| 1233 | ATX-P-3328 | VL nt | GCCATCCAGATGACCCAGTCTCCATCCTCCCTGTCTGCTTCTGTAG |
| | | | GAGACAGAGTCACCATCACTTGCCGGGCAAGTCAGGGCATTAGA |
| | | | AATGATTTAGGCTGGTATCAGCAGAATCCCGGGAAAGCCCCTAA |
| | | | GCTCCTGATTTATGCTGCATCCAATTTACAAAGTGGGGTCCCATC |
| | | | AAGGTTCAGCGGCAGTGGATCTGGCACAGATTTCACTCTCACCAT |
| | | | CAGCAGCCTGCAGCCTGAAGATTTTGTTACTTATTACTGTCTACA |
| | | | AGATTACAATTGGCCGTGGACGTTCGGCCAAGGGACACGACTGG |
| | | | AGATTAAA |
| 1234 | ATX-P-3330 | VL nt | GACATCCAGATGACCCAGTCTCCATCCTCCCTGTCTGCATCTGTA |
| | | | GGAGACAGAGTCACCATCACTTGCCGGGCAAGTCAGAGCATTAG |
| | | | CAACTATTTAAATTGGTATCAACAGAAACCAGAGAAAGCCCCTA |
| | | | AGTTCCTGATCTATGCTGCATCCAGTTTGCAAAGTGGGGTCCCAT |
| | | | CAAGGTTCAGTGGCAGTGGATCTGGGACAGATTTCACTCTCACCA |
| | | | TCAGCAGTCTGCAACCTGAAGATTTTGCAACTTACTACTGTCAAC |
| | | | AGAGTTACAGTTCCCCGCTCACTTTCGGCGGAGGGACCAAGGTGG |
| | | | AGATCAAA |
| 1235 | ATX-P-3331 | VL nt | GAAATTGTGTTGACTCAGTCTCCAGACTTTCAGTCTGTGACTCCA |
| | | | AAGGAGAAAGTCACCATCACCTGCCGGGCCAGTCAGAGCATTGG |
| | | | TAGTAACTTACACTGGTTCCAGCAGAAACCAGATCAGTCTCCAAA |
| | | | ACTCCTCATCAAGTATGCTTCCCAGTCCTTCTCAGGGGTCCCCTCG |
| | | | AGGTTCAGTGGCAGTGGATCTGGGACAGATTTCACCCTCACCATC |
| | | | AATAGCCTGGAAGCTGAAGATGCTGCAATGTATTACTGTCATCAG |
| | | | AGTAGTAGTTTACCTCGGACGTTCGGCCAAGGGACCAAGGTGGA |
| | | | AATCAAA |
| 1236 | ATX-P-3335 | VL nt | GCCATCCAGATGACCCAGTCTCCATCCTCCCTGTCTGCATCTGTA |
| | | | GGAGACAGAGTCACCATCACTTGCCGGGCAAGTCAGGGCATTAG |
| | | | AAATGATTTAGGCTGGTATCAGCAGAAACCAGGGAAAGCCCCTA |
| | | | AGCTCCTGATCTATGCTGCATCCAGTTTACAAAGTGGGGTCCCAT |
| | | | CAAGGTTCAGCGGCAGTGGATCTGGCACAGATTTCACTCTCACCA |
| | | | TCAGCAGCCTGCAGCCTGAAGATTTTGCATCTTATTACTGTCTAC |
| | | | AAGATTACAATTGGCCGTACACTTTTGGCCAGGGGACCAAGCTGG |
| | | | AGATCAAA |
| 1237 | ATX-P-3336 | VL nt | GAAATTGTGTTGACGCAGTCTCCAGGCACCCTGTCTTTGTCTCCA |
| | | | GGGGAAAGAGCCACCCTCTCCTGCAGGGCCAGTCAGAGTGTTAG |
| | | | AAGCAGCTACTTTGCCTGGTACCAGCAGAAACCTGGCCAGGCTCC |
| | | | CAGGCTCCTCATCTATGGTGCATCCAGCAGGGCCACTGGCATCCC |
| | | | AGACAGGTTCAGTGGCAGTGGGTCTGGGACAGACTTCACTCTCAC |
| | | | CATCAGCAGACTGGAGCCTGAAGATTTTGCAGTGTATTACTGTCA |
| | | | GCAGTATGGTAGCTCACCTCGGACGTTCGGCCAAGGGACCAAGG |
| | | | TGGAAATCAAA |
| 1238 | ATX-P-3337 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTACCC |
| | | | GGGAATCCGGGGTCCCTGACCGATTCAGTGGCAGCGGGTCTGGG |
| | | | ACAGATTTCACTCTCACCATCAGCAGCCTGCAGGCTGAAGATGTG |
| | | | GCAGTTTATTACTGTCAGCAATATTTTGGTACTCCTCGGACGTTCG |
| | | | GCCAAGGGACCAAGGTGGAAATCAAA |
| 1239 | ATX-P-3340 | VL nt | GAAATTGTGTTGACACAGTCTCCAGCCACCCTGTCTTTGTCTCCA |
| | | | GGGGAAAGAGCCACCCTCTCCTGCAGGGCCAGTCAGAGTGTTAG |
| | | | CAGCTACTTAGCCTGGTACCAACAGAAACCTGGCCAGGCTCCCAG |
| | | | GCTCCTCATCTATGATGCATCCAACAGGGCCACTGGCATCCCAGC |
| | | | CAGGTTCAGTGGCAGTGGGTCTGGGACAGACTTCACTCTCACCAT |
| | | | CAGCAGCCTGCAGTCTGAAGATTTTGCAGTTTATTACTGTCAGCA |
| | | | GCGTAGCAATTGGCCGCTCACTTTCGGCGGAGGGACCAAGGTGG |
| | | | AGATCAAA |
| 1240 | ATX-P-3342 | VL nt | GACATCCAGATGACCCAGTCTCCATCCTCCCTGTCTGCATCTGTA |
| | | | GGAGACAGAGTCACCATCACTTGCCGGGCAAGTCAGACCATTAG |
| | | | GAGCTATTTAAATTGGTATCAGCAGAAACCAGGGAAAGCCCCTA |
| | | | AGCTCCTGATCTATGCTGCATCCAGTTTGCAAAGTGGGGTCCCAT |
| | | | CAAGGTTCAGTGGCAGTGGATCTGGGACAAATTTCACTCTCACCA |
| | | | TCAGCAGTCTGCAACCTGAAGATTTTGCAACTTACTCCTGTCAAC |
| | | | AGAGTTACACTACCCCGCTCACTTTCGGCGGAGGGACCAAGGTG |
| | | | GAGATCAAA |
| 1241 | ATX-P-3344 | VL nt | GAAATTGTGTTGACGCAGTCTCCAGGCACCCTGTCTTTGTCTCCA |
| | | | GGGGAAAGAGCCACCCTCTCCTGCAGGGCCAGTCAGAGTATTAG |
| | | | CAGCAGCTACTTAGCCTGGTACCAGCGGGAACCTGGCCAGGCTCC |
| | | | CAGGCTCCTCATCTATGGTGCATCCAGCAGGGCCACTGGCATCCC |
| | | | AGACAGGTTCAGTGGCAGTGGGTCTGGGACAGACTTCACTCTCAC |
| | | | CATCAGCAGACTGGAGCCTGAAGATTTTGTAGTGTATTACTGTCA |
| | | | ACAATATGGTAGCTCACCGATCACCTTCGGCCAAGGGACACGACT |
| | | | GGAGATTAAA |
| 1242 | ATX-P-3347 | VL nt | GAAATTGTGTTGACGCAGTCTCCAGGCACCCTGTCTTTGTCTCCA |
| | | | GGGGAAAGAGCCACCCTCTCCTGCAGGGCCAGTCAGAGTGTTCG |
| | | | CAGCAGCTACTTAGCCTGGTACCAGCAGAAACCTGGCCAGGCTCC |
| | | | CAGGCTCCTCATCTATGGTGCATCCAGCAGGGCCACTGGCATCCC |
| | | | AGACAGGTTCAGTGGCAGTGGGTCTGGGACAGACTTCACTCTCAC |
| | | | CTTCAGCAGACTGGAGCCTGAAGATTTTGCAGTGTATTACTGTCA |
| | | | GCAGTATGGTAGCTCACCGTACACTTTTGGCCAGGGGACCAAGCT |
| | | | GGAGATCAAA |
| 1243 | ATX-P-3348 | VL nt | GACATCCAGATGACCCAGTCTCCATCCTCCCTGTCTGCATCTGTA |
| | | | GGAGACAGAGTCACCATCACTTGCCAGGCGAGTCAGGACATTAG |
| | | | CAACTATTTAAATTGGTATCAGCAGAAACCAGGGAAAGCCCCTA |
| | | | AGCTCCTGATCTACGATGCATCCAATTTGGAAACAGGGGTCCCAT |
| | | | CAAGGTTCAGTGGAAGTGGATCTGGGACAGATTTTACTTTCACCA |
| | | | TCAGCAGCCTGCAGCCTGAAGATTTTGCAACTTACTATTGTCAAC |
| | | | AGGCTAACAGTTTCCCGCTCACTTTCGGCGGAGGGACCAAGGTGG |
| | | | AGATCAAA |
| 1244 | ATX-P-3349 | VL nt | GAAATTGTGTTGACGCAGTCTCCAGGCACCCTGTCTTTGTCTCCA |
| | | | GGGGAAAGAGCCACCCTCTCCTGCAGGGCCAGTCAGAGTGTTAG |
| | | | CAGCAGCTACTTAGCCTGGTACCAGCAGAAACCTGGCCAGGCTCC |
| | | | CAGGCTCCTCATCTATGGTGCATCCAGCAGGGCCACTGGCATCCC |
| | | | AGACAGGTTCAGTGGCAGTGGGTCTGGGACAGACTTCACTCTCAC |
| | | | CATCAGCAGACTGGAGCCTGAAGATTTTGCAGTGTATTACTGTCA |
| | | | GCAGTATGGTAGCTCACCATTCACTTTCGGCCCTGGGACCAAAGT |
| | | | GGATATCAAA |
| 1245 | ATX-P-3350 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTACCC |
| | | | GGGAATCCGGGGTCCCTGACCGATTCAGTGGCAGCGGGTCTGGG |
| | | | ACAGATTTCACTCTCACCATCAGCAGCCTGCAGGCTGAAGATGTG |
| | | | GCAGTTTATTACTGTCAGCAATATTATAGTACTCCGTGGACGTTC |
| | | | GGCCAAGGGACACGACTGGAGATTAAA |
| 1246 | ATX-P-3351 | VL nt | GACATCCAGATGACCCAGTCTCCATCCTCCCTGTCTGCATCTGTA |
| | | | GGAGACAGAGTCACCATCACTTGCCGGGCGAGTCAGGACATTAG |
| | | | CAACTATTTAAATTGGTATCAGCAGAAACCAGGGAAAGCCCCTA |
| | | | AGCTCCTGATCTATGCTGCATCCAGTTTGGAAAGTGGGGTCCCAT |
| | | | CAAGGTTCAGTGGCAGTGGATCTGGGACAGATTTCACTCTCACCA |
| | | | TCAGCAGTCTGCAACCTGAAGATTTTGCAACTTACTACTGTCAAC |
| | | | AGAGTTACAGTACCCCTACTTTCGGCGGAGGGACCAAGGTGGAG |
| | | | ATCAAA |
| 1247 | ATX-P-3353 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGGTCCAACAATAAGAACTACTTAGCTTGGTACCAGCAGA |
| | | | AACCAGGCCAGCCTCCTAAACTGCTCATTTACTGGGCATCTACCC |
| | | | GGGAATCCGGGGTCCCTGACCGATTCAGTGGCAGCGGGTCTGGG |
| | | | ACAGATTTCACTCTCACCATCAGCAGCCTGCAGGCTGAAGATGTG |
| | | | GCAGTTTATTACTGTCAGCAATATTATGGTACTCCTCGGACGTTC |
| | | | GGCCAAGGGACCAAGGTGGAAATCAAA |
| 1248 | ATX-P-3354 | VL nt | GACATCCAGATGACCCAGTCTCCATCCTCCCTGTCTGCATCTGTA |
| | | | GGAGACAGAGTCACCATCACTTGCCAGGCGAGTCAGGACATTAA |
| | | | GAACTGTTTAAATTGGTATCAGCAGAAACCAGGGAAAGCCCCTA |
| | | | AGCTCCTGATCTACGATGCATCCAATTTGGAAACAGGGGTCCCAT |
| | | | CAAGGTTCAGTGGAAGTGGATCTGGGACAGATTTTACTTTCACCA |
| | | | TCAGCAGCCTGCAGCCTGAAGATATTGCAACATATTACTGTCAAC |
| | | | AGTTTGATAATCTCCCTCTCACTTTCGGCGGAGGGACCAAGGTGG |
| | | | AGATCAAA |
| 1249 | ATX-P-3355 | VL nt | GACATCGTGATGACCCAGTCTCCAGACTCCCTGGCTGTGTCTCTG |
| | | | GGCGAGAGGGCCACCATCAACTGCAAGTCCAGCCAGACTATTTT |
| | | | ATACAGGTCCAACATTAAGAACTACTTAGCTTGGTACCAGCAGAA |
| | | | ACCAGGACAGCCTCCTAAACTGCTCATTTCCTGGGCATCTACCCG |
| | | | GGAATCCGGGGTCCCTGACCGATTCAGTGGCAGCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCGGACGTTCGGCC |
| | | | AGGGGACCAAGCTGGAGATCAAA |
| 1250 | ATX-P-3356 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTACCC |
| | | | GGGAATCCGGGGTCCCTGACCGATTCAGTGGCAGCGGGTCTGGG |
| | | | ACAGATTTCACTCTCACCATCAGCAGCCTGCAGGCTGAAGATGTG |
| | | | GCAGTTTATTACTGTCAGCAATATTATAGTACTCCTCGGACGTTC |
| | | | GGCCAAGGGACCAAGGTGGAAATCAAA |
| 1251 | ATX-P-3358 | VL nt | GACATCCAGATGACCCAGTCTCCTTCCACCCTGTCTGCATCTGTA |
| | | | GGAGACAGAGTCACTATCACTTGTCGGGCGAGTCAGGGTATTAG |
| | | | CAGCTGGTTAGCCTGGTATCAGCAGAAACCAGGGAAAGCCCCTA |
| | | | AACTCCTGATCTATGCTGCATCCAGTTTGCAGAGTGGGGTCCCAT |
| | | | CAAGGTTCAGCGGCAGTGGATCTGGGACAGATTTCACTCTCACCA |
| | | | TCAGCAGCCTGCAGCCTGAAGATTTTGCAACTTACTATTGTCAAC |
| | | | AGGCTAACAGTTTCCCTCTCACTTTCGGCGGAGGGACCAAGGTGG |
| | | | AGATCAAA |
| 1252 | ATX-P-3359 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTACCC |
| | | | GGGAATCCGGGGTCCCTGACCGATTCAGTGGCAGCGGGTCTGGG |
| | | | ACAGATTTCACTCTCACCATCAGCAGCCTGCAGGCTGAAGATGTG |
| | | | GCAGTTTATTACTGTCAGCAATATTATAGTACTCCTCCGACGTTCG |
| | | | GCCAAGGGACCAAGGTGGAAATCAAA |
| 1253 | ATX-P-3362 | VL nt | GACATCCAGATGACCCAGTCTCCATCCTCCCTGTCTGCATCTGTA |
| | | | GGAGACAGAGTCACCATCACTTGCCGGGCAAGTCAGAGCATTAG |
| | | | CAGCTATTTAAATTGGTATCAGCAGAAACCAGGGAAAGCCCCTA |
| | | | AGCTCCTGATCTATGCTGCATCCAGTTTGCAAAGTGGGGTCCCAT |
| | | | CAAGGTTCAGTGGCAGTGGATCTGGGACAGATTTCACTCTCACCA |
| | | | TCAGCAGTCTGCAACCTGAAGATTTTGCAACTTACTACTGTCAAC |
| | | | AGAGTTACAGTACCCCGTACACTTTTGGCCAGGGGACCAAGCTGG |
| | | | AGATCAAA |
| 1254 | ATX-P-3363 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTACCC |
| | | | GGGAATCCGGGGTCCCTGACCGATTCAGTGGCAGCGGGTCTGGG |
| | | | ACAGATTTCACTCTCACCATCAGCAGCCTGCAGGCTGAAGATGTG |
| | | | GCAGTTTATTACTGTCAGCAATTTTATGATACTCCTCGGACGTTCG |
| | | | GCCAAGGGACCAAGGTGGAAATCAAA |
| 1255 | ATX-P-3366 | VL nt | GACATCCAGATGACCCAGTCTCCATCCTCCCTGTCTGCATCTGTA |
| | | | GGAGACAGAGTCACCATCACTTGCCGGGCGAGTCAGGGCATTAG |
| | | | CAATTATTTAGCCTGGTATCAGCAGAAACCAGGGAAAGTTCCTAA |
| | | | GCTCCTGATCTATGTTGCATCCACTTTGCAATCAGGGGTCCCATCT |
| | | | CGGTTCAGTGGCAGTGGATCTGGGACAGATTTCACTCTCACCATC |
| | | | AGCAGCCTGCAGCCTGAAGATGTTGCAACTTATTACTGTCAAAAT |
| | | | TATATCAGTGCCCCGCTCACTTTCGGCGGAGGGACCAAGGTGGAG |
| | | | ATCAAA |
| 1256 | ATX-P-3367 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTACCC |
| | | | GGGAATCCGGGGTCCCTGACCGATTCAGTGGCAGCGGGTCTGGG |
| | | | ACAGATTTCACTCTCACCATCAGCAGCCTGCAGGCTGAAGATGTG |
| | | | GCAGTTTATTACTGTCAGCAATATTATAGTACTCCGTGGACGTTC |
| | | | GGCCAAGGGACCAAGGTGGAAATCAAA |
| 1257 | ATX-P-3368 | VL nt | GAAATTGTGTTGACGCAGTCTCCAGGCACCCTGTCTTTGTCTCCA |
| | | | GGGGAAAGAGCCACCCTCTCCTGCAGGGCCAGTCAGAGTGTTAG |
| | | | CAGCAGCTACTTAGCCTGGTACCAGCAGAAACCTGGCCAGGCTCC |
| | | | CAGGCTCCTCATCTATGGTGCATCCAGCAGGGCCACTGGCATCCC |
| | | | AGACAGGTTCAGTGGCAGTGGGTCTGGGACAGACTTCACTCTCAC |
| | | | CATCAGCAGACTGGAGCCTGAAGATTTTGCAGTGTATTACTGTCA |
| | | | GCAGTATGGTAGCTCACCGTACACTTTTGGCCAGGGGACCAAGCT |
| | | | GGAGATCAAA |
| 1258 | ATX-P-3369 | VL nt | GACATCCAGATGACCCAGTCTCCATCCTCCCTGTCTGCATCTGTA |
| | | | GGAGACAGAGTCAACATCACTTGCCGGGCGAGTCAGGGCATTAG |
| | | | CAATTATTTAGCCTGGTATCAGCAGAAACCAGGGAAAGTTCCTAA |
| | | | GCTCCTGATCTATGCTGCATCCACTTTGCAATCAGGGGTCCCATCT |
| | | | CGTTTCAGTGGCAGTGGATCTGGGACAGATTTCACTCTCACCATC |
| | | | AGCAGCCTGCAGCCTGAAGATGTTGCAAGTTATTTCTGTCAAAAA |
| | | | TATATCAGTGCCCCGCTCACTTTCGGCGGAGGGACCAAGGTGGAG |
| | | | ATCAAA |
| 1259 | ATX-P-3370 | VL nt | GACATCCAGATGACCCAGTCTCCATCCTCCCTGTCTGCATCTGTA |
| | | | GGAGACAGAGTCACCATCACTTGCCGGGCGAGTCAGGGCATTAG |
| | | | CAATTATTTAGCCTGGTATCAGCAGAAACCAGGGAAAGTTCCTAA |
| | | | GCTCCTGATCTATGCTGCATCCACTTTGCAATCAGGGGTCCCATCT |
| | | | CGGTTCAGTGGCAGTGGATCTGGGACAGATTTCACTCTCACCATC |
| | | | AGCAGCCTGCAGCCTGAAGACGTTGCAAGTTATTACTGTCAAGAT |
| | | | TATACCAGTGCCCCGCTCACTTTCGGCGGAGGGACCAAGGTGGA |
| | | | GATCAAA |
| 1260 | ATX-P-3371 | VL nt | GAAATTGTGTTGACGCAGTCTCCAGGCACCCTGTCTTTGTCTCCA |
| | | | GGGGAAAGAGCCACCCTCTCCTGCAGGGCCAGTCAGAGTGTTAG |
| | | | TGACAGCTACTTAGCCTGGTACCAGCAGGAACCTGGCCAGGCGC |
| | | | CCAGGCTCCTCATCTATGGTGCATCCAGCAGGGCCACTGGCATCC |
| | | | CAGACAGGTTCAGTGGCAGTGGGTCTGGGACAGACTTCACTCTCA |
| | | | CCATCAGCAGACTGGAGCCTGAAGATTTTGCATTATATTACTGTC |
| | | | AGCAATATGATTCCTCGCCATTCACTTTCGGCCCTGGGACCAAAG |
| | | | TGGATATCAAA |
| 1261 | ATX-P-3472 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTACCC |
| | | | GGGAATCCGGGGTCCCTGACCGATTCAGTGGCAGCGGGTCTGGG |
| | | | ACAGATTTCACTCTCACCATCAGCAGCCTGCAGGCTGAAGATGTG |
| | | | GCAGTTTATTACTGTCAGCAATATTATAGTACTCCGTACACTTTTG |
| | | | GCCAGGGGACCAAGCTGGAGATCAAA |
| 1262 | ATX-P-3361 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1263 | ATX-P-3366 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1264 | ATX-P-3376 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1265 | ATX-P-3377 | VL nt | GACATCGTGATGACCCAGTCTCCAGACTCCCTGGCTGTGTCTCTG |
| | | | GGCGAGAGGGCCACCATCAACTGCAAGTCCAGCCAGAGTGTTTT |
| | | | ATACAGCTCCAACAATAAGAACTACTTAGCTTGGTACCAGCAGA |
| | | | AACCAGGACAGCCTCCTAAGCTGCTCATTTACTGGGCATCTAACC |
| | | | GGGAATCCGGGGTCCCTGACCGATTCAGTGGCTCCGGGTCTGGGA |
| | | | CAGATTTCACTCTCACCATCAGCAGCCTGCAGGCTGAAGATGTGG |
| | | | CAGTTTATTACTGTCAGCAATATTATAGTACTCCGTACACTTTTGG |
| | | | CCAGGGGACCAAGCTGGAGATCAAA |
| 1266 | ATX-P-3418 | VL nt | GACATCCAGATGACCCAGTCTCCATCCTCCCTGTCTGCATCTGTA |
| | | | GGAGACAGAGTCACCATCACCTGCCGGGCAAGTCAGACCATTAG |
| | | | AAACTTTTTAAATTGGTATCAGCGGAAACCAGGGAAAGCCCCTA |
| | | | AGATCCTGATCTATGCTGCATCCAGTTTGCAAAGTGGGGTCCCAT |
| | | | CAAGGTTCAGTGGCAGTGGATCTGGGACAGATTTCACTCTCACCA |
| | | | TCAGCAGTCTGCAACCTGCAGATTTTGCAACTTACTACTGTCAAC |
| | | | AGACTTACAGTATCCCGATCACCTTCGGCCAAGGGACACGACTGG |
| | | | AGATTAAA |
| 1267 | ATX-P-3420 | VL nt | GACATCCAGATGACCCAGTCTCCATCCTCCCTGTCTGCATCTGTA |
| | | | GGAGACAGAGTCACCATCACTTGCCGGGCAAGTCAGAGCATTAG |
| | | | CAGGTATTTAAATTGGTATCAGCAGAAACCAGGGAAAGCCCCTA |
| | | | AACTCCTGCTCTATGCTGCATCCAGTTTGCAAAGTGGGGTCCCAT |
| | | | CAAAGTTCAGCGGCAGTGGATCTGGGACAGATTTCACTCTCACCA |
| | | | TCAGCAGCCTGCAACCTGAAGATTTTGCAACTTACTACTGTCAAC |
| | | | AGAGTTACAGTTCCCCGATCACCTTCGGCCAAGGGACACGACTGG |
| | | | AGATTAAA |
| 1268 | ATX-P-3426 | VL nt | GATATTGTGATGACTCAGTCTCCACTCTCCCTGCCCGTCACCCCTG |
| | | | GAGAGCCGGCCTCCATCTCCTGCAGGTCTAGTCAGAGCCTCCTGC |
| | | | ATAGTAATGGATACAACTATTTGGATTGGTACCTGCAGAAGCCAG |
| | | | GGCAGTCTCCACAGCTCCTGATCTATTTGGGTTCTAATCGGGCCT |
| | | | CCGGGGTCCCTGACAGGTTCAGTGGCAGTGGATCAGGCACAGAT |
| | | | TTTACACTGAAAATCAGCAGAGTGGAGGCTGAGGATGTTGGGGT |
| | | | TTATTACTGCATGCAAACTCTACAAACTCCTCCGACGTTCGGCCA |
| | | | AGGGACCAAGGTGGAAATCAAA |
| 1269 | ATX-P-3432 | VL nt | GACATCCAGTTGACCCAGTCTCCATCCTTCCTGTCTGCATCTGTAG |
| | | | GAGACAGACTCACCATCACTTGCCGGGCCAGTCAGGGCATTAGC |
| | | | AGTTATTTAGCCTGGTATCAGCAAAAACCAGGGAAAGCCCCTAA |
| | | | GCTCCTGATCTATACTGCATCCACTTTACAAAGTGGGGTCCCATC |
| | | | AAGGTTCAGCGGCAGTGGATCTGGGACAGAATTCACTCTCACAAT |
| | | | CAGCAGCCTGCAGCCTGAAGATTTTGCAACTTATTACTGTCAACA |
| | | | GCTTAATGGTTACCCTCCGACGTTCGGCCAAGGGACCAAGGTGGA |
| | | | AATCAAA |
| 1270 | ATX-P-3433 | VL nt | GACATCCAGATGACCCAGTCTCCATCCTCCCTGTCTGCATCTGTA |
| | | | GGAGACAGAGTCACCATCACTTGCCGGGCAAGTCAGAGCATTAC |
| | | | CAGCTATTTAAATTGGTATCAGCAGAAACCAGGGAAAGCCCCTA |
| | | | AGCTCCTGATCTTTGCTGCATCCAGTTTGCAAAGTGGGGTCCCAT |
| | | | CAAGGTTCAGTGGCAGTGGATCTGGGACAGATTTCACTCTCACCA |
| | | | TCAGCAGTCTGCAACCTGAAGATTTTGCAACTTACTACTGTCAAC |
| | | | AGAGTTACAGTACCCCTCTCACTTTCGGCGGAGGGACCAAGGTGG |
| | | | AGATCAAA |
| 1282 | ATX-P-3371 | VL aa | DIVMTQSPDSLAVSLGERATINCKSSQSVLYSSNNKNYLAWYQQKP |
| | | | GQPPKLLIYWASNRESGVPDRFSGSGSGTDFTLTISSLQAEDVAVYY |
| | | | CQQYYSTPYTFGQGTKLEIK |
| 1283 | ATX-P-3373 | VL aa | DIVMTQSPDSLAVSLGERATINCKSSQSVLYSSNNKNYLAWYQQKP |
| | | | GQPPKLLIYWASNRESGVPDRFSGSGSGTDFTLTISSLQAEDVAVYY |
| | | | CQQYYSTPYTFGQGTKLEIK |
| 1284 | ATX-P-3375 | VL aa | DIVMTQSPDSLAVSLGERATINCKSSQSVLYSSNNKNYLAWYQQKP |
| | | | GQPPKLLIYWASNRESGVPDRFSGSGSGTDFTLTISSLQAEDVAVYY |
| | | | CQQYYSTPYTFGQGTKLEIK |
| 1285 | ATX-P-3378 | VL aa | DIVMTQSPDSLAVSLGERATINCKSSQSVLYSSNNKNYLAWYQQKP |
| | | | GQPPELLIYWASNRESGVPDRFSGSGSGTDFTLTISSLQAEDVAVYY |
| | | | CQQYYSTPYTFGQGTKLEIK |
| 1286 | ATX-P-3382 | VL aa | DIVMTQSPDSLAVSLGERATINCKSSQSVLYSSNNKNYLAWYQQKP |
| | | | GQPPKLLIYWASNRESGVPDRFSGSGSGTDFTLTISSLQAEDVAVYY |
| | | | CQQYYSTPYTFGQGTKLEIK |
| 1287 | ATX-P-3383 | VL aa | DIVMTQSPDSLAVSLGERATINCKSSQSVLYSSNNKNYLAWYQQKP |
| | | | GQPPKLLIYWASNRESGVPDRFSGSGSGTDFTLTISSLQAEDVAVYY |
| | | | CQQYYSTPYTFGQGTKLEIK |
| 1288 | ATX-P-3384 | VL aa | DIVMTQSPDSLAVSLGERATINCKSSQSVLYSSNNKNYLAWYQQKP |
| | | | GQPPKLLIYWASNRESGVPDRFSGSGSGTDFTLTISSLQAEDVAVYY |
| | | | CQQYYSTPYTFGQGTKLEIK |
| 1289 | ATX-P-3386 | VL aa | DIVMTQSPDSLAVSLGERATINCKSSQSVLYSSNNKNYLAWYQQKP |
| | | | GQPPKLLIYWASNRESGVPDRFSGSGSGTDFTLTISSLQAEDVAVYY |
| | | | CQQYYSTPYTFGQGTKLEIK |
| 1290 | ATX-P-3387 | VL aa | DIVMTQSPDSLAVSLGERATINCKSSQSVLYSSNNKNYLAWYQQKP |
| | | | GQPPKLLIYWASNRESGVPDRFSGSGSGTDFTLTISSLQAEDVAVYY |
| | | | CQQYYSTPYTFGQGTKLEIK |
| 1291 | ATX-P-3389 | VL aa | DIVMTQSPDSLAVSLGERATINCKSSQSVLYSSNNKNYLAWYQQKP |
| | | | GQPPKLLIYWASNRESGVPDRFSGSGSGTDFTLTISSLQAEDVAVYY |
| | | | CQQYYSTPYTFGQGTKLEIK |
| 1292 | ATX-P-3391 | VL aa | DIVMTQSPDSLAVSLGERATINCKSSQSVLYSSNNKNYLAWYQQKP |
| | | | GQPPELLIYWASNRESGVPDRFSGSGSGTDFTLTISSLQAEDVAVYY |
| | | | CQQYYSTPYTFGQGTKLEIK |
| 1293 | ATX-P-3392 | VL aa | DIVMTQSPDSLAVSLGERATINCKSSQSVLYSSNNKNYLAWYQQKP |
| | | | GQPPKLLIYWASNRESGVPDRFSGSGSGTDFTLTISSLQAEDVAVYY |
| | | | CQQYYSTPYTFGQGTKLEIK |
| 1294 | ATX-P-3395 | VL aa | DIQMTQSPSTLSASVGDSVTITCRASQNIITWLAWYQQKPGKAPNLLI |
| | | | YKASSLESGVPSRFSGSGSGTEFTLTISSLQPDDFATYYCQQYSSYSY |
| | | | TFGQGTKLEIK |
| 1295 | ATX-P-3396 | VL aa | DIQMTQSPSTLSASVGDSVTITCRASQNIITWLAWYQQKPGKAPNLLI |
| | | | YKASSLESGVPSRFSGSGSGTEFTLTISSLQPDDFATYYCQQYSSYSY |
| | | | TFGQGTKLEIK |
| 1296 | ATX-P-3397 | VL aa | DIQMTQSPSTLSASVGDSVTITCRASQNIITWLAWYQQKPGKAPNLLI |
| | | | YKASSLESGVPSRFSGSGSGTEFTLTISSLQPDDFATYYCQQYSSYSY |
| | | | TFGQGTKLEIK |
| 1297 | ATX-P-3405 | VL aa | DIQMTQSPSTLSASVGDSVTITCRASQNIITWLAWYQQKPGKAPNLLI |
| | | | YKASSLESGVPSRFSGSGSGTEFTLTISSLQPDDFATYYCQQYSSYSY |
| | | | TFGQGTKLEIK |
| 1298 | ATX-P-3406 | VL aa | DIQMTQSPSTLSASVGDSVTITCRASQNIITWLAWYQQKPGKAPNLLI |
| | | | YKASSLESGVPSRFSGSGSGTEFTLTISSLQPDDFATYYCQQYSSYSY |
| | | | TFGQGTKLEIK |
| 1299 | ATX-P-3407 | VL aa | DIQMTQSPSTLSASVGDSVTITCRASQNIITWLAWYQQKPGKAPNLLI |
| | | | YKASSLESGVPSRFSGSGSGTEFTLTISSLQPDDFATYYCQQYSSYSY |
| | | | TFGQGTKLEIK |
| 1300 | ATX-P-3408 | VL aa | DIQMTQSPSTLSASVGDSVTITCRASQNIITWLAWYQQKPGKAPNLLI |
| | | | YKASSLESGVPSRFSGSGSGTEFTLTISSLQPDDFATYYCQQYSSYSY |
| | | | TFGQGTKLEIK |
| 1301 | ATX-P-3410 | VL aa | DIQMTQSPSTLSASVGDSVTITCRASQNIITWLAWYQQKPGKAPNLLI |
| | | | YKASSLESGVPSRFSGSGSGTEFTLTISSLQPDDFATYYCQQYSSYSY |
| | | | TFGQGTKLEIK |
| 1302 | ATX-P-3412 | VL aa | DIQMTQSPSTLSASVGDSVTITCRASQNIITWLAWYQQKPGKAPNLLI |
| | | | YKASSLESGVPSRFSGSGSGTEFTLTISSLQPDDFATYYCQQYSSYSY |
| | | | TFGQGTKLEIK |
| 1303 | ATX-P-3413 | VL aa | DIQMTQSPSTLSASVGDSVTITCRASQNIITWLAWYQQKPGKAPNLLI |
| | | | YKASSLESGVPSRFSGSGSGTEFTLTISSLQPDDFATYYCQQYSSYSY |
| | | | TFGQGTKLEIK |
| 1304 | ATX-P-3414 | VL aa | DIQMTQSPSTLSASVGDSVTITCRASQNIITWLAWYQQKPGKAPNLLI |
| | | | YKASSLESGVPSRFSGSGSGTEFTLTISSLQPDDFATYYCQQYSSYSY |
| | | | TFGQGTKLEIK |
| 1305 | ATX-P-3419 | VL aa | AIQMTQSPSSLSASVGDRVTITCRASQGIRNDLGWYQQKPGKAPKLL |
| | | | IYAASSLQSGVPSRFSGSGSGTDFTLTISSLQPEDFATYYCLQDYNYP |
| | | | WTFGQGTKVEIK |
| 1306 | ATX-P-3421 | VL aa | DIVMTQSPDSLAVSLGERATINCKSSQTVLYRSNNKNYLAWYQQKP |
| | | | GQPPKLLIYWASTRESGVPDRFSGSGSGTDFTLTISSLQAEDVAVYYC |
| | | | QQYYSTPWTFGQGTKVEIK |
| 1307 | ATX-P-3428 | VL aa | AIQMTQSPSSLSASVGDRVTITCRASQGIRNDLGWYQQNPGKAPKLL |
| | | | IYAASNLQSGVPSRFSGSGSGTDFTLTISSLQPEDFVTYYCLQDYNWP |
| | | | WTFGQGTRLEIK |
| 1308 | ATX-P-3430 | VL aa | DIQMTQSPSSLSASVGDRVTITCRASQSISNYLNWYQQKPEKAPKFLI |
| | | | YAASSLQSGVPSRFSGSGSGTDFTLTISSLQPEDFATYYCQQSYSSPLT |
| | | | FGGGTKVEIK |
| 1309 | ATX-P-3431 | VL aa | EIVLTQSPDFQSVTPKEKVTITCRASQSIGSNLHWFQQKPDQSPKLLIK |
| | | | YASQSFSGVPSRFSGSGSGTDFTLTINSLEAEDAAMYYCHQSSSLPRT |
| | | | FGQGTKVEIK |
| 1310 | ATX-P-3435 | VL aa | AIQMTQSPSSLSASVGDRVTITCRASQGIRNDLGWYQQKPGKAPKLL |
| | | | IYAASSLQSGVPSRFSGSGSGTDFTLTISSLQPEDFASYYCLQDYNWP |
| | | | YTFGQGTKLEIK |
| 1311 | ATX-P-3436 | VL aa | EIVLTQSPGTLSLSPGERATLSCRASQSVRSSYFAWYQQKPGQAPRLL |
| | | | IYGASSRATGIPDRFSGSGSGTDFTLTISRLEPEDFAVYYCQQYGSSPR |
| | | | TFGQGTKVEIK |
| 1312 | ATX-P-3437 | VL aa | DIVMTQSPDSLAVSLGERATINCKSSQSVLYSSNNKNYLAWYQQKP |
| | | | GQPPKLLIYWASTRESGVPDRFSGSGSGTDFTLTISSLQAEDVAVYYC |
| | | | QQYFGTPRTFGQGTKVEIK |
| 1313 | ATX-P-3440 | VL aa | EIVLTQSPATLSLSPGERATLSCRASQSVSSYLAWYQQKPGQAPRLLI |
| | | | YDASNRATGIPARFSGSGSGTDFTLTISSLQSEDFAVYYCQQRSNWPL |
| | | | TFGGGTKVEIK |
| 1314 | ATX-P-3442 | VL aa | DIQMTQSPSSLSASVGDRVTITCRASQTIRSYLNWYQQKPGKAPKLLI |
| | | | YAASSLQSGVPSRFSGSGSGTNFTLTISSLQPEDFATYSCQQSYTTPLT |
| | | | FGGGTKVEIK |
| 1315 | ATX-P-3444 | VL aa | EIVLTQSPGTLSLSPGERATLSCRASQSISSSYLAWYQREPGQAPRLLI |
| | | | YGASSRATGIPDRFSGSGSGTDFTLTISRLEPEDFVVYYCQQYGSSPIT |
| | | | FGQGTRLEIK |
| 1316 | ATX-P-3447 | VL aa | EIVLTQSPGTLSLSPGERATLSCRASQSVRSSYLAWYQQKPGQAPRL |
| | | | LIYGASSRATGIPDRFSGSGSGTDFTLTFSRLEPEDFAVYYCQQYGSS |
| | | | PYTFGQGTKLEIK |
| 1317 | ATX-P-3448 | VL aa | DIQMTQSPSSLSASVGDRVTITCQASQDISNYLNWYQQKPGKAPKLL |
| | | | IYDASNLETGVPSRFSGSGSGTDFTFTISSLQPEDFATYYCQQANSFPL |
| | | | TFGGGTKVEIK |
| 1318 | ATX-P-3449 | VL aa | EIVLTQSPGTLSLSPGERATLSCRASQSVSSSYLAWYQQKPGQAPRLL |
| | | | IYGASSRATGIPDRFSGSGSGTDFTLTISRLEPEDFAVYYCQQYGSSPF |
| | | | TFGPGTKVDIK |
| 1319 | ATX-P-3450 | VL aa | DIVMTQSPDSLAVSLGERATINCKSSQSVLYSSNNKNYLAWYQQKP |
| | | | GQPPKLLIYWASTRESGVPDRFSGSGSGTDFTLTISSLQAEDVAVYYC |
| | | | QQYYSTPWTFGQGTRLEIK |
| 1320 | ATX-P-3451 | VL aa | DIQMTQSPSSLSASVGDRVTITCRASQDISNYLNWYQQKPGKAPKLL |
| | | | IYAASSLESGVPSRFSGSGSGTDFTLTISSLQPEDFATYYCQQSYSTPT |
| | | | FGGGTKVEIK |
| 1321 | ATX-P-3453 | VL aa | DIVMTQSPDSLAVSLGERATINCKSSQSVLYRSNNKNYLAWYQQKP |
| | | | GQPPKLLIYWASTRESGVPDRFSGSGSGTDFTLTISSLQAEDVAVYYC |
| | | | QQYYGTPRTFGQGTKVEIK |
| 1322 | ATX-P-3454 | VL aa | DIQMTQSPSSLSASVGDRVTITCQASQDIKNCLNWYQQKPGKAPKLL |
| | | | IYDASNLETGVPSRFSGSGSGTDFTFTISSLQPEDIATYYCQQFDNLPL |
| | | | TFGGGTKVEIK |
| 1323 | ATX-P-3455 | VL aa | DIVMTQSPDSLAVSLGERATINCKSSQTILYRSNIKNYLAWYQQKPG |
| | | | QPPKLLISWASTRESGVPDRFSGSGSGTDFTLTISSLQAEDVAVYYCQ |
| | | | QYYSTRTFGQGTKLEIK |
| 1324 | ATX-P-3456 | VL aa | DIVMTQSPDSLAVSLGERATINCKSSQSVLYSSNNKNYLAWYQQKP |
| | | | GQPPKLLIYWASTRESGVPDRFSGSGSGTDFTLTISSLQAEDVAVYYC |
| | | | QQYYSTPRTFGQGTKVEIK |
| 1325 | ATX-P-3458 | VL aa | DIQMTQSPSTLSASVGDRVTITCRASQGISSWLAWYQQKPGKAPKLL |
| | | | IYAASSLQSGVPSRFSGSGSGTDFTLTISSLQPEDFATYYCQQANSFPL |
| | | | TFGGGTKVEIK |
| 1326 | ATX-P-3459 | VL aa | DIVMTQSPDSLAVSLGERATINCKSSQSVLYSSNNKNYLAWYQQKP |
| | | | GQPPKLLIYWASTRESGVPDRFSGSGSGTDFTLTISSLQAEDVAVYYC |
| | | | QQYYSTPPTFGQGTKVEIK |
| 1327 | ATX-P-3462 | VL aa | DIQMTQSPSSLSASVGDRVTITCRASQSISSYLNWYQQKPGKAPKLLI |
| | | | YAASSLQSGVPSRFSGSGSGTDFTLTISSLQPEDFATYYCQQSYSTPY |
| | | | TFGQGTKLEIK |
| 1328 | ATX-P-3463 | VL aa | DIVMTQSPDSLAVSLGERATINCKSSQSVLYSSNNKNYLAWYQQKP |
| | | | GQPPKLLIYWASTRESGVPDRFSGSGSGTDFTLTISSLQAEDVAVYYC |
| | | | QQFYDTPRTFGQGTKVEIK |
| 1329 | ATX-P-3466 | VL aa | DIQMTQSPSSLSASVGDRVTITCRASQGISNYLAWYQQKPGKVPKLL |
| | | | IYVASTLQSGVPSRFSGSGSGTDFTLTISSLQPEDVATYYCQNYISAPL |
| | | | TFGGGTKVEIK |
| 1330 | ATX-P-3467 | VL aa | DIVMTQSPDSLAVSLGERATINCKSSQSVLYSSNNKNYLAWYQQKP |
| | | | GQPPKLLIYWASTRESGVPDRFSGSGSGTDFTLTISSLQAEDVAVYYC |
| | | | QQYYSTPWTFGQGTKVEIK |
| 1331 | ATX-P-3468 | VL aa | EIVLTQSPGTLSLSPGERATLSCRASQSVSSSYLAWYQQKPGQAPRLL |
| | | | IYGASSRATGIPDRFSGSGSGTDFTLTISRLEPEDFAVYYCQQYGSSPY |
| | | | TFGQGTKLEIK |
| 1332 | ATX-P-3469 | VL aa | DIQMTQSPSSLSASVGDRVNITCRASQGISNYLAWYQQKPGKVPKLL |
| | | | IYAASTLQSGVPSRFSGSGSGTDFTLTISSLQPEDVASYFCQKYISAPL |
| | | | TFGGGTKVEIK |
| 1333 | ATX-P-3470 | VL aa | DIQMTQSPSSLSASVGDRVTITCRASQGISNYLAWYQQKPGKVPKLL |
| | | | IYAASTLQSGVPSRFSGSGSGTDFTLTISSLQPEDVASYYCQDYTSAP |
| | | | LTFGGGTKVEIK |
| 1334 | ATX-P-3471 | VL aa | EIVLTQSPGTLSLSPGERATLSCRASQSVSDSYLAWYQQEPGQAPRLL |
| | | | TYGASSRATGIPDRFSGSGSGTDFTLTISRLEPEDFALYYCQQYDSSPF |
| | | | TFGPGTKVDIK |
| 1335 | ATX-P-3472 | VL aa | DIVMTQSPDSLAVSLGERATINCKSSQSVLYSSNNKNYLAWYQQKP |
| | | | GQPPKLLIYWASTRESGVPDRFSGSGSGTDFTLTISSLQAEDVAVYYC |
| | | | QQYYSTPYTFGQGTKLEIK |
| 1336 | ATX-P-3361 | VL aa | DIVMTQSPDSLAVSLGERATINCKSSQSVLYSSNNKNYLAWYQQKP |
| | | | GQPPKLLIYWASNRESGVPDRFSGSGSGTDFTLTISSLQAEDVAVYY |
| | | | CQQYYSTPYTFGQGTKLEIK |
| 1337 | ATX-P-3366 | VL aa | DIVMTQSPDSLAVSLGERATINCKSSQSVLYSSNNKNYLAWYQQKP |
| | | | GQPPKLLIYWASNRESGVPDRFSGSGSGTDFTLTISSLQAEDVAVYY |
| | | | CQQYYSTPYTFGQGTKLEIK |
| 1338 | ATX-P-3376 | VL aa | DIVMTQSPDSLAVSLGERATINCKSSQSVLYSSNNKNYLAWYQQKP |
| | | | GQPPKLLIYWASNRESGVPDRFSGSGSGTDFTLTISSLQAEDVAVYY |
| | | | CQQYYSTPYTFGQGTKLEIK |
| 1339 | ATX-P-3377 | VL aa | DIVMTQSPDSLAVSLGERATINCKSSQSVLYSSNNKNYLAWYQQKP |
| | | | GQPPELLIYWASNRESGVPDRFSGSGSGTDFTLTISSLQAEDVAVYY |
| | | | CQQYYSTPYTFGQGTKLEIK |
| 1340 | ATX-P-3418 | VL aa | DIQMTQSPSSLSASVGDRVTITCRASQTIRNFLNWYQRKPGKAPKILI |
| | | | YAASSLQSGVPSRFSGSGSGTDFTLTISSLQPADFATYYCQQTYSIPIT |
| | | | FGQGTRLEIK |
| 1341 | ATX-P-3420 | VL aa | DIQMTQSPSSLSASVGDRVTITCRASQSISRYLNWYQQKPGKAPKLL |
| | | | LYAASSLQSGVPSKFSGSGSGTDFTLTISSLQPEDFATYYCQQSYSSPI |
| | | | TFGQGTRLEIK |
| 1342 | ATX-P-3426 | VL aa | DIVMTQSPLSLPVTPGEPASISCRSSQSLLHSNGYNYLDWYLQKPGQS |
| | | | PQLLIYLGSNRASGVPDRFSGSGSGTDFTLKISRVEAEDVGVYYCMQ |
| | | | TLQTPPTFGQGTKVEIK |
| 1343 | ATX-P-3432 | VL aa | DIQLTQSPSFLSASVGDRLTITCRASQGISSYLAWYQQKPGKAPKLLI |
| | | | YTASTLQSGVPSRFSGSGSGTEFTLTISSLQPEDFATYYCQQLNGYPP |
| | | | TFGQGTKVEIK |
| 1344 | ATX-P-3433 | VL aa | DIQMTQSPSSLSASVGDRVTITCRASQSITSYLNWYQQKPGKAPKLLI |
| | | | FAASSLQSGVPSRFSGSGSGTDFTLTISSLQPEDFATYYCQQSYSTPLT |
| | | | FGGGTKVEIK |
| 1345 | ATX-P-3438 | VL aa | DIVMTQSPDSLAVSLGERATINCKSSQSVLYSSNNKNYLAWYQQKP |
| | | | GQPPKLLIYWASTRESGVPDRFSGSGSGTDFTLTISSLQAEDVAVYYC |
| | | | QQYYSTPRTFGQGTKVEIK |
| 1346 | ATX-P-3441 | VL aa | DIQMTQSPSSLSASVGERVTITCRASQSISSYLNWYQQKPGKAPNLLI |
| | | | YAASSFQSGVPSRVSGSGSGTDFTLTISSLQPEDFATYYCQQSYSNPY |
| | | | TFGQGTKLEIK |
| 1347 | ATX-P-3446 | VL aa | EIVLTQSPGTLSLSPGERATLSCRASQSVSRSYLAWYQQKPGQAPRL |
| | | | LIYGASSRATGIPDRFSGSGSGTDFTLTISRLEPEDFAVYYCQQYGSSP |
| | | | WTFGQGTKVEIK |
| 1348 | ATX-P-3452 | VL aa | DIVMTQSPLSLPVTPGEPASISCRSSQILLHTNGYNYLDWYLQKPGQS |
| | | | PQLLIYLGSYRASGVPDRFSGSGSGTDFTLKISRVEAGDVGLYYCMQ |
| | | | ALQTPLIFGGGTKVEIK |

TABLE 9
Anti-C3 antibodies (HC IgG1 Fc sequences)

| SEQ | Antibody | | |
|---|---|---|---|
| ID NO: | Name: | Descriptor: | Sequence: |
| 1349 | ATX-P-3360 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCATTAATAACTGGTGGAGTTGGGTCCGCCAGCCCCCAGGG |
| | | | AAGGGGCTGGAGTGGCTTGGAAAAATCTATCATAGTGGGAAC |
| | | | ACCGACTACAACCCGTCCCTCAAGAGTCGAGTCACCATATCAG |
| | | | TAGACAAGTCCAAGAATCAGTTCTCCCTGAGGCTGAGTTCTGT |
| | | | GACCGCCGCGGACACGGCCGTATATTATTGTGCGAGCGGAAG |
| | | | CGATTACGATTTTTGGAGTGGTTATTTGGACTACTGGGGCCAG |
| | | | GGAACCCTGGTCACCGTCTCCTCAGCTAGCACTAAAGGGCCTT |
| | | | CTGTATTTCCCTTGGCCCCGTCCAGCAAATCGACCTCGGGAGG |
| | | | GACAGCCGCCCTGGGTTGCCTTGTGAAAGATTATTTCCCTGAG |
| | | | CCAGTTACCGTAAGTTGGAACAGTGGGGCGCTGACAAGTGGT |
| | | | GTGCACACGTTTCCTGCCGTCCTGCAATCATCGGGCTTGTATA |
| | | | GCCTCAGCTCTGTGGTCACTGTCCCAAGTTCATCGCTGGGCAC |
| | | | TCAGACGTATATTTGCAATGTGAACCACAAACCTTCAAATACA |
| | | | AAAGTGGATAAACGCGTAGAACCGAAATCGTGTGATAAAACT |
| | | | CACACATGCCCGCCATGCCCGGCACCTGAACTGCTTGGTGGTC |
| | | | CCAGCGTGTTCCTGTTCCCGCCGAAGCCTAAAGATACTCTAAT |
| | | | GATCAGCCGTACGCCAGAGGTGACATGTGTCGTGGTTGACGTG |
| | | | TCCCACGAAGATCCCGAAGTTAAGTTCAATTGGTATGTTGATG |
| | | | GTGTAGAGGTACACAATGCTAAGACTAAACCTCGCGAGGAGC |
| | | | AGTACGCCTCGACCTATCGTGTCGTGAGCGTTCTGACCGTCCT |
| | | | TCACCAAGATTGGCTTAACGGCAAAGAATATAAGTGCAAGGT |
| | | | AAGCAATAAAGCACTTCCGGCCCCAATCGAGAAAACCATTTCC |
| | | | AAGGCCAAAGGTCAACCAAGAGAACCCCAGGTGTATACTCTT |
| | | | CCGCCTTCTCGTGAGGAAATGACTAAAAATCAAGTATCCCTTA |
| | | | CGTGTCTGGTTAAAGGTTTTTATCCTAGCGATATTGCTGTTGAA |
| | | | TGGGAATCGAACGGTCAGCCGGAGAATAATTATAAAACAACG |
| | | | CCACCCGTCCTGGATAGCGACGGCTCATTTTTTCTGTATAGCA |
| | | | AACTGACTGTAGATAAATCACGGTGGCAGCAGGGCAATGTAT |
| | | | TCAGTTGCTCCGTTATGCATGAAGCGTTACATAATCACTACAC |
| | | | GCAGAAATCTCTTAGTCTTTCACCCGGT |
| 1350 | ATX-P-3362 | HC IgG1 Fc nt | GAAGTGCAGCTGGTGGAGTCTGGGGGAGGCTTGGTACAGCCT |
| | | | GGCAGGTCCCTGAGACTCTCCTGTGCAGCCTCTGGATTCACCT |
| | | | TTGATGATTATGCCATGCACTGGGTCCGGCAAGCTCCAGGGAA |
| | | | GGGCCTGGAGTGGGTCTCAGGTATTAGTTGGAATAGTGGTAGC |
| | | | ATAGCCTATGCGGACTCTGTGAAGGGCCGATTCACCATCTCCA |
| | | | GAGACAACGCCAAGAACTCCCTGTATCTGCAAATGAACAGTCT |
| | | | GAGAGTTGAGGACACGGCCTTTTTTTACTGTGCAAGAGTCGCA |
| | | | GCAGCAGCTGGTATCCCTTTCTTTGACTACTGGGGCCAGGGAA |
| | | | CCCTGGTCACCGTCTCCTCAGCTAGCACTAAAGGGCCTTCTGT |
| | | | ATTTCCCTTGGCCCCGTCCAGCAAATCGACCTCGGGAGGGACA |
| | | | GCCGCCCTGGGTTGCCTTGTGAAAGATTATTTCCCTGAGCCAG |
| | | | TTACCGTAAGTTGGAACAGTGGGGCGCTGACAAGTGGTGTGC |
| | | | ACACGTTTCCTGCCGTCCTGCAATCATCGGGCTTGTATAGCCTC |
| | | | AGCTCTGTGGTCACTGTCCCAAGTTCATCGCTGGGCACTCAGA |
| | | | CGTATATTTGCAATGTGAACCACAAACCTTCAAATACAAAAGT |
| | | | GGATAAACGCGTAGAACCGAAATCGTGTGATAAAACTCACAC |
| | | | ATGCCCGCCATGCCCGGCACCTGAACTGCTTGGTGGTCCCAGC |
| | | | GTGTTCCTGTTCCCGCCGAAGCCTAAAGATACTCTAATGATCA |
| | | | GCCGTACGCCAGAGGTGACATGTGTCGTGGTTGACGTGTCCCA |
| | | | CGAAGATCCCGAAGTTAAGTTCAATTGGTATGTTGATGGTGTA |
| | | | GAGGTACACAATGCTAAGACTAAACCTCGCGAGGAGCAGTAC |
| | | | GCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1351 | ATX-P-3363 | HC IgG1 Fc nt | CAGGTGCAGCTGCAGGAGTCGGGCCCAGGACTGGTGAAGCCT |
| | | | TCTGGGACCCTGACCCTCACCTGCGCTGTCTCTGGTGGCTCCAT |
| | | | CAACAGGGATAACTGGTGGAGTTGGGTCCGCCAGCCCCCGGG |
| | | | GAAGGGGCTGGAGTGGATTGGACAAATCTATCAGAGTGGGAG |
| | | | CACCAACTACAACCCGTCCCTCAAGAGTCGGGTCACCATATCA |
| | | | GTCGACAAGTCCAAGAACCAGTTCTCCCTGAAGCTGAGTTCTG |
| | | | TGACCGCCGCGGACACGGCCGTATATTACTGTGCGAGAGTATA |
| | | | TGACTGGAACTACGGGGGTCACTTTGACTACTGGGGCCAGGG |
| | | | AACCCTGGTCACCGTCTCCTCAGCTAGCACTAAAGGGCCTTCT |
| | | | GTATTTCCCTTGGCCCCGTCCAGCAAATCGACCTCGGGAGGGA |
| | | | CAGCCGCCCTGGGTTGCCTTGTGAAAGATTATTTCCCTGAGCC |
| | | | AGTTACCGTAAGTTGGAACAGTGGGGCGCTGACAAGTGGTGT |
| | | | GCACACGTTTCCTGCCGTCCTGCAATCATCGGGCTTGTATAGC |
| | | | CTCAGCTCTGTGGTCACTGTCCCAAGTTCATCGCTGGGCACTC |
| | | | AGACGTATATTTGCAATGTGAACCACAAACCTTCAAATACAAA |
| | | | AGTGGATAAACGCGTAGAACCGAAATCGTGTGATAAAACTCA |
| | | | CACATGCCCGCCATGCCCGGCACCTGAACTGCTTGGTGGTCCC |
| | | | AGCGTGTTCCTGTTCCCGCCGAAGCCTAAAGATACTCTAATGA |
| | | | TCAGCCGTACGCCAGAGGTGACATGTGTCGTGGTTGACGTGTC |
| | | | CCACGAAGATCCCGAAGTTAAGTTCAATTGGTATGTTGATGGT |
| | | | GTAGAGGTACACAATGCTAAGACTAAACCTCGCGAGGAGCAG |
| | | | TACGCCTCGACCTATCGTGTCGTGAGCGTTCTGACCGTCCTTCA |
| | | | CCAAGATTGGCTTAACGGCAAAGAATATAAGTGCAAGGTAAG |
| | | | CAATAAAGCACTTCCGGCCCCAATCGAGAAAACCATTTCCAAG |
| | | | GCCAAAGGTCAACCAAGAGAACCCCAGGTGTATACTCTTCCGC |
| | | | CTTCTCGTGAGGAAATGACTAAAAATCAAGTATCCCTTACGTG |
| | | | TCTGGTTAAAGGTTTTTATCCTAGCGATATTGCTGTTGAATGGG |
| | | | AATCGAACGGTCAGCCGGAGAATAATTATAAAACAACGCCAC |
| | | | CCGTCCTGGATAGCGACGGCTCATTTTTTCTGTATAGCAAACT |
| | | | GACTGTAGATAAATCACGGTGGCAGCAGGGCAATGTATTCAG |
| | | | TTGCTCCGTTATGCATGAAGCGTTACATAATCACTACACGCAG |
| | | | AAATCTCTTAGTCTTTCACCCGGT |
| 1352 | ATX-P-3365 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGACTCCAT |
| | | | CAGCAGTAATAACTGGTGGAGTTGGGTCCGCCAGCCCCCAGG |
| | | | GAAGGGGCTGGAGTGGATTGGACAAATCTATCATAGTGGCCA |
| | | | CACCAACTACAACCCGTCCCTCAAGAGTCGAGTCACCATATCG |
| | | | GTTGACATGTCCAAGAACCAGTTCTCCCTGAAGCTGACCTCTG |
| | | | TGACCGCCGCGGACACGGCCGTGTATTACTGTGCGAGGGGGG |
| | | | CTACAGAAATTCTGGGGTACTTCGATCTCTGGGGCCGTGGCAC |
| | | | CCTGGTCACTGTCTCCTCAGCTAGCACTAAAGGGCCTTCTGTA |
| | | | TTTCCCTTGGCCCCGTCCAGCAAATCGACCTCGGGAGGGACAG |
| | | | CCGCCCTGGGTTGCCTTGTGAAAGATTATTTCCCTGAGCCAGT |
| | | | TACCGTAAGTTGGAACAGTGGGGCGCTGACAAGTGGTGTGCA |
| | | | CACGTTTCCTGCCGTCCTGCAATCATCGGGCTTGTATAGCCTCA |
| | | | GCTCTGTGGTCACTGTCCCAAGTTCATCGCTGGGCACTCAGAC |
| | | | GTATATTTGCAATGTGAACCACAAACCTTCAAATACAAAAGTG |
| | | | GATAAACGCGTAGAACCGAAATCGTGTGATAAAACTCACACA |
| | | | TGCCCGCCATGCCCGGCACCTGAACTGCTTGGTGGTCCCAGCG |
| | | | TGTTCCTGTTCCCGCCGAAGCCTAAAGATACTCTAATGATCAG |
| | | | CCGTACGCCAGAGGTGACATGTGTCGTGGTTGACGTGTCCCAC |
| | | | GAAGATCCCGAAGTTAAGTTCAATTGGTATGTTGATGGTGTAG |
| | | | AGGTACACAATGCTAAGACTAAACCTCGCGAGGAGCAGTACG |
| | | | CCTCGACCTATCGTGTCGTGAGCGTTCTGACCGTCCTTCACCA |
| | | | AGATTGGCTTAACGGCAAAGAATATAAGTGCAAGGTAAGCAA |
| | | | TAAAGCACTTCCGGCCCCAATCGAGAAAACCATTTCCAAGGCC |
| | | | AAAGGTCAACCAAGAGAACCCCAGGTGTATACTCTTCCGCCTT |
| | | | CTCGTGAGGAAATGACTAAAAATCAAGTATCCCTTACGTGTCT |
| | | | GGTTAAAGGTTTTTATCCTAGCGATATTGCTGTTGAATGGGAA |
| | | | TCGAACGGTCAGCCGGAGAATAATTATAAAACAACGCCACCC |
| | | | GTCCTGGATAGCGACGGCTCATTTTTTCTGTATAGCAAACTGA |
| | | | CTGTAGATAAATCACGGTGGCAGCAGGGCAATGTATTCAGTTG |
| | | | CTCCGTTATGCATGAAGCGTTACATAATCACTACACGCAGAAA |
| | | | TCTCTTAGTCTTTCACCCGGT |
| 1353 | ATX-P-3367 | HC IgG1 Fc nt | CAGGTGCAGCTGCAGGAGTCGGGCCCAGGACTGGTGAAGCCT |
| | | | TCGGAGACCCTGTCCCTCACCTGCACTGTCTCTGGTGGCTCCGT |
| | | | CAGCAGTGGTAGTTACTACTGGAGCTGGATCCGGCAGCCCCCA |
| | | | GGGAAGGGACTGGAGTGGATTGGATATATCTATAAAAGTGGG |
| | | | TTCACCAATTACAACCCCTCCCTCAAGAGTCGAGTCACCATAT |
| | | | CAGTAGACACGTCCAAGAACCAGTTCTCCCTGAAGCTGAGCTC |
| | | | TGTGACCGCTGCGGACACGGCCGTGTATTACTGTGCGATAGGG |
| | | | GGACGTGGATACAGCTATGGTCCTTTTGACTACTGGGGCCAGG |
| | | | GAACCCTGGTCACCGTCTCCTCAGCTAGCACTAAAGGGCCTTC |
| | | | TGTATTTCCCTTGGCCCCGTCCAGCAAATCGACCTCGGGAGGG |
| | | | ACAGCCGCCCTGGGTTGCCTTGTGAAAGATTATTTCCCTGAGC |
| | | | CAGTTACCGTAAGTTGGAACAGTGGGGCGCTGACAAGTGGTG |
| | | | TGCACACGTTTCCTGCCGTCCTGCAATCATCGGGCTTGTATAG |
| | | | CCTCAGCTCTGTGGTCACTGTCCCAAGTTCATCGCTGGGCACT |
| | | | CAGACGTATATTTGCAATGTGAACCACAAACCTTCAAATACAA |
| | | | AAGTGGATAAACGCGTAGAACCGAAATCGTGTGATAAAACTC |
| | | | ACACATGCCCGCCATGCCCGGCACCTGAACTGCTTGGTGGTCC |
| | | | CAGCGTGTTCCTGTTCCCGCCGAAGCCTAAAGATACTCTAATG |
| | | | ATCAGCCGTACGCCAGAGGTGACATGTGTCGTGGTTGACGTGT |
| | | | CCCACGAAGATCCCGAAGTTAAGTTCAATTGGTATGTTGATGG |
| | | | TGTAGAGGTACACAATGCTAAGACTAAACCTCGCGAGGAGCA |
| | | | GTACGCCTCGACCTATCGTGTCGTGAGCGTTCTGACCGTCCTTC |
| | | | ACCAAGATTGGCTTAACGGCAAAGAATATAAGTGCAAGGTAA |
| | | | GCAATAAAGCACTTCCGGCCCCAATCGAGAAAACCATTTCCAA |
| | | | GGCCAAAGGTCAACCAAGAGAACCCCAGGTGTATACTCTTCC |
| | | | GCCTTCTCGTGAGGAAATGACTAAAAATCAAGTATCCCTTACG |
| | | | TGTCTGGTTAAAGGTTTTTATCCTAGCGATATTGCTGTTGAATG |
| | | | GGAATCGAACGGTCAGCCGGAGAATAATTATAAAACAACGCC |
| | | | ACCCGTCCTGGATAGCGACGGCTCATTTTTTCTGTATAGCAAA |
| | | | CTGACTGTAGATAAATCACGGTGGCAGCAGGGCAATGTATTCA |
| | | | GTTGCTCCGTTATGCATGAAGCGTTACATAATCACTACACGCA |
| | | | GAAATCTCTTAGTCTTTCACCCGGT |
| 1354 | ATX-P-3369 | HC IgG1 Fc nt | CAGGTGCAGCTGGTGCAGTCTGGGGCTGAGGTGAAGAAGCCT |
| | | | GGGGCCTCAGTGAAGGTCTCCTGCAAGGCTTCTGGATACACCT |
| | | | TCACCGGCTACTATTTACACTGGGTGCGACAGGCCCCTGGACA |
| | | | AGGACTTGAGTGGATGGGATGGATCAACCCTAACAGTGGTGA |
| | | | CACAAACTATGTACAGAACTTTCAGGGCAGGGTCACCATGACC |
| | | | AGGGACACGTCCATCAGCACAGCCTACATGGAGCTGAGCAGG |
| | | | CTGAGATCTGACGACACGGCCGTGTATTACTGTGCGAGATCCT |
| | | | ACGTGGATACCAGCTCGTCGGCTTTTGATATCTGGGGACAAGG |
| | | | GACAATGGTCACCGTCTCTTCAGCTAGCACTAAAGGGCCTTCT |
| | | | GTATTTCCCTTGGCCCCGTCCAGCAAATCGACCTCGGGAGGGA |
| | | | CAGCCGCCCTGGGTTGCCTTGTGAAAGATTATTTCCCTGAGCC |
| | | | AGTTACCGTAAGTTGGAACAGTGGGGCGCTGACAAGTGGTGT |
| | | | GCACACGTTTCCTGCCGTCCTGCAATCATCGGGCTTGTATAGC |
| | | | CTCAGCTCTGTGGTCACTGTCCCAAGTTCATCGCTGGGCACTC |
| | | | AGACGTATATTTGCAATGTGAACCACAAACCTTCAAATACAAA |
| | | | AGTGGATAAACGCGTAGAACCGAAATCGTGTGATAAAACTCA |
| | | | CACATGCCCGCCATGCCCGGCACCTGAACTGCTTGGTGGTCCC |
| | | | AGCGTGTTCCTGTTCCCGCCGAAGCCTAAAGATACTCTAATGA |
| | | | TCAGCCGTACGCCAGAGGTGACATGTGTCGTGGTTGACGTGTC |
| | | | CCACGAAGATCCCGAAGTTAAGTTCAATTGGTATGTTGATGGT |
| | | | GTAGAGGTACACAATGCTAAGACTAAACCTCGCGAGGAGCAG |
| | | | TACGCCTCGACCTATCGTGTCGTGAGCGTTCTGACCGTCCTTCA |
| | | | CCAAGATTGGCTTAACGGCAAAGAATATAAGTGCAAGGTAAG |
| | | | CAATAAAGCACTTCCGGCCCCAATCGAGAAAACCATTTCCAAG |
| | | | GCCAAAGGTCAACCAAGAGAACCCCAGGTGTATACTCTTCCGC |
| | | | CTTCTCGTGAGGAAATGACTAAAAATCAAGTATCCCTTACGTG |
| | | | TCTGGTTAAAGGTTTTTATCCTAGCGATATTGCTGTTGAATGGG |
| | | | AATCGAACGGTCAGCCGGAGAATAATTATAAAACAACGCCAC |
| | | | CCGTCCTGGATAGCGACGGCTCATTTTTTCTGTATAGCAAACT |
| | | | GACTGTAGATAAATCACGGTGGCAGCAGGGCAATGTATTCAG |
| | | | TTGCTCCGTTATGCATGAAGCGTTACATAATCACTACACGCAG |
| | | | AAATCTCTTAGTCTTTCACCCGGT |
| 1355 | ATX-P-3370 | HC IgG1 Fc nt | CAGGTGCAGCTGCAGGAGTCGGGCCCAGGACTGGTGAAGCCT |
| | | | TCGGAGACCCTGTCCCTCACCTGCACTGTCTCTGGTGGCTCCAT |
| | | | CAGTAGTTACTACTGGAGCTGGATCCGGCAGCCCCCCGGGAA |
| | | | GGGACTGGAGTGGATTGGGTATGTCTATTACAGTGGGTACACC |
| | | | AAGTACAACCCCTCCCTCAAGAGTCGAGTCACCATATCAGTAG |
| | | | ACACGCCCAAGACCCAGTTCTCCCTGAAGATGAGCTCTGTGAC |
| | | | CGCTGCGGACACGGCCGTGTATTACTGTGCGAGCGTCGAATCA |
| | | | AAGTGGAACTACGGGCCTTTTGATATCTGGGGACAAGGGACA |
| | | | ATGGTCACCGTCTCTTCAGCTAGCACTAAAGGGCCTTCTGTAT |
| | | | TTCCCTTGGCCCCGTCCAGCAAATCGACCTCGGGAGGGACAGC |
| | | | CGCCCTGGGTTGCCTTGTGAAAGATTATTTCCCTGAGCCAGTT |
| | | | ACCGTAAGTTGGAACAGTGGGGCGCTGACAAGTGGTGTGCAC |
| | | | ACGTTTCCTGCCGTCCTGCAATCATCGGGCTTGTATAGCCTCA |
| | | | GCTCTGTGGTCACTGTCCCAAGTTCATCGCTGGGCACTCAGAC |
| | | | GTATATTTGCAATGTGAACCACAAACCTTCAAATACAAAAGTG |
| | | | GATAAACGCGTAGAACCGAAATCGTGTGATAAAACTCACACA |
| | | | TGCCCGCCATGCCCGGCACCTGAACTGCTTGGTGGTCCCAGCG |
| | | | TGTTCCTGTTCCCGCCGAAGCCTAAAGATACTCTAATGATCAG |
| | | | CCGTACGCCAGAGGTGACATGTGTCGTGGTTGACGTGTCCCAC |
| | | | GAAGATCCCGAAGTTAAGTTCAATTGGTATGTTGATGGTGTAG |
| | | | AGGTACACAATGCTAAGACTAAACCTCGCGAGGAGCAGTACG |
| | | | CCTCGACCTATCGTGTCGTGAGCGTTCTGACCGTCCTTCACCA |
| | | | AGATTGGCTTAACGGCAAAGAATATAAGTGCAAGGTAAGCAA |
| | | | TAAAGCACTTCCGGCCCCAATCGAGAAAACCATTTCCAAGGCC |
| | | | AAAGGTCAACCAAGAGAACCCCAGGTGTATACTCTTCCGCCTT |
| | | | CTCGTGAGGAAATGACTAAAAATCAAGTATCCCTTACGTGTCT |
| | | | GGTTAAAGGTTTTTATCCTAGCGATATTGCTGTTGAATGGGAA |
| | | | TCGAACGGTCAGCCGGAGAATAATTATAAAACAACGCCACCC |
| | | | GTCCTGGATAGCGACGGCTCATTTTTTCTGTATAGCAAACTGA |
| | | | CTGTAGATAAATCACGGTGGCAGCAGGGCAATGTATTCAGTTG |
| | | | CTCCGTTATGCATGAAGCGTTACATAATCACTACACGCAGAAA |
| | | | TCTCTTAGTCTTTCACCCGGT |
| 1356 | ATX-P-3371 | HC IgG1 Fc nt | CAGGTGCAGCTGCAGGAGTCGGGCCCAGGACTGGTGAAGCCT |
| | | | TCGGAGACCCTGTCCCTCACCTGCACTGTCTCTGGTGACTCCAT |
| | | | CAGTAGTTACTACTGGAGCTGGATCCGGCAGCCCCCAGGGAA |
| | | | GGGACTGGAGTGGATTGGATATATCTATTACAGTGGGTTCACC |
| | | | AAGTACAACCCCTCCCTCAACAGTCGAGTCACCATATCAGTAG |
| | | | ACACGTCCAAGAGCCAGTTCTCCCTGAAGCTGACCTCTGTGAC |
| | | | CGCTGCGGACACGGCCGTGTATTACTGTGCGATGAGAGAGCC |
| | | | GAGAGGCCGGTATAACTGGAACTACGGGCCTTTTGATATCTGG |
| | | | GGACAAGGGACAATGGTCACCGTCTCTTCAGCTAGCACTAAA |
| | | | GGGCCTTCTGTATTTCCCTTGGCCCCGTCCAGCAAATCGACCTC |
| | | | GGGAGGGACAGCCGCCCTGGGTTGCCTTGTGAAAGATTATTTC |
| | | | CCTGAGCCAGTTACCGTAAGTTGGAACAGTGGGGCGCTGACA |
| | | | AGTGGTGTGCACACGTTTCCTGCCGTCCTGCAATCATCGGGCT |
| | | | TGTATAGCCTCAGCTCTGTGGTCACTGTCCCAAGTTCATCGCTG |
| | | | GGCACTCAGACGTATATTTGCAATGTGAACCACAAACCTTCAA |
| | | | ATACAAAAGTGGATAAACGCGTAGAACCGAAATCGTGTGATA |
| | | | AAACTCACACATGCCCGCCATGCCCGGCACCTGAACTGCTTGG |
| | | | TGGTCCCAGCGTGTTCCTGTTCCCGCCGAAGCCTAAAGATACT |
| | | | CTAATGATCAGCCGTACGCCAGAGGTGACATGTGTCGTGGTTG |
| | | | ACGTGTCCCACGAAGATCCCGAAGTTAAGTTCAATTGGTATGT |
| | | | TGATGGTGTAGAGGTACACAATGCTAAGACTAAACCTCGCGA |
| | | | GGAGCAGTACGCCTCGACCTATCGTGTCGTGAGCGTTCTGACC |
| | | | GTCCTTCACCAAGATTGGCTTAACGGCAAAGAATATAAGTGCA |
| | | | AGGTAAGCAATAAAGCACTTCCGGCCCCAATCGAGAAAACCA |
| | | | TTTCCAAGGCCAAAGGTCAACCAAGAGAACCCCAGGTGTATA |
| | | | CTCTTCCGCCTTCTCGTGAGGAAATGACTAAAAATCAAGTATC |
| | | | CCTTACGTGTCTGGTTAAAGGTTTTTATCCTAGCGATATTGCTG |
| | | | TTGAATGGGAATCGAACGGTCAGCCGGAGAATAATTATAAAA |
| | | | CAACGCCACCCGTCCTGGATAGCGACGGCTCATTTTTTCTGTA |
| | | | TAGCAAACTGACTGTAGATAAATCACGGTGGCAGCAGGGCAA |
| | | | TGTATTCAGTTGCTCCGTTATGCATGAAGCGTTACATAATCACT |
| | | | ACACGCAGAAATCTCTTAGTCTTTCACCCGGT |
| 1357 | ATX-P-3373 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGTAATGATAACTGGTGGAGTTGGGTCCGCCAGCCCCCCGGG |
| | | | AAGGGACTGGAGTGGATTGGGAAAATCTATCATAGTGAGTTC |
| | | | ACCAACTACAACCCGTCCCTCAAGAGTCGAGTCACCATATCAG |
| | | | TAGACAAGTCCAAGAACCAGTTCTCCCTGGAGCTGAACTCTGT |
| | | | GACCGCCGCGGACACGGCCGTGTATTACTGTGCGAGAGAGTA |
| | | | TCATAATTTCATGGACGTCTGGTGCAAAGGGACCACGGTCACC |
| | | | GTCTCCTCAGCTAGCACTAAAGGGCCTTCTGTATTTCCCTTGGC |
| | | | CCCGTCCAGCAAATCGACCTCGGGAGGGACAGCCGCCCTGGG |
| | | | TTGCCTTGTGAAAGATTATTTCCCTGAGCCAGTTACCGTAAGTT |
| | | | GGAACAGTGGGGCGCTGACAAGTGGTGTGCACACGTTTCCTGC |
| | | | CGTCCTGCAATCATCGGGCTTGTATAGCCTCAGCTCTGTGGTC |
| | | | ACTGTCCCAAGTTCATCGCTGGGCACTCAGACGTATATTTGCA |
| | | | ATGTGAACCACAAACCTTCAAATACAAAAGTGGATAAACGCG |
| | | | TAGAACCGAAATCGTGTGATAAAACTCACACATGCCCGCCATG |
| | | | CCCGGCACCTGAACTGCTTGGTGGTCCCAGCGTGTTCCTGTTC |
| | | | CCGCCGAAGCCTAAAGATACTCTAATGATCAGCCGTACGCCAG |
| | | | AGGTGACATGTGTCGTGGTTGACGTGTCCCACGAAGATCCCGA |
| | | | AGTTAAGTTCAATTGGTATGTTGATGGTGTAGAGGTACACAAT |
| | | | GCTAAGACTAAACCTCGCGAGGAGCAGTACGCCTCGACCTATC |
| | | | GTGTCGTGAGCGTTCTGACCGTCCTTCACCAAGATTGGCTTAA |
| | | | CGGCAAAGAATATAAGTGCAAGGTAAGCAATAAAGCACTTCC |
| | | | GGCCCCAATCGAGAAAACCATTTCCAAGGCCAAAGGTCAACC |
| | | | AAGAGAACCCCAGGTGTATACTCTTCCGCCTTCTCGTGAGGAA |
| | | | ATGACTAAAAATCAAGTATCCCTTACGTGTCTGGTTAAAGGTT |
| | | | TTTATCCTAGCGATATTGCTGTTGAATGGGAATCGAACGGTCA |
| | | | GCCGGAGAATAATTATAAAACAACGCCACCCGTCCTGGATAG |
| | | | CGACGGCTCATTTTTTCTGTATAGCAAACTGACTGTAGATAAA |
| | | | TCACGGTGGCAGCAGGGCAATGTATTCAGTTGCTCCGTTATGC |
| | | | ATGAAGCGTTACATAATCACTACACGCAGAAATCTCTTAGTCT |
| | | | TTCACCCGGT |
| 1358 | ATX-P-3375 | HC IgG1 Fc nt | CAGGTGCAGCTGCAGGAGTCGGGCCCAGGACTGGTGAAGCCT |
| | | | TCGGAGACCCTGTCCCTCACCTGCACTGTCTCTGGTGACTCCGT |
| | | | CAGCAGTGGTAAATACTTCTGGAGCTGGATCCGGCAGTCCCCA |
| | | | GGGAAGGGACTGGAGTGGATTGGGTATATCTATTACAGTGGG |
| | | | AGCACCAACTACAACCCCTCCCTCAAGAATCGAGTCACCATAT |
| | | | CGGTAGACACGTCCAAGAAGGAGTTGTCTCTGAAGCTGAGTTC |
| | | | TGTGACCGCTGCGGACACGGCCGTTTATTACTGTGCGAGAGGA |
| | | | GGGCATAACTGGAACTACGCTGCTTTTGATATCTGGGGACAAG |
| | | | GGACAATGGTCACCGTCTCTTCAGCTAGCACTAAAGGGCCTTC |
| | | | TGTATTTCCCTTGGCCCCGTCCAGCAAATCGACCTCGGGAGGG |
| | | | ACAGCCGCCCTGGGTTGCCTTGTGAAAGATTATTTCCCTGAGC |
| | | | CAGTTACCGTAAGTTGGAACAGTGGGGCGCTGACAAGTGGTG |
| | | | TGCACACGTTTCCTGCCGTCCTGCAATCATCGGGCTTGTATAG |
| | | | CCTCAGCTCTGTGGTCACTGTCCCAAGTTCATCGCTGGGCACT |
| | | | CAGACGTATATTTGCAATGTGAACCACAAACCTTCAAATACAA |
| | | | AAGTGGATAAACGCGTAGAACCGAAATCGTGTGATAAAACTC |
| | | | ACACATGCCCGCCATGCCCGGCACCTGAACTGCTTGGTGGTCC |
| | | | CAGCGTGTTCCTGTTCCCGCCGAAGCCTAAAGATACTCTAATG |
| | | | ATCAGCCGTACGCCAGAGGTGACATGTGTCGTGGTTGACGTGT |
| | | | CCCACGAAGATCCCGAAGTTAAGTTCAATTGGTATGTTGATGG |
| | | | TGTAGAGGTACACAATGCTAAGACTAAACCTCGCGAGGAGCA |
| | | | GTACGCCTCGACCTATCGTGTCGTGAGCGTTCTGACCGTCCTTC |
| | | | ACCAAGATTGGCTTAACGGCAAAGAATATAAGTGCAAGGTAA |
| | | | GCAATAAAGCACTTCCGGCCCCAATCGAGAAAACCATTTCCAA |
| | | | GGCCAAAGGTCAACCAAGAGAACCCCAGGTGTATACTCTTCC |
| | | | GCCTTCTCGTGAGGAAATGACTAAAAATCAAGTATCCCTTACG |
| | | | TGTCTGGTTAAAGGTTTTTATCCTAGCGATATTGCTGTTGAATG |
| | | | GGAATCGAACGGTCAGCCGGAGAATAATTATAAAACAACGCC |
| | | | ACCCGTCCTGGATAGCGACGGCTCATTTTTTCTGTATAGCAAA |
| | | | CTGACTGTAGATAAATCACGGTGGCAGCAGGGCAATGTATTCA |
| | | | GTTGCTCCGTTATGCATGAAGCGTTACATAATCACTACACGCA |
| | | | GAAATCTCTTAGTCTTTCACCCGGT |
| 1359 | ATX-P-3378 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGTAATGATAACTGGTGGAGTTGGGTCCGCCAGTCCCCCGGG |
| | | | AAGGGGTTGGAGTGGATTGGGAAAATCTATCATAGTGAGTTC |
| | | | ACCAACTACAACCCGTCCCTCAAGAGTCGAGTCACCATATCAG |
| | | | TAGACAAGTCCAAGAACCAGTTCTCCCTGGAGCTGAGCTCTGT |
| | | | GGCCGCCGCGGACACGGCCGTGTATTACTGTGCGAGAGAGTA |
| | | | TGAGAACTTCATGGACGTCTGGGGCAAGGGGACCACGGTCAC |
| | | | CGTCTCCTCAGCTAGCACTAAAGGGCCTTCTGTATTTCCCTTGG |
| | | | CCCCGTCCAGCAAATCGACCTCGGGAGGGACAGCCGCCCTGG |
| | | | GTTGCCTTGTGAAAGATTATTTCCCTGAGCCAGTTACCGTAAG |
| | | | TTGGAACAGTGGGGCGCTGACAAGTGGTGTGCACACGTTTCCT |
| | | | GCCGTCCTGCAATCATCGGGCTTGTATAGCCTCAGCTCTGTGG |
| | | | TCACTGTCCCAAGTTCATCGCTGGGCACTCAGACGTATATTTG |
| | | | CAATGTGAACCACAAACCTTCAAATACAAAAGTGGATAAACG |
| | | | CGTAGAACCGAAATCGTGTGATAAAACTCACACATGCCCGCC |
| | | | ATGCCCGGCACCTGAACTGCTTGGTGGTCCCAGCGTGTTCCTG |
| | | | TTCCCGCCGAAGCCTAAAGATACTCTAATGATCAGCCGTACGC |
| | | | CAGAGGTGACATGTGTCGTGGTTGACGTGTCCCACGAAGATCC |
| | | | CGAAGTTAAGTTCAATTGGTATGTTGATGGTGTAGAGGTACAC |
| | | | AATGCTAAGACTAAACCTCGCGAGGAGCAGTACGCCTCGACC |
| | | | TATCGTGTCGTGAGCGTTCTGACCGTCCTTCACCAAGATTGGC |
| | | | TTAACGGCAAAGAATATAAGTGCAAGGTAAGCAATAAAGCAC |
| | | | TTCCGGCCCCAATCGAGAAAACCATTTCCAAGGCCAAAGGTCA |
| | | | ACCAAGAGAACCCCAGGTGTATACTCTTCCGCCTTCTCGTGAG |
| | | | GAAATGACTAAAAATCAAGTATCCCTTACGTGTCTGGTTAAAG |
| | | | GTTTTTATCCTAGCGATATTGCTGTTGAATGGGAATCGAACGG |
| | | | TCAGCCGGAGAATAATTATAAAACAACGCCACCCGTCCTGGAT |
| | | | AGCGACGGCTCATTTTTTCTGTATAGCAAACTGACTGTAGATA |
| | | | AATCACGGTGGCAGCAGGGCAATGTATTCAGTTGCTCCGTTAT |
| | | | GCATGAAGCGTTACATAATCACTACACGCAGAAATCTCTTAGT |
| | | | CTTTCACCCGGT |
| 1360 | ATX-P-3382 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGACTCCAT |
| | | | CAGCAGTAATAGCTGGTGGAATTGGGTCCGCCAGCCCCCAGG |
| | | | GAAGGGGCTGGAGTGGATTGGGCAAATCTTTCATAGTGGACA |
| | | | CACCAACTACAACCCGTCCCTCAAGAGTCGAGTCACCATGTCT |
| | | | ATAGACAAGTCCAAGAACCAGTTCTCCCTGAAACTGACCTCTG |
| | | | TGACCGCCGCGGACACGGCCCTGTATTACTGTGCGAGAGGAG |
| | | | GGGAGTATGAAAACTCGGCGAACTGGTTCGACCCCTGGGGCC |
| | | | AAGGAACCCTGGTCACCGTCTCTTCAGCTAGCACTAAAGGGCC |
| | | | TTCTGTATTTCCCTTGGCCCCGTCCAGCAAATCGACCTCGGGA |
| | | | GGGACAGCCGCCCTGGGTTGCCTTGTGAAAGATTATTTCCCTG |
| | | | AGCCAGTTACCGTAAGTTGGAACAGTGGGGCGCTGACAAGTG |
| | | | GTGTGCACACGTTTCCTGCCGTCCTGCAATCATCGGGCTTGTAT |
| | | | AGCCTCAGCTCTGTGGTCACTGTCCCAAGTTCATCGCTGGGCA |
| | | | CTCAGACGTATATTTGCAATGTGAACCACAAACCTTCAAATAC |
| | | | AAAAGTGGATAAACGCGTAGAACCGAAATCGTGTGATAAAAC |
| | | | TCACACATGCCCGCCATGCCCGGCACCTGAACTGCTTGGTGGT |
| | | | CCCAGCGTGTTCCTGTTCCCGCCGAAGCCTAAAGATACTCTAA |
| | | | TGATCAGCCGTACGCCAGAGGTGACATGTGTCGTGGTTGACGT |
| | | | GTCCCACGAAGATCCCGAAGTTAAGTTCAATTGGTATGTTGAT |
| | | | GGTGTAGAGGTACACAATGCTAAGACTAAACCTCGCGAGGAG |
| | | | CAGTACGCCTCGACCTATCGTGTCGTGAGCGTTCTGACCGTCC |
| | | | TTCACCAAGATTGGCTTAACGGCAAAGAATATAAGTGCAAGG |
| | | | TAAGCAATAAAGCACTTCCGGCCCCAATCGAGAAAACCATTTC |
| | | | CAAGGCCAAAGGTCAACCAAGAGAACCCCAGGTGTATACTCT |
| | | | TCCGCCTTCTCGTGAGGAAATGACTAAAAATCAAGTATCCCTT |
| | | | ACGTGTCTGGTTAAAGGTTTTTATCCTAGCGATATTGCTGTTGA |
| | | | ATGGGAATCGAACGGTCAGCCGGAGAATAATTATAAAACAAC |
| | | | GCCACCCGTCCTGGATAGCGACGGCTCATTTTTTCTGTATAGC |
| | | | AAACTGACTGTAGATAAATCACGGTGGCAGCAGGGCAATGTA |
| | | | TTCAGTTGCTCCGTTATGCATGAAGCGTTACATAATCACTACA |
| | | | CGCAGAAATCTCTTAGTCTTTCACCCGGT |
| 1361 | ATX-P-3383 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGTCTCCAT |
| | | | CAACAAGGATAACTGGTGGACTTGGGTCCGCCAGCCCCCAGG |
| | | | GAAGGGGCTGGAGTGGATTGGGAAAATCTATCATAGTGGGAG |
| | | | CACCAACTACAACCCGTCCCTCAAGAGTCGAGTCACCATATCA |
| | | | GTAGACAACTCCAAGAACCAATTCTCCCTGAAGCTGAACTCTG |
| | | | TGACCGCCGCGGACACGGCCGTGTATTACTGTACGAGAGGGG |
| | | | GGGATATAGCTATGGAGGGGGACTGGTTCGCCCCCTGGGGCC |
| | | | AGGGAACCCTGGTCACCGTCTCCTCAGCTAGCACTAAAGGGCC |
| | | | TTCTGTATTTCCCTTGGCCCCGTCCAGCAAATCGACCTCGGGA |
| | | | GGGACAGCCGCCCTGGGTTGCCTTGTGAAAGATTATTTCCCTG |
| | | | AGCCAGTTACCGTAAGTTGGAACAGTGGGGCGCTGACAAGTG |
| | | | GTGTGCACACGTTTCCTGCCGTCCTGCAATCATCGGGCTTGTAT |
| | | | AGCCTCAGCTCTGTGGTCACTGTCCCAAGTTCATCGCTGGGCA |
| | | | CTCAGACGTATATTTGCAATGTGAACCACAAACCTTCAAATAC |
| | | | AAAAGTGGATAAACGCGTAGAACCGAAATCGTGTGATAAAAC |
| | | | TCACACATGCCCGCCATGCCCGGCACCTGAACTGCTTGGTGGT |
| | | | CCCAGCGTGTTCCTGTTCCCGCCGAAGCCTAAAGATACTCTAA |
| | | | TGATCAGCCGTACGCCAGAGGTGACATGTGTCGTGGTTGACGT |
| | | | GTCCCACGAAGATCCCGAAGTTAAGTTCAATTGGTATGTTGAT |
| | | | GGTGTAGAGGTACACAATGCTAAGACTAAACCTCGCGAGGAG |
| | | | CAGTACGCCTCGACCTATCGTGTCGTGAGCGTTCTGACCGTCC |
| | | | TTCACCAAGATTGGCTTAACGGCAAAGAATATAAGTGCAAGG |
| | | | TAAGCAATAAAGCACTTCCGGCCCCAATCGAGAAAACCATTTC |
| | | | CAAGGCCAAAGGTCAACCAAGAGAACCCCAGGTGTATACTCT |
| | | | TCCGCCTTCTCGTGAGGAAATGACTAAAAATCAAGTATCCCTT |
| | | | ACGTGTCTGGTTAAAGGTTTTTATCCTAGCGATATTGCTGTTGA |
| | | | ATGGGAATCGAACGGTCAGCCGGAGAATAATTATAAAACAAC |
| | | | GCCACCCGTCCTGGATAGCGACGGCTCATTTTTTCTGTATAGC |
| | | | AAACTGACTGTAGATAAATCACGGTGGCAGCAGGGCAATGTA |
| | | | TTCAGTTGCTCCGTTATGCATGAAGCGTTACATAATCACTACA |
| | | | CGCAGAAATCTCTTAGTCTTTCACCCGGT |
| 1362 | ATX-P-3384 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCAGTGATAACTGGTGGAATTGGGTCCGCCAGCCCCCAGG |
| | | | GAAGGGGCTGGAGTGGCTTGGAAAAATCTATCATAGTGGGAA |
| | | | CACCGACTACAACCCGTCCCTCAAGAGTCGAGTCACCATATCA |
| | | | GTAGACAAGTCCAAGAATCAGTTCTCCCTGAGGCTGAGTTCTG |
| | | | TGACCGCCGCGGACACGGCCGTGTATTACTGTGCGAGAGGTG |
| | | | GAACTGAAACTACGGGGTACATGGACGTCTGGGGCAAAGGGA |
| | | | CCACGGTCACCGTCTCCTCAGCTAGCACTAAAGGGCCTTCTGT |
| | | | ATTTCCCTTGGCCCCGTCCAGCAAATCGACCTCGGGAGGGACA |
| | | | GCCGCCCTGGGTTGCCTTGTGAAAGATTATTTCCCTGAGCCAG |
| | | | TTACCGTAAGTTGGAACAGTGGGGCGCTGACAAGTGGTGTGC |
| | | | ACACGTTTCCTGCCGTCCTGCAATCATCGGGCTTGTATAGCCTC |
| | | | AGCTCTGTGGTCACTGTCCCAAGTTCATCGCTGGGCACTCAGA |
| | | | CGTATATTTGCAATGTGAACCACAAACCTTCAAATACAAAAGT |
| | | | GGATAAACGCGTAGAACCGAAATCGTGTGATAAAACTCACAC |
| | | | ATGCCCGCCATGCCCGGCACCTGAACTGCTTGGTGGTCCCAGC |
| | | | GTGTTCCTGTTCCCGCCGAAGCCTAAAGATACTCTAATGATCA |
| | | | GCCGTACGCCAGAGGTGACATGTGTCGTGGTTGACGTGTCCCA |
| | | | CGAAGATCCCGAAGTTAAGTTCAATTGGTATGTTGATGGTGTA |
| | | | GAGGTACACAATGCTAAGACTAAACCTCGCGAGGAGCAGTAC |
| | | | GCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1363 | ATX-P-3386 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCAATGATAACTGGTGGAGTTGGGTCCGCCAGCCCCCAGG |
| | | | GAAGGGGCTGGAATGGATTGGGAAAATCTATCATAGTGGGTT |
| | | | CACCAACTACAACCCGTCCCTCAAGAGTCGAGTCACCATATCA |
| | | | GTAGACAAGTCCAAGAACCAGTTCTCCCTGAAGCTGAACTCTG |
| | | | TGACCGCCGCGGACACGGCCGTGTATTACTGTGCGAGAGGGG |
| | | | GGGATAACTTTATGGAGGGGGAGGGGTTAGGGACCTGGGGCC |
| | | | CGGGAACCCTGGTCACCGTCTCCTCAGCTAGCACTAAAGGGCC |
| | | | TTCTGTATTTCCCTTGGCCCCGTCCAGCAAATCGACCTCGGGA |
| | | | GGGACAGCCGCCCTGGGTTGCCTTGTGAAAGATTATTTCCCTG |
| | | | AGCCAGTTACCGTAAGTTGGAACAGTGGGGCGCTGACAAGTG |
| | | | GTGTGCACACGTTTCCTGCCGTCCTGCAATCATCGGGCTTGTAT |
| | | | AGCCTCAGCTCTGTGGTCACTGTCCCAAGTTCATCGCTGGGCA |
| | | | CTCAGACGTATATTTGCAATGTGAACCACAAACCTTCAAATAC |
| | | | AAAAGTGGATAAACGCGTAGAACCGAAATCGTGTGATAAAAC |
| | | | TCACACATGCCCGCCATGCCCGGCACCTGAACTGCTTGGTGGT |
| | | | CCCAGCGTGTTCCTGTTCCCGCCGAAGCCTAAAGATACTCTAA |
| | | | TGATCAGCCGTACGCCAGAGGTGACATGTGTCGTGGTTGACGT |
| | | | GTCCCACGAAGATCCCGAAGTTAAGTTCAATTGGTATGTTGAT |
| | | | GGTGTAGAGGTACACAATGCTAAGACTAAACCTCGCGAGGAG |
| | | | CAGTACGCCTCGACCTATCGTGTCGTGAGCGTTCTGACCGTCC |
| | | | TTCACCAAGATTGGCTTAACGGCAAAGAATATAAGTGCAAGG |
| | | | TAAGCAATAAAGCACTTCCGGCCCCAATCGAGAAAACCATTTC |
| | | | CAAGGCCAAAGGTCAACCAAGAGAACCCCAGGTGTATACTCT |
| | | | TCCGCCTTCTCGTGAGGAAATGACTAAAAATCAAGTATCCCTT |
| | | | ACGTGTCTGGTTAAAGGTTTTTATCCTAGCGATATTGCTGTTGA |
| | | | ATGGGAATCGAACGGTCAGCCGGAGAATAATTATAAAACAAC |
| | | | GCCACCCGTCCTGGATAGCGACGGCTCATTTTTTCTGTATAGC |
| | | | AAACTGACTGTAGATAAATCACGGTGGCAGCAGGGCAATGTA |
| | | | TTCAGTTGCTCCGTTATGCATGAAGCGTTACATAATCACTACA |
| | | | CGCAGAAATCTCTTAGTCTTTCACCCGGT |
| 1364 | ATX-P-3387 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCAATGATAACTGGTGGAATTGGGTCCGCCAGTCCCCAGG |
| | | | GAAGGGGCTGGAGTGGATTGGAAAAATCTATCATAGTGGGTT |
| | | | CACCGACTACAACCCGTCCCTCAAGAGTCGAGTCACCATATCA |
| | | | GTAGACAAGTCCAAGAACCAGTTCTCCCTGAAGCTGAGCTCTG |
| | | | TGACCGCCGCGGACACGGCCGTGTATTATTGTGCGAGAGGAA |
| | | | ACCATAACTATTTGTGGATTGGGTATTTGGGGACCTGGGGCCC |
| | | | GGGAACCCTGGTCACCGTCTCCTCAGCTAGCACTAAAGGGCCT |
| | | | TCTGTATTTCCCTTGGCCCCGTCCAGCAAATCGACCTCGGGAG |
| | | | GGACAGCCGCCCTGGGTTGCCTTGTGAAAGATTATTTCCCTGA |
| | | | GCCAGTTACCGTAAGTTGGAACAGTGGGGCGCTGACAAGTGG |
| | | | TGTGCACACGTTTCCTGCCGTCCTGCAATCATCGGGCTTGTATA |
| | | | GCCTCAGCTCTGTGGTCACTGTCCCAAGTTCATCGCTGGGCAC |
| | | | TCAGACGTATATTTGCAATGTGAACCACAAACCTTCAAATACA |
| | | | AAAGTGGATAAACGCGTAGAACCGAAATCGTGTGATAAAACT |
| | | | CACACATGCCCGCCATGCCCGGCACCTGAACTGCTTGGTGGTC |
| | | | CCAGCGTGTTCCTGTTCCCGCCGAAGCCTAAAGATACTCTAAT |
| | | | GATCAGCCGTACGCCAGAGGTGACATGTGTCGTGGTTGACGTG |
| | | | TCCCACGAAGATCCCGAAGTTAAGTTCAATTGGTATGTTGATG |
| | | | GTGTAGAGGTACACAATGCTAAGACTAAACCTCGCGAGGAGC |
| | | | AGTACGCCTCGACCTATCGTGTCGTGAGCGTTCTGACCGTCCT |
| | | | TCACCAAGATTGGCTTAACGGCAAAGAATATAAGTGCAAGGT |
| | | | AAGCAATAAAGCACTTCCGGCCCCAATCGAGAAAACCATTTCC |
| | | | AAGGCCAAAGGTCAACCAAGAGAACCCCAGGTGTATACTCTT |
| | | | CCGCCTTCTCGTGAGGAAATGACTAAAAATCAAGTATCCCTTA |
| | | | CGTGTCTGGTTAAAGGTTTTTATCCTAGCGATATTGCTGTTGAA |
| | | | TGGGAATCGAACGGTCAGCCGGAGAATAATTATAAAACAACG |
| | | | CCACCCGTCCTGGATAGCGACGGCTCATTTTTTCTGTATAGCA |
| | | | AACTGACTGTAGATAAATCACGGTGGCAGCAGGGCAATGTAT |
| | | | TCAGTTGCTCCGTTATGCATGAAGCGTTACATAATCACTACAC |
| | | | GCAGAAATCTCTTAGTCTTTCACCCGGT |
| 1365 | ATX-P-3389 | HC IgG1 Fc nt | CAGGTGCAGCTGCAGGAGTCGGGCCCAGGACTGGTGAAGCCT |
| | | | TCGGGGACCCTGTCCCTCATCTGCGCTGTCTCTGGTGGCTCCAT |
| | | | CAGTAGTAGTAATTGGTGGAGTTGGGTCCGCCAGCCCCCAGGG |
| | | | AAGGGGCTGGAGTGGATTGGGAGAATCTATCATAGTGGGCAC |
| | | | ACCAACTACAACCCGTCCCTCAAGAGTCGAGTCACCATATCAG |
| | | | TAGACAAGTCCAAGAACCAGTTCTCCCTGAAGCTGACCTCTGT |
| | | | GACCGCCGCGGACACGGCCGTGTATTACTGTGCGAGAGGGAG |
| | | | GGACTTAACTGGATACTTTGACTACTGGGGCCAGGGAACCCTG |
| | | | GTCACCGTCTCCTCAGCTAGCACTAAAGGGCCTTCTGTATTTCC |
| | | | CTTGGCCCCGTCCAGCAAATCGACCTCGGGAGGGACAGCCGC |
| | | | CCTGGGTTGCCTTGTGAAAGATTATTTCCCTGAGCCAGTTACC |
| | | | GTAAGTTGGAACAGTGGGGCGCTGACAAGTGGTGTGCACACG |
| | | | TTTCCTGCCGTCCTGCAATCATCGGGCTTGTATAGCCTCAGCTC |
| | | | TGTGGTCACTGTCCCAAGTTCATCGCTGGGCACTCAGACGTAT |
| | | | ATTTGCAATGTGAACCACAAACCTTCAAATACAAAAGTGGATA |
| | | | AACGCGTAGAACCGAAATCGTGTGATAAAACTCACACATGCC |
| | | | CGCCATGCCCGGCACCTGAACTGCTTGGTGGTCCCAGCGTGTT |
| | | | CCTGTTCCCGCCGAAGCCTAAAGATACTCTAATGATCAGCCGT |
| | | | ACGCCAGAGGTGACATGTGTCGTGGTTGACGTGTCCCACGAAG |
| | | | ATCCCGAAGTTAAGTTCAATTGGTATGTTGATGGTGTAGAGGT |
| | | | ACACAATGCTAAGACTAAACCTCGCGAGGAGCAGTACGCCTC |
| | | | GACCTATCGTGTCGTGAGCGTTCTGACCGTCCTTCACCAAGAT |
| | | | TGGCTTAACGGCAAAGAATATAAGTGCAAGGTAAGCAATAAA |
| | | | GCACTTCCGGCCCCAATCGAGAAAACCATTTCCAAGGCCAAA |
| | | | GGTCAACCAAGAGAACCCCAGGTGTATACTCTTCCGCCTTCTC |
| | | | GTGAGGAAATGACTAAAAATCAAGTATCCCTTACGTGTCTGGT |
| | | | TAAAGGTTTTTATCCTAGCGATATTGCTGTTGAATGGGAATCG |
| | | | AACGGTCAGCCGGAGAATAATTATAAAACAACGCCACCCGTC |
| | | | CTGGATAGCGACGGCTCATTTTTTCTGTATAGCAAACTGACTG |
| | | | TAGATAAATCACGGTGGCAGCAGGGCAATGTATTCAGTTGCTC |
| | | | CGTTATGCATGAAGCGTTACATAATCACTACACGCAGAAATCT |
| | | | CTTAGTCTTTCACCCGGT |
| 1366 | ATX-P-3391 | HC IgG1 Fc nt | CAGGTGCAGCTGCAGGAGTCGGGCCCAGGACTGGTGAAACCT |
| | | | TCGGGGACCCTGTCCCTCACCTGCGCTGTCTCTGGTGGCTCCAT |
| | | | CAGTAATGATAACTGGTGGAGTTGGGTCCGCCAGTCCCCCGGG |
| | | | AAGGGGCTGGAGTGGATTGGGAAAATCTATCATAGTGAGTTC |
| | | | ACCAACTACAACCCGTCCCTCAAGAGTCGAGTCACCATATCAG |
| | | | TAGACAAGTCCAAGAACCAGTTCTCCCTGGAGCTGAGCTCTGT |
| | | | GACCGCCGCGGACACGGCCGTATATTACTGTGCGAGAGGGGT |
| | | | CGATTTTTGGAGTGGTTCCCACATGGACGTCTGGGGCAAAGGG |
| | | | ACCACGGTCACCGTCTCCTCAGCTAGCACTAAAGGGCCTTCTG |
| | | | TATTTCCCTTGGCCCCGTCCAGCAAATCGACCTCGGGAGGGAC |
| | | | AGCCGCCCTGGGTTGCCTTGTGAAAGATTATTTCCCTGAGCCA |
| | | | GTTACCGTAAGTTGGAACAGTGGGGCGCTGACAAGTGGTGTG |
| | | | CACACGTTTCCTGCCGTCCTGCAATCATCGGGCTTGTATAGCCT |
| | | | CAGCTCTGTGGTCACTGTCCCAAGTTCATCGCTGGGCACTCAG |
| | | | ACGTATATTTGCAATGTGAACCACAAACCTTCAAATACAAAAG |
| | | | TGGATAAACGCGTAGAACCGAAATCGTGTGATAAAACTCACA |
| | | | CATGCCCGCCATGCCCGGCACCTGAACTGCTTGGTGGTCCCAG |
| | | | CGTGTTCCTGTTCCCGCCGAAGCCTAAAGATACTCTAATGATC |
| | | | AGCCGTACGCCAGAGGTGACATGTGTCGTGGTTGACGTGTCCC |
| | | | ACGAAGATCCCGAAGTTAAGTTCAATTGGTATGTTGATGGTGT |
| | | | AGAGGTACACAATGCTAAGACTAAACCTCGCGAGGAGCAGTA |
| | | | CGCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1367 | ATX-P-3392 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGTAATGATAACTGGTGGAGTTGGGTCCGCCAGTCCCCCGGG |
| | | | AAGGGGCTGGAGTGGATTGGGAAAATCTATCATAGTGAGTTC |
| | | | ACCAACTACAACCCGTCCCTCAAGAGTCGAGTCACCATATCAG |
| | | | TAGACAAGTCTAAGAACCAGTTTTCCCTGGAACTGAGCTCTGT |
| | | | GACCGCCGCGGACACGGCCGTGTATTTCTGTGCGCGAGAAGG |
| | | | ACTGGAACTACCCCACATGGACGTCTGGGGCAAAGGGACCAC |
| | | | GGTCACCGTCTCCTCAGCTAGCACTAAAGGGCCTTCTGTATTT |
| | | | CCCTTGGCCCCGTCCAGCAAATCGACCTCGGGAGGGACAGCC |
| | | | GCCCTGGGTTGCCTTGTGAAAGATTATTTCCCTGAGCCAGTTA |
| | | | CCGTAAGTTGGAACAGTGGGGCGCTGACAAGTGGTGTGCACA |
| | | | CGTTTCCTGCCGTCCTGCAATCATCGGGCTTGTATAGCCTCAGC |
| | | | TCTGTGGTCACTGTCCCAAGTTCATCGCTGGGCACTCAGACGT |
| | | | ATATTTGCAATGTGAACCACAAACCTTCAAATACAAAAGTGGA |
| | | | TAAACGCGTAGAACCGAAATCGTGTGATAAAACTCACACATG |
| | | | CCCGCCATGCCCGGCACCTGAACTGCTTGGTGGTCCCAGCGTG |
| | | | TTCCTGTTCCCGCCGAAGCCTAAAGATACTCTAATGATCAGCC |
| | | | GTACGCCAGAGGTGACATGTGTCGTGGTTGACGTGTCCCACGA |
| | | | AGATCCCGAAGTTAAGTTCAATTGGTATGTTGATGGTGTAGAG |
| | | | GTACACAATGCTAAGACTAAACCTCGCGAGGAGCAGTACGCC |
| | | | TCGACCTATCGTGTCGTGAGCGTTCTGACCGTCCTTCACCAAG |
| | | | ATTGGCTTAACGGCAAAGAATATAAGTGCAAGGTAAGCAATA |
| | | | AAGCACTTCCGGCCCCAATCGAGAAAACCATTTCCAAGGCCA |
| | | | AAGGTCAACCAAGAGAACCCCAGGTGTATACTCTTCCGCCTTC |
| | | | TCGTGAGGAAATGACTAAAAATCAAGTATCCCTTACGTGTCTG |
| | | | GTTAAAGGTTTTTATCCTAGCGATATTGCTGTTGAATGGGAAT |
| | | | CGAACGGTCAGCCGGAGAATAATTATAAAACAACGCCACCCG |
| | | | TCCTGGATAGCGACGGCTCATTTTTTCTGTATAGCAAACTGAC |
| | | | TGTAGATAAATCACGGTGGCAGCAGGGCAATGTATTCAGTTGC |
| | | | TCCGTTATGCATGAAGCGTTACATAATCACTACACGCAGAAAT |
| | | | CTCTTAGTCTTTCACCCGGT |
| 1368 | ATX-P-3395 | HC IgG1 Fc nt | GAGGTGCAGCTGGTGGAGTCCGGGGGAGGCGTGGTCAAGCCT |
| | | | GGGGGGTCCCTGAGACTCTCCTGTGCAGCCTCTGGATTCACCT |
| | | | TCAGTAGCTACTGGATGCACTGGGTCCGCCAAGCTCCAGGGAA |
| | | | GGGGCTGGTGTGGGTCTCACGTATTAATAGTGATGGAAGTAGC |
| | | | ACAAGCTACGCGGACTCCGTGCAGGGCCGATTCACCATCTCCA |
| | | | GAGACATCGCCAAGAACACGCTGTATCTGCAAATGAACAGTC |
| | | | TGAGAGCCGAGGACACGGCTGTGTATTACTGTGCAAGAGGGC |
| | | | AGCACCTGGTTCAGGACTATTGGGGCCAGGGAACCCTGGTCAC |
| | | | CGTCTCTTCAGCTAGCACTAAAGGGCCTTCTGTATTTCCCTTGG |
| | | | CCCCGTCCAGCAAATCGACCTCGGGAGGGACAGCCGCCCTGG |
| | | | GTTGCCTTGTGAAAGATTATTTCCCTGAGCCAGTTACCGTAAG |
| | | | TTGGAACAGTGGGGCGCTGACAAGTGGTGTGCACACGTTTCCT |
| | | | GCCGTCCTGCAATCATCGGGCTTGTATAGCCTCAGCTCTGTGG |
| | | | TCACTGTCCCAAGTTCATCGCTGGGCACTCAGACGTATATTTG |
| | | | CAATGTGAACCACAAACCTTCAAATACAAAAGTGGATAAACG |
| | | | CGTAGAACCGAAATCGTGTGATAAAACTCACACATGCCCGCC |
| | | | ATGCCCGGCACCTGAACTGCTTGGTGGTCCCAGCGTGTTCCTG |
| | | | TTCCCGCCGAAGCCTAAAGATACTCTAATGATCAGCCGTACGC |
| | | | CAGAGGTGACATGTGTCGTGGTTGACGTGTCCCACGAAGATCC |
| | | | CGAAGTTAAGTTCAATTGGTATGTTGATGGTGTAGAGGTACAC |
| | | | AATGCTAAGACTAAACCTCGCGAGGAGCAGTACGCCTCGACC |
| | | | TATCGTGTCGTGAGCGTTCTGACCGTCCTTCACCAAGATTGGC |
| | | | TTAACGGCAAAGAATATAAGTGCAAGGTAAGCAATAAAGCAC |
| | | | TTCCGGCCCCAATCGAGAAAACCATTTCCAAGGCCAAAGGTCA |
| | | | ACCAAGAGAACCCCAGGTGTATACTCTTCCGCCTTCTCGTGAG |
| | | | GAAATGACTAAAAATCAAGTATCCCTTACGTGTCTGGTTAAAG |
| | | | GTTTTTATCCTAGCGATATTGCTGTTGAATGGGAATCGAACGG |
| | | | TCAGCCGGAGAATAATTATAAAACAACGCCACCCGTCCTGGAT |
| | | | AGCGACGGCTCATTTTTTCTGTATAGCAAACTGACTGTAGATA |
| | | | AATCACGGTGGCAGCAGGGCAATGTATTCAGTTGCTCCGTTAT |
| | | | GCATGAAGCGTTACATAATCACTACACGCAGAAATCTCTTAGT |
| | | | CTTTCACCCGGT |
| 1369 | ATX-P-3396 | HC IgG1 Fc nt | CAGGTGCAGCTGGTGCAGTCTGGGGCTGAGGTGAAGAACCCT |
| | | | GGGGCCTCAGTGAAGGTCTCCTGCAAGGCTTCTGGATACACCT |
| | | | TCACCGGCTACTATATGCACTGGGTGCGACAGGCCCCTGGACA |
| | | | AGGGCTTGAGTGGATGGGATGGATCAACCCTAACAGTGGTGG |
| | | | CACAAACTATGCACAGAAGTTTCAGGGCAGGGTCACCATGAC |
| | | | CAGGGACACGTCCATCAGCACAGCCTACATGGAGCTGAGCAG |
| | | | GCTGAGATCTGACGACACGGCCGTGTATTACTGTGCGAGAGA |
| | | | ACGGGGCGACTATGATGGCGATGCTTTTGATATCTGGGGACAA |
| | | | GGGACAATGGTCACCGTCTCTTCAGCTAGCACTAAAGGGCCTT |
| | | | CTGTATTTCCCTTGGCCCCGTCCAGCAAATCGACCTCGGGAGG |
| | | | GACAGCCGCCCTGGGTTGCCTTGTGAAAGATTATTTCCCTGAG |
| | | | CCAGTTACCGTAAGTTGGAACAGTGGGGCGCTGACAAGTGGT |
| | | | GTGCACACGTTTCCTGCCGTCCTGCAATCATCGGGCTTGTATA |
| | | | GCCTCAGCTCTGTGGTCACTGTCCCAAGTTCATCGCTGGGCAC |
| | | | TCAGACGTATATTTGCAATGTGAACCACAAACCTTCAAATACA |
| | | | AAAGTGGATAAACGCGTAGAACCGAAATCGTGTGATAAAACT |
| | | | CACACATGCCCGCCATGCCCGGCACCTGAACTGCTTGGTGGTC |
| | | | CCAGCGTGTTCCTGTTCCCGCCGAAGCCTAAAGATACTCTAAT |
| | | | GATCAGCCGTACGCCAGAGGTGACATGTGTCGTGGTTGACGTG |
| | | | TCCCACGAAGATCCCGAAGTTAAGTTCAATTGGTATGTTGATG |
| | | | GTGTAGAGGTACACAATGCTAAGACTAAACCTCGCGAGGAGC |
| | | | AGTACGCCTCGACCTATCGTGTCGTGAGCGTTCTGACCGTCCT |
| | | | TCACCAAGATTGGCTTAACGGCAAAGAATATAAGTGCAAGGT |
| | | | AAGCAATAAAGCACTTCCGGCCCCAATCGAGAAAACCATTTCC |
| | | | AAGGCCAAAGGTCAACCAAGAGAACCCCAGGTGTATACTCTT |
| | | | CCGCCTTCTCGTGAGGAAATGACTAAAAATCAAGTATCCCTTA |
| | | | CGTGTCTGGTTAAAGGTTTTTATCCTAGCGATATTGCTGTTGAA |
| | | | TGGGAATCGAACGGTCAGCCGGAGAATAATTATAAAACAACG |
| | | | CCACCCGTCCTGGATAGCGACGGCTCATTTTTTCTGTATAGCA |
| | | | AACTGACTGTAGATAAATCACGGTGGCAGCAGGGCAATGTAT |
| | | | TCAGTTGCTCCGTTATGCATGAAGCGTTACATAATCACTACAC |
| | | | GCAGAAATCTCTTAGTCTTTCACCCGGT |
| 1370 | ATX-P-3397 | HC IgG1 Fc nt | GAGGTGCAGCTGGTGGAGTCTGGGGGAGGCTTGGTCCAGCCT |
| | | | GGGGGGTCCCTGAGACTCTCCTGTGCAGCCTCTGGATTCACCT |
| | | | TTAGTAACTATTACATGAACTGGGTCCGCCAGGCTCCAGGGAA |
| | | | GGGGCTGGAGTGGGTGGCCAACATAAAGCAAGATGGAAGTGA |
| | | | GAAATACTATGTGGACTCTGTGAAGGGCCGCTTCACCATCTCC |
| | | | AGAGACAACGCCAAGAACTCACTGTATCTCCAAATGAACAGC |
| | | | CTGAGAGCCGAGGACACGGCTGTGTATTACTGTGCGAGAGAT |
| | | | GGGTACTACAACTTCGATGCTTTTGATATCTGGGGACAAGGGA |
| | | | CAATGGTCACCGTCTCTTCAGCTAGCACTAAAGGGCCTTCTGT |
| | | | ATTTCCCTTGGCCCCGTCCAGCAAATCGACCTCGGGAGGGACA |
| | | | GCCGCCCTGGGTTGCCTTGTGAAAGATTATTTCCCTGAGCCAG |
| | | | TTACCGTAAGTTGGAACAGTGGGGCGCTGACAAGTGGTGTGC |
| | | | ACACGTTTCCTGCCGTCCTGCAATCATCGGGCTTGTATAGCCTC |
| | | | AGCTCTGTGGTCACTGTCCCAAGTTCATCGCTGGGCACTCAGA |
| | | | CGTATATTTGCAATGTGAACCACAAACCTTCAAATACAAAAGT |
| | | | GGATAAACGCGTAGAACCGAAATCGTGTGATAAAACTCACAC |
| | | | ATGCCCGCCATGCCCGGCACCTGAACTGCTTGGTGGTCCCAGC |
| | | | GTGTTCCTGTTCCCGCCGAAGCCTAAAGATACTCTAATGATCA |
| | | | GCCGTACGCCAGAGGTGACATGTGTCGTGGTTGACGTGTCCCA |
| | | | CGAAGATCCCGAAGTTAAGTTCAATTGGTATGTTGATGGTGTA |
| | | | GAGGTACACAATGCTAAGACTAAACCTCGCGAGGAGCAGTAC |
| | | | GCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1371 | ATX-P-3405 | HC IgG1 Fc nt | GAGGTGCAGCTGGTGGAGTCTGGGGGAGGCTTGGTACAGCCT |
| | | | GGGGGGTCCCTGAGACTCTCCTGTGCAGCCTCTGGATTCACCT |
| | | | TCAGTAGCTATAGCATGAACTGGGTCCGCCAGGCTCCAGGGA |
| | | | AGGGGCTGGAGTGGGTCTCATCCATTAGTAGTAGTAGTAGTTA |
| | | | CATATACTACGCAGACTCAGTGAAGGGCCGATTCACCATCTCC |
| | | | AGAGACAACGCCAAGAACTCACTGTATCTGCAAATGAACAGC |
| | | | CTGAGAGCCGAGGACACGGCCGTGTATTACTGTGCGAGAGAA |
| | | | GGGTATGATAACTGGAACTCTGCTTGTGATATCTGGGGACAAG |
| | | | GGACAATGGTCACCGTCTCTTCAGCTAGCACTAAAGGGCCTTC |
| | | | TGTATTTCCCTTGGCCCCGTCCAGCAAATCGACCTCGGGAGGG |
| | | | ACAGCCGCCCTGGGTTGCCTTGTGAAAGATTATTTCCCTGAGC |
| | | | CAGTTACCGTAAGTTGGAACAGTGGGGCGCTGACAAGTGGTG |
| | | | TGCACACGTTTCCTGCCGTCCTGCAATCATCGGGCTTGTATAG |
| | | | CCTCAGCTCTGTGGTCACTGTCCCAAGTTCATCGCTGGGCACT |
| | | | CAGACGTATATTTGCAATGTGAACCACAAACCTTCAAATACAA |
| | | | AAGTGGATAAACGCGTAGAACCGAAATCGTGTGATAAAACTC |
| | | | ACACATGCCCGCCATGCCCGGCACCTGAACTGCTTGGTGGTCC |
| | | | CAGCGTGTTCCTGTTCCCGCCGAAGCCTAAAGATACTCTAATG |
| | | | ATCAGCCGTACGCCAGAGGTGACATGTGTCGTGGTTGACGTGT |
| | | | CCCACGAAGATCCCGAAGTTAAGTTCAATTGGTATGTTGATGG |
| | | | TGTAGAGGTACACAATGCTAAGACTAAACCTCGCGAGGAGCA |
| | | | GTACGCCTCGACCTATCGTGTCGTGAGCGTTCTGACCGTCCTTC |
| | | | ACCAAGATTGGCTTAACGGCAAAGAATATAAGTGCAAGGTAA |
| | | | GCAATAAAGCACTTCCGGCCCCAATCGAGAAAACCATTTCCAA |
| | | | GGCCAAAGGTCAACCAAGAGAACCCCAGGTGTATACTCTTCC |
| | | | GCCTTCTCGTGAGGAAATGACTAAAAATCAAGTATCCCTTACG |
| | | | TGTCTGGTTAAAGGTTTTTATCCTAGCGATATTGCTGTTGAATG |
| | | | GGAATCGAACGGTCAGCCGGAGAATAATTATAAAACAACGCC |
| | | | ACCCGTCCTGGATAGCGACGGCTCATTTTTTCTGTATAGCAAA |
| | | | CTGACTGTAGATAAATCACGGTGGCAGCAGGGCAATGTATTCA |
| | | | GTTGCTCCGTTATGCATGAAGCGTTACATAATCACTACACGCA |
| | | | GAAATCTCTTAGTCTTTCACCCGGT |
| 1372 | ATX-P-3406 | HC IgG1 Fc nt | CAGATCACCTTGAAGGAGTCTGGTCCTACGCTGGTGAAACCCA |
| | | | CACAGACCCTCACGCTGACCTGCACCTTCTCTGGGTTTTCACTC |
| | | | AGCACTAGTGGAGTGGGTGTGGGCTGGATCCGTCAGCCCCCA |
| | | | GGAAAGGCCCTGGAGTGGCTTGCACTCATTTATTGGAATGGTG |
| | | | ATGAGCGCTACAGCCCATCTCTGAAGAGCAGGCTCACCATCAC |
| | | | CAAGGACACCTCCAAAAACCAAGTGGTCCTTACAATGACCAA |
| | | | CATGGACCCTGTGGACACAGCCACATATTACTGTGCACACAGT |
| | | | CCGAACTGGGGATATGAAGCTTTTGATATCTGGGGACAAGGG |
| | | | ACAATGGTCACCGTCTCTTCAGCTAGCACTAAAGGGCCTTCTG |
| | | | TATTTCCCTTGGCCCCGTCCAGCAAATCGACCTCGGGAGGGAC |
| | | | AGCCGCCCTGGGTTGCCTTGTGAAAGATTATTTCCCTGAGCCA |
| | | | GTTACCGTAAGTTGGAACAGTGGGGCGCTGACAAGTGGTGTG |
| | | | CACACGTTTCCTGCCGTCCTGCAATCATCGGGCTTGTATAGCCT |
| | | | CAGCTCTGTGGTCACTGTCCCAAGTTCATCGCTGGGCACTCAG |
| | | | ACGTATATTTGCAATGTGAACCACAAACCTTCAAATACAAAAG |
| | | | TGGATAAACGCGTAGAACCGAAATCGTGTGATAAAACTCACA |
| | | | CATGCCCGCCATGCCCGGCACCTGAACTGCTTGGTGGTCCCAG |
| | | | CGTGTTCCTGTTCCCGCCGAAGCCTAAAGATACTCTAATGATC |
| | | | AGCCGTACGCCAGAGGTGACATGTGTCGTGGTTGACGTGTCCC |
| | | | ACGAAGATCCCGAAGTTAAGTTCAATTGGTATGTTGATGGTGT |
| | | | AGAGGTACACAATGCTAAGACTAAACCTCGCGAGGAGCAGTA |
| | | | CGCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1373 | ATX-P-3407 | HC IgG1 Fc nt | GAGGTGCAGCTGGTGGAGTCTGGGGGAGGCCTGGTCCCGCCT |
| | | | GGGGGGTCCCTGAGACTCTCCTGTGCAGCCTCTGGATTCACCT |
| | | | TCAGTAGCTATTGCATGAACTGGGTCCGCCAGGCTCCAGGGAA |
| | | | GGGGCTGGAGTGGGTCTCATCCATTAATAGTGATAGTAGTTAC |
| | | | ATATACTACGCGGACTCAGTGAAGGGCCGATTCACCATCTCCA |
| | | | GAGACAACGCCAAGAACTCACTGTATCTGCAAATGAACAGCC |
| | | | TGAGAGCCGAGGACACGGCTGTGTATTACTGTGCGAGAGAGG |
| | | | GATACTACAACTGGAACGGAGGGAATGACTACTGGGGCCAGG |
| | | | GAACCCTGGTCACCGTCTCCTCAGCTAGCACTAAAGGGCCTTC |
| | | | TGTATTTCCCTTGGCCCCGTCCAGCAAATCGACCTCGGGAGGG |
| | | | ACAGCCGCCCTGGGTTGCCTTGTGAAAGATTATTTCCCTGAGC |
| | | | CAGTTACCGTAAGTTGGAACAGTGGGGCGCTGACAAGTGGTG |
| | | | TGCACACGTTTCCTGCCGTCCTGCAATCATCGGGCTTGTATAG |
| | | | CCTCAGCTCTGTGGTCACTGTCCCAAGTTCATCGCTGGGCACT |
| | | | CAGACGTATATTTGCAATGTGAACCACAAACCTTCAAATACAA |
| | | | AAGTGGATAAACGCGTAGAACCGAAATCGTGTGATAAAACTC |
| | | | ACACATGCCCGCCATGCCCGGCACCTGAACTGCTTGGTGGTCC |
| | | | CAGCGTGTTCCTGTTCCCGCCGAAGCCTAAAGATACTCTAATG |
| | | | ATCAGCCGTACGCCAGAGGTGACATGTGTCGTGGTTGACGTGT |
| | | | CCCACGAAGATCCCGAAGTTAAGTTCAATTGGTATGTTGATGG |
| | | | TGTAGAGGTACACAATGCTAAGACTAAACCTCGCGAGGAGCA |
| | | | GTACGCCTCGACCTATCGTGTCGTGAGCGTTCTGACCGTCCTTC |
| | | | ACCAAGATTGGCTTAACGGCAAAGAATATAAGTGCAAGGTAA |
| | | | GCAATAAAGCACTTCCGGCCCCAATCGAGAAAACCATTTCCAA |
| | | | GGCCAAAGGTCAACCAAGAGAACCCCAGGTGTATACTCTTCC |
| | | | GCCTTCTCGTGAGGAAATGACTAAAAATCAAGTATCCCTTACG |
| | | | TGTCTGGTTAAAGGTTTTTATCCTAGCGATATTGCTGTTGAATG |
| | | | GGAATCGAACGGTCAGCCGGAGAATAATTATAAAACAACGCC |
| | | | ACCCGTCCTGGATAGCGACGGCTCATTTTTTCTGTATAGCAAA |
| | | | CTGACTGTAGATAAATCACGGTGGCAGCAGGGCAATGTATTCA |
| | | | GTTGCTCCGTTATGCATGAAGCGTTACATAATCACTACACGCA |
| | | | GAAATCTCTTAGTCTTTCACCCGGT |
| 1374 | ATX-P-3408 | HC IgG1 Fc nt | GAGGTGCAGCTGGTGGAGTCCGGGGGAGGCTTAGTTCAGCCT |
| | | | GGGGGGTCCCTGAGACTCTCCTGTGCAGCCTCTGGATTCACCT |
| | | | TCAGTAGCTATTGGATGCACTGGGTCCGCCAAGCTCCAGGGAA |
| | | | GGGGCTGGTGTGGGTCTCACATATTAATAGTGATGGGAGTAGC |
| | | | ACAAACTACGCGGACTCCGTGAAGGGCCGATTCACCATCTCCA |
| | | | GAGACAACGCCAAGAACACGCTGTATCTGCAGATGAACAGTC |
| | | | TGAGATCCGAGGACACGGCTGTATATTACTGTGCAAGAGAGG |
| | | | GTTACTGGAACGGTGATACTTTTGATATCTGGGGACAAGGGAC |
| | | | AATGGTCACCGTCTCTTCAGCTAGCACTAAAGGGCCTTCTGTA |
| | | | TTTCCCTTGGCCCCGTCCAGCAAATCGACCTCGGGAGGGACAG |
| | | | CCGCCCTGGGTTGCCTTGTGAAAGATTATTTCCCTGAGCCAGT |
| | | | TACCGTAAGTTGGAACAGTGGGGCGCTGACAAGTGGTGTGCA |
| | | | CACGTTTCCTGCCGTCCTGCAATCATCGGGCTTGTATAGCCTCA |
| | | | GCTCTGTGGTCACTGTCCCAAGTTCATCGCTGGGCACTCAGAC |
| | | | GTATATTTGCAATGTGAACCACAAACCTTCAAATACAAAAGTG |
| | | | GATAAACGCGTAGAACCGAAATCGTGTGATAAAACTCACACA |
| | | | TGCCCGCCATGCCCGGCACCTGAACTGCTTGGTGGTCCCAGCG |
| | | | TGTTCCTGTTCCCGCCGAAGCCTAAAGATACTCTAATGATCAG |
| | | | CCGTACGCCAGAGGTGACATGTGTCGTGGTTGACGTGTCCCAC |
| | | | GAAGATCCCGAAGTTAAGTTCAATTGGTATGTTGATGGTGTAG |
| | | | AGGTACACAATGCTAAGACTAAACCTCGCGAGGAGCAGTACG |
| | | | CCTCGACCTATCGTGTCGTGAGCGTTCTGACCGTCCTTCACCA |
| | | | AGATTGGCTTAACGGCAAAGAATATAAGTGCAAGGTAAGCAA |
| | | | TAAAGCACTTCCGGCCCCAATCGAGAAAACCATTTCCAAGGCC |
| | | | AAAGGTCAACCAAGAGAACCCCAGGTGTATACTCTTCCGCCTT |
| | | | CTCGTGAGGAAATGACTAAAAATCAAGTATCCCTTACGTGTCT |
| | | | GGTTAAAGGTTTTTATCCTAGCGATATTGCTGTTGAATGGGAA |
| | | | TCGAACGGTCAGCCGGAGAATAATTATAAAACAACGCCACCC |
| | | | GTCCTGGATAGCGACGGCTCATTTTTTCTGTATAGCAAACTGA |
| | | | CTGTAGATAAATCACGGTGGCAGCAGGGCAATGTATTCAGTTG |
| | | | CTCCGTTATGCATGAAGCGTTACATAATCACTACACGCAGAAA |
| | | | TCTCTTAGTCTTTCACCCGGT |
| 1375 | ATX-P-3410 | HC IgG1 Fc nt | GAGGTGCAGCTGGTGGAGTCTGGGGGAGGCTTGGTACAGCCT |
| | | | GGGGGGTCCCTGAGACTCTCCTGTGCAGCCTCTGGATTCACCT |
| | | | TCAGTGCCTACGACATGCACTGGGTCCGCCAAACTCCAGGAAA |
| | | | AGGTCTGGAGTGGGTCTCAACTATTGGTACTGCTGGTGACACA |
| | | | TACTATCCAGACTCCGTGAAGGGCCGATTCACCATCTCCAGAG |
| | | | ATTTTGCCAGGAGGTCCTTGTATCTTCACATGAACAATCTGAG |
| | | | AGCCGGGGACACGGCTGTATATTACTGTGCAAGAGGGGGCAG |
| | | | CAGCAGCTGGTACGACTGGTACTTCGATCTCTGGGGCCGTGGC |
| | | | ACCCTGGTCACTGTCTCCTCAGCTAGCACTAAAGGGCCTTCTG |
| | | | TATTTCCCTTGGCCCCGTCCAGCAAATCGACCTCGGGAGGGAC |
| | | | AGCCGCCCTGGGTTGCCTTGTGAAAGATTATTTCCCTGAGCCA |
| | | | GTTACCGTAAGTTGGAACAGTGGGGCGCTGACAAGTGGTGTG |
| | | | CACACGTTTCCTGCCGTCCTGCAATCATCGGGCTTGTATAGCCT |
| | | | CAGCTCTGTGGTCACTGTCCCAAGTTCATCGCTGGGCACTCAG |
| | | | ACGTATATTTGCAATGTGAACCACAAACCTTCAAATACAAAAG |
| | | | TGGATAAACGCGTAGAACCGAAATCGTGTGATAAAACTCACA |
| | | | CATGCCCGCCATGCCCGGCACCTGAACTGCTTGGTGGTCCCAG |
| | | | CGTGTTCCTGTTCCCGCCGAAGCCTAAAGATACTCTAATGATC |
| | | | AGCCGTACGCCAGAGGTGACATGTGTCGTGGTTGACGTGTCCC |
| | | | ACGAAGATCCCGAAGTTAAGTTCAATTGGTATGTTGATGGTGT |
| | | | AGAGGTACACAATGCTAAGACTAAACCTCGCGAGGAGCAGTA |
| | | | CGCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1376 | ATX-P-3412 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCAGTAGTACCTGGTGGAGTTGGGTCCGCCAGCCCCCAGG |
| | | | GAAGGGGCTGGAGTGGATTGGGGAAATCTATCATAGTGGGAG |
| | | | CACCAACTATAACCCGTCCCTCAAGAGTCGAGTCACCATATCA |
| | | | CTAGACAAGTCCAAGAACCAGTTTTCCCTGAAGCTGAGCTCTG |
| | | | TGACCGCCGCGGACACGGCCGTGTATTACTGTGCGATTAGTTT |
| | | | ATACAACTGGAACGATGCTGCTTTTGATATCTGGGGACAAGGG |
| | | | ACAATGGTCACCGTCTCTTCAGCTAGCACTAAAGGGCCTTCTG |
| | | | TATTTCCCTTGGCCCCGTCCAGCAAATCGACCTCGGGAGGGAC |
| | | | AGCCGCCCTGGGTTGCCTTGTGAAAGATTATTTCCCTGAGCCA |
| | | | GTTACCGTAAGTTGGAACAGTGGGGCGCTGACAAGTGGTGTG |
| | | | CACACGTTTCCTGCCGTCCTGCAATCATCGGGCTTGTATAGCCT |
| | | | CAGCTCTGTGGTCACTGTCCCAAGTTCATCGCTGGGCACTCAG |
| | | | ACGTATATTTGCAATGTGAACCACAAACCTTCAAATACAAAAG |
| | | | TGGATAAACGCGTAGAACCGAAATCGTGTGATAAAACTCACA |
| | | | CATGCCCGCCATGCCCGGCACCTGAACTGCTTGGTGGTCCCAG |
| | | | CGTGTTCCTGTTCCCGCCGAAGCCTAAAGATACTCTAATGATC |
| | | | AGCCGTACGCCAGAGGTGACATGTGTCGTGGTTGACGTGTCCC |
| | | | ACGAAGATCCCGAAGTTAAGTTCAATTGGTATGTTGATGGTGT |
| | | | AGAGGTACACAATGCTAAGACTAAACCTCGCGAGGAGCAGTA |
| | | | CGCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1377 | ATX-P-3413 | HC IgG1 Fc nt | CAGGTGCAGCTGGTGCAGTCTGGGTCTGAGGTGAAGAAGCCT |
| | | | GGGGCCTCAGTGAAGGTCTCCTGCAAGACTTCTGGATACACGT |
| | | | TCACCGGCTACTATATGCACTGGGTGCGACAGGCCCCTGGACA |
| | | | AGGGCTTGAGTGGATGGGATGGATCAACCCTAACAGTGGTGG |
| | | | CACAACCTATGCACAGAAGTTTCAGGGCAGGGTCACCATGAC |
| | | | CAGGGACACGTCCATCAGCACAGCCTACATGGAGCTGAACAG |
| | | | GCTGAGATCTGACGACACGGCCGTGTATTACTGTGCGAGAGA |
| | | | GGCAGGTGGTTCAGGTGAATACTTCCAGCACTGGGGCCAGGG |
| | | | CACCCCGGTCACCGTCTCTTCAGCTAGCACTAAAGGGCCTTCT |
| | | | GTATTTCCCTTGGCCCCGTCCAGCAAATCGACCTCGGGAGGGA |
| | | | CAGCCGCCCTGGGTTGCCTTGTGAAAGATTATTTCCCTGAGCC |
| | | | AGTTACCGTAAGTTGGAACAGTGGGGCGCTGACAAGTGGTGT |
| | | | GCACACGTTTCCTGCCGTCCTGCAATCATCGGGCTTGTATAGC |
| | | | CTCAGCTCTGTGGTCACTGTCCCAAGTTCATCGCTGGGCACTC |
| | | | AGACGTATATTTGCAATGTGAACCACAAACCTTCAAATACAAA |
| | | | AGTGGATAAACGCGTAGAACCGAAATCGTGTGATAAAACTCA |
| | | | CACATGCCCGCCATGCCCGGCACCTGAACTGCTTGGTGGTCCC |
| | | | AGCGTGTTCCTGTTCCCGCCGAAGCCTAAAGATACTCTAATGA |
| | | | TCAGCCGTACGCCAGAGGTGACATGTGTCGTGGTTGACGTGTC |
| | | | CCACGAAGATCCCGAAGTTAAGTTCAATTGGTATGTTGATGGT |
| | | | GTAGAGGTACACAATGCTAAGACTAAACCTCGCGAGGAGCAG |
| | | | TACGCCTCGACCTATCGTGTCGTGAGCGTTCTGACCGTCCTTCA |
| | | | CCAAGATTGGCTTAACGGCAAAGAATATAAGTGCAAGGTAAG |
| | | | CAATAAAGCACTTCCGGCCCCAATCGAGAAAACCATTTCCAAG |
| | | | GCCAAAGGTCAACCAAGAGAACCCCAGGTGTATACTCTTCCGC |
| | | | CTTCTCGTGAGGAAATGACTAAAAATCAAGTATCCCTTACGTG |
| | | | TCTGGTTAAAGGTTTTTATCCTAGCGATATTGCTGTTGAATGGG |
| | | | AATCGAACGGTCAGCCGGAGAATAATTATAAAACAACGCCAC |
| | | | CCGTCCTGGATAGCGACGGCTCATTTTTTCTGTATAGCAAACT |
| | | | GACTGTAGATAAATCACGGTGGCAGCAGGGCAATGTATTCAG |
| | | | TTGCTCCGTTATGCATGAAGCGTTACATAATCACTACACGCAG |
| | | | AAATCTCTTAGTCTTTCACCCGGT |
| 1378 | ATX-P-3414 | HC IgG1 Fc nt | CAGATCACCTTGAAGGAGTCTGGTCCTACGCTGGTGAAACCCA |
| | | | CACAGACCCTCACGCTGACCTGCACCTTCTCTGGGTTCTCACTC |
| | | | AGCACTAGTGGAGTGGGTGTGGGCTGGATCCGTCAGCCCCCA |
| | | | GGAAAGGCCCTGGAGTGGCTTGCACTCATTTATTGGAATGATG |
| | | | TTAAGTTCTACAGTCCATCTCTGCAGAGCAGGCTCACCATCAC |
| | | | CAAGGACACCTCCAAAAACCAGGTGGTCCTTACACTGACCAA |
| | | | CATGGACCCTGTGGACACAGCCACATATTACTGTGCACACGGG |
| | | | GATAACTGGAACTACCGTGCTTTTGATATCTGGGGACAAGGGA |
| | | | CAATGGTCACCGTCTCTTCAGCTAGCACTAAAGGGCCTTCTGT |
| | | | ATTTCCCTTGGCCCCGTCCAGCAAATCGACCTCGGGAGGGACA |
| | | | GCCGCCCTGGGTTGCCTTGTGAAAGATTATTTCCCTGAGCCAG |
| | | | TTACCGTAAGTTGGAACAGTGGGGCGCTGACAAGTGGTGTGC |
| | | | ACACGTTTCCTGCCGTCCTGCAATCATCGGGCTTGTATAGCCTC |
| | | | AGCTCTGTGGTCACTGTCCCAAGTTCATCGCTGGGCACTCAGA |
| | | | CGTATATTTGCAATGTGAACCACAAACCTTCAAATACAAAAGT |
| | | | GGATAAACGCGTAGAACCGAAATCGTGTGATAAAACTCACAC |
| | | | ATGCCCGCCATGCCCGGCACCTGAACTGCTTGGTGGTCCCAGC |
| | | | GTGTTCCTGTTCCCGCCGAAGCCTAAAGATACTCTAATGATCA |
| | | | GCCGTACGCCAGAGGTGACATGTGTCGTGGTTGACGTGTCCCA |
| | | | CGAAGATCCCGAAGTTAAGTTCAATTGGTATGTTGATGGTGTA |
| | | | GAGGTACACAATGCTAAGACTAAACCTCGCGAGGAGCAGTAC |
| | | | GCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1379 | ATX-P-3419 | HC IgG1 Fc nt | CAGGTTCAGCTGGTGCAGTCTGGAGCTGAGGTGAAGAAGCCT |
| | | | GGGGCCTCAGTGAAGGTCTCCTGCAAGGCTTCTGGTTACACCT |
| | | | TTACCAGCTATGGTATCAGCTGGGTGCGACAGGCCCCTGGACA |
| | | | AGGGCTTGAGTGGATGGGATGGATCAGCGCTTATAATGGTAA |
| | | | CACACACTATGCACAGAACCTCCAGGGCAGAGTCACCATGAC |
| | | | CACAGACACATCCACGAGCACAGCCTACATGGAGCTGAGGAG |
| | | | CCTGAGATCTGACGACACGGCCGTGTATTACTGTGCGAGGAGT |
| | | | ATAGCAGCCCTTGACTTCTGGGGCCAGGGAACCCCGGTCACCG |
| | | | TCTCCTCAGCTAGCACTAAAGGGCCTTCTGTATTTCCCTTGGCC |
| | | | CCGTCCAGCAAATCGACCTCGGGAGGGACAGCCGCCCTGGGT |
| | | | TGCCTTGTGAAAGATTATTTCCCTGAGCCAGTTACCGTAAGTT |
| | | | GGAACAGTGGGGCGCTGACAAGTGGTGTGCACACGTTTCCTGC |
| | | | CGTCCTGCAATCATCGGGCTTGTATAGCCTCAGCTCTGTGGTC |
| | | | ACTGTCCCAAGTTCATCGCTGGGCACTCAGACGTATATTTGCA |
| | | | ATGTGAACCACAAACCTTCAAATACAAAAGTGGATAAACGCG |
| | | | TAGAACCGAAATCGTGTGATAAAACTCACACATGCCCGCCATG |
| | | | CCCGGCACCTGAACTGCTTGGTGGTCCCAGCGTGTTCCTGTTC |
| | | | CCGCCGAAGCCTAAAGATACTCTAATGATCAGCCGTACGCCAG |
| | | | AGGTGACATGTGTCGTGGTTGACGTGTCCCACGAAGATCCCGA |
| | | | AGTTAAGTTCAATTGGTATGTTGATGGTGTAGAGGTACACAAT |
| | | | GCTAAGACTAAACCTCGCGAGGAGCAGTACGCCTCGACCTATC |
| | | | GTGTCGTGAGCGTTCTGACCGTCCTTCACCAAGATTGGCTTAA |
| | | | CGGCAAAGAATATAAGTGCAAGGTAAGCAATAAAGCACTTCC |
| | | | GGCCCCAATCGAGAAAACCATTTCCAAGGCCAAAGGTCAACC |
| | | | AAGAGAACCCCAGGTGTATACTCTTCCGCCTTCTCGTGAGGAA |
| | | | ATGACTAAAAATCAAGTATCCCTTACGTGTCTGGTTAAAGGTT |
| | | | TTTATCCTAGCGATATTGCTGTTGAATGGGAATCGAACGGTCA |
| | | | GCCGGAGAATAATTATAAAACAACGCCACCCGTCCTGGATAG |
| | | | CGACGGCTCATTTTTTCTGTATAGCAAACTGACTGTAGATAAA |
| | | | TCACGGTGGCAGCAGGGCAATGTATTCAGTTGCTCCGTTATGC |
| | | | ATGAAGCGTTACATAATCACTACACGCAGAAATCTCTTAGTCT |
| | | | TTCACCCGGT |
| 1380 | ATX-P-3421 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCAGTAATAACTGGTGGAGTTGGGTCCGCCAGCCCCCAGG |
| | | | GAAGGGACTGGAGTGGATTGGACAAATCTATCATAGTGAAAA |
| | | | CACCAACTACAACCCGTCCCTCAAGAGTCGAGTCACCATATCA |
| | | | GTAGACAAGTCCAAGAACCAGTTCTCCCTGAAGCTGAGCTCTG |
| | | | TGACCGCCGCGGACACGGCCATCTATTACTGTGCGAGAGTGGA |
| | | | TGACTGGAACTACGGGGGTCACTTTGACTACTGGGGCCAGGG |
| | | | AACCCTGGTCACCGTCTCCTCAGCTAGCACTAAAGGGCCTTCT |
| | | | GTATTTCCCTTGGCCCCGTCCAGCAAATCGACCTCGGGAGGGA |
| | | | CAGCCGCCCTGGGTTGCCTTGTGAAAGATTATTTCCCTGAGCC |
| | | | AGTTACCGTAAGTTGGAACAGTGGGGCGCTGACAAGTGGTGT |
| | | | GCACACGTTTCCTGCCGTCCTGCAATCATCGGGCTTGTATAGC |
| | | | CTCAGCTCTGTGGTCACTGTCCCAAGTTCATCGCTGGGCACTC |
| | | | AGACGTATATTTGCAATGTGAACCACAAACCTTCAAATACAAA |
| | | | AGTGGATAAACGCGTAGAACCGAAATCGTGTGATAAAACTCA |
| | | | CACATGCCCGCCATGCCCGGCACCTGAACTGCTTGGTGGTCCC |
| | | | AGCGTGTTCCTGTTCCCGCCGAAGCCTAAAGATACTCTAATGA |
| | | | TCAGCCGTACGCCAGAGGTGACATGTGTCGTGGTTGACGTGTC |
| | | | CCACGAAGATCCCGAAGTTAAGTTCAATTGGTATGTTGATGGT |
| | | | GTAGAGGTACACAATGCTAAGACTAAACCTCGCGAGGAGCAG |
| | | | TACGCCTCGACCTATCGTGTCGTGAGCGTTCTGACCGTCCTTCA |
| | | | CCAAGATTGGCTTAACGGCAAAGAATATAAGTGCAAGGTAAG |
| | | | CAATAAAGCACTTCCGGCCCCAATCGAGAAAACCATTTCCAAG |
| | | | GCCAAAGGTCAACCAAGAGAACCCCAGGTGTATACTCTTCCGC |
| | | | CTTCTCGTGAGGAAATGACTAAAAATCAAGTATCCCTTACGTG |
| | | | TCTGGTTAAAGGTTTTTATCCTAGCGATATTGCTGTTGAATGGG |
| | | | AATCGAACGGTCAGCCGGAGAATAATTATAAAACAACGCCAC |
| | | | CCGTCCTGGATAGCGACGGCTCATTTTTTCTGTATAGCAAACT |
| | | | GACTGTAGATAAATCACGGTGGCAGCAGGGCAATGTATTCAG |
| | | | TTGCTCCGTTATGCATGAAGCGTTACATAATCACTACACGCAG |
| | | | AAATCTCTTAGTCTTTCACCCGGT |
| 1381 | ATX-P-3428 | HC IgG1 Fc nt | CAGGTTCAGCTGGTGCAGTCTGGAGCTGAGGTGAAGAAGCCT |
| | | | GGGGCCTCAGTTAAGGTCTCCTGCAAGGCTTCTGGTTACACCT |
| | | | TTACCGGCTATGGTATCAGCTGGGTGCGACAGGCCCCTGGACA |
| | | | AGGGCTTGAGTGGATGGGATGGATCAGCGCTTACAATGGTAA |
| | | | CACACACTATGCACAGAACCTCCAGGGCAGAGTCACCATGAC |
| | | | CACCGACACATCCACGAACACAGCCTACATGGAGCTGAGGAG |
| | | | CCTTATATCTGACGACACGGCCTTTTATTATTGTGCGAGAGCG |
| | | | GGACTGGGGATCGACTACTGGGGCCAGGGAACCCTGGTCACT |
| | | | GTCTCCTCAGCTAGCACTAAAGGGCCTTCTGTATTTCCCTTGGC |
| | | | CCCGTCCAGCAAATCGACCTCGGGAGGGACAGCCGCCCTGGG |
| | | | TTGCCTTGTGAAAGATTATTTCCCTGAGCCAGTTACCGTAAGTT |
| | | | GGAACAGTGGGGCGCTGACAAGTGGTGTGCACACGTTTCCTGC |
| | | | CGTCCTGCAATCATCGGGCTTGTATAGCCTCAGCTCTGTGGTC |
| | | | ACTGTCCCAAGTTCATCGCTGGGCACTCAGACGTATATTTGCA |
| | | | ATGTGAACCACAAACCTTCAAATACAAAAGTGGATAAACGCG |
| | | | TAGAACCGAAATCGTGTGATAAAACTCACACATGCCCGCCATG |
| | | | CCCGGCACCTGAACTGCTTGGTGGTCCCAGCGTGTTCCTGTTC |
| | | | CCGCCGAAGCCTAAAGATACTCTAATGATCAGCCGTACGCCAG |
| | | | AGGTGACATGTGTCGTGGTTGACGTGTCCCACGAAGATCCCGA |
| | | | AGTTAAGTTCAATTGGTATGTTGATGGTGTAGAGGTACACAAT |
| | | | GCTAAGACTAAACCTCGCGAGGAGCAGTACGCCTCGACCTATC |
| | | | GTGTCGTGAGCGTTCTGACCGTCCTTCACCAAGATTGGCTTAA |
| | | | CGGCAAAGAATATAAGTGCAAGGTAAGCAATAAAGCACTTCC |
| | | | GGCCCCAATCGAGAAAACCATTTCCAAGGCCAAAGGTCAACC |
| | | | AAGAGAACCCCAGGTGTATACTCTTCCGCCTTCTCGTGAGGAA |
| | | | ATGACTAAAAATCAAGTATCCCTTACGTGTCTGGTTAAAGGTT |
| | | | TTTATCCTAGCGATATTGCTGTTGAATGGGAATCGAACGGTCA |
| | | | GCCGGAGAATAATTATAAAACAACGCCACCCGTCCTGGATAG |
| | | | CGACGGCTCATTTTTTCTGTATAGCAAACTGACTGTAGATAAA |
| | | | TCACGGTGGCAGCAGGGCAATGTATTCAGTTGCTCCGTTATGC |
| | | | ATGAAGCGTTACATAATCACTACACGCAGAAATCTCTTAGTCT |
| | | | TTCACCCGGT |
| 1382 | ATX-P-3430 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCAGTAGTGACTGGTGGAGTTGGGTCCGCCAGTCCCCAGG |
| | | | GAAGGGGCTGGAGTGGATTGGGGAAATCTATCATAGTGGGAG |
| | | | CACCAACTACAACCCGTCCCTCAAGAGTCGAGTCTCCATATCA |
| | | | GTAGACAAGTCCAAGAACCAGTTCTCCCTGAAGCTGAGCTCTG |
| | | | TGACCGCCGCGGACACGGCCGTGTATTACTGTGCGAGAGGGG |
| | | | GGCACATAGGCTACTACTACATGGACGTCTGGGGCAAAGGGA |
| | | | CCACGGTCACCGTCTCCTCAGCTAGCACTAAAGGGCCTTCTGT |
| | | | ATTTCCCTTGGCCCCGTCCAGCAAATCGACCTCGGGAGGGACA |
| | | | GCCGCCCTGGGTTGCCTTGTGAAAGATTATTTCCCTGAGCCAG |
| | | | TTACCGTAAGTTGGAACAGTGGGGCGCTGACAAGTGGTGTGC |
| | | | ACACGTTTCCTGCCGTCCTGCAATCATCGGGCTTGTATAGCCTC |
| | | | AGCTCTGTGGTCACTGTCCCAAGTTCATCGCTGGGCACTCAGA |
| | | | CGTATATTTGCAATGTGAACCACAAACCTTCAAATACAAAAGT |
| | | | GGATAAACGCGTAGAACCGAAATCGTGTGATAAAACTCACAC |
| | | | ATGCCCGCCATGCCCGGCACCTGAACTGCTTGGTGGTCCCAGC |
| | | | GTGTTCCTGTTCCCGCCGAAGCCTAAAGATACTCTAATGATCA |
| | | | GCCGTACGCCAGAGGTGACATGTGTCGTGGTTGACGTGTCCCA |
| | | | CGAAGATCCCGAAGTTAAGTTCAATTGGTATGTTGATGGTGTA |
| | | | GAGGTACACAATGCTAAGACTAAACCTCGCGAGGAGCAGTAC |
| | | | GCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1383 | ATX-P-3431 | HC IgG1 Fc nt | GAGGTGCAGCTGGTGCAGTCTGGAGCAGAGGTGAAAAAGCCC |
| | | | GGGGAGTCTCTGAAGATCTCCTGTAAGGGTTCTGGATACAGCT |
| | | | TTACCAACTACTGGATCGGCTGGGTGCGCCAGATGCCCGGGAA |
| | | | AGGCCTGGAGTGGATGGGGATCATCTATCCTGGTGACTCTGAT |
| | | | ACCATATACAGCCCGTCCTTCCAAGGCCAGGTCACCTTCTCAG |
| | | | CCGACAAGTCCATCAACACCGCCTACCTGCAGTGGAGCAGCCT |
| | | | GAAGGCCTCGGACACCGCCATGTATTACTGTGCGAGAGGACG |
| | | | TGGATATAGTGGCTACGAAGGTGCTTTTGATATCTGGGGACAA |
| | | | GGGACAATGGTCACCGTCTCTTCAGCTAGCACTAAAGGGCCTT |
| | | | CTGTATTTCCCTTGGCCCCGTCCAGCAAATCGACCTCGGGAGG |
| | | | GACAGCCGCCCTGGGTTGCCTTGTGAAAGATTATTTCCCTGAG |
| | | | CCAGTTACCGTAAGTTGGAACAGTGGGGCGCTGACAAGTGGT |
| | | | GTGCACACGTTTCCTGCCGTCCTGCAATCATCGGGCTTGTATA |
| | | | GCCTCAGCTCTGTGGTCACTGTCCCAAGTTCATCGCTGGGCAC |
| | | | TCAGACGTATATTTGCAATGTGAACCACAAACCTTCAAATACA |
| | | | AAAGTGGATAAACGCGTAGAACCGAAATCGTGTGATAAAACT |
| | | | CACACATGCCCGCCATGCCCGGCACCTGAACTGCTTGGTGGTC |
| | | | CCAGCGTGTTCCTGTTCCCGCCGAAGCCTAAAGATACTCTAAT |
| | | | GATCAGCCGTACGCCAGAGGTGACATGTGTCGTGGTTGACGTG |
| | | | TCCCACGAAGATCCCGAAGTTAAGTTCAATTGGTATGTTGATG |
| | | | GTGTAGAGGTACACAATGCTAAGACTAAACCTCGCGAGGAGC |
| | | | AGTACGCCTCGACCTATCGTGTCGTGAGCGTTCTGACCGTCCT |
| | | | TCACCAAGATTGGCTTAACGGCAAAGAATATAAGTGCAAGGT |
| | | | AAGCAATAAAGCACTTCCGGCCCCAATCGAGAAAACCATTTCC |
| | | | AAGGCCAAAGGTCAACCAAGAGAACCCCAGGTGTATACTCTT |
| | | | CCGCCTTCTCGTGAGGAAATGACTAAAAATCAAGTATCCCTTA |
| | | | CGTGTCTGGTTAAAGGTTTTTATCCTAGCGATATTGCTGTTGAA |
| | | | TGGGAATCGAACGGTCAGCCGGAGAATAATTATAAAACAACG |
| | | | CCACCCGTCCTGGATAGCGACGGCTCATTTTTTCTGTATAGCA |
| | | | AACTGACTGTAGATAAATCACGGTGGCAGCAGGGCAATGTAT |
| | | | TCAGTTGCTCCGTTATGCATGAAGCGTTACATAATCACTACAC |
| | | | GCAGAAATCTCTTAGTCTTTCACCCGGT |
| 1384 | ATX-P-3435 | HC IgG1 Fc nt | CAGGTTCAGCTGGTGCAGTCTGGAGCTGAGGTGAAGAAGCCT |
| | | | GGGGCCTCAGTGAAGGTCTCCTGCAAGGCTTCTGGTTACACCT |
| | | | TTACCAACTATGGTATCAGCTGGGTGCGACAGGCCCCTGGACA |
| | | | AGGGCTTGAGTGGATGGGATGGATCAGCGCTTACAATGGTAA |
| | | | CACACACTATGCACAGAAACTCCAGGGCAGAGTCACCATGAC |
| | | | CACAGACACATCCACGAGCACAGCCTACATGGAGCTGAGGAG |
| | | | CCTGAGATCAGACGACACGGCCGTGTATTACTGTGCGAGAGC |
| | | | GGCAGCAGCCGTTGACTACTGGGGCCAGGGAACCCTGGTCAC |
| | | | CGTCTCTTCAGCTAGCACTAAAGGGCCTTCTGTATTTCCCTTGG |
| | | | CCCCGTCCAGCAAATCGACCTCGGGAGGGACAGCCGCCCTGG |
| | | | GTTGCCTTGTGAAAGATTATTTCCCTGAGCCAGTTACCGTAAG |
| | | | TTGGAACAGTGGGGCGCTGACAAGTGGTGTGCACACGTTTCCT |
| | | | GCCGTCCTGCAATCATCGGGCTTGTATAGCCTCAGCTCTGTGG |
| | | | TCACTGTCCCAAGTTCATCGCTGGGCACTCAGACGTATATTTG |
| | | | CAATGTGAACCACAAACCTTCAAATACAAAAGTGGATAAACG |
| | | | CGTAGAACCGAAATCGTGTGATAAAACTCACACATGCCCGCC |
| | | | ATGCCCGGCACCTGAACTGCTTGGTGGTCCCAGCGTGTTCCTG |
| | | | TTCCCGCCGAAGCCTAAAGATACTCTAATGATCAGCCGTACGC |
| | | | CAGAGGTGACATGTGTCGTGGTTGACGTGTCCCACGAAGATCC |
| | | | CGAAGTTAAGTTCAATTGGTATGTTGATGGTGTAGAGGTACAC |
| | | | AATGCTAAGACTAAACCTCGCGAGGAGCAGTACGCCTCGACC |
| | | | TATCGTGTCGTGAGCGTTCTGACCGTCCTTCACCAAGATTGGC |
| | | | TTAACGGCAAAGAATATAAGTGCAAGGTAAGCAATAAAGCAC |
| | | | TTCCGGCCCCAATCGAGAAAACCATTTCCAAGGCCAAAGGTCA |
| | | | ACCAAGAGAACCCCAGGTGTATACTCTTCCGCCTTCTCGTGAG |
| | | | GAAATGACTAAAAATCAAGTATCCCTTACGTGTCTGGTTAAAG |
| | | | GTTTTTATCCTAGCGATATTGCTGTTGAATGGGAATCGAACGG |
| | | | TCAGCCGGAGAATAATTATAAAACAACGCCACCCGTCCTGGAT |
| | | | AGCGACGGCTCATTTTTTCTGTATAGCAAACTGACTGTAGATA |
| | | | AATCACGGTGGCAGCAGGGCAATGTATTCAGTTGCTCCGTTAT |
| | | | GCATGAAGCGTTACATAATCACTACACGCAGAAATCTCTTAGT |
| | | | CTTTCACCCGGT |
| 1385 | ATX-P-3436 | HC IgG1 Fc nt | CAGGTGCAGCTGCAGGAGTCGGGTCCAGGACTGGTGAAGCCT |
| | | | TCGGAGACCCTGTCCCTCAGTTGCACTGTCTCTGGTGGCTCCGT |
| | | | CAGCAGTGGTAGTTACTACTGGAGCTGGATCCGGCAGCCCCCA |
| | | | GGGAAGGGACTGGAGTGGATTGGGTATATCTATTACAGTGGG |
| | | | AGCACCAAGTACAACCCCTCCCTCAAGAGTCGAGTCACCATAT |
| | | | CAGTAGACACGTCCAAGAACCAGGTCTCCCTGAAGCTGAGTTC |
| | | | TGTGACCGCTGCGGACACGGCCGTGTATTACTGTGCGAGAGAT |
| | | | AAGGGGCACTGGAACTACGGTGCTTTTGATATCTGGGGACAA |
| | | | GGGACAATGGTCACCGTCTCTTCAGCTAGCACTAAAGGGCCTT |
| | | | CTGTATTTCCCTTGGCCCCGTCCAGCAAATCGACCTCGGGAGG |
| | | | GACAGCCGCCCTGGGTTGCCTTGTGAAAGATTATTTCCCTGAG |
| | | | CCAGTTACCGTAAGTTGGAACAGTGGGGCGCTGACAAGTGGT |
| | | | GTGCACACGTTTCCTGCCGTCCTGCAATCATCGGGCTTGTATA |
| | | | GCCTCAGCTCTGTGGTCACTGTCCCAAGTTCATCGCTGGGCAC |
| | | | TCAGACGTATATTTGCAATGTGAACCACAAACCTTCAAATACA |
| | | | AAAGTGGATAAACGCGTAGAACCGAAATCGTGTGATAAAACT |
| | | | CACACATGCCCGCCATGCCCGGCACCTGAACTGCTTGGTGGTC |
| | | | CCAGCGTGTTCCTGTTCCCGCCGAAGCCTAAAGATACTCTAAT |
| | | | GATCAGCCGTACGCCAGAGGTGACATGTGTCGTGGTTGACGTG |
| | | | TCCCACGAAGATCCCGAAGTTAAGTTCAATTGGTATGTTGATG |
| | | | GTGTAGAGGTACACAATGCTAAGACTAAACCTCGCGAGGAGC |
| | | | AGTACGCCTCGACCTATCGTGTCGTGAGCGTTCTGACCGTCCT |
| | | | TCACCAAGATTGGCTTAACGGCAAAGAATATAAGTGCAAGGT |
| | | | AAGCAATAAAGCACTTCCGGCCCCAATCGAGAAAACCATTTCC |
| | | | AAGGCCAAAGGTCAACCAAGAGAACCCCAGGTGTATACTCTT |
| | | | CCGCCTTCTCGTGAGGAAATGACTAAAAATCAAGTATCCCTTA |
| | | | CGTGTCTGGTTAAAGGTTTTTATCCTAGCGATATTGCTGTTGAA |
| | | | TGGGAATCGAACGGTCAGCCGGAGAATAATTATAAAACAACG |
| | | | CCACCCGTCCTGGATAGCGACGGCTCATTTTTTCTGTATAGCA |
| | | | AACTGACTGTAGATAAATCACGGTGGCAGCAGGGCAATGTAT |
| | | | TCAGTTGCTCCGTTATGCATGAAGCGTTACATAATCACTACAC |
| | | | GCAGAAATCTCTTAGTCTTTCACCCGGT |
| 1386 | ATX-P-3437 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCAGTAATAACTGGTGGAGTTGGGTCCGCCAGCCCCCAGG |
| | | | GAAGGGGCTGGAGTGGATTGGGGAAATCTATCATAGTGGAAA |
| | | | TATTAACTACAACCCGTCCCTCAAGAGTCGAGTCACCATATCA |
| | | | GTGGACAAGTCCAAGAACCAGTTCTCCCTGAAGCTGAGCTCTG |
| | | | TGACCGCCGCGGACACGGCCGTGTATTACTGTGCGAGAGGGG |
| | | | GGGAATACGGTGACCCCGTGGGGTACTTCGATCTCTGGGGCCG |
| | | | TGGCACCCTGGTCACTGTCTCCTCAGCTAGCACTAAAGGGCCT |
| | | | TCTGTATTTCCCTTGGCCCCGTCCAGCAAATCGACCTCGGGAG |
| | | | GGACAGCCGCCCTGGGTTGCCTTGTGAAAGATTATTTCCCTGA |
| | | | GCCAGTTACCGTAAGTTGGAACAGTGGGGCGCTGACAAGTGG |
| | | | TGTGCACACGTTTCCTGCCGTCCTGCAATCATCGGGCTTGTATA |
| | | | GCCTCAGCTCTGTGGTCACTGTCCCAAGTTCATCGCTGGGCAC |
| | | | TCAGACGTATATTTGCAATGTGAACCACAAACCTTCAAATACA |
| | | | AAAGTGGATAAACGCGTAGAACCGAAATCGTGTGATAAAACT |
| | | | CACACATGCCCGCCATGCCCGGCACCTGAACTGCTTGGTGGTC |
| | | | CCAGCGTGTTCCTGTTCCCGCCGAAGCCTAAAGATACTCTAAT |
| | | | GATCAGCCGTACGCCAGAGGTGACATGTGTCGTGGTTGACGTG |
| | | | TCCCACGAAGATCCCGAAGTTAAGTTCAATTGGTATGTTGATG |
| | | | GTGTAGAGGTACACAATGCTAAGACTAAACCTCGCGAGGAGC |
| | | | AGTACGCCTCGACCTATCGTGTCGTGAGCGTTCTGACCGTCCT |
| | | | TCACCAAGATTGGCTTAACGGCAAAGAATATAAGTGCAAGGT |
| | | | AAGCAATAAAGCACTTCCGGCCCCAATCGAGAAAACCATTTCC |
| | | | AAGGCCAAAGGTCAACCAAGAGAACCCCAGGTGTATACTCTT |
| | | | CCGCCTTCTCGTGAGGAAATGACTAAAAATCAAGTATCCCTTA |
| | | | CGTGTCTGGTTAAAGGTTTTTATCCTAGCGATATTGCTGTTGAA |
| | | | TGGGAATCGAACGGTCAGCCGGAGAATAATTATAAAACAACG |
| | | | CCACCCGTCCTGGATAGCGACGGCTCATTTTTTCTGTATAGCA |
| | | | AACTGACTGTAGATAAATCACGGTGGCAGCAGGGCAATGTAT |
| | | | TCAGTTGCTCCGTTATGCATGAAGCGTTACATAATCACTACAC |
| | | | GCAGAAATCTCTTAGTCTTTCACCCGGT |
| 1387 | ATX-P-3440 | HC IgG1 Fc nt | CAGGTTCAGCTGGTGCAGTCTGGAGCTGAGGTGAAGAAGCCT |
| | | | GGGGCCTCAGTGAAGGTCTCCTGCAAGGCTTCTGGTTACACCT |
| | | | TTACCACCTATGGTATCAGCTGGGTGCGACAGGCCCCTGGACA |
| | | | AGGGCTTGAGTGGATGGGATGGATCAGCGCTTACAGTGGTAA |
| | | | CACAAACTATGCACAGAAGCTCCAGGGCAGAGTCACCATGAC |
| | | | CACAGACACATCCACGAGCACAGCCTACATGGAGCTGAGGAG |
| | | | CCTGAGATCTGACGACACGGCCGTGTATTACTGTGCGAGAGAG |
| | | | AGAGTCTCCAACTGGGTCTTTGACTTCTGGGGCCAGGGAACCC |
| | | | TGGTCACTGTCTCCTCAGCTAGCACTAAAGGGCCTTCTGTATTT |
| | | | CCCTTGGCCCCGTCCAGCAAATCGACCTCGGGAGGGACAGCC |
| | | | GCCCTGGGTTGCCTTGTGAAAGATTATTTCCCTGAGCCAGTTA |
| | | | CCGTAAGTTGGAACAGTGGGGCGCTGACAAGTGGTGTGCACA |
| | | | CGTTTCCTGCCGTCCTGCAATCATCGGGCTTGTATAGCCTCAGC |
| | | | TCTGTGGTCACTGTCCCAAGTTCATCGCTGGGCACTCAGACGT |
| | | | ATATTTGCAATGTGAACCACAAACCTTCAAATACAAAAGTGGA |
| | | | TAAACGCGTAGAACCGAAATCGTGTGATAAAACTCACACATG |
| | | | CCCGCCATGCCCGGCACCTGAACTGCTTGGTGGTCCCAGCGTG |
| | | | TTCCTGTTCCCGCCGAAGCCTAAAGATACTCTAATGATCAGCC |
| | | | GTACGCCAGAGGTGACATGTGTCGTGGTTGACGTGTCCCACGA |
| | | | AGATCCCGAAGTTAAGTTCAATTGGTATGTTGATGGTGTAGAG |
| | | | GTACACAATGCTAAGACTAAACCTCGCGAGGAGCAGTACGCC |
| | | | TCGACCTATCGTGTCGTGAGCGTTCTGACCGTCCTTCACCAAG |
| | | | ATTGGCTTAACGGCAAAGAATATAAGTGCAAGGTAAGCAATA |
| | | | AAGCACTTCCGGCCCCAATCGAGAAAACCATTTCCAAGGCCA |
| | | | AAGGTCAACCAAGAGAACCCCAGGTGTATACTCTTCCGCCTTC |
| | | | TCGTGAGGAAATGACTAAAAATCAAGTATCCCTTACGTGTCTG |
| | | | GTTAAAGGTTTTTATCCTAGCGATATTGCTGTTGAATGGGAAT |
| | | | CGAACGGTCAGCCGGAGAATAATTATAAAACAACGCCACCCG |
| | | | TCCTGGATAGCGACGGCTCATTTTTTCTGTATAGCAAACTGAC |
| | | | TGTAGATAAATCACGGTGGCAGCAGGGCAATGTATTCAGTTGC |
| | | | TCCGTTATGCATGAAGCGTTACATAATCACTACACGCAGAAAT |
| | | | CTCTTAGTCTTTCACCCGGT |
| 1388 | ATX-P-3442 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGTTCCAT |
| | | | CAGCAGTAGTGACTGGTGGAGCTGGGTCCGCCAGCCCCCAGG |
| | | | GAAGGGGCTGGAGTGGATTGGGGAAATCTATCATAGTAGGAA |
| | | | CACCAACTACAACCCGTCCCTCAAGAGTCGAGTCACCATATCA |
| | | | GTAGACAAGTCCAAGAACCAGTTCTCCCTGAAGCTGAGTTCTG |
| | | | TGACCGCCGCGGACACGGCCGTGTATTACTGTGCGAGTGGAG |
| | | | GTGTGGTGGCCGACTACTACATGGACGTCTGGGGCAAAGGGA |
| | | | CCACGGTCACCGTCTCCTCAGCTAGCACTAAAGGGCCTTCTGT |
| | | | ATTTCCCTTGGCCCCGTCCAGCAAATCGACCTCGGGAGGGACA |
| | | | GCCGCCCTGGGTTGCCTTGTGAAAGATTATTTCCCTGAGCCAG |
| | | | TTACCGTAAGTTGGAACAGTGGGGCGCTGACAAGTGGTGTGC |
| | | | ACACGTTTCCTGCCGTCCTGCAATCATCGGGCTTGTATAGCCTC |
| | | | AGCTCTGTGGTCACTGTCCCAAGTTCATCGCTGGGCACTCAGA |
| | | | CGTATATTTGCAATGTGAACCACAAACCTTCAAATACAAAAGT |
| | | | GGATAAACGCGTAGAACCGAAATCGTGTGATAAAACTCACAC |
| | | | ATGCCCGCCATGCCCGGCACCTGAACTGCTTGGTGGTCCCAGC |
| | | | GTGTTCCTGTTCCCGCCGAAGCCTAAAGATACTCTAATGATCA |
| | | | GCCGTACGCCAGAGGTGACATGTGTCGTGGTTGACGTGTCCCA |
| | | | CGAAGATCCCGAAGTTAAGTTCAATTGGTATGTTGATGGTGTA |
| | | | GAGGTACACAATGCTAAGACTAAACCTCGCGAGGAGCAGTAC |
| | | | GCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1389 | ATX-P-3444 | HC IgG1 Fc nt | GAGGTGCAGCTGGTGGAGTCTGGGGGAGGCCTGGTCAAGCCT |
| | | | GGGGGGTCCCTGAGACTCTCCTGTGCAGCCTCTGGATTCACCT |
| | | | TCAGTAGCTATGGCATGAACTGGGTCCGCCAGGCTCCAGGGA |
| | | | AGGGGCTGGAGTGGGTCTCATCCATTAGTGGTTATGGTAGTTA |
| | | | CATATACTACGCAGCCTCAGTGAAGGGCCGATTCACCATCTCC |
| | | | AGAGACAGCGCCAAGAACTCACTGTATCTGCAAATGAACAGC |
| | | | CTGAGCGCCGAGGACACAGCTGTGTATTACTGTGCGAGAGAA |
| | | | TTAGGTGGGATCTACTACGGGGTAGGGTACTTCGATCTCTGGG |
| | | | GCCGTGGCACCCTGGTCACTGTCTCCTCAGCTAGCACTAAAGG |
| | | | GCCTTCTGTATTTCCCTTGGCCCCGTCCAGCAAATCGACCTCGG |
| | | | GAGGGACAGCCGCCCTGGGTTGCCTTGTGAAAGATTATTTCCC |
| | | | TGAGCCAGTTACCGTAAGTTGGAACAGTGGGGCGCTGACAAG |
| | | | TGGTGTGCACACGTTTCCTGCCGTCCTGCAATCATCGGGCTTGT |
| | | | ATAGCCTCAGCTCTGTGGTCACTGTCCCAAGTTCATCGCTGGG |
| | | | CACTCAGACGTATATTTGCAATGTGAACCACAAACCTTCAAAT |
| | | | ACAAAAGTGGATAAACGCGTAGAACCGAAATCGTGTGATAAA |
| | | | ACTCACACATGCCCGCCATGCCCGGCACCTGAACTGCTTGGTG |
| | | | GTCCCAGCGTGTTCCTGTTCCCGCCGAAGCCTAAAGATACTCT |
| | | | AATGATCAGCCGTACGCCAGAGGTGACATGTGTCGTGGTTGAC |
| | | | GTGTCCCACGAAGATCCCGAAGTTAAGTTCAATTGGTATGTTG |
| | | | ATGGTGTAGAGGTACACAATGCTAAGACTAAACCTCGCGAGG |
| | | | AGCAGTACGCCTCGACCTATCGTGTCGTGAGCGTTCTGACCGT |
| | | | CCTTCACCAAGATTGGCTTAACGGCAAAGAATATAAGTGCAA |
| | | | GGTAAGCAATAAAGCACTTCCGGCCCCAATCGAGAAAACCAT |
| | | | TTCCAAGGCCAAAGGTCAACCAAGAGAACCCCAGGTGTATAC |
| | | | TCTTCCGCCTTCTCGTGAGGAAATGACTAAAAATCAAGTATCC |
| | | | CTTACGTGTCTGGTTAAAGGTTTTTATCCTAGCGATATTGCTGT |
| | | | TGAATGGGAATCGAACGGTCAGCCGGAGAATAATTATAAAAC |
| | | | AACGCCACCCGTCCTGGATAGCGACGGCTCATTTTTTCTGTAT |
| | | | AGCAAACTGACTGTAGATAAATCACGGTGGCAGCAGGGCAAT |
| | | | GTATTCAGTTGCTCCGTTATGCATGAAGCGTTACATAATCACT |
| | | | ACACGCAGAAATCTCTTAGTCTTTCACCCGGT |
| 1390 | ATX-P-3447 | HC IgG1 Fc nt | CAGGTGCAGCTGCAGGAGTCGGGCCCAGGACTGGTGAAGCCT |
| | | | TCGGAGACCCTGTCCCTCACCTGCGCTGTCTCTGGTGGCTCCGT |
| | | | CAGCAGTGGTAATTACTATTGGAGCTGGATCCGGCAGCCCCCA |
| | | | GGGAAGGGACTGGAGTGGATTGGGTATATCTATTCCAGTGGG |
| | | | AGCACCAACTACAACCCCTCCCTCAAGAGTCGAGTCACCATAT |
| | | | CAATAGACACGTCCAAGAACCAGTTCTCCCTGAAGCTGAGCTC |
| | | | TGTGACCGCTGCGGACACGGCCGTGTATTACTGTGCGAGAAGT |
| | | | CCTCGTGGATACAGCTATGGTCCCTTTGCCTACTGGGGCCAGG |
| | | | GGACCCTGGTCACCGTCTCTTCAGCTAGCACTAAAGGGCCTTC |
| | | | TGTATTTCCCTTGGCCCCGTCCAGCAAATCGACCTCGGGAGGG |
| | | | ACAGCCGCCCTGGGTTGCCTTGTGAAAGATTATTTCCCTGAGC |
| | | | CAGTTACCGTAAGTTGGAACAGTGGGGCGCTGACAAGTGGTG |
| | | | TGCACACGTTTCCTGCCGTCCTGCAATCATCGGGCTTGTATAG |
| | | | CCTCAGCTCTGTGGTCACTGTCCCAAGTTCATCGCTGGGCACT |
| | | | CAGACGTATATTTGCAATGTGAACCACAAACCTTCAAATACAA |
| | | | AAGTGGATAAACGCGTAGAACCGAAATCGTGTGATAAAACTC |
| | | | ACACATGCCCGCCATGCCCGGCACCTGAACTGCTTGGTGGTCC |
| | | | CAGCGTGTTCCTGTTCCCGCCGAAGCCTAAAGATACTCTAATG |
| | | | ATCAGCCGTACGCCAGAGGTGACATGTGTCGTGGTTGACGTGT |
| | | | CCCACGAAGATCCCGAAGTTAAGTTCAATTGGTATGTTGATGG |
| | | | TGTAGAGGTACACAATGCTAAGACTAAACCTCGCGAGGAGCA |
| | | | GTACGCCTCGACCTATCGTGTCGTGAGCGTTCTGACCGTCCTTC |
| | | | ACCAAGATTGGCTTAACGGCAAAGAATATAAGTGCAAGGTAA |
| | | | GCAATAAAGCACTTCCGGCCCCAATCGAGAAAACCATTTCCAA |
| | | | GGCCAAAGGTCAACCAAGAGAACCCCAGGTGTATACTCTTCC |
| | | | GCCTTCTCGTGAGGAAATGACTAAAAATCAAGTATCCCTTACG |
| | | | TGTCTGGTTAAAGGTTTTTATCCTAGCGATATTGCTGTTGAATG |
| | | | GGAATCGAACGGTCAGCCGGAGAATAATTATAAAACAACGCC |
| | | | ACCCGTCCTGGATAGCGACGGCTCATTTTTTCTGTATAGCAAA |
| | | | CTGACTGTAGATAAATCACGGTGGCAGCAGGGCAATGTATTCA |
| | | | GTTGCTCCGTTATGCATGAAGCGTTACATAATCACTACACGCA |
| | | | GAAATCTCTTAGTCTTTCACCCGGT |
| 1391 | ATX-P-3448 | HC IgG1 Fc nt | CAGGTGCAGCTGCAGGAGTCGGGCCCAGGACTGGTGAAGCCT |
| | | | TCACAGACCCTGTCCCTCACCTGCACTGTCTCTGGTGGCTCCAT |
| | | | CAGCAGTGGTGGTTACAACTGGAACTGGATCCGCCAGCACCC |
| | | | AGGGAAGGGCCTGGAATGGATTGGGTACATCAATTACAGTGG |
| | | | GAATACCTATTACAACCCGTCCCTCAAGAGTCGAGTTACCATA |
| | | | TCAGTAGACACGTCTAAGAACCAGTTCTCCCTGAAGCTGAGTT |
| | | | CTGTGACTGCCGCGGACACGGCCGTTTATTACTGTGCGAGAGG |
| | | | GCCCCTGGATACAGCTATGGCCCCCTTTGACTACTGGGGCCAG |
| | | | GGAACCCTGGTCACCGTCTCCTCAGCTAGCACTAAAGGGCCTT |
| | | | CTGTATTTCCCTTGGCCCCGTCCAGCAAATCGACCTCGGGAGG |
| | | | GACAGCCGCCCTGGGTTGCCTTGTGAAAGATTATTTCCCTGAG |
| | | | CCAGTTACCGTAAGTTGGAACAGTGGGGCGCTGACAAGTGGT |
| | | | GTGCACACGTTTCCTGCCGTCCTGCAATCATCGGGCTTGTATA |
| | | | GCCTCAGCTCTGTGGTCACTGTCCCAAGTTCATCGCTGGGCAC |
| | | | TCAGACGTATATTTGCAATGTGAACCACAAACCTTCAAATACA |
| | | | AAAGTGGATAAACGCGTAGAACCGAAATCGTGTGATAAAACT |
| | | | CACACATGCCCGCCATGCCCGGCACCTGAACTGCTTGGTGGTC |
| | | | CCAGCGTGTTCCTGTTCCCGCCGAAGCCTAAAGATACTCTAAT |
| | | | GATCAGCCGTACGCCAGAGGTGACATGTGTCGTGGTTGACGTG |
| | | | TCCCACGAAGATCCCGAAGTTAAGTTCAATTGGTATGTTGATG |
| | | | GTGTAGAGGTACACAATGCTAAGACTAAACCTCGCGAGGAGC |
| | | | AGTACGCCTCGACCTATCGTGTCGTGAGCGTTCTGACCGTCCT |
| | | | TCACCAAGATTGGCTTAACGGCAAAGAATATAAGTGCAAGGT |
| | | | AAGCAATAAAGCACTTCCGGCCCCAATCGAGAAAACCATTTCC |
| | | | AAGGCCAAAGGTCAACCAAGAGAACCCCAGGTGTATACTCTT |
| | | | CCGCCTTCTCGTGAGGAAATGACTAAAAATCAAGTATCCCTTA |
| | | | CGTGTCTGGTTAAAGGTTTTTATCCTAGCGATATTGCTGTTGAA |
| | | | TGGGAATCGAACGGTCAGCCGGAGAATAATTATAAAACAACG |
| | | | CCACCCGTCCTGGATAGCGACGGCTCATTTTTTCTGTATAGCA |
| | | | AACTGACTGTAGATAAATCACGGTGGCAGCAGGGCAATGTAT |
| | | | TCAGTTGCTCCGTTATGCATGAAGCGTTACATAATCACTACAC |
| | | | GCAGAAATCTCTTAGTCTTTCACCCGGT |
| 1392 | ATX-P-3449 | HC IgG1 Fc nt | CAGGTGCAGCTGCAGGAGTCGGGCCCAGGACTGGTGAAGCCT |
| | | | TCGGAGACCCTGTCCCTCACCTGTACTGTCTCTGGTGACTCCAT |
| | | | CAGTAGTTACTACTGGAGTTGGATCCGGCAGCCCCCAGGGAA |
| | | | GGGACTGGAGTGGATTGGATATATCTATTATAGTGGGTTCACC |
| | | | AAGTACAACCCCTCCCTCAAGAGTCGAGTCACCATTTCAGTAG |
| | | | ACACGTCCAAGAACCAGTTCTCCCTGAGGCTGAGCTCTGTGAC |
| | | | CGCTGCGGACACGGCCGTGTATTACTGTGCGAGCGTCGATTCA |
| | | | AAGTGGAACTACGGGTCTTTTCATATCTGGGGACAAGGGACA |
| | | | ATGGTCACCGTCTCTTCAGCTAGCACTAAAGGGCCTTCTGTAT |
| | | | TTCCCTTGGCCCCGTCCAGCAAATCGACCTCGGGAGGGACAGC |
| | | | CGCCCTGGGTTGCCTTGTGAAAGATTATTTCCCTGAGCCAGTT |
| | | | ACCGTAAGTTGGAACAGTGGGGCGCTGACAAGTGGTGTGCAC |
| | | | ACGTTTCCTGCCGTCCTGCAATCATCGGGCTTGTATAGCCTCA |
| | | | GCTCTGTGGTCACTGTCCCAAGTTCATCGCTGGGCACTCAGAC |
| | | | GTATATTTGCAATGTGAACCACAAACCTTCAAATACAAAAGTG |
| | | | GATAAACGCGTAGAACCGAAATCGTGTGATAAAACTCACACA |
| | | | TGCCCGCCATGCCCGGCACCTGAACTGCTTGGTGGTCCCAGCG |
| | | | TGTTCCTGTTCCCGCCGAAGCCTAAAGATACTCTAATGATCAG |
| | | | CCGTACGCCAGAGGTGACATGTGTCGTGGTTGACGTGTCCCAC |
| | | | GAAGATCCCGAAGTTAAGTTCAATTGGTATGTTGATGGTGTAG |
| | | | AGGTACACAATGCTAAGACTAAACCTCGCGAGGAGCAGTACG |
| | | | CCTCGACCTATCGTGTCGTGAGCGTTCTGACCGTCCTTCACCA |
| | | | AGATTGGCTTAACGGCAAAGAATATAAGTGCAAGGTAAGCAA |
| | | | TAAAGCACTTCCGGCCCCAATCGAGAAAACCATTTCCAAGGCC |
| | | | AAAGGTCAACCAAGAGAACCCCAGGTGTATACTCTTCCGCCTT |
| | | | CTCGTGAGGAAATGACTAAAAATCAAGTATCCCTTACGTGTCT |
| | | | GGTTAAAGGTTTTTATCCTAGCGATATTGCTGTTGAATGGGAA |
| | | | TCGAACGGTCAGCCGGAGAATAATTATAAAACAACGCCACCC |
| | | | GTCCTGGATAGCGACGGCTCATTTTTTCTGTATAGCAAACTGA |
| | | | CTGTAGATAAATCACGGTGGCAGCAGGGCAATGTATTCAGTTG |
| | | | CTCCGTTATGCATGAAGCGTTACATAATCACTACACGCAGAAA |
| | | | TCTCTTAGTCTTTCACCCGGT |
| 1393 | ATX-P-3450 | HC IgG1 Fc nt | CAGGTGCAGCTGCAGGAGTCGGGCCCAGGGCTGGTGAAGCCT |
| | | | TCGGAGACCCTGTCCCTCACCTGCTCTGTCTCTGGTGACTCCGT |
| | | | CAGCAATAATCGTTATTATTGGAGCTGGATTCGGCAGCCCCCA |
| | | | GGGAAGGGACTGGAGTGGATTGGGTATATCTATTACAGTGGG |
| | | | AGCACCAACTACAACCCCTCCCTCAAGAGTCGAGTCACCATAT |
| | | | CAGTAGACACGTCCAAGAACCAGTTGTCCCTGAACCTGAGTTC |
| | | | TGTGACCGCTGCGGACACGGCCGTGTATTACTGTGCGAGAGAT |
| | | | CGCCCATCCCGGTATAAGTGGAATTACGATGCTTTTGCTATCT |
| | | | GGGGACAAGGGACAATGGTCACCGTCTCTTCAGCTAGCACTA |
| | | | AAGGGCCTTCTGTATTTCCCTTGGCCCCGTCCAGCAAATCGAC |
| | | | CTCGGGAGGGACAGCCGCCCTGGGTTGCCTTGTGAAAGATTAT |
| | | | TTCCCTGAGCCAGTTACCGTAAGTTGGAACAGTGGGGCGCTGA |
| | | | CAAGTGGTGTGCACACGTTTCCTGCCGTCCTGCAATCATCGGG |
| | | | CTTGTATAGCCTCAGCTCTGTGGTCACTGTCCCAAGTTCATCGC |
| | | | TGGGCACTCAGACGTATATTTGCAATGTGAACCACAAACCTTC |
| | | | AAATACAAAAGTGGATAAACGCGTAGAACCGAAATCGTGTGA |
| | | | TAAAACTCACACATGCCCGCCATGCCCGGCACCTGAACTGCTT |
| | | | GGTGGTCCCAGCGTGTTCCTGTTCCCGCCGAAGCCTAAAGATA |
| | | | CTCTAATGATCAGCCGTACGCCAGAGGTGACATGTGTCGTGGT |
| | | | TGACGTGTCCCACGAAGATCCCGAAGTTAAGTTCAATTGGTAT |
| | | | GTTGATGGTGTAGAGGTACACAATGCTAAGACTAAACCTCGCG |
| | | | AGGAGCAGTACGCCTCGACCTATCGTGTCGTGAGCGTTCTGAC |
| | | | CGTCCTTCACCAAGATTGGCTTAACGGCAAAGAATATAAGTGC |
| | | | AAGGTAAGCAATAAAGCACTTCCGGCCCCAATCGAGAAAACC |
| | | | ATTTCCAAGGCCAAAGGTCAACCAAGAGAACCCCAGGTGTAT |
| | | | ACTCTTCCGCCTTCTCGTGAGGAAATGACTAAAAATCAAGTAT |
| | | | CCCTTACGTGTCTGGTTAAAGGTTTTTATCCTAGCGATATTGCT |
| | | | GTTGAATGGGAATCGAACGGTCAGCCGGAGAATAATTATAAA |
| | | | ACAACGCCACCCGTCCTGGATAGCGACGGCTCATTTTTTCTGT |
| | | | ATAGCAAACTGACTGTAGATAAATCACGGTGGCAGCAGGGCA |
| | | | ATGTATTCAGTTGCTCCGTTATGCATGAAGCGTTACATAATCA |
| | | | CTACACGCAGAAATCTCTTAGTCTTTCACCCGGT |
| 1394 | ATX-P-3451 | HC IgG1 Fc nt | CAGGTGCAGCTACAGCAGTGGGGCGCAGGACTGTTGAAGCCT |
| | | | TCGGAGACCCTGTCCCTCACCTGCGCTGTCTATGGTGGGTCCTT |
| | | | CAGTGATTACTACTGGACCTGGATCCGCCAGCCCCCAGGGAAG |
| | | | GGGCTGGAGTGGATTGGTGAAGTCAATCATATTGGAAGCACC |
| | | | AACTACAACCCGTCCCTCAAGAGTCGAGTCACCATATCAGTAG |
| | | | ACACGTCCAAGAACCAGTTCTCCCTGAACCTGAGCTCTGTGAC |
| | | | CGCCGCGGACACGGCTGTGTATTACTGTGCGAAGTATAGCACT |
| | | | TCCTGGTACGGCTGGTTCGACCCCTGGGGCCAGGGAACCCTGG |
| | | | TCACTGTCTCCTCAGCTAGCACTAAAGGGCCTTCTGTATTTCCC |
| | | | TTGGCCCCGTCCAGCAAATCGACCTCGGGAGGGACAGCCGCC |
| | | | CTGGGTTGCCTTGTGAAAGATTATTTCCCTGAGCCAGTTACCG |
| | | | TAAGTTGGAACAGTGGGGCGCTGACAAGTGGTGTGCACACGT |
| | | | TTCCTGCCGTCCTGCAATCATCGGGCTTGTATAGCCTCAGCTCT |
| | | | GTGGTCACTGTCCCAAGTTCATCGCTGGGCACTCAGACGTATA |
| | | | TTTGCAATGTGAACCACAAACCTTCAAATACAAAAGTGGATAA |
| | | | ACGCGTAGAACCGAAATCGTGTGATAAAACTCACACATGCCC |
| | | | GCCATGCCCGGCACCTGAACTGCTTGGTGGTCCCAGCGTGTTC |
| | | | CTGTTCCCGCCGAAGCCTAAAGATACTCTAATGATCAGCCGTA |
| | | | CGCCAGAGGTGACATGTGTCGTGGTTGACGTGTCCCACGAAGA |
| | | | TCCCGAAGTTAAGTTCAATTGGTATGTTGATGGTGTAGAGGTA |
| | | | CACAATGCTAAGACTAAACCTCGCGAGGAGCAGTACGCCTCG |
| | | | ACCTATCGTGTCGTGAGCGTTCTGACCGTCCTTCACCAAGATT |
| | | | GGCTTAACGGCAAAGAATATAAGTGCAAGGTAAGCAATAAAG |
| | | | CACTTCCGGCCCCAATCGAGAAAACCATTTCCAAGGCCAAAG |
| | | | GTCAACCAAGAGAACCCCAGGTGTATACTCTTCCGCCTTCTCG |
| | | | TGAGGAAATGACTAAAAATCAAGTATCCCTTACGTGTCTGGTT |
| | | | AAAGGTTTTTATCCTAGCGATATTGCTGTTGAATGGGAATCGA |
| | | | ACGGTCAGCCGGAGAATAATTATAAAACAACGCCACCCGTCC |
| | | | TGGATAGCGACGGCTCATTTTTTCTGTATAGCAAACTGACTGT |
| | | | AGATAAATCACGGTGGCAGCAGGGCAATGTATTCAGTTGCTCC |
| | | | GTTATGCATGAAGCGTTACATAATCACTACACGCAGAAATCTC |
| | | | TTAGTCTTTCACCCGGT |
| 1395 | ATX-P-3453 | HC IgG1 Fc nt | CAGGTGCAGCTGCAGGAGTCGGGCCCAGGGCTGGTGAAGCCT |
| | | | TCGGGGACCCTGTCCCTCACCTGCGCTGTCTCTGGTGACTCCAT |
| | | | CAGCAGTAATAACTGGTGGAGTTGGTTCCGCCAGCCCCCAGGG |
| | | | AAGGGACTGGAGTGGATTGGGAGAATCTATCATAGTGGGACC |
| | | | ACCAACTACAACCCGTCCCTCGAGAGTCGAGTCACCATATCAG |
| | | | TAGACAACTCCAGGAACCAGTTCTCCCTGAAACTGAGCTCTGT |
| | | | GACCGCCGCGGACACGGCCGTGTATTACTGTGCGAGAGGCGA |
| | | | GGAGTGGGAGCTGCGGGGAGGTGTTTTTGATATCTGGGGACA |
| | | | AGGGACAATGGTCACCGTCTCTTCAGCTAGCACTAAAGGGCCT |
| | | | TCTGTATTTCCCTTGGCCCCGTCCAGCAAATCGACCTCGGGAG |
| | | | GGACAGCCGCCCTGGGTTGCCTTGTGAAAGATTATTTCCCTGA |
| | | | GCCAGTTACCGTAAGTTGGAACAGTGGGGCGCTGACAAGTGG |
| | | | TGTGCACACGTTTCCTGCCGTCCTGCAATCATCGGGCTTGTATA |
| | | | GCCTCAGCTCTGTGGTCACTGTCCCAAGTTCATCGCTGGGCAC |
| | | | TCAGACGTATATTTGCAATGTGAACCACAAACCTTCAAATACA |
| | | | AAAGTGGATAAACGCGTAGAACCGAAATCGTGTGATAAAACT |
| | | | CACACATGCCCGCCATGCCCGGCACCTGAACTGCTTGGTGGTC |
| | | | CCAGCGTGTTCCTGTTCCCGCCGAAGCCTAAAGATACTCTAAT |
| | | | GATCAGCCGTACGCCAGAGGTGACATGTGTCGTGGTTGACGTG |
| | | | TCCCACGAAGATCCCGAAGTTAAGTTCAATTGGTATGTTGATG |
| | | | GTGTAGAGGTACACAATGCTAAGACTAAACCTCGCGAGGAGC |
| | | | AGTACGCCTCGACCTATCGTGTCGTGAGCGTTCTGACCGTCCT |
| | | | TCACCAAGATTGGCTTAACGGCAAAGAATATAAGTGCAAGGT |
| | | | AAGCAATAAAGCACTTCCGGCCCCAATCGAGAAAACCATTTCC |
| | | | AAGGCCAAAGGTCAACCAAGAGAACCCCAGGTGTATACTCTT |
| | | | CCGCCTTCTCGTGAGGAAATGACTAAAAATCAAGTATCCCTTA |
| | | | CGTGTCTGGTTAAAGGTTTTTATCCTAGCGATATTGCTGTTGAA |
| | | | TGGGAATCGAACGGTCAGCCGGAGAATAATTATAAAACAACG |
| | | | CCACCCGTCCTGGATAGCGACGGCTCATTTTTTCTGTATAGCA |
| | | | AACTGACTGTAGATAAATCACGGTGGCAGCAGGGCAATGTAT |
| | | | TCAGTTGCTCCGTTATGCATGAAGCGTTACATAATCACTACAC |
| | | | GCAGAAATCTCTTAGTCTTTCACCCGGT |
| 1396 | ATX-P-3454 | HC IgG1 Fc nt | CAGGTGCAGCTGGTGCAATCTGGGCCTGAGTTGAAGAAGCCT |
| | | | GGGGCCTCAGTAAAGGTTTCCTGCAAGGCTTCTGGATACACCT |
| | | | TCACTAACTATGCTGTGAATTGGGTGCGACAGGCCCCTGGACA |
| | | | AGGGCTTGAGTGGATGGGATGGATCAACACCAACACTGGGCA |
| | | | CCCAACGTATGCCCAGGGCTTCACAGGACGGTTTGTCTTCTCC |
| | | | TTGGACACCTCTGTCAGTACGGCATATCTGCAGATCAGCAGCC |
| | | | TAAAGGCTGAGGACACTGCCGTTTATTACTGTGCGAGAGACAG |
| | | | CAGCAGCTGGGGCTACTGGGGCCAGGGAACCCTGGTCACTGT |
| | | | CTCCTCAGCTAGCACTAAAGGGCCTTCTGTATTTCCCTTGGCCC |
| | | | CGTCCAGCAAATCGACCTCGGGAGGGACAGCCGCCCTGGGTT |
| | | | GCCTTGTGAAAGATTATTTCCCTGAGCCAGTTACCGTAAGTTG |
| | | | GAACAGTGGGGCGCTGACAAGTGGTGTGCACACGTTTCCTGCC |
| | | | GTCCTGCAATCATCGGGCTTGTATAGCCTCAGCTCTGTGGTCA |
| | | | CTGTCCCAAGTTCATCGCTGGGCACTCAGACGTATATTTGCAA |
| | | | TGTGAACCACAAACCTTCAAATACAAAAGTGGATAAACGCGT |
| | | | AGAACCGAAATCGTGTGATAAAACTCACACATGCCCGCCATG |
| | | | CCCGGCACCTGAACTGCTTGGTGGTCCCAGCGTGTTCCTGTTC |
| | | | CCGCCGAAGCCTAAAGATACTCTAATGATCAGCCGTACGCCAG |
| | | | AGGTGACATGTGTCGTGGTTGACGTGTCCCACGAAGATCCCGA |
| | | | AGTTAAGTTCAATTGGTATGTTGATGGTGTAGAGGTACACAAT |
| | | | GCTAAGACTAAACCTCGCGAGGAGCAGTACGCCTCGACCTATC |
| | | | GTGTCGTGAGCGTTCTGACCGTCCTTCACCAAGATTGGCTTAA |
| | | | CGGCAAAGAATATAAGTGCAAGGTAAGCAATAAAGCACTTCC |
| | | | GGCCCCAATCGAGAAAACCATTTCCAAGGCCAAAGGTCAACC |
| | | | AAGAGAACCCCAGGTGTATACTCTTCCGCCTTCTCGTGAGGAA |
| | | | ATGACTAAAAATCAAGTATCCCTTACGTGTCTGGTTAAAGGTT |
| | | | TTTATCCTAGCGATATTGCTGTTGAATGGGAATCGAACGGTCA |
| | | | GCCGGAGAATAATTATAAAACAACGCCACCCGTCCTGGATAG |
| | | | CGACGGCTCATTTTTTCTGTATAGCAAACTGACTGTAGATAAA |
| | | | TCACGGTGGCAGCAGGGCAATGTATTCAGTTGCTCCGTTATGC |
| | | | ATGAAGCGTTACATAATCACTACACGCAGAAATCTCTTAGTCT |
| | | | TTCACCCGGT |
| 1397 | ATX-P-3455 | HC IgG1 Fc nt | CAGGTGCAGCTGCAGGAGTCGGGCCCAGGACTGGTGACGCCT |
| | | | TCGGGGACCCTGTCCCTCACCTGCGCTGTCTCTGGTGACTCCAT |
| | | | CAGTAGTGATAACTGGTGGAGTTGGGTCCGCCAGCCCCCAGG |
| | | | GAAGGGACTGGAGTGGATTGGGGAAATCTATCACAGTGAAAA |
| | | | CACCAACTACAACCCGTCCCTCAAGAGTCGAGTCACCATATCA |
| | | | GTAGACAAGTCCAAGAACCAGTTCTCCCTGAGGCTGAACTCTG |
| | | | TGACCGCCGCGGACACGGCCATGTATTACTGTGCGAGAGGGA |
| | | | GGGAGCTAACTGGCCTCTTTGACTACTGGGGCCAGGGAACCCT |
| | | | GGTCACCGTCTCCTCAGCTAGCACTAAAGGGCCTTCTGTATTT |
| | | | CCCTTGGCCCCGTCCAGCAAATCGACCTCGGGAGGGACAGCC |
| | | | GCCCTGGGTTGCCTTGTGAAAGATTATTTCCCTGAGCCAGTTA |
| | | | CCGTAAGTTGGAACAGTGGGGCGCTGACAAGTGGTGTGCACA |
| | | | CGTTTCCTGCCGTCCTGCAATCATCGGGCTTGTATAGCCTCAGC |
| | | | TCTGTGGTCACTGTCCCAAGTTCATCGCTGGGCACTCAGACGT |
| | | | ATATTTGCAATGTGAACCACAAACCTTCAAATACAAAAGTGGA |
| | | | TAAACGCGTAGAACCGAAATCGTGTGATAAAACTCACACATG |
| | | | CCCGCCATGCCCGGCACCTGAACTGCTTGGTGGTCCCAGCGTG |
| | | | TTCCTGTTCCCGCCGAAGCCTAAAGATACTCTAATGATCAGCC |
| | | | GTACGCCAGAGGTGACATGTGTCGTGGTTGACGTGTCCCACGA |
| | | | AGATCCCGAAGTTAAGTTCAATTGGTATGTTGATGGTGTAGAG |
| | | | GTACACAATGCTAAGACTAAACCTCGCGAGGAGCAGTACGCC |
| | | | TCGACCTATCGTGTCGTGAGCGTTCTGACCGTCCTTCACCAAG |
| | | | ATTGGCTTAACGGCAAAGAATATAAGTGCAAGGTAAGCAATA |
| | | | AAGCACTTCCGGCCCCAATCGAGAAAACCATTTCCAAGGCCA |
| | | | AAGGTCAACCAAGAGAACCCCAGGTGTATACTCTTCCGCCTTC |
| | | | TCGTGAGGAAATGACTAAAAATCAAGTATCCCTTACGTGTCTG |
| | | | GTTAAAGGTTTTTATCCTAGCGATATTGCTGTTGAATGGGAAT |
| | | | CGAACGGTCAGCCGGAGAATAATTATAAAACAACGCCACCCG |
| | | | TCCTGGATAGCGACGGCTCATTTTTTCTGTATAGCAAACTGAC |
| | | | TGTAGATAAATCACGGTGGCAGCAGGGCAATGTATTCAGTTGC |
| | | | TCCGTTATGCATGAAGCGTTACATAATCACTACACGCAGAAAT |
| | | | CTCTTAGTCTTTCACCCGGT |
| 1398 | ATX-P-3456 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGCAGTAATAACTGGTGGAGTTGGGTCCGCCAGCCCCCAGG |
| | | | AAAGGGACTGGAATGGATTGGGGACATCTATCATAGTGGAAA |
| | | | CACCAACTACAACCCGTCCCTCAAGAGTCGAGTCACCCTATCA |
| | | | GTAGACATGTCCAAGAACCAGTTCTCCCTGAAACTGAACTCTG |
| | | | TGACCGCCGCGGACACGGCCGTGTATTACTGTGCGAGAGGAG |
| | | | GTATAGCAGTGACTGCCTACTTTGACTACTGGGGCCAGGGAAC |
| | | | CCTGGTCACCGTCTCCTCAGCTAGCACTAAAGGGCCTTCTGTA |
| | | | TTTCCCTTGGCCCCGTCCAGCAAATCGACCTCGGGAGGGACAG |
| | | | CCGCCCTGGGTTGCCTTGTGAAAGATTATTTCCCTGAGCCAGT |
| | | | TACCGTAAGTTGGAACAGTGGGGCGCTGACAAGTGGTGTGCA |
| | | | CACGTTTCCTGCCGTCCTGCAATCATCGGGCTTGTATAGCCTCA |
| | | | GCTCTGTGGTCACTGTCCCAAGTTCATCGCTGGGCACTCAGAC |
| | | | GTATATTTGCAATGTGAACCACAAACCTTCAAATACAAAAGTG |
| | | | GATAAACGCGTAGAACCGAAATCGTGTGATAAAACTCACACA |
| | | | TGCCCGCCATGCCCGGCACCTGAACTGCTTGGTGGTCCCAGCG |
| | | | TGTTCCTGTTCCCGCCGAAGCCTAAAGATACTCTAATGATCAG |
| | | | CCGTACGCCAGAGGTGACATGTGTCGTGGTTGACGTGTCCCAC |
| | | | GAAGATCCCGAAGTTAAGTTCAATTGGTATGTTGATGGTGTAG |
| | | | AGGTACACAATGCTAAGACTAAACCTCGCGAGGAGCAGTACG |
| | | | CCTCGACCTATCGTGTCGTGAGCGTTCTGACCGTCCTTCACCA |
| | | | AGATTGGCTTAACGGCAAAGAATATAAGTGCAAGGTAAGCAA |
| | | | TAAAGCACTTCCGGCCCCAATCGAGAAAACCATTTCCAAGGCC |
| | | | AAAGGTCAACCAAGAGAACCCCAGGTGTATACTCTTCCGCCTT |
| | | | CTCGTGAGGAAATGACTAAAAATCAAGTATCCCTTACGTGTCT |
| | | | GGTTAAAGGTTTTTATCCTAGCGATATTGCTGTTGAATGGGAA |
| | | | TCGAACGGTCAGCCGGAGAATAATTATAAAACAACGCCACCC |
| | | | GTCCTGGATAGCGACGGCTCATTTTTTCTGTATAGCAAACTGA |
| | | | CTGTAGATAAATCACGGTGGCAGCAGGGCAATGTATTCAGTTG |
| | | | CTCCGTTATGCATGAAGCGTTACATAATCACTACACGCAGAAA |
| | | | TCTCTTAGTCTTTCACCCGGT |
| 1399 | ATX-P-3458 | HC IgG1 Fc nt | CAGGTGCAGCTGGTGCAGTCTGGGGCTGAGGTGAAGAAGCCT |
| | | | GGGGCCTCAGTGAAGGTCTCCTGCAAGGCTTCTGGATACACCT |
| | | | TCACCGGCTACTATTTGCACTGGGTGCGACAGGCCCCTGGACA |
| | | | AGGGCTTGAGTGGATGGGATGGATCAACCCTCACAGTGGTGG |
| | | | CACAATCTATGCACAGAAGTTTCAGGACAGGGTCACCATGACC |
| | | | AGGGACACGTCCATCAGCACAGCCTACATGGAGCTGAGCAGG |
| | | | CTGAGATCTGACGACACGGCCGTGTATTACTGTGCGAACTGGA |
| | | | ACTCCGGGGACTCCTACTACTACTACATGGACGTCTGGGGCAA |
| | | | AGGGACCACGGTCACCGTCTCCTCAGCTAGCACTAAAGGGCCT |
| | | | TCTGTATTTCCCTTGGCCCCGTCCAGCAAATCGACCTCGGGAG |
| | | | GGACAGCCGCCCTGGGTTGCCTTGTGAAAGATTATTTCCCTGA |
| | | | GCCAGTTACCGTAAGTTGGAACAGTGGGGCGCTGACAAGTGG |
| | | | TGTGCACACGTTTCCTGCCGTCCTGCAATCATCGGGCTTGTATA |
| | | | GCCTCAGCTCTGTGGTCACTGTCCCAAGTTCATCGCTGGGCAC |
| | | | TCAGACGTATATTTGCAATGTGAACCACAAACCTTCAAATACA |
| | | | AAAGTGGATAAACGCGTAGAACCGAAATCGTGTGATAAAACT |
| | | | CACACATGCCCGCCATGCCCGGCACCTGAACTGCTTGGTGGTC |
| | | | CCAGCGTGTTCCTGTTCCCGCCGAAGCCTAAAGATACTCTAAT |
| | | | GATCAGCCGTACGCCAGAGGTGACATGTGTCGTGGTTGACGTG |
| | | | TCCCACGAAGATCCCGAAGTTAAGTTCAATTGGTATGTTGATG |
| | | | GTGTAGAGGTACACAATGCTAAGACTAAACCTCGCGAGGAGC |
| | | | AGTACGCCTCGACCTATCGTGTCGTGAGCGTTCTGACCGTCCT |
| | | | TCACCAAGATTGGCTTAACGGCAAAGAATATAAGTGCAAGGT |
| | | | AAGCAATAAAGCACTTCCGGCCCCAATCGAGAAAACCATTTCC |
| | | | AAGGCCAAAGGTCAACCAAGAGAACCCCAGGTGTATACTCTT |
| | | | CCGCCTTCTCGTGAGGAAATGACTAAAAATCAAGTATCCCTTA |
| | | | CGTGTCTGGTTAAAGGTTTTTATCCTAGCGATATTGCTGTTGAA |
| | | | TGGGAATCGAACGGTCAGCCGGAGAATAATTATAAAACAACG |
| | | | CCACCCGTCCTGGATAGCGACGGCTCATTTTTTCTGTATAGCA |
| | | | AACTGACTGTAGATAAATCACGGTGGCAGCAGGGCAATGTAT |
| | | | TCAGTTGCTCCGTTATGCATGAAGCGTTACATAATCACTACAC |
| | | | GCAGAAATCTCTTAGTCTTTCACCCGGT |
| 1400 | ATX-P-3459 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGTTCCAT |
| | | | CAGCAGTGATAACTGGTGGAATTGGGTCCGCCAGCCCCCAGG |
| | | | GAAGGGGCTGGAGTGGATTGGAAAAATCTATCATAGTGGGAA |
| | | | CACCAACTACAACCCGTCCCTCAAGAGTCGAGTCACCATATCA |
| | | | GTAGACGAGTCCAAGAACCAGTTCTCCCTGAGGCTGTACTCTG |
| | | | TGACCGCCGCGGACACGGCCGTGTATTACTGTGCGAGAGGGG |
| | | | TCGATTTTTGGAGTGGTTCCCACATGGACGTCTGGGGCAAAGG |
| | | | GACCACGGTCACCGTCTCCTCAGCTAGCACTAAAGGGCCTTCT |
| | | | GTATTTCCCTTGGCCCCGTCCAGCAAATCGACCTCGGGAGGGA |
| | | | CAGCCGCCCTGGGTTGCCTTGTGAAAGATTATTTCCCTGAGCC |
| | | | AGTTACCGTAAGTTGGAACAGTGGGGCGCTGACAAGTGGTGT |
| | | | GCACACGTTTCCTGCCGTCCTGCAATCATCGGGCTTGTATAGC |
| | | | CTCAGCTCTGTGGTCACTGTCCCAAGTTCATCGCTGGGCACTC |
| | | | AGACGTATATTTGCAATGTGAACCACAAACCTTCAAATACAAA |
| | | | AGTGGATAAACGCGTAGAACCGAAATCGTGTGATAAAACTCA |
| | | | CACATGCCCGCCATGCCCGGCACCTGAACTGCTTGGTGGTCCC |
| | | | AGCGTGTTCCTGTTCCCGCCGAAGCCTAAAGATACTCTAATGA |
| | | | TCAGCCGTACGCCAGAGGTGACATGTGTCGTGGTTGACGTGTC |
| | | | CCACGAAGATCCCGAAGTTAAGTTCAATTGGTATGTTGATGGT |
| | | | GTAGAGGTACACAATGCTAAGACTAAACCTCGCGAGGAGCAG |
| | | | TACGCCTCGACCTATCGTGTCGTGAGCGTTCTGACCGTCCTTCA |
| | | | CCAAGATTGGCTTAACGGCAAAGAATATAAGTGCAAGGTAAG |
| | | | CAATAAAGCACTTCCGGCCCCAATCGAGAAAACCATTTCCAAG |
| | | | GCCAAAGGTCAACCAAGAGAACCCCAGGTGTATACTCTTCCGC |
| | | | CTTCTCGTGAGGAAATGACTAAAAATCAAGTATCCCTTACGTG |
| | | | TCTGGTTAAAGGTTTTTATCCTAGCGATATTGCTGTTGAATGGG |
| | | | AATCGAACGGTCAGCCGGAGAATAATTATAAAACAACGCCAC |
| | | | CCGTCCTGGATAGCGACGGCTCATTTTTTCTGTATAGCAAACT |
| | | | GACTGTAGATAAATCACGGTGGCAGCAGGGCAATGTATTCAG |
| | | | TTGCTCCGTTATGCATGAAGCGTTACATAATCACTACACGCAG |
| | | | AAATCTCTTAGTCTTTCACCCGGT |
| 1401 | ATX-P-3462 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | TAGCAGTAATAATTGGTGGAGTTGGGTCCGCCAGGCCCCAGG |
| | | | GAAGGGGCTGGAGTGGATTGGGAAGATCTATCAAAGTGGGAG |
| | | | CACCATGTACAACCCGTCCCTCAAGAGTCGAGTCACCATGTCG |
| | | | GTAGACAAGTCCAAGAACCAGTTCTCCCTGATTCTGAGTTCTG |
| | | | TGACCGCCGCGGACACGGCCGTGTATTACTGTGCGAGAGATA |
| | | | AACCCCCTTTACTGGGAGCCTTTGACTACTGGGGCCAGGGAAC |
| | | | CCTGGTCACTGTCTCCTCAGCTAGCACTAAAGGGCCTTCTGTA |
| | | | TTTCCCTTGGCCCCGTCCAGCAAATCGACCTCGGGAGGGACAG |
| | | | CCGCCCTGGGTTGCCTTGTGAAAGATTATTTCCCTGAGCCAGT |
| | | | TACCGTAAGTTGGAACAGTGGGGCGCTGACAAGTGGTGTGCA |
| | | | CACGTTTCCTGCCGTCCTGCAATCATCGGGCTTGTATAGCCTCA |
| | | | GCTCTGTGGTCACTGTCCCAAGTTCATCGCTGGGCACTCAGAC |
| | | | GTATATTTGCAATGTGAACCACAAACCTTCAAATACAAAAGTG |
| | | | GATAAACGCGTAGAACCGAAATCGTGTGATAAAACTCACACA |
| | | | TGCCCGCCATGCCCGGCACCTGAACTGCTTGGTGGTCCCAGCG |
| | | | TGTTCCTGTTCCCGCCGAAGCCTAAAGATACTCTAATGATCAG |
| | | | CCGTACGCCAGAGGTGACATGTGTCGTGGTTGACGTGTCCCAC |
| | | | GAAGATCCCGAAGTTAAGTTCAATTGGTATGTTGATGGTGTAG |
| | | | AGGTACACAATGCTAAGACTAAACCTCGCGAGGAGCAGTACG |
| | | | CCTCGACCTATCGTGTCGTGAGCGTTCTGACCGTCCTTCACCA |
| | | | AGATTGGCTTAACGGCAAAGAATATAAGTGCAAGGTAAGCAA |
| | | | TAAAGCACTTCCGGCCCCAATCGAGAAAACCATTTCCAAGGCC |
| | | | AAAGGTCAACCAAGAGAACCCCAGGTGTATACTCTTCCGCCTT |
| | | | CTCGTGAGGAAATGACTAAAAATCAAGTATCCCTTACGTGTCT |
| | | | GGTTAAAGGTTTTTATCCTAGCGATATTGCTGTTGAATGGGAA |
| | | | TCGAACGGTCAGCCGGAGAATAATTATAAAACAACGCCACCC |
| | | | GTCCTGGATAGCGACGGCTCATTTTTTCTGTATAGCAAACTGA |
| | | | CTGTAGATAAATCACGGTGGCAGCAGGGCAATGTATTCAGTTG |
| | | | CTCCGTTATGCATGAAGCGTTACATAATCACTACACGCAGAAA |
| | | | TCTCTTAGTCTTTCACCCGGT |
| 1402 | ATX-P-3463 | HC IgG1 Fc nt | CAGGTGCAGCTGGTGGAGTCTGGGGGAGGCGTGGTCCAGCCT |
| | | | GGGAGGTCCCTGAGACTCTCCTGTGCAGCCTCTGGATTCACCT |
| | | | TCAGTAGCTATGCCATGCACTGGGTCCGCCAGGCTCCAGGCAA |
| | | | GGGGCTGGAGTGGGTGGCAGTTATATCATATGATGGAAGTAA |
| | | | TAAATACTATGCGGACTCCGTGAAGGGCCGATTCACCATCTCC |
| | | | AGAGACAATTCCAAGAACACGCTGTCTCTGCAAATGAACAGC |
| | | | CTGAGAGCTGAGGACACGGCTGTGTATTACTGTGCGAAAAGG |
| | | | GGGGGATACAGCTATGGTCCTTTTGAACACTGGGGCCAGGGA |
| | | | ACCCTGGTCACCGTCTCCTCAGCTAGCACTAAAGGGCCTTCTG |
| | | | TATTTCCCTTGGCCCCGTCCAGCAAATCGACCTCGGGAGGGAC |
| | | | AGCCGCCCTGGGTTGCCTTGTGAAAGATTATTTCCCTGAGCCA |
| | | | GTTACCGTAAGTTGGAACAGTGGGGCGCTGACAAGTGGTGTG |
| | | | CACACGTTTCCTGCCGTCCTGCAATCATCGGGCTTGTATAGCCT |
| | | | CAGCTCTGTGGTCACTGTCCCAAGTTCATCGCTGGGCACTCAG |
| | | | ACGTATATTTGCAATGTGAACCACAAACCTTCAAATACAAAAG |
| | | | TGGATAAACGCGTAGAACCGAAATCGTGTGATAAAACTCACA |
| | | | CATGCCCGCCATGCCCGGCACCTGAACTGCTTGGTGGTCCCAG |
| | | | CGTGTTCCTGTTCCCGCCGAAGCCTAAAGATACTCTAATGATC |
| | | | AGCCGTACGCCAGAGGTGACATGTGTCGTGGTTGACGTGTCCC |
| | | | ACGAAGATCCCGAAGTTAAGTTCAATTGGTATGTTGATGGTGT |
| | | | AGAGGTACACAATGCTAAGACTAAACCTCGCGAGGAGCAGTA |
| | | | CGCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1403 | ATX-P-3466 | HC IgG1 Fc nt | CAGGTGCAGCTGGTGCAATCTGGGTCTGAATTGAAGAAGCCTG |
| | | | GGGCCTCAGTGAAGGTTTCCTGCAAGGCTTCTGGATACACCTT |
| | | | CACTAGCTATACTATGAATTGGGTGCGACAGGCCCCTGGACAA |
| | | | GGGCTTGAGTGGGTGGGATGGATCAACACCGCCACTGGGACC |
| | | | CCAACGTATGCCCAGGGCTTCACAGGACGGTATGTCTTCTCCT |
| | | | TGGACACCTCTGTCAGCACGGCTTATCTGCAGATCAGCGGCCT |
| | | | AAAGGCTGAGGACACTGCCGTGTATTTCTGTGCGAGGTATAGT |
| | | | GGGAGCCACTCCTTTGACTACTGGGGCCAGGGAACCCTGGTCA |
| | | | CCGTCTCTTCAGCTAGCACTAAAGGGCCTTCTGTATTTCCCTTG |
| | | | GCCCCGTCCAGCAAATCGACCTCGGGAGGGACAGCCGCCCTG |
| | | | GGTTGCCTTGTGAAAGATTATTTCCCTGAGCCAGTTACCGTAA |
| | | | GTTGGAACAGTGGGGCGCTGACAAGTGGTGTGCACACGTTTCC |
| | | | TGCCGTCCTGCAATCATCGGGCTTGTATAGCCTCAGCTCTGTG |
| | | | GTCACTGTCCCAAGTTCATCGCTGGGCACTCAGACGTATATTT |
| | | | GCAATGTGAACCACAAACCTTCAAATACAAAAGTGGATAAAC |
| | | | GCGTAGAACCGAAATCGTGTGATAAAACTCACACATGCCCGC |
| | | | CATGCCCGGCACCTGAACTGCTTGGTGGTCCCAGCGTGTTCCT |
| | | | GTTCCCGCCGAAGCCTAAAGATACTCTAATGATCAGCCGTACG |
| | | | CCAGAGGTGACATGTGTCGTGGTTGACGTGTCCCACGAAGATC |
| | | | CCGAAGTTAAGTTCAATTGGTATGTTGATGGTGTAGAGGTACA |
| | | | CAATGCTAAGACTAAACCTCGCGAGGAGCAGTACGCCTCGAC |
| | | | CTATCGTGTCGTGAGCGTTCTGACCGTCCTTCACCAAGATTGG |
| | | | CTTAACGGCAAAGAATATAAGTGCAAGGTAAGCAATAAAGCA |
| | | | CTTCCGGCCCCAATCGAGAAAACCATTTCCAAGGCCAAAGGTC |
| | | | AACCAAGAGAACCCCAGGTGTATACTCTTCCGCCTTCTCGTGA |
| | | | GGAAATGACTAAAAATCAAGTATCCCTTACGTGTCTGGTTAAA |
| | | | GGTTTTTATCCTAGCGATATTGCTGTTGAATGGGAATCGAACG |
| | | | GTCAGCCGGAGAATAATTATAAAACAACGCCACCCGTCCTGG |
| | | | ATAGCGACGGCTCATTTTTTCTGTATAGCAAACTGACTGTAGA |
| | | | TAAATCACGGTGGCAGCAGGGCAATGTATTCAGTTGCTCCGTT |
| | | | ATGCATGAAGCGTTACATAATCACTACACGCAGAAATCTCTTA |
| | | | GTCTTTCACCCGGT |
| 1404 | ATX-P-3467 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGTAGTGATTACTGGTGGAGTTGGGTCCGCCAGTCCCCAGGG |
| | | | AAGGGGCTGGAGTGGATTGGGAAAATCTATCATAGTGGGAAC |
| | | | ACCAACCACAACCCGTCCCTCAAGAGTCGAGTCACCATATCAA |
| | | | TAGACAAGTCCAGGAACCAGTTCTCCCTGAATCTGAACTCTGT |
| | | | GACCGCCGCGGACACGGCCGTGTATTACTGTGCGAGAGAAGG |
| | | | ACTGGAACTTCCCCACATGGACGTCTGGGGCAAAGGGACCAC |
| | | | GGTCACCGTCTCCTCAGCTAGCACTAAAGGGCCTTCTGTATTT |
| | | | CCCTTGGCCCCGTCCAGCAAATCGACCTCGGGAGGGACAGCC |
| | | | GCCCTGGGTTGCCTTGTGAAAGATTATTTCCCTGAGCCAGTTA |
| | | | CCGTAAGTTGGAACAGTGGGGCGCTGACAAGTGGTGTGCACA |
| | | | CGTTTCCTGCCGTCCTGCAATCATCGGGCTTGTATAGCCTCAGC |
| | | | TCTGTGGTCACTGTCCCAAGTTCATCGCTGGGCACTCAGACGT |
| | | | ATATTTGCAATGTGAACCACAAACCTTCAAATACAAAAGTGGA |
| | | | TAAACGCGTAGAACCGAAATCGTGTGATAAAACTCACACATG |
| | | | CCCGCCATGCCCGGCACCTGAACTGCTTGGTGGTCCCAGCGTG |
| | | | TTCCTGTTCCCGCCGAAGCCTAAAGATACTCTAATGATCAGCC |
| | | | GTACGCCAGAGGTGACATGTGTCGTGGTTGACGTGTCCCACGA |
| | | | AGATCCCGAAGTTAAGTTCAATTGGTATGTTGATGGTGTAGAG |
| | | | GTACACAATGCTAAGACTAAACCTCGCGAGGAGCAGTACGCC |
| | | | TCGACCTATCGTGTCGTGAGCGTTCTGACCGTCCTTCACCAAG |
| | | | ATTGGCTTAACGGCAAAGAATATAAGTGCAAGGTAAGCAATA |
| | | | AAGCACTTCCGGCCCCAATCGAGAAAACCATTTCCAAGGCCA |
| | | | AAGGTCAACCAAGAGAACCCCAGGTGTATACTCTTCCGCCTTC |
| | | | TCGTGAGGAAATGACTAAAAATCAAGTATCCCTTACGTGTCTG |
| | | | GTTAAAGGTTTTTATCCTAGCGATATTGCTGTTGAATGGGAAT |
| | | | CGAACGGTCAGCCGGAGAATAATTATAAAACAACGCCACCCG |
| | | | TCCTGGATAGCGACGGCTCATTTTTTCTGTATAGCAAACTGAC |
| | | | TGTAGATAAATCACGGTGGCAGCAGGGCAATGTATTCAGTTGC |
| | | | TCCGTTATGCATGAAGCGTTACATAATCACTACACGCAGAAAT |
| | | | CTCTTAGTCTTTCACCCGGT |
| 1405 | ATX-P-3468 | HC IgG1 Fc nt | CAGGTGCAGCTGGTGGAGTCTGGGGGAGGCTTGGTCCAGCCT |
| | | | GGGAGGTCCCTGAGACTCTCCTGTGCAGCCTCTGGATTCACCT |
| | | | TCAGTAGTTATGCTATGCACTGGGTCCGCCAGGCTCCAGGCAA |
| | | | GGGGCTGGAGTGGGTGACAGTTATATCATATGATGGAAGTAA |
| | | | TAAATACTATGCAGACTCCGTGAAGGGCCGATTCACCATCTCC |
| | | | AGAGACAATTCCAAGAACACGCTGTATCTGCAAATGAACAGC |
| | | | CTGAGAGCTGAGGACACGGCTGTGTATTACTGTGCGAAAAGG |
| | | | GGTAACTGGAACTACGGACCTTTTCACTACTGGGGCCAGGGAA |
| | | | CCCTGGTCACTGTCTCCTCAGCTAGCACTAAAGGGCCTTCTGT |
| | | | ATTTCCCTTGGCCCCGTCCAGCAAATCGACCTCGGGAGGGACA |
| | | | GCCGCCCTGGGTTGCCTTGTGAAAGATTATTTCCCTGAGCCAG |
| | | | TTACCGTAAGTTGGAACAGTGGGGCGCTGACAAGTGGTGTGC |
| | | | ACACGTTTCCTGCCGTCCTGCAATCATCGGGCTTGTATAGCCTC |
| | | | AGCTCTGTGGTCACTGTCCCAAGTTCATCGCTGGGCACTCAGA |
| | | | CGTATATTTGCAATGTGAACCACAAACCTTCAAATACAAAAGT |
| | | | GGATAAACGCGTAGAACCGAAATCGTGTGATAAAACTCACAC |
| | | | ATGCCCGCCATGCCCGGCACCTGAACTGCTTGGTGGTCCCAGC |
| | | | GTGTTCCTGTTCCCGCCGAAGCCTAAAGATACTCTAATGATCA |
| | | | GCCGTACGCCAGAGGTGACATGTGTCGTGGTTGACGTGTCCCA |
| | | | CGAAGATCCCGAAGTTAAGTTCAATTGGTATGTTGATGGTGTA |
| | | | GAGGTACACAATGCTAAGACTAAACCTCGCGAGGAGCAGTAC |
| | | | GCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1406 | ATX-P-3469 | HC IgG1 Fc nt | CAGGTGCAGCTGGTGGAGTCTGGGGGAGGCGTGGTCCAGCCT |
| | | | GGGAGGTCCCTGAGACTCTCCTGTGCAGCCTCTGGATTCACCT |
| | | | TCAGTGCCTATGCCATCCACTGGGTCCGCCAGGCTCCAGGCAA |
| | | | GGGGCTGGAGTGGGTGGCAGTTATATCATATGATGGAAGTAA |
| | | | TAAAAAGTATGCAGACTCCGTGAAGGGCCGATTCACCATCTCC |
| | | | AGAGACAATTCCAAGAACACGCTGGATCTGGAAATGAACAGC |
| | | | CTGAGAGCTGAGGACACGGCTGTGTACTACTGTGCGAAAGGG |
| | | | CTACCTTTTGACTACTGGGGCCAGGGAACCCTGGTCACCGTCT |
| | | | CCTCAGCTAGCACTAAAGGGCCTTCTGTATTTCCCTTGGCCCC |
| | | | GTCCAGCAAATCGACCTCGGGAGGGACAGCCGCCCTGGGTTG |
| | | | CCTTGTGAAAGATTATTTCCCTGAGCCAGTTACCGTAAGTTGG |
| | | | AACAGTGGGGCGCTGACAAGTGGTGTGCACACGTTTCCTGCCG |
| | | | TCCTGCAATCATCGGGCTTGTATAGCCTCAGCTCTGTGGTCACT |
| | | | GTCCCAAGTTCATCGCTGGGCACTCAGACGTATATTTGCAATG |
| | | | TGAACCACAAACCTTCAAATACAAAAGTGGATAAACGCGTAG |
| | | | AACCGAAATCGTGTGATAAAACTCACACATGCCCGCCATGCCC |
| | | | GGCACCTGAACTGCTTGGTGGTCCCAGCGTGTTCCTGTTCCCG |
| | | | CCGAAGCCTAAAGATACTCTAATGATCAGCCGTACGCCAGAG |
| | | | GTGACATGTGTCGTGGTTGACGTGTCCCACGAAGATCCCGAAG |
| | | | TTAAGTTCAATTGGTATGTTGATGGTGTAGAGGTACACAATGC |
| | | | TAAGACTAAACCTCGCGAGGAGCAGTACGCCTCGACCTATCGT |
| | | | GTCGTGAGCGTTCTGACCGTCCTTCACCAAGATTGGCTTAACG |
| | | | GCAAAGAATATAAGTGCAAGGTAAGCAATAAAGCACTTCCGG |
| | | | CCCCAATCGAGAAAACCATTTCCAAGGCCAAAGGTCAACCAA |
| | | | GAGAACCCCAGGTGTATACTCTTCCGCCTTCTCGTGAGGAAAT |
| | | | GACTAAAAATCAAGTATCCCTTACGTGTCTGGTTAAAGGTTTT |
| | | | TATCCTAGCGATATTGCTGTTGAATGGGAATCGAACGGTCAGC |
| | | | CGGAGAATAATTATAAAACAACGCCACCCGTCCTGGATAGCG |
| | | | ACGGCTCATTTTTTCTGTATAGCAAACTGACTGTAGATAAATC |
| | | | ACGGTGGCAGCAGGGCAATGTATTCAGTTGCTCCGTTATGCAT |
| | | | GAAGCGTTACATAATCACTACACGCAGAAATCTCTTAGTCTTT |
| | | | CACCCGGT |
| 1407 | ATX-P-3470 | HC IgG1 Fc nt | CAGGTGCAGCTGGTGCAATCTGGGTCTGACTTGAAGAAGCCTG |
| | | | GGGCCTCAGTGAAGGTTTCCTGCAAGGCTTCTGGATACACCTT |
| | | | CACTAGTTATACTATGAATTGGGTGCGACAGGCCCCTGGACAA |
| | | | GGGCTTGAGTGGATGGGATGGATCAACACCAACACTGGGACC |
| | | | CCAACGTATGCCCAGGGCTTCACAGGACGGTTTGTCTTCTCCT |
| | | | TGGACACCTCTGTCAGCACGGCATATCTGCAGATCAGCAGCCT |
| | | | AAAGGCTGAGGACACTGCCGTGTATTACTGTACGAGGTATACT |
| | | | GGGAACTACTCCTTTGACTACTGGGGCCAGGGAACCCTGGTCA |
| | | | CCGTCTCCTCAGCTAGCACTAAAGGGCCTTCTGTATTTCCCTTG |
| | | | GCCCCGTCCAGCAAATCGACCTCGGGAGGGACAGCCGCCCTG |
| | | | GGTTGCCTTGTGAAAGATTATTTCCCTGAGCCAGTTACCGTAA |
| | | | GTTGGAACAGTGGGGCGCTGACAAGTGGTGTGCACACGTTTCC |
| | | | TGCCGTCCTGCAATCATCGGGCTTGTATAGCCTCAGCTCTGTG |
| | | | GTCACTGTCCCAAGTTCATCGCTGGGCACTCAGACGTATATTT |
| | | | GCAATGTGAACCACAAACCTTCAAATACAAAAGTGGATAAAC |
| | | | GCGTAGAACCGAAATCGTGTGATAAAACTCACACATGCCCGC |
| | | | CATGCCCGGCACCTGAACTGCTTGGTGGTCCCAGCGTGTTCCT |
| | | | GTTCCCGCCGAAGCCTAAAGATACTCTAATGATCAGCCGTACG |
| | | | CCAGAGGTGACATGTGTCGTGGTTGACGTGTCCCACGAAGATC |
| | | | CCGAAGTTAAGTTCAATTGGTATGTTGATGGTGTAGAGGTACA |
| | | | CAATGCTAAGACTAAACCTCGCGAGGAGCAGTACGCCTCGAC |
| | | | CTATCGTGTCGTGAGCGTTCTGACCGTCCTTCACCAAGATTGG |
| | | | CTTAACGGCAAAGAATATAAGTGCAAGGTAAGCAATAAAGCA |
| | | | CTTCCGGCCCCAATCGAGAAAACCATTTCCAAGGCCAAAGGTC |
| | | | AACCAAGAGAACCCCAGGTGTATACTCTTCCGCCTTCTCGTGA |
| | | | GGAAATGACTAAAAATCAAGTATCCCTTACGTGTCTGGTTAAA |
| | | | GGTTTTTATCCTAGCGATATTGCTGTTGAATGGGAATCGAACG |
| | | | GTCAGCCGGAGAATAATTATAAAACAACGCCACCCGTCCTGG |
| | | | ATAGCGACGGCTCATTTTTTCTGTATAGCAAACTGACTGTAGA |
| | | | TAAATCACGGTGGCAGCAGGGCAATGTATTCAGTTGCTCCGTT |
| | | | ATGCATGAAGCGTTACATAATCACTACACGCAGAAATCTCTTA |
| | | | GTCTTTCACCCGGT |
| 1408 | ATX-P-3471 | HC IgG1 Fc nt | GAGGTGCAGCTGGTGGAGTCTGGGGGAGTCTTGGTACAGCCT |
| | | | GGGGGGTCCCTGAGACTCTCCTGTGCAGCCTCTGGATTCACCT |
| | | | TTAGGAGCTATGCCATGAGCTGGGTCCGCCAGGCTCCAGGGA |
| | | | AGGGGCTGGAGTGGGTCTCAGCTATTAGTGGTAGTGGTGATAG |
| | | | CACATACTTCGCAGACTCCGTGAAGGGCCGGTTCACCATCTCC |
| | | | AGAGACAATTCCAAGAACACGCTGTCTCTGCAAATGAACAGC |
| | | | CTGAGAGCCGAGGACACGGCCGTATATTACTGTGCGAGCATCC |
| | | | TAGGATATTGTAGTGGTACCAGCTGCTATGGCCTCTTTGACAA |
| | | | CTGGGGCCAGGGAACCCTGGTCACCGTCTCCTCAGCTAGCACT |
| | | | AAAGGGCCTTCTGTATTTCCCTTGGCCCCGTCCAGCAAATCGA |
| | | | CCTCGGGAGGGACAGCCGCCCTGGGTTGCCTTGTGAAAGATTA |
| | | | TTTCCCTGAGCCAGTTACCGTAAGTTGGAACAGTGGGGCGCTG |
| | | | ACAAGTGGTGTGCACACGTTTCCTGCCGTCCTGCAATCATCGG |
| | | | GCTTGTATAGCCTCAGCTCTGTGGTCACTGTCCCAAGTTCATCG |
| | | | CTGGGCACTCAGACGTATATTTGCAATGTGAACCACAAACCTT |
| | | | CAAATACAAAAGTGGATAAACGCGTAGAACCGAAATCGTGTG |
| | | | ATAAAACTCACACATGCCCGCCATGCCCGGCACCTGAACTGCT |
| | | | TGGTGGTCCCAGCGTGTTCCTGTTCCCGCCGAAGCCTAAAGAT |
| | | | ACTCTAATGATCAGCCGTACGCCAGAGGTGACATGTGTCGTGG |
| | | | TTGACGTGTCCCACGAAGATCCCGAAGTTAAGTTCAATTGGTA |
| | | | TGTTGATGGTGTAGAGGTACACAATGCTAAGACTAAACCTCGC |
| | | | GAGGAGCAGTACGCCTCGACCTATCGTGTCGTGAGCGTTCTGA |
| | | | CCGTCCTTCACCAAGATTGGCTTAACGGCAAAGAATATAAGTG |
| | | | CAAGGTAAGCAATAAAGCACTTCCGGCCCCAATCGAGAAAAC |
| | | | CATTTCCAAGGCCAAAGGTCAACCAAGAGAACCCCAGGTGTA |
| | | | TACTCTTCCGCCTTCTCGTGAGGAAATGACTAAAAATCAAGTA |
| | | | TCCCTTACGTGTCTGGTTAAAGGTTTTTATCCTAGCGATATTGC |
| | | | TGTTGAATGGGAATCGAACGGTCAGCCGGAGAATAATTATAA |
| | | | AACAACGCCACCCGTCCTGGATAGCGACGGCTCATTTTTTCTG |
| | | | TATAGCAAACTGACTGTAGATAAATCACGGTGGCAGCAGGGC |
| | | | AATGTATTCAGTTGCTCCGTTATGCATGAAGCGTTACATAATC |
| | | | ACTACACGCAGAAATCTCTTAGTCTTTCACCCGGT |
| 1409 | ATX-P-3472 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGGCTCCAT |
| | | | CAGTAATGATAACTGGTGGAGTTGGGTCCGCCAGTCCCCCGGG |
| | | | AAGGGGCTGGAGTGGATTGGGAAAATCTATCATAGTGAGTTC |
| | | | ACCAACTACAACCCGTCCCTCAGGAGTCGAGTCACCATATCAG |
| | | | TAGACAAGTCCAAGAACCAGTTCTCCCTGGAGCTGACCTCTGT |
| | | | GACCGCCGCGGACACGGCCGTGTATTACTGTGCGAGAGAGTA |
| | | | TCAGAACTACATGGACGTCTGGGGCAAAGGGACCACGGTCAC |
| | | | CGTCTCCTCAGCTAGCACTAAAGGGCCTTCTGTATTTCCCTTGG |
| | | | CCCCGTCCAGCAAATCGACCTCGGGAGGGACAGCCGCCCTGG |
| | | | GTTGCCTTGTGAAAGATTATTTCCCTGAGCCAGTTACCGTAAG |
| | | | TTGGAACAGTGGGGCGCTGACAAGTGGTGTGCACACGTTTCCT |
| | | | GCCGTCCTGCAATCATCGGGCTTGTATAGCCTCAGCTCTGTGG |
| | | | TCACTGTCCCAAGTTCATCGCTGGGCACTCAGACGTATATTTG |
| | | | CAATGTGAACCACAAACCTTCAAATACAAAAGTGGATAAACG |
| | | | CGTAGAACCGAAATCGTGTGATAAAACTCACACATGCCCGCC |
| | | | ATGCCCGGCACCTGAACTGCTTGGTGGTCCCAGCGTGTTCCTG |
| | | | TTCCCGCCGAAGCCTAAAGATACTCTAATGATCAGCCGTACGC |
| | | | CAGAGGTGACATGTGTCGTGGTTGACGTGTCCCACGAAGATCC |
| | | | CGAAGTTAAGTTCAATTGGTATGTTGATGGTGTAGAGGTACAC |
| | | | AATGCTAAGACTAAACCTCGCGAGGAGCAGTACGCCTCGACC |
| | | | TATCGTGTCGTGAGCGTTCTGACCGTCCTTCACCAAGATTGGC |
| | | | TTAACGGCAAAGAATATAAGTGCAAGGTAAGCAATAAAGCAC |
| | | | TTCCGGCCCCAATCGAGAAAACCATTTCCAAGGCCAAAGGTCA |
| | | | ACCAAGAGAACCCCAGGTGTATACTCTTCCGCCTTCTCGTGAG |
| | | | GAAATGACTAAAAATCAAGTATCCCTTACGTGTCTGGTTAAAG |
| | | | GTTTTTATCCTAGCGATATTGCTGTTGAATGGGAATCGAACGG |
| | | | TCAGCCGGAGAATAATTATAAAACAACGCCACCCGTCCTGGAT |
| | | | AGCGACGGCTCATTTTTTCTGTATAGCAAACTGACTGTAGATA |
| | | | AATCACGGTGGCAGCAGGGCAATGTATTCAGTTGCTCCGTTAT |
| | | | GCATGAAGCGTTACATAATCACTACACGCAGAAATCTCTTAGT |
| | | | CTTTCACCCGGT |
| 1410 | ATX-P-3361 | HC IgG1 Fc nt | CAGGTGCAGCTGCAGGAGTCGGGCCCAGGACTGGTGAAGCCT |
| | | | TCGGGGACCCTGTCCCTCACCTGCGTTGTCTCTGGTGACTCCAT |
| | | | CAGCAGTAATAACTGGTGGAGTTGGGTCCGCCAGCCCCCAGG |
| | | | GAAGGGTCTGGAGTGGATTGGACAAATCTATCATAGTGGAAT |
| | | | CACCAACTACAACCCGTCCCTCAAGAGTCGAGTCACCATATCA |
| | | | GTAGACAAGTCCAAGAACCAGTTCTCCCTGGAGCTGACCTCTG |
| | | | TGACCGCCGCGGACGCGGCCGTGTATTACTGTGCGAGGGCCCA |
| | | | GTGGGAACTACGAGCGGGCTGGTTCGACCCCTGGGGCCAGGG |
| | | | AACCCTGGTCACCGTCTCCTCAGCTAGCACTAAAGGGCCTTCT |
| | | | GTATTTCCCTTGGCCCCGTCCAGCAAATCGACCTCGGGAGGGA |
| | | | CAGCCGCCCTGGGTTGCCTTGTGAAAGATTATTTCCCTGAGCC |
| | | | AGTTACCGTAAGTTGGAACAGTGGGGCGCTGACAAGTGGTGT |
| | | | GCACACGTTTCCTGCCGTCCTGCAATCATCGGGCTTGTATAGC |
| | | | CTCAGCTCTGTGGTCACTGTCCCAAGTTCATCGCTGGGCACTC |
| | | | AGACGTATATTTGCAATGTGAACCACAAACCTTCAAATACAAA |
| | | | AGTGGATAAACGCGTAGAACCGAAATCGTGTGATAAAACTCA |
| | | | CACATGCCCGCCATGCCCGGCACCTGAACTGCTTGGTGGTCCC |
| | | | AGCGTGTTCCTGTTCCCGCCGAAGCCTAAAGATACTCTAATGA |
| | | | TCAGCCGTACGCCAGAGGTGACATGTGTCGTGGTTGACGTGTC |
| | | | CCACGAAGATCCCGAAGTTAAGTTCAATTGGTATGTTGATGGT |
| | | | GTAGAGGTACACAATGCTAAGACTAAACCTCGCGAGGAGCAG |
| | | | TACGCCTCGACCTATCGTGTCGTGAGCGTTCTGACCGTCCTTCA |
| | | | CCAAGATTGGCTTAACGGCAAAGAATATAAGTGCAAGGTAAG |
| | | | CAATAAAGCACTTCCGGCCCCAATCGAGAAAACCATTTCCAAG |
| | | | GCCAAAGGTCAACCAAGAGAACCCCAGGTGTATACTCTTCCGC |
| | | | CTTCTCGTGAGGAAATGACTAAAAATCAAGTATCCCTTACGTG |
| | | | TCTGGTTAAAGGTTTTTATCCTAGCGATATTGCTGTTGAATGGG |
| | | | AATCGAACGGTCAGCCGGAGAATAATTATAAAACAACGCCAC |
| | | | CCGTCCTGGATAGCGACGGCTCATTTTTTCTGTATAGCAAACT |
| | | | GACTGTAGATAAATCACGGTGGCAGCAGGGCAATGTATTCAG |
| | | | TTGCTCCGTTATGCATGAAGCGTTACATAATCACTACACGCAG |
| | | | AAATCTCTTAGTCTTTCACCCGGT |
| 1411 | ATX-P-3366 | HC IgG1 Fc nt | GAGGTGCAGCTGGTGGAGTCTGGGGGAGGCTTGGTAAAGCCT |
| | | | GGGGGGACCCTTACACTCTCCTGTGCAGCCTCTGGATTCACTT |
| | | | TCAGTAACGCCTGGATGAGCTGGGTCCGCCAGGCTCCAGGGA |
| | | | AGGGGCTGGAGTGGGTTGGCCGTATTAAAAGCAAAACTGATG |
| | | | GCGGGACAACAGACTACGCTGCACCCGTGAAAGGCAGATTCA |
| | | | CCATCTCAAGAGATGATTCAAAAAACACGCTGTATCTGCAAAT |
| | | | GAACAGCCTGAAAACCGAGGACACAGCCGTGTATTACTGTAC |
| | | | CCCCCGGTACAACTCGGACTTTGACTACTGGGGCCAAGGAACC |
| | | | CTGGTCACCGTCTCTTCAGCTAGCACTAAAGGGCCTTCTGTATT |
| | | | TCCCTTGGCCCCGTCCAGCAAATCGACCTCGGGAGGGACAGCC |
| | | | GCCCTGGGTTGCCTTGTGAAAGATTATTTCCCTGAGCCAGTTA |
| | | | CCGTAAGTTGGAACAGTGGGGCGCTGACAAGTGGTGTGCACA |
| | | | CGTTTCCTGCCGTCCTGCAATCATCGGGCTTGTATAGCCTCAGC |
| | | | TCTGTGGTCACTGTCCCAAGTTCATCGCTGGGCACTCAGACGT |
| | | | ATATTTGCAATGTGAACCACAAACCTTCAAATACAAAAGTGGA |
| | | | TAAACGCGTAGAACCGAAATCGTGTGATAAAACTCACACATG |
| | | | CCCGCCATGCCCGGCACCTGAACTGCTTGGTGGTCCCAGCGTG |
| | | | TTCCTGTTCCCGCCGAAGCCTAAAGATACTCTAATGATCAGCC |
| | | | GTACGCCAGAGGTGACATGTGTCGTGGTTGACGTGTCCCACGA |
| | | | AGATCCCGAAGTTAAGTTCAATTGGTATGTTGATGGTGTAGAG |
| | | | GTACACAATGCTAAGACTAAACCTCGCGAGGAGCAGTACGCC |
| | | | TCGACCTATCGTGTCGTGAGCGTTCTGACCGTCCTTCACCAAG |
| | | | ATTGGCTTAACGGCAAAGAATATAAGTGCAAGGTAAGCAATA |
| | | | AAGCACTTCCGGCCCCAATCGAGAAAACCATTTCCAAGGCCA |
| | | | AAGGTCAACCAAGAGAACCCCAGGTGTATACTCTTCCGCCTTC |
| | | | TCGTGAGGAAATGACTAAAAATCAAGTATCCCTTACGTGTCTG |
| | | | GTTAAAGGTTTTTATCCTAGCGATATTGCTGTTGAATGGGAAT |
| | | | CGAACGGTCAGCCGGAGAATAATTATAAAACAACGCCACCCG |
| | | | TCCTGGATAGCGACGGCTCATTTTTTCTGTATAGCAAACTGAC |
| | | | TGTAGATAAATCACGGTGGCAGCAGGGCAATGTATTCAGTTGC |
| | | | TCCGTTATGCATGAAGCGTTACATAATCACTACACGCAGAAAT |
| | | | CTCTTAGTCTTTCACCCGGT |
| 1412 | ATX-P-3376 | HC IgG1 Fc nt | CAGGTGCAGCTGCAGGAGTCGGGCCCAGGACTGGTGAAGCCT |
| | | | TCGGAGACCCTGTCCCTCACCTGCACTGTCTCTGGTGGCTCCAT |
| | | | CAGTAGTTACTACTGGAGCTGGATCCGGCAGCCCCCAGGGAA |
| | | | GGGACTGGAGTGGATTGGGTATATCTATTACAGTGGGTTCACC |
| | | | AAGTACAACCCCTCCCTCAAGAGTCGAGTCACCATATCAGTAG |
| | | | ACACGTCCAAGAACCAGTTCTCCCTGAAGCTGAGCTCTGTGAC |
| | | | CGCTGCGGACACGGCCGTGTATTACTGTGCGACCCACCCTCGT |
| | | | GGATACAACTATGGTCCCTTTGACTACTGGGGCCAGGGAACCC |
| | | | TGGTCACTGTCTCCTCAGCTAGCACTAAAGGGCCTTCTGTATTT |
| | | | CCCTTGGCCCCGTCCAGCAAATCGACCTCGGGAGGGACAGCC |
| | | | GCCCTGGGTTGCCTTGTGAAAGATTATTTCCCTGAGCCAGTTA |
| | | | CCGTAAGTTGGAACAGTGGGGCGCTGACAAGTGGTGTGCACA |
| | | | CGTTTCCTGCCGTCCTGCAATCATCGGGCTTGTATAGCCTCAGC |
| | | | TCTGTGGTCACTGTCCCAAGTTCATCGCTGGGCACTCAGACGT |
| | | | ATATTTGCAATGTGAACCACAAACCTTCAAATACAAAAGTGGA |
| | | | TAAACGCGTAGAACCGAAATCGTGTGATAAAACTCACACATG |
| | | | CCCGCCATGCCCGGCACCTGAACTGCTTGGTGGTCCCAGCGTG |
| | | | TTCCTGTTCCCGCCGAAGCCTAAAGATACTCTAATGATCAGCC |
| | | | GTACGCCAGAGGTGACATGTGTCGTGGTTGACGTGTCCCACGA |
| | | | AGATCCCGAAGTTAAGTTCAATTGGTATGTTGATGGTGTAGAG |
| | | | GTACACAATGCTAAGACTAAACCTCGCGAGGAGCAGTACGCC |
| | | | TCGACCTATCGTGTCGTGAGCGTTCTGACCGTCCTTCACCAAG |
| | | | ATTGGCTTAACGGCAAAGAATATAAGTGCAAGGTAAGCAATA |
| | | | AAGCACTTCCGGCCCCAATCGAGAAAACCATTTCCAAGGCCA |
| | | | AAGGTCAACCAAGAGAACCCCAGGTGTATACTCTTCCGCCTTC |
| | | | TCGTGAGGAAATGACTAAAAATCAAGTATCCCTTACGTGTCTG |
| | | | GTTAAAGGTTTTTATCCTAGCGATATTGCTGTTGAATGGGAAT |
| | | | CGAACGGTCAGCCGGAGAATAATTATAAAACAACGCCACCCG |
| | | | TCCTGGATAGCGACGGCTCATTTTTTCTGTATAGCAAACTGAC |
| | | | TGTAGATAAATCACGGTGGCAGCAGGGCAATGTATTCAGTTGC |
| | | | TCCGTTATGCATGAAGCGTTACATAATCACTACACGCAGAAAT |
| | | | CTCTTAGTCTTTCACCCGGT |
| 1413 | ATX-P-3377 | HC IgG1 Fc nt | CAGGTGCAGCTGCAGGAGTCGGGCCCAGGACTGGTGAAGCCT |
| | | | TCGGAGACCCTGTCCCTCACCTGCACTGTCTCTGGTGGCTCCGT |
| | | | CAACAGTGGTCGTTACTACTGGAGCTGGATCCGGCAGCCCCCA |
| | | | GGGAAGGGACTGGAGTGGATTGGGTATATCTATTCCAGTGGGT |
| | | | ACACCAACTACAACCCCTCCCTCAAGAGTCGAGTCACCATGTT |
| | | | ACTAGACACGTCCAAGAACCAGTTCTCCCTGAAGCTGAACTCC |
| | | | GTGACCGCTGCGGACACGGCCGTGTATTACTGTGCGAGGTCAC |
| | | | GTGGATACCGCTATGGTCCCTTTGACTACTGGGGCCAGGGAAC |
| | | | CCTGGTCACCGTCTCCTCAGCTAGCACTAAAGGGCCTTCTGTA |
| | | | TTTCCCTTGGCCCCGTCCAGCAAATCGACCTCGGGAGGGACAG |
| | | | CCGCCCTGGGTTGCCTTGTGAAAGATTATTTCCCTGAGCCAGT |
| | | | TACCGTAAGTTGGAACAGTGGGGCGCTGACAAGTGGTGTGCA |
| | | | CACGTTTCCTGCCGTCCTGCAATCATCGGGCTTGTATAGCCTCA |
| | | | GCTCTGTGGTCACTGTCCCAAGTTCATCGCTGGGCACTCAGAC |
| | | | GTATATTTGCAATGTGAACCACAAACCTTCAAATACAAAAGTG |
| | | | GATAAACGCGTAGAACCGAAATCGTGTGATAAAACTCACACA |
| | | | TGCCCGCCATGCCCGGCACCTGAACTGCTTGGTGGTCCCAGCG |
| | | | TGTTCCTGTTCCCGCCGAAGCCTAAAGATACTCTAATGATCAG |
| | | | CCGTACGCCAGAGGTGACATGTGTCGTGGTTGACGTGTCCCAC |
| | | | GAAGATCCCGAAGTTAAGTTCAATTGGTATGTTGATGGTGTAG |
| | | | AGGTACACAATGCTAAGACTAAACCTCGCGAGGAGCAGTACG |
| | | | CCTCGACCTATCGTGTCGTGAGCGTTCTGACCGTCCTTCACCA |
| | | | AGATTGGCTTAACGGCAAAGAATATAAGTGCAAGGTAAGCAA |
| | | | TAAAGCACTTCCGGCCCCAATCGAGAAAACCATTTCCAAGGCC |
| | | | AAAGGTCAACCAAGAGAACCCCAGGTGTATACTCTTCCGCCTT |
| | | | CTCGTGAGGAAATGACTAAAAATCAAGTATCCCTTACGTGTCT |
| | | | GGTTAAAGGTTTTTATCCTAGCGATATTGCTGTTGAATGGGAA |
| | | | TCGAACGGTCAGCCGGAGAATAATTATAAAACAACGCCACCC |
| | | | GTCCTGGATAGCGACGGCTCATTTTTTCTGTATAGCAAACTGA |
| | | | CTGTAGATAAATCACGGTGGCAGCAGGGCAATGTATTCAGTTG |
| | | | CTCCGTTATGCATGAAGCGTTACATAATCACTACACGCAGAAA |
| | | | TCTCTTAGTCTTTCACCCGGT |
| 1414 | ATX-P-3418 | HC IgG1 Fc nt | CAGGTGCAGCTGGTGCAATCTGGGTCTGAGTTGAAGAAGCCTG |
| | | | GGGCCTCAGTGAAGGTTTCCTGCGAGGCTTCTGGATACACCTT |
| | | | CACTAGCTATGCTATGAATTGGGTGCGACAGGCCCCTGGACAA |
| | | | GGGCTTGAGTGGATGGGATGGATCAACACCAACACTGGGACC |
| | | | CCAACTTATGCCCAGGGCTTCACAGGACGGTTTGTCTTCTCCTT |
| | | | GGACACCTCTGTCAGCACGGCATATCTGCAGATCAGCAGCCTA |
| | | | AAGGCTGAGGACACTGCCGTGTATTACTGTGCGAGTCATAGCA |
| | | | CCTCGTTTGACTACTGGGGCCAGGGGACCCTGGTCACCGTCTC |
| | | | CTCAGCTAGCACTAAAGGGCCTTCTGTATTTCCCTTGGCCCCGT |
| | | | CCAGCAAATCGACCTCGGGAGGGACAGCCGCCCTGGGTTGCC |
| | | | TTGTGAAAGATTATTTCCCTGAGCCAGTTACCGTAAGTTGGAA |
| | | | CAGTGGGGCGCTGACAAGTGGTGTGCACACGTTTCCTGCCGTC |
| | | | CTGCAATCATCGGGCTTGTATAGCCTCAGCTCTGTGGTCACTG |
| | | | TCCCAAGTTCATCGCTGGGCACTCAGACGTATATTTGCAATGT |
| | | | GAACCACAAACCTTCAAATACAAAAGTGGATAAACGCGTAGA |
| | | | ACCGAAATCGTGTGATAAAACTCACACATGCCCGCCATGCCCG |
| | | | GCACCTGAACTGCTTGGTGGTCCCAGCGTGTTCCTGTTCCCGC |
| | | | CGAAGCCTAAAGATACTCTAATGATCAGCCGTACGCCAGAGG |
| | | | TGACATGTGTCGTGGTTGACGTGTCCCACGAAGATCCCGAAGT |
| | | | TAAGTTCAATTGGTATGTTGATGGTGTAGAGGTACACAATGCT |
| | | | AAGACTAAACCTCGCGAGGAGCAGTACGCCTCGACCTATCGT |
| | | | GTCGTGAGCGTTCTGACCGTCCTTCACCAAGATTGGCTTAACG |
| | | | GCAAAGAATATAAGTGCAAGGTAAGCAATAAAGCACTTCCGG |
| | | | CCCCAATCGAGAAAACCATTTCCAAGGCCAAAGGTCAACCAA |
| | | | GAGAACCCCAGGTGTATACTCTTCCGCCTTCTCGTGAGGAAAT |
| | | | GACTAAAAATCAAGTATCCCTTACGTGTCTGGTTAAAGGTTTT |
| | | | TATCCTAGCGATATTGCTGTTGAATGGGAATCGAACGGTCAGC |
| | | | CGGAGAATAATTATAAAACAACGCCACCCGTCCTGGATAGCG |
| | | | ACGGCTCATTTTTTCTGTATAGCAAACTGACTGTAGATAAATC |
| | | | ACGGTGGCAGCAGGGCAATGTATTCAGTTGCTCCGTTATGCAT |
| | | | GAAGCGTTACATAATCACTACACGCAGAAATCTCTTAGTCTTT |
| | | | CACCCGGT |
| 1415 | ATX-P-3420 | HC IgG1 Fc nt | CAGGTGCAGCTACAGCAGTGGGGCGCAGGACTGGTGAAGCCT |
| | | | TCGGAGACCCTGTCCCTCACCTGCGCTGTCCATGGTGGGTCCT |
| | | | TCAGTGATTACTACTTGACCTGGATTCGCCAGCCCCCAGGGAA |
| | | | GGGGCTGGAGTGGATTGGGGAAATCAATCATAGTGGAAGCAC |
| | | | CAACTACAACCCCTCCCTCAAGAGTCGAGTCACCTTATCAATA |
| | | | GACACGTCCAAGAACCAGTTCTCCCTGAAGCTGAGCTCTGTGA |
| | | | CCGCCGCGGACACGGCTGTGTATTACTGTGCGAGAGGTAGTGA |
| | | | AGACTACTACTATTACATGGACGTCTGGGGCAAAGGGACCAC |
| | | | GGTCACCGTCTCCTCAGCTAGCACTAAAGGGCCTTCTGTATTT |
| | | | CCCTTGGCCCCGTCCAGCAAATCGACCTCGGGAGGGACAGCC |
| | | | GCCCTGGGTTGCCTTGTGAAAGATTATTTCCCTGAGCCAGTTA |
| | | | CCGTAAGTTGGAACAGTGGGGCGCTGACAAGTGGTGTGCACA |
| | | | CGTTTCCTGCCGTCCTGCAATCATCGGGCTTGTATAGCCTCAGC |
| | | | TCTGTGGTCACTGTCCCAAGTTCATCGCTGGGCACTCAGACGT |
| | | | ATATTTGCAATGTGAACCACAAACCTTCAAATACAAAAGTGGA |
| | | | TAAACGCGTAGAACCGAAATCGTGTGATAAAACTCACACATG |
| | | | CCCGCCATGCCCGGCACCTGAACTGCTTGGTGGTCCCAGCGTG |
| | | | TTCCTGTTCCCGCCGAAGCCTAAAGATACTCTAATGATCAGCC |
| | | | GTACGCCAGAGGTGACATGTGTCGTGGTTGACGTGTCCCACGA |
| | | | AGATCCCGAAGTTAAGTTCAATTGGTATGTTGATGGTGTAGAG |
| | | | GTACACAATGCTAAGACTAAACCTCGCGAGGAGCAGTACGCC |
| | | | TCGACCTATCGTGTCGTGAGCGTTCTGACCGTCCTTCACCAAG |
| | | | ATTGGCTTAACGGCAAAGAATATAAGTGCAAGGTAAGCAATA |
| | | | AAGCACTTCCGGCCCCAATCGAGAAAACCATTTCCAAGGCCA |
| | | | AAGGTCAACCAAGAGAACCCCAGGTGTATACTCTTCCGCCTTC |
| | | | TCGTGAGGAAATGACTAAAAATCAAGTATCCCTTACGTGTCTG |
| | | | GTTAAAGGTTTTTATCCTAGCGATATTGCTGTTGAATGGGAAT |
| | | | CGAACGGTCAGCCGGAGAATAATTATAAAACAACGCCACCCG |
| | | | TCCTGGATAGCGACGGCTCATTTTTTCTGTATAGCAAACTGAC |
| | | | TGTAGATAAATCACGGTGGCAGCAGGGCAATGTATTCAGTTGC |
| | | | TCCGTTATGCATGAAGCGTTACATAATCACTACACGCAGAAAT |
| | | | CTCTTAGTCTTTCACCCGGT |
| 1416 | ATX-P-3426 | HC IgG1 Fc nt | GAGGTGCAGCTGGTGCAGTCTGGAGCAGAGGTGAAAAAGCCC |
| | | | GGGGAGTCTCTGAAGATCTCCTGTAAGGGTTCTGGATACATCT |
| | | | TTACCAGCTACTGGATCGGCTGGGTGCGCCAGATGCCCGGGAA |
| | | | AGGCCTGGAGTGGATGGGGATCATCTATCCTGGTGACTCTGAT |
| | | | ACCAGATACAGCCCGTCTTTCCAAGGCCAGGTCACCATCTCAG |
| | | | CCGACAAGTCCATCATCACCGCCTACCTGCAGTGGAGCAGCCT |
| | | | GAAGGCCTCGGACACCGCCATGTATTACTGTGCGAGACACGA |
| | | | CTACTTTGATATTGGAGGTTATTTTGACTACTGGGGCCAGGGA |
| | | | ACCCTGGTCACCGTCTCCTCAGCTAGCACTAAAGGGCCTTCTG |
| | | | TATTTCCCTTGGCCCCGTCCAGCAAATCGACCTCGGGAGGGAC |
| | | | AGCCGCCCTGGGTTGCCTTGTGAAAGATTATTTCCCTGAGCCA |
| | | | GTTACCGTAAGTTGGAACAGTGGGGCGCTGACAAGTGGTGTG |
| | | | CACACGTTTCCTGCCGTCCTGCAATCATCGGGCTTGTATAGCCT |
| | | | CAGCTCTGTGGTCACTGTCCCAAGTTCATCGCTGGGCACTCAG |
| | | | ACGTATATTTGCAATGTGAACCACAAACCTTCAAATACAAAAG |
| | | | TGGATAAACGCGTAGAACCGAAATCGTGTGATAAAACTCACA |
| | | | CATGCCCGCCATGCCCGGCACCTGAACTGCTTGGTGGTCCCAG |
| | | | CGTGTTCCTGTTCCCGCCGAAGCCTAAAGATACTCTAATGATC |
| | | | AGCCGTACGCCAGAGGTGACATGTGTCGTGGTTGACGTGTCCC |
| | | | ACGAAGATCCCGAAGTTAAGTTCAATTGGTATGTTGATGGTGT |
| | | | AGAGGTACACAATGCTAAGACTAAACCTCGCGAGGAGCAGTA |
| | | | CGCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1417 | ATX-P-3432 | HC IgG1 Fc nt | CAGGTGCAGCTGGTGCAGTCTGGGGCTGAGGTGAAGAAGCCT |
| | | | GGGGCCTCAGTGAAGGTCTCCTGCAAGGCTGATGGATACACCT |
| | | | TCACCGACTACTATATGCACTGGGTGCGACAGGCCCCTGGACA |
| | | | AGGGCATGAGTGGATGGGATGGATCAACCCTAACAGTGGTGT |
| | | | CACATACTATGCACAGAAGTTTCAGGGCAGGGTCACCATGACC |
| | | | AGGGACACGTCCATCAGCACAGCCTACATGGAGCTGAACAGA |
| | | | CTTAGATCTGACGACACGGCCGTGTATTACTGTGCGAGAGATC |
| | | | GGACCTTCTACTTTGACTACTGGGGCCAGGGAACCCTGGTCAC |
| | | | TGTCTCCTCAGCTAGCACTAAAGGGCCTTCTGTATTTCCCTTGG |
| | | | CCCCGTCCAGCAAATCGACCTCGGGAGGGACAGCCGCCCTGG |
| | | | GTTGCCTTGTGAAAGATTATTTCCCTGAGCCAGTTACCGTAAG |
| | | | TTGGAACAGTGGGGCGCTGACAAGTGGTGTGCACACGTTTCCT |
| | | | GCCGTCCTGCAATCATCGGGCTTGTATAGCCTCAGCTCTGTGG |
| | | | TCACTGTCCCAAGTTCATCGCTGGGCACTCAGACGTATATTTG |
| | | | CAATGTGAACCACAAACCTTCAAATACAAAAGTGGATAAACG |
| | | | CGTAGAACCGAAATCGTGTGATAAAACTCACACATGCCCGCC |
| | | | ATGCCCGGCACCTGAACTGCTTGGTGGTCCCAGCGTGTTCCTG |
| | | | TTCCCGCCGAAGCCTAAAGATACTCTAATGATCAGCCGTACGC |
| | | | CAGAGGTGACATGTGTCGTGGTTGACGTGTCCCACGAAGATCC |
| | | | CGAAGTTAAGTTCAATTGGTATGTTGATGGTGTAGAGGTACAC |
| | | | AATGCTAAGACTAAACCTCGCGAGGAGCAGTACGCCTCGACC |
| | | | TATCGTGTCGTGAGCGTTCTGACCGTCCTTCACCAAGATTGGC |
| | | | TTAACGGCAAAGAATATAAGTGCAAGGTAAGCAATAAAGCAC |
| | | | TTCCGGCCCCAATCGAGAAAACCATTTCCAAGGCCAAAGGTCA |
| | | | ACCAAGAGAACCCCAGGTGTATACTCTTCCGCCTTCTCGTGAG |
| | | | GAAATGACTAAAAATCAAGTATCCCTTACGTGTCTGGTTAAAG |
| | | | GTTTTTATCCTAGCGATATTGCTGTTGAATGGGAATCGAACGG |
| | | | TCAGCCGGAGAATAATTATAAAACAACGCCACCCGTCCTGGAT |
| | | | AGCGACGGCTCATTTTTTCTGTATAGCAAACTGACTGTAGATA |
| | | | AATCACGGTGGCAGCAGGGCAATGTATTCAGTTGCTCCGTTAT |
| | | | GCATGAAGCGTTACATAATCACTACACGCAGAAATCTCTTAGT |
| | | | CTTTCACCCGGT |
| 1418 | ATX-P-3433 | HC IgG1 Fc nt | CAGGTGCAGCTGGTGCAGTCTGGGGCTGAGGTGAAGAAGCCT |
| | | | GGGGCCTCAGTGAAGGTCTCCTGCAAGGCTTCTGGATACACCT |
| | | | TCACCGACTACTATATGCACTGGGTGCGACAGGCCCCTGGACA |
| | | | AGGGCTTGAGTGGATGGGATGGATCAACCCTAACAATGGTGG |
| | | | CACAGACTATGCACAGAAGTTTCAGGGCAGGGTCACCATGAC |
| | | | CAGGGACACGTCCATCAGCACAGCCTACATGGAGTTACACAG |
| | | | GCTAAGATCTGACGACACGGCCGTGTATTTCTGTGCGAGAGGG |
| | | | GTCTATGATGTAGTAGTTCCAACTGCTATCGTCAACTGGTTCG |
| | | | ACCCCTGGGGCCAGGGAACCCTGGTCACCGTCTCCTCAGCTAG |
| | | | CACTAAAGGGCCTTCTGTATTTCCCTTGGCCCCGTCCAGCAAA |
| | | | TCGACCTCGGGAGGGACAGCCGCCCTGGGTTGCCTTGTGAAAG |
| | | | ATTATTTCCCTGAGCCAGTTACCGTAAGTTGGAACAGTGGGGC |
| | | | GCTGACAAGTGGTGTGCACACGTTTCCTGCCGTCCTGCAATCA |
| | | | TCGGGCTTGTATAGCCTCAGCTCTGTGGTCACTGTCCCAAGTTC |
| | | | ATCGCTGGGCACTCAGACGTATATTTGCAATGTGAACCACAAA |
| | | | CCTTCAAATACAAAAGTGGATAAACGCGTAGAACCGAAATCG |
| | | | TGTGATAAAACTCACACATGCCCGCCATGCCCGGCACCTGAAC |
| | | | TGCTTGGTGGTCCCAGCGTGTTCCTGTTCCCGCCGAAGCCTAA |
| | | | AGATACTCTAATGATCAGCCGTACGCCAGAGGTGACATGTGTC |
| | | | GTGGTTGACGTGTCCCACGAAGATCCCGAAGTTAAGTTCAATT |
| | | | GGTATGTTGATGGTGTAGAGGTACACAATGCTAAGACTAAACC |
| | | | TCGCGAGGAGCAGTACGCCTCGACCTATCGTGTCGTGAGCGTT |
| | | | CTGACCGTCCTTCACCAAGATTGGCTTAACGGCAAAGAATATA |
| | | | AGTGCAAGGTAAGCAATAAAGCACTTCCGGCCCCAATCGAGA |
| | | | AAACCATTTCCAAGGCCAAAGGTCAACCAAGAGAACCCCAGG |
| | | | TGTATACTCTTCCGCCTTCTCGTGAGGAAATGACTAAAAATCA |
| | | | AGTATCCCTTACGTGTCTGGTTAAAGGTTTTTATCCTAGCGATA |
| | | | TTGCTGTTGAATGGGAATCGAACGGTCAGCCGGAGAATAATTA |
| | | | TAAAACAACGCCACCCGTCCTGGATAGCGACGGCTCATTTTTT |
| | | | CTGTATAGCAAACTGACTGTAGATAAATCACGGTGGCAGCAG |
| | | | GGCAATGTATTCAGTTGCTCCGTTATGCATGAAGCGTTACATA |
| | | | ATCACTACACGCAGAAATCTCTTAGTCTTTCACCCGGT |
| 1419 | ATX-P-3438 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGACTCCAT |
| | | | CAGCAATGATTACTGGTGGAATTGGGTCCGCCAGCCCCCAGGG |
| | | | AAGGGTCTGGAGTGGATTGGAGAAATCTATCATAGTGGGCAC |
| | | | ACCAACTACAACCCGTCCCTCAAGAGTCGAGTCACCATATCAG |
| | | | TAGACAAGTCCAAGAACCAGTTGTCCCTGAAGCTGACCTCTGT |
| | | | GAACGCCGCGGACACGGCCGTGTATTACTGTGCGAGAGGTGG |
| | | | AACTGAAATTACGGGGTACATGGACGTCTGGGGCAAAGGGAC |
| | | | CACGGTCACCGTCTCCTCAGCTAGCACTAAAGGGCCTTCTGTA |
| | | | TTTCCCTTGGCCCCGTCCAGCAAATCGACCTCGGGAGGGACAG |
| | | | CCGCCCTGGGTTGCCTTGTGAAAGATTATTTCCCTGAGCCAGT |
| | | | TACCGTAAGTTGGAACAGTGGGGCGCTGACAAGTGGTGTGCA |
| | | | CACGTTTCCTGCCGTCCTGCAATCATCGGGCTTGTATAGCCTCA |
| | | | GCTCTGTGGTCACTGTCCCAAGTTCATCGCTGGGCACTCAGAC |
| | | | GTATATTTGCAATGTGAACCACAAACCTTCAAATACAAAAGTG |
| | | | GATAAACGCGTAGAACCGAAATCGTGTGATAAAACTCACACA |
| | | | TGCCCGCCATGCCCGGCACCTGAACTGCTTGGTGGTCCCAGCG |
| | | | TGTTCCTGTTCCCGCCGAAGCCTAAAGATACTCTAATGATCAG |
| | | | CCGTACGCCAGAGGTGACATGTGTCGTGGTTGACGTGTCCCAC |
| | | | GAAGATCCCGAAGTTAAGTTCAATTGGTATGTTGATGGTGTAG |
| | | | AGGTACACAATGCTAAGACTAAACCTCGCGAGGAGCAGTACG |
| | | | CCTCGACCTATCGTGTCGTGAGCGTTCTGACCGTCCTTCACCA |
| | | | AGATTGGCTTAACGGCAAAGAATATAAGTGCAAGGTAAGCAA |
| | | | TAAAGCACTTCCGGCCCCAATCGAGAAAACCATTTCCAAGGCC |
| | | | AAAGGTCAACCAAGAGAACCCCAGGTGTATACTCTTCCGCCTT |
| | | | CTCGTGAGGAAATGACTAAAAATCAAGTATCCCTTACGTGTCT |
| | | | GGTTAAAGGTTTTTATCCTAGCGATATTGCTGTTGAATGGGAA |
| | | | TCGAACGGTCAGCCGGAGAATAATTATAAAACAACGCCACCC |
| | | | GTCCTGGATAGCGACGGCTCATTTTTTCTGTATAGCAAACTGA |
| | | | CTGTAGATAAATCACGGTGGCAGCAGGGCAATGTATTCAGTTG |
| | | | CTCCGTTATGCATGAAGCGTTACATAATCACTACACGCAGAAA |
| | | | TCTCTTAGTCTTTCACCCGGT |
| 1420 | ATX-P-3441 | HC IgG1 Fc nt | CAGGTGCAGCTGCAGGAGTCGGGCCCAGGACTGGTGAAGCCT |
| | | | TCGGGGACCCTGTCCCTCACCTGCGCTGTCTCTGGTGCCTCCAT |
| | | | CAGAAGTGGTCACTGGTGGAGTTGGGTCCGCCAGCCCCCAGG |
| | | | GAAGGGACTGGAGTGGATTGGGGAAATCTATCATAGTGGGAA |
| | | | CACCAACTACAACCCGTCCCTCAAGAGTCGAGTCACCCTATCA |
| | | | GTAGACAAGTCCAAGAACCAGTTCTCCCTGAAGCTGAGCTCTG |
| | | | TGACCGCCGCGGACACGGCCGTGTATTACTGTGCGAGAGGTCA |
| | | | CTACGGTGACTACGTCGTCTACTTTGACTACTGGGGCCAGGGA |
| | | | ACCCTGGTCACTGTCTCCTCAGCTAGCACTAAAGGGCCTTCTG |
| | | | TATTTCCCTTGGCCCCGTCCAGCAAATCGACCTCGGGAGGGAC |
| | | | AGCCGCCCTGGGTTGCCTTGTGAAAGATTATTTCCCTGAGCCA |
| | | | GTTACCGTAAGTTGGAACAGTGGGGCGCTGACAAGTGGTGTG |
| | | | CACACGTTTCCTGCCGTCCTGCAATCATCGGGCTTGTATAGCCT |
| | | | CAGCTCTGTGGTCACTGTCCCAAGTTCATCGCTGGGCACTCAG |
| | | | ACGTATATTTGCAATGTGAACCACAAACCTTCAAATACAAAAG |
| | | | TGGATAAACGCGTAGAACCGAAATCGTGTGATAAAACTCACA |
| | | | CATGCCCGCCATGCCCGGCACCTGAACTGCTTGGTGGTCCCAG |
| | | | CGTGTTCCTGTTCCCGCCGAAGCCTAAAGATACTCTAATGATC |
| | | | AGCCGTACGCCAGAGGTGACATGTGTCGTGGTTGACGTGTCCC |
| | | | ACGAAGATCCCGAAGTTAAGTTCAATTGGTATGTTGATGGTGT |
| | | | AGAGGTACACAATGCTAAGACTAAACCTCGCGAGGAGCAGTA |
| | | | CGCCTCGACCTATCGTGTCGTGAGCGTTCTGACCGTCCTTCACC |
| | | | AAGATTGGCTTAACGGCAAAGAATATAAGTGCAAGGTAAGCA |
| | | | ATAAAGCACTTCCGGCCCCAATCGAGAAAACCATTTCCAAGGC |
| | | | CAAAGGTCAACCAAGAGAACCCCAGGTGTATACTCTTCCGCCT |
| | | | TCTCGTGAGGAAATGACTAAAAATCAAGTATCCCTTACGTGTC |
| | | | TGGTTAAAGGTTTTTATCCTAGCGATATTGCTGTTGAATGGGA |
| | | | ATCGAACGGTCAGCCGGAGAATAATTATAAAACAACGCCACC |
| | | | CGTCCTGGATAGCGACGGCTCATTTTTTCTGTATAGCAAACTG |
| | | | ACTGTAGATAAATCACGGTGGCAGCAGGGCAATGTATTCAGTT |
| | | | GCTCCGTTATGCATGAAGCGTTACATAATCACTACACGCAGAA |
| | | | ATCTCTTAGTCTTTCACCCGGT |
| 1421 | ATX-P-3446 | HC IgG1 Fc nt | CAGGTGCAGCTGCAGGAGTCGGGCCCAGGACTGGTGAAGCCT |
| | | | TCGGAGACCCTGTCCCTCACCTGCACTGTCTCTGGTGGCTCCAT |
| | | | CAGTAGTTACTATTGGAGCTGGATCCGGCAGCCCCCAGGGAA |
| | | | GGGACTGGAATGGATTGGTTATGTCTATTACAGTGGGTACACC |
| | | | AAGTACAATCCCTCCCTCAAGAGTCGAGTCACCATATCAATAG |
| | | | ACGCGTCCAAGAACCAGTTCTCCCTGAAGCTGACCTCTGTGAC |
| | | | CGCTGCGGACACGGCCGTGTATTACTGTGCGACCGTCGAATCA |
| | | | AAGTGGAACTACGGGCCTTTTGCTATCTGGGGACAAGGGACA |
| | | | ATGGTCACCGTCTCTTCAGCTAGCACTAAAGGGCCTTCTGTAT |
| | | | TTCCCTTGGCCCCGTCCAGCAAATCGACCTCGGGAGGGACAGC |
| | | | CGCCCTGGGTTGCCTTGTGAAAGATTATTTCCCTGAGCCAGTT |
| | | | ACCGTAAGTTGGAACAGTGGGGCGCTGACAAGTGGTGTGCAC |
| | | | ACGTTTCCTGCCGTCCTGCAATCATCGGGCTTGTATAGCCTCA |
| | | | GCTCTGTGGTCACTGTCCCAAGTTCATCGCTGGGCACTCAGAC |
| | | | GTATATTTGCAATGTGAACCACAAACCTTCAAATACAAAAGTG |
| | | | GATAAACGCGTAGAACCGAAATCGTGTGATAAAACTCACACA |
| | | | TGCCCGCCATGCCCGGCACCTGAACTGCTTGGTGGTCCCAGCG |
| | | | TGTTCCTGTTCCCGCCGAAGCCTAAAGATACTCTAATGATCAG |
| | | | CCGTACGCCAGAGGTGACATGTGTCGTGGTTGACGTGTCCCAC |
| | | | GAAGATCCCGAAGTTAAGTTCAATTGGTATGTTGATGGTGTAG |
| | | | AGGTACACAATGCTAAGACTAAACCTCGCGAGGAGCAGTACG |
| | | | CCTCGACCTATCGTGTCGTGAGCGTTCTGACCGTCCTTCACCA |
| | | | AGATTGGCTTAACGGCAAAGAATATAAGTGCAAGGTAAGCAA |
| | | | TAAAGCACTTCCGGCCCCAATCGAGAAAACCATTTCCAAGGCC |
| | | | AAAGGTCAACCAAGAGAACCCCAGGTGTATACTCTTCCGCCTT |
| | | | CTCGTGAGGAAATGACTAAAAATCAAGTATCCCTTACGTGTCT |
| | | | GGTTAAAGGTTTTTATCCTAGCGATATTGCTGTTGAATGGGAA |
| | | | TCGAACGGTCAGCCGGAGAATAATTATAAAACAACGCCACCC |
| | | | GTCCTGGATAGCGACGGCTCATTTTTTCTGTATAGCAAACTGA |
| | | | CTGTAGATAAATCACGGTGGCAGCAGGGCAATGTATTCAGTTG |
| | | | CTCCGTTATGCATGAAGCGTTACATAATCACTACACGCAGAAA |
| | | | TCTCTTAGTCTTTCACCCGGT |
| 1422 | ATX-P-3452 | HC IgG1 Fc nt | CAGGTGCAGCTGGTGCAGTCTGGGGCTGAGGTGAAGAAGCCT |
| | | | GGGGCCTCAGTGAAGGTCTCCTGCAAGGCTTCTGGATACACCT |
| | | | TCACCGGCTACTATATGAACTGGGTGCGACAGGCCCCTGGACA |
| | | | AGGGCTTGAGTGGATGGGATGGATCAACCCTAACAGTGGTGC |
| | | | CACAAACTATGCACAGAAGTTTCAGGGCAGGGTCACCATGAC |
| | | | CAGGGACACGTCCATCAGCACAGACTACATGGAACTGAGCAG |
| | | | GCTGAGATCTGACGACACGGCCGTTTATTACTGTGCGAGAGAT |
| | | | GGGGCTAAGTGGAATCACGAGGGTTACTACTTTGACTACTGGG |
| | | | GCCAGGGAACCCTGGTCACCGTCTCCTCAGCTAGCACTAAAGG |
| | | | GCCTTCTGTATTTCCCTTGGCCCCGTCCAGCAAATCGACCTCGG |
| | | | GAGGGACAGCCGCCCTGGGTTGCCTTGTGAAAGATTATTTCCC |
| | | | TGAGCCAGTTACCGTAAGTTGGAACAGTGGGGCGCTGACAAG |
| | | | TGGTGTGCACACGTTTCCTGCCGTCCTGCAATCATCGGGCTTGT |
| | | | ATAGCCTCAGCTCTGTGGTCACTGTCCCAAGTTCATCGCTGGG |
| | | | CACTCAGACGTATATTTGCAATGTGAACCACAAACCTTCAAAT |
| | | | ACAAAAGTGGATAAACGCGTAGAACCGAAATCGTGTGATAAA |
| | | | ACTCACACATGCCCGCCATGCCCGGCACCTGAACTGCTTGGTG |
| | | | GTCCCAGCGTGTTCCTGTTCCCGCCGAAGCCTAAAGATACTCT |
| | | | AATGATCAGCCGTACGCCAGAGGTGACATGTGTCGTGGTTGAC |
| | | | GTGTCCCACGAAGATCCCGAAGTTAAGTTCAATTGGTATGTTG |
| | | | ATGGTGTAGAGGTACACAATGCTAAGACTAAACCTCGCGAGG |
| | | | AGCAGTACGCCTCGACCTATCGTGTCGTGAGCGTTCTGACCGT |
| | | | CCTTCACCAAGATTGGCTTAACGGCAAAGAATATAAGTGCAA |
| | | | GGTAAGCAATAAAGCACTTCCGGCCCCAATCGAGAAAACCAT |
| | | | TTCCAAGGCCAAAGGTCAACCAAGAGAACCCCAGGTGTATAC |
| | | | TCTTCCGCCTTCTCGTGAGGAAATGACTAAAAATCAAGTATCC |
| | | | CTTACGTGTCTGGTTAAAGGTTTTTATCCTAGCGATATTGCTGT |
| | | | TGAATGGGAATCGAACGGTCAGCCGGAGAATAATTATAAAAC |
| | | | AACGCCACCCGTCCTGGATAGCGACGGCTCATTTTTTCTGTAT |
| | | | AGCAAACTGACTGTAGATAAATCACGGTGGCAGCAGGGCAAT |
| | | | GTATTCAGTTGCTCCGTTATGCATGAAGCGTTACATAATCACT |
| | | | ACACGCAGAAATCTCTTAGTCTTTCACCCGGT |
| 1423 | ATX-P-3360 | HC IgG1-Fc aa | QVQLQESGPGLVKPSGTLSLTCAVSGGSISINNWWSWVRQPPGK |
| | | | GLEWLGKIYHSGNTDYNPSLKSRVTISVDKSKNQFSLRLSSVTAA |
| | | | DTAVYYCASGSDYDFWSGYLDYWGQGTLVTVSSASTKGPSVFP |
| | | | LAPSSKSTSGGTAALGCLVKDYFPEPVTVSWNSGALTSGVHTFPA |
| | | | VLQSSGLYSLSSVVTVPSSSLGTQTYICNVNHKPSNTKVDKRVEP |
| | | | KSCDKTHTCPPCPAPELLGGPSVFLFPPKPKDTLMISRTPEVTCVV |
| | | | VDVSHEDPEVKFNWYVDGVEVHNAKTKPREEQYASTYRVVSVL |
| | | | TVLHQDWLNGKEYKCKVSNKALPAPIEKTISKAKGQPREPQVYT |
| | | | LPPSREEMTKNQVSLTCLVKGFYPSDIAVEWESNGQPENNYKTTP |
| | | | PVLDSDGSFFLYSKLTVDKSRWQQGNVFSCSVMHEALHNHYTQ |
| | | | KSLSLSPG |
| 1424 | ATX-P-3362 | HC IgG1-Fc aa | EVQLVESGGGLVQPGRSLRLSCAASGFTFDDYAMHWVRQAPGK |
| | | | GLEWVSGISWNSGSIAYADSVKGRFTISRDNAKNSLYLQMNSLR |
| | | | VEDTAFFYCARVAAAAGIPFFDYWGQGTLVTVSSASTKGPSVFPL |
| | | | APSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPK |
| | | | SCDKTHTCPPCPAPELLGGPSVFLFPPKPKDTLMISRTPEVTCVVV |
| | | | DVSHEDPEVKFNWYVDGVEVHNAKTKPREEQYASTYRVVSVLT |
| | | | VLHQDWLNGKEYKCKVSNKALPAPIEKTISKAKGQPREPQVYTL |
| | | | PPSREEMTKNQVSLTCLVKGFYPSDIAVEWESNGQPENNYKTTPP |
| | | | VLDSDGSFFLYSKLTVDKSRWQQGNVFSCSVMHEALHNHYTQK |
| | | | SLSLSPG |
| 1425 | ATX-P-3363 | HC IgG1-Fc aa | QVQLQESGPGLVKPSGTLTLTCAVSGGSINRDNWWSWVRQPPGK |
| | | | GLEWIGQIYQSGSTNYNPSLKSRVTISVDKSKNQFSLKLSSVTAAD |
| | | | TAVYYCARVYDWNYGGHFDYWGQGTLVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1426 | ATX-P-3365 | HC IgG1-Fc aa | QVQLQESGPGLVKPSGTLSLTCAVSGDSISSNNWWSWVRQPPGK |
| | | | GLEWIGQIYHSGHTNYNPSLKSRVTISVDMSKNQFSLKLTSVTAA |
| | | | DTAVYYCARGATEILGYFDLWGRGTLVTVSSASTKGPSVFPLAPS |
| | | | SKSTSGGTAALGCLVKDYFPEPVTVSWNSGALTSGVHTFPAVLQ |
| | | | SSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDV |
| | | | SHEDPEVKFNWYVDGVEVHNAKTKPREEQYASTYRVVSVLTVL |
| | | | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPG |
| 1427 | ATX-P-3367 | HC IgG1-Fc aa | QVQLQESGPGLVKPSETLSLTCTVSGGSVSSGSYYWSWIRQPPGK |
| | | | GLEWIGYIYKSGFTNYNPSLKSRVTISVDTSKNQFSLKLSSVTAAD |
| | | | TAVYYCAIGGRGYSYGPFDYWGQGTLVTVSSASTKGPSVFPLAP |
| | | | SSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1428 | ATX-P-3369 | HC IgG1-Fc aa | QVQLVQSGAEVKKPGASVKVSCKASGYTFTGYYLHWVRQAPGQ |
| | | | GLEWMGWINPNSGDTNYVQNFQGRVTMTRDTSISTAYMELSRL |
| | | | RSDDTAVYYCARSYVDTSSSAFDIWGQGTMVTVSSASTKGPSVF |
| | | | PLAPSSKSTSGGTAALGCLVKDYFPEPVTVSWNSGALTSGVHTFP |
| | | | AVLQSSGLYSLSSVVTVPSSSLGTQTYICNVNHKPSNTKVDKRVE |
| | | | PKSCDKTHTCPPCPAPELLGGPSVFLFPPKPKDTLMISRTPEVTCV |
| | | | VVDVSHEDPEVKFNWYVDGVEVHNAKTKPREEQYASTYRVVSV |
| | | | LTVLHQDWLNGKEYKCKVSNKALPAPIEKTISKAKGQPREPQVY |
| | | | TLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQ |
| | | | KSLSLSPG |
| 1429 | ATX-P-3370 | HC IgG1-Fc aa | QVQLQESGPGLVKPSETLSLTCTVSGGSISSYYWSWIRQPPGKGL |
| | | | EWIGYVYYSGYTKYNPSLKSRVTISVDTPKTQFSLKMSSVTAADT |
| | | | AVYYCASVESKWNYGPFDIWGQGTMVTVSSASTKGPSVFPLAPS |
| | | | SKSTSGGTAALGCLVKDYFPEPVTVSWNSGALTSGVHTFPAVLQ |
| | | | SSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDV |
| | | | SHEDPEVKFNWYVDGVEVHNAKTKPREEQYASTYRVVSVLTVL |
| | | | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPG |
| 1430 | ATX-P-3371 | HC IgG1-Fc aa | QVQLQESGPGLVKPSETLSLTCTVSGDSISSYYWSWIRQPPGKGL |
| | | | EWIGYIYYSGFTKYNPSLNSRVTISVDTSKSQFSLKLTSVTAADTA |
| | | | VYYCAMREPRGRYNWNYGPFDIWGQGTMVTVSSASTKGPSVFP |
| | | | LAPSSKSTSGGTAALGCLVKDYFPEPVTVSWNSGALTSGVHTFPA |
| | | | VLQSSGLYSLSSVVTVPSSSLGTQTYICNVNHKPSNTKVDKRVEP |
| | | | KSCDKTHTCPPCPAPELLGGPSVFLFPPKPKDTLMISRTPEVTCVV |
| | | | VDVSHEDPEVKFNWYVDGVEVHNAKTKPREEQYASTYRVVSVL |
| | | | TVLHQDWLNGKEYKCKVSNKALPAPIEKTISKAKGQPREPQVYT |
| | | | LPPSREEMTKNQVSLTCLVKGFYPSDIAVEWESNGQPENNYKTTP |
| | | | PVLDSDGSFFLYSKLTVDKSRWQQGNVFSCSVMHEALHNHYTQ |
| | | | KSLSLSPG |
| 1431 | ATX-P-3373 | HC IgG1-Fc aa | QVQLQESGPGLVKPSGTLSLTCAVSGGSISNDNWWSWVRQPPGK |
| | | | GLEWIGKIYHSEFTNYNPSLKSRVTISVDKSKNQFSLELNSVTAAD |
| | | | TAVYYCAREYHNFMDVWCKGTTVTVSSASTKGPSVFPLAPSSKS |
| | | | TSGGTAALGCLVKDYFPEPVTVSWNSGALTSGVHTFPAVLQSSG |
| | | | LYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVYTLPPSREE |
| | | | MTKNQVSLTCLVKGFYPSDIAVEWESNGQPENNYKTTPPVLDSD |
| | | | GSFFLYSKLTVDKSRWQQGNVFSCSVMHEALHNHYTQKSLSLSP |
| | | | G |
| 1432 | ATX-P-3375 | HC IgG1-Fc aa | QVQLQESGPGLVKPSETLSLTCTVSGDSVSSGKYFWSWIRQSPGK |
| | | | GLEWIGYIYYSGSTNYNPSLKNRVTISVDTSKKELSLKLSSVTAAD |
| | | | TAVYYCARGGHNWNYAAFDIWGQGTMVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1433 | ATX-P-3378 | HC IgG1-Fc aa | QVQLQESGPGLVKPSGTLSLTCAVSGGSISNDNWWSWVRQSPGK |
| | | | GLEWIGKIYHSEFTNYNPSLKSRVTISVDKSKNQFSLELSSVAAAD |
| | | | TAVYYCAREYENFMDVWGKGTTVTVSSASTKGPSVFPLAPSSKS |
| | | | TSGGTAALGCLVKDYFPEPVTVSWNSGALTSGVHTFPAVLQSSG |
| | | | LYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVYTLPPSREE |
| | | | MTKNQVSLTCLVKGFYPSDIAVEWESNGQPENNYKTTPPVLDSD |
| | | | GSFFLYSKLTVDKSRWQQGNVFSCSVMHEALHNHYTQKSLSLSP |
| | | | G |
| 1434 | ATX-P-3382 | HC IgG1-Fc aa | QVQLQESGPGLVKPSGTLSLTCAVSGDSISSNSWWNWVRQPPGK |
| | | | GLEWIGQIFHSGHTNYNPSLKSRVTMSIDKSKNQFSLKLTSVTAA |
| | | | DTALYYCARGGEYENSANWFDPWGQGTLVTVSSASTKGPSVFPL |
| | | | APSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPK |
| | | | SCDKTHTCPPCPAPELLGGPSVFLFPPKPKDTLMISRTPEVTCVVV |
| | | | DVSHEDPEVKFNWYVDGVEVHNAKTKPREEQYASTYRVVSVLT |
| | | | VLHQDWLNGKEYKCKVSNKALPAPIEKTISKAKGQPREPQVYTL |
| | | | PPSREEMTKNQVSLTCLVKGFYPSDIAVEWESNGQPENNYKTTPP |
| | | | VLDSDGSFFLYSKLTVDKSRWQQGNVFSCSVMHEALHNHYTQK |
| | | | SLSLSPG |
| 1435 | ATX-P-3383 | HC IgG1-Fc aa | QVQLQESGPGLVKPSGTLSLTCAVSGVSINKDNWWTWVRQPPG |
| | | | KGLEWIGKIYHSGSTNYNPSLKSRVTISVDNSKNQFSLKLNSVTA |
| | | | ADTAVYYCTRGGDIAMEGDWFAPWGQGTLVTVSSASTKGPSVF |
| | | | PLAPSSKSTSGGTAALGCLVKDYFPEPVTVSWNSGALTSGVHTFP |
| | | | AVLQSSGLYSLSSVVTVPSSSLGTQTYICNVNHKPSNTKVDKRVE |
| | | | PKSCDKTHTCPPCPAPELLGGPSVFLFPPKPKDTLMISRTPEVTCV |
| | | | VVDVSHEDPEVKFNWYVDGVEVHNAKTKPREEQYASTYRVVSV |
| | | | LTVLHQDWLNGKEYKCKVSNKALPAPIEKTISKAKGQPREPQVY |
| | | | TLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQ |
| | | | KSLSLSPG |
| 1436 | ATX-P-3384 | HC IgG1-Fc aa | QVQLQESGPGLVKPSGTLSLTCAVSGGSISSDNWWNWVRQPPGK |
| | | | GLEWLGKIYHSGNTDYNPSLKSRVTISVDKSKNQFSLRLSSVTAA |
| | | | DTAVYYCARGGTETTGYMDVWGKGTTVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1437 | ATX-P-3386 | HC IgG1-Fc aa | QVQLQESGPGLVKPSGTLSLTCAVSGGSISNDNWWSWVRQPPGK |
| | | | GLEWIGKIYHSGFTNYNPSLKSRVTISVDKSKNQFSLKLNSVTAA |
| | | | DTAVYYCARGGDNFMEGEGLGTWGPGTLVTVSSASTKGPSVFPL |
| | | | APSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPK |
| | | | SCDKTHTCPPCPAPELLGGPSVFLFPPKPKDTLMISRTPEVTCVVV |
| | | | DVSHEDPEVKFNWYVDGVEVHNAKTKPREEQYASTYRVVSVLT |
| | | | VLHQDWLNGKEYKCKVSNKALPAPIEKTISKAKGQPREPQVYTL |
| | | | PPSREEMTKNQVSLTCLVKGFYPSDIAVEWESNGQPENNYKTTPP |
| | | | VLDSDGSFFLYSKLTVDKSRWQQGNVFSCSVMHEALHNHYTQK |
| | | | SLSLSPG |
| 1438 | ATX-P-3387 | HC IgG1-Fc aa | QVQLQESGPGLVKPSGTLSLTCAVSGGSISNDNWWNWVRQSPGK |
| | | | GLEWIGKIYHSGFTDYNPSLKSRVTISVDKSKNQFSLKLSSVTAAD |
| | | | TAVYYCARGNHNYLWIGYLGTWGPGTLVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1439 | ATX-P-3389 | HC IgG1-Fc aa | QVQLQESGPGLVKPSGTLSLICAVSGGSISSSNWWSWVRQPPGKG |
| | | | LEWIGRIYHSGHTNYNPSLKSRVTISVDKSKNQFSLKLTSVTAAD |
| | | | TAVYYCARGRDLTGYFDYWGQGTLVTVSSASTKGPSVFPLAPSS |
| | | | KSTSGGTAALGCLVKDYFPEPVTVSWNSGALTSGVHTFPAVLQS |
| | | | SGLYSLSSVVTVPSSSLGTQTYICNVNHKPSNTKVDKRVEPKSCD |
| | | | KTHTCPPCPAPELLGGPSVFLFPPKPKDTLMISRTPEVTCVVVDVS |
| | | | HEDPEVKFNWYVDGVEVHNAKTKPREEQYASTYRVVSVLTVLH |
| | | | QDWLNGKEYKCKVSNKALPAPIEKTISKAKGQPREPQVYTLPPSR |
| | | | EEMTKNQVSLTCLVKGFYPSDIAVEWESNGQPENNYKTTPPVLD |
| | | | SDGSFFLYSKLTVDKSRWQQGNVFSCSVMHEALHNHYTQKSLSL |
| | | | SPG |
| 1440 | ATX-P-3391 | HC IgG1-Fc aa | QVQLQESGPGLVKPSGTLSLTCAVSGGSISNDNWWSWVRQSPGK |
| | | | GLEWIGKIYHSEFTNYNPSLKSRVTISVDKSKNQFSLELSSVTAAD |
| | | | TAVYYCARGVDFWSGSHMDVWGKGTTVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1441 | ATX-P-3392 | HC IgG1-Fc aa | QVQLQESGPGLVKPSGTLSLTCAVSGGSISNDNWWSWVRQSPGK |
| | | | GLEWIGKIYHSEFTNYNPSLKSRVTISVDKSKNQFSLELSSVTAAD |
| | | | TAVYFCAREGLELPHMDVWGKGTTVTVSSASTKGPSVFPLAPSS |
| | | | KSTSGGTAALGCLVKDYFPEPVTVSWNSGALTSGVHTFPAVLQS |
| | | | SGLYSLSSVVTVPSSSLGTQTYICNVNHKPSNTKVDKRVEPKSCD |
| | | | KTHTCPPCPAPELLGGPSVFLFPPKPKDTLMISRTPEVTCVVVDVS |
| | | | HEDPEVKFNWYVDGVEVHNAKTKPREEQYASTYRVVSVLTVLH |
| | | | QDWLNGKEYKCKVSNKALPAPIEKTISKAKGQPREPQVYTLPPSR |
| | | | EEMTKNQVSLTCLVKGFYPSDIAVEWESNGQPENNYKTTPPVLD |
| | | | SDGSFFLYSKLTVDKSRWQQGNVFSCSVMHEALHNHYTQKSLSL |
| | | | SPG |
| 1442 | ATX-P-3395 | HC IgG1-Fc aa | EVQLVESGGGVVKPGGSLRLSCAASGFTFSSYWMHWVRQAPGK |
| | | | GLVWVSRINSDGSSTSYADSVQGRFTISRDIAKNTLYLQMNSLRA |
| | | | EDTAVYYCARGQHLVQDYWGQGTLVTVSSASTKGPSVFPLAPSS |
| | | | KSTSGGTAALGCLVKDYFPEPVTVSWNSGALTSGVHTFPAVLQS |
| | | | SGLYSLSSVVTVPSSSLGTQTYICNVNHKPSNTKVDKRVEPKSCD |
| | | | KTHTCPPCPAPELLGGPSVFLFPPKPKDTLMISRTPEVTCVVVDVS |
| | | | HEDPEVKFNWYVDGVEVHNAKTKPREEQYASTYRVVSVLTVLH |
| | | | QDWLNGKEYKCKVSNKALPAPIEKTISKAKGQPREPQVYTLPPSR |
| | | | EEMTKNQVSLTCLVKGFYPSDIAVEWESNGQPENNYKTTPPVLD |
| | | | SDGSFFLYSKLTVDKSRWQQGNVFSCSVMHEALHNHYTQKSLSL |
| | | | SPG |
| 1443 | ATX-P-3396 | HC IgG1-Fc aa | QVQLVQSGAEVKNPGASVKVSCKASGYTFTGYYMHWVRQAPG |
| | | | QGLEWMGWINPNSGGTNYAQKFQGRVTMTRDTSISTAYMELSR |
| | | | LRSDDTAVYYCARERGDYDGDAFDIWGQGTMVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKR |
| | | | VEPKSCDKTHTCPPCPAPELLGGPSVFLFPPKPKDTLMISRTPEVT |
| | | | CVVVDVSHEDPEVKFNWYVDGVEVHNAKTKPREEQYASTYRVV |
| | | | SVLTVLHQDWLNGKEYKCKVSNKALPAPIEKTISKAKGQPREPQ |
| | | | VYTLPPSREEMTKNQVSLTCLVKGFYPSDIAVEWESNGQPENNY |
| | | | KTTPPVLDSDGSFFLYSKLTVDKSRWQQGNVFSCSVMHEALHNH |
| | | | YTQKSLSLSPG |
| 1444 | ATX-P-3397 | HC IgG1-Fc aa | EVQLVESGGGLVQPGGSLRLSCAASGFTFSNYYMNWVRQAPGK |
| | | | GLEWVANIKQDGSEKYYVDSVKGRFTISRDNAKNSLYLQMNSLR |
| | | | AEDTAVYYCARDGYYNFDAFDIWGQGTMVTVSSASTKGPSVFPL |
| | | | APSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPK |
| | | | SCDKTHTCPPCPAPELLGGPSVFLFPPKPKDTLMISRTPEVTCVVV |
| | | | DVSHEDPEVKFNWYVDGVEVHNAKTKPREEQYASTYRVVSVLT |
| | | | VLHQDWLNGKEYKCKVSNKALPAPIEKTISKAKGQPREPQVYTL |
| | | | PPSREEMTKNQVSLTCLVKGFYPSDIAVEWESNGQPENNYKTTPP |
| | | | VLDSDGSFFLYSKLTVDKSRWQQGNVFSCSVMHEALHNHYTQK |
| | | | SLSLSPG |
| 1445 | ATX-P-3405 | HC IgG1-Fc aa | EVQLVESGGGLVQPGGSLRLSCAASGFTFSSYSMNWVRQAPGKG |
| | | | LEWVSSISSSSSYIYYADSVKGRFTISRDNAKNSLYLQMNSLRAED |
| | | | TAVYYCAREGYDNWNSACDIWGQGTMVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1446 | ATX-P-3406 | HC IgG1-Fc aa | QITLKESGPTLVKPTQTLTLTCTFSGFSLSTSGVGVGWIRQPPGKA |
| | | | LEWLALIYWNGDERYSPSLKSRLTITKDTSKNQVVLTMTNMDPV |
| | | | DTATYYCAHSPNWGYEAFDIWGQGTMVTVSSASTKGPSVFPLAP |
| | | | SSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1447 | ATX-P-3407 | HC IgG1-Fc aa | EVQLVESGGGLVPPGGSLRLSCAASGFTFSSYCMNWVRQAPGKG |
| | | | LEWVSSINSDSSYIYYADSVKGRFTISRDNAKNSLYLQMNSLRAE |
| | | | DTAVYYCAREGYYNWNGGNDYWGQGTLVTVSSASTKGPSVFPL |
| | | | APSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPK |
| | | | SCDKTHTCPPCPAPELLGGPSVFLFPPKPKDTLMISRTPEVTCVVV |
| | | | DVSHEDPEVKFNWYVDGVEVHNAKTKPREEQYASTYRVVSVLT |
| | | | VLHQDWLNGKEYKCKVSNKALPAPIEKTISKAKGQPREPQVYTL |
| | | | PPSREEMTKNQVSLTCLVKGFYPSDIAVEWESNGQPENNYKTTPP |
| | | | VLDSDGSFFLYSKLTVDKSRWQQGNVFSCSVMHEALHNHYTQK |
| | | | SLSLSPG |
| 1448 | ATX-P-3408 | HC IgG1-Fc aa | EVQLVESGGGLVQPGGSLRLSCAASGFTFSSYWMHWVRQAPGK |
| | | | GLVWVSHINSDGSSTNYADSVKGRFTISRDNAKNTLYLQMNSLR |
| | | | SEDTAVYYCAREGYWNGDTFDIWGQGTMVTVSSASTKGPSVFPL |
| | | | APSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPK |
| | | | SCDKTHTCPPCPAPELLGGPSVFLFPPKPKDTLMISRTPEVTCVVV |
| | | | DVSHEDPEVKFNWYVDGVEVHNAKTKPREEQYASTYRVVSVLT |
| | | | VLHQDWLNGKEYKCKVSNKALPAPIEKTISKAKGQPREPQVYTL |
| | | | PPSREEMTKNQVSLTCLVKGFYPSDIAVEWESNGQPENNYKTTPP |
| | | | VLDSDGSFFLYSKLTVDKSRWQQGNVFSCSVMHEALHNHYTQK |
| | | | SLSLSPG |
| 1449 | ATX-P-3410 | HC IgG1-Fc aa | EVQLVESGGGLVQPGGSLRLSCAASGFTFSAYDMHWVRQTPGK |
| | | | GLEWVSTIGTAGDTYYPDSVKGRFTISRDFARRSLYLHMNNLRA |
| | | | GDTAVYYCARGGSSSWYDWYFDLWGRGTLVTVSSASTKGPSVF |
| | | | PLAPSSKSTSGGTAALGCLVKDYFPEPVTVSWNSGALTSGVHTFP |
| | | | AVLQSSGLYSLSSVVTVPSSSLGTQTYICNVNHKPSNTKVDKRVE |
| | | | PKSCDKTHTCPPCPAPELLGGPSVFLFPPKPKDTLMISRTPEVTCV |
| | | | VVDVSHEDPEVKFNWYVDGVEVHNAKTKPREEQYASTYRVVSV |
| | | | LTVLHQDWLNGKEYKCKVSNKALPAPIEKTISKAKGQPREPQVY |
| | | | TLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQ |
| | | | KSLSLSPG |
| 1450 | ATX-P-3412 | HC IgG1-Fc aa | QVQLQESGPGLVKPSGTLSLTCAVSGGSISSSTWWSWVRQPPGK |
| | | | GLEWIGEIYHSGSTNYNPSLKSRVTISLDKSKNQFSLKLSSVTAAD |
| | | | TAVYYCAISLYNWNDAAFDIWGQGTMVTVSSASTKGPSVFPLAP |
| | | | SSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1451 | ATX-P-3413 | HC IgG1-Fc aa | QVQLVQSGSEVKKPGASVKVSCKTSGYTFTGYYMHWVRQAPGQ |
| | | | GLEWMGWINPNSGGTTYAQKFQGRVTMTRDTSISTAYMELNRL |
| | | | RSDDTAVYYCAREAGGSGEYFQHWGQGTPVTVSSASTKGPSVFP |
| | | | LAPSSKSTSGGTAALGCLVKDYFPEPVTVSWNSGALTSGVHTFPA |
| | | | VLQSSGLYSLSSVVTVPSSSLGTQTYICNVNHKPSNTKVDKRVEP |
| | | | KSCDKTHTCPPCPAPELLGGPSVFLFPPKPKDTLMISRTPEVTCVV |
| | | | VDVSHEDPEVKFNWYVDGVEVHNAKTKPREEQYASTYRVVSVL |
| | | | TVLHQDWLNGKEYKCKVSNKALPAPIEKTISKAKGQPREPQVYT |
| | | | LPPSREEMTKNQVSLTCLVKGFYPSDIAVEWESNGQPENNYKTTP |
| | | | PVLDSDGSFFLYSKLTVDKSRWQQGNVFSCSVMHEALHNHYTQ |
| | | | KSLSLSPG |
| 1452 | ATX-P-3414 | HC IgG1-Fc aa | QITLKESGPTLVKPTQTLTLTCTFSGFSLSTSGVGVGWIRQPPGKA |
| | | | LEWLALIYWNDVKFYSPSLQSRLTITKDTSKNQVVLTLTNMDPV |
| | | | DTATYYCAHGDNWNYRAFDIWGQGTMVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1453 | ATX-P-3419 | HC IgG1-Fc aa | QVQLVQSGAEVKKPGASVKVSCKASGYTFTSYGISWVRQAPGQ |
| | | | GLEWMGWISAYNGNTHYAQNLQGRVTMTTDTSTSTAYMELRSL |
| | | | RSDDTAVYYCARSIAALDFWGQGTPVTVSSASTKGPSVFPLAPSS |
| | | | KSTSGGTAALGCLVKDYFPEPVTVSWNSGALTSGVHTFPAVLQS |
| | | | SGLYSLSSVVTVPSSSLGTQTYICNVNHKPSNTKVDKRVEPKSCD |
| | | | KTHTCPPCPAPELLGGPSVFLFPPKPKDTLMISRTPEVTCVVVDVS |
| | | | HEDPEVKFNWYVDGVEVHNAKTKPREEQYASTYRVVSVLTVLH |
| | | | QDWLNGKEYKCKVSNKALPAPIEKTISKAKGQPREPQVYTLPPSR |
| | | | EEMTKNQVSLTCLVKGFYPSDIAVEWESNGQPENNYKTTPPVLD |
| | | | SDGSFFLYSKLTVDKSRWQQGNVFSCSVMHEALHNHYTQKSLSL |
| | | | SPG |
| 1454 | ATX-P-3421 | HC IgG1-Fc aa | QVQLQESGPGLVKPSGTLSLTCAVSGGSISSNNWWSWVRQPPGK |
| | | | GLEWIGQIYHSENTNYNPSLKSRVTISVDKSKNQFSLKLSSVTAA |
| | | | DTAIYYCARVDDWNYGGHFDYWGQGTLVTVSSASTKGPSVFPL |
| | | | APSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPK |
| | | | SCDKTHTCPPCPAPELLGGPSVFLFPPKPKDTLMISRTPEVTCVVV |
| | | | DVSHEDPEVKFNWYVDGVEVHNAKTKPREEQYASTYRVVSVLT |
| | | | VLHQDWLNGKEYKCKVSNKALPAPIEKTISKAKGQPREPQVYTL |
| | | | PPSREEMTKNQVSLTCLVKGFYPSDIAVEWESNGQPENNYKTTPP |
| | | | VLDSDGSFFLYSKLTVDKSRWQQGNVFSCSVMHEALHNHYTQK |
| | | | SLSLSPG |
| 1455 | ATX-P-3428 | HC IgG1-Fc aa | QVQLVQSGAEVKKPGASVKVSCKASGYTFTGYGISWVRQAPGQ |
| | | | GLEWMGWISAYNGNTHYAQNLQGRVTMTTDTSINTAYMELRS |
| | | | LISDDTAFYYCARAGLGIDYWGQGTLVTVSSASTKGPSVFPLAPS |
| | | | SKSTSGGTAALGCLVKDYFPEPVTVSWNSGALTSGVHTFPAVLQ |
| | | | SSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDV |
| | | | SHEDPEVKFNWYVDGVEVHNAKTKPREEQYASTYRVVSVLTVL |
| | | | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPG |
| 1456 | ATX-P-3430 | HC IgG1-Fc aa | QVQLQESGPGLVKPSGTLSLTCAVSGGSISSSDWWSWVRQSPGK |
| | | | GLEWIGEIYHSGSTNYNPSLKSRVSISVDKSKNQFSLKLSSVTAAD |
| | | | TAVYYCARGGHIGYYYMDVWGKGTTVTVSSASTKGPSVFPLAPS |
| | | | SKSTSGGTAALGCLVKDYFPEPVTVSWNSGALTSGVHTFPAVLQ |
| | | | SSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDV |
| | | | SHEDPEVKFNWYVDGVEVHNAKTKPREEQYASTYRVVSVLTVL |
| | | | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPG |
| 1457 | ATX-P-3431 | HC IgG1-Fc aa | EVQLVQSGAEVKKPGESLKISCKGSGYSFTNYWIGWVRQMPGKG |
| | | | LEWMGIYPGDSDTIYSPSFQGQVTFSADKSINTAYLQWSSLKASD |
| | | | TAMYYCARGRGYSGYEGAFDIWGQGTMVTVSSASTKGPSVFPL |
| | | | APSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPK |
| | | | SCDKTHTCPPCPAPELLGGPSVFLFPPKPKDTLMISRTPEVTCVVV |
| | | | DVSHEDPEVKFNWYVDGVEVHNAKTKPREEQYASTYRVVSVLT |
| | | | VLHQDWLNGKEYKCKVSNKALPAPIEKTISKAKGQPREPQVYTL |
| | | | PPSREEMTKNQVSLTCLVKGFYPSDIAVEWESNGQPENNYKTTPP |
| | | | VLDSDGSFFLYSKLTVDKSRWQQGNVFSCSVMHEALHNHYTQK |
| | | | SLSLSPG |
| 1458 | ATX-P-3435 | HC IgG1-Fc aa | QVQLVQSGAEVKKPGASVKVSCKASGYTFTNYGISWVRQAPGQ |
| | | | GLEWMGWISAYNGNTHYAQKLQGRVTMTTDTSTSTAYMELRSL |
| | | | RSDDTAVYYCARAAAAVDYWGQGTLVTVSSASTKGPSVFPLAPS |
| | | | SKSTSGGTAALGCLVKDYFPEPVTVSWNSGALTSGVHTFPAVLQ |
| | | | SSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDV |
| | | | SHEDPEVKFNWYVDGVEVHNAKTKPREEQYASTYRVVSVLTVL |
| | | | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPG |
| 1459 | ATX-P-3436 | HC IgG1-Fc aa | QVQLQESGPGLVKPSETLSLSCTVSGGSVSSGSYYWSWIRQPPGK |
| | | | GLEWIGYIYYSGSTKYNPSLKSRVTISVDTSKNQVSLKLSSVTAA |
| | | | DTAVYYCARDKGHWNYGAFDIWGQGTMVTVSSASTKGPSVFPL |
| | | | APSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPK |
| | | | SCDKTHTCPPCPAPELLGGPSVFLFPPKPKDTLMISRTPEVTCVVV |
| | | | DVSHEDPEVKFNWYVDGVEVHNAKTKPREEQYASTYRVVSVLT |
| | | | VLHQDWLNGKEYKCKVSNKALPAPIEKTISKAKGQPREPQVYTL |
| | | | PPSREEMTKNQVSLTCLVKGFYPSDIAVEWESNGQPENNYKTTPP |
| | | | VLDSDGSFFLYSKLTVDKSRWQQGNVFSCSVMHEALHNHYTQK |
| | | | SLSLSPG |
| 1460 | ATX-P-3437 | HC IgG1-Fc aa | QVQLQESGPGLVKPSGTLSLTCAVSGGSISSNNWWSWVRQPPGK |
| | | | GLEWIGEIYHSGNINYNPSLKSRVTISVDKSKNQFSLKLSSVTAAD |
| | | | TAVYYCARGGEYGDPVGYFDLWGRGTLVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1461 | ATX-P-3440 | HC IgG1-Fc aa | QVQLVQSGAEVKKPGASVKVSCKASGYTFTTYGISWVRQAPGQ |
| | | | GLEWMGWISAYSGNTNYAQKLQGRVTMTTDTSTSTAYMELRSL |
| | | | RSDDTAVYYCARERVSNWVFDFWGQGTLVTVSSASTKGPSVFPL |
| | | | APSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPK |
| | | | SCDKTHTCPPCPAPELLGGPSVFLFPPKPKDTLMISRTPEVTCVVV |
| | | | DVSHEDPEVKFNWYVDGVEVHNAKTKPREEQYASTYRVVSVLT |
| | | | VLHQDWLNGKEYKCKVSNKALPAPIEKTISKAKGQPREPQVYTL |
| | | | PPSREEMTKNQVSLTCLVKGFYPSDIAVEWESNGQPENNYKTTPP |
| | | | VLDSDGSFFLYSKLTVDKSRWQQGNVFSCSVMHEALHNHYTQK |
| | | | SLSLSPG |
| 1462 | ATX-P-3442 | HC IgG1-Fc aa | QVQLQESGPGLVKPSGTLSLTCAVSGGSISSSDWWSWVRQPPGK |
| | | | GLEWIGEIYHSRNTNYNPSLKSRVTISVDKSKNQFSLKLSSVTAAD |
| | | | TAVYYCASGGVVADYYMDVWGKGTTVTVSSASTKGPSVFPLAP |
| | | | SSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1463 | ATX-P-3444 | HC IgG1-Fc aa | EVQLVESGGGLVKPGGSLRLSCAASGFTFSSYGMNWVRQAPGK |
| | | | GLEWVSSISGYGSYIYYAASVKGRFTISRDSAKNSLYLQMNSLSA |
| | | | EDTAVYYCARELGGIYYGVGYFDLWGRGTLVTVSSASTKGPSVF |
| | | | PLAPSSKSTSGGTAALGCLVKDYFPEPVTVSWNSGALTSGVHTFP |
| | | | AVLQSSGLYSLSSVVTVPSSSLGTQTYICNVNHKPSNTKVDKRVE |
| | | | PKSCDKTHTCPPCPAPELLGGPSVFLFPPKPKDTLMISRTPEVTCV |
| | | | VVDVSHEDPEVKFNWYVDGVEVHNAKTKPREEQYASTYRVVSV |
| | | | LTVLHQDWLNGKEYKCKVSNKALPAPIEKTISKAKGQPREPQVY |
| | | | TLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQ |
| | | | KSLSLSPG |
| 1464 | ATX-P-3447 | HC IgG1-Fc aa | QVQLQESGPGLVKPSETLSLTCAVSGGSVSSGNYYWSWIRQPPG |
| | | | KGLEWIGYIYSSGSTNYNPSLKSRVTISIDTSKNQFSLKLSSVTAA |
| | | | DTAVYYCARSPRGYSYGPFAYWGQGTLVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1465 | ATX-P-3448 | HC IgG1-Fc aa | QVQLQESGPGLVKPSQTLSLTCTVSGGSISSGGYNWNWIRQHPGK |
| | | | GLEWIGYINYSGNTYYNPSLKSRVTISVDTSKNQFSLKLSSVTAA |
| | | | DTAVYYCARGPLDTAMAPFDYWGQGTLVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1466 | ATX-P-3449 | HC IgG1-Fc aa | QVQLQESGPGLVKPSETLSLTCTVSGDSISSYYWSWIRQPPGKGL |
| | | | EWIGYIYYSGFTKYNPSLKSRVTISVDTSKNQFSLRLSSVTAADTA |
| | | | VYYCASVDSKWNYGSFHIWGQGTMVTVSSASTKGPSVFPLAPSS |
| | | | KSTSGGTAALGCLVKDYFPEPVTVSWNSGALTSGVHTFPAVLQS |
| | | | SGLYSLSSVVTVPSSSLGTQTYICNVNHKPSNTKVDKRVEPKSCD |
| | | | KTHTCPPCPAPELLGGPSVFLFPPKPKDTLMISRTPEVTCVVVDVS |
| | | | HEDPEVKFNWYVDGVEVHNAKTKPREEQYASTYRVVSVLTVLH |
| | | | QDWLNGKEYKCKVSNKALPAPIEKTISKAKGQPREPQVYTLPPSR |
| | | | EEMTKNQVSLTCLVKGFYPSDIAVEWESNGQPENNYKTTPPVLD |
| | | | SDGSFFLYSKLTVDKSRWQQGNVFSCSVMHEALHNHYTQKSLSL |
| | | | SPG |
| 1467 | ATX-P-3450 | HC IgG1-Fc aa | QVQLQESGPGLVKPSETLSLTCSVSGDSVSNNRYYWSWIRQPPGK |
| | | | GLEWIGYIYYSGSTNYNPSLKSRVTISVDTSKNQLSLNLSSVTAAD |
| | | | TAVYYCARDRPSRYKWNYDAFAIWGQGTMVTVSSASTKGPSVF |
| | | | PLAPSSKSTSGGTAALGCLVKDYFPEPVTVSWNSGALTSGVHTFP |
| | | | AVLQSSGLYSLSSVVTVPSSSLGTQTYICNVNHKPSNTKVDKRVE |
| | | | PKSCDKTHTCPPCPAPELLGGPSVFLFPPKPKDTLMISRTPEVTCV |
| | | | VVDVSHEDPEVKFNWYVDGVEVHNAKTKPREEQYASTYRVVSV |
| | | | LTVLHQDWLNGKEYKCKVSNKALPAPIEKTISKAKGQPREPQVY |
| | | | TLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQ |
| | | | KSLSLSPG |
| 1468 | ATX-P-3451 | HC IgG1-Fc aa | QVQLQQWGAGLLKPSETLSLTCAVYGGSFSDYYWTWIRQPPGK |
| | | | GLEWIGEVNHIGSTNYNPSLKSRVTISVDTSKNQFSLNLSSVTAAD |
| | | | TAVYYCAKYSTSWYGWFDPWGQGTLVTVSSASTKGPSVFPLAPS |
| | | | SKSTSGGTAALGCLVKDYFPEPVTVSWNSGALTSGVHTFPAVLQ |
| | | | SSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDV |
| | | | SHEDPEVKFNWYVDGVEVHNAKTKPREEQYASTYRVVSVLTVL |
| | | | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPG |
| 1469 | ATX-P-3453 | HC IgG1-Fc aa | QVQLQESGPGLVKPSGTLSLTCAVSGDSISSNNWWSWFRQPPGK |
| | | | GLEWIGRIYHSGTTNYNPSLESRVTISVDNSRNQFSLKLSSVTAAD |
| | | | TAVYYCARGEEWELRGGVFDIWGQGTMVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1470 | ATX-P-3454 | HC IgG1-Fc aa | QVQLVQSGPELKKPGASVKVSCKASGYTFTNYAVNWVRQAPGQ |
| | | | GLEWMGWINTNTGHPTYAQGFTGRFVFSLDTSVSTAYLQISSLK |
| | | | AEDTAVYYCARDSSSWGYWGQGTLVTVSSASTKGPSVFPLAPSS |
| | | | KSTSGGTAALGCLVKDYFPEPVTVSWNSGALTSGVHTFPAVLQS |
| | | | SGLYSLSSVVTVPSSSLGTQTYICNVNHKPSNTKVDKRVEPKSCD |
| | | | KTHTCPPCPAPELLGGPSVFLFPPKPKDTLMISRTPEVTCVVVDVS |
| | | | HEDPEVKFNWYVDGVEVHNAKTKPREEQYASTYRVVSVLTVLH |
| | | | QDWLNGKEYKCKVSNKALPAPIEKTISKAKGQPREPQVYTLPPSR |
| | | | EEMTKNQVSLTCLVKGFYPSDIAVEWESNGQPENNYKTTPPVLD |
| | | | SDGSFFLYSKLTVDKSRWQQGNVFSCSVMHEALHNHYTQKSLSL |
| | | | SPG |
| 1471 | ATX-P-3455 | HC IgG1-Fc aa | QVQLQESGPGLVTPSGTLSLTCAVSGDSISSDNWWSWVRQPPGK |
| | | | GLEWIGEIYHSENTNYNPSLKSRVTISVDKSKNQFSLRLNSVTAA |
| | | | DTAMYYCARGRELTGLFDYWGQGTLVTVSSASTKGPSVFPLAPS |
| | | | SKSTSGGTAALGCLVKDYFPEPVTVSWNSGALTSGVHTFPAVLQ |
| | | | SSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDV |
| | | | SHEDPEVKFNWYVDGVEVHNAKTKPREEQYASTYRVVSVLTVL |
| | | | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPG |
| 1472 | ATX-P-3456 | HC IgG1-Fc aa | QVQLQESGPGLVKPSGTLSLTCAVSGGSISSNNWWSWVRQPPGK |
| | | | GLEWIGDIYHSGNTNYNPSLKSRVTLSVDMSKNQFSLKLNSVTA |
| | | | ADTAVYYCARGGIAVTAYFDYWGQGTLVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1473 | ATX-P-3458 | HC IgG1-Fc aa | QVQLVQSGAEVKKPGASVKVSCKASGYTFTGYYLHWVRQAPGQ |
| | | | GLEWMGWINPHSGGTIYAQKFQDRVTMTRDTSISTAYMELSRLR |
| | | | SDDTAVYYCANWNSGDSYYYYMDVWGKGTTVTVSSASTKGPS |
| | | | VFPLAPSSKSTSGGTAALGCLVKDYFPEPVTVSWNSGALTSGVHT |
| | | | FPAVLQSSGLYSLSSVVTVPSSSLGTQTYICNVNHKPSNTKVDKR |
| | | | VEPKSCDKTHTCPPCPAPELLGGPSVFLFPPKPKDTLMISRTPEVT |
| | | | CVVVDVSHEDPEVKFNWYVDGVEVHNAKTKPREEQYASTYRVV |
| | | | SVLTVLHQDWLNGKEYKCKVSNKALPAPIEKTISKAKGQPREPQ |
| | | | VYTLPPSREEMTKNQVSLTCLVKGFYPSDIAVEWESNGQPENNY |
| | | | KTTPPVLDSDGSFFLYSKLTVDKSRWQQGNVFSCSVMHEALHNH |
| | | | YTQKSLSLSPG |
| 1474 | ATX-P-3459 | HC IgG1-Fc aa | QVQLQESGPGLVKPSGTLSLTCAVSGGSISSDNWWNWVRQPPGK |
| | | | GLEWIGKIYHSGNTNYNPSLKSRVTISVDESKNQFSLRLYSVTAA |
| | | | DTAVYYCARGVDFWSGSHMDVWGKGTTVTVSSASTKGPSVFPL |
| | | | APSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPK |
| | | | SCDKTHTCPPCPAPELLGGPSVFLFPPKPKDTLMISRTPEVTCVVV |
| | | | DVSHEDPEVKFNWYVDGVEVHNAKTKPREEQYASTYRVVSVLT |
| | | | VLHQDWLNGKEYKCKVSNKALPAPIEKTISKAKGQPREPQVYTL |
| | | | PPSREEMTKNQVSLTCLVKGFYPSDIAVEWESNGQPENNYKTTPP |
| | | | VLDSDGSFFLYSKLTVDKSRWQQGNVFSCSVMHEALHNHYTQK |
| | | | SLSLSPG |
| 1475 | ATX-P-3462 | HC IgG1-Fc aa | QVQLQESGPGLVKPSGTLSLTCAVSGGSISSNNWWSWVRQAPGK |
| | | | GLEWIGKIYQSGSTMYNPSLKSRVTMSVDKSKNQFSLILSSVTAA |
| | | | DTAVYYCARDKPPLLGAFDYWGQGTLVTVSSASTKGPSVFPLAP |
| | | | SSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1476 | ATX-P-3463 | HC IgG1-Fc aa | QVQLVESGGGVVQPGRSLRLSCAASGFTFSSYAMHWVRQAPGK |
| | | | GLEWVAVISYDGSNKYYADSVKGRFTISRDNSKNTLSLQMNSLR |
| | | | AEDTAVYYCAKRGGYSYGPFEHWGQGTLVTVSSASTKGPSVFPL |
| | | | APSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPK |
| | | | SCDKTHTCPPCPAPELLGGPSVFLFPPKPKDTLMISRTPEVTCVVV |
| | | | DVSHEDPEVKFNWYVDGVEVHNAKTKPREEQYASTYRVVSVLT |
| | | | VLHQDWLNGKEYKCKVSNKALPAPIEKTISKAKGQPREPQVYTL |
| | | | PPSREEMTKNQVSLTCLVKGFYPSDIAVEWESNGQPENNYKTTPP |
| | | | VLDSDGSFFLYSKLTVDKSRWQQGNVFSCSVMHEALHNHYTQK |
| | | | SLSLSPG |
| 1477 | ATX-P-3466 | HC IgG1-Fc aa | QVQLVQSGSELKKPGASVKVSCKASGYTFTSYTMNWVRQAPGQ |
| | | | GLEWVGWINTATGTPTYAQGFTGRYVFSLDTSVSTAYLQISGLK |
| | | | AEDTAVYFCARYSGSHSFDYWGQGTLVTVSSASTKGPSVFPLAPS |
| | | | SKSTSGGTAALGCLVKDYFPEPVTVSWNSGALTSGVHTFPAVLQ |
| | | | SSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDV |
| | | | SHEDPEVKFNWYVDGVEVHNAKTKPREEQYASTYRVVSVLTVL |
| | | | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPG |
| 1478 | ATX-P-3467 | HC IgG1-Fc aa | QVQLQESGPGLVKPSGTLSLTCAVSGGSISSDYWWSWVRQSPGK |
| | | | GLEWIGKIYHSGNTNHNPSLKSRVTISIDKSRNQFSLNLNSVTAAD |
| | | | TAVYYCAREGLELPHMDVWGKGTTVTVSSASTKGPSVFPLAPSS |
| | | | KSTSGGTAALGCLVKDYFPEPVTVSWNSGALTSGVHTFPAVLQS |
| | | | SGLYSLSSVVTVPSSSLGTQTYICNVNHKPSNTKVDKRVEPKSCD |
| | | | KTHTCPPCPAPELLGGPSVFLFPPKPKDTLMISRTPEVTCVVVDVS |
| | | | HEDPEVKFNWYVDGVEVHNAKTKPREEQYASTYRVVSVLTVLH |
| | | | QDWLNGKEYKCKVSNKALPAPIEKTISKAKGQPREPQVYTLPPSR |
| | | | EEMTKNQVSLTCLVKGFYPSDIAVEWESNGQPENNYKTTPPVLD |
| | | | SDGSFFLYSKLTVDKSRWQQGNVFSCSVMHEALHNHYTQKSLSL |
| | | | SPG |
| 1479 | ATX-P-3468 | HC IgG1-Fc aa | QVQLVESGGGLVQPGRSLRLSCAASGFTFSSYAMHWVRQAPGK |
| | | | GLEWVTVISYDGSNKYYADSVKGRFTISRDNSKNTLYLQMNSLR |
| | | | AEDTAVYYCAKRGNWNYGPFHYWGQGTLVTVSSASTKGPSVFP |
| | | | LAPSSKSTSGGTAALGCLVKDYFPEPVTVSWNSGALTSGVHTFPA |
| | | | VLQSSGLYSLSSVVTVPSSSLGTQTYICNVNHKPSNTKVDKRVEP |
| | | | KSCDKTHTCPPCPAPELLGGPSVFLFPPKPKDTLMISRTPEVTCVV |
| | | | VDVSHEDPEVKFNWYVDGVEVHNAKTKPREEQYASTYRVVSVL |
| | | | TVLHQDWLNGKEYKCKVSNKALPAPIEKTISKAKGQPREPQVYT |
| | | | LPPSREEMTKNQVSLTCLVKGFYPSDIAVEWESNGQPENNYKTTP |
| | | | PVLDSDGSFFLYSKLTVDKSRWQQGNVFSCSVMHEALHNHYTQ |
| | | | KSLSLSPG |
| 1480 | ATX-P-3469 | HC IgG1-Fc aa | QVQLVESGGGVVQPGRSLRLSCAASGFTFSAYAIHWVRQAPGKG |
| | | | LEWVAVISYDGSNKKYADSVKGRFTISRDNSKNTLDLEMNSLRA |
| | | | EDTAVYYCAKGLPFDYWGQGTLVTVSSASTKGPSVFPLAPSSKST |
| | | | SGGTAALGCLVKDYFPEPVTVSWNSGALTSGVHTFPAVLQSSGL |
| | | | YSLSSVVTVPSSSLGTQTYICNVNHKPSNTKVDKRVEPKSCDKTH |
| | | | TCPPCPAPELLGGPSVFLFPPKPKDTLMISRTPEVTCVVVDVSHED |
| | | | PEVKFNWYVDGVEVHNAKTKPREEQYASTYRVVSVLTVLHQD |
| | | | WLNGKEYKCKVSNKALPAPIEKTISKAKGQPREPQVYTLPPSREE |
| | | | MTKNQVSLTCLVKGFYPSDIAVEWESNGQPENNYKTTPPVLDSD |
| | | | GSFFLYSKLTVDKSRWQQGNVFSCSVMHEALHNHYTQKSLSLSP |
| | | | G |
| 1481 | ATX-P-3470 | HC IgG1-Fc aa | QVQLVQSGSDLKKPGASVKVSCKASGYTFTSYTMNWVRQAPGQ |
| | | | GLEWMGWINTNTGTPTYAQGFTGRFVFSLDTSVSTAYLQISSLKA |
| | | | EDTAVYYCTRYTGNYSFDYWGQGTLVTVSSASTKGPSVFPLAPS |
| | | | SKSTSGGTAALGCLVKDYFPEPVTVSWNSGALTSGVHTFPAVLQ |
| | | | SSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDV |
| | | | SHEDPEVKFNWYVDGVEVHNAKTKPREEQYASTYRVVSVLTVL |
| | | | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPG |
| 1482 | ATX-P-3471 | HC IgG1-Fc aa | EVQLVESGGVLVQPGGSLRLSCAASGFTFRSYAMSWVRQAPGKG |
| | | | LEWVSAISGSGDSTYFADSVKGRFTISRDNSKNTLSLQMNSLRAE |
| | | | DTAVYYCASILGYCSGTSCYGLEDNWGQGTLVTVSSASTKGPSV |
| | | | FPLAPSSKSTSGGTAALGCLVKDYFPEPVTVSWNSGALTSGVHTF |
| | | | PAVLQSSGLYSLSSVVTVPSSSLGTQTYICNVNHKPSNTKVDKRV |
| | | | EPKSCDKTHTCPPCPAPELLGGPSVFLFPPKPKDTLMISRTPEVTC |
| | | | VVVDVSHEDPEVKFNWYVDGVEVHNAKTKPREEQYASTYRVVS |
| | | | VLTVLHQDWLNGKEYKCKVSNKALPAPIEKTISKAKGQPREPQV |
| | | | YTLPPSREEMTKNQVSLTCLVKGFYPSDIAVEWESNGQPENNYK |
| | | | TTPPVLDSDGSFFLYSKLTVDKSRWQQGNVFSCSVMHEALHNHY |
| | | | TQKSLSLSPG |
| 1483 | ATX-P-3472 | HC IgG1-Fc aa | QVQLQESGPGLVKPSGTLSLTCAVSGGSISNDNWWSWVRQSPGK |
| | | | GLEWIGKIYHSEFTNYNPSLRSRVTISVDKSKNQFSLELTSVTAAD |
| | | | TAVYYCAREYQNYMDVWGKGTTVTVSSASTKGPSVFPLAPSSKS |
| | | | TSGGTAALGCLVKDYFPEPVTVSWNSGALTSGVHTFPAVLQSSG |
| | | | LYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVYTLPPSREE |
| | | | MTKNQVSLTCLVKGFYPSDIAVEWESNGQPENNYKTTPPVLDSD |
| | | | GSFFLYSKLTVDKSRWQQGNVFSCSVMHEALHNHYTQKSLSLSP |
| | | | G |
| 1484 | ATX-P-3361 | HC IgG1-Fc aa | QVQLQESGPGLVKPSGTLSLTCVVSGDSISSNNWWSWVRQPPGK |
| | | | GLEWIGQIYHSGITNYNPSLKSRVTISVDKSKNQFSLELTSVTAAD |
| | | | AAVYYCARAQWELRAGWFDPWGQGTLVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1485 | ATX-P-3366 | HC IgG1-Fc aa | EVQLVESGGGLVKPGGTLTLSCAASGFTFSNAWMSWVRQAPGK |
| | | | GLEWVGRIKSKTDGGTTDYAAPVKGRFTISRDDSKNTLYLQMNS |
| | | | LKTEDTAVYYCTPRYNSDFDYWGQGTLVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1486 | ATX-P-3376 | HC IgG1-Fc aa | QVQLQESGPGLVKPSETLSLTCTVSGGSISSYYWSWIRQPPGKGL |
| | | | EWIGYIYYSGFTKYNPSLKSRVTISVDTSKNQFSLKLSSVTAADTA |
| | | | VYYCATHPRGYNYGPFDYWGQGTLVTVSSASTKGPSVFPLAPSS |
| | | | KSTSGGTAALGCLVKDYFPEPVTVSWNSGALTSGVHTFPAVLQS |
| | | | SGLYSLSSVVTVPSSSLGTQTYICNVNHKPSNTKVDKRVEPKSCD |
| | | | KTHTCPPCPAPELLGGPSVFLFPPKPKDTLMISRTPEVTCVVVDVS |
| | | | HEDPEVKFNWYVDGVEVHNAKTKPREEQYASTYRVVSVLTVLH |
| | | | QDWLNGKEYKCKVSNKALPAPIEKTISKAKGQPREPQVYTLPPSR |
| | | | EEMTKNQVSLTCLVKGFYPSDIAVEWESNGQPENNYKTTPPVLD |
| | | | SDGSFFLYSKLTVDKSRWQQGNVFSCSVMHEALHNHYTQKSLSL |
| | | | SPG |
| 1487 | ATX-P-3377 | HC IgG1-Fc aa | QVQLQESGPGLVKPSETLSLTCTVSGGSVNSGRYYWSWIRQPPG |
| | | | KGLEWIGYIYSSGYTNYNPSLKSRVTMLLDTSKNQFSLKLNSVTA |
| | | | ADTAVYYCARSRGYRYGPFDYWGQGTLVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1488 | ATX-P-3418 | HC IgG1-Fc aa | QVQLVQSGSELKKPGASVKVSCEASGYTFTSYAMNWVRQAPGQ |
| | | | GLEWMGWINTNTGTPTYAQGFTGRFVFSLDTSVSTAYLQISSLKA |
| | | | EDTAVYYCASHSTSFDYWGQGTLVTVSSASTKGPSVFPLAPSSKS |
| | | | TSGGTAALGCLVKDYFPEPVTVSWNSGALTSGVHTFPAVLQSSG |
| | | | LYSLSSVVTVPSSSLGTQTYICNVNHKPSNTKVDKRVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYASTYRVVSVLTVLHQD |
| | | | WLNGKEYKCKVSNKALPAPIEKTISKAKGQPREPQVYTLPPSREE |
| | | | MTKNQVSLTCLVKGFYPSDIAVEWESNGQPENNYKTTPPVLDSD |
| | | | GSFFLYSKLTVDKSRWQQGNVFSCSVMHEALHNHYTQKSLSLSP |
| | | | G |
| 1489 | ATX-P-3420 | HC IgG1-Fc aa | QVQLQQWGAGLVKPSETLSLTCAVHGGSFSDYYLTWIRQPPGKG |
| | | | LEWIGEINHSGSTNYNPSLKSRVTLSIDTSKNQFSLKLSSVTAADT |
| | | | AVYYCARGSEDYYYYMDVWGKGTTVTVSSASTKGPSVFPLAPS |
| | | | SKSTSGGTAALGCLVKDYFPEPVTVSWNSGALTSGVHTFPAVLQ |
| | | | SSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDV |
| | | | SHEDPEVKFNWYVDGVEVHNAKTKPREEQYASTYRVVSVLTVL |
| | | | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPG |
| 1490 | ATX-P-3426 | HC IgG1-Fc aa | EVQLVQSGAEVKKPGESLKISCKGSGYIFTSYWIGWVRQMPGKG |
| | | | LEWMGIIYPGDSDTRYSPSFQGQVTISADKSIITAYLQWSSLKASD |
| | | | TAMYYCARHDYFDIGGYFDYWGQGTLVTVSSASTKGPSVFPLAP |
| | | | SSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1491 | ATX-P-3432 | HC IgG1-Fc aa | QVQLVQSGAEVKKPGASVKVSCKADGYTFTDYYMHWVRQAPG |
| | | | QGHEWMGWINPNSGVTYYAQKFQGRVTMTRDTSISTAYMELNR |
| | | | LRSDDTAVYYCARDRTFYFDYWGQGTLVTVSSASTKGPSVFPLA |
| | | | PSSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1492 | ATX-P-3433 | HC IgG1-Fc aa | QVQLVQSGAEVKKPGASVKVSCKASGYTFTDYYMHWVRQAPG |
| | | | QGLEWMGWINPNNGGTDYAQKFQGRVTMTRDTSISTAYMELHR |
| | | | LRSDDTAVYFCARGVYDVVVPTAIVNWEDPWGQGTLVTVSSAS |
| | | | TKGPSVFPLAPSSKSTSGGTAALGCLVKDYFPEPVTVSWNSGALT |
| | | | SGVHTFPAVLQSSGLYSLSSVVTVPSSSLGTQTYICNVNHKPSNTK |
| | | | VDKRVEPKSCDKTHTCPPCPAPELLGGPSVFLFPPKPKDTLMISRT |
| | | | PEVTCVVVDVSHEDPEVKFNWYVDGVEVHNAKTKPREEQYAST |
| | | | YRVVSVLTVLHQDWLNGKEYKCKVSNKALPAPIEKTISKAKGQP |
| | | | REPQVYTLPPSREEMTKNQVSLTCLVKGFYPSDIAVEWESNGQPE |
| | | | NNYKTTPPVLDSDGSFFLYSKLTVDKSRWQQGNVFSCSVMHEAL |
| | | | HNHYTQKSLSLSPG |
| 1493 | ATX-P-3438 | HC IgG1-Fc aa | QVQLQESGPGLVKPSGTLSLTCAVSGDSISNDYWWNWVRQPPGK |
| | | | GLEWIGEIYHSGHTNYNPSLKSRVTISVDKSKNQLSLKLTSVNAA |
| | | | DTAVYYCARGGTEITGYMDVWGKGTTVTVSSASTKGPSVFPLAP |
| | | | SSKSTSGGTAALGCLVKDYFPEPVTVSWNSGALTSGVHTFPAVL |
| | | | QSSGLYSLSSVVTVPSSSLGTQTYICNVNHKPSNTKVDKRVEPKS |
| | | | CDKTHTCPPCPAPELLGGPSVFLFPPKPKDTLMISRTPEVTCVVVD |
| | | | VSHEDPEVKFNWYVDGVEVHNAKTKPREEQYASTYRVVSVLTV |
| | | | LHQDWLNGKEYKCKVSNKALPAPIEKTISKAKGQPREPQVYTLPP |
| | | | SREEMTKNQVSLTCLVKGFYPSDIAVEWESNGQPENNYKTTPPV |
| | | | LDSDGSFFLYSKLTVDKSRWQQGNVFSCSVMHEALHNHYTQKSL |
| | | | SLSPG |
| 1494 | ATX-P-3441 | HC IgG1-Fc aa | QVQLQESGPGLVKPSGTLSLTCAVSGASIRSGHWWSWVRQPPGK |
| | | | GLEWIGEIYHSGNTNYNPSLKSRVTLSVDKSKNQFSLKLSSVTAA |
| | | | DTAVYYCARGHYGDYVVYFDYWGQGTLVTVSSASTKGPSVFPL |
| | | | APSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPK |
| | | | SCDKTHTCPPCPAPELLGGPSVFLFPPKPKDTLMISRTPEVTCVVV |
| | | | DVSHEDPEVKFNWYVDGVEVHNAKTKPREEQYASTYRVVSVLT |
| | | | VLHQDWLNGKEYKCKVSNKALPAPIEKTISKAKGQPREPQVYTL |
| | | | PPSREEMTKNQVSLTCLVKGFYPSDIAVEWESNGQPENNYKTTPP |
| | | | VLDSDGSFFLYSKLTVDKSRWQQGNVFSCSVMHEALHNHYTQK |
| | | | SLSLSPG |
| 1495 | ATX-P-3446 | HC IgG1-Fc aa | QVQLQESGPGLVKPSETLSLTCTVSGGSISSYYWSWIRQPPGKGL |
| | | | EWIGYVYYSGYTKYNPSLKSRVTISIDASKNQFSLKLTSVTAADT |
| | | | AVYYCATVESKWNYGPFAIWGQGTMVTVSSASTKGPSVFPLAPS |
| | | | SKSTSGGTAALGCLVKDYFPEPVTVSWNSGALTSGVHTFPAVLQ |
| | | | SSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDV |
| | | | SHEDPEVKFNWYVDGVEVHNAKTKPREEQYASTYRVVSVLTVL |
| | | | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPG |
| 1496 | ATX-P-3452 | HC IgG1-Fc aa | QVQLVQSGAEVKKPGASVKVSCKASGYTFTGYYMNWVRQAPG |
| | | | QGLEWMGWINPNSGATNYAQKFQGRVTMTRDTSISTDYMELSR |
| | | | LRSDDTAVYYCARDGAKWNHEGYYFDYWGQGTLVTVSSASTK |
| | | | GPSVFPLAPSSKSTSGGTAALGCLVKDYFPEPVTVSWNSGALTSG |
| | | | VHTFPAVLQSSGLYSLSSVVTVPSSSLGTQTYICNVNHKPSNTKV |
| | | | DKRVEPKSCDKTHTCPPCPAPELLGGPSVFLFPPKPKDTLMISRTP |
| | | | EVTCVVVDVSHEDPEVKFNWYVDGVEVHNAKTKPREEQYASTY |
| | | | RVVSVLTVLHQDWLNGKEYKCKVSNKALPAPIEKTISKAKGQPR |
| | | | EPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALH |
| | | | NHYTQKSLSLSPG |

TABLE 10
Anti-C3 antibodies (LC Kappa sequences)

| SEQ ID | Antibody | | |
|---|---|---|---|
| NO: | Name: | Descriptor: | Sequence: |
| 1497 | ATX-P-3360 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1498 | ATX-P-3362 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1499 | ATX-P-3363 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1500 | ATX-P-3365 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1501 | ATX-P-3367 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1502 | ATX-P-3369 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1503 | ATX-P-3370 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1504 | ATX-P-3371 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1505 | ATX-P-3373 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1506 | ATX-P-3375 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1507 | ATX-P-3378 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1508 | ATX-P-3382 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1509 | ATX-P-3383 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1510 | ATX-P-3384 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1511 | ATX-P-3386 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1512 | ATX-P-3387 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1513 | ATX-P-3389 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1514 | ATX-P-3391 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1515 | ATX-P-3392 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1516 | ATX-P-3395 | LC Kappa nt | GACATCCAGATGACCCAGTCTCCTTCCACCCTGTCTGCAT |
| | | | CTGTAGGAGACAGCGTCACCATCACTTGCCGGGCCAGTCA |
| | | | GAATATTATTACCTGGTTGGCTTGGTATCAGCAGAAACCA |
| | | | GGGAAAGCCCCTAACCTCCTGATCTATAAGGCGTCTAGTT |
| | | | TAGAAAGTGGGGTCCCATCAAGGTTCAGCGGCAGTGGATC |
| | | | TGGGACAGAATTCACTCTCACCATCAGCAGCCTGCAGCCT |
| | | | GATGATTTTGCAACTTATTACTGCCAACAGTATAGTAGTT |
| | | | ATTCGTACACTTTTGGCCAGGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1517 | ATX-P-3396 | LC Kappa nt | GACATCCAGATGACCCAGTCTCCTTCCACCCTGTCTGCAT |
| | | | CTGTAGGAGACAGCGTCACCATCACTTGCCGGGCCAGTCA |
| | | | GAATATTATTACCTGGTTGGCTTGGTATCAGCAGAAACCA |
| | | | GGGAAAGCCCCTAACCTCCTGATCTATAAGGCGTCTAGTT |
| | | | TAGAAAGTGGGGTCCCATCAAGGTTCAGCGGCAGTGGATC |
| | | | TGGGACAGAATTCACTCTCACCATCAGCAGCCTGCAGCCT |
| | | | GATGATTTTGCAACTTATTACTGCCAACAGTATAGTAGTT |
| | | | ATTCGTACACTTTTGGCCAGGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1518 | ATX-P-3397 | LC Kappa nt | GACATCCAGATGACCCAGTCTCCTTCCACCCTGTCTGCAT |
| | | | CTGTAGGAGACAGCGTCACCATCACTTGCCGGGCCAGTCA |
| | | | GAATATTATTACCTGGTTGGCTTGGTATCAGCAGAAACCA |
| | | | GGGAAAGCCCCTAACCTCCTGATCTATAAGGCGTCTAGTT |
| | | | TAGAAAGTGGGGTCCCATCAAGGTTCAGCGGCAGTGGATC |
| | | | TGGGACAGAATTCACTCTCACCATCAGCAGCCTGCAGCCT |
| | | | GATGATTTTGCAACTTATTACTGCCAACAGTATAGTAGTT |
| | | | ATTCGTACACTTTTGGCCAGGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1519 | ATX-P-3405 | LC Kappa nt | GACATCCAGATGACCCAGTCTCCTTCCACCCTGTCTGCAT |
| | | | CTGTAGGAGACAGCGTCACCATCACTTGCCGGGCCAGTCA |
| | | | GAATATTATTACCTGGTTGGCTTGGTATCAGCAGAAACCA |
| | | | GGGAAAGCCCCTAACCTCCTGATCTATAAGGCGTCTAGTT |
| | | | TAGAAAGTGGGGTCCCATCAAGGTTCAGCGGCAGTGGATC |
| | | | TGGGACAGAATTCACTCTCACCATCAGCAGCCTGCAGCCT |
| | | | GATGATTTTGCAACTTATTACTGCCAACAGTATAGTAGTT |
| | | | ATTCGTACACTTTTGGCCAGGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1520 | ATX-P-3406 | LC Kappa nt | GACATCCAGATGACCCAGTCTCCTTCCACCCTGTCTGCAT |
| | | | CTGTAGGAGACAGCGTCACCATCACTTGCCGGGCCAGTCA |
| | | | GAATATTATTACCTGGTTGGCTTGGTATCAGCAGAAACCA |
| | | | GGGAAAGCCCCTAACCTCCTGATCTATAAGGCGTCTAGTT |
| | | | TAGAAAGTGGGGTCCCATCAAGGTTCAGCGGCAGTGGATC |
| | | | TGGGACAGAATTCACTCTCACCATCAGCAGCCTGCAGCCT |
| | | | GATGATTTTGCAACTTATTACTGCCAACAGTATAGTAGTT |
| | | | ATTCGTACACTTTTGGCCAGGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1521 | ATX-P-3407 | LC Kappa nt | GACATCCAGATGACCCAGTCTCCTTCCACCCTGTCTGCAT |
| | | | CTGTAGGAGACAGCGTCACCATCACTTGCCGGGCCAGTCA |
| | | | GAATATTATTACCTGGTTGGCTTGGTATCAGCAGAAACCA |
| | | | GGGAAAGCCCCTAACCTCCTGATCTATAAGGCGTCTAGTT |
| | | | TAGAAAGTGGGGTCCCATCAAGGTTCAGCGGCAGTGGATC |
| | | | TGGGACAGAATTCACTCTCACCATCAGCAGCCTGCAGCCT |
| | | | GATGATTTTGCAACTTATTACTGCCAACAGTATAGTAGTT |
| | | | ATTCGTACACTTTTGGCCAGGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1522 | ATX-P-3408 | LC Kappa nt | GACATCCAGATGACCCAGTCTCCTTCCACCCTGTCTGCAT |
| | | | CTGTAGGAGACAGCGTCACCATCACTTGCCGGGCCAGTCA |
| | | | GAATATTATTACCTGGTTGGCTTGGTATCAGCAGAAACCA |
| | | | GGGAAAGCCCCTAACCTCCTGATCTATAAGGCGTCTAGTT |
| | | | TAGAAAGTGGGGTCCCATCAAGGTTCAGCGGCAGTGGATC |
| | | | TGGGACAGAATTCACTCTCACCATCAGCAGCCTGCAGCCT |
| | | | GATGATTTTGCAACTTATTACTGCCAACAGTATAGTAGTT |
| | | | ATTCGTACACTTTTGGCCAGGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1523 | ATX-P-3410 | LC Kappa nt | GACATCCAGATGACCCAGTCTCCTTCCACCCTGTCTGCAT |
| | | | CTGTAGGAGACAGCGTCACCATCACTTGCCGGGCCAGTCA |
| | | | GAATATTATTACCTGGTTGGCTTGGTATCAGCAGAAACCA |
| | | | GGGAAAGCCCCTAACCTCCTGATCTATAAGGCGTCTAGTT |
| | | | TAGAAAGTGGGGTCCCATCAAGGTTCAGCGGCAGTGGATC |
| | | | TGGGACAGAATTCACTCTCACCATCAGCAGCCTGCAGCCT |
| | | | GATGATTTTGCAACTTATTACTGCCAACAGTATAGTAGTT |
| | | | ATTCGTACACTTTTGGCCAGGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GTGGTAGCTGCCCCTTCAGTTTTTATCTTTCCGCCGTCTG |
| | | | ACGAGCAGTTAAAATCCGGGACCGCTTCTGTAGTTTGCCT |
| | | | GCTGAATAATTTTTATCCGCGTGAGGCTAAAGTACAATGG |
| | | | AAAGTCGACAATGCTTTGCAGTCGGGAAATTCACAGGAAA |
| | | | GTGTTACGGAGCAGGATTCTAAAGATTCCACATATTCACT |
| | | | CAGCTCCACCCTTACACTGAGCAAAGCCGACTATGAAAAA |
| | | | CATAAAGTTTACGCATGTGAGGTGACGCACCAAGGATTAT |
| | | | CCAGTCCGGTCACAAAATCGTTTAACCGCGGTGAGTGT |
| 1528 | ATX-P-3421 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GACTGTTTTGTACAGGTCCAACAATAAGAACTACTTAGCT |
| | | | TGGTATCAGCAGAAACCAGGACAGCCTCCTAAGCTGCTCA |
| | | | TTTACTGGGCATCTACCCGGGAATCCGGGGTCCCTGACCG |
| | | | ATTCAGTGGCAGCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTGGACGTTCGGCCAAGG |
| | | | GACCAAGGTGGAAATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1529 | ATX-P-3428 | LC Kappa nt | GCCATCCAGATGACCCAGTCTCCATCCTCCCTGTCTGCTT |
| | | | CTGTAGGAGACAGAGTCACCATCACTTGCCGGGCAAGTCA |
| | | | GGGCATTAGAAATGATTTAGGCTGGTATCAGCAGAATCCC |
| | | | GGGAAAGCCCCTAAGCTCCTGATTTATGCTGCATCCAATT |
| | | | TACAAAGTGGGGTCCCATCAAGGTTCAGCGGCAGTGGATC |
| | | | TGGCACAGATTTCACTCTCACCATCAGCAGCCTGCAGCCT |
| | | | GAAGATTTTGTTACTTATTACTGTCTACAAGATTACAATT |
| | | | GGCCGTGGACGTTCGGCCAAGGGACACGACTGGAGATTAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1530 | ATX-P-3430 | LC Kappa nt | GACATCCAGATGACCCAGTCTCCATCCTCCCTGTCTGCAT |
| | | | CTGTAGGAGACAGAGTCACCATCACTTGCCGGGCAAGTCA |
| | | | GAGCATTAGCAACTATTTAAATTGGTATCAACAGAAACCA |
| | | | GAGAAAGCCCCTAAGTTCCTGATCTATGCTGCATCCAGTT |
| | | | TGCAAAGTGGGGTCCCATCAAGGTTCAGTGGCAGTGGATC |
| | | | TGGGACAGATTTCACTCTCACCATCAGCAGTCTGCAACCT |
| | | | GAAGATTTTGCAACTTACTACTGTCAACAGAGTTACAGTT |
| | | | CCCCGCTCACTTTCGGCGGAGGGACCAAGGTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1531 | ATX-P-3431 | LC Kappa nt | GAAATTGTGTTGACTCAGTCTCCAGACTTTCAGTCTGTGA |
| | | | CTCCAAAGGAGAAAGTCACCATCACCTGCCGGGCCAGTCA |
| | | | GAGCATTGGTAGTAACTTACACTGGTTCCAGCAGAAACCA |
| | | | GATCAGTCTCCAAAACTCCTCATCAAGTATGCTTCCCAGT |
| | | | CCTTCTCAGGGGTCCCCTCGAGGTTCAGTGGCAGTGGATC |
| | | | TGGGACAGATTTCACCCTCACCATCAATAGCCTGGAAGCT |
| | | | GAAGATGCTGCAATGTATTACTGTCATCAGAGTAGTAGTT |
| | | | TACCTCGGACGTTCGGCCAAGGGACCAAGGTGGAAATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1532 | ATX-P-3435 | LC Kappa nt | GCCATCCAGATGACCCAGTCTCCATCCTCCCTGTCTGCAT |
| | | | CTGTAGGAGACAGAGTCACCATCACTTGCCGGGCAAGTCA |
| | | | GGGCATTAGAAATGATTTAGGCTGGTATCAGCAGAAACCA |
| | | | GGGAAAGCCCCTAAGCTCCTGATCTATGCTGCATCCAGTT |
| | | | TACAAAGTGGGGTCCCATCAAGGTTCAGCGGCAGTGGATC |
| | | | TGGCACAGATTTCACTCTCACCATCAGCAGCCTGCAGCCT |
| | | | GAAGATTTTGCATCTTATTACTGTCTACAAGATTACAATT |
| | | | GGCCGTACACTTTTGGCCAGGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1533 | ATX-P-3436 | LC Kappa nt | GAAATTGTGTTGACGCAGTCTCCAGGCACCCTGTCTTTGT |
| | | | CTCCAGGGGAAAGAGCCACCCTCTCCTGCAGGGCCAGTCA |
| | | | GAGTGTTAGAAGCAGCTACTTTGCCTGGTACCAGCAGAAA |
| | | | CCTGGCCAGGCTCCCAGGCTCCTCATCTATGGTGCATCCA |
| | | | GCAGGGCCACTGGCATCCCAGACAGGTTCAGTGGCAGTGG |
| | | | GTCTGGGACAGACTTCACTCTCACCATCAGCAGACTGGAG |
| | | | CCTGAAGATTTTGCAGTGTATTACTGTCAGCAGTATGGTA |
| | | | GCTCACCTCGGACGTTCGGCCAAGGGACCAAGGTGGAAAT |
| | | | CAAACGTACGGTAGCTGCCCCTTCAGTTTTTATCTTTCCG |
| | | | CCGTCTGACGAGCAGTTAAAATCCGGGACCGCTTCTGTAG |
| | | | TTTGCCTGCTGAATAATTTTTATCCGCGTGAGGCTAAAGT |
| | | | ACAATGGAAAGTCGACAATGCTTTGCAGTCGGGAAATTCA |
| | | | CAGGAAAGTGTTACGGAGCAGGATTCTAAAGATTCCACAT |
| | | | ATTCACTCAGCTCCACCCTTACACTGAGCAAAGCCGACTA |
| | | | TGAAAAACATAAAGTTTACGCATGTGAGGTGACGCACCAA |
| | | | GGATTATCCAGTCCGGTCACAAAATCGTTTAACCGCGGTG |
| | | | AGTGT |
| 1534 | ATX-P-3437 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTACCCGGGAATCCGGGGTCCCTGACCG |
| | | | ATTCAGTGGCAGCGGGTCTGGGACAGATTTCACTCTCACC |
| | | | ATCAGCAGCCTGCAGGCTGAAGATGTGGCAGTTTATTACT |
| | | | GTCAGCAATATTTTGGTACTCCTCGGACGTTCGGCCAAGG |
| | | | GACCAAGGTGGAAATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1535 | ATX-P-3440 | LC Kappa nt | GAAATTGTGTTGACACAGTCTCCAGCCACCCTGTCTTTGT |
| | | | CTCCAGGGGAAAGAGCCACCCTCTCCTGCAGGGCCAGTCA |
| | | | GAGTGTTAGCAGCTACTTAGCCTGGTACCAACAGAAACCT |
| | | | GGCCAGGCTCCCAGGCTCCTCATCTATGATGCATCCAACA |
| | | | GGGCCACTGGCATCCCAGCCAGGTTCAGTGGCAGTGGGTC |
| | | | TGGGACAGACTTCACTCTCACCATCAGCAGCCTGCAGTCT |
| | | | GAAGATTTTGCAGTTTATTACTGTCAGCAGCGTAGCAATT |
| | | | GGCCGCTCACTTTCGGCGGAGGGACCAAGGTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1536 | ATX-P-3442 | LC Kappa nt | GACATCCAGATGACCCAGTCTCCATCCTCCCTGTCTGCAT |
| | | | CTGTAGGAGACAGAGTCACCATCACTTGCCGGGCAAGTCA |
| | | | GACCATTAGGAGCTATTTAAATTGGTATCAGCAGAAACCA |
| | | | GGGAAAGCCCCTAAGCTCCTGATCTATGCTGCATCCAGTT |
| | | | TGCAAAGTGGGGTCCCATCAAGGTTCAGTGGCAGTGGATC |
| | | | TGGGACAAATTTCACTCTCACCATCAGCAGTCTGCAACCT |
| | | | GAAGATTTTGCAACTTACTCCTGTCAACAGAGTTACACTA |
| | | | CCCCGCTCACTTTCGGCGGAGGGACCAAGGTGGAGATCAA |
| | | | ACGTACGGTAGCTGCCCCTTCAGTTITTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1537 | ATX-P-3444 | LC Kappa nt | GAAATTGTGTTGACGCAGTCTCCAGGCACCCTGTCTTTGT |
| | | | CTCCAGGGGAAAGAGCCACCCTCTCCTGCAGGGCCAGTCA |
| | | | GAGTATTAGCAGCAGCTACTTAGCCTGGTACCAGCGGGAA |
| | | | CCTGGCCAGGCTCCCAGGCTCCTCATCTATGGTGCATCCA |
| | | | GCAGGGCCACTGGCATCCCAGACAGGTTCAGTGGCAGTGG |
| | | | GTCTGGGACAGACTTCACTCTCACCATCAGCAGACTGGAG |
| | | | CCTGAAGATTTTGTAGTGTATTACTGTCAACAATATGGTA |
| | | | GCTCACCGATCACCTTCGGCCAAGGGACACGACTGGAGAT |
| | | | TAAACGTACGGTAGCTGCCCCTTCAGTTTTTATCTTTCCG |
| | | | CCGTCTGACGAGCAGTTAAAATCCGGGACCGCTTCTGTAG |
| | | | TTTGCCTGCTGAATAATTTTTATCCGCGTGAGGCTAAAGT |
| | | | ACAATGGAAAGTCGACAATGCTTTGCAGTCGGGAAATTCA |
| | | | CAGGAAAGTGTTACGGAGCAGGATTCTAAAGATTCCACAT |
| | | | ATTCACTCAGCTCCACCCTTACACTGAGCAAAGCCGACTA |
| | | | TGAAAAACATAAAGTTTACGCATGTGAGGTGACGCACCAA |
| | | | GGATTATCCAGTCCGGTCACAAAATCGTTTAACCGCGGTG |
| | | | AGTGT |
| 1538 | ATX-P-3447 | LC Kappa nt | GAAATTGTGTTGACGCAGTCTCCAGGCACCCTGTCTTTGT |
| | | | CTCCAGGGGAAAGAGCCACCCTCTCCTGCAGGGCCAGTCA |
| | | | GAGTGTTCGCAGCAGCTACTTAGCCTGGTACCAGCAGAAA |
| | | | CCTGGCCAGGCTCCCAGGCTCCTCATCTATGGTGCATCCA |
| | | | GCAGGGCCACTGGCATCCCAGACAGGTTCAGTGGCAGTGG |
| | | | GTCTGGGACAGACTTCACTCTCACCTTCAGCAGACTGGAG |
| | | | CCTGAAGATTTTGCAGTGTATTACTGTCAGCAGTATGGTA |
| | | | GCTCACCGTACACTTTTGGCCAGGGGACCAAGCTGGAGAT |
| | | | CAAACGTACGGTAGCTGCCCCTTCAGTTTTTATCTTTCCG |
| | | | CCGTCTGACGAGCAGTTAAAATCCGGGACCGCTTCTGTAG |
| | | | TTTGCCTGCTGAATAATTTTTATCCGCGTGAGGCTAAAGT |
| | | | ACAATGGAAAGTCGACAATGCTTTGCAGTCGGGAAATTCA |
| | | | CAGGAAAGTGTTACGGAGCAGGATTCTAAAGATTCCACAT |
| | | | ATTCACTCAGCTCCACCCTTACACTGAGCAAAGCCGACTA |
| | | | TGAAAAACATAAAGTTTACGCATGTGAGGTGACGCACCAA |
| | | | GGATTATCCAGTCCGGTCACAAAATCGTTTAACCGCGGTG |
| | | | AGTGT |
| 1539 | ATX-P-3448 | LC Kappa nt | GACATCCAGATGACCCAGTCTCCATCCTCCCTGTCTGCAT |
| | | | CTGTAGGAGACAGAGTCACCATCACTTGCCAGGCGAGTCA |
| | | | GGACATTAGCAACTATTTAAATTGGTATCAGCAGAAACCA |
| | | | GGGAAAGCCCCTAAGCTCCTGATCTACGATGCATCCAATT |
| | | | TGGAAACAGGGGTCCCATCAAGGTTCAGTGGAAGTGGATC |
| | | | TGGGACAGATTTTACTTTCACCATCAGCAGCCTGCAGCCT |
| | | | GAAGATTTTGCAACTTACTATTGTCAACAGGCTAACAGTT |
| | | | TCCCGCTCACTTTCGGCGGAGGGACCAAGGTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1540 | ATX-P-3449 | LC Kappa nt | GAAATTGTGTTGACGCAGTCTCCAGGCACCCTGTCTTTGT |
| | | | CTCCAGGGGAAAGAGCCACCCTCTCCTGCAGGGCCAGTCA |
| | | | GAGTGTTAGCAGCAGCTACTTAGCCTGGTACCAGCAGAAA |
| | | | CCTGGCCAGGCTCCCAGGCTCCTCATCTATGGTGCATCCA |
| | | | GCAGGGCCACTGGCATCCCAGACAGGTTCAGTGGCAGTGG |
| | | | GTCTGGGACAGACTTCACTCTCACCATCAGCAGACTGGAG |
| | | | CCTGAAGATTTTGCAGTGTATTACTGTCAGCAGTATGGTA |
| | | | GCTCACCATTCACTTTCGGCCCTGGGACCAAAGTGGATAT |
| | | | CAAACGTACGGTAGCTGCCCCTTCAGTTTTTATCTTTCCG |
| | | | CCGTCTGACGAGCAGTTAAAATCCGGGACCGCTTCTGTAG |
| | | | TTTGCCTGCTGAATAATTTTTATCCGCGTGAGGCTAAAGT |
| | | | ACAATGGAAAGTCGACAATGCTTTGCAGTCGGGAAATTCA |
| | | | CAGGAAAGTGTTACGGAGCAGGATTCTAAAGATTCCACAT |
| | | | ATTCACTCAGCTCCACCCTTACACTGAGCAAAGCCGACTA |
| | | | TGAAAAACATAAAGTTTACGCATGTGAGGTGACGCACCAA |
| | | | GGATTATCCAGTCCGGTCACAAAATCGTTTAACCGCGGTG |
| | | | AGTGT |
| 1541 | ATX-P-3450 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTACCCGGGAATCCGGGGTCCCTGACCG |
| | | | ATTCAGTGGCAGCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTGGACGTTCGGCCAAGG |
| | | | GACACGACTGGAGATTAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1542 | ATX-P-3451 | LC Kappa nt | GACATCCAGATGACCCAGTCTCCATCCTCCCTGTCTGCAT |
| | | | CTGTAGGAGACAGAGTCACCATCACTTGCCGGGCGAGTCA |
| | | | GGACATTAGCAACTATTTAAATTGGTATCAGCAGAAACCA |
| | | | GGGAAAGCCCCTAAGCTCCTGATCTATGCTGCATCCAGTT |
| | | | TGGAAAGTGGGGTCCCATCAAGGTTCAGTGGCAGTGGATC |
| | | | TGGGACAGATTTCACTCTCACCATCAGCAGTCTGCAACCT |
| | | | GAAGATTTTGCAACTTACTACTGTCAACAGAGTTACAGTA |
| | | | CCCCTACTTTCGGCGGAGGGACCAAGGTGGAGATCAAACG |
| | | | TACGGTAGCTGCCCCTTCAGTTTTTATCTTTCCGCCGTCT |
| | | | GACGAGCAGTTAAAATCCGGGACCGCTTCTGTAGTTTGCC |
| | | | TGCTGAATAATTTTTATCCGCGTGAGGCTAAAGTACAATG |
| | | | GAAAGTCGACAATGCTTTGCAGTCGGGAAATTCACAGGAA |
| | | | AGTGTTACGGAGCAGGATTCTAAAGATTCCACATATTCAC |
| | | | TCAGCTCCACCCTTACACTGAGCAAAGCCGACTATGAAAA |
| | | | ACATAAAGTTTACGCATGTGAGGTGACGCACCAAGGATTA |
| | | | TCCAGTCCGGTCACAAAATCGTTTAACCGCGGTGAGTGT |
| 1543 | ATX-P-3453 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGGTCCAACAATAAGAACTACTTAGCT |
| | | | TGGTACCAGCAGAAACCAGGCCAGCCTCCTAAACTGCTCA |
| | | | TTTACTGGGCATCTACCCGGGAATCCGGGGTCCCTGACCG |
| | | | ATTCAGTGGCAGCGGGTCTGGGACAGATTTCACTCTCACC |
| | | | ATCAGCAGCCTGCAGGCTGAAGATGTGGCAGTTTATTACT |
| | | | GTCAGCAATATTATGGTACTCCTCGGACGTTCGGCCAAGG |
| | | | GACCAAGGTGGAAATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1544 | ATX-P-3454 | LC Kappa nt | GACATCCAGATGACCCAGTCTCCATCCTCCCTGTCTGCAT |
| | | | CTGTAGGAGACAGAGTCACCATCACTTGCCAGGCGAGTCA |
| | | | GGACATTAAGAACTGTTTAAATTGGTATCAGCAGAAACCA |
| | | | GGGAAAGCCCCTAAGCTCCTGATCTACGATGCATCCAATT |
| | | | TGGAAACAGGGGTCCCATCAAGGTTCAGTGGAAGTGGATC |
| | | | TGGGACAGATTTTACTTTCACCATCAGCAGCCTGCAGCCT |
| | | | GAAGATATTGCAACATATTACTGTCAACAGTTTGATAATC |
| | | | TCCCTCTCACTTTCGGCGGAGGGACCAAGGTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1545 | ATX-P-3455 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GACTATTTTATACAGGTCCAACATTAAGAACTACTTAGCT |
| | | | TGGTACCAGCAGAAACCAGGACAGCCTCCTAAACTGCTCA |
| | | | TTTCCTGGGCATCTACCCGGGAATCCGGGGTCCCTGACCG |
| | | | ATTCAGTGGCAGCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCGGACGTTCGGCCAGGGGAC |
| | | | CAAGCTGGAGATCAAACGTACGGTAGCTGCCCCTTCAGTT |
| | | | TTTATCTTTCCGCCGTCTGACGAGCAGTTAAAATCCGGGA |
| | | | CCGCTTCTGTAGTTTGCCTGCTGAATAATTTTTATCCGCG |
| | | | TGAGGCTAAAGTACAATGGAAAGTCGACAATGCTTTGCAG |
| | | | TCGGGAAATTCACAGGAAAGTGTTACGGAGCAGGATTCTA |
| | | | AAGATTCCACATATTCACTCAGCTCCACCCTTACACTGAG |
| | | | CAAAGCCGACTATGAAAAACATAAAGTTTACGCATGTGAG |
| | | | GTGACGCACCAAGGATTATCCAGTCCGGTCACAAAATCGT |
| | | | TTAACCGCGGTGAGTGT |
| 1546 | ATX-P-3456 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTACCCGGGAATCCGGGGTCCCTGACCG |
| | | | ATTCAGTGGCAGCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCTCGGACGTTCGGCCAAGG |
| | | | GACCAAGGTGGAAATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1547 | ATX-P-3458 | LC Kappa nt | GACATCCAGATGACCCAGTCTCCTTCCACCCTGTCTGCAT |
| | | | CTGTAGGAGACAGAGTCACTATCACTTGTCGGGCGAGTCA |
| | | | GGGTATTAGCAGCTGGTTAGCCTGGTATCAGCAGAAACCA |
| | | | GGGAAAGCCCCTAAACTCCTGATCTATGCTGCATCCAGTT |
| | | | TGCAGAGTGGGGTCCCATCAAGGTTCAGCGGCAGTGGATC |
| | | | TGGGACAGATTTCACTCTCACCATCAGCAGCCTGCAGCCT |
| | | | GAAGATTTTGCAACTTACTATTGTCAACAGGCTAACAGTT |
| | | | TCCCTCTCACTTTCGGCGGAGGGACCAAGGTGGAGATCAA |
| | | | ACGTACGGTAGCTGCCCCTTCAGTTITTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1548 | ATX-P-3459 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTACCCGGGAATCCGGGGTCCCTGACCG |
| | | | ATTCAGTGGCAGCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCTCCGACGTTCGGCCAAGG |
| | | | GACCAAGGTGGAAATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1549 | ATX-P-3462 | LC Kappa nt | GACATCCAGATGACCCAGTCTCCATCCTCCCTGTCTGCAT |
| | | | CTGTAGGAGACAGAGTCACCATCACTTGCCGGGCAAGTCA |
| | | | GAGCATTAGCAGCTATTTAAATTGGTATCAGCAGAAACCA |
| | | | GGGAAAGCCCCTAAGCTCCTGATCTATGCTGCATCCAGTT |
| | | | TGCAAAGTGGGGTCCCATCAAGGTTCAGTGGCAGTGGATC |
| | | | TGGGACAGATTTCACTCTCACCATCAGCAGTCTGCAACCT |
| | | | GAAGATTTTGCAACTTACTACTGTCAACAGAGTTACAGTA |
| | | | CCCCGTACACTTTTGGCCAGGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1550 | ATX-P-3463 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTACCCGGGAATCCGGGGTCCCTGACCG |
| | | | ATTCAGTGGCAGCGGGTCTGGGACAGATTTCACTCTCACC |
| | | | ATCAGCAGCCTGCAGGCTGAAGATGTGGCAGTTTATTACT |
| | | | GTCAGCAATTTTATGATACTCCTCGGACGTTCGGCCAAGG |
| | | | GACCAAGGTGGAAATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1551 | ATX-P-3466 | LC Kappa nt | GACATCCAGATGACCCAGTCTCCATCCTCCCTGTCTGCAT |
| | | | CTGTAGGAGACAGAGTCACCATCACTTGCCGGGCGAGTCA |
| | | | GGGCATTAGCAATTATTTAGCCTGGTATCAGCAGAAACCA |
| | | | GGGAAAGTTCCTAAGCTCCTGATCTATGTTGCATCCACTT |
| | | | TGCAATCAGGGGTCCCATCTCGGTTCAGTGGCAGTGGATC |
| | | | TGGGACAGATTTCACTCTCACCATCAGCAGCCTGCAGCCT |
| | | | GAAGATGTTGCAACTTATTACTGTCAAAATTATATCAGTG |
| | | | CCCCGCTCACTTTCGGCGGAGGGACCAAGGTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1552 | ATX-P-3467 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTACCCGGGAATCCGGGGTCCCTGACCG |
| | | | ATTCAGTGGCAGCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTGGACGTTCGGCCAAGG |
| | | | GACCAAGGTGGAAATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1553 | ATX-P-3468 | LC Kappa nt | GAAATTGTGTTGACGCAGTCTCCAGGCACCCTGTCTTTGT |
| | | | CTCCAGGGGAAAGAGCCACCCTCTCCTGCAGGGCCAGTCA |
| | | | GAGTGTTAGCAGCAGCTACTTAGCCTGGTACCAGCAGAAA |
| | | | CCTGGCCAGGCTCCCAGGCTCCTCATCTATGGTGCATCCA |
| | | | GCAGGGCCACTGGCATCCCAGACAGGTTCAGTGGCAGTGG |
| | | | GTCTGGGACAGACTTCACTCTCACCATCAGCAGACTGGAG |
| | | | CCTGAAGATTTTGCAGTGTATTACTGTCAGCAGTATGGTA |
| | | | GCTCACCGTACACTTTTGGCCAGGGGACCAAGCTGGAGAT |
| | | | CAAACGTACGGTAGCTGCCCCTTCAGTTTTTATCTTTCCG |
| | | | CCGTCTGACGAGCAGTTAAAATCCGGGACCGCTTCTGTAG |
| | | | TTTGCCTGCTGAATAATTTTTATCCGCGTGAGGCTAAAGT |
| | | | ACAATGGAAAGTCGACAATGCTTTGCAGTCGGGAAATTCA |
| | | | CAGGAAAGTGTTACGGAGCAGGATTCTAAAGATTCCACAT |
| | | | ATTCACTCAGCTCCACCCTTACACTGAGCAAAGCCGACTA |
| | | | TGAAAAACATAAAGTTTACGCATGTGAGGTGACGCACCAA |
| | | | GGATTATCCAGTCCGGTCACAAAATCGTTTAACCGCGGTG |
| | | | AGTGT |
| 1554 | ATX-P-3469 | LC Kappa nt | GACATCCAGATGACCCAGTCTCCATCCTCCCTGTCTGCAT |
| | | | CTGTAGGAGACAGAGTCAACATCACTTGCCGGGCGAGTCA |
| | | | GGGCATTAGCAATTATTTAGCCTGGTATCAGCAGAAACCA |
| | | | GGGAAAGTTCCTAAGCTCCTGATCTATGCTGCATCCACTT |
| | | | TGCAATCAGGGGTCCCATCTCGTTTCAGTGGCAGTGGATC |
| | | | TGGGACAGATTTCACTCTCACCATCAGCAGCCTGCAGCCT |
| | | | GAAGATGTTGCAAGTTATTTCTGTCAAAAATATATCAGTG |
| | | | CCCCGCTCACTTTCGGCGGAGGGACCAAGGTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1555 | ATX-P-3470 | LC Kappa nt | GACATCCAGATGACCCAGTCTCCATCCTCCCTGTCTGCAT |
| | | | CTGTAGGAGACAGAGTCACCATCACTTGCCGGGCGAGTCA |
| | | | GGGCATTAGCAATTATTTAGCCTGGTATCAGCAGAAACCA |
| | | | GGGAAAGTTCCTAAGCTCCTGATCTATGCTGCATCCACTT |
| | | | TGCAATCAGGGGTCCCATCTCGGTTCAGTGGCAGTGGATC |
| | | | TGGGACAGATTTCACTCTCACCATCAGCAGCCTGCAGCCT |
| | | | GAAGACGTTGCAAGTTATTACTGTCAAGATTATACCAGTG |
| | | | CCCCGCTCACTTTCGGCGGAGGGACCAAGGTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1556 | ATX-P-3471 | LC Kappa nt | GAAATTGTGTTGACGCAGTCTCCAGGCACCCTGTCTTTGT |
| | | | CTCCAGGGGAAAGAGCCACCCTCTCCTGCAGGGCCAGTCA |
| | | | GAGTGTTAGTGACAGCTACTTAGCCTGGTACCAGCAGGAA |
| | | | CCTGGCCAGGCGCCCAGGCTCCTCATCTATGGTGCATCCA |
| | | | GCAGGGCCACTGGCATCCCAGACAGGTTCAGTGGCAGTGG |
| | | | GTCTGGGACAGACTTCACTCTCACCATCAGCAGACTGGAG |
| | | | CCTGAAGATTTTGCATTATATTACTGTCAGCAATATGATT |
| | | | CCTCGCCATTCACTTTCGGCCCTGGGACCAAAGTGGATAT |
| | | | CAAACGTACGGTAGCTGCCCCTTCAGTTTTTATCTTTCCG |
| | | | CCGTCTGACGAGCAGTTAAAATCCGGGACCGCTTCTGTAG |
| | | | TTTGCCTGCTGAATAATTTTTATCCGCGTGAGGCTAAAGT |
| | | | ACAATGGAAAGTCGACAATGCTTTGCAGTCGGGAAATTCA |
| | | | CAGGAAAGTGTTACGGAGCAGGATTCTAAAGATTCCACAT |
| | | | ATTCACTCAGCTCCACCCTTACACTGAGCAAAGCCGACTA |
| | | | TGAAAAACATAAAGTTTACGCATGTGAGGTGACGCACCAA |
| | | | GGATTATCCAGTCCGGTCACAAAATCGTTTAACCGCGGTG |
| | | | AGTGT |
| 1557 | ATX-P-3472 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTACCCGGGAATCCGGGGTCCCTGACCG |
| | | | ATTCAGTGGCAGCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1558 | ATX-P-3361 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1559 | ATX-P-3366 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1560 | ATX-P-3376 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1561 | ATX-P-3477 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTAACCGGGAATCCGGGGTCCCTGACCG |
| | | | ATTCAGTGGCTCCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCGTACACTTTTGGCCAGGG |
| | | | GACCAAGCTGGAGATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1562 | ATX-P-3418 | LC Kappa nt | GACATCCAGATGACCCAGTCTCCATCCTCCCTGTCTGCAT |
| | | | CTGTAGGAGACAGAGTCACCATCACCTGCCGGGCAAGTCA |
| | | | GACCATTAGAAACTTTTTAAATTGGTATCAGCGGAAACCA |
| | | | GGGAAAGCCCCTAAGATCCTGATCTATGCTGCATCCAGTT |
| | | | TGCAAAGTGGGGTCCCATCAAGGTTCAGTGGCAGTGGATC |
| | | | TGGGACAGATTTCACTCTCACCATCAGCAGTCTGCAACCT |
| | | | GCAGATTTTGCAACTTACTACTGTCAACAGACTTACAGTA |
| | | | TCCCGATCACCTTCGGCCAAGGGACACGACTGGAGATTAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1563 | ATX-P-3420 | LC Kappa nt | GACATCCAGATGACCCAGTCTCCATCCTCCCTGTCTGCAT |
| | | | CTGTAGGAGACAGAGTCACCATCACTTGCCGGGCAAGTCA |
| | | | GAGCATTAGCAGGTATTTAAATTGGTATCAGCAGAAACCA |
| | | | GGGAAAGCCCCTAAACTCCTGCTCTATGCTGCATCCAGTT |
| | | | TGCAAAGTGGGGTCCCATCAAAGTTCAGCGGCAGTGGATC |
| | | | TGGGACAGATTTCACTCTCACCATCAGCAGCCTGCAACCT |
| | | | GAAGATTTTGCAACTTACTACTGTCAACAGAGTTACAGTT |
| | | | CCCCGATCACCTTCGGCCAAGGGACACGACTGGAGATTAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1564 | ATX-P-3426 | LC Kappa nt | GATATTGTGATGACTCAGTCTCCACTCTCCCTGCCCGTCA |
| | | | CCCCTGGAGAGCCGGCCTCCATCTCCTGCAGGTCTAGTCA |
| | | | GAGCCTCCTGCATAGTAATGGATACAACTATTTGGATTGG |
| | | | TACCTGCAGAAGCCAGGGCAGTCTCCACAGCTCCTGATCT |
| | | | ATTTGGGTTCTAATCGGGCCTCCGGGGTCCCTGACAGGTT |
| | | | CAGTGGCAGTGGATCAGGCACAGATTTTACACTGAAAATC |
| | | | AGCAGAGTGGAGGCTGAGGATGTTGGGGTTTATTACTGCA |
| | | | TGCAAACTCTACAAACTCCTCCGACGTTCGGCCAAGGGAC |
| | | | CAAGGTGGAAATCAAACGTACGGTAGCTGCCCCTTCAGTT |
| | | | TTTATCTTTCCGCCGTCTGACGAGCAGTTAAAATCCGGGA |
| | | | CCGCTTCTGTAGTTTGCCTGCTGAATAATTTTTATCCGCG |
| | | | TGAGGCTAAAGTACAATGGAAAGTCGACAATGCTTTGCAG |
| | | | TCGGGAAATTCACAGGAAAGTGTTACGGAGCAGGATTCTA |
| | | | AAGATTCCACATATTCACTCAGCTCCACCCTTACACTGAG |
| | | | CAAAGCCGACTATGAAAAACATAAAGTTTACGCATGTGAG |
| | | | GTGACGCACCAAGGATTATCCAGTCCGGTCACAAAATCGT |
| | | | TTAACCGCGGTGAGTGT |
| 1565 | ATX-P-3432 | LC Kappa nt | GACATCCAGTTGACCCAGTCTCCATCCTTCCTGTCTGCAT |
| | | | CTGTAGGAGACAGACTCACCATCACTTGCCGGGCCAGTCA |
| | | | GGGCATTAGCAGTTATTTAGCCTGGTATCAGCAAAAACCA |
| | | | GGGAAAGCCCCTAAGCTCCTGATCTATACTGCATCCACTT |
| | | | TACAAAGTGGGGTCCCATCAAGGTTCAGCGGCAGTGGATC |
| | | | TGGGACAGAATTCACTCTCACAATCAGCAGCCTGCAGCCT |
| | | | GAAGATTTTGCAACTTATTACTGTCAACAGCTTAATGGTT |
| | | | ACCCTCCGACGTTCGGCCAAGGGACCAAGGTGGAAATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1566 | ATX-P-3433 | LC Kappa nt | GACATCCAGATGACCCAGTCTCCATCCTCCCTGTCTGCAT |
| | | | CTGTAGGAGACAGAGTCACCATCACTTGCCGGGCAAGTCA |
| | | | GAGCATTACCAGCTATTTAAATTGGTATCAGCAGAAACCA |
| | | | GGGAAAGCCCCTAAGCTCCTGATCTTTGCTGCATCCAGTT |
| | | | TGCAAAGTGGGGTCCCATCAAGGTTCAGTGGCAGTGGATC |
| | | | TGGGACAGATTTCACTCTCACCATCAGCAGTCTGCAACCT |
| | | | GAAGATTTTGCAACTTACTACTGTCAACAGAGTTACAGTA |
| | | | CCCCTCTCACTTTCGGCGGAGGGACCAAGGTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1567 | ATX-P-3438 | LC Kappa nt | GACATCGTGATGACCCAGTCTCCAGACTCCCTGGCTGTGT |
| | | | CTCTGGGCGAGAGGGCCACCATCAACTGCAAGTCCAGCCA |
| | | | GAGTGTTTTATACAGCTCCAACAATAAGAACTACTTAGCT |
| | | | TGGTACCAGCAGAAACCAGGACAGCCTCCTAAGCTGCTCA |
| | | | TTTACTGGGCATCTACCCGGGAATCCGGGGTCCCTGACCG |
| | | | ATTCAGTGGCAGCGGGTCTGGGACAGATTTCACTCTCACC |
| | | | ATCAGCAGCCTGCAGGCTGAAGATGTGGCAGTTTATTACT |
| | | | GTCAGCAATATTATAGTACTCCTCGGACGTTCGGCCAGGG |
| | | | GACCAAGGTGGAAATCAAACGTACGGTAGCTGCCCCTTCA |
| | | | GTTTTTATCTTTCCGCCGTCTGACGAGCAGTTAAAATCCG |
| | | | GGACCGCTTCTGTAGTTTGCCTGCTGAATAATTTTTATCC |
| | | | GCGTGAGGCTAAAGTACAATGGAAAGTCGACAATGCTTTG |
| | | | CAGTCGGGAAATTCACAGGAAAGTGTTACGGAGCAGGATT |
| | | | CTAAAGATTCCACATATTCACTCAGCTCCACCCTTACACT |
| | | | GAGCAAAGCCGACTATGAAAAACATAAAGTTTACGCATGT |
| | | | GAGGTGACGCACCAAGGATTATCCAGTCCGGTCACAAAAT |
| | | | CGTTTAACCGCGGTGAGTGT |
| 1568 | ATX-P-3441 | LC Kappa nt | GACATCCAGATGACCCAGTCTCCATCCTCCCTGTCTGCAT |
| | | | CTGTAGGAGAAAGAGTCACCATCACTTGCCGGGCAAGTCA |
| | | | GAGCATTAGCAGCTATTTAAATTGGTATCAGCAGAAACCA |
| | | | GGGAAAGCCCCTAACCTCCTGATCTATGCTGCATCCAGTT |
| | | | TCCAAAGTGGGGTCCCATCAAGGGTCAGTGGCAGTGGATC |
| | | | TGGGACAGATTTCACTCTCACCATCAGCAGTCTGCAACCT |
| | | | GAGGATTTTGCAACTTACTACTGTCAACAGAGTTACAGTA |
| | | | ACCCGTACACTTTTGGCCAGGGGACCAAGCTGGAGATCAA |
| | | | ACGTACGGTAGCTGCCCCTTCAGTTTTTATCTTTCCGCCG |
| | | | TCTGACGAGCAGTTAAAATCCGGGACCGCTTCTGTAGTTT |
| | | | GCCTGCTGAATAATTTTTATCCGCGTGAGGCTAAAGTACA |
| | | | ATGGAAAGTCGACAATGCTTTGCAGTCGGGAAATTCACAG |
| | | | GAAAGTGTTACGGAGCAGGATTCTAAAGATTCCACATATT |
| | | | CACTCAGCTCCACCCTTACACTGAGCAAAGCCGACTATGA |
| | | | AAAACATAAAGTTTACGCATGTGAGGTGACGCACCAAGGA |
| | | | TTATCCAGTCCGGTCACAAAATCGTTTAACCGCGGTGAGT |
| | | | GT |
| 1569 | ATX-P-3446 | LC Kappa nt | GAAATTGTGTTGACGCAGTCTCCAGGCACCCTGTCTTTGT |
| | | | CTCCAGGGGAAAGAGCCACCCTCTCCTGCAGGGCCAGTCA |
| | | | GAGTGTTAGTAGAAGCTACTTAGCCTGGTACCAGCAGAAA |
| | | | CCTGGCCAGGCTCCCAGGCTCCTCATCTATGGTGCATCCA |
| | | | GCAGGGCCACTGGCATCCCAGACAGGTTCAGTGGCAGTGG |
| | | | GTCTGGGACAGACTTCACTCTCACCATCAGCAGACTGGAG |
| | | | CCTGAAGATTTTGCAGTGTATTACTGTCAGCAGTATGGTA |
| | | | GCTCACCGTGGACGTTCGGCCAAGGGACCAAGGTGGAAAT |
| | | | CAAACGTACGGTAGCTGCCCCTTCAGTTTTTATCTTTCCG |
| | | | CCGTCTGACGAGCAGTTAAAATCCGGGACCGCTTCTGTAG |
| | | | TTTGCCTGCTGAATAATTTTTATCCGCGTGAGGCTAAAGT |
| | | | ACAATGGAAAGTCGACAATGCTTTGCAGTCGGGAAATTCA |
| | | | CAGGAAAGTGTTACGGAGCAGGATTCTAAAGATTCCACAT |
| | | | ATTCACTCAGCTCCACCCTTACACTGAGCAAAGCCGACTA |
| | | | TGAAAAACATAAAGTTTACGCATGTGAGGTGACGCACCAA |
| | | | GGATTATCCAGTCCGGTCACAAAATCGTTTAACCGCGGTG |
| | | | AGTGT |
| 1570 | ATX-P-3452 | LC Kappa nt | GATATTGTGATGACTCAGTCTCCACTCTCCCTGCCCGTCA |
| | | | CCCCTGGAGAGCCGGCCTCCATCTCCTGCAGGTCTAGTCA |
| | | | GATCCTCCTGCATACTAATGGATACAACTATTTGGATTGG |
| | | | TACCTGCAGAAGCCAGGGCAGTCTCCTCAGCTCCTGATCT |
| | | | ATTTGGGTTCTTATCGGGCCTCCGGGGTCCCTGACAGGTT |
| | | | CAGTGGCAGTGGATCAGGCACAGATTTTACACTGAAAATC |
| | | | AGCAGAGTGGAGGCTGGGGATGTTGGGCTTTATTACTGCA |
| | | | TGCAAGCTCTACAAACTCCGCTCACTTTCGGCGGAGGGAC |
| | | | CAAGGTGGAGATCAAACGTACGGTAGCTGCCCCTTCAGTT |
| | | | TTTATCTTTCCGCCGTCTGACGAGCAGTTAAAATCCGGGA |
| | | | CCGCTTCTGTAGTTTGCCTGCTGAATAATTTTTATCCGCG |
| | | | TGAGGCTAAAGTACAATGGAAAGTCGACAATGCTTTGCAG |
| | | | TCGGGAAATTCACAGGAAAGTGTTACGGAGCAGGATTCTA |
| | | | AAGATTCCACATATTCACTCAGCTCCACCCTTACACTGAG |
| | | | CAAAGCCGACTATGAAAAACATAAAGTTTACGCATGTGAG |
| | | | GTGACGCACCAAGGATTATCCAGTCCGGTCACAAAATCGT |
| | | | TTAACCGCGGTGAGTGT |
| 1571 | ATX-P-3360 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLILSKADYEKHKVYAC |
| | | | EVTHQGLSSPVTKSFNRGEC |
| 1572 | ATX-P-3362 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1573 | ATX-P-3363 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLILSKADYEKHKVYAC |
| | | | EVTHQGLSSPVTKSFNRGEC |
| 1574 | ATX-P-3365 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1575 | ATX-P-3367 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1576 | ATX-P-3369 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1577 | ATX-P-3370 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1578 | ATX-P-3371 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLILSKADYEKHKVYAC |
| | | | EVTHQGLSSPVTKSFNRGEC |
| 1579 | ATX-P-3373 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1580 | ATX-P-3375 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1581 | ATX-P-3378 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1582 | ATX-P-3382 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLILSKADYEKHKVYAC |
| | | | EVTHQGLSSPVTKSFNRGEC |
| 1583 | ATX-P-3383 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1584 | ATX-P-3384 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1585 | ATX-P-3386 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1586 | ATX-P-3387 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1587 | ATX-P-3389 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLILSKADYEKHKVYAC |
| | | | EVTHQGLSSPVTKSFNRGEC |
| 1588 | ATX-P-3391 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1589 | ATX-P-3392 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1590 | ATX-P-3395 | LC Kappa aa | DIQMTQSPSTLSASVGDSVTITCRASQNIITWLAWYQQKP |
| | | | GKAPNLLIYKASSLESGVPSRFSGSGSGTEFTLTISSLQP |
| | | | DDFATYYCQQYSSYSYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLILSKADYEKHKVYACEVTHQG |
| | | | LSSPVTKSFNRGEC |
| 1591 | ATX-P-3396 | LC Kappa aa | DIQMTQSPSTLSASVGDSVTITCRASQNIITWLAWYQQKP |
| | | | GKAPNLLIYKASSLESGVPSRFSGSGSGTEFTLTISSLQP |
| | | | DDFATYYCQQYSSYSYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1592 | ATX-P-3397 | LC Kappa aa | DIQMTQSPSTLSASVGDSVTITCRASQNIITWLAWYQQKP |
| | | | GKAPNLLIYKASSLESGVPSRFSGSGSGTEFTLTISSLQP |
| | | | DDFATYYCQQYSSYSYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1593 | ATX-P-3405 | LC Kappa aa | DIQMTQSPSTLSASVGDSVTITCRASQNIITWLAWYQQKP |
| | | | GKAPNLLIYKASSLESGVPSRFSGSGSGTEFTLTISSLQP |
| | | | DDFATYYCQQYSSYSYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1594 | ATX-P-3406 | LC Kappa aa | DIQMTQSPSTLSASVGDSVTITCRASQNIITWLAWYQQKP |
| | | | GKAPNLLIYKASSLESGVPSRFSGSGSGTEFTLTISSLQP |
| | | | DDFATYYCQQYSSYSYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1595 | ATX-P-3407 | LC Kappa aa | DIQMTQSPSTLSASVGDSVTITCRASQNIITWLAWYQQKP |
| | | | GKAPNLLIYKASSLESGVPSRFSGSGSGTEFTLTISSLQP |
| | | | DDFATYYCQQYSSYSYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1596 | ATX-P-3408 | LC Kappa aa | DIQMTQSPSTLSASVGDSVTITCRASQNIITWLAWYQQKP |
| | | | GKAPNLLIYKASSLESGVPSRFSGSGSGTEFTLTISSLQP |
| | | | DDFATYYCQQYSSYSYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1597 | ATX-P-3410 | LC Kappa aa | DIQMTQSPSTLSASVGDSVTITCRASQNIITWLAWYQQKP |
| | | | GKAPNLLIYKASSLESGVPSRFSGSGSGTEFTLTISSLQP |
| | | | DDFATYYCQQYSSYSYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLILSKADYEKHKVYACEVTHQG |
| | | | LSSPVTKSFNRGEC |
| 1598 | ATX-P-3412 | LC Kappa aa | DIQMTQSPSTLSASVGDSVTITCRASQNIITWLAWYQQKP |
| | | | GKAPNLLIYKASSLESGVPSRFSGSGSGTEFTLTISSLQP |
| | | | DDFATYYCQQYSSYSYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLILSKADYEKHKVYACEVTHQG |
| | | | LSSPVIKSENRGEC |
| 1599 | ATX-P-3413 | LC Kappa aa | DIQMTQSPSTLSASVGDSVTITCRASQNIITWLAWYQQKP |
| | | | GKAPNLLIYKASSLESGVPSRFSGSGSGTEFTLTISSLQP |
| | | | DDFATYYCQQYSSYSYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1600 | ATX-P-3414 | LC Kappa aa | DIQMTQSPSTLSASVGDSVTITCRASQNIITWLAWYQQKP |
| | | | GKAPNLLIYKASSLESGVPSRFSGSGSGTEFTLTISSLQP |
| | | | DDFATYYCQQYSSYSYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSENRGEC |
| 1601 | ATX-P-3419 | LC Kappa aa | AIQMTQSPSSLSASVGDRVTITCRASQGIRNDLGWYQQKP |
| | | | GKAPKLLIYAASSLQSGVPSRFSGSGSGTDFTLTISSLQP |
| | | | EDFATYYCLQDYNYPWTFGQGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1602 | ATX-P-3421 | LC Kappa aa | DIVMTQSPDSLAVSLGERATINCKSSQTVLYRSNNKNYLA |
| | | | WYQQKPGQPPKLLIYWASTRESGVPDRFSGSGSGTDFTLT |
| | | | ISSLQAEDVAVYYCQQYYSTPWTFGQGTKVEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1603 | ATX-P-3428 | LC Kappa aa | AIQMTQSPSSLSASVGDRVTITCRASQGIRNDLGWYQQNP |
| | | | GKAPKLLIYAASNLQSGVPSRFSGSGSGTDFTLTISSLQP |
| | | | EDFVTYYCLQDYNWPWTFGQGTRLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVIKSFNRGEC |
| 1604 | ATX-P-3430 | LC Kappa aa | DIQMTQSPSSLSASVGDRVTITCRASQSISNYLNWYQQKP |
| | | | EKAPKFLIYAASSLQSGVPSRFSGSGSGTDFTLTISSLQP |
| | | | EDFATYYCQQSYSSPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1605 | ATX-P-3431 | LC Kappa aa | EIVLTQSPDFQSVTPKEKVTITCRASQSIGSNLHWFQQKP |
| | | | DQSPKLLIKYASQSFSGVPSRFSGSGSGTDFTLTINSLEA |
| | | | EDAAMYYCHQSSSLPRTFGQGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVIKSFNRGEC |
| 1606 | ATX-P-3435 | LC Kappa aa | AIQMTQSPSSLSASVGDRVTITCRASQGIRNDLGWYQQKP |
| | | | GKAPKLLIYAASSLQSGVPSRFSGSGSGTDFTLTISSLQP |
| | | | EDFASYYCLQDYNWPYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1607 | ATX-P-3436 | LC Kappa aa | EIVLTQSPGTLSLSPGERATLSCRASQSVRSSYFAWYQQK |
| | | | PGQAPRLLIYGASSRATGIPDRFSGSGSGTDFTLTISRLE |
| | | | PEDFAVYYCQQYGSSPRTFGQGTKVEIKRTVAAPSVFIFP |
| | | | PSDEQLKSGTASVVCLLNNFYPREAKVQWKVDNALQSGNS |
| | | | QESVTEQDSKDSTYSLSSTLTLSKADYEKHKVYACEVTHQ |
| | | | GLSSPVTKSFNRGEC |
| 1608 | ATX-P-3437 | LC Kappa aa | DIVMTQSPDSLAVSLGERATINCKSSQSVLYSSNNKNYLA |
| | | | WYQQKPGQPPKLLIYWASTRESGVPDRFSGSGSGTDFTLT |
| | | | ISSLQAEDVAVYYCQQYFGTPRTFGQGTKVEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1609 | ATX-P-3440 | LC Kappa aa | EIVLTQSPATLSLSPGERATLSCRASQSVSSYLAWYQQKP |
| | | | GQAPRLLIYDASNRATGIPARFSGSGSGTDFTLTISSLQS |
| | | | EDFAVYYCQQRSNWPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1610 | ATX-P-3442 | LC Kappa aa | DIQMTQSPSSLSASVGDRVTITCRASQTIRSYLNWYQQKP |
| | | | GKAPKLLIYAASSLQSGVPSRFSGSGSGTNFTLTISSLQP |
| | | | EDFATYSCQQSYTTPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1611 | ATX-P-3444 | LC Kappa aa | EIVLTQSPGTLSLSPGERATLSCRASQSISSSYLAWYQRE |
| | | | PGQAPRLLIYGASSRATGIPDRFSGSGSGTDFTLTISRLE |
| | | | PEDFVVYYCQQYGSSPITFGQGTRLEIKRTVAAPSVFIFP |
| | | | PSDEQLKSGTASVVCLLNNFYPREAKVQWKVDNALQSGNS |
| | | | QESVTEQDSKDSTYSLSSTLTLSKADYEKHKVYACEVTHQ |
| | | | GLSSPVTKSFNRGEC |
| 1612 | ATX-P-3447 | LC Kappa aa | EIVLTQSPGTLSLSPGERATLSCRASQSVRSSYLAWYQQK |
| | | | PGQAPRLLIYGASSRATGIPDRFSGSGSGTDFTLTFSRLE |
| | | | PEDFAVYYCQQYGSSPYTFGQGTKLEIKRTVAAPSVFIFP |
| | | | PSDEQLKSGTASVVCLLNNFYPREAKVQWKVDNALQSGNS |
| | | | QESVTEQDSKDSTYSLSSTLILSKADYEKHKVYACEVTHQ |
| | | | GLSSPVTKSFNRGEC |
| 1613 | ATX-P-3448 | LC Kappa aa | DIQMTQSPSSLSASVGDRVTITCQASQDISNYLNWYQQKP |
| | | | GKAPKLLIYDASNLETGVPSRFSGSGSGTDFTFTISSLQP |
| | | | EDFATYYCQQANSFPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1614 | ATX-P-3449 | LC Kappa aa | EIVLTQSPGTLSLSPGERATLSCRASQSVSSSYLAWYQQK |
| | | | PGQAPRLLIYGASSRATGIPDRFSGSGSGTDFTLTISRLE |
| | | | PEDFAVYYCQQYGSSPFTFGPGTKVDIKRTVAAPSVFIFP |
| | | | PSDEQLKSGTASVVCLLNNFYPREAKVQWKVDNALQSGNS |
| | | | QESVTEQDSKDSTYSLSSTLTLSKADYEKHKVYACEVTHQ |
| | | | GLSSPVTKSFNRGEC |
| 1615 | ATX-P-3450 | LC Kappa aa | DIVMTQSPDSLAVSLGERATINCKSSQSVLYSSNNKNYLA |
| | | | WYQQKPGQPPKLLIYWASTRESGVPDRFSGSGSGTDFTLT |
| | | | ISSLQAEDVAVYYCQQYYSTPWTFGQGTRLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1616 | ATX-P-3451 | LC Kappa aa | DIQMTQSPSSLSASVGDRVTITCRASQDISNYLNWYQQKP |
| | | | GKAPKLLIYAASSLESGVPSRFSGSGSGTDFTLTISSLQP |
| | | | EDFATYYCQQSYSTPTFGGGTKVEIKRTVAAPSVFIFPPS |
| | | | DEQLKSGTASVVCLLNNFYPREAKVQWKVDNALQSGNSQE |
| | | | SVTEQDSKDSTYSLSSTLTLSKADYEKHKVYACEVTHQGL |
| | | | SSPVTKSENRGEC |
| 1617 | ATX-P-3453 | LC Kappa aa | DIVMTQSPDSLAVSLGERATINCKSSQSVLYRSNNKNYLA |
| | | | WYQQKPGQPPKLLIYWASTRESGVPDRFSGSGSGTDFTLT |
| | | | ISSLQAEDVAVYYCQQYYGTPRTFGQGTKVEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1618 | ATX-P-3454 | LC Kappa aa | DIQMTQSPSSLSASVGDRVTITCQASQDIKNCLNWYQQKP |
| | | | GKAPKLLIYDASNLETGVPSRFSGSGSGTDFTFTISSLQP |
| | | | EDIATYYCQQFDNLPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSENRGEC |
| 1619 | ATX-P-3455 | LC Kappa aa | DIVMTQSPDSLAVSLGERATINCKSSQTILYRSNIKNYLA |
| | | | WYQQKPGQPPKLLISWASTRESGVPDRFSGSGSGTDFTLT |
| | | | ISSLQAEDVAVYYCQQYYSTRTFGQGTKLEIKRTVAAPSV |
| | | | FIFPPSDEQLKSGTASVVCLLNNFYPREAKVQWKVDNALQ |
| | | | SGNSQESVTEQDSKDSTYSLSSTLTLSKADYEKHKVYACE |
| | | | VTHQGLSSPVTKSFNRGEC |
| 1620 | ATX-P-3456 | LC Kappa aa | DIVMTQSPDSLAVSLGERATINCKSSQSVLYSSNNKNYLA |
| | | | WYQQKPGQPPKLLIYWASTRESGVPDRFSGSGSGTDFTLT |
| | | | ISSLQAEDVAVYYCQQYYSTPRTFGQGTKVEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1621 | ATX-P-3458 | LC Kappa aa | DIQMTQSPSTLSASVGDRVTITCRASQGISSWLAWYQQKP |
| | | | GKAPKLLIYAASSLQSGVPSRFSGSGSGTDFTLTISSLQP |
| | | | EDFATYYCQQANSFPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1622 | ATX-P-3459 | LC Kappa aa | DIVMTQSPDSLAVSLGERATINCKSSQSVLYSSNNKNYLA |
| | | | WYQQKPGQPPKLLIYWASTRESGVPDRFSGSGSGTDFTLT |
| | | | ISSLQAEDVAVYYCQQYYSTPPTFGQGTKVEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1623 | ATX-P-3462 | LC Kappa aa | DIQMTQSPSSLSASVGDRVTITCRASQSISSYLNWYQQKP |
| | | | GKAPKLLIYAASSLQSGVPSRFSGSGSGTDFTLTISSLQP |
| | | | EDFATYYCQQSYSTPYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1624 | ATX-P-3463 | LC Kappa aa | DIVMTQSPDSLAVSLGERATINCKSSQSVLYSSNNKNYLA |
| | | | WYQQKPGQPPKLLIYWASTRESGVPDRFSGSGSGTDFTLT |
| | | | ISSLQAEDVAVYYCQQFYDTPRTFGQGTKVEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1625 | ATX-P-3466 | LC Kappa aa | DIQMTQSPSSLSASVGDRVTITCRASQGISNYLAWYQQKP |
| | | | GKVPKLLIYVASTLQSGVPSRFSGSGSGTDFTLTISSLQP |
| | | | EDVATYYCQNYISAPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSENRGEC |
| 1626 | ATX-P-3467 | LC Kappa aa | DIVMTQSPDSLAVSLGERATINCKSSQSVLYSSNNKNYLA |
| | | | WYQQKPGQPPKLLIYWASTRESGVPDRFSGSGSGTDFTLT |
| | | | ISSLQAEDVAVYYCQQYYSTPWTFGQGTKVEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1627 | ATX-P-3468 | LC Kappa aa | EIVLTQSPGTLSLSPGERATLSCRASQSVSSSYLAWYQQK |
| | | | PGQAPRLLIYGASSRATGIPDRFSGSGSGTDFTLTISRLE |
| | | | PEDFAVYYCQQYGSSPYTFGQGTKLEIKRTVAAPSVFIFP |
| | | | PSDEQLKSGTASVVCLLNNFYPREAKVQWKVDNALQSGNS |
| | | | QESVTEQDSKDSTYSLSSTLTLSKADYEKHKVYACEVTHQ |
| | | | GLSSPVTKSFNRGEC |
| 1628 | ATX-P-3469 | LC Kappa aa | DIQMTQSPSSLSASVGDRVNITCRASQGISNYLAWYQQKP |
| | | | GKVPKLLIYAASTLQSGVPSRFSGSGSGTDFTLTISSLQP |
| | | | EDVASYFCQKYISAPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSENRGEC |
| 1629 | ATX-P-3470 | LC Kappa aa | DIQMTQSPSSLSASVGDRVTITCRASQGISNYLAWYQQKP |
| | | | GKVPKLLIYAASTLQSGVPSRFSGSGSGTDFTLTISSLQP |
| | | | EDVASYYCQDYTSAPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSENRGEC |
| 1630 | ATX-P-3471 | LC Kappa aa | EIVLTQSPGTLSLSPGERATLSCRASQSVSDSYLAWYQQE |
| | | | PGQAPRLLIYGASSRATGIPDRFSGSGSGTDFTLTISRLE |
| | | | PEDFALYYCQQYDSSPFTFGPGTKVDIKRTVAAPSVFIFP |
| | | | PSDEQLKSGTASVVCLLNNFYPREAKVQWKVDNALQSGNS |
| | | | QESVTEQDSKDSTYSLSSTLTLSKADYEKHKVYACEVTHQ |
| | | | GLSSPVTKSFNRGEC |
| 1631 | ATX-P-3472 | LC Kappa aa | DIVMTQSPDSLAVSLGERATINCKSSQSVLYSSNNKNYLA |
| | | | WYQQKPGQPPKLLIYWASTRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1632 | ATX-P-3361 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1633 | ATX-P-3366 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1634 | ATX-P-3376 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1635 | ATX-P-3377 | LC Kappa aa | DIVMTQSPDSLAVSLGERATINCKSSQSVLYSSNNKNYLA |
| | | | WYQQKPGQPPKLLIYWASNRESGVPDRFSGSGSGTDFTLT |
| | | | ISSLQAEDVAVYYCQQYYSTPYTFGQGTKLEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1636 | ATX-P-3418 | LC Kappa aa | DIQMTQSPSSLSASVGDRVTITCRASQTIRNFLNWYQRKP |
| | | | GKAPKILIYAASSLQSGVPSRFSGSGSGTDFTLTISSLQP |
| | | | ADFATYYCQQTYSIPITFGQGTRLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1637 | ATX-P-3420 | LC Kappa aa | DIQMTQSPSSLSASVGDRVTITCRASQSISRYLNWYQQKP |
| | | | GKAPKLLLYAASSLQSGVPSKFSGSGSGTDFTLTISSLQP |
| | | | EDFATYYCQQSYSSPITFGQGTRLEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSENRGEC |
| 1638 | ATX-P-3426 | LC Kappa aa | DIVMTQSPLSLPVTPGEPASISCRSSQSLLHSNGYNYLDW |
| | | | YLQKPGQSPQLLIYLGSNRASGVPDRFSGSGSGTDFTLKI |
| | | | SRVEAEDVGVYYCMQTLQTPPTFGQGTKVEIKRTVAAPSV |
| | | | FIFPPSDEQLKSGTASVVCLLNNFYPREAKVQWKVDNALQ |
| | | | SGNSQESVTEQDSKDSTYSLSSTLTLSKADYEKHKVYACE |
| | | | VTHQGLSSPVTKSFNRGEC |
| 1639 | ATX-P-3432 | LC Kappa aa | DIQLTQSPSFLSASVGDRLTITCRASQGISSYLAWYQQKP |
| | | | GKAPKLLIYTASTLQSGVPSRFSGSGSGTEFTLTISSLQP |
| | | | EDFATYYCQQLNGYPPTFGQGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1640 | ATX-P-3433 | LC Kappa aa | DIQMTQSPSSLSASVGDRVTITCRASQSITSYLNWYQQKP |
| | | | GKAPKLLIFAASSLQSGVPSRFSGSGSGTDFTLTISSLQP |
| | | | EDFATYYCQQSYSTPLTFGGGTKVEIKRTVAAPSVFIFPP |
| | | | SDEQLKSGTASVVCLLNNFYPREAKVQWKVDNALQSGNSQ |
| | | | ESVTEQDSKDSTYSLSSTLTLSKADYEKHKVYACEVTHQG |
| | | | LSSPVTKSFNRGEC |
| 1641 | ATX-P-3438 | LC Kappa aa | DIVMTQSPDSLAVSLGERATINCKSSQSVLYSSNNKNYLA |
| | | | WYQQKPGQPPKLLIYWASTRESGVPDRFSGSGSGTDFTLT |
| | | | ISSLQAEDVAVYYCQQYYSTPRTFGQGTKVEIKRTVAAPS |
| | | | VFIFPPSDEQLKSGTASVVCLLNNFYPREAKVQWKVDNAL |
| | | | QSGNSQESVTEQDSKDSTYSLSSTLTLSKADYEKHKVYAC |
| | | | EVTHQGLSSPVTKSFNRGEC |
| 1642 | ATX-P-3441 | LC Kappa aa | DIQMTQSPSSLSASVGERVTITCRASQSISSYLNWYQQKP |
| | | | GKAPNLLIYAASSFQSGVPSRVSGSGSGTDFTLTISSLQP |
| | | | EDFATYYCQQSYSNPYTFGQGTKLEIKRTVAAPSVFIFPP |
| | | | SDEQLKSGTASVVCLLNNFYPREAKVQWKVDNALQSGNSQ |
| | | | ESVTEQDSKDSTYSLSSTLILSKADYEKHKVYACEVTHQG |
| | | | LSSPVTKSFNRGEC |
| 1643 | ATX-P-3446 | LC Kappa aa | EIVLTQSPGTLSLSPGERATLSCRASQSVSRSYLAWYQQK |
| | | | PGQAPRLLIYGASSRATGIPDRFSGSGSGTDFTLTISRLE |
| | | | PEDFAVYYCQQYGSSPWTFGQGTKVEIKRTVAAPSVFIFP |
| | | | PSDEQLKSGTASVVCLLNNFYPREAKVQWKVDNALQSGNS |
| | | | QESVTEQDSKDSTYSLSSTLTLSKADYEKHKVYACEVTHQ |
| | | | GLSSPVTKSFNRGEC |
| 1644 | ATX-P-3452 | LC Kappa aa | DIVMTQSPLSLPVTPGEPASISCRSSQILLHTNGYNYLDW |
| | | | YLQKPGQSPQLLIYLGSYRASGVPDRFSGSGSGTDFTLKI |
| | | | SRVEAGDVGLYYCMQALQTPLTFGGGTKVEIKRTVAAPSV |
| | | | FIFPPSDEQLKSGTASVVCLLNNFYPREAKVQWKVDNALQ |
| | | | SGNSQESVTEQDSKDSTYSLSSTLTLSKADYEKHKVYACE |
| | | | VTHQGLSSPVTKSENRGEC |

Human Complement Component 3 (C3), which includes Complement Component 3a (C3a) and Complement Component 3b (C3b), was used to generate the anti-C3 antibodies of the present disclosure (see, e.g., UniProt Accession No.

(SEQ ID NO: 1645)
M0QYC8): ITHRIHWESASLLRSEETKENEGFTVTAEGKGQGTLSV
VTMYHAKAKDQLTCNKFDLKVTIKPAPETGIPSPIFLSSVFLEKRPQ
DAKNTMILEICTRYRGDQDATMSILDISMMTGFAPDTDDLKQLANGV
DRYISKYELDKAFSDRNTLIIYLDKVSHSEDDCLAFKVHQYFNVELI
QPGAVKVYAYYNL.

Various embodiments of the present disclosure are described herein. Variations of those embodiments may become apparent to those of ordinary skill in the art upon reading the foregoing description. The inventors expect skilled artisans to employ such variations as appropriate, and the inventors intend for the various embodiments of the present disclosure to be practiced otherwise than as specifically described herein. Accordingly, embodiments of the present disclosure include all modifications and equivalents of the subject matter recited in the claims appended hereto as permitted by applicable law. Moreover, any combination of the above-described elements in all possible variations thereof is encompassed by the various embodiments of the present disclosure unless otherwise indicated herein or otherwise clearly contradicted by context.

What is claimed is:

1. A bispecific antibody, or antigen binding fragment thereof, which specifically binds human Complement Factor H Related 4 (CFHR) and human Complement Component 3 (C3), optionally wherein said human CFHR4 is the CFHR4 b variant (CFHR4b), which is optionally a polypeptide comprising or consisting of the amino acid sequence of SEQ ID NO: 580, and optionally wherein said human C3 is the b variant of C3 (C3b), which is optionally a polypeptide comprising or consisting of the amino acid sequence of SEQ ID NO: 1645.

2. The bispecific antibody of fragment of claim 1, which is monoclonal, optionally recombinant.

3. The bispecific antibody or fragment according to any one of the preceding claims, which is human, humanized or chimeric.

4. The bispecific antibody or fragment according to any one of the preceding claims, which is a full length antibody, a single chain antibody, a single chain variable fragment (scFv), a variable fragment (Fv), a fragment antigen-binding region (Fab), a Fab-C, a Fab′-SH, a (Fab′)2, a single-domain antibody (sdAb), a VHH antibody, a nanobody, a camelid-derived single-domain antibody, a shark IgNAR-derived single-domain antibody fragment (VNAR), a diabody, a triabody, an anticalin or an aptamer, optionally wherein the antibody is a full length antibody comprising an Fc region such as a human IgG1, IgG2, IgG3 or IgG4 region.

5. The bispecific antibody or fragment according to any one of the preceding claims, which is conjugated to at least one additional moiety, optionally selected from:
a. an antigen binding moiety, such as an antibody or antigen-binding fragment thereof, which is capable of specific binding to a target which is not human CFHR4 or human C3, preferably wherein said target is expressed in the human eye;
b. a therapeutic or cytotoxic moiety;
c. a detection moiety;
d. a purification moiety;
e. a half-life extension moiety, optionally a polypeptide that is at least 20 amino acids in length and comprises any combination of G, A, S T, E, and P residue, which polypeptide is conjugated to the C- or N-terminus of the antibody.

6. The bispecific antibody or fragment according to any one of the preceding claims, which is a polypeptide comprising:
a. one, two or all three HCDRs of any one of the exemplary CFHR4 antibodies the sequences of which are provided in Table 3; and one, two or all three of the corresponding LCDRs of said exemplary antibody; and/or
b. a VH sequence having at least 90% identity to the VH sequence of any one of the exemplary CFHR4 antibodies the sequences of which are provided in Table 4; and optionally a VL sequence having at least 90% identity to the corresponding VL sequence of said exemplary antibody, preferably wherein variation is not permitted in the HCDRs or LCDRs; and/or
c. all six CDRs of any one of the exemplary CFHR4 antibodies the sequences of which are provided in Table 3;
d. the VH and VL sequences of any one of the exemplary CFHR4 antibodies the sequences of which are provided in Table 4; and/or
e. the full length heavy chain (VH+constant) sequence of any one of the exemplary CFHR4 antibodies the sequences of which are provided in Table 5; and optionally the corresponding full length light chain (VL+constant) sequence of said exemplary antibody.

7. The bispecific antibody or fragment according to any one of the preceding claims, which is a polypeptide comprising:
a. one, two or all three HCDRs of any one of the exemplary C3 antibodies the sequences of which are provided in Table 6; and one, two or all three of the corresponding LCDRs of said exemplary antibody; and/or
b. a VH sequence having at least 90% identity to the VH sequence of any one of the exemplary C3 antibodies the sequences of which are provided in Table 7; and optionally a VL sequence having at least 90% identity to the corresponding VL sequence of said exemplary antibody provided in Table 8, preferably wherein variation is not permitted in the HCDRs or LCDRs; and/or
c. all six CDRs of any one of the exemplary C3 antibodies the sequences of which are provided in Table 6;
d. the VH and VL sequences of any one of the exemplary C3 antibodies the sequences of which are provided in Tables 7 and 8; and/or
e. the full length heavy chain (VH+constant) sequence of any one of the exemplary C3 antibodies the sequences of which are provided in Table 9; and optionally the corresponding full length light chain (VL+constant) sequence of said exemplary antibody provided in Table 10.

8. A polynucleotide encoding an antibody or fragment according to any one of the preceding claims, optionally wherein said polynucleotide comprises or consists of a nucleic acid sequence having at least 70, 80, 90 or 100% identity to a nucleic acid sequence of any one of the exemplary antibodies the sequences of which are provided in Tables 5, 9, and 10.

9. An expression vector comprising the polynucleotide of claim 8, which is optionally an adeno-associated virus (AAV) vector, a lentiviral (LV) vector, a herpes simplex virus (HSV) vector, or a retrovirus vector.

10. A pharmaceutical composition comprising an antibody or fragment, a polynucleotide, or a vector according to any one of the preceding claims, and optionally
a. at least one pharmaceutically acceptable carrier, diluent or preservative; and/or
b. at least one additional active ingredient.

11. The pharmaceutical composition of claim 10, which is suitable for ocular administration to a subject, optionally by delivery using a conjunctival insert, a contact lens, a gel, a nanoparticle, a mucoadhesive polymer, an ointment, a solution, a suspension, eye drops, and/or an implant, preferably by injection into the vitreous fluid.

12. The antibody or fragment according to any one of claims 1 to 7, the polynucleotide of claim 8, the vector of claim 9, or the composition of claim 10 or 11, for use as a medicament, optionally for use in a method of treating a disease of the eye in a subject.

13. The antibody, fragment, polynucleotide, vector or composition for use according to claim 12, wherein said disease is characterized by increased activation of the complement system, in particular the alternative pathway, and in particular within the eye of the subject, for example in drusen or retinal pigment epithelium (RPE) cells of the subject.

14. The antibody, fragment, polynucleotide, vector or composition for use according to claim 12 or 13, wherein the method comprises ocular administration of the antibody, preferably by injection into the vitreous fluid, and wherein said administration preferably relieves at least one symptom in the subject selected from visual distortion, reduced central vision, blurred vision, and/or difficulty adapting to low light.

15. The antibody, fragment, polynucleotide, vector or composition for use according to any one of claims 12 to 14, where the disease is age-related macular degeneration (AMD), optionally wherein said AMD is dry AMD, which may be at an early, intermediate or advanced stage (the latter otherwise known as geographic atrophy, GA).

16. A bispecific antibody comprising a first antigen-binding site directed against Complement Factor H Related 4 (CFHR4) and a second antigen-binding site directed against Complement Component 3 (C3):
wherein the first anti-CFHR4 antigen-binding site comprises a heavy chain variable region (VH) comprising complementarity determining regions (CDRs) HCDR1, HCDR2, and HCDR3 from at least one of SEQ ID NOs: 260-265, 284-293, 324-334, and 368-380; and a light chain variable region (VL) comprising complementarity determining regions (CDRs) LCDR1, LCDR2, and LCDR3 from at least one of SEQ ID NOs: 272-277, 304-313, 346-356, and 394-406; and
wherein the second anti-C3 antigen-binding site comprises a heavy chain variable region (VH) comprising complementarity determining regions (CDRs) HCDR1, HCDR2, and HCDR3; and a light chain variable region (VL) comprising complementarity determining regions (CDRs) LCDR1, LCDR2, and LCDR3.

17. A bispecific antibody comprising a first antigen-binding site directed against Complement Factor H Related 4 (CFHR4) and a second antigen-binding site directed against Complement Component 3 (C3):
wherein the first anti-CFHR4 antigen-binding site comprises a heavy chain variable region (VH) comprising complementarity determining regions (CDRs) HCDR1, HCDR2, and HCDR3; and a light chain variable region (VL) comprising complementarity determining regions (CDRs) LCDR1, LCDR2, and LCDR3; and
wherein the second anti-C3 antigen-binding site comprises a heavy chain variable region (VH) comprising complementarity determining regions (CDRs) HCDR1, HCDR2, and HCDR3 from at least one of SEQ ID NOs: 1127-1200; and a light chain variable region (VL) comprising complementarity determining regions (CDRs) LCDR1, LCDR2, and LCDR3 from at least one of SEQ ID NOs: 1275-1348.

18. A bispecific antibody comprising a first antigen-binding site directed against Complement Factor H Related 4 (CFHR4) and a second antigen-binding site directed against Complement Component 3 (C3):
wherein the first anti-CFHR4 antigen-binding site comprises a heavy chain variable region (VH) comprising complementarity determining regions (CDRs) HCDR1, HCDR2, and HCDR3 from at least one of SEQ ID NOs: 260-265, 284-293, 324-334, and 368-380; and a light chain variable region (VL) comprising complementarity determining regions (CDRs) LCDR1, LCDR2, and LCDR3 from at least one of SEQ ID NOs: 272-277, 304-313, 346-356, and 394-406; and
wherein the second anti-C3 antigen-binding site comprises a heavy chain variable region (VH) comprising complementarity determining regions (CDRs) HCDR1, HCDR2, and HCDR3 from at least one of SEQ ID NOs: 1127-1200; and a light chain variable region (VL) comprising complementarity determining regions (CDRs) LCDR1, LCDR2, and LCDR3 from at least one of SEQ ID NOs: 1275-1348.

19. The bispecific antibody of any one of claims 16 to 18, wherein the anti-CFHR4 HCDR1 comprises the amino acid sequence of SEQ ID NO: 2; the anti-CFHR4 HCDR2 comprises the amino acid sequence of SEQ ID NO: 9; and the anti-CFHR4 HCDR3 comprises the amino acid sequence of SEQ ID NO: 15.

20. The bispecific antibody of any one of claims 16 to 18, wherein the anti-CFHR4 HCDR1 comprises the amino acid sequence of SEQ ID NO: 3; the anti-CFHR4 HCDR2 comprises the amino acid sequence of SEQ ID NO: 10; and the anti-CFHR4 HCDR3 comprises the amino acid sequence of SEQ ID NO: 16.

21. The bispecific antibody of any one of claims 16 to 18, wherein the anti-CFHR4 HCDR1 comprises the amino acid sequence of SEQ ID NO: 4; the anti-CFHR4 HCDR2 comprises the amino acid sequence of SEQ ID NO: 11; and the anti-CFHR4 HCDR3 comprises the amino acid sequence of SEQ ID NO: 17.

22. The bispecific antibody of any one of claims 16 to 18, wherein the anti-CFHR4 HCDR1 comprises the amino acid sequence of SEQ ID NO: 5; the anti-CFHR4 HCDR2 comprises the amino acid sequence of SEQ ID NO: 12; and the anti-CFHR4 HCDR3 comprises the amino acid sequence of SEQ ID NO: 18.

23. The bispecific antibody of any one of claims 16 to 18, wherein the anti-CFHR4 HCDR1 comprises the amino acid sequence of SEQ ID NO: 6; the anti-CFHR4 HCDR2 comprises the amino acid sequence of SEQ ID NO: 13; and the anti-CFHR4 HCDR3 comprises the amino acid sequence of SEQ ID NO: 19.

24. The bispecific antibody of any one of claims 16 to 18, wherein the anti-CFHR4 HCDR1 comprises the amino acid sequence of SEQ ID NO: 7; the anti-CFHR4 HCDR2 comprises the amino acid sequence of SEQ ID NO: 14; and the anti-CFHR4 HCDR3 comprises the amino acid sequence of SEQ ID NO: 20.

25. The bispecific antibody of any one of claims 16 to 18, wherein the anti-CFHR4 HCDR1 comprises the amino acid sequence of SEQ ID NO: 22; the anti-CFHR4 HCDR2 comprises the amino acid sequence of SEQ ID NO: 33; and the anti-CFHR4 HCDR3 comprises the amino acid sequence of SEQ ID NO: 43.

26. The bispecific antibody of any one of claims 16 to 18, wherein the anti-CFHR4 HCDR1 comprises the amino acid sequence of SEQ ID NO: 23; the anti-CFHR4 HCDR2 comprises the amino acid sequence of SEQ ID NO: 34; and the anti-CFHR4 HCDR3 comprises the amino acid sequence of SEQ ID NO: 44.

27. The bispecific antibody of any one of claims 16 to 18, wherein the anti-CFHR4 HCDR1 comprises the amino acid sequence of SEQ ID NO: 24; the anti-CFHR4 HCDR2 comprises the amino acid sequence of SEQ ID NO: 35; and the anti-CFHR4 HCDR3 comprises the amino acid sequence of SEQ ID NO: 45.

28. The bispecific antibody of any one of claims 16 to 18, wherein the anti-CFHR4 HCDR1 comprises the amino acid sequence of SEQ ID NO: 25; the anti-CFHR4 HCDR2 comprises the amino acid sequence of SEQ ID NO: 36; and the anti-CFHR4 HCDR3 comprises the amino acid sequence of SEQ ID NO: 46.

29. The bispecific antibody of any one of claims 16 to 18, wherein the anti-CFHR4 HCDR1 comprises the amino acid sequence of SEQ ID NO: 26; the anti-CFHR4 HCDR2 comprises the amino acid sequence of SEQ ID NO: 37; and the anti-CFHR4 HCDR3 comprises the amino acid sequence of SEQ ID NO: 47.

30. The bispecific antibody of any one of claims 16 to 18, wherein the anti-CFHR4 HCDR1 comprises the amino acid sequence of SEQ ID NO: 27; the anti-CFHR4 HCDR2 comprises the amino acid sequence of SEQ ID NO: 38; and the anti-CFHR4 HCDR3 comprises the amino acid sequence of SEQ ID NO: 48.

31. The bispecific antibody of any one of claims 16 to 18, wherein the anti-CFHR4 HCDR1 comprises the amino acid sequence of SEQ ID NO: 28; the anti-CFHR4 HCDR2 comprises the amino acid sequence of SEQ ID NO: 39; and the anti-CFHR4 HCDR3 comprises the amino acid sequence of SEQ ID NO: 49.

32. The bispecific antibody of any one of claims 16 to 18, wherein the anti-CFHR4 HCDR1 comprises the amino acid sequence of SEQ ID NO: 29; the anti-CFHR4 HCDR2 comprises the amino acid sequence of SEQ ID NO: 40; and the anti-CFHR4 HCDR3 comprises the amino acid sequence of SEQ ID NO: 50.

33. The bispecific antibody of any one of claims 16 to 18, wherein the anti-CFHR4 HCDR1 comprises the amino acid sequence of SEQ ID NO: 30; the anti-CFHR4 HCDR2 comprises the amino acid sequence of SEQ ID NO: 41; and the anti-CFHR4 HCDR3 comprises the amino acid sequence of SEQ ID NO: 51.

34. The bispecific antibody of any one of claims 16 to 18, wherein the anti-CFHR4 HCDR1 comprises the amino acid sequence of SEQ ID NO: 31; the anti-CFHR4 HCDR2 comprises the amino acid sequence of SEQ ID NO: 42; and the anti-CFHR4 HCDR3 comprises the amino acid sequence of SEQ ID NO: 52.

35. The bispecific antibody of any one of claims 16 to 18, wherein the anti-CFHR4 HCDR1 comprises the amino acid sequence of SEQ ID NO: 54; the anti-CFHR4 HCDR2 comprises the amino acid sequence of SEQ ID NO: 66; and the anti-CFHR4 HCDR3 comprises the amino acid sequence of SEQ ID NO: 77.

36. The bispecific antibody of any one of claims 16 to 18, wherein the anti-CFHR4 HCDR1 comprises the amino acid sequence of SEQ ID NO: 55; the anti-CFHR4 HCDR2 comprises the amino acid sequence of SEQ ID NO: 67; and the anti-CFHR4 HCDR3 comprises the amino acid sequence of SEQ ID NO: 78.

37. The bispecific antibody of any one of claims 16 to 18, wherein the anti-CFHR4 HCDR1 comprises the amino acid sequence of SEQ ID NO: 56; the anti-CFHR4 HCDR2 comprises the amino acid sequence of SEQ ID NO: 68; and the anti-CFHR4 HCDR3 comprises the amino acid sequence of SEQ ID NO: 79.

38. The bispecific antibody of any one of claims 16 to 18, wherein the anti-CFHR4 HCDR1 comprises the amino acid sequence of SEQ ID NO: 57; the anti-CFHR4 HCDR2 comprises the amino acid sequence of SEQ ID NO: 69; and the anti-CFHR4 HCDR3 comprises the amino acid sequence of SEQ ID NO: 80.

39. The bispecific antibody of any one of claims 16 to 18, wherein the anti-CFHR4 HCDR1 comprises the amino acid sequence of SEQ ID NO: 58; the anti-CFHR4 HCDR2 comprises the amino acid sequence of SEQ ID NO: 70; and the anti-CFHR4 HCDR3 comprises the amino acid sequence of SEQ ID NO: 81.

40. The bispecific antibody of any one of claims 16 to 18, wherein the anti-CFHR4 HCDR1 comprises the amino acid sequence of SEQ ID NO: 59; the anti-CFHR4 HCDR2 comprises the amino acid sequence of SEQ ID NO: 71; and the anti-CFHR4 HCDR3 comprises the amino acid sequence of SEQ ID NO: 82.

41. The bispecific antibody of any one of claims 16 to 18, wherein the anti-CFHR4 HCDR1 comprises the amino acid sequence of SEQ ID NO: 60; the anti-CFHR4 HCDR2 comprises the amino acid sequence of SEQ ID NO: 72; and the anti-CFHR4 HCDR3 comprises the amino acid sequence of SEQ ID NO: 83.

42. The bispecific antibody of any one of claims 16 to 18, wherein the anti-CFHR4 HCDR1 comprises the amino acid sequence of SEQ ID NO: 61; the anti-CFHR4 HCDR2 comprises the amino acid sequence of SEQ ID NO: 73; and the anti-CFHR4 HCDR3 comprises the amino acid sequence of SEQ ID NO: 84.

43. The bispecific antibody of any one of claims 16 to 18, wherein the anti-CFHR4 HCDR1 comprises the amino acid sequence of SEQ ID NO: 62; the anti-CFHR4 HCDR2 comprises the amino acid sequence of SEQ ID NO: 74; and the anti-CFHR4 HCDR3 comprises the amino acid sequence of SEQ ID NO: 85.

44. The bispecific antibody of any one of claims 16 to 18, wherein the anti-CFHR4 HCDR1 comprises the amino acid sequence of SEQ ID NO: 63; the anti-CFHR4 HCDR2 comprises the amino acid sequence of SEQ ID NO: 75; and the anti-CFHR4 HCDR3 comprises the amino acid sequence of SEQ ID NO: 86.

45. The bispecific antibody of any one of claims 16 to 18, wherein the anti-CFHR4 HCDR1 comprises the amino acid sequence of SEQ ID NO: 64; the anti-CFHR4 HCDR2 comprises the amino acid sequence of SEQ ID NO: 76; and the anti-CFHR4 HCDR3 comprises the amino acid sequence of SEQ ID NO: 87.

46. The bispecific antibody of any one of claims 16 to 18, wherein the anti-CFHR4 HCDR1 comprises the amino acid sequence of SEQ ID NO: 89; the anti-CFHR4 HCDR2 comprises the amino acid sequence of SEQ ID NO: 103; and the anti-CFHR4 HCDR3 comprises the amino acid sequence of SEQ ID NO: 116.

47. The bispecific antibody of any one of claims 16 to 18, wherein the anti-CFHR4 HCDR1 comprises the amino acid sequence of SEQ ID NO: 90; the anti-CFHR4 HCDR2 comprises the amino acid sequence of SEQ ID NO: 104; and the anti-CFHR4 HCDR3 comprises the amino acid sequence of SEQ ID NO: 117.

48. The bispecific antibody of any one of claims 16 to 18, wherein the anti-CFHR4 HCDR1 comprises the amino acid sequence of SEQ ID NO: 91; the anti-CFHR4 HCDR2 comprises the amino acid sequence of SEQ ID NO: 105; and the anti-CFHR4 HCDR3 comprises the amino acid sequence of SEQ ID NO: 118.

49. The bispecific antibody of any one of claims 16 to 18, wherein the anti-CFHR4 HCDR1 comprises the amino acid sequence of SEQ ID NO: 92; the anti-CFHR4 HCDR2 comprises the amino acid sequence of SEQ ID NO: 106; and the anti-CFHR4 HCDR3 comprises the amino acid sequence of SEQ ID NO: 119.

50. The bispecific antibody of any one of claims 16 to 18, wherein the anti-CFHR4 HCDR1 comprises the amino acid sequence of SEQ ID NO: 93; the anti-CFHR4 HCDR2 comprises the amino acid sequence of SEQ ID NO: 107; and the anti-CFHR4 HCDR3 comprises the amino acid sequence of SEQ ID NO: 120.

51. The bispecific antibody of any one of claims 16 to 18, wherein the anti-CFHR4 HCDR1 comprises the amino acid sequence of SEQ ID NO: 94; the anti-CFHR4 HCDR2 comprises the amino acid sequence of SEQ ID NO: 108; and the anti-CFHR4 HCDR3 comprises the amino acid sequence of SEQ ID NO: 121.

52. The bispecific antibody of any one of claims 16 to 18, wherein the anti-CFHR4 HCDR1 comprises the amino acid sequence of SEQ ID NO: 95; the anti-CFHR4 HCDR2 comprises the amino acid sequence of SEQ ID NO: 109; and the anti-CFHR4 HCDR3 comprises the amino acid sequence of SEQ ID NO: 122.

53. The bispecific antibody of any one of claims 16 to 18, wherein the anti-CFHR4 HCDR1 comprises the amino acid sequence of SEQ ID NO: 96; the anti-CFHR4 HCDR2 comprises the amino acid sequence of SEQ ID NO: 110; and the anti-CFHR4 HCDR3 comprises the amino acid sequence of SEQ ID NO: 123.

54. The bispecific antibody of any one of claims 16 to 18, wherein the anti-CFHR4 HCDR1 comprises the amino acid sequence of SEQ ID NO: 97; the anti-CFHR4 HCDR2 comprises the amino acid sequence of SEQ ID NO: 111; and the anti-CFHR4 HCDR3 comprises the amino acid sequence of SEQ ID NO: 124.

55. The bispecific antibody of any one of claims 16 to 18, wherein the anti-CFHR4 HCDR1 comprises the amino acid sequence of SEQ ID NO: 98; the anti-CFHR4 HCDR2 comprises the amino acid sequence of SEQ ID NO: 112; and the anti-CFHR4 HCDR3 comprises the amino acid sequence of SEQ ID NO: 125.

56. The bispecific antibody of any one of claims 16 to 18, wherein the anti-CFHR4 HCDR1 comprises the amino acid sequence of SEQ ID NO: 99; the anti-CFHR4 HCDR2 comprises the amino acid sequence of SEQ ID NO: 113; and the HCDR3 comprises the amino acid sequence of SEQ ID NO: 126.

57. The bispecific antibody of any one of claims 16 to 18, wherein the anti-CFHR4 HCDR1 comprises the amino acid sequence of SEQ ID NO: 100; the HCDR2 comprises the amino acid sequence of SEQ ID NO: 114; and the HCDR3 comprises the amino acid sequence of SEQ ID NO: 127.

58. The bispecific antibody of any one of claims 16 to 18, wherein the anti-CFHR4 HCDR1 comprises the amino acid sequence of SEQ ID NO: 101; the anti-CFHR4 HCDR2 comprises the amino acid sequence of SEQ ID NO: 115; and the anti-CFHR4 HCDR3 comprises the amino acid sequence of SEQ ID NO: 128.

59. The bispecific antibody of any one of claims 16 to 18, wherein the anti-CFHR4 LCDR1 comprises the amino acid sequence of SEQ ID NO: 130; the anti-CFHR4 LCDR2 comprises the amino acid sequence of SEQ ID NO: 153; and the anti-CFHR4 LCDR3 comprises the amino acid sequence of SEQ ID NO: 175.

60. The bispecific antibody of any one of claims 16 to 18, wherein the anti-CFHR4 LCDR1 comprises the amino acid sequence of SEQ ID NO: 131; the anti-CFHR4 LCDR2 comprises the amino acid sequence of SEQ ID NO: 154; and the anti-CFHR4 LCDR3 comprises the amino acid sequence of SEQ ID NO: 176.

61. The bispecific antibody of any one of claims 16 to 18, wherein the anti-CFHR4 LCDR1 comprises the amino acid sequence of SEQ ID NO: 132; the anti-CFHR4 LCDR2 comprises the amino acid sequence of SEQ ID NO: 155; and the anti-CFHR4 LCDR3 comprises the amino acid sequence of SEQ ID NO: 177.

62. The bispecific antibody of any one of claims 16 to 18, wherein the anti-CFHR4 LCDR1 comprises the amino acid sequence of SEQ ID NO: 133; the anti-CFHR4 LCDR2 comprises the amino acid sequence of SEQ ID NO: 156; and the anti-CFHR4 LCDR3 comprises the amino acid sequence of SEQ ID NO: 178.

63. The bispecific antibody of any one of claims 16 to 18, wherein the anti-CFHR4 LCDR1 comprises the amino acid sequence of SEQ ID NO: 134; the anti-CFHR4 LCDR2 comprises the amino acid sequence of SEQ ID NO: 157; and the anti-CFHR4 LCDR3 comprises the amino acid sequence of SEQ ID NO: 179.

64. The bispecific antibody of any one of claims 16 to 18, wherein the anti-CFHR4 LCDR1 comprises the amino acid sequence of SEQ ID NO: 135; the anti-CFHR4 LCDR2 comprises the amino acid sequence of SEQ ID NO: 158; and the anti-CFHR4 LCDR3 comprises the amino acid sequence of SEQ ID NO: 180.

65. The bispecific antibody of any one of claims 16 to 18, wherein the anti-CFHR4 LCDR1 comprises the amino acid sequence of SEQ ID NO: 136; the anti-CFHR4 LCDR2 comprises the amino acid sequence of SEQ ID NO: 159; and the anti-CFHR4 LCDR3 comprises the amino acid sequence of SEQ ID NO: 181.

66. The bispecific antibody of any one of claims 16 to 18, wherein the anti-CFHR4 LCDR1 comprises the amino acid sequence of SEQ ID NO: 137; the anti-CFHR4 LCDR2 comprises the amino acid sequence of SEQ ID NO: 160; and the anti-CFHR4 LCDR3 comprises the amino acid sequence of SEQ ID NO: 182.

67. The bispecific antibody of any one of claims 16 to 18, wherein the anti-CFHR4 LCDR1 comprises the amino acid sequence of SEQ ID NO: 138; the anti-CFHR4 LCDR2 comprises the amino acid sequence of SEQ ID NO: 161; and the anti-CFHR4 LCDR3 comprises the amino acid sequence of SEQ ID NO: 183.

68. The bispecific antibody of any one of claims 16 to 18, wherein the anti-CFHR4 LCDR1 comprises the amino acid sequence of SEQ ID NO: 139; the anti-CFHR4 LCDR2 comprises the amino acid sequence of SEQ ID NO: 162; and the anti-CFHR4 LCDR3 comprises the amino acid sequence of SEQ ID NO: 184.

69. The bispecific antibody of any one of claims 16 to 18, wherein the anti-CFHR4 LCDR1 comprises the amino acid sequence of SEQ ID NO: 140; the anti-CFHR4 LCDR2 comprises the amino acid sequence of SEQ ID NO: 163; and the anti-CFHR4 LCDR3 comprises the amino acid sequence of SEQ ID NO: 185.

70. The bispecific antibody of any one of claims 16 to 18, wherein the anti-CFHR4 LCDR1 comprises the amino acid sequence of SEQ ID NO: 141; the anti-CFHR4 LCDR2 comprises the amino acid sequence of SEQ ID NO: 164; and the anti-CFHR4 LCDR3 comprises the amino acid sequence of SEQ ID NO: 186.

71. The bispecific antibody of any one of claims 16 to 18, wherein the anti-CFHR4 LCDR1 comprises the amino acid sequence of SEQ ID NO: 142; the anti-CFHR4 LCDR2 comprises the amino acid sequence of SEQ ID NO: 165; and the anti-CFHR4 LCDR3 comprises the amino acid sequence of SEQ ID NO: 187.

72. The bispecific antibody of any one of claims 16 to 18, wherein the anti-CFHR4 LCDR1 comprises the amino acid sequence of SEQ ID NO: 143; the anti-CFHR4 LCDR2 comprises the amino acid sequence of SEQ ID NO: 166; and the anti-CFHR4 LCDR3 comprises the amino acid sequence of SEQ ID NO: 188.

73. The bispecific antibody of any one of claims 16 to 18, wherein the anti-CFHR4 LCDR1 comprises the amino acid sequence of SEQ ID NO: 144; the anti-CFHR4 LCDR2 comprises the amino acid sequence of SEQ ID NO: 167; and the anti-CFHR4 LCDR3 comprises the amino acid sequence of SEQ ID NO: 189.

74. The bispecific antibody of any one of claims 16 to 18, wherein the anti-CFHR4 LCDR1 comprises the amino acid sequence of SEQ ID NO: 145; the anti-CFHR4 LCDR2 comprises the amino acid sequence of SEQ ID NO: 168; and the anti-CFHR4 LCDR3 comprises the amino acid sequence of SEQ ID NO: 190.

75. The bispecific antibody of any one of claims 16 to 18, wherein the anti-CFHR4 LCDR1 comprises the amino acid sequence of SEQ ID NO: 146; the anti-CFHR4 LCDR2 comprises the amino acid sequence of SEQ ID NO: 169; and the anti-CFHR4 LCDR3 comprises the amino acid sequence of SEQ ID NO: 191.

76. The bispecific antibody of any one of claims 16 to 18, wherein the anti-CFHR4 LCDR1 comprises the amino acid sequence of SEQ ID NO: 147; the anti-CFHR4 LCDR2 comprises the amino acid sequence of SEQ ID NO: 170; and the anti-CFHR4 LCDR3 comprises the amino acid sequence of SEQ ID NO: 192.

77. The bispecific antibody of any one of claims 16 to 18, wherein the anti-CFHR4 LCDR1 comprises the amino acid sequence of SEQ ID NO: 148; the anti-CFHR4 LCDR2 comprises the amino acid sequence of SEQ ID NO: 171; and the anti-CFHR4 LCDR3 comprises the amino acid sequence of SEQ ID NO: 193.

78. The bispecific antibody of any one of claims 16 to 18, wherein the anti-CFHR4 LCDR1 comprises the amino acid sequence of SEQ ID NO: 149; the anti-CFHR4 LCDR2 comprises the amino acid sequence of SEQ ID NO: 172; and the anti-CFHR4 LCDR3 comprises the amino acid sequence of SEQ ID NO: 194.

79. The bispecific antibody of any one of claims 16 to 18, wherein the anti-CFHR4 LCDR1 comprises the amino acid sequence of SEQ ID NO: 150; the anti-CFHR4 LCDR2 comprises the amino acid sequence of SEQ ID NO: 173; and the anti-CFHR4 LCDR3 comprises the amino acid sequence of SEQ ID NO: 195.

80. The bispecific antibody of any one of claims 16 to 18, wherein the anti-CFHR4 LCDR1 comprises the amino acid sequence of SEQ ID NO: 151; the anti-CFHR4 LCDR2 comprises the amino acid sequence of SEQ ID NO: 174; and the anti-CFHR4 LCDR3 comprises the amino acid sequence of SEQ ID NO: 196.

81. The bispecific antibody of any one of claims 16 to 18, wherein the anti-CFHR4 LCDR1 comprises the amino acid sequence of SEQ ID NO: 198; the anti-CFHR4 LCDR2 comprises the amino acid sequence of SEQ ID NO: 201; and the anti-CFHR4 LCDR3 comprises the amino acid sequence of SEQ ID NO: 204.

82. The bispecific antibody of any one of claims 16 to 18, wherein the anti-CFHR4 LCDR1 comprises the amino acid sequence of SEQ ID NO: 199; the anti-CFHR4 LCDR2 comprises the amino acid sequence of SEQ ID NO: 202; and the anti-CFHR4 LCDR3 comprises the amino acid sequence of SEQ ID NO: 205.

83. The bispecific antibody of any one of claims 16 to 18, wherein the anti-CFHR4 LCDR1 comprises the amino acid sequence of SEQ ID NO: 207; the anti-CFHR4 LCDR2 comprises the amino acid sequence of SEQ ID NO: 217; and the anti-CFHR4 LCDR3 comprises the amino acid sequence of SEQ ID NO: 227.

84. The bispecific antibody of any one of claims 16 to 18, wherein the anti-CFHR4 LCDR1 comprises the amino acid sequence of SEQ ID NO: 208; the anti-CFHR4 LCDR2 comprises the amino acid sequence of SEQ ID NO: 218; and the anti-CFHR4 LCDR3 comprises the amino acid sequence of SEQ ID NO: 228.

85. The bispecific antibody of any one of claims 16 to 18, wherein the anti-CFHR4 LCDR1 comprises the amino acid sequence of SEQ ID NO: 209; the anti-CFHR4 LCDR2 comprises the amino acid sequence of SEQ ID NO: 219; and the anti-CFHR4 LCDR3 comprises the amino acid sequence of SEQ ID NO: 229.

86. The bispecific antibody of any one of claims 16 to 18, wherein the anti-CFHR4 LCDR1 comprises the amino acid sequence of SEQ ID NO: 210; the anti-CFHR4 LCDR2 comprises the amino acid sequence of SEQ ID NO: 220; and the anti-CFHR4 LCDR3 comprises the amino acid sequence of SEQ ID NO: 230.

87. The bispecific antibody of any one of claims 16 to 18, wherein the anti-CFHR4 LCDR1 comprises the amino acid sequence of SEQ ID NO: 211; the anti-CFHR4 LCDR2 comprises the amino acid sequence of SEQ ID NO: 221; and the anti-CFHR4 LCDR3 comprises the amino acid sequence of SEQ ID NO: 231.

88. The bispecific antibody of any one of claims 16 to 18, wherein the anti-CFHR4 LCDR1 comprises the amino acid sequence of SEQ ID NO: 212; the anti-CFHR4 LCDR2 comprises the amino acid sequence of SEQ ID NO: 222; and the anti-CFHR4 LCDR3 comprises the amino acid sequence of SEQ ID NO: 232.

89. The bispecific antibody of any one of claims 16 to 18, wherein the anti-CFHR4 LCDR1 comprises the amino acid sequence of SEQ ID NO: 213; the anti-CFHR4 LCDR2 comprises the amino acid sequence of SEQ ID NO: 223; and the anti-CFHR4 LCDR3 comprises the amino acid sequence of SEQ ID NO: 233.

90. The bispecific antibody of any one of claims 16 to 18, wherein the anti-CFHR4 LCDR1 comprises the amino acid sequence of SEQ ID NO: 214; the anti-CFHR4 LCDR2 comprises the amino acid sequence of SEQ ID NO: 224; and the anti-CFHR4 LCDR3 comprises the amino acid sequence of SEQ ID NO: 234.

91. The bispecific antibody of any one of claims 16 to 18, wherein the anti-CFHR4 LCDR1 comprises the amino acid sequence of SEQ ID NO: 215; the anti-CFHR4 LCDR2 comprises the amino acid sequence of SEQ ID NO: 225; and the anti-CFHR4 LCDR3 comprises the amino acid sequence of SEQ ID NO: 235.

92. The bispecific antibody of any one of claims 16 to 18, wherein the anti-CFHR4 LCDR1 comprises the amino acid sequence of SEQ ID NO: 237; the anti-CFHR4 LCDR2 comprises the amino acid sequence of SEQ ID NO: 245; and the anti-CFHR4 LCDR3 comprises the amino acid sequence of SEQ ID NO: 253.

93. The bispecific antibody of any one of claims 16 to 18, wherein the anti-CFHR4 LCDR1 comprises the amino acid sequence of SEQ ID NO: 238; the anti-CFHR4 LCDR2 comprises the amino acid sequence of SEQ ID NO: 246; and the anti-CFHR4 LCDR3 comprises the amino acid sequence of SEQ ID NO: 254.

94. The bispecific antibody of any one of claims 16 to 18, wherein the anti-CFHR4 LCDR1 comprises the amino acid sequence of SEQ ID NO: 239; the anti-CFHR4 LCDR2 comprises the amino acid sequence of SEQ ID NO: 247; and the anti-CFHR4 LCDR3 comprises the amino acid sequence of SEQ ID NO: 255.

95. The bispecific antibody of any one of claims 16 to 18, wherein the anti-CFHR4 LCDR1 comprises the amino acid sequence of SEQ ID NO: 240; the anti-CFHR4 LCDR2 comprises the amino acid sequence of SEQ ID NO: 248; and the anti-CFHR4 LCDR3 comprises the amino acid sequence of SEQ ID NO: 256.

96. The bispecific antibody of any one of claims 16 to 18, wherein the anti-CFHR4 LCDR1 comprises the amino acid sequence of SEQ ID NO: 241; the anti-CFHR4 LCDR2 comprises the amino acid sequence of SEQ ID NO: 249; and the anti-CFHR4 LCDR3 comprises the amino acid sequence of SEQ ID NO: 257.

97. The bispecific antibody of any one of claims 16 to 18, wherein the anti-CFHR4 LCDR1 comprises the amino acid sequence of SEQ ID NO: 242; the anti-CFHR4 LCDR2 comprises the amino acid sequence of SEQ ID NO: 250; and the anti-CFHR4 LCDR3 comprises the amino acid sequence of SEQ ID NO: 258.

98. The bispecific antibody of any one of claims 16 to 18, wherein the anti-CFHR4 LCDR1 comprises the amino acid sequence of SEQ ID NO: 243; the anti-CFHR4 LCDR2 comprises the amino acid sequence of SEQ ID NO: 251; and the anti-CFHR4 LCDR3 comprises the amino acid sequence of SEQ ID NO: 259.

99. The bispecific antibody of any one of claims 16 to 18, wherein the anti-C3 HCDR1 comprises the amino acid sequence of SEQ ID NO: 583; the anti-C3 HCDR2 comprises the amino acid sequence of SEQ ID NO: 602; and the anti-C3 HCDR3 comprises the amino acid sequence of SEQ ID NO: 620.

100. The bispecific antibody of any one of claims 16 to 18, wherein the anti-C3 HCDR1 comprises the amino acid sequence of SEQ ID NO: 584; the anti-C3 HCDR2 comprises the amino acid sequence of SEQ ID NO: 603; and the anti-C3 HCDR3 comprises the amino acid sequence of SEQ ID NO: 621.

101. The bispecific antibody of any one of claims 16 to 18, wherein the anti-C3 HCDR1 comprises the amino acid sequence of SEQ ID NO: 585; the anti-C3 HCDR2 comprises the amino acid sequence of SEQ ID NO: 604; and the anti-C3 HCDR3 comprises the amino acid sequence of SEQ ID NO: 622.

102. The bispecific antibody of any one of claims 16 to 18, wherein the anti-C3 HCDR1 comprises the amino acid sequence of SEQ ID NO: 586; the anti-C3 HCDR2 comprises the amino acid sequence of SEQ ID NO: 605; and the anti-C3 HCDR3 comprises the amino acid sequence of SEQ ID NO: 623.

103. The bispecific antibody of any one of claims 16 to 18, wherein the anti-C3 HCDR1 comprises the amino acid sequence of SEQ ID NO: 587; the anti-C3 HCDR2 comprises the amino acid sequence of SEQ ID NO: 606; and the anti-C3 HCDR3 comprises the amino acid sequence of SEQ ID NO: 624.

104. The bispecific antibody of any one of claims 16 to 18, wherein the anti-C3 HCDR1 comprises the amino acid sequence of SEQ ID NO: 588; the anti-C3 HCDR2 comprises the amino acid sequence of SEQ ID NO: 607; and the anti-C3 HCDR3 comprises the amino acid sequence of SEQ ID NO: 625.

105. The bispecific antibody of any one of claims 16 to 18, wherein the anti-C3 HCDR1 comprises the amino acid sequence of SEQ ID NO: 589; the anti-C3 HCDR2 comprises the amino acid sequence of SEQ ID NO: 608; and the anti-C3 HCDR3 comprises the amino acid sequence of SEQ ID NO: 626.

106. The bispecific antibody of any one of claims 16 to 18, wherein the anti-C3 HCDR1 comprises the amino acid sequence of SEQ ID NO: 590; the anti-C3 HCDR2 comprises the amino acid sequence of SEQ ID NO: 609; and the anti-C3 HCDR3 comprises the amino acid sequence of SEQ ID NO: 627.

107. The bispecific antibody of any one of claims 16 to 18, wherein the anti-C3 HCDR1 comprises the amino acid sequence of SEQ ID NO: 591; the anti-C3 HCDR2 comprises the amino acid sequence of SEQ ID NO: 610; and the anti-C3 HCDR3 comprises the amino acid sequence of SEQ ID NO: 628.

108. The bispecific antibody of any one of claims 16 to 18, wherein the anti-C3 HCDR1 comprises the amino acid sequence of SEQ ID NO: 592; the anti-C3 HCDR2 comprises the amino acid sequence of SEQ ID NO: 611; and the anti-C3 HCDR3 comprises the amino acid sequence of SEQ ID NO: 629.

109. The bispecific antibody of any one of claims 16 to 18, wherein the anti-C3 HCDR1 comprises the amino acid sequence of SEQ ID NO: 593; the anti-C3 HCDR2 comprises the amino acid sequence of SEQ ID NO: 612; and the anti-C3 HCDR3 comprises the amino acid sequence of SEQ ID NO: 630.

110. The bispecific antibody of any one of claims 16 to 18, wherein the anti-C3 HCDR1 comprises the amino acid sequence of SEQ ID NO: 594; the anti-C3 HCDR2 comprises the amino acid sequence of SEQ ID NO: 613; and the anti-C3 HCDR3 comprises the amino acid sequence of SEQ ID NO: 631.

111. The bispecific antibody of any one of claims 16 to 18, wherein the anti-C3 HCDR1 comprises the amino acid sequence of SEQ ID NO: 595; the anti-C3 HCDR2 comprises the amino acid sequence of SEQ ID NO: 614; and the anti-C3 HCDR3 comprises the amino acid sequence of SEQ ID NO: 632.

112. The bispecific antibody of any one of claims 16 to 18, wherein the anti-C3 HCDR1 comprises the amino acid sequence of SEQ ID NO: 596; the anti-C3 HCDR2 comprises the amino acid sequence of SEQ ID NO: 615; and the anti-C3 HCDR3 comprises the amino acid sequence of SEQ ID NO: 633.

113. The bispecific antibody of any one of claims 16 to 18, wherein the anti-C3 HCDR1 comprises the amino acid sequence of SEQ ID NO: 597; the anti-C3 HCDR2 comprises the amino acid sequence of SEQ ID NO: 616; and the anti-C3 HCDR3 comprises the amino acid sequence of SEQ ID NO: 634.

114. The bispecific antibody of any one of claims 16 to 18, wherein the anti-C3 HCDR1 comprises the amino acid sequence of SEQ ID NO: 598; the anti-C3 HCDR2 comprises the amino acid sequence of SEQ ID NO: 617; and the anti-C3 HCDR3 comprises the amino acid sequence of SEQ ID NO: 635.

115. The bispecific antibody of any one of claims 16 to 18, wherein the anti-C3 HCDR1 comprises the amino acid sequence of SEQ ID NO: 599; the anti-C3 HCDR2 comprises the amino acid sequence of SEQ ID NO: 618; and the anti-C3 HCDR3 comprises the amino acid sequence of SEQ ID NO: 636.

116. The bispecific antibody of any one of claims 16 to 18, wherein the anti-C3 HCDR1 comprises the amino acid sequence of SEQ ID NO: 600; the anti-C3 HCDR2 comprises the amino acid sequence of SEQ ID NO: 619; and the anti-C3 HCDR3 comprises the amino acid sequence of SEQ ID NO: 637.

117. The bispecific antibody of any one of claims 16 to 18, wherein the anti-C3 HCDR1 comprises the amino acid sequence of SEQ ID NO: 639; the anti-C3 HCDR2 comprises the amino acid sequence of SEQ ID NO: 652; and the anti-C3 HCDR3 comprises the amino acid sequence of SEQ ID NO: 664.

118. The bispecific antibody of any one of claims 16 to 18, wherein the anti-C3 HCDR1 comprises the amino acid sequence of SEQ ID NO: 640; the anti-C3 HCDR2 comprises the amino acid sequence of SEQ ID NO: 653; and the anti-C3 HCDR3 comprises the amino acid sequence of SEQ ID NO: 665.

119. The bispecific antibody of any one of claims 16 to 18, wherein the anti-C3 HCDR1 comprises the amino acid sequence of SEQ ID NO: 641; the anti-C3 HCDR2 comprises the amino acid sequence of SEQ ID NO: 654; and the anti-C3 HCDR3 comprises the amino acid sequence of SEQ ID NO: 666.

120. The bispecific antibody of any one of claims 16 to 18, wherein the anti-C3 HCDR1 comprises the amino acid sequence of SEQ ID NO: 642; the anti-C3 HCDR2 comprises the amino acid sequence of SEQ ID NO: 655; and the anti-C3 HCDR3 comprises the amino acid sequence of SEQ ID NO: 667.

121. The bispecific antibody of any one of claims 16 to 18, wherein the anti-C3 HCDR1 comprises the amino acid sequence of SEQ ID NO: 643; the anti-C3 HCDR2 comprises the amino acid sequence of SEQ ID NO: 656; and the anti-C3 HCDR3 comprises the amino acid sequence of SEQ ID NO: 668.

122. The bispecific antibody of any one of claims 16 to 18, wherein the anti-C3 HCDR1 comprises the amino acid sequence of SEQ ID NO: 644; the anti-C3 HCDR2 comprises the amino acid sequence of SEQ ID NO: 657; and the anti-C3 HCDR3 comprises the amino acid sequence of SEQ ID NO: 669.

123. The bispecific antibody of any one of claims 16 to 18, wherein the anti-C3 HCDR1 comprises the amino acid sequence of SEQ ID NO: 645; the anti-C3 HCDR2 comprises the amino acid sequence of SEQ ID NO: 658; and the anti-C3 HCDR3 comprises the amino acid sequence of SEQ ID NO: 670.

124. The bispecific antibody of any one of claims 16 to 18, wherein the anti-C3 HCDR1 comprises the amino acid sequence of SEQ ID NO: 646; the anti-C3 HCDR2 comprises the amino acid sequence of SEQ ID NO: 659; and the anti-C3 HCDR3 comprises the amino acid sequence of SEQ ID NO: 671.

125. The bispecific antibody of any one of claims 16 to 18, wherein the anti-C3 HCDR1 comprises the amino acid sequence of SEQ ID NO: 647; the anti-C3 HCDR2 comprises the amino acid sequence of SEQ ID NO: 660; and the anti-C3 HCDR3 comprises the amino acid sequence of SEQ ID NO: 672.

126. The bispecific antibody of any one of claims 16 to 18, wherein the anti-C3 HCDR1 comprises the amino acid sequence of SEQ ID NO: 648; the anti-C3 HCDR2 comprises the amino acid sequence of SEQ ID NO: 661; and the anti-C3 HCDR3 comprises the amino acid sequence of SEQ ID NO: 673.

127. The bispecific antibody of any one of claims 16 to 18, wherein the anti-C3 HCDR1 comprises the amino acid sequence of SEQ ID NO: 649; the anti-C3 HCDR2 comprises the amino acid sequence of SEQ ID NO: 662; and the anti-C3 HCDR3 comprises the amino acid sequence of SEQ ID NO: 674.

128. The bispecific antibody of any one of claims 16 to 18, wherein the anti-C3 HCDR1 comprises the amino acid sequence of SEQ ID NO: 650; the anti-C3 HCDR2 comprises the amino acid sequence of SEQ ID NO: 663; and the anti-C3 HCDR3 comprises the amino acid sequence of SEQ ID NO: 675.

129. The bispecific antibody of any one of claims 16 to 18, wherein the anti-C3 HCDR1 comprises the amino acid sequence of SEQ ID NO: 677; the anti-C3 HCDR2 comprises the amino acid sequence of SEQ ID NO: 706; and the anti-C3 HCDR3 comprises the amino acid sequence of SEQ ID NO: 734.

130. The bispecific antibody of any one of claims 16 to 18, wherein the anti-C3 HCDR1 comprises the amino acid sequence of SEQ ID NO: 678; the anti-C3 HCDR2 comprises the amino acid sequence of SEQ ID NO: 707; and the anti-C3 HCDR3 comprises the amino acid sequence of SEQ ID NO: 735.

131. The bispecific antibody of any one of claims 16 to 18, wherein the anti-C3 HCDR1 comprises the amino acid sequence of SEQ ID NO: 679; the anti-C3 HCDR2 comprises the amino acid sequence of SEQ ID NO: 708; and the anti-C3 HCDR3 comprises the amino acid sequence of SEQ ID NO: 736.

132. The bispecific antibody of any one of claims 16 to 18, wherein the anti-C3 HCDR1 comprises the amino acid sequence of SEQ ID NO: 680; the anti-C3 HCDR2 comprises the amino acid sequence of SEQ ID NO: 709; and the anti-C3 HCDR3 comprises the amino acid sequence of SEQ ID NO: 737.

133. The bispecific antibody of any one of claims 16 to 18, wherein the anti-C3 HCDR1 comprises the amino acid sequence of SEQ ID NO: 681; the anti-C3 HCDR2 comprises the amino acid sequence of SEQ ID NO: 710; and the anti-C3 HCDR3 comprises the amino acid sequence of SEQ ID NO: 738.

134. The bispecific antibody of any one of claims 16 to 18, wherein the anti-C3 HCDR1 comprises the amino acid sequence of SEQ ID NO: 682; the anti-C3 HCDR2 comprises the amino acid sequence of SEQ ID NO: 711; and the anti-C3 HCDR3 comprises the amino acid sequence of SEQ ID NO: 739.

135. The bispecific antibody of any one of claims 16 to 18, wherein the anti-C3 HCDR1 comprises the amino acid sequence of SEQ ID NO: 683; the anti-C3 HCDR2 comprises the amino acid sequence of SEQ ID NO: 712; and the anti-C3 HCDR3 comprises the amino acid sequence of SEQ ID NO: 740.

136. The bispecific antibody of any one of claims 16 to 18, wherein the anti-C3 HCDR1 comprises the amino acid sequence of SEQ ID NO: 684; the anti-C3 HCDR2 comprises the amino acid sequence of SEQ ID NO: 713; and the anti-C3 HCDR3 comprises the amino acid sequence of SEQ ID NO: 741.

137. The bispecific antibody of any one of claims 16 to 18, wherein the anti-C3 HCDR1 comprises the amino acid sequence of SEQ ID NO: 685; the anti-C3 HCDR2 comprises the amino acid sequence of SEQ ID NO: 714; and the anti-C3 HCDR3 comprises the amino acid sequence of SEQ ID NO: 742.

138. The bispecific antibody of any one of claims 16 to 18, wherein the anti-C3 HCDR1 comprises the amino acid sequence of SEQ ID NO: 686; the anti-C3 HCDR2 comprises the amino acid sequence of SEQ ID NO: 715; and the anti-C3 HCDR3 comprises the amino acid sequence of SEQ ID NO: 743.

139. The bispecific antibody of any one of claims 16 to 18, wherein the anti-C3 HCDR1 comprises the amino acid sequence of SEQ ID NO: 687; the anti-C3 HCDR2 comprises the amino acid sequence of SEQ ID NO: 716; and the anti-C3 HCDR3 comprises the amino acid sequence of SEQ ID NO: 744.

140. The bispecific antibody of any one of claims 16 to 18, wherein the anti-C3 HCDR1 comprises the amino acid sequence of SEQ ID NO: 688; the anti-C3 HCDR2 comprises the amino acid sequence of SEQ ID NO: 717; and the anti-C3 HCDR3 comprises the amino acid sequence of SEQ ID NO: 745.

141. The bispecific antibody of any one of claims 16 to 18, wherein the anti-C3 HCDR1 comprises the amino acid sequence of SEQ ID NO: 689; the anti-C3 HCDR2 comprises the amino acid sequence of SEQ ID NO: 718; and the anti-C3 HCDR3 comprises the amino acid sequence of SEQ ID NO: 746.

142. The bispecific antibody of any one of claims 16 to 18, wherein the anti-C3 HCDR1 comprises the amino acid sequence of SEQ ID NO: 690; the anti-C3 HCDR2 comprises the amino acid sequence of SEQ ID NO: 719; and the anti-C3 HCDR3 comprises the amino acid sequence of SEQ ID NO: 747.

143. The bispecific antibody of any one of claims 16 to 18, wherein the anti-C3 HCDR1 comprises the amino acid sequence of SEQ ID NO: 691; the anti-C3 HCDR2 comprises the amino acid sequence of SEQ ID NO: 720; and the anti-C3 HCDR3 comprises the amino acid sequence of SEQ ID NO: 748.

144. The bispecific antibody of any one of claims 16 to 18, wherein the anti-C3 HCDR1 comprises the amino acid sequence of SEQ ID NO: 692; the anti-C3 HCDR2 comprises the amino acid sequence of SEQ ID NO: 721; and the anti-C3 HCDR3 comprises the amino acid sequence of SEQ ID NO: 749.

145. The bispecific antibody of any one of claims 16 to 18, wherein the anti-C3 HCDR1 comprises the amino acid sequence of SEQ ID NO: 693; the anti-C3 HCDR2 comprises the amino acid sequence of SEQ ID NO: 722; and the anti-C3 HCDR3 comprises the amino acid sequence of SEQ ID NO: 750.

146. The bispecific antibody of any one of claims 16 to 18, wherein the anti-C3 HCDR1 comprises the amino acid sequence of SEQ ID NO: 694; the anti-C3 HCDR2 comprises the amino acid sequence of SEQ ID NO: 723; and the anti-C3 HCDR3 comprises the amino acid sequence of SEQ ID NO: 751.

147. The bispecific antibody of any one of claims 16 to 18, wherein the anti-C3 HCDR1 comprises the amino acid sequence of SEQ ID NO: 695; the anti-C3 HCDR2 comprises the amino acid sequence of SEQ ID NO: 724; and the anti-C3 HCDR3 comprises the amino acid sequence of SEQ ID NO: 752.

148. The bispecific antibody of any one of claims 16 to 18, wherein the anti-C3 HCDR1 comprises the amino acid sequence of SEQ ID NO: 696; the anti-C3 HCDR2 comprises the amino acid sequence of SEQ ID NO: 725; and the anti-C3 HCDR3 comprises the amino acid sequence of SEQ ID NO: 753.

149. The bispecific antibody of any one of claims 16 to 18, wherein the anti-C3 HCDR1 comprises the amino acid sequence of SEQ ID NO: 697; the anti-C3 HCDR2 comprises the amino acid sequence of SEQ ID NO: 726; and the anti-C3 HCDR3 comprises the amino acid sequence of SEQ ID NO: 754.

150. The bispecific antibody of any one of claims 16 to 18, wherein the anti-C3 HCDR1 comprises the amino acid sequence of SEQ ID NO: 698; the anti-C3 HCDR2 comprises the amino acid sequence of SEQ ID NO: 727; and the anti-C3 HCDR3 comprises the amino acid sequence of SEQ ID NO: 755.

151. The bispecific antibody of any one of claims 16 to 18, wherein the anti-C3 HCDR1 comprises the amino acid sequence of SEQ ID NO: 699; the anti-C3 HCDR2 comprises the amino acid sequence of SEQ ID NO: 728; and the anti-C3 HCDR3 comprises the amino acid sequence of SEQ ID NO: 756.

152. The bispecific antibody of any one of claims 16 to 18, wherein the anti-C3 HCDR1 comprises the amino acid sequence of SEQ ID NO: 700; the anti-C3 HCDR2 comprises the amino acid sequence of SEQ ID NO: 729; and the anti-C3 HCDR3 comprises the amino acid sequence of SEQ ID NO: 757.

153. The bispecific antibody of any one of claims 16 to 18, wherein the anti-C3 HCDR1 comprises the amino acid sequence of SEQ ID NO: 701; the anti-C3 HCDR2 comprises the amino acid sequence of SEQ ID NO: 730; and the anti-C3 HCDR3 comprises the amino acid sequence of SEQ ID NO: 758.

154. The bispecific antibody of any one of claims 16 to 18, wherein the anti-C3 HCDR1 comprises the amino acid sequence of SEQ ID NO: 702; the anti-C3 HCDR2 comprises the amino acid sequence of SEQ ID NO: 731; and the anti-C3 HCDR3 comprises the amino acid sequence of SEQ ID NO: 759.

155. The bispecific antibody of any one of claims 16 to 18, wherein the anti-C3 HCDR1 comprises the amino acid sequence of SEQ ID NO: 703; the anti-C3 HCDR2 comprises the amino acid sequence of SEQ ID NO: 732; and the anti-C3 HCDR3 comprises the amino acid sequence of SEQ ID NO: 760.

156. The bispecific antibody of any one of claims 16 to 18, wherein the anti-C3 HCDR1 comprises the amino acid sequence of SEQ ID NO: 704; the anti-C3 HCDR2 comprises the amino acid sequence of SEQ ID NO: 733; and the anti-C3 HCDR3 comprises the amino acid sequence of SEQ ID NO: 761.

157. The bispecific antibody of any one of claims 16 to 18, wherein the anti-C3 HCDR1 comprises the amino acid sequence of SEQ ID NO: 763; the anti-C3 HCDR2 comprises the amino acid sequence of SEQ ID NO: 771; and the anti-C3 HCDR3 comprises the amino acid sequence of SEQ ID NO: 778.

158. The bispecific antibody of any one of claims 16 to 18, wherein the anti-C3 HCDR1 comprises the amino acid sequence of SEQ ID NO: 764; the anti-C3 HCDR2 comprises the amino acid sequence of SEQ ID NO: 772; and the anti-C3 HCDR3 comprises the amino acid sequence of SEQ ID NO: 779.

159. The bispecific antibody of any one of claims 16 to 18, wherein the anti-C3 HCDR1 comprises the amino acid sequence of SEQ ID NO: 765; the anti-C3 HCDR2 comprises the amino acid sequence of SEQ ID NO: 773; and the anti-C3 HCDR3 comprises the amino acid sequence of SEQ ID NO: 780.

160. The bispecific antibody of any one of claims 16 to 18, wherein the anti-C3 HCDR1 comprises the amino acid sequence of SEQ ID NO: 766; the anti-C3 HCDR2 comprises the amino acid sequence of SEQ ID NO: 774; and the anti-C3 HCDR3 comprises the amino acid sequence of SEQ ID NO: 781.

161. The bispecific antibody of any one of claims 16 to 18, wherein the anti-C3 HCDR1 comprises the amino acid sequence of SEQ ID NO: 767; the anti-C3 HCDR2 comprises the amino acid sequence of SEQ ID NO: 775; and the anti-C3 HCDR3 comprises the amino acid sequence of SEQ ID NO: 782.

162. The bispecific antibody of any one of claims 16 to 18, wherein the anti-C3 HCDR1 comprises the amino acid sequence of SEQ ID NO: 768; the anti-C3 HCDR2 comprises the amino acid sequence of SEQ ID NO: 776; and the anti-C3 HCDR3 comprises the amino acid sequence of SEQ ID NO: 783.

163. The bispecific antibody of any one of claims 16 to 18, wherein the anti-C3 HCDR1 comprises the amino acid sequence of SEQ ID NO: 769; the anti-C3 HCDR2 comprises the amino acid sequence of SEQ ID NO: 777; and the anti-C3 HCDR3 comprises the amino acid sequence of SEQ ID NO: 784.

164. The bispecific antibody of any one of claims 16 to 18, wherein the anti-C3 HCDR1 comprises the amino acid sequence of SEQ ID NO: 786; the anti-C3 HCDR2 comprises the amino acid sequence of SEQ ID NO: 794; and the anti-C3 HCDR3 comprises the amino acid sequence of SEQ ID NO: 801.

165. The bispecific antibody of any one of claims 16 to 18, wherein the anti-C3 HCDR1 comprises the amino acid sequence of SEQ ID NO: 787; the anti-C3 HCDR2 comprises the amino acid sequence of SEQ ID NO: 795; and the anti-C3 HCDR3 comprises the amino acid sequence of SEQ ID NO: 802.

166. The bispecific antibody of any one of claims 16 to 18, wherein the anti-C3 HCDR1 comprises the amino acid sequence of SEQ ID NO: 788; the anti-C3 HCDR2 comprises the amino acid sequence of SEQ ID NO: 796; and the anti-C3 HCDR3 comprises the amino acid sequence of SEQ ID NO: 803.

167. The bispecific antibody of any one of claims 16 to 18, wherein the anti-C3 HCDR1 comprises the amino acid sequence of SEQ ID NO: 789; the anti-C3 HCDR2 comprises the amino acid sequence of SEQ ID NO: 797; and the anti-C3 HCDR3 comprises the amino acid sequence of SEQ ID NO: 804.

168. The bispecific antibody of any one of claims 16 to 18, wherein the anti-C3 HCDR1 comprises the amino acid sequence of SEQ ID NO: 790; the anti-C3 HCDR2 comprises the amino acid sequence of SEQ ID NO: 798; and the anti-C3 HCDR3 comprises the amino acid sequence of SEQ ID NO: 805.

169. The bispecific antibody of any one of claims 16 to 18, wherein the anti-C3 HCDR1 comprises the amino acid sequence of SEQ ID NO: 791; the anti-C3 HCDR2 comprises the amino acid sequence of SEQ ID NO: 799; and the anti-C3 HCDR3 comprises the amino acid sequence of SEQ ID NO: 806.

170. The bispecific antibody of any one of claims 16 to 18, wherein the anti-C3 HCDR1 comprises the amino acid sequence of SEQ ID NO: 792; the anti-C3 HCDR2 comprises the amino acid sequence of SEQ ID NO: 800; and the anti-C3 HCDR3 comprises the amino acid sequence of SEQ ID NO: 807.

171. The bispecific antibody of any one of claims 16 to 18, wherein the anti-C3 HCDR1 comprises the amino acid sequence of SEQ ID NO: 809; the anti-C3 HCDR2 comprises the amino acid sequence of SEQ ID NO: 812; and the anti-C3 HCDR3 comprises the amino acid sequence of SEQ ID NO: 815.

172. The bispecific antibody of any one of claims 16 to 18, wherein the anti-C3 HCDR1 comprises the amino acid sequence of SEQ ID NO: 810; the anti-C3 HCDR2 comprises the amino acid sequence of SEQ ID NO: 813; and the anti-C3 HCDR3 comprises the amino acid sequence of SEQ ID NO: 816.

173. The bispecific antibody of any one of claims 16 to 18, wherein the anti-C3 LCDR1 comprises the amino acid sequence of SEQ ID NO: 818; the anti-C3 LCDR2 comprises the amino acid sequence of SEQ ID NO: 850; and the anti-C3 LCDR3 comprises the amino acid sequence of SEQ ID NO: 881.

174. The bispecific antibody of any one of claims 16 to 18, wherein the anti-C3 LCDR1 comprises the amino acid sequence of SEQ ID NO: 819; the anti-C3 LCDR2 comprises the amino acid sequence of SEQ ID NO: 851; and the anti-C3 LCDR3 comprises the amino acid sequence of SEQ ID NO: 882.

175. The bispecific antibody of any one of claims 16 to 18, wherein the anti-C3 LCDR1 comprises the amino acid sequence of SEQ ID NO: 820; the anti-C3 LCDR2 comprises the amino acid sequence of SEQ ID NO: 852; and the anti-C3 LCDR3 comprises the amino acid sequence of SEQ ID NO: 883.

176. The bispecific antibody of any one of claims 16 to 18, wherein the anti-C3 LCDR1 comprises the amino acid sequence of SEQ ID NO: 821; the anti-C3 LCDR2 comprises the amino acid sequence of SEQ ID NO: 853; and the anti-C3 LCDR3 comprises the amino acid sequence of SEQ ID NO: 884.

177. The bispecific antibody of any one of claims 16 to 18, wherein the anti-C3 LCDR1 comprises the amino acid sequence of SEQ ID NO: 822; the anti-C3 LCDR2 comprises the amino acid sequence of SEQ ID NO: 854; and the anti-C3 LCDR3 comprises the amino acid sequence of SEQ ID NO: 885.

178. The bispecific antibody of any one of claims 16 to 18, wherein the anti-C3 LCDR1 comprises the amino acid sequence of SEQ ID NO: 823; the anti-C3 LCDR2 comprises the amino acid sequence of SEQ ID NO: 855; and the anti-C3 LCDR3 comprises the amino acid sequence of SEQ ID NO: 886.

179. The bispecific antibody of any one of claims 16 to 18, wherein the anti-C3 LCDR1 comprises the amino acid sequence of SEQ ID NO: 824; the anti-C3 LCDR2 comprises the amino acid sequence of SEQ ID NO: 856; and the anti-C3 LCDR3 comprises the amino acid sequence of SEQ ID NO: 887.

180. The bispecific antibody of any one of claims 16 to 18, wherein the anti-C3 LCDR1 comprises the amino acid sequence of SEQ ID NO: 825; the anti-C3 LCDR2 comprises the amino acid sequence of SEQ ID NO: 857; and the anti-C3 LCDR3 comprises the amino acid sequence of SEQ ID NO: 888.

181. The bispecific antibody of any one of claims 16 to 18, wherein the anti-C3 LCDR1 comprises the amino acid sequence of SEQ ID NO: 826; the anti-C3 LCDR2 comprises the amino acid sequence of SEQ ID NO: 858; and the anti-C3 LCDR3 comprises the amino acid sequence of SEQ ID NO: 889.

182. The bispecific antibody of any one of claims 16 to 18, wherein the anti-C3 LCDR1 comprises the amino acid sequence of SEQ ID NO: 827; the anti-C3 LCDR2 comprises the amino acid sequence of SEQ ID NO: 859; and the anti-C3 LCDR3 comprises the amino acid sequence of SEQ ID NO: 890.

183. The bispecific antibody of any one of claims 16 to 18, wherein the anti-C3 LCDR1 comprises the amino acid sequence of SEQ ID NO: 828; the anti-C3 LCDR2 comprises the amino acid sequence of SEQ ID NO: 860; and the anti-C3 LCDR3 comprises the amino acid sequence of SEQ ID NO: 891.

184. The bispecific antibody of any one of claims 16 to 18, wherein the anti-C3 LCDR1 comprises the amino acid sequence of SEQ ID NO: 829; the anti-C3 LCDR2 comprises the amino acid sequence of SEQ ID NO: 861; and the anti-C3 LCDR3 comprises the amino acid sequence of SEQ ID NO: 892.

185. The bispecific antibody of any one of claims 16 to 18, wherein the anti-C3 LCDR1 comprises the amino acid sequence of SEQ ID NO: 830; the anti-C3 LCDR2 comprises the amino acid sequence of SEQ ID NO: 862; and the anti-C3 LCDR3 comprises the amino acid sequence of SEQ ID NO: 893.

186. The bispecific antibody of any one of claims 16 to 18, wherein the anti-C3 LCDR1 comprises the amino acid sequence of SEQ ID NO: 831; the anti-C3 LCDR2 comprises the amino acid sequence of SEQ ID NO: 863; and the anti-C3 LCDR3 comprises the amino acid sequence of SEQ ID NO: 894.

187. The bispecific antibody of any one of claims 16 to 18, wherein the anti-C3 LCDR1 comprises the amino acid sequence of SEQ ID NO: 832; the anti-C3 LCDR2 comprises the amino acid sequence of SEQ ID NO: 864; and the anti-C3 LCDR3 comprises the amino acid sequence of SEQ ID NO: 895.

188. The bispecific antibody of any one of claims 16 to 18, wherein the anti-C3 LCDR1 comprises the amino acid sequence of SEQ ID NO: 833; the anti-C3 LCDR2 comprises the amino acid sequence of SEQ ID NO: 865; and the anti-C3 LCDR3 comprises the amino acid sequence of SEQ ID NO: 896.

189. The bispecific antibody of any one of claims 16 to 18, wherein the anti-C3 LCDR1 comprises the amino acid sequence of SEQ ID NO: 834; the anti-C3 LCDR2 comprises the amino acid sequence of SEQ ID NO: 866; and the anti-C3 LCDR3 comprises the amino acid sequence of SEQ ID NO: 897.

190. The bispecific antibody of any one of claims 16 to 18, wherein the anti-C3 LCDR1 comprises the amino acid sequence of SEQ ID NO: 835; the anti-C3 LCDR2 comprises the amino acid sequence of SEQ ID NO: 867; and the anti-C3 LCDR3 comprises the amino acid sequence of SEQ ID NO: 898.

191. The bispecific antibody of any one of claims 16 to 18, wherein the anti-C3 LCDR1 comprises the amino acid sequence of SEQ ID NO: 836; the anti-C3 LCDR2 comprises the amino acid sequence of SEQ ID NO: 868; and the anti-C3 LCDR3 comprises the amino acid sequence of SEQ ID NO: 899.

192. The bispecific antibody of any one of claims 16 to 18, wherein the anti-C3 LCDR1 comprises the amino acid sequence of SEQ ID NO: 837; the anti-C3 LCDR2 comprises the amino acid sequence of SEQ ID NO: 869; and the anti-C3 LCDR3 comprises the amino acid sequence of SEQ ID NO: 900.

193. The bispecific antibody of any one of claims 16 to 18, wherein the anti-C3 LCDR1 comprises the amino acid sequence of SEQ ID NO: 838; the anti-C3 LCDR2 comprises the amino acid sequence of SEQ ID NO: 870; and the anti-C3 LCDR3 comprises the amino acid sequence of SEQ ID NO: 901.

194. The bispecific antibody of any one of claims 16 to 18, wherein the anti-C3 LCDR1 comprises the amino acid sequence of SEQ ID NO: 839; the anti-C3 LCDR2 comprises the amino acid sequence of SEQ ID NO: 871; and the anti-C3 LCDR3 comprises the amino acid sequence of SEQ ID NO: 902.

195. The bispecific antibody of any one of claims 16 to 18, wherein the anti-C3 LCDR1 comprises the amino acid sequence of SEQ ID NO: 840; the anti-C3 LCDR2 comprises the amino acid sequence of SEQ ID NO: 872; and the anti-C3 LCDR3 comprises the amino acid sequence of SEQ ID NO: 903.

196. The bispecific antibody of any one of claims 16 to 18, wherein the anti-C3 LCDR1 comprises the amino acid sequence of SEQ ID NO: 841; the anti-C3 LCDR2 comprises the amino acid sequence of SEQ ID NO: 873; and the anti-C3 LCDR3 comprises the amino acid sequence of SEQ ID NO: 904.

197. The bispecific antibody of any one of claims 16 to 18, wherein the anti-C3 LCDR1 comprises the amino acid sequence of SEQ ID NO: 842; the anti-C3 LCDR2 comprises the amino acid sequence of SEQ ID NO: 874; and the anti-C3 LCDR3 comprises the amino acid sequence of SEQ ID NO: 905.

198. The bispecific antibody of any one of claims 16 to 18, wherein the anti-C3 LCDR1 comprises the amino acid sequence of SEQ ID NO: 843; the anti-C3 LCDR2 comprises the amino acid sequence of SEQ ID NO: 875; and the anti-C3 LCDR3 comprises the amino acid sequence of SEQ ID NO: 906.

199. The bispecific antibody of any one of claims 16 to 18, wherein the anti-C3 LCDR1 comprises the amino acid sequence of SEQ ID NO: 844; the anti-C3 LCDR2 comprises the amino acid sequence of SEQ ID NO: 876; and the anti-C3 LCDR3 comprises the amino acid sequence of SEQ ID NO: 907.

200. The bispecific antibody of any one of claims 16 to 18, wherein the anti-C3 LCDR1 comprises the amino acid sequence of SEQ ID NO: 845; the anti-C3 LCDR2 comprises the amino acid sequence of SEQ ID NO: 877; and the anti-C3 LCDR3 comprises the amino acid sequence of SEQ ID NO: 908.

201. The bispecific antibody of any one of claims 16 to 18, wherein the anti-C3 LCDR1 comprises the amino acid sequence of SEQ ID NO: 846; the anti-C3 LCDR2 comprises the amino acid sequence of SEQ ID NO: 878; and the anti-C3 LCDR3 comprises the amino acid sequence of SEQ ID NO: 909.

202. The bispecific antibody of any one of claims 16 to 18, wherein the anti-C3 LCDR1 comprises the amino acid sequence of SEQ ID NO: 847; the anti-C3 LCDR2 comprises the amino acid sequence of SEQ ID NO: 879; and the anti-C3 LCDR3 comprises the amino acid sequence of SEQ ID NO: 910.

203. The bispecific antibody of any one of claims 16 to 18, wherein the anti-C3 LCDR1 comprises the amino acid sequence of SEQ ID NO: 848; the anti-C3 LCDR2 comprises the amino acid sequence of SEQ ID NO: 880; and the anti-C3 LCDR3 comprises the amino acid sequence of SEQ ID NO: 911.

204. The bispecific antibody of any one of claims 16 to 18, wherein the anti-C3 LCDR1 comprises the amino acid sequence of SEQ ID NO: 913; the anti-C3 LCDR2 comprises the amino acid sequence of SEQ ID NO: 916; and the anti-C3 LCDR3 comprises the amino acid sequence of SEQ ID NO: 919.

205. The bispecific antibody of any one of claims 16 to 18, wherein the anti-C3 LCDR1 comprises the amino acid sequence of SEQ ID NO: 914; the anti-C3 LCDR2 comprises the amino acid sequence of SEQ ID NO: 917; and the anti-C3 LCDR3 comprises the amino acid sequence of SEQ ID NO: 920.

206. The bispecific antibody of any one of claims 16 to 18, wherein the anti-C3 LCDR1 comprises the amino acid sequence of SEQ ID NO: 914; the anti-C3 LCDR2 comprises the amino acid sequence of SEQ ID NO: 917; and the anti-C3 LCDR3 comprises the amino acid sequence of SEQ ID NO: 920.

207. The bispecific antibody of any one of claims 16 to 18, wherein the anti-C3 LCDR1 comprises the amino acid sequence of SEQ ID NO: 922; the anti-C3 LCDR2 comprises the amino acid sequence of SEQ ID NO: 931; and the anti-C3 LCDR3 comprises the amino acid sequence of SEQ ID NO: 940.

208. The bispecific antibody of any one of claims 16 to 18, wherein the anti-C3 LCDR1 comprises the amino acid sequence of SEQ ID NO: 923; the anti-C3 LCDR2 comprises the amino acid sequence of SEQ ID NO: 932; and the anti-C3 LCDR3 comprises the amino acid sequence of SEQ ID NO: 941.

209. The bispecific antibody of any one of claims 16 to 18, wherein the anti-C3 LCDR1 comprises the amino acid sequence of SEQ ID NO: 924; the anti-C3 LCDR2 comprises the amino acid sequence of SEQ ID NO: 933; and the anti-C3 LCDR3 comprises the amino acid sequence of SEQ ID NO: 942.

210. The bispecific antibody of any one of claims 16 to 18, wherein the anti-C3 LCDR1 comprises the amino acid sequence of SEQ ID NO: 925; the anti-C3 LCDR2 comprises the amino acid sequence of SEQ ID NO: 934; and the anti-C3 LCDR3 comprises the amino acid sequence of SEQ ID NO: 943.

211. The bispecific antibody of any one of claims 16 to 18, wherein the anti-C3 LCDR1 comprises the amino acid sequence of SEQ ID NO: 926; the anti-C3 LCDR2 comprises the amino acid sequence of SEQ ID NO: 935; and the anti-C3 LCDR3 comprises the amino acid sequence of SEQ ID NO: 944.

212. The bispecific antibody of any one of claims 16 to 18, wherein the anti-C3 LCDR1 comprises the amino acid sequence of SEQ ID NO: 927; the anti-C3 LCDR2 comprises the amino acid sequence of SEQ ID NO: 936; and the anti-C3 LCDR3 comprises the amino acid sequence of SEQ ID NO: 945.

213. The bispecific antibody of any one of claims 16 to 18, wherein the anti-C3 LCDR1 comprises the amino acid sequence of SEQ ID NO: 928; the anti-C3 LCDR2 comprises the amino acid sequence of SEQ ID NO: 937; and the anti-C3 LCDR3 comprises the amino acid sequence of SEQ ID NO: 946.

214. The bispecific antibody of any one of claims 16 to 18, wherein the anti-C3 LCDR1 comprises the amino acid sequence of SEQ ID NO: 929; the anti-C3 LCDR2 comprises the amino acid sequence of SEQ ID NO: 938; and the anti-C3 LCDR3 comprises the amino acid sequence of SEQ ID NO: 947.

215. The bispecific antibody of any one of claims 16 to 18, wherein the anti-C3 LCDR1 comprises the amino acid sequence of SEQ ID NO: 949; the anti-C3 LCDR2 comprises the amino acid sequence of SEQ ID NO: 984; and the anti-C3 LCDR3 comprises the amino acid sequence of SEQ ID NO: 1019.

216. The bispecific antibody of any one of claims 16 to 18, wherein the anti-C3 LCDR1 comprises the amino acid sequence of SEQ ID NO: 950; the anti-C3 LCDR2 comprises the amino acid sequence of SEQ ID NO: 985; and the anti-C3 LCDR3 comprises the amino acid sequence of SEQ ID NO: 1020.

217. The bispecific antibody of any one of claims 16 to 18, wherein the anti-C3 LCDR1 comprises the amino acid sequence of SEQ ID NO: 951; the anti-C3 LCDR2 comprises the amino acid sequence of SEQ ID NO: 986; and the anti-C3 LCDR3 comprises the amino acid sequence of SEQ ID NO: 1021.

218. The bispecific antibody of any one of claims 16 to 18, wherein the anti-C3 LCDR1 comprises the amino acid sequence of SEQ ID NO: 952; the anti-C3 LCDR2 comprises the amino acid sequence of SEQ ID NO: 987; and the anti-C3 LCDR3 comprises the amino acid sequence of SEQ ID NO: 1022.

219. The bispecific antibody of any one of claims 16 to 18, wherein the anti-C3 LCDR1 comprises the amino acid sequence of SEQ ID NO: 953; the anti-C3 LCDR2 comprises the amino acid sequence of SEQ ID NO: 988; and the anti-C3 LCDR3 comprises the amino acid sequence of SEQ ID NO: 1023.

220. The bispecific antibody of any one of claims 16 to 18, wherein the anti-C3 LCDR1 comprises the amino acid sequence of SEQ ID NO: 954; the anti-C3 LCDR2 comprises the amino acid sequence of SEQ ID NO: 989; and the anti-C3 LCDR3 comprises the amino acid sequence of SEQ ID NO: 1024.

221. The bispecific antibody of any one of claims 16 to 18, wherein the anti-C3 LCDR1 comprises the amino acid sequence of SEQ ID NO: 955; the anti-C3 LCDR2 comprises the amino acid sequence of SEQ ID NO: 990; and the anti-C3 LCDR3 comprises the amino acid sequence of SEQ ID NO: 1025.

222. The bispecific antibody of any one of claims 16 to 18, wherein the anti-C3 LCDR1 comprises the amino acid sequence of SEQ ID NO: 956; the anti-C3 LCDR2 comprises the amino acid sequence of SEQ ID NO: 991; and the anti-C3 LCDR3 comprises the amino acid sequence of SEQ ID NO: 1026.

223. The bispecific antibody of any one of claims 16 to 18, wherein the anti-C3 LCDR1 comprises the amino acid sequence of SEQ ID NO: 957; the anti-C3 LCDR2 comprises the amino acid sequence of SEQ ID NO: 992; and the anti-C3 LCDR3 comprises the amino acid sequence of SEQ ID NO: 1027.

224. The bispecific antibody of any one of claims 16 to 18, wherein the anti-C3 LCDR1 comprises the amino acid sequence of SEQ ID NO: 958; the anti-C3 LCDR2 comprises the amino acid sequence of SEQ ID NO: 993; and the anti-C3 LCDR3 comprises the amino acid sequence of SEQ ID NO: 1028.

225. The bispecific antibody of any one of claims 16 to 18, wherein the anti-C3 LCDR1 comprises the amino acid sequence of SEQ ID NO: 959; the anti-C3 LCDR2 comprises the amino acid sequence of SEQ ID NO: 994; and the anti-C3 LCDR3 comprises the amino acid sequence of SEQ ID NO: 1029.

226. The bispecific antibody of any one of claims 16 to 18, wherein the anti-C3 LCDR1 comprises the amino acid sequence of SEQ ID NO: 960; the anti-C3 LCDR2 comprises the amino acid sequence of SEQ ID NO: 995; and the anti-C3 LCDR3 comprises the amino acid sequence of SEQ ID NO: 1030.

227. The bispecific antibody of any one of claims 16 to 18, wherein the anti-C3 LCDR1 comprises the amino acid sequence of SEQ ID NO: 961; the anti-C3 LCDR2 comprises the amino acid sequence of SEQ ID NO: 996; and the anti-C3 LCDR3 comprises the amino acid sequence of SEQ ID NO: 1031.

228. The bispecific antibody of any one of claims 16 to 18, wherein the anti-C3 LCDR1 comprises the amino acid sequence of SEQ ID NO: 962; the anti-C3 LCDR2 comprises the amino acid sequence of SEQ ID NO: 997; and the anti-C3 LCDR3 comprises the amino acid sequence of SEQ ID NO: 1032.

229. The bispecific antibody of any one of claims 16 to 18, wherein the anti-C3 LCDR1 comprises the amino acid sequence of SEQ ID NO: 963; the anti-C3 LCDR2 comprises the amino acid sequence of SEQ ID NO: 998; and the anti-C3 LCDR3 comprises the amino acid sequence of SEQ ID NO: 1033.

230. The bispecific antibody of any one of claims 16 to 18, wherein the anti-C3 LCDR1 comprises the amino acid sequence of SEQ ID NO: 964; the anti-C3 LCDR2 comprises the amino acid sequence of SEQ ID NO: 999; and the anti-C3 LCDR3 comprises the amino acid sequence of SEQ ID NO: 1034.

231. The bispecific antibody of any one of claims 16 to 18, wherein the anti-C3 LCDR1 comprises the amino acid sequence of SEQ ID NO: 965; the anti-C3 LCDR2 comprises the amino acid sequence of SEQ ID NO: 1000; and the anti-C3 LCDR3 comprises the amino acid sequence of SEQ ID NO: 1035.

232. The bispecific antibody of any one of claims 16 to 18, wherein the anti-C3 LCDR1 comprises the amino acid sequence of SEQ ID NO: 966; the anti-C3 LCDR2 comprises the amino acid sequence of SEQ ID NO: 1001; and the anti-C3 LCDR3 comprises the amino acid sequence of SEQ ID NO: 1036.

233. The bispecific antibody of any one of claims 16 to 18, wherein the anti-C3 LCDR1 comprises the amino acid sequence of SEQ ID NO: 967; the anti-C3 LCDR2 comprises the amino acid sequence of SEQ ID NO: 1002; and the anti-C3 LCDR3 comprises the amino acid sequence of SEQ ID NO: 1037.

234. The bispecific antibody of any one of claims 16 to 18, wherein the anti-C3 LCDR1 comprises the amino acid sequence of SEQ ID NO: 968; the anti-C3 LCDR2 comprises the amino acid sequence of SEQ ID NO: 1003; and the anti-C3 LCDR3 comprises the amino acid sequence of SEQ ID NO: 1038.

235. The bispecific antibody of any one of claims 16 to 18, wherein the anti-C3 LCDR1 comprises the amino acid sequence of SEQ ID NO: 969; the anti-C3 LCDR2 comprises the amino acid sequence of SEQ ID NO: 1004; and the anti-C3 LCDR3 comprises the amino acid sequence of SEQ ID NO: 1039.

236. The bispecific antibody of any one of claims 16 to 18, wherein the anti-C3 LCDR1 comprises the amino acid sequence of SEQ ID NO: 970; the anti-C3 LCDR2 comprises the amino acid sequence of SEQ ID NO: 1005; and the anti-C3 LCDR3 comprises the amino acid sequence of SEQ ID NO: 1040.

237. The bispecific antibody of any one of claims 16 to 18, wherein the anti-C3 LCDR1 comprises the amino acid sequence of SEQ ID NO: 971; the anti-C3 LCDR2 comprises the amino acid sequence of SEQ ID NO: 1006; and the anti-C3 LCDR3 comprises the amino acid sequence of SEQ ID NO: 1041.

238. The bispecific antibody of any one of claims 16 to 18, wherein the anti-C3 LCDR1 comprises the amino acid sequence of SEQ ID NO: 972; the anti-C3 LCDR2 comprises the amino acid sequence of SEQ ID NO: 1007; and the anti-C3 LCDR3 comprises the amino acid sequence of SEQ ID NO: 1042.

239. The bispecific antibody of any one of claims 16 to 18, wherein the anti-C3 LCDR1 comprises the amino acid sequence of SEQ ID NO: 973; the anti-C3 LCDR2 comprises the amino acid sequence of SEQ ID NO: 1008; and the anti-C3 LCDR3 comprises the amino acid sequence of SEQ ID NO: 1043.

240. The bispecific antibody of any one of claims 16 to 18, wherein the anti-C3 LCDR1 comprises the amino acid sequence of SEQ ID NO: 974; the anti-C3 LCDR2 comprises the amino acid sequence of SEQ ID NO: 1009; and the anti-C3 LCDR3 comprises the amino acid sequence of SEQ ID NO: 1044.

241. The bispecific antibody of any one of claims 16 to 18, wherein the anti-C3 LCDR1 comprises the amino acid sequence of SEQ ID NO: 975; the anti-C3 LCDR2 comprises the amino acid sequence of SEQ ID NO: 1010; and the anti-C3 LCDR3 comprises the amino acid sequence of SEQ ID NO: 1045.

242. The bispecific antibody of any one of claims 16 to 18, wherein the anti-C3 LCDR1 comprises the amino acid sequence of SEQ ID NO: 976; the anti-C3 LCDR2 comprises the amino acid sequence of SEQ ID NO: 1011; and the anti-C3 LCDR3 comprises the amino acid sequence of SEQ ID NO: 1046.

243. The bispecific antibody of any one of claims 16 to 18, wherein the anti-C3 LCDR1 comprises the amino acid sequence of SEQ ID NO: 977; the anti-C3 LCDR2 comprises the amino acid sequence of SEQ ID NO: 1012; and the anti-C3 LCDR3 comprises the amino acid sequence of SEQ ID NO: 1047.

244. The bispecific antibody of any one of claims 16 to 18, wherein the anti-C3 LCDR1 comprises the amino acid sequence of SEQ ID NO: 978; the anti-C3 LCDR2 comprises the amino acid sequence of SEQ ID NO: 1013; and the anti-C3 LCDR3 comprises the amino acid sequence of SEQ ID NO: 1048.

245. The bispecific antibody of any one of claims 16 to 18, wherein the anti-C3 LCDR1 comprises the amino acid sequence of SEQ ID NO: 979; the anti-C3 LCDR2 comprises the amino acid sequence of SEQ ID NO: 1014; and the anti-C3 LCDR3 comprises the amino acid sequence of SEQ ID NO: 1049.

246. The bispecific antibody of any one of claims 16 to 18, wherein the anti-C3 LCDR1 comprises the amino acid sequence of SEQ ID NO: 980; the anti-C3 LCDR2 comprises the amino acid sequence of SEQ ID NO: 1015; and the anti-C3 LCDR3 comprises the amino acid sequence of SEQ ID NO: 1050.

247. The bispecific antibody of any one of claims 16 to 18, wherein the anti-C3 LCDR1 comprises the amino acid sequence of SEQ ID NO: 981; the anti-C3 LCDR2 comprises the amino acid sequence of SEQ ID NO: 1016; and the anti-C3 LCDR3 comprises the amino acid sequence of SEQ ID NO: 1051.

248. The bispecific antibody of any one of claims 16 to 18, wherein the anti-C3 LCDR1 comprises the amino acid sequence of SEQ ID NO: 982; the anti-C3 LCDR2 comprises the amino acid sequence of SEQ ID NO: 1017; and the anti-C3 LCDR3 comprises the amino acid sequence of SEQ ID NO: 1052.

249. The bispecific antibody of any one of claims 16 to 18, wherein the anti-CFHR4 VH comprises an amino acid sequence having at least 90% sequence identity to at least one of SEQ ID NOs: 260-265, 284-293, 324-334, and 368-380; and wherein the anti-CFHR4 VL comprises an amino acid sequence having at least 90% sequence identity to at least one of SEQ ID NOs: 272-277, 304-313, 346-356, and 394-406.

250. The bispecific antibody of any one of claims 16 to 18, wherein the anti-C3 VH comprises an amino acid sequence having at least 90% sequence identity to at least one of SEQ ID NOs: 1127-1200; and wherein the anti-C3 VL comprises an amino acid sequence having at least 90% sequence identity to at least one of SEQ ID NOs: 1275-1348.

251. The bispecific antibody of any one of claims 1 to 250, wherein the antibody binds CFHR4 and/or C3 and reduces complement activation.

252. The bispecific antibody of claim 251, wherein the antibody binds CFHR4b and/or C3b and reduces complement activation.

253. The bispecific antibody of any one of claims 1 to 252, wherein the antibody binds human CFHR4 and/or C3 with a Ko of about 100 nM or lower.

254. The bispecific antibody of any one of claims 1 to 253, wherein the antibody comprises a half-life extension moiety.

255. The bispecific antibody of claim 254, wherein the half-life extension moiety comprises a polypeptide that is at least 20 amino acids in length and comprises any combination of G, A, S T, E, and P residues.

256. The antibody of claim 255, wherein the half-life extension polypeptide is attached to the C-terminus or N-terminus of the antibody.

257. A pharmaceutical composition comprising any of the antibodies of claims 1 to 256.

258. The composition of claim 257, wherein the composition is suitable for ocular administration.

259. The composition of claim 258, wherein the ocular administration comprises injection into vitreous fluid.

260. The composition of claim 258, wherein the ocular administration comprises delivering the antibody using a conjunctival insert, a contact lens, a gel, a nanoparticle, a mucoadhesive polymer, an ointment, a solution, a suspension, eye drops, and/or an implant.

261. A method of treating age-related macular degeneration (AMD) comprising administering a pharmaceutical composition comprising an effective amount of the antibody of any one of claims 1 to 256 to a subject in need thereof.

262. The method of claim 261, wherein the pharmaceutical composition is administered ocularly and treats at least one AMD symptom.

263. The method of claim 261, wherein AMD comprises wet AMD.

264. The method of claim 261, wherein AMD comprises dry AMD.

265. The method of any one of claims 261 to 264, wherein the at least one AMD symptom comprises visual distortion, reduced central vision, blurred vision, and/or difficulty adapting to low light.

266. The method of any one of claims 261 to 265, wherein administering the pharmaceutical composition reduces complement activation in the subject's eye.

267. The method of any one of claims 261 to 266, wherein the pharmaceutical composition is administered at a dose ranging from about 0.0001 mg/dose to about 100 mg/dose.

268. The method of any one of claims 261 to 266, wherein the pharmaceutical composition is administered at a dose ranging from about 0.0001 mg/ml to about 100 mg/ml.
